(19) United States
(12) Patent Application Publication
(10) Pub. No.: US 2003/0077701 A1
(43) Pub. Date: Apr. 24, 2003

(54) DNA encoding a human melanin concentrating hormone receptor (MCH1) and uses thereof

(76) Inventors: Carlos Forray, Paramus, NJ (US); John A. Salon, Santa Paula, CA (US); Thomas M. Laz, Parlin, NJ (US); Raisa Nagorny, Fairlawn, NY (US); Amy E. Wilson, Woodstock, NY (US)

Correspondence Address: John P. White, Cooper & Dunham LLP, 1185 Avenue of the Americas, New York, NY, 10036, US

(21) Appl. No.: 10/029,314

(22) Filed: Dec. 20, 2001

Related U.S. Application Data
(63) Continuation of application No. 09/899,732, filed on Jul. 5, 2001.
Application No. 09/899,732 is a continuation-in-part of application No. 09/610,635, filed on Jul. 5, 2000, now abandoned.
Application No. 09/610,635 is a continuation-in-part of application No. PCT/US99/31169, filed on Dec. 30, 1999.
Application No. PCT/US99/31169 is a continuation-in-part of application No. 09/224,426, filed on Dec. 31, 1998, now Pat. No. 6,221,613.

(51) Int. Cl. C07K 14/74; C07H 21/04; C12P 21/02; C12N 5/06

(52) U.S. Cl. 435/69.1; 435/320.1; 435/325; 530/350; 536/23.5

(57) ABSTRACT

This invention provides an isolated nucleic acid encoding a human MCH1 receptor, a purified human MCH1 receptor, vectors comprising isolated nucleic acid encoding a human MCH1 receptor, cells comprising such vectors, antibodies directed to a human MCH1 receptor, nucleic acid probes useful for detecting nucleic acid encoding human MCH1 receptors, antisense oligonucleotides complementary to unique sequences of nucleic acid encoding human MCH1 receptors, transgenic, nonhuman animals which express DNA encoding a normal or mutant human MCH1 receptor, methods of isolating a human MCH1 receptor, methods of treating an abnormality that is linked to the activity of a human MCH1 receptor, as well as methods of determining binding of compounds to mammalian MCH1 receptors. This invention provides a method of modifying the feeding behavior of a subject which comprises administering to the subject an amount of an MCH1 antagonist effective to decrease the body mass of the subject and/or decrease the consumption of food by the subject. This invention further provides a method of treating a subject suffering from depression and/or anxiety which comprises administering to the subject an amount of an MCH1 antagonist effective to treat the subject's depression and/or anxiety.

This application is a continuation-in-part of U.S. Ser. No. 09/610,635, filed Jul. 5, 2000, which is a continuation-in-part of PCT International Application No. PCT/US99/31169, filed Dec. 30, 1999, which is a continuation-in-part of U.S. Ser. No. 09/224,426, filed Dec. 31, 1998, the contents of which are hereby incorporated by reference into the subject application.

### BACKGROUND OF THE INVENTION

Throughout this application, various publications are referenced in parentheses by author and year. Full citations for these references may be found at the end of the specification immediately preceding the sequence listings and the claims. The disclosure of these publications in their entireties are hereby incorporated by reference into this application to describe more fully the state of the art to which this invention pertains.

Neuroregulators comprise a diverse group of natural products that subserve or modulate communication in the nervous system. They include, but are not limited to, neuropeptides, amino acids, biogenic amines, lipids and lipid metabolites, and other metabolic byproducts. Many of these neuroregulator substances interact with specific cell surface receptors which transduce signals from the outside to the inside of the cell. G-protein coupled receptors (GPCRs) represent a major class of cell surface receptors with which many neurotransmitters interact to mediate their effects. GPCRs are predicted to have seven membrane-spanning domains and are coupled to their effectors via G-proteins linking receptor activation with intracellular biochemical sequelae such as stimulation of adenylyl cyclase. Melanin-concentrating hormone (MCH) is a cyclic peptide originally isolated from salmonid (teleost fish) pituitaries (Kawauchi et al., 1983). In fish the 17 amino acid peptide causes aggregation of melanin within the melanophores and inhibits the release of ACTH, acting as a functional antagonist of α-MSH. Mammalian MCH (19 amino acids) is highly conserved between rat, mouse, and human, exhibiting 100% amino acid identity, but its physiological roles are less clear. MCH has been reported to participate in a variety of processes including feeding, water balance, energy metabolism, general arousal/attention state, memory and cognitive functions, and psychiatric disorders (for reviews, see Baker, 1991; Baker, 1994; Nahon, 1994; Knigge et al., 1996). Its role in feeding or body weight regulation is supported by a recent Nature publication (Qu et al., 1996) demonstrating that MCH is overexpressed in the hypothalamus of ob/ob mice compared with ob/+ mice, and that fasting further increased MCH mRNA in both obese and normal mice during fasting. MCH also stimulated feeding in normal rats when injected into the lateral ventricles (Rossi et al., 1997). MCH also has been reported to functionally antagonize the behavioral effects of α-MSH (Miller et al., 1993; Gonzalez et al, 1996; Sanchez et al., 1997); in addition, stress has been shown to increase POMC mRNA levels while decreasing the MCH precursor preproMCH (ppMCH) mRNA levels (Presse et al., 1992). Thus MCH may serve as an integrative neuropeptide involved in the reaction to stress, as well as in the regulation of feeding and sexual activity (Baker, 1991; Knigge et al., 1996). The gene encoding the MCH precursor (ppMCH) has been cloned and encodes two additional peptides, neuropeptide EI (13 AA) and neuropeptide GE (19AA) (Nahon et al., 1989), which may also have biological activity. MCH peptide is synthesized primarily in hypothalamic neurons (the zona incerta and lateral hypothalamus) which project diffusely to many brain areas and to the pituitary (Bittencourt et al., 1992); NEI has also been identified in medium from explanted hypothalamic neurons (Parkes and Vale, 1993). Localization studies of the mRNA indicate that MCH is also present in the periphery (testes and GI tract; Hervieu and Nahon, 1995) but the highest concentrations are in the hypothalamus. There is also evidence for differential tissue-dependent processing of proMCH in mammals. A shorter MCH gene transcript that may result from alternate splicing was found in several brain areas and peripheral tissues, and a different peptide form was also found in the periphery (Viale et al., 1997). In humans, the gene encoding authentic MCH has been localized to chromosome 12, but two copies of a variant (truncated) gene are present on chromosome 5 (Breton et al., 1993); the functional significance, if any, of the variant is not yet known. Finally, the rat MCH gene may encode an additional putative peptide in a different reading frame (Toumaniantz et al., 1996).

Although the biological effects of MCH are believed to be mediated by specific receptors, binding sites for MCH have not been well described. A tritiated ligand ([³H]-MCH) was reported to exhibit specific binding to brain membranes but was unusable for saturation analyses, so neither affinity nor Bₘₐₓ were determined (Drozdz and Eberle, 1995). Radioiodination of the tyrosine at position thirteen resulted in a ligand with dramatically reduced biological activity (see Drozdz and Eberle, 1995). In contrast, the radioiodination of the MCH analogue [Phe¹³,Tyr¹⁹]-MCH was successful (Drozdz et al., 1995); the ligand retained biological activity and exhibited specific binding to a variety of cell lines including mouse melanoma (Bl6-Fl, G4F, and G4F-7), PC12, and COS cells. In G4F-7 cells, the K_{D}=0.118nM and the Bₘₐₓ˜1100 sites/cell. Importantly, the binding was not inhibited by α-MSH but was weakly inhibited by rat ANF (Ki=116 nM vs. 12 nM for native MCH) (Drozdz et al., 1995). More recently specific MCH binding was reported in transformed keratinocytes (Burgaud et al., 1997) and melanoma cells (Drozdz et al., 1998), where photo-crosslinking studies suggest that the receptor is a membrane protein with an apparent molecular weight of 45-50 kDaltons, compatible with the molecular weight range of the GPCR superfamily of receptors. No radioautoradiographic studies of MCH receptor localization using this ligand have been reported as yet.

Signal transduction mechanisms for MCH receptors remain obscure. No direct evidence supporting G-protein coupling exists in mammals, but two lines of weak evidence exist in teleost fish for G_{αq}- and/or G_{αi}-type coupling: 1) indirect evidence exists for MCH acting via phospholipase C in teleost fish melanophores (phospholipase C inhibitors and protein kinase C inhibitors shift the MCH dose-response curve to the right, and TPA mimics MCH at low doses (Abrao et al., 1991)); and 2) MCH-elicited pigment aggregation in fish melanophores is associated with a reduction in basal cAMP levels, similar to that observed with norepinephrine (Svensson et al., 1991; Morishita et al., 1993). Arguing against G-protein coupling is the general structural homology of MCH with ANF, whose receptors are not in the GPCR superfamily. Recently the actions of MCH were reported to be mediated via activation of a phosphatidylinositol-3-kinase pathway which is typical of tyrosine kinase and cytokine receptors (Qu et al., 1998); however, since multiple signaling pathways (receptor cross talk) may produce this mediator no conclusions can be reached regarding MCH signal transduction pathways in mammalian systems.

The localization and biological activities of MCH peptide suggest that the modulation of MCH receptor activity may be useful in a number of therapeutic applications. The role of MCH in feeding is the best characterized of its potential clinical uses. MCH is expressed in the lateral hypothalamus, a brain area implicated in the regulation of thirst and hunger (Grillon et al., 1997); recently orexins A and B, which are potent orexigenic agents, have been shown to have very similar localization to MCH in the lateral hypothalamus (Sakurai et al., 1998). MCH mRNA levels in this brain region are increased in rats after 24 hours of food-deprivation (Herve and Fellman, 1997); after insulin injection, a significant increase in the abundance and staining intensity of MCH immunoreactive perikarya and fibres was observed concurrent with a significant increase in the level of MCH mRNA (Bahjaoui-Bouhaddi et al., 1994). Consistent with the ability of MCH to stimulate feeding in rats (Rossi et al., 1997) is the observation that MCH mRNA levels are upregulated in the hypothalami of obese ob/ob mice (Qu et al., 1996), and decreased in the hypothalami of rats treated with leptin, whose food intake and body weight gains are also decreased (Sahu, 1998). MCH appears to act as a functional antagonist of the melanocortin system in its effects on food intake and on hormone secretion within the HPA (hypothalamopituitary /adrenal axis) (Ludwig et al., 1998). Further evidence of the involvement of MCH in the regulation of feeding behavior came from studies in mice in which the gene encoding the MCH peptide has been deleted (Shimada et al., 1998). In these mice, the genetic deficiency of MCH led to a phenotype characterized by reduced body weight, low body fat content, and increased metabolic rate. More recently, it has been shown that the overexpression of the gene encoding MCH in different strains of mice can lead to obese phenotypes with and without secondary impairment of glucose homeostasis and insulin resistance (Tritos et al., 2000).

Together these data suggest a role for endogenous MCH in the regulation of energy balance and response to stress, and provide a rationale for the development of specific compounds acting at MCH receptors for use in the treatment of obesity and stress-related disorders.

In all species studied to date, a major portion of the neurons of the MCH cell group occupies a rather constant location in those areas of the lateral hypothalamus and subthalamus where they lie and may be a part of some of the so-called “extrapyramidal” motor circuits. These involve substantial striato- and pallidofugal pathways involving the thalamus and cerebral cortex, hypothalamic areas, and reciprocal connections to subthalamic nucleus, substantia nigra, and mid-brain centers (Bittencourt et al., 1992). In their location, the MCH cell group may offer a bridge or mechanism for expressing hypothalamic visceral activity with appropriate and coordinated motor activity. Clinically it may be of some value to consider the involvement of this MCH system in movement disorders, such as Parkinson's disease and Huntingdon's Chorea in which extrapyramidal circuits are known to be involved.

Human genetic linkage studies have located authentic hMCH loci on chromosome 12 (12q23-24) and the variant hMCH loci on chromosome 5 (5q12-13) (Pedeutour et al., 1994). Locus 12q23-24 coincides with a locus to which autosomal dominant cerebellar ataxia type II (SCA2) has been mapped (Auburger et al., 1992; Twells et al., 1992). This disease comprises neurodegenerative disorders, including an olivopontocerebellar atrophy. Furthermore, the gene for Darier's disease, has been mapped to locus 12q23-24 (Craddock et al., 1993). Dariers' disease is characterized by abnormalities I keratinocyte adhesion and mental illnesses in some families. In view of the functional and neuroanatomical patterns of the MCH neural system in the rat and human brains, the MCH gene may represent a good candidate for SCA2 or Darier's disease. Interestingly, diseases with high social impact have been mapped to this locus. Indeed, the gene responsible for chronic or acute forms of spinal muscular atrophies has been assigned to chromosome 5q12-13 using genetic linkage analysis (Melki et al., 1990; Westbrook et al., 1992). Furthermore, independent lines of evidence support the assignment of a major schizophrenia locus to chromosome 5q11.2-13.3 (Sherrington et al., 1988; Bassett et al., 1988; Gilliam et al., 1989). The above studies suggest that MCH may play a role in neurodegenerative diseases and disorders of emotion.

Additional therapeutic applications for MCH-related compounds are suggested by the observed effects of MCH in other biological systems. For example, MCH may regulate reproductive functions in male and female rats. MCH transcripts and MCH peptide were found within germ cells in testes of adult rats, suggesting that MCH may participate in stem cell renewal and/or differentiation of early spermatocytes (Hervieu et al., 1996). MCH injected directly into the medial preoptic area (MPOA) or ventromedial nucleus (VMN) stimulated sexual activity in female rats (Gonzalez et al., 1996). In ovariectomized rats primed with estradiol, MCH stimulated luteinizing hormone (LH) release while anti-MCH antiserum inhibited LH release (Gonzalez et al., 1997). The zona incerta, which contains a large population of MCH cell bodies, has previously been identified as a regulatory site for the pre-ovulatory LH surge (MacKenzie et al., 1984). MCH has been reported to influence release of pituitary hormones including ACTH and oxytocin. MCH analogues may also be useful in treating epilepsy. In the PTZ seizure model, injection of MCH prior to seizure induction prevented seizure activity in both rats and guinea pigs, suggesting that MCH-containing neurons may participate in the neural circuitry underlying PTZ-induced seizure (Knigge and Wagner, 1997). MCH has also been observed to affect behavioral correlates of cognitive functions. MCH treatment hastened extinction of the passive avoidance response in rats (McBride et al., 1994), raising the possibility that MCH receptor antagonists may be beneficial for memory storage and/or retention. A possible role for MCH in the modulation or perception of pain is supported by the dense innervation of the periaqueductal grey (PAG) by MCH-positive fibers. Finally, MCH may participate in the regulation of fluid intake. ICV infusion of MCH in conscious sheep produced diuretic, natriuretic, and kaliuretic changes in response to increased plasma volume (Parkes, 1996). Together with anatomical data reporting the presence of MCH in fluid regulatory areas of the brain, the results indicate that MCH may be an important peptide involved in the central control of fluid homeostasis in mammals.

In light of the localization of MCH1 throughout limbic regions of the rat CNS as described hereinafter, a series of in vivo behavioral experiments were carried out to evaluate the antidepressant and anxiolytic properties of a selective MCH1 receptor antagonist. The rat Forced Swim Test and the rat Social Interaction Test were employed to evaluate the use of selective MCH1 receptor antagonists to treat depression and anxiety. These models are considered by experts in the field to reflect the potential of agents to treat depression and anxiety.

Rat Forced Swim Test (FST)

The rat Forced Swim Test (FST) is a behavioral test that is used to screen compounds for antidepressant efficacy (Porsolt et al., 1977, 1978; Porsolt, 1981). This test is widely used as it is reliable across laboratories, relatively easy to perform and is sensitive to the effects of some of the major classes of antidepressants drugs, including TCAs and MAOIs, and various atypical antidepressants. Furthermore, this test is relatively selective for antidepressant drugs, as few psychoactive drugs produce similar behavioral actions in the FST.

In the rat FST, animals are placed in a cylinder of water, from which there is no escape, for an extended period of time. Typically, animals will display a range of behaviors such as immobility, climbing, swimming, and diving, with immobility being predominant after several minutes of immersion in the water. Consequently, many past studies have only measured or scored immobility after the administration of the test agent. Unfortunately, this method does not score any other active behaviors that may be produced by potential antidepressants. Thus, if a particular class of antidepressant were to have very little effect on immobility, yet produce characteristic behaviors during the FST, these behaviors would not be scored and the conclusion would be that the compound in question does not possess antidepressant action.

Recently, however, a sampling technique was developed to score active behaviors in the FST, such as swimming, climbing and diving, in addition to immobility (Detke, et al., 1995; Lucki, 1997; Page, et al., 1999; Reneric and Lucki, 1998). This modified sampling technique has indicated that SSRIs, such as fluoxetine, paroxetine and sertraline, significantly decrease immobility and increase swimming time (Detke, et al., 1995; Page, et al., 1999). In contrast, selective reuptake inhibitors of norepinephrine (NE) increase climbing behavior but do not alter swimming time (Detke, et al., 1995; Page, et al., 1999).

Rat Social Interaction Test (SIT)

There are a number of paradigms that have been used to determine whether a compound possesses anxiolytic action. A number of these tests involve food or water deprivation, punishment or measurement of consummatory behavior (see File, et al., 1980, File, 1985, Rodgers, et al., 1997 and Treit, 1985, for review). In addition, in these models, prior conditioning reduces the uncertainty or anxiety. In general, these tests lack ethological validity.

One model that is based upon an unconditioned response that does not involve punishment or deprivation is the Social Interaction Test (SIT) (File and Hyde, 1978, 1979). In this model, rats previously housed singly are placed in a familiar, dimly lit, test arena with weight-matched, novel partners. The principal anxiogenic stimulus under these conditions is the partner novelty, which involves an unconditioned response to a potential threat. After pharmacological treatments, the following behaviors are scored as active social interaction: grooming, sniffing, biting, boxing, wrestling, following, crawling over and crawling under. A wide range of psychoactive drugs have been examined in this paradigm and it has been shown that the social interaction test can distinguish anxiolytics from antidepressants, antipsychotics, analeptics and sedative agents (File, 1985; Guy and Gardner, 1985). This test can detect anxiolytic agents such as the benzodiazepines (File and Hyde, 1978; File and Hyde, 1979; File, 1980), in addition to non-benzodiazepines, including paroxetine and other SSRIs (Lightowler, et al., 1994). Finally, the social interaction test can detect anxiogenic agents, including the inverse benzodiazepine receptor agonists (File, et al., 1982, File and Pellow, 1983; File and Pellow, 1984, File, 1985).

From the binding and functional activity information described hereinafter, it has been unexpectedly discovered that compounds which are MCH1 receptor antagonists are effective in animal models of obesity, depression and anxiety, which are predictive of efficacy in humans. Thus, we demonstrate that MCH1 receptor antagonists provide a novel method to treat obesity. Additionally, we demonstrate that MCH1 receptor antagonists provide a novel method to treat depression and/or anxiety.

### SUMMARY OF THE INVENTION

This invention provides an isolated nucleic acid encoding a human MCH1 receptor or a mutant of such human MCH1 receptor which is activated by MCH or an analog or homolog thereof.

This invention provides a nucleic acid encoding a human MCH1 receptor, wherein the nucleic acid (a) hybridizes to a nucleic acid having the defined sequence shown in FIG. 1 (SEQ ID NO: 1) under low stringency conditions or a sequence complementary thereto and (b) is further characterized by its ability to cause a change in the pH of a culture of CHO cells when an MCH1 ligand is added to the culture and the CHO cells contain the nucleic acid which hybridized to the nucleic acid having the defined sequence or its complement.

This invention provides a purified human MCH1 receptor protein.

This invention provides a vector comprising a nucleic acid encoding a human MCH1 receptor, particularly a vector adapted for expression of the human MCH1 receptor in mammalian or non-mammalian cells. One such vector is a plasmid designated pEXJ.HR-TL231 (ATCC Accession No. 203197) which comprises a nucleotide sequence encoding a human MCH1 receptor.

This invention also provides a cell comprising a vector which comprises a nucleic acid encoding a human MCH1 receptor as well as a membrane preparation isolated from such cells.

This invention further provides a nucleic acid probe comprising at least 15 nucleotides which specifically hybridizes with a nucleic acid encoding a mammalian MCH1 receptor, wherein the probe has a unique sequence corresponding to a sequence present within the nucleic acid which encodes the human MCH1 receptor or its complement, both of which are present in plasmid pEXJ.HR-TL231 (ATCC Accession No. 203197).

This invention further provides a nucleic acid probe comprising at least 15 nucleotides which specifically hybridizes with a nucleic acid encoding a mammalian MCH1 receptor, wherein the probe has a unique sequence corresponding to a sequence present within (a) the nucleic acid sequence shown in FIG. 1 (SEQ ID NO: 1) or (b) the reverse complement thereof.

This invention also provides an antisense oligonucleotide having a sequence capable of specifically hybridizing an RNA encoding a human MCH1 receptor, so as to prevent translation of the RNA and an antisense oligonucleotide having a sequence capable of specifically hybridizing to the genomic DNA encoding a human MCH1 receptor.

This invention further provides an antibody capable of binding to a human MCH1 receptor as well as an agent capable of competitively inhibiting the binding of the antibody to a human MCH1 receptor.

This invention provides a pharmaceutical composition comprising (a) an amount of the oligonucleotide described above capable of passing through a cell membrane and effective to reduce expression of a human MCH1 receptor and (b) a pharmaceutically acceptable carrier capable of passing through the cell membrane.

Moreover, this invention provides a transgenic, nonhuman mammal expressing DNA encoding a human MCH1 receptor. This invention also provides a transgenic, nonhuman mammal comprising a homologous recombination knockout of the native human MCH1 receptor. This invention further provides a transgenic, nonhuman mammal whose genome comprises antisense DNA complementary to the DNA encoding a human MCH1 receptor so placed within the genome as to be transcribed into antisense mRNA which is complementary to mRNA encoding the human MCH1 receptor and which hybridizes to mRNA encoding the human MCH1 receptor, thereby reducing its translation.

In one embodiment this invention provides a process for identifying a chemical compound which specifically binds to a mammalian MCH1 receptor which comprises contacting cells containing DNA encoding and expressing on their cell surface a mammalian MCH1 receptor, wherein such cells do not normally express the mammalian MCH1 receptor, with the compound under conditions suitable for binding, and detecting specific binding of the chemical compound to the mammalian MCH1 receptor.

This invention provides a process for identifying a chemical compound which specifically binds to a mammalian MCH1 receptor which comprises contacting a membrane preparation from cells transfected with DNA encoding and expressing on their cell surface the mammalian MCH1 receptor, wherein such cells do not normally express the mammalian MCH1 receptor, with the compound under conditions suitable for binding, and detecting specific binding of the chemical compound to the mammalian MCH1 receptor.

This invention provides a process involving competitive binding for identifying a chemical compound which specifically binds to a mammalian MCH1 receptor which comprises separately contacting cells expressing on their cell surface the mammalian MCH1 receptor, wherein such cells do not normally express the mammalian MCH1 receptor, with both the chemical compound and a second chemical compound known to bind to the receptor, and with only the second chemical compound, under conditions suitable for binding of both compounds, and detecting specific binding of the chemical compound to the mammalian MCH1 receptor, a decrease in the binding of the second chemical compound to the mammalian MCH1 receptor in the presence of the chemical compound indicating that the chemical compound binds to the mammalian MCH1 receptor.

This invention provides a process involving competitive binding for identifying a chemical compound which specifically binds to a mammalian MCH1 receptor which comprises separately contacting a membrane fraction from a cell extract of cells expressing on their cell surface the mammalian MCH1 receptor, wherein such cells do not normally express the mammalian MCH1 receptor, with both the chemical compound and a second chemical compound known to bind to the receptor, and with only the second chemical compound, under conditions suitable for binding of both compounds, and detecting specific binding of the chemical compound to the mammalian MCH1 receptor, a decrease in the binding of the second chemical compound to the mammalian MCH1 receptor in the presence of the chemical compound indicating that the chemical compound binds to the mammalian MCH1 receptor.

This invention provides a method of screening a plurality of chemical compounds not known to bind to a mammalian MCH1 receptor to identify a compound which specifically binds to the mammalian MCH1 receptor, which comprises (a) contacting cells transfected with and expressing DNA encoding the mammalian MCH1 receptor with a compound known to bind specifically to the mammalian MCH1 receptor; (b) contacting the preparation of step (a) with the plurality of compounds not known to bind specifically to the mammalian MCH1 receptor, under conditions permitting binding of compounds known to bind the mammalian MCH1 receptor; (c) determining whether the binding of the compound known to bind to the mammalian MCH1 receptor is reduced in the presence of the compounds within the plurality of compounds, relative to the binding of the compound in the absence of the plurality of compounds; and if so (d) separately determining the binding to the mammalian MCH1 receptor of compounds included in the plurality of compounds, so as to thereby identify the compound which specifically binds to the mammalian MCH1 receptor.

This invention provides a method of screening a plurality of chemical compounds not known to bind to a mammalian MCH1 receptor to identify a compound which specifically binds to the mammalian MCH1 receptor, which comprises (a) contacting a membrane preparation from cells transfected with and expressing DNA encoding a mammalian MCH1 receptor with a compound known to bind specifically to the mammalian MCH1 receptor; (b) contacting the preparation of step (a) with the plurality of compounds not known to bind specifically to the mammalian MCH1 receptor, under conditions permitting binding of compounds known to bind the mammalian MCH1 receptor; (c) determining whether the binding of the compound known to bind to the mammalian MCH1 receptor is reduced in the presence of the compounds within the plurality of compounds, relative to the binding of the compound in the absence of the plurality of compounds; and if so (d) separately determining the binding to the mammalian MCH1 receptor of compounds included in the plurality of compounds, so as to thereby identify the compound which specifically binds to the mammalian MCH1 receptor.

This invention provides a method of detecting expression of a mammalian MCH1 receptor by detecting the presence of mRNA coding for the mammalian MCH1 receptor which comprises obtaining total mRNA from the cell and contacting the mRNA so obtained with a nucleic acid probe under hybridizing conditions, detecting the presence of mRNA hybridizing to the probe, and thereby detecting the expression of the mammalian MCH1 receptor by the cell.

This invention provides a method of detecting the presence of a mammalian MCH1 receptor on the surface of a cell which comprises contacting the cell with an antibody under conditions permitting binding of the antibody to the receptor, detecting the presence of the antibody bound to the cell, and thereby detecting the presence of the mammalian MCH1 receptor on the surface of the cell.

This invention provides a method of determining the physiological effects of varying levels of activity of human MCH1 receptors which comprises producing a transgenic, nonhuman mammal whose levels of human MCH1 receptor activity are varied by use of an inducible promoter which regulates human MCH1 receptor expression.

This invention provides a method of determining the physiological effects of varying levels of activity of human MCH1 receptors which comprises producing a panel of transgenic, nonhuman mammals each expressing a different amount of human MCH1 receptor.

This invention provides a method for identifying an antagonist capable of alleviating an abnormality wherein the abnormality is alleviated by decreasing the activity of a human MCH1 receptor comprising administering a compound to the transgenic, nonhuman mammal and determining whether the compound alleviates the physical and behavioral abnormalities displayed by the transgenic, nonhuman mammal as a result of overactivity of a human MCH1 receptor, the alleviation of the abnormality identifying the compound as an antagonist. This invention also provides an antagonist identified by this method. This invention further provides a pharmaceutical composition comprising an antagonist identified by this method and a pharmaceutically acceptable carrier.

This invention provides a method of treating an abnormality in a subject wherein the abnormality is alleviated by decreasing the activity of a human MCH1 receptor which comprises administering to the subject an effective amount of this pharmaceutical composition, thereby treating the abnormality.

This invention provides a method for identifying an agonist capable of alleviating an abnormality in a subject wherein the abnormality is alleviated by increasing the activity of a human MCH1 receptor comprising administering a compound to a transgenic, nonhuman mammal, and determining whether the compound alleviates the physical and behavioral abnormalities displayed by the transgenic, nonhuman mammal, the alleviation of the abnormality identifying the compound as an agonist. This invention also provides an agonist identified by this method. This invention further provides a pharmaceutical composition comprising an agonist identified by this method and a pharmaceutically acceptable carrier. This invention provides a method of treating an abnormality in a subject wherein the abnormality is alleviated by increasing the activity of a human MCH1 receptor which comprises administering to the subject an effective amount of this pharmaceutical composition, thereby treating the abnormality.

This invention provides a method for diagnosing a predisposition to a disorder associated with the activity of a specific mammalian allele which comprises: (a) obtaining DNA of subjects suffering from the disorder; (b) performing a restriction digest of the DNA with a panel of restriction enzymes; (c) electrophoretically separating the resulting DNA fragments on a sizing gel; (d) contacting the resulting gel with a nucleic acid probe capable of specifically hybridizing with a unique sequence included within the sequence of a nucleic acid molecule encoding a human MCH1 receptor and labeled with a detectable marker; (e) detecting labeled bands which have hybridized to the DNA encoding a human MCH1 receptor labeled with a detectable marker to create a unique band pattern specific to the DNA of subjects suffering from the disorder; (f) preparing DNA obtained for diagnosis by steps (a)-(e); and (g) comparing the unique band pattern specific to the DNA of subjects suffering from the disorder from step (e) and the DNA obtained for diagnosis from step (f) to determine whether the patterns are the same or different and to diagnose thereby predisposition to the disorder if the patterns are the same.

This invention provides a method of preparing a purified human MCH1 receptor which comprises: (a) inducing cells to express the human MCH1 receptor; (b) recovering the human MCH1 receptor from the induced cells; and (c) purifying the human MCH1 receptor so recovered.

This invention provides a method of preparing a purified human MCH1 receptor which comprises: (a)inserting nucleic acid encoding the human MCH1 receptor in a suitable vector; (b) introducing the resulting vector in a suitable host cell; (c) placing the resulting cell in suitable condition permitting the production of the isolated human MCH1 receptor; (d) recovering the human MCH1 receptor produced by the resulting cell; and (e) purifying the human MCH1 receptor so recovered.

This invention provides a process for determining whether a chemical compound is a mammalian MCH1 receptor agonist which comprises contacting cells transfected with and expressing DNA encoding the mammalian MCH1 receptor with the compound under conditions permitting the activation of the mammalian MCH1 receptor, and detecting an increase in mammalian MCH1 receptor activity, so as to thereby determine whether the compound is a mammalian MCH1 receptor agonist. This invention also provides a pharmaceutical composition which comprises an amount of a mammalian MCH1 receptor agonist determined by this process effective to increase activity of a mammalian MCH1 receptor and a pharmaceutically acceptable carrier.

This invention provides a process for determining whether a chemical compound is a mammalian MCH1 receptor antagonist which comprises contacting cells transfected with and expressing DNA encoding the mammalian MCH1 receptor with the compound in the presence of a known mammalian MCH1 receptor agonist, under conditions permitting the activation of the mammalian MCH1 receptor, and detecting a decrease in mammalian MCH1 receptor activity, so as to thereby determine whether the compound is a mammalian MCH1 receptor antagonist. This invention also provides a pharmaceutical composition which comprises an amount of a mammalian MCH1 receptor antagonist determined by this process effective to reduce activity of a mammalian MCH1 receptor and a pharmaceutically acceptable carrier.

This invention provides a process for determining whether a chemical compound specifically binds to and activates a mammalian MCH1 receptor, which comprises contacting cells producing a second messenger response and expressing on their cell surface the mammalian MCH1 receptor, wherein such cells do not normally express the mammalian MCH1 receptor, with the chemical compound under conditions suitable for activation of the mammalian MCH1 receptor, and measuring the second messenger response in the presence and in the absence of the chemical compound, a change in the second messenger response in the presence of the chemical compound indicating that the compound activates the mammalian MCH1 receptor. This invention also provides a compound determined by this process. This invention further provides a pharmaceutical composition which comprises an amount of the compound (a MCH1 receptor agonist) determined by this process effective to increase activity of a mammalian MCH1 receptor and a pharmaceutically acceptable carrier.

This invention provides a process for determining whether a chemical compound specifically binds to and inhibits activation of a mammalian MCH1 receptor, which comprises separately contacting cells producing a second messenger response and expressing on their cell surface the mammalian MCH1 receptor, wherein such cells do not normally express the mammalian MCH1 receptor, with both the chemical compound and a second chemical compound known to activate the mammalian MCH1 receptor, and with only the second chemical compound, under conditions suitable for activation of the mammalian MCH1 receptor, and measuring the second messenger response in the presence of only the second chemical compound and in the presence of both the second chemical compound and the chemical compound, a smaller change in the second messenger response in the presence of both the chemical compound and the second chemical compound than in the presence of only the second chemical compound indicating that the chemical compound inhibits activation of the mammalian MCH1 receptor. This invention also provides a compound determined by this process. This invention further provides a pharmaceutical composition which comprises an amount of the compound (a mammalian MCH1 receptor antagonist) determined by this effective to reduce activity of a mammalian MCH1 receptor and a pharmaceutically acceptable carrier.

This invention provides a method of screening a plurality of chemical compounds not known to activate a mammalian MCH1 receptor to identify a compound which activates the mammalian MCH1 receptor which comprises: (a) contacting cells transfected with and expressing the mammalian MCH1 receptor with the plurality of compounds not known to activate the mammalian MCH1 receptor, under conditions permitting activation of the mammalian MCH1 receptor; (b) determining whether the activity of the mammalian MCH1 receptor is increased in the presence of the compounds; and if so (c) separately determining whether the activation of the mammalian MCH1 receptor is increased by each compound included in the plurality of compounds, so as to thereby identify the compound which activates the mammalian MCH1 receptor. This invention also provides a compound identified by this method. This invention further provides a pharmaceutical composition which comprises an amount of the compound (a mammalian MCH1 receptor agonist) identified by this method effective to increase activity of a mammalian MCH1 receptor and a pharmaceutically acceptable carrier.

This invention provides a method of screening a plurality of chemical compounds not known to inhibit the activation of a mammalian MCH1 receptor to identify a compound which inhibits the activation of the mammalian MCH1 receptor, which comprises: (a) contacting cells transfected with and expressing the mammalian MCH1 receptor with the plurality of compounds in the presence of a known mammalian MCH1 receptor agonist, under conditions permitting activation of the mammalian MCH1 receptor; (b) determining whether the activation of the mammalian MCH1 receptor is reduced in the presence of the plurality of compounds, relative to the activation of the mammalian MCH1 receptor in the absence of the plurality of compounds; and if so (c) separately determining the inhibition of activation of the mammalian MCH1 receptor for each compound included in the plurality of compounds, so as to thereby identify the compound which inhibits the activation of the mammalian MCH1 receptor. This invention also provides a compound identified by this method. This invention further provides a pharmaceutical composition which comprises an amount of the compound (a mammalian MCH1 receptor antagonist) identified by this process effective to decrease activity of a mammalian MCH1 receptor and a pharmaceutically acceptable carrier.

This invention provides a method of treating an abnormality in a subject wherein the abnormality is alleviated by increasing the activity of a mammalian MCH1 receptor which comprises administering to the subject an amount of a compound which is a mammalian MCH1 receptor agonist effective to treat the abnormality.

This invention provides a method of treating an abnormality in a subject wherein the abnormality is alleviated by decreasing the activity of a mammalian MCH1 receptor which comprises administering to the subject an amount of a compound which is a mammalian MCH1 receptor antagonist effective to treat the abnormality.

This invention provides a process for making a composition of matter which specifically binds to a mammalian MCH1 receptor which comprises identifying a chemical compound using any of the processes described herein for identifying a compound which binds to and/or activates or inhibits activation of a mammalian MCH1 receptor and then synthesizing the chemical compound or a novel structural and functional analog or homolog thereof. This invention further provides a process for preparing a pharmaceutical composition which comprises administering a pharmaceutically acceptable carrier and a pharmaceutically acceptable amount of a chemical compound identified by any of the processes described herein for identifying a compound which binds to and/or activates or inhibits activation of a mammalian MCH1 receptor or a novel structural and functional analog or homolog thereof.

This invention provides a process for determining whether a chemical compound is a human MCH1 receptor antagonist which comprises contacting cells transfected with and expressing DNA encoding the human MCH1 receptor with the compound in the presence of a known human MCH1 receptor agonist, under conditions permitting the activation of the human MCH1 receptor, and detecting a decrease in human MCH1 receptor activity, so as to thereby determine whether the compound is a human MCH1 receptor antagonist, wherein the DNA encoding the human MCH1 receptor comprises the sequence shown in FIG. 1 (Seq. ID No. 1) or contained in plasmid pEXJ.HR-TL231 (ATCC Accession No. 203197), the known human MCH1 receptor agonist is MCH or a homolog or analog of MCH, and the cells do not express the MCH1 receptor prior to transfecting them.

This invention also provides a process for determining whether a chemical compound specifically binds to and inhibits activation of a human MCH1 receptor, which comprises separately contacting cells expressing on their cell surface the human MCH1 receptor and producing a second messenger response upon activation of the human MCH1 receptor, wherein such cells do not normally express the human MCH1 receptor and the DNA encoding the human MCH1 receptor comprises the sequence shown in FIG. 1 (Seq. ID No. 1) or contained in plasmid pEXJ.HR-TL231 (ATCC Accession No. 203197), with both the chemical compound and a second chemical compound known to activate the human MCH1 receptor, and with only the second chemical compound, under conditions suitable for activation of the human MCH1 receptor, and measuring the second messenger response in the presence of only the second chemical compound and in the presence of both the second chemical compound and the chemical compound, a smaller change in the second messenger response in the presence of both the chemical compound and the second chemical compound than in the presence of only the second chemical compound indicating that the chemical compound inhibits activation of the human MCH1 receptor, wherein the second chemical compound is MCH or a homolog or analog of MCH.

This invention further provides a method of screening a plurality of chemical compounds not known to inhibit the activation of a human MCH1 receptor to identify a compound which inhibits the activation of the human MCH1 receptor, which comprises:

(a) contacting cells transfected with and expressing the human MCH1 receptor, wherein such cells do not normally express the human MCH1 receptor and the DNA encoding the human MCH1 receptor comprises the sequence shown in FIG. 1 (Seq. ID No. 1) or contained in plasmid pEXJ.HR-TL231 (ATCC Accession No. 203197), with the plurality of compounds in the presence of a known human MCH1 receptor agonist, under conditions permitting activation of the human MCH1 receptor, wherein the known MCH1 receptor agonist is MCH or a homolog or analog of MCH;

(b) determining whether the activation of the human MCH1 receptor is reduced in the presence of the plurality of compounds, relative to the activation of the human MCH1 receptor in the absence of the plurality of compounds; and if so

(c) separately determining the extent of inhibition of activation of the human MCH1 receptor for each compound included in the plurality of compounds, so as to thereby identify the compound which inhibits the activation of the human MCH1 receptor.

This invention provides a process for making a composition of matter which specifically binds to a human MCH1 receptor which comprises identifying a chemical compound which specifically binds to the human MCH1 receptor and then synthesizing the chemical compound or a structural and functional analog or homolog thereof, wherein the chemical compound is identified as binding to the human MCH1 receptor by a process involving competitive binding which comprises contacting cells expressing on their cell surface the human MCH1 receptor, with both the chemical compound and a second chemical compound known to bind to the receptor, and separately with only the second chemical compound, under conditions suitable for binding of both compounds, and detecting the extent of specific binding of the chemical compound to the human MCH1 receptor, a decrease in the binding of the second chemical compound to the human MCH1 receptor in the presence of the chemical compound indicating that the chemical compound binds to the human MCH1 receptor, wherein the cells do not normally express the human MCH1 receptor, the human MCH1 receptor is encoded by nucleic acid comprising the sequence shown in FIG. 1 (Seq. ID No. 1) or contained in plasmid pEXJ.HR-TL231 (ATCC Accession No. 203197), and the second chemical compound is MCH or a homolog or analog of MCH.

This invention further provides a process for making a composition of matter which specifically binds to a human MCH1 receptor which comprises identifying a chemical compound which specifically binds to the human MCH1 receptor and then synthesizing the chemical compound or a structural and functional analog or homolog thereof, wherein the chemical compound is identified as binding to the human MCH1 receptor by a process involving competitive binding which comprises contacting a membrane preparation from cells expressing on their cell surface the human MCH1 receptor, with both the chemical compound and a second chemical compound known to bind to the receptor, and separately with only the second chemical compound, under conditions suitable for binding of both compounds, and detecting the extent of specific binding of the chemical compound to the human MCH1 receptor, a decrease in the binding of the second chemical compound to the human MCH1 receptor in the presence of the chemical compound indicating that the chemical compound binds to the human MCH1 receptor, wherein the cells do not normally express the human MCH1 receptor, the human MCH1 receptor is encoded by nucleic acid comprising the sequence shown in FIG. 1 (Seq. ID No. 1) or contained in plasmid PEXJ.HR-TL231 (ATCC Accession No. 203197), and the second chemical compound is MCH or a homolog or analog of MCH.

This invention also provides a process for making a composition of matter which is a human MCH1 receptor antagonist which comprises identifying a chemical compound which is a human MCH1 receptor antagonist and then synthesizing the chemical compound or a structural and functional analog or homolog thereof, wherein the chemical compound is identified as a human MCH1 receptor antagonist by a process which comprises contacting cells transfected with and expressing DNA encoding the human MCH1 receptor with the compound in the presence of a known human MCH1 receptor agonist, under conditions permitting the activation of the human MCH1 receptor, and detecting a decrease in human MCH1 receptor activity, so as to thereby determine whether the compound is a human MCH1 receptor antagonist, wherein the cells do not normally express the human MCH1 receptor, the human MCH1 receptor is encoded by nucleic acid comprising the sequence shown in FIG. 1 (Seq. ID No. 1) or contained in plasmid pEXJ.HR-TL231 (ATCC Accession No. 203197), and the known human MCH1receptor agonist is MCH or a homolog or analog of MCH.

This inventions still further provides a process for making a composition of matter which specifically binds to and inhibits the activation of a human MCH1 receptor which comprises identifying a chemical compound which specifically binds to and inhibits the activation of the human MCH1 receptor and then synthesizing the chemical compound or a structural and functional analog or homolog thereof, wherein the chemical compound is identified as binding to and inhibiting the activation of the human MCH1 receptor by a process which comprises separately contacting cells expressing on their cell surface the human MCH1 receptor and producing a second messenger response upon activation of the human MCH1 receptor, wherein such cells do not normally express the human MCH1 receptor and the human MCH1 receptor is encoded by nucleic acid comprising the sequence shown in FIG. 1 (Seq. ID No. 1) or contained in plasmid pEXJ.HR-TL231 (ATCC Accession No. 203197), with both the chemical compound and a second chemical compound known to activate the human MCH1 receptor, and with only the second chemical compound, under conditions suitable for activation of the human MCH1 receptor, and measuring the second messenger response in the presence of only the second chemical compound and in the presence of both the second chemical compound and the chemical compound, a smaller change in the second messenger response in the presence of both the chemical compound and the second chemical compound than in the presence of only the second chemical compound indicating that the chemical compound inhibits activation of the human MCH1 receptor, wherein the second chemical compound is MCH or a homolog or analog of MCH.

This invention provides a process for preparing a composition which comprises identifying a chemical compound which specifically binds to a human MCH1 receptor, and then admixing a carrier and the chemical compound or a structural and functional analog or homolog thereof, wherein the chemical compound is identified as binding to the human MCH1 receptor by a process involving competitive binding which comprises contacting cells expressing on their cell surface the human MCH1 receptor, with both the chemical compound and a second chemical compound known to bind to the receptor, and separately with only the second chemical compound, under conditions suitable for binding of both compounds, and detecting the extent of specific binding of the chemical compound to the human MCH1 receptor, a decrease in the binding of the second chemical compound to the human MCH1 receptor in the presence of the chemical compound indicating that the chemical compound binds to the human MCH1 receptor, wherein the cells do not normally express the human MCH1 receptor, the human MCH1 receptor is encoded by nucleic acid comprising the sequence shown in FIG. 1 (Seq. ID No. 1) or contained in plasmid pEXJ.HR-TL231 (ATCC Accession No. 203197), and the second chemical compound is MCH or a homolog or analog of MCH.

This invention further provides a process for preparing a composition which comprises identifying a chemical compound which specifically binds to a human MCH1 receptor, and then admixing a carrier and the chemical compound or a structural and functional analog or homolog thereof, wherein the chemical compound is identified as binding to the human MCH1 receptor by a process involving competitive binding which comprises contacting a membrane preparation from cells expressing on their cell surface the human MCH1 receptor, with both the chemical compound and a second chemical compound known to bind to the receptor, and separately with only the second chemical compound, under conditions suitable for binding of both compounds, and detecting the extent of specific binding of the chemical compound to the human MCH1 receptor, a decrease in the binding of the second chemical compound to the human MCH1 receptor in the presence of the chemical compound indicating that the chemical compound binds to the human MCH1 receptor, wherein the cells do not normally express the human MCH1 receptor, the human MCH1 receptor is encoded by nucleic acid comprising the sequence shown in FIG. 1 (Seq. ID No. 1) or contained in plasmid pEXJ.HR-TL231 (ATCC Accession No. 203197), and the second chemical compound is MCH or a homolog or analog of MCH.

This invention also provides a process for preparing a composition which comprises identifying a chemical compound which is a human MCH1 receptor antagonist, and then admixing a carrier and the chemical compound or a structural and functional analog or homolog thereof, wherein the chemical compound is identified as a human MCH1 receptor antagonist by a process which comprises contacting cells transfected with and expressing DNA encoding the human MCH1 receptor with the compound in the presence of a known human MCH1 receptor agonist, under conditions permitting the activation of the human MCH1 receptor, and detecting a decrease in human MCH1 receptor activity, so as to thereby determine whether the compound is a human MCH1 receptor antagonist, wherein the cells do not normally express the human MCH1 receptor, the human MCH1 receptor is encoded by nucleic acid comprising the sequence shown in FIG. 1 (Seq. ID No. 1) or contained in plasmid pEXJ.HR-TL231 (ATCC Accession No. 203197), and the known human MCH1 receptor agonist is MCH or a homolog or analog of MCH.

This invention still further provides a process for preparing a composition which comprises identifying a chemical compound which specifically binds to and inhibits the activation of a human MCH1 receptor, and then admixing a carrier and the chemical compound or a structural and functional analog or homolog thereof, wherein the chemical compound is identified as binding to and inhibiting activation of the human MCH1 receptor by a process which comprises separately contacting cells expressing on their cell surface the human MCH1 receptor and producing a second messenger response upon activation of the human MCH1 receptor, wherein such cells do not normally express the human MCH1 receptor and the human MCH1 receptor is encoded by nucleic acid comprising the sequence shown in FIG. 1 (Seq. ID No. 1) or contained in plasmid pEXJ.HR-TL231 (ATCC Accession No. 203197), with both the chemical compound and a second chemical compound known to activate the human MCH1 receptor, and with only the second chemical compound, under conditions suitable for activation of the human MCH1 receptor, and measuring the second messenger response in the presence of only the second chemical compound and in the presence of both the second chemical compound and the chemical compound, a smaller change in the second messenger response in the presence of both the chemical compound and the second chemical compound than in the presence of only the second chemical compound indicating that the chemical compound inhibits activation of the human MCH1 receptor, wherein the second chemical compound is MCH or a homolog or analog of MCH.

This invention provides a method of treating an eating disorder or obesity in a subject which comprises administering to the subject a therapeutically effective amount of an MCH1 antagonist which inhibits the activation of the MCH1 receptor.

This invention provides a method of reducing the body mass of a subject which comprises administering to the subject an amount of an MCH1 antagonist effective to reduce the body mass of the subject.

This invention further provides a method of treating an eating disorder in a subject which comprises administering to the subject a therapeutically effective amount of an MCH1 agonist which activates the MCH1 receptor.

This invention also provides a method of treating depression and/or anxiety in a subject which comprises administering to the subject a composition comprising a pharmaceutically acceptable carrier and a therapeutically effective amount of a MCH1 receptor antagonist, wherein:

(a) (1) the MCH1 receptor antagonist does not inhibit the activity of central monoamine oxidase A greater than 50 percent, at a concentration of 10 mM; and (2) the MCH1 receptor antagonist does not inhibit the activity of central monoamine oxidase B greater than 50 percent, at a concentration of 10 mM; and

(b) the MCH1 receptor antagonist binds to the human MCH1 receptor with a binding affinity at least ten-fold higher than the binding affinity with which it binds to each of the following transporters: serotonin transporter, norepinephrine transporter, and dopamine transporter.

### BRIEF DESCRIPTION OF THE FIGURES

FIG. 1

Nucleotide sequence encoding a human MCH1 receptor (MCH1) (SEQ ID NO: 1). Three potential start (ATG) codons and the stop (TGA) codon are underlined.

FIG. 2

Deduced amino acid sequence (SEQ ID NO: 2) of the human MCH1 receptor (MCH1) encoded by the nucleotide sequence shown FIG. 1 (SEQ ID NO: 1).

FIG. 3

Deduced amino acid sequence for human MCH1 (SEQ ID NO: 2).

The seven putative transmembrane (TM) regions are underlined.

FIG. 4

Nucleotide sequence of rat MCH1 (SEQ ID NO: 3). One start (ATG) codon and the stop codon (TGA) are underlined.

FIG. 5

Deduced amino acid sequence for rat MCH1 (SEQ ID NO: 4).

FIG. 6

MCH1-mediated PI dose response to MCH.

FIG. 7

MCH1 challenge with several compounds of interest.

FIG. 8

MCH1-mediated extracellular acidification response to MCH and Phe¹³,Tyr¹⁹-MCH. Results are reported as the average of two independent experiments performed in duplicate.

FIG. 9

Transcriptional response of MCH1-transfected Cos-7 cells to MCH.

FIG. 10

Binding of [¹²⁵I]Phe¹³,Tyr¹⁹-MCH on MCH1-transfected Cos-7 cell membranes. Results are means ±S.E.M. (vertical lines) of triplicate determinations.

FIG. 11

RT-PCR detection of MCH1 receptor mRNA in human mRNA samples.

FIG. 12

Amino acid alignment of the N-terminal regions of MCH1 receptors encoded by various plasmids. The mutations present in R106 (SEQ ID NO: 16) and R114 (SEQ ID NO: 17) are shown in lower case. Potential initiating methionines are shown in bold. The amino acid sequence downstream of position 100 is identical for all four plasmids.

FIG. 13

Amino acid sequence (SEQ ID NO: 26) of the mutant human MCH1 receptor encoded by plasmid R106.

FIG. 14

Amino acid sequence (SEQ ID NO: 27) of the mutant human MCH1 receptor encoded by plasmid R114.

FIG. 15

Amino acid sequence (SEQ ID NO: 28) of the mutant human MCH1 receptor encoded by plasmid BO120.

FIG. 16

Antagonism by Compound 10 shown by the phosphoinositide response induced by MCH in Cos-7 cells transfected with MCH1. Inset: Schild plot, y axis=((EC50_{MCH+Cmpd10}/EC50_{MCH})−1); x axis=Log (Cmpd10) [M] . The analysis by linear regression analysis estimated a pA2 (x-intercept)=9.24, slope=0.97+0.2 and r²=0.94.

FIG. 17

Saturation equilibrium binding of [3H]Compound 10 to the human MCH1 receptor. Membrane preparations from Cos-7 cells transfected with MCH1 were incubated with varying concentrations of [3H]Compound 10 (SA: 56 Ci/mmol) at room temperature for 90 min, in a volume of 0.250 ml. The reaction was terminated by filtration in GF/C filters, and the radioactivity determined by scintillation counting. Non-specific binding was defined as the amount of radioactivity retained in the filter after incubating the reaction mixture in the presence of unlabeled Compound 10 (10 mM).

FIG. 18

Competition binding of [3H]Compound 10 to the human MCH1 receptor. Membrane preparations from Cos-7 cells transfected with MCH1 were incubated with 0.4 nM [3H]Compound in the presence of varying concentrations of MCH (from 1E-11 to 1E-6 M) or unlabeled Compound 10 (from 1E-10 to 1E-5 M), for 90 min at room temperature. The reaction was terminated by filtration in GF/C filters and the radioactivity bound to the membrane was determined by scintillation counting.

FIG. 19

Autoradiographic localization of MCH1 receptor binding sites in the rat diencephalon. A. Total MCH1 receptor binding obtained with 0.1 nM [³H]Compound 10 in the presence of 1 μM prazosin and 100 μM dopamine. B. Nonspecific binding observed in the presence of 1 μM cold Compound 10.

FIGS. 20A and 20B

Autoradiographic distribution of MCH1 binding sites using [³H]Compound 10 in the presence of 1 μM prazosin and 100 μM dopamine in the rat CNS presented rostrocaudally. Coronal rat brain sections at the level of the frontal cortex (A), the forebrain/basal ganglia (B), the basal ganglia (C), the diencephalon (D-H), the midbrain (I-J), the brain stem (K-L), and transverse through the lumbar spinal cord (M). Note the dense labeling of several brain regions such as the caudate-putamen (CPu) and accumbens nucleus (AcbSh and AcbC) (B). Moderate labeling was observed in the hippocampus (E-H), subthalamic nucleus (F) and locus coeruleus (L) while weaker labeling is seen in the thalamus and hypothalamus (D-H).

List of Abbreviations

| | |
|---|---|
| AAV | anterior amygdaloid area, ventral |
| AcbC | accumbens nucleus, core |
| AcbSh | accumbens nucleus, core |
| ACo | anterior cortical amygdaloid nucleus |
| AD | anterodorsal thalamic nucleus |
| AH | anterior hypothalamus |
| AI | agranular insular cortex |
| Arc | arcuate hypothalamic nucleus |
| AON | anterior olfactory nucleus |
| AU | auditory cortex |
| AV | anteroventral hypothalamic nucleus |
| BLA | basolateral amygdaloid nucleus |
| BSTM | bed nucleus of the stria terminalis, medial |
| | div. |
| CA1, 2, 3 | fields CAl, 2, 3 of hippocampus |
| Cg | cingulate cortex |
| CL | claustrum |
| CPu | caudate-putamen |
| DLG | dorsal lateral geniculate |
| DM | dorsomedial hypothalamic nucleus |
| DR | dorsal raphe nucleus |
| DTN | dorsal tegmental nucleus |
| Ent | entorhinal cortex |
| GP | globus pallidus |
| IAM | interanteromedial thalamic nucleus |
| IC | inferior colliculus |
| ICjM | islands of Calleja, major island |
| IG | indusium griseum |
| La | lateral amygdaloid nucleus |
| LC | locus coeruleus |
| LD | laterodorsal thalamic nucleus |
| LH | lateral hypothalamic area |
| LO | lateral preoptic area |
| LSD | lateral septal nucleus, dorsal part |
| LSO | lateral superior olive |
| M1 | primary motor cortex |
| Me | medial amygdaloid nucleus |
| MG | medial geniculate nucleus |
| MHb | medial habenular nucleus |
| MM | medial mammillary nucleus |
| MPO | medial preoptic area |
| OC | occipital cortex |
| PAG | periaqueductal gray |
| PB | parabrachial nucleus |
| PF | parafascicular thalamic nucleus |
| PH | posterior hypothalamic area |
| Pir | piriform cortex |
| PMCo | posteromedial amygdaloid nucleus |
| Pn | pontine nuclei |
| Po | posterior thalamic nuclear group |
| PVA | paraventricular thalamic nucleus |
| PVP | paraventricular thalamic nucleus, posterior |
| RSG | retrosplenial granular cortex |
| SC | superior colliculus |
| SNR | substantia nigra, reticular part |
| STh | subthalamic nucleus |
| S1 | primary somatosensory cortex |
| so | stratum oriens field CAI |
| sr | stratum radiatum field CAI |
| Tu | olfactory tubercle |
| V2 | secondary visual cortex |
| VL | ventrolateral thalamic nucleus |
| VMH | ventromedial hypothalamic nucleus |
| VP | ventroposterior thalamic nucleus |

FIG. 21

Effect of Compound 10 on MCH-induced stimulation of food intake in rats. MCH (3 nmol) or vehicle was administered into the third venticle, and food intake measured 30, 60 and 120 minutes later. Some rats were pretreated with vehicle or Compound 10 (1 or 10 mg/kg) i.p. 20 minutes prior to i.c.v. injection.

* Significantly greater than vehicle, +significantly less than vehicle /MCH.

FIG. 22

Effect of Compound 10 on body weight gain in young growing rats. Compound 10 (10 mg/kg/day), fenfluramine (6 mg/kg/day) or vehicle were administered to rats for 14 days via subcutaneously implanted osmotic minipumps. Significant differences from vehicle are denoted by **P<0.001, *P<0.01, xP<0.05, as determined by ANOVA and Newman-Keuls test.

FIG. 23

Effect of Compound 10 on body weight gain in young growing rats. Compound 10 (1, 3 or 10 mg/kg) or vehicle (dashed line) was administered to rats twice daily by i.p. injection. Significant differences from vehicle are denoted by **P<0.001, *P<0.01, as determined by ANOVA and Newman-Keuls test.

FIG. 24

Effect of Compound 94 on body weight gain in young growing rats. Compound 94 (3, 10 or 30 mg/kg) or vehicle was administered to rats twice daily by i.p. injection. Significant differences from vehicle are denoted by +P<0.05, *P<0.01, as determined by ANOVA and Newman-Keuls test.

FIG. 25

Effect of Compound 95 on body weight gain in young growing rats. Compound 67173 (3, 10 or 30 mg/kg) or vehicle was administered to rats twice daily by i.p. injection. Significant differences from vehicle are denoted by *P<0.001, as determined by ANOVA and Newman-Keuls test.

FIG. 26

Effect of Compound 10 on sweetened condensed milk consumption in rats. Rats were trained to drink sweetened condensed milk for 20 minutes a day. On the test day, Compound 10 (3, 10 or 30 mg/kg), fenfluramine (3 mg/kg) or vehicle was administered i.p. 30 minutes prior to milk exposure. Significant differences from vehicle are denoted by *P<0.05, **P<0.001 as determined by two-tailed t-test.

### DETAILED DESCRIPTION OF THE INVENTION

Throughout this application, the following standard abbreviations are used to indicate specific nucleotide bases:

A=adenine

G=guanine

C=cytosine

T=thymine

U=uracil

M=adenine or cytosine

R=adenine or guanine

W=adenine, thymine, or uracil

S=cytosine or guanine

Y=cytosine, thymine, or uracil

K=guanine, thymine, or uracil

V=adenine, cytosine, or guanine (not thymine or uracil

H=adenine, cytosine, thymine, or uracil (not guanine)

D=adenine, guanine, thymine, or uracil (not cytosine)

B=cytosine, guanine, thymine, or uracil (not adenine)

N=adenine, cytosine, guanine, thymine, or uracil (or other modified base such as inosine)

I=inosine

Furthermore, the term “agonist” is used throughout this application to indicate any peptide or non-peptidyl compound which increases the activity of any of the polypeptides of the subject invention. The term “antagonist” is used throughout this application to indicate any peptide or non-peptidyl compound which decreases the activity of any of the polypeptides of the subject invention. The term “mammalian” is used throughout this invention to include mutant forms of the human MCH1 receptor.

The activity of a G-protein coupled receptor such as the polypeptides disclosed herein may be measured using any of a variety of functional assays in which activation of the receptor in question results in an observable change in the level of some second messenger system, including, but not limited to, adenylate cyclase, calcium mobilization, arachidonic acid release, ion channel activity, inositol phospholipid hydrolysis or guanylyl cyclase. Heterologous expression systems utilizing appropriate host cells to express the nucleic acid of the subject invention are used to obtain the desired second messenger coupling. Receptor activity may also be assayed in an oocyte expression system.

In the case that a receptor has activity in the absence of an agonist (constitutive receptor activity) the antagonist may act as an inverse agonist or an allosteric modulator, as opposed to a neutral antagonist, and suppress receptor signaling independent of the agonist (Lutz and Kenakin, 1999). The categories of “antagonist compounds” are therefore seen to include 1) neutral antagonists (which block agonist actions but do not affect constitutive activity); 2) inverse agonists (which block agonist actions as well as constitutive activity by stabilizing an inactive receptor conformation); 3) and allosteric modulators (which block agonist actions to a limited extent and which may also block constitutive activity through allosteric regulation). The probability that an antagonist is neutral and therefore of “zero efficacy” is relatively low, given that this would require identical affinities for different tertiary conformations of the receptor. Thus, Kenakin proposed in 1996 that, “with the development of sensitive test systems for the detection of inverse agqnism will come a reclassification of many drugs . . . it might be observed that numerous previously classified neutral antagonists may be inverse agonists” (Kenakin, 1996). Indeed, there is now evidence from studies with known pharmacological agents to support the existence of inverse agonists for numerous receptors, including histamine, 5HT_{1A}, 5HT_{2C}, cannabinoid, dopamine, calcitonin and human formyl peptide receptors, among others (de Ligt, et al, 2000; Herrick-Davis, et al, 2000; Bakker, et al, 2000). In the case of the 5HT_{2C} receptor, clinically effective atypical antipsychotics drugs such as sertindole, clozapine, olanzapine, ziprasidone, risperidone, zotepine, tiospirone, fluperlapine and tenilapine displayed potent inverse activity whereas typical antipsychotic drugs such as chlorpromazine, thioridazine, spiperone and thiothixene were classified as neutral antagonists (Herrick-Davis et al, 2000). In the case of the histamine H₁ receptor, the therapeutically used anti-allergics cetirizine, loratadine and epinastine were found to be inverse agonists. These findings further extend the idea that many compounds previously thought of as neutral antagonists will be reclassified as inverse agonists when tested in a constitutively active receptor system (de Ligt et al, 2000).

It is possible that the human MCH1 receptor gene contains introns and furthermore, the possibility exists that additional introns could exist in coding or non-coding regions. In addition, spliced form(s) of mRNA may encode additional amino acids either upstream of the currently defined starting methionine or within the coding region. Further, the existence and use of alternative exons is possible, whereby the mRNA may encode different amino acids within the region comprising the exon. In addition, single amino acid substitutions may arise via the mechanism of RNA editing such that the amino acid sequence of the expressed protein is different than that encoded by the original gene. (Burns et al., 1996; Chu et al., 1996). Such variants may exhibit pharmacologic properties differing from the polypeptide encoded by the original gene.

This invention provides splice variants of the human MCH1 receptor disclosed herein. This invention further provides for alternate translation initiation sites and alternately spliced or edited variants of nucleic acids encoding the human MCH1 receptor of this invention.

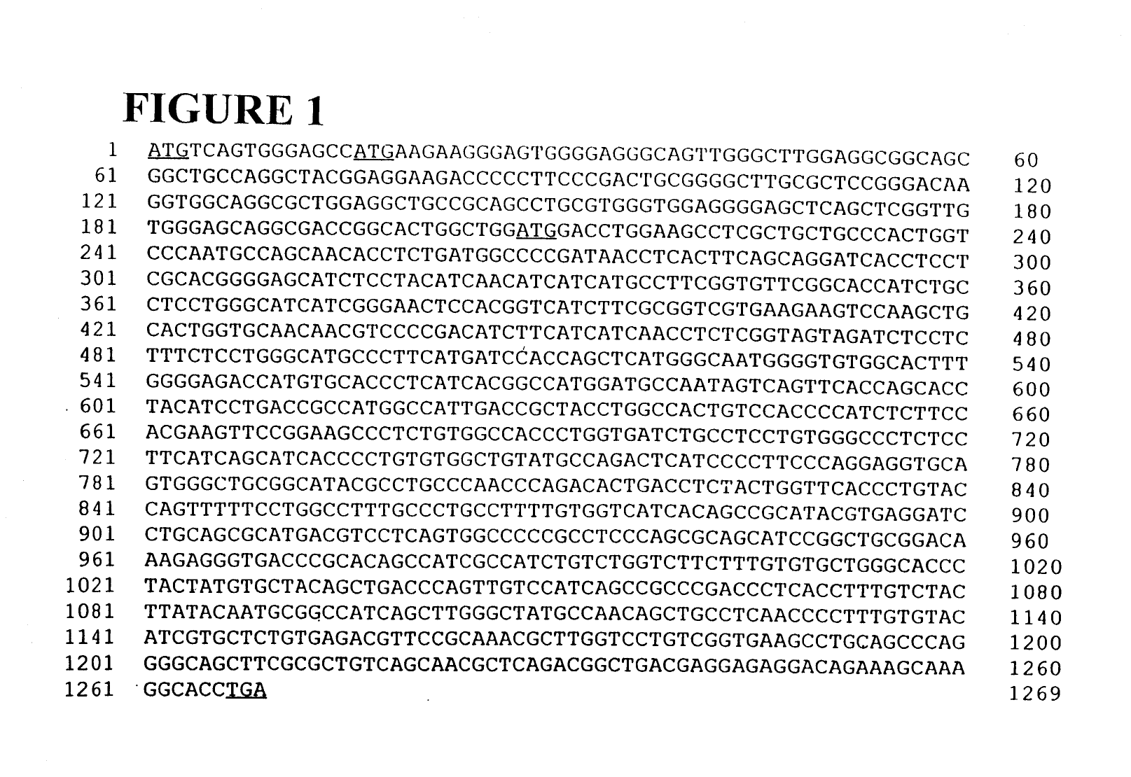

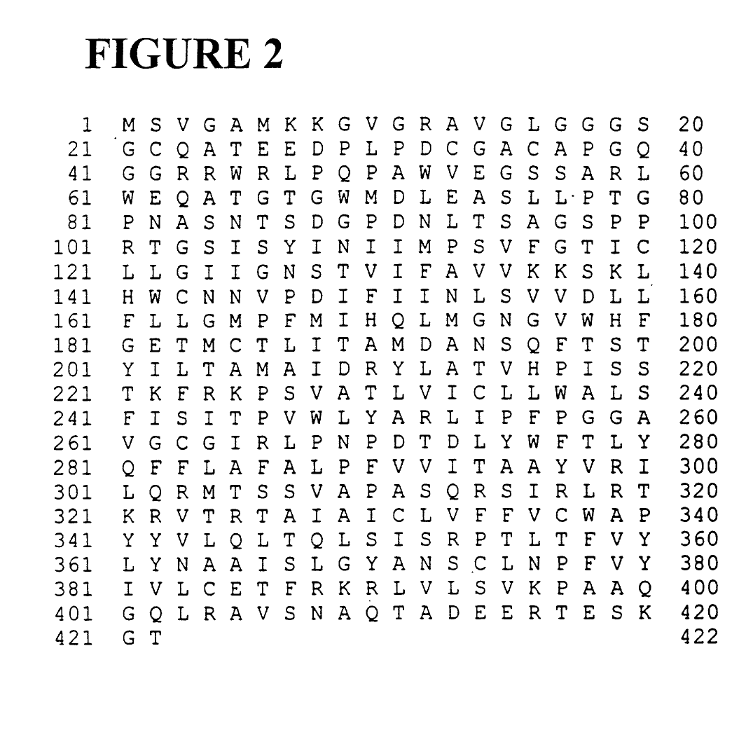

The nucleic acid of the subject invention also includes nucleic acid analogs of the human MCH1 receptor gene, wherein the human MCH1 receptor gene comprises the nucleic acid sequence shown in FIG. 1 or contained in plasmid pEXJ.HR-TL231 (ATCC Accession No. 203197). Nucleic acid analogs of the human MCH1 receptor genes differ from the human MCH1 receptor gene described herein in terms of the identity or location of one or more nucleic acid bases (deletion analogs containing less than all of the nucleic acid bases shown in FIG. 1 or contained in plasmid pEXJ.HR-TL231, substitution analogs wherein one or more nucleic acid bases shown in FIG. 1 or contained in plasmids pEXJ.HR-TL231 are replaced by other nucleic acid bases, and addition analogs, wherein one or more nucleic acid bases are added to a terminal or medial portion of the nucleic acid sequence) and which encode proteins which share some or all of the properties of the proteins encoded by the nucleic acid sequences shown in FIG. 1 or contained in plasmid pEXJ.HR-TL231. In one embodiment of the present invention, the nucleic acid analog encodes a protein which comprises an amino acid sequence as shown in FIG. 2 or encoded by the nucleic acid sequence contained in plasmid pEXJ.HR-TL231. In another embodiment, the nucleic acid analog encodes a protein comprising an amino acid sequence which differs from the amino acid sequences shown in FIG. 2 or encoded by the nucleic acid contained in plasmids pEXJ.HR-TL231. In a further embodiment, the protein encoded by the nucleic acid analog has a function which is the same as the function of the receptor protein comprising the amino acid sequence shown in FIG. 2. In another embodiment, the function of the protein encoded by the nucleic acid analog differs from the function of the receptor protein comprising the amino acid sequence shown in FIG. 2. In another embodiment, the variation in the nucleic acid sequence occurs within the transmembrane (TM) region of the protein. In a further embodiment, the variation in the nucleic acid sequence occurs outside of the TM region.

This invention provides the above-described isolated nucleic acid, wherein the nucleic acid is DNA. In an embodiment, the DNA is cDNA. In another embodiment, the DNA is genomic DNA. In still another embodiment, the nucleic acid is RNA. Methods for production and manipulation of nucleic acid molecules are well known in the art.

This invention further provides nucleic acid which is degenerate with respect to the DNA encoding the polypeptides described herein. In an embodiment, the nucleic acid comprises a nucleotide sequence which is degenerate with respect to the nucleotides sequence shown in FIG. 1 (SEQ ID NO: 2) or the nucleotide sequence contained in the plasmid pEXJ.HR-TL231, that is, a nucleotide sequence which is translated into the same amino acid sequence.

This invention also encompasses DNAs and cDNAs which encode amino acid sequences which differ from those of the polypeptides of this invention, but which should not produce phenotypic changes. Alternately, this invention also encompasses DNAs, cDNAs, and RNAs which hybridize to the DNA, cDNA, and RNA of the subject invention. Hybridization methods are well known to those of skill in the art.

The nucleic acids of the subject invention also include nucleic acid molecules coding for polypeptide analogs, fragments or derivatives of antigenic polypeptides which differ from naturally-occurring forms in terms of the identity or location of one or more amino acid residues (deletion analogs containing less than all of the residues specified for the protein, substitution analogs wherein one or more residues specified are replaced by other residues and addition analogs wherein one or more amino acid residues is added to a terminal or medial portion of the polypeptides) and which share some or all properties of naturally-occurring forms. These molecules include: the incorporation of codons “preferred” for expression by selected non-mammalian hosts; the provision of sites for cleavage by restriction endonuclease enzymes; and the provision of additional initial, terminal or intermediate DNA sequences that facilitate construction of readily expressed vectors. The creation of polypeptide analogs is well known to those of skill in the art (R. F. Spurney et al. (1997); Fong, T. M. et al. (1995); Underwood, D. J. et al. (1994); Graziano, M. P. et al. (1996); Guan X. M. et al. (1995)).

The modified polypeptides of this invention may be transfected into cells either transiently or stably using methods well-known in the art, examples of which are disclosed herein. This invention also provides for binding assays using the modified polypeptides, in which the polypeptide is expressed either transiently or in stable cell lines. This invention further provides a compound identified using a modified polypeptide in a binding assay such as the binding assays described herein.

The nucleic acids described and claimed herein are useful for the information which they provide concerning the amino acid sequence of the polypeptide and as products for the large scale synthesis of the polypeptides by a variety of recombinant techniques. The nucleic acid molecule is useful for generating new cloning and expression vectors, transformed and transfected prokaryotic and eukaryotic host cells, and new and useful methods for cultured growth of such host cells capable of expression of the polypeptide and related products.

This invention provides an isolated nucleic acid encoding a human MCH1 receptor or a mutant of such human MCH1 receptor which is activated by MCH or an analog or homolog thereof. In one embodiment, the nucleic acid is DNA. In another embodiment, the DNA is cDNA. In another embodiment, the DNA is genomic DNA. In another embodiment, the nucleic acid is RNA.

This invention also provides methods of using an isolated nucleic acid encoding species homologs of the MCH1 receptor encoded by the nucleic acid sequence shown in FIG. 1 (SEQ ID NO: 1) or encoded by the plasmid pEXJ.HR-TL231. In one embodiment, the nucleic acid encodes a mammalian MCH1 receptor homolog which has substantially the same amino acid sequence as does the MCH1 receptor encoded by the plasmid pEXJ.HR-TL231. In another embodiment, the nucleic acid encodes a mammalian MCH1 receptor homolog which has above 65% amino acid identity to the MCH1 receptor encoded by the plasmid pEXJ.HR-TL231; preferably above 75% amino acid identity to the MCH1 receptor encoded by the plasmid pEXJ.HR-TL231; more preferably above 85% amino acid identity to the MCH1 receptor encoded by the plasmid pEXJ.HR-TL231; most preferably above 95% amino acid identity to the MCH1 receptor encoded by the plasmid pEXJ.HR-TL231. In another embodiment, the mammalian MCH1 receptor homolog has above 70% nucleic acid identity to the MCH1 receptor gene contained in plasmid pEXJ.HR-TL231; preferably above 80% nucleic acid identity to the MCH1 receptor gene contained in the plasmid pEXJ.HR-TL231; more preferably above 90% nucleic acid identity to the MCH1 receptor gene contained in the plasmid pEXJ.HR-TL231. Examples of methods for isolating and purifying species homologs are described elsewhere (e.g., U.S. Pat. No. 5,602,024, WO94/14957, WO97/26853, WO98/15570).

In a separate embodiment of the present invention, the nucleic acid encodes a MCH1 receptor which has an amino acid sequence identical to that encoded by the plasmid pEXJ.HR-TL231. In a further embodiment, the MCH1 receptor comprises a sequence substantially the same as the amino acid sequence shown in FIG. 2 (SEQ ID NO: 2). In another embodiment, the MCH1 receptor comprises an amino acid sequence as shown in FIG. 2 (SEQ ID NO: 2).

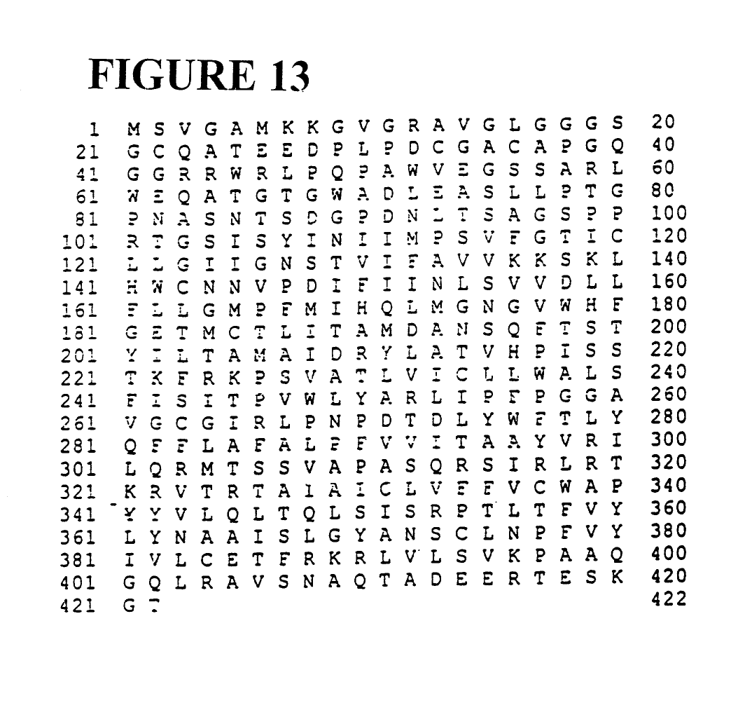

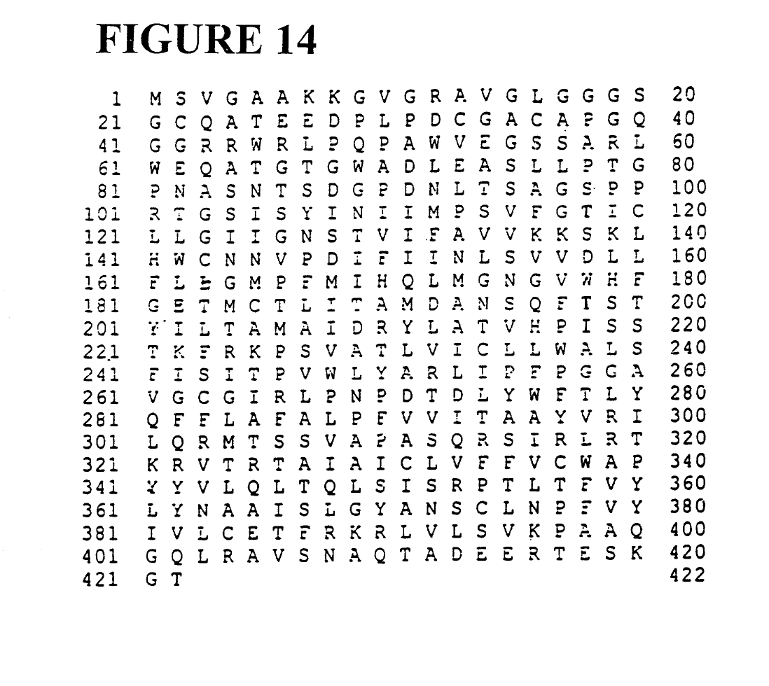

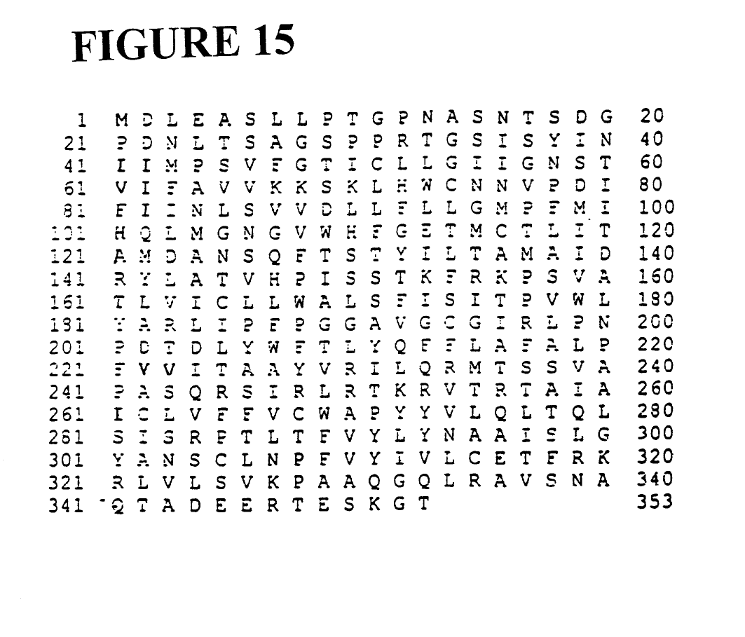

In one embodiment, the mutant human MCH1 receptor comprises an amino acid sequence as shown in FIG. 13 (SEQ ID NO: 26). In another embodiment, the mutant human MCH1 receptor comprises an amino acid sequence as shown in FIG. 14 (SEQ ID NO: 27). In still another embodiment, the mutant human MCH1 receptor comprises an amino acid sequence as shown in FIG. 15 (SEQ ID NO: 28).

In separate embodiments, the human MCH1 receptor is encoded by the nucleic acid sequence shown in FIG. 1 beginning with any of the three indicated start (ATG) codons.

This invention provides an isolated nucleic acid encoding a modified human MCH1 receptor, which differs from a human MCH1 receptor by having an amino acid(s) deletion, replacement, or addition in the third intracellular domain.

This invention provides a nucleic acid encoding a human MCH1 receptor, wherein the nucleic acid (a) hybridizes to a nucleic acid having the defined sequence shown in FIG. 1 (SEQ ID NO: 1) under low stringency conditions or a sequence complementary thereto and (b) is further characterized by its ability to cause a change in the pH of a culture of CHO cells when a MCH1 ligand is added to the culture and the CHO cells contain the nucleic acid which hybridized to the nucleic acid having the defined sequence or its complement. Hybridization at low stringency is performed at 40° C. in a hybridization buffer containing 25% formamide, 5× SCC, 7 mM Tris, 1× Denhardt's, 25 μl/ml salmon sperm DNA. Wash at 40° C. in 0.1× SCC, 0.1% SDS. Changes in pH are measured through microphysiometric measurement of receptor mediated extracellular acidification rates. Because cellular metabolism is intricately involved in a broad range of cellular events (including receptor activation of multiple messenger pathways), the use of microphysiometric measurements of cell metabolism can in principle provide a generic assay of cellular activity arising from the activation of any receptor regardless of the specifics of the receptor's signaling pathway. General guidelines for transient receptor expression, cell preparation and microphysiometric recording are described elsewhere (Salon, J. A. and Owicki, J. A., 1996). Receptors and/or control vectors are transiently expressed in CHO-K1 cells, by liposome mediated transfection according to the manufacturers recommendations (LipofectAMINE, GibcoBRL, Gaithersburg, Md.), and maintained in Ham's F-12 complete (10% serum). A total of lopg of DNA is used to transfect each 75 cm² flask which had been split 24 hours prior to the transfection and judged to be 70-80% confluent at the time of transfection. 24 hours post transfection, the cells are harvested and 3×10⁵ cells seeded into microphysiometer capsules. Cells are allowed to attach to the capsule membrane for an additional 24 hours; during the last 16 hours, the cells are switched to serum-free F-12 complete to minimize ill-defined metabolic stimulation caused by assorted serum factors. On the day of the experiment the cell capsules are transferred to the microphysiometer and allowed to equilibrate in recording media (low buffer RPMI 1640, no bicarbonate, no serum (Molecular Devices Corporation, Sunnyvale, Calif.) containing 0.1% fatty acid free BSA), during which a baseline measurement of basal metabolic activity is established. A standard recording protocol specifies a 100l/min flow rate, with a 2 min total pump cycle which includes a 30 sec flow interruption during which the acidification rate measurement is taken. Ligand challenges involve a 1 min 20 sec exposure to the sample just prior to the first post challenge rate measurement being taken, followed by two additional pump cycles for a total of 5 min 20 sec sample exposure. Typically, drugs in a primary screen are presented to the cells at 10 μM final concentration. Ligand samples are then washed out and the acidification rates reported are expressed as a percentage increase of the peak response over the baseline rate observed just prior to challenge. An examples of a MCH ligand includes, but is not limited to, the endogenous MCH peptide.

This invention provides a purified human MCH1 receptor protein.

This invention provides a vector comprising nucleic acid encoding a human MCH1 receptor. In an embodiment, the vector is adapted for expression in a cell which comprises the regulatory elements necessary for expression of the nucleic acid in the cell operatively linked to the nucleic acid encoding the human MCH1 receptor as to permit expression thereof. In separate embodiments, the cell is a bacterial cell, an amphibian cell, a yeast cell, an insect cell or a mammalian cell. In another embodiment, the vector is a baculovirus. In one embodiment, the vector is a plasmid.

This invention provides a plasmid designated pEXJ.HR-TL231 (ATCC Accession No. 203197). This plasmid comprises the regulatory elements necessary for expression of DNA in a mammalian cell operatively linked to DNA encoding the human MCH1 receptor so as to permit expression thereof.

This plasmid (pEXJ.HR-TL231) was deposited on Sep. 17, 1998, with the American Type Culture Collection (ATCC), 12301 Parklawn Drive, Rockville, Md. 20852, U.S.A. under the provisions of the Budapest Treaty for the International Recognition of the Deposit of Microorganisms for the Purposes of Patent Procedure and was accorded ATCC Accession No. 203197.

This invention further provides for any vector or plasmid which comprises modified untranslated sequences, which are beneficial for expression in desired host cells or for use in binding or functional assays. For example, a vector or plasmid with untranslated sequences of varying lengths may express differing amounts of the polypeptide depending upon the host cell used. In an embodiment, the vector or plasmid comprises the coding sequence of the polypeptide and the regulatory elements necessary for expression in the host cell.

This invention provides a cell comprising a vector comprising a nucleic acid encoding the human MCH1 receptor. In an embodiment, the cell is a non-mammalian cell. In a further embodiment, the non-mammalian cell is a Xenopus oocyte cell or a Xenopus melanophore cell. In another embodiment, the cell is a mammalian cell. In a further embodiment, the mammalian cell is a COS-7 cell, a 293 human embryonic kidney cell, a NIH-3T3 cell, a LM(tk−) cell, a mouse Y1 cell, or a CHO cell.

This invention provides an insect cell comprising a vector adapted for expression in an insect cell which comprises a nucleic acid encoding a human MCH1 receptor. In another embodiment, the insect cell is an Sf9 cell, an Sf21 cell or a Trichoplusia ni 5B1-4 (HighFive) cell.

This invention provides a membrane preparation isolated from any one of the cells described above.

This invention provides a nucleic acid probe comprising at least 15 nucleotides, which probe specifically hybridizes with a nucleic acid encoding a human MCH1 receptor, wherein the probe has a unique sequence corresponding to a sequence present within one of the two strands of the nucleic acid encoding a human MCH1 receptor present in plasmid pEXJ.HR-TL231. This invention also provides a nucleic acid probe comprising at least 15 nucleotides, which probe specifically hybridizes with a nucleic acid encoding a human MCH1 receptor, wherein the probe has a unique sequence corresponding to a sequence present within (a) the nucleic acid sequence shown in FIG. 1 (SEQ ID NO: 1) or (b) the reverse complement thereto. In one embodiment, the nucleic acid is DNA. In another embodiment, the nucleic acid is RNA.

As used herein, the phrase “specifically hybridizing” means the ability of a nucleic acid molecule to recognize a nucleic acid sequence complementary to its own and to form double-helicalsegments through hydrogen bonding between complementary base pairs.

Nucleic acid probe technology is well known to those skilled in the art who will readily appreciate that such probes may vary greatly in length and may be labeled with a detectable label, such as a radioisotope or flourescent dye, to facilitate detection of the probe. DNA probe molecules may be produced by insertion of a DNA molecule which encodes the polypeptides of this invention into suitable vectors, such as plasmids or bacteriophages, followed by transforming into suitable bacterial host cells, replication in the transformed bacterial host cells and harvesting of the DNA probes, using methods well known in the art. Alternatively, probes may be generated chemically from DNA synthesizers.

RNA probes may be generated by inserting the DNA molecule which encodes the polypeptides of this invention downstream of a bacteriophage promoter such as T3, T7, or SP6. Large amounts of RNA probe may be produced by incubating the labeled nucleotides with the linearized fragment where it contains an upstream promoter in the presence of the appropriate RNA polymerase.

This invention provides an antisense oligonucleotide having a sequence capable of specifically hybridizing to RNA encoding a human MCH1 receptor, so as to prevent translation of the RNA. This invention also provides an antisense oligonucleotide having a sequence capable of specifically hybridizing to genomic DNA encoding a human MCH1 receptor. In one embodiment, the oligonucleotide comprises chemically modified nucleotides or nucleotide analogues.

This invention provides an antibody capable of binding to a human MCH1 receptor encoded by a nucleic acid encoding a human MCH1 receptor. This invention also provides an agent capable of competitively inhibiting the binding of the antibody to a human MCH1 receptor. In one embodiment, the antibody is a monoclonal antibody or antisera.

This invention provides a pharmaceutical composition comprising (a) an amount of the oligonucleotide capable of passing through a cell membrane and effective to reduce expression of a human MCH1 receptor and (b) a pharmaceutically acceptable carrier capable of passing through the cell membrane. In an embodiment, the oligonucleotide is coupled to a substance which inactivates mRNA. In a further embodiment, the substance which inactivates mRNA is a ribozyme. In another embodiment, the pharmaceutically acceptable carrier comprises a structure which binds to a human MCH1 receptor on a cell capable of being taken up by the cells after binding to the structure. In a further embodiment, the pharmaceutically acceptable carrier is capable of binding to a human MCH1 receptor which is specific for a selected cell type.

This invention provides a pharmaceutical composition which comprises an amount of an antibody effective to block binding of a ligand to a human MCH1 receptor and a pharmaceutically acceptable carrier.

As used herein, the phrase “pharmaceutically acceptable carrier” means any of the standard pharmaceutically acceptable carriers and is any pharmaceutical carrier known to those of ordinary skill in the art as useful in formulating pharmaceutical compositions. Examples include, but are not limited to, phosphate buffered saline, physiological saline, water, and emulsions, such as oil/water emulsions.

On Dec. 24, 1997 the Food and Drug Administration of the United States Department of Health and Human Services published a guidance entitled “Q3C Impurities: Residual Solvent”. The guidance recommends acceptable amounts of residual solvents in pharmaceuticals for the safety of the patient, and recommends the use of less toxic solvents in the manufacture of drug substances and dosage forms. Table 1 of the guidance lists “Class 1 Solvents”. The guidance then states that the use of Class 1 Solvents should be avoided in the production of drug substances, excipients, or drug products unless their use can be strongly justified in a risk-benefit assessment. The guidance further states that Class 2 Solvents should be limited in order to protect patients from potentially adverse effects. The guidance characterized the following solvents as Class 1 Solvents: benzene, carbon tetrachloride, 1,2-dichloroethane, 1,1-dichloroethene, and 1,1,1-trichloroethane. The guidance characterized the following solvents as Class 2 Solvents: acetonitrile, chlorobenzene, chloroform, cyclohexane, 1,2-dichloroethene, dichloromethane, 1,2-dimethoxyethane, N,N-dimethylacetamide, N,N-dimethylformamide, 1,4-dioxane, 2-ethoxyethanol, ethyleneglycol, formamide, hexane, methanol, 2-methoxyethanol, methylbutyl ketone, methylcyclohexane, N-methylpyrrolidone, nitromethane, pyridine, sulfolane, tetralin, toluene, 1,1,2-trichloroethene and xylene. As used in this invention the term “pharmaceutically acceptable carrier” shall not include Class 1 or Class 2 Solvents.

In an embodiment of the present invention, the pharmaceutical carrier may be a liquid and the pharmaceutical composition would be in the form of a solution. In another embodiment, the pharmaceutically acceptable carrier is a solid and the composition is in the form of a powder or tablet. In a further embodiment, the pharmaceutical carrier is a gel and the composition is in the form of a suppository or cream. In a further embodiment the compound may be formulated as a part of a pharmaceutically acceptable transdermal patch. In yet a further embodiment, the compound may be delivered to the subject by means of a spray or inhalant.

A solid carrier can include one or more substances which may also act as endogenous carriers (e.g. nutrient or micronutrient carriers), flavoring agents, lubricants, solubilizers, suspending agents, fillers, glidants, compression aids, binders or tablet-disintegrating agents; it can also be an encapsulating material. In powders, the carrier is a finely divided solid which is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, polyvinylpyrrolidine, low melting waxes and ion exchange resins.

Liquid carriers are used in preparing solutions, suspensions, emulsions, syrups, elixirs and pressurized compositions. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmoregulators. Suitable examples of liquid carriers for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also be an oily ester such as ethyl oleate or isopropyl myristate. Sterile liquid carriers are useful in sterile liquid form compositions for parenteral administration. The liquid carrier for pressurized compositions can be halogenated hydrocarbon or other pharmaceutically acceptable propellent.

Liquid pharmaceutical compositions which are sterile solutions or suspensions can be utilized by for example, intramuscular, intrathecal, epidural, intraperitoneal or subcutaneous injection. Sterile solutions can also be administered intravenously. The compounds may be prepared as a sterile solid composition which may be dissolved or suspended at the time of administration using sterile water, saline, or other appropriate sterile injectable medium. Carriers are intended to include necessary and inert binders, suspending agents, lubricants, flavorants, sweeteners, preservatives, dyes, and coatings.

The MCH1 antagonist can be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like.

The MCH1 antagonist can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

Optimal dosages to be administered may be determined by those skilled in the art, and will vary with the particular compound in use, the strength of the preparation, the mode of administration, and the advancement of the disease condition. Additional factors depending on the particular subject being treated will result in a need to adjust dosages, including subject age, weight, gender, diet, and time of administration.

This invention provides a transgenic, nonhuman mammal expressing DNA encoding a human MCH1 receptor. This invention also provides a transgenic, nonhuman mammal comprising a homologous recombination knockout of the native human MCH1 receptor. This invention further provides a transgenic, nonhuman mammal whose genome comprises antisense DNA complementary to the DNA encoding a human MCH1 receptor so placed within the genome as to be transcribed into antisense mRNA which is complementary to mRNA encoding the human MCH1 receptor and which hybridizes to mRNA encoding the human MCH1 receptor, thereby reducing its translation. In an embodiment, the DNA encoding the human MCH1 receptor additionally comprises an inducible promoter. In another embodiment, the DNA encoding the human MCH1 receptor additionally comprises tissue specific regulatory elements. In a further embodiment, the transgenic, nonhuman mammal is a mouse.

Animal model systems which elucidate the physiological and behavioral roles of the polypeptides of this invention are produced by creating transgenic animals in which the activity of the polypeptide is either increased or decreased, or the amino acid sequence of the expressed polypeptide is altered, by a variety of techniques. Examples of these techniques include, but are not limited to: 1) Insertion of normal or mutant versions of DNA encoding the polypeptide, by microinjection, electroporation, retroviral transfection or other means well known to those in the art, into appropriate fertilized embryos in order to produce a transgenic animal or 2) Homologous recombination of mutant or normal, human or animal versions of these genes with the native gene locus in transgenic animals to alter the regulation of expression or the structure of these polypeptide sequences. The technique of homologous recombination is well known in the art. It replaces the native gene with the inserted gene and so is useful for producing an animal that cannot express native polypeptides but does express, for example, an inserted mutant polypeptide, which has replaced the native polypeptide in the animal's genome by recombination, resulting in underexpression of the transporter. Microinjection adds genes to the genome, but does not remove them, and so is useful for producing an animal which expresses its own and added polypeptides, resulting in overexpression of the polypeptides.

One means available for producing a transgenic animal, with a mouse as an example, is as follows: Female mice are mated, and the resulting fertilized eggs are dissected out of their oviducts. The eggs are stored in an appropriate medium such as M2 medium. DNA or cDNA encoding a polypeptide of this invention is purified from a vector by methods well known in the art. Inducible promoters may be fused with the coding region of the DNA to provide an experimental means to regulate expression of the trans-gene. Alternatively, or in addition, tissue specific regulatory elements may be fused with the coding region to permit tissue-specific expression of the trans-gene. The DNA, in an appropriately buffered solution, is put into a microinjection needle (which may be made from capillary tubing using a pipette puller) and the egg to be injected is put in a depression slide. The needle is inserted into the pronucleus of the egg, and the DNA solution is injected. The injected egg is then transferred into the oviduct of a pseudopregnant mouse (a mouse stimulated by the appropriate hormones to maintain pregnancy but which is not actually pregnant ), where it proceeds to the uterus, implants, and develops to term. As noted above, microinjection is not the only method for inserting DNA into the egg cell, and is used here only for exemplary purposes.

This invention provides a process for identifying a chemical compound which specifically binds to a mammalian MCH1 receptor which comprises contacting cells comprising DNA encoding, and expressing on their cell surface, the mammalian MCH1 receptor, with the compound under conditions suitable for binding, and detecting specific binding of the chemical compound to the mammalian MCH1 receptor, wherein the cells do not normally express the mammalian MCH1 receptor and the DNA encoding the mammalian MCH1 receptor (a) hybridizes to a nucleic acid having the defined sequence shown in FIG. 1 (SEQ ID NO: 1) under low stringency conditions or a sequence complementary thereto and (b) is further characterized by its ability to cause a change in the pH of a culture of CHO cells when a MCH1 ligand is added to the culture and the CHO cells contain the nucleic acid which hybridized to the nucleic acid having the defined sequence or its complement. This invention also provides a process for identifying a chemical compound which specifically binds to a mammalian MCH1 receptor which comprises contacting a membrane preparation from cells comprising DNA encoding, and expressing on their cell surface, the mammalian MCH1 receptor, with the compound under conditions suitable for binding, and detecting specific binding of the chemical compound to the mammalian MCH1 receptor, wherein the cells do not normally express the mammalian MCH1 receptor and the DNA encoding the mammalian MCH1 receptor (a) hybridizes to a nucleic acid having the defined sequence shown in FIG. 1 (SEQ ID NO: 1) under low stringency conditions or a sequence complementary thereto and (b) is further characterized by its ability to cause a change in the pH of a culture of CHO cells when a MCH1 ligand is added to the culture and the CHO cells contain the nucleic acid which hybridized to the nucleic acid having the defined sequence or its complement. In one embodiment, the MCH1 receptor is a human MCH1 receptor. In another embodiment, the MCH1 receptor is a rat MCH1 receptor. In another embodiment, the mammalian MCH1 receptor comprises substantially the same amino acid sequence as the sequence of the human MCH1 receptor encoded by plasmid pEXJ.HR-TL231. In a further embodiment, the mammalian MCH1 receptor comprises substantially the same amino acid sequence as that shown in FIG. 2 (SEQ ID NO: 2). In another embodiment, the mammalian MCH1 receptor comprises the amino acid sequence shown in FIG. 2 (SEQ ID NO: 2). In a different embodiment, the mammalian MCH1 receptor comprises the amino acid sequence shown in FIG. 13 (SEQ ID NO: 26). In another embodiment, the mammalian MCH1 receptor comprises the amino acid sequence shown in FIG. 14 (SEQ ID NO: 27). In still another embodiment, the mammalian MCH1 receptor comprises the amino acid sequence shown in FIG. 15 (SEQ ID NO: 28). In one embodiment, the compound is not previously known to bind to a mammalian MCH1 receptor. This invention further provides a compound identified by the above-described processes.

In one embodiment of the above-described processes, the cell is an insect cell. In another embodiment, the cell is a mammalian cell. In a further embodiment, the cell is nonneuronal in origin. In a further embodiment, the nonneuronal cell is a COS-7 cell, 293 human embryonic kidney cell, a CHO cell, a NIH-3T3 cell, a mouse Y1 cell, or a LM(tk−) cell.

This invention provides a process involving competitive binding for identifying a chemical compound which specifically binds to a mammalian MCH1 receptor which comprises contacting cells expressing on their cell surface the mammalian MCH1 receptor, with both the chemical compound and a second chemical compound known to bind to the receptor, and separately with only the second chemical compound, under conditions suitable for binding of both compounds, and detecting specific binding of the chemical compound to the mammalian MCH1 receptor, a decrease in the binding of the second chemical compound to the mammalian MCH1 receptor in the presence of the chemical compound indicating that the chemical compound binds to the mammalian MCH1 receptor, wherein the cells do not normally express the mammalian MCH1 receptor and the DNA encoding the mammalian MCH1 receptor (a) hybridizes to a nucleic acid having the defined sequence shown in FIG. 1 (SEQ ID NO: 1) under low stringency conditions or a sequence complementary thereto and (b) is further characterized by its ability to cause a change in the pH of a culture of CHO cells when a MCH1 ligand is added to the culture and the CHO cells contain the nucleic acid which hybridized to the nucleic acid having the defined sequence or its complement.

This invention also provides a process involving competitive binding for identifying a chemical compound which specifically binds to a mammalian MCH1 receptor which comprises contacting a membrane preparation from cells expressing on their cell surface the mammalian MCH1 receptor, with both the chemical compound and a second chemical compound known to bind to the receptor, and separately with only the second chemical compound, under conditions suitable for binding of both compounds, and detecting specific binding of the chemical compound to the mammalian MCH1 receptor, a decrease in the binding of the second chemical compound to the mammalian MCH1 receptor in the presence of the chemical compound indicating that the chemical compound binds to the mammalian MCH1 receptor, wherein the cells do not normally express the mammalian MCH1 receptor and the DNA encoding the mammalian MCH1 receptor (a) hybridizes to a nucleic acid having the defined sequence shown in FIG. 1 (SEQ ID NO: 1) under low stringency conditions or a sequence complementary thereto and (b) is further characterized by its ability to cause a change in the pH of a culture of CHO cells when a MCH1 ligand is added to the culture and the CHO cells contain the nucleic acid which hybridized to the nucleic acid having the defined sequence or its complement.

In one embodiment, the mammalian MCH1 receptor is a human MCH1 receptor or a mutant of such human MCH1 receptor which is activated by MCH or an analog or homolog thereof. In another embodiment, the mammalian MCH1 receptor is a rat MCH1 receptor. In another embodiment, the mammalian MCH1 receptor comprises substantially the same amino acid sequence as the human MCH1 receptor encoded by plasmid pEXJ.HR-TL231. In a further embodiment, the mammalian MCH1 receptor comprises substantially the same amino acid sequence as that shown in FIG. 2 (SEQ ID NO: 2). In another embodiment, the mammalian MCH1 receptor comprises the amino acid sequence shown in FIG. 2 (SEQ ID NO: 2).

In one embodiment, the cell is an insect cell. In another embodiment, the cell is a mammalian cell. In a further embodiment, the cell is nonneuronal in origin. In another embodiment, the nonneuronal cell is a COS-7 cell, 293 human embryonic kidney cell, a CHO cell, a NIH-3T3 cell, a mouse Y1 cell, or a LM(tk−) cell. In one embodiment, the compound is not previously known to bind to a mammalian MCH1 receptor.

This invention provides a compound identified by the above-described processes.

This invention provides a method of screening a plurality of chemical compounds not known to bind to a mammalian MCH1 receptor to identify a compound which specifically binds to the mammalian MCH1 receptor, which comprises (a) contacting cells transfected with and expressing DNA encoding the mammalian MCH1 receptor with the plurality of compounds not known to bind specifically to the mammalian MCH1 receptor, under conditions permitting binding of compounds known to bind the mammalian MCH1 receptor; (b) determining whether the binding of a compound known to bind to the mammalian MCH1 receptor is reduced in the presence of the compounds within the plurality of compounds, relative to the binding of the compound in the absence of the plurality of compounds; and if so (c) separately determining the binding to the mammalian MCH1 receptor of compounds included in the plurality of compounds, so as to thereby identify the compound which specifically binds to the mammalian MCH1 receptor.

This invention provides a method of screening a plurality of chemical compounds not known to bind to a mammalian MCH1 receptor to identify a compound which specifically binds to the mammalian MCH1 receptor, which comprises (a) contacting a membrane preparation from cells transfected with and expressing the mammalian MCH1 receptor with the plurality of compounds not known to bind specifically to the mammalian MCH1 receptor, under conditions permitting binding of compounds known to bind the mammalian MCH1 receptor; (b) determining whether the binding of a compound known to bind to the mammalian MCH1 receptor is reduced in the presence of the compounds within the plurality of compounds, relative to the binding of the compound in the absence of the plurality of compounds; and if so (c) separately determining the binding to the mammalian MCH1 receptor of compounds included in the plurality of compounds, so as to thereby identify the compound which specifically binds to the mammalian MCH1 receptor.

In one embodiment of the above-described methods, the mammalian MCH1 receptor is a human MCH1 receptor or a mutant of such human MCH1 receptor which is activated by MCH or an analog or homolog thereof. In another embodiment, the mammalian MCH1 receptor is a rat MCH1 receptor. In another embodiment, the cell is a mammalian cell. In a further embodiment, the mammalian cell is non-neuronal in origin. In another embodiment, the non-neuronal cell is a COS-7 cell, a 293 human embryonic kidney cell, a LM(tk−) cell, a CHO cell, a mouse Y1 cell, or an NIH-3T3 cell.

This invention also provides a method of detecting expression of a mammalian MCH1 receptor by detecting the presence of mRNA coding for the mammalian MCH1 receptor which comprises obtaining total mRNA from the cell and contacting the mRNA so obtained from a nucleic acid probe under hybridizing conditions, detecting the presence of mRNA hybridizing to the probe, and thereby detecting the expression of the mammalian MCH1 receptor by the cell.

This invention further provides a method of detecting the presence of a mammalian MCH1 receptor on the surface of a cell which comprises contacting the cell with an antibody under conditions permitting binding of the antibody to the receptor, detecting the presence of the antibody bound to the cell, and thereby detecting the presence of the mammalian MCH1 receptor on the surface of the cell.

This invention provides a method of determining the physiological effects of varying levels of activity of human MCH1 receptors which comprises producing a transgenic, nonhuman mammal whose levels of human MCH1 receptor activity are varied by use of an inducible promoter which regulates human MCH1 receptor expression.

This invention also provides a method of determining the physiological effects of varying levels of activity of human MCH1 receptors which comprises producing a panel of transgenic, nonhuman mammals each expressing a different amount of human MCH1 receptor.

This invention provides a method for identifying an antagonist capable of alleviating an abnormality wherein the abnormality is alleviated by decreasing the activity of a human MCH1 receptor comprising administering a compound to a transgenic, nonhuman mammal, and determining whether the compound alleviates the physical and behavioral abnormalities displayed by the transgenic, nonhuman mammal as a result of overactivity of a human MCH1 receptor, the alleviation of the abnormality identifying the compound as an antagonist. This invention also provides an antagonist identified by the above-described method. This invention further provides a pharmaceutical composition comprising an antagonist identified by the above-described method and a pharmaceutically acceptable carrier. This invention provides a method of treating an abnormality in a subject wherein the abnormality is alleviated by decreasing the activity of a human MCH1 receptor which comprises administering to the subject an effective amount of this pharmaceutical composition, thereby treating the abnormality.

This invention provides a method for identifying an agonist capable of alleviating an abnormality in a subject wherein the abnormality is alleviated by increasing the activity of a human MCH1 receptor comprising administering a compound to transgenic, nonhuman mammal, and determining whether the compound alleviates the physical and behavioral abnormalities displayed by the transgenic, nonhuman mammal, the alleviation of the abnormality identifying the compound as an agonist. This invention also provides an agonist identified by the above-described method. This invention further provides a pharmaceutical composition comprising an agonist identified by the above-described method and a pharmaceutically acceptable carrier. This invention further provides a method of treating an abnormality in a subject wherein the abnormality is alleviated by increasing the activity of a human MCH1 receptor which comprises administering to the subject an effective amount of this pharmaceutical composition, thereby treating the abnormality.

This invention provides a method for diagnosing a predisposition to a disorder associated with the activity of a specific mammalian allele which comprises: (a) obtaining DNA of subjects suffering from the disorder; (b) performing a restriction digest of the DNA with a panel of restriction enzymes; (c) electrophoretically separating the resulting DNA fragments on a sizing gel; (d) contacting the resulting gel with a nucleic acid probe capable of specifically hybridizing with a unique sequence included within the sequence of a nucleic acid molecule encoding a human MCH1 receptor and labeled with a detectable marker; (e) detecting labeled bands which have hybridized to the DNA encoding a human MCH1 receptor labeled with a detectable marker to create a unique band pattern specific to the DNA of subjects suffering from the disorder; (f) preparing DNA obtained for diagnosis by steps (a)-(e); and (g) comparing the unique band pattern specific to the DNA of subjects suffering from the disorder from step (e) and the DNA obtained for diagnosis from step (f) to determine whether the patterns are the same or different and to diagnose thereby predisposition to the disorder if the patterns are the same. In one embodiment, a disorder associated with the activity of a specific mammalian allele is diagnosed.

This invention provides a method of preparing the purified human MCH1 receptor which comprises: (a) inducing cells to express the human MCH1 receptor; (b) recovering the human MCH1 receptor from the induced cells; and (c) purifying the human MCH1 receptor so recovered.

This invention provides a method of preparing the purified human MCH1 receptor which comprises: (a) inserting nucleic acid encoding the human MCH1 receptor in a suitable vector; (b) introducing the resulting vector in a suitable host cell; (c) placing the resulting cell in suitable condition permitting the production of the isolated human MCH1 receptor; (d) recovering the human MCH1 receptor produced by the resulting cell; and (e) purifying the human MCH1 receptor so recovered.

This invention provides a process for determining whether a chemical compound is a mammalian MCH1 receptor agonist which comprises contacting cells transfected with and expressing DNA encoding the mammalian MCH1 receptor with the compound under conditions permitting the activation of the mammalian MCH1 receptor, and detecting an increase in mammalian MCH1 receptor activity, so as to thereby determine whether the compound is a mammalian MCH1 receptor agonist. This invention also provides a process for determining whether a chemical compound is a mammalian MCH1 receptor antagonist which comprises contacting cells transfected with and expressing DNA encoding the mammalian MCH1 receptor with the compound in the presence of a known mammalian MCH1 receptor agonist, under conditions permitting the activation of the mammalian MCH1 receptor, and detecting a decrease in mammalian MCH1 receptor activity, so as to thereby determine whether the compound is a mammalian MCH1 receptor antagonist. In one embodiment, the mammalian MCH1 receptor is a human MCH1 receptor or a mutant of such human MCH1 receptor which is activated by MCH or an analog or homolog thereof.

This invention further provides a pharmaceutical composition which comprises an amount of a mammalian MCH1 receptor agonist determined by the above-described process effective to increase activity of a mammalian MCH1 receptor and a pharmaceutically acceptable carrier. In one embodiment, the mammalian MCH1 receptor agonist is not previously known.

This invention provides a pharmaceutical composition which comprises an amount of a mammalian MCH1 receptor antagonist determined by the above-described process effective to reduce activity of a mammalian MCH1 receptor and a pharmaceutically acceptable carrier. In one embodiment, the mammalian MCH1 receptor antagonist is not previously known.

This invention provides a process for determining whether a chemical compound specifically binds to and activates a mammalian MCH1 receptor, which comprises contacting cells producing a second messenger response and expressing on their cell surface the mammalian MCH1 receptor, wherein such cells do not normally express the mammalian MCH1 receptor, with the chemical compound under conditions suitable for activation of the mammalian MCH1 receptor, and measuring the second messenger response in the presence and in the absence of the chemical compound, a change in the second messenger response in the presence of the chemical compound indicating that the compound activates the mammalian MCH1 receptor. In one embodiment, the second messenger response comprises chloride channel activation and the change in second messenger is an increase in the level of inward chloride current.

This invention also provides a process for determining whether a chemical compound specifically binds to and inhibits activation of a mammalian MCH1 receptor, which comprises separately contacting cells producing a second messenger response and expressing on their cell surface the mammalian MCH1 receptor, wherein such cells do not normally express the mammalian MCH1 receptor, with both the chemical compound and a second chemical compound known to activate the mammalian MCH1 receptor, and with only the second chemical compound, under conditions suitable for activation of the mammalian MCH1 receptor, and measuring the second messenger response in the presence of only the second chemical compound and in the presence of both the second chemical compound and the chemical compound, a smaller change in the second messenger response in the presence of both the chemical compound and the second chemical compound than in the presence of only the second chemical compound indicating that the chemical compound inhibits activation of the mammalian MCH1 receptor. In one embodiment, the second messenger response comprises chloride channel activation and the change in second messenger response is a smaller increase in the level of inward chloride current in the presence of both the chemical compound and the second chemical compound than in the presence of only the second chemical compound. This invention also provides the above-described processes performed with membrane preparations from cells producing a second messenger response and transfected with and expressing the mammalian MCH1 receptor.

In one embodiment of the above-described processes, the mammalian MCH1 receptor is a human MCH1 receptor or a mutant of such human MCH1 receptor which is activated by MCH or an analog or homolog thereof. In another embodiment, the mammalian MCH1 receptor is a rat MCH1 receptor. In another embodiment, the mammalian MCH1 receptor comprises substantially the same amino acid sequence as encoded by the plasmid pEXJ.HR-TL231. In a further embodiment, the mammalian MCH1 receptor comprises substantially the same amino acid sequence as that shown in FIG. 2 (SEQ ID NO: 2). In another embodiment, the mammalian MCH1 receptor comprises an amino acid sequence as shown in FIG. 2 (SEQ ID NO: 2). In an embodiment, the cell is an insect cell. In a further embodiment, the cell is a mammalian cell. In a still further embodiment, the mammalian cell is nonneuronal in origin. In another embodiment, the nonneuronal cell is a COS-7 cell, CHO cell, 293 human embryonic kidney cell, NIH-3T3 cell or LM(tk−) cell. In an embodiment, the compound is not previously known to bind to a mammalian MCH1 receptor. This invention also provides a compound determined by the above-described processes.

This invention also provides a pharmaceutical composition which comprises an amount of a mammalian MCH1 receptor agonist determined by the above-described processes effective to increase activity of a mammalian MCH1 receptor and a pharmaceutically acceptable carrier. In one embodiment, the mammalian MCH1 receptor agonist is not previously known.

This invention further provides a pharmaceutical composition which comprises an amount of a mammalian MCH1 receptor antagonist determined by the above-described processes effective to reduce activity of a mammalian MCH1 receptor and a pharmaceutically acceptable carrier. In one embodiment, the mammalian MCH1 receptor antagonist is not previously known.

This invention provides a method of screening a plurality of chemical compounds not known to activate a mammalian MCH1 receptor to identify a compound which activates the mammalian MCH1 receptor which comprises: (a) contacting cells transfected with and expressing the mammalian MCH1 receptor with the plurality of compounds not known to activate the mammalian MCH1 receptor, under conditions permitting activation of the mammalian MCH1 receptor; (b) determining whether the activity of the mammalian MCH1 receptor is increased in the presence of the compounds; and if so (c) separately determining whether the activation of the mammalian MCH1 receptor is increased by each compound included in the plurality of compounds, so as to thereby identify the compound which activates the mammalian MCH1 receptor. In one embodiment, the mammalian MCH1 receptor is a human MCH1 receptor or a mutant of such human MCH1 receptor which is activated by MCH or an analog or homolog thereof. In another embodiment, the mammalian MCH1 receptor is a rat MCH1 receptor.

This invention provides a method of screening a plurality of chemical compounds not known to inhibit the activation of a mammalian MCH1 receptor to identify a compound which inhibits the activation of the mammalian MCH1 receptor, which comprises: (a) contacting cells transfected with and expressing the mammalian MCH1 receptor with the plurality of compounds in the presence of a known mammalian MCH1 receptor agonist, under conditions permitting activation of the mammalian MCH1 receptor; (b) determining whether the activation of the mammalian MCH1 receptor is reduced in the presence of the plurality of compounds, relative to the activation of the mammalian MCH1 receptor in the absence of the plurality of compounds; and if so (c) separately determining the inhibition of activation of the mammalian MCH1 receptor for each compound included in the plurality of compounds, so as to thereby identify the compound which inhibits the activation of the mammalian MCH1 receptor. In one embodiment, the mammalian MCH1 receptor is a human MCH1 receptor or a mutant of such human MCH1 receptor which is activated by MCH or an analog or homolog thereof. In another embodiment, the mammalian MCH1 receptor is a rat MCH1 receptor.

In one embodiment of the above-described methods, the cell is a mammalian cell. In another embodiment, the mammalian cell is non-neuronal in origin. In a further embodiment, the non-neuronal cell is a COS-7 cell, a 293 human embryonic kidney cell, a LM(tk−) cell or an NIH-3T3 cell.

This invention provides a pharmaceutical composition comprising a compound identified by the above-described methods effective to increase mammalian MCH1 receptor activity and a pharmaceutically acceptable carrier.

This invention also provides a pharmaceutical composition comprising a compound identified by the above-described methods effective to decrease mammalian MCH1 receptor activity and a pharmaceutically acceptable carrier.

This invention further provides a method of measuring receptor activation in an oocyte expression system such as a Xenopus oocyte expression system or melanophore. In an embodiment, receptor activation is determined by measurement of ion channel activity. In another embodiment, receptor activation is measured by aequorin luminescence.

Expression of genes in Xenopus oocytes is well known in the art (Coleman, A., 1984; Masu, Y.,et al., 1994) and is performed using microinjection of native mRNA or in vitro synthesized mRNA into frog oocytes. The preparation of in vitro synthesized mRNA can be performed by various standard techniques (Sambrook, et al. 1989) including using T7 polymerase with the mCAP RNA mapping kit (Stratagene).

This invention provides a method of treating an abnormality in a subject wherein the abnormality is alleviated by increasing the activity of a mammalian MCH1 receptor which comprises administering to the subject an amount of a compound which is a mammalian MCH1 receptor agonist effective to treat the abnormality. In separate embodiments, the abnormality is a regulation of a steroid or pituitary hormone disorder, an epinephrine release disorder, a gastrointestinal disorder, a cardiovascular disorder, an electrolyte balance disorder, hypertension, diabetes, a respiratory disorder, asthma, a reproductive function disorder, an immune disorder, an endocrine disorder, a musculoskeletal disorder, a neuroendocrine disorder, a cognitive disorder, a memory disorder such as Alzheimer's disease, a sensory modulation and transmission disorder, a motor coordination disorder, a sensory integration disorder, a motor integration disorder, a dopaminergic function disorder such as Parkinson's disease, a sensory transmission disorder, an olfaction disorder, a sympathetic innervation disorder, an affective disorder such as depression, a stress-related disorder, a fluid-balance disorder, a urinary disorder such as urinary incontinence, a seizure disorder, pain, psychotic behavior such as schizophrenia, morphine tolerance, opiate addiction or migraine.

This invention provides a method of treating an abnormality in a subject wherein the abnormality is alleviated by decreasing the activity of a mammalian MCH1 receptor which comprises administering to the subject an amount of a compound which is a mammalian MCH1 receptor antagonist effective to treat the abnormality. In separate embodiments, the abnormality is a regulation of a steroid or pituitary hormone disorder, an epinephrine release disorder, a gastrointestinal disorder, a cardiovascular disorder, an electrolyte balance disorder, hypertension, diabetes, a respiratory disorder, asthma, a reproductive function disorder, an immune disorder, an endocrine disorder, a musculoskeletal disorder, a neuroendocrine disorder, a cognitive disorder, a memory disorder such as Alzheimer's disease, a sensory modulation and transmission disorder, a motor coordination disorder, a sensory integration disorder, a motor integration disorder, a dopaminergic function disorder such as Parkinson's disease, a sensory transmission disorder, an olfaction disorder, a sympathetic innervation disorder, an affective disorder such as depression, a stress-related disorder, a fluid-balance disorder, a urinary disorder such as urinary incontinence, a seizure disorder, pain, psychotic behavior such as schizophrenia, morphine tolerance, opiate addiction or migraine.

This invention provides a process for making a composition of matter which specifically binds to a mammalian MCH1 receptor which comprises identifying a chemical compound using any of the processes described herein for identifying a compound which binds to and/or activates or inhibits activation of a mammalian MCH1 receptor and then synthesizing the chemical compound or a novel structural and functional analog or homolog thereof. In one embodiment, the mammalian MCH1 receptor is a human MCH1 receptor or a mutant of such human MCH1 receptor which is activated by MCH or an analog or homolog thereof. In another embodiment, the mammalian MCH1 receptor is a rat MCH1 receptor.

This invention further provides a process for preparing a composition which comprises admixing a pharmaceutically acceptable carrier and a therapeutically effective amount of a chemical compound identified by any of the processes described herein for identifying a compound which binds to and/or activates or inhibits activation of a mammalian MCH1 receptor or a novel structural and functional analog or homolog thereof. In one embodiment, the mammalian MCH1 receptor is a human MCH1 receptor or a mutant of such human MCH1 receptor which is activated by MCH or an analog or homolog thereof. In another embodiment, the mammalian MCH1 receptor is a rat MCH1 receptor.

This invention provides a process for determining whether a chemical compound is a human MCH1 receptor antagonist which comprises contacting cells transfected with and expressing DNA encoding the human MCH1 receptor with the compound in the presence of a known human MCH1 receptor agonist, under conditions permitting the activation of the human MCH1 receptor, and detecting a decrease in human MCH1 receptor activity, so as to thereby determine whether the compound is a human MCH1 receptor antagonist, wherein the DNA encoding the human MCH1 receptor comprises the sequence shown in FIG. 1 (Seq. ID No. 1) or contained in plasmid pEXJ.HR-TL231 (ATCC Accession No. 203197), the known human MCH1 receptor agonist is MCH or a homolog or analog of MCH, and the cells do not express the MCH1 receptor prior to transfecting them.

This invention also provides a process for determining whether a chemical compound specifically binds to and inhibits activation of a human MCH1 receptor, which comprises separately contacting cells expressing on their cell surface the human MCH1 receptor and producing a second messenger response upon activation of the human MCH1 receptor, wherein such cells do not normally express the human MCH1 receptor and the DNA encoding the human MCH1 receptor comprises the sequence shown in FIG. 1 (Seq. ID No. 1) or contained in plasmid pEXJ.HR-TL231 (ATCC Accession No. 203197), with both the chemical compound and a second chemical compound known to activate the human MCH1 receptor, and with only the second chemical compound, under conditions suitable for activation of the human MCH1 receptor, and measuring the second messenger response in the presence of only the second chemical compound and in the presence of both the second chemical compound and the chemical compound, a smaller change in the second messenger response in the presence of both the chemical compound and the second chemical compound than in the presence of only the second chemical compound indicating that the chemical compound inhibits activation of the human MCH1 receptor, wherein the second chemical compound is MCH or a homolog or analog of MCH. In one embodiment, the second messenger response comprises chloride channel activation and the change in second messenger response is a smaller increase in the level of inward chloride current in the presence of both the chemical compound and the second chemical compound than in the presence of only the second chemical compound. This invention further provides a method of screening a plurality of chemical compounds not known to inhibit the activation of a human MCH1 receptor to identify a compound which inhibits the activation of the human MCH1 receptor, which comprises:

(a) contacting cells transfected with and expressing the human MCH1 receptor, wherein such cells do not normally express the human MCH1 receptor and the DNA encoding the human MCH1 receptor comprises the sequence shown in FIG. 1 (Seq. ID No. 1) or contained in plasmid pEXJ.HR-TL231 (ATCC Accession No. 203197), with the plurality of compounds in the presence of a known human MCH1 receptor agonist, under conditions permitting activation of the human MCH1 receptor, wherein the known MCH1 receptor agonist is MCH or a homolog or analog of MCH;

(b) determining whether the activation of the human MCH1 receptor is reduced in the presence of the plurality of compounds, relative to the activation of the human MCH1 receptor in the absence of the plurality of compounds; and if so

(c) separately determining the extent of inhibition of activation of the human MCH1 receptor for each compound included in the plurality of compounds, so as to thereby identify the compound which inhibits the activation of the human MCH1 receptor.

In one embodiment of the above-described methods, the cell is an insect cell. In another embodiment, the cell is a mammalian cell. In still another embodiment, the cell is a mammalian cell which is nonneuronal in origin. In further embodiments, the cell is a COS-7 cell, a CHO cell, a 293 human embryonic kidney cell, a NIH-3T3 cell, a mouse Y1 cell, or a LM(tk−) cell.

This invention provides a process for making a composition of matter which specifically binds to a human MCH1 receptor which comprises identifying a chemical compound which specifically binds to the human MCH1 receptor and then synthesizing the chemical compound or a structural and functional analog or homolog thereof, wherein the chemical compound is identified as binding to the human MCH1 receptor by a process involving competitive binding which comprises contacting cells expressing on their cell surface the human MCH1 receptor, with both the chemical compound and a second chemical compound known to bind to the receptor, and separately with only the second chemical compound, under conditions suitable for binding of both compounds, and detecting the extent of specific binding of the chemical compound to the human MCH1 receptor, a decrease in the binding of the second chemical compound to the human MCH1 receptor in the presence of the chemical compound indicating that the chemical compound binds to the human MCH1 receptor, wherein the cells do not normally express the human MCH1 receptor, the human MCH1 receptor is encoded by nucleic acid comprising the sequence shown in FIG. 1 (Seq. ID No. 1) or contained in plasmid pEXJ.HR-TL231 (ATCC Accession No. 203197), and the second chemical compound is MCH or a homolog or analog of MCH.

This invention further provides a process for making a composition of matter which specifically binds to a human MCH1 receptor which comprises identifying a chemical compound which specifically binds to the human MCH1 receptor and then synthesizing the chemical compound or a structural and functional analog or homolog thereof, wherein the chemical compound is identified as binding to the human MCH1 receptor by a process involving competitive binding which comprises contacting a membrane preparation from cells expressing on their cell surface the human MCH1 receptor, with both the chemical compound and a second chemical compound known to bind to the receptor, and separately with only the second chemical compound, under conditions suitable for binding of both compounds, and detecting the extent of specific binding of the chemical compound to the human MCH1 receptor, a decrease in the binding of the second chemical compound to the human MCH1 receptor in the presence of the chemical compound indicating that the chemical compound binds to the human MCH1 receptor, wherein the cells do not normally express the human MCH1 receptor, the human MCH1 receptor is encoded by nucleic acid comprising the sequence shown in FIG. 1 (Seq. ID No. 1) or contained in plasmid pEXJ.HR-TL231 (ATCC Accession No. 203197), and the second chemical compound is MCH or a homolog or analog of MCH.

This invention also provides a process for making a composition of matter which is a human MCH1 receptor antagonist which comprises identifying a chemical compound which is a human MCH1 receptor antagonist and then synthesizing the chemical compound or a structural and functional analog or homolog thereof, wherein the chemical compound is identified as a human MCH1 receptor antagonist by a process which comprises contacting cells transfected with and expressing DNA encoding the human MCH1 receptor with the compound in the presence of a known human MCH1 receptor agonist, under conditions permitting the activation of the human MCH1 receptor, and detecting a decrease in human MCH1 receptor activity, so as to thereby determine whether the compound is a human MCH1 receptor antagonist, wherein the cells do not normally express the human MCH1 receptor, the human MCH1 receptor is encoded by nucleic acid comprising the sequence shown in FIG. 1 (Seq. ID No. 1) or contained in plasmid pEXJ.HR-TL231 (ATCC Accession No. 203197), and the known human MCH1 receptor agonist is MCH or a homolog or analog of MCH.

This invention still further provides a process for making a composition of matter which specifically binds to and inhibits the activation of a human MCH1 receptor which comprises identifying a chemical compound which specifically binds to and inhibits the activation of the human MCH1 receptor and then synthesizing the chemical compound or a structural and functional analog or homolog thereof, wherein the chemical compound is identified as binding to and inhibiting the activation of the human MCH1 receptor by a process which comprises separately contacting cells expressing on their cell surface the human MCH1 receptor and producing a second messenger response upon activation of the human MCH1 receptor, wherein such cells do not normally express the human MCH1 receptor and the human MCH1 receptor is encoded by nucleic acid comprising the sequence shown in FIG. 1 (Seq. ID No. 1) or contained in plasmid pEXJ.HR-TL231 (ATCC Accession No. 203197), with both the chemical compound and a second chemical compound known to activate the human MCH1 receptor, and with only the second chemical compound, under conditions suitable for activation of the human MCH1 receptor, and measuring the second messenger response in the presence of only the second chemical compound and in the presence of both the second chemical compound and the chemical compound, a smaller change in the second messenger response in the presence of both the chemical compound and the second chemical compound than in the presence of only the second chemical compound indicating that the chemical compound inhibits activation of the human MCH1 receptor, wherein the second chemical compound is MCH or a homolog or analog of MCH. In one embodiment, the second messenger response comprises chloride channel activation and the change in second messenger response is a smaller increase in the level of inward chloride current in the presence of both the chemical compound and the second chemical compound than in the presence of only the second chemical compound.

This invention provides a process for preparing a composition which comprises identifying a chemical compound which specifically binds to a human MCH1 receptor, and then admixing a carrier and the chemical compound or a structural and functional analog or homolog thereof, wherein the chemical compound is identified as binding to the human MCH1 receptor by a process involving competitive binding which comprises contacting cells expressing on their cell surface the human MCH1 receptor, with both the chemical compound and a second chemical compound known to bind to the receptor, and separately with only the second chemical compound, under conditions suitable for binding of both compounds, and detecting the extent of specific binding of the chemical compound to the human MCH1 receptor, a decrease in the binding of the second chemical compound to the human MCH1 receptor in the presence of the chemical compound indicating that the chemical compound binds to the human MCH1 receptor, wherein the cells do not normally express the human MCH1 receptor, the human MCH1 receptor is encoded by nucleic acid comprising the sequence shown in FIG. 1 (Seq. ID No. 1) or contained in plasmid pEXJ.HR-TL231 (ATCC Accession No. 203197), and the second chemical compound is MCH or a homolog or analog of MCH.

This invention further provides a process for preparing a composition which comprises identifying a chemical compound which specifically binds to a human MCH1 receptor, and then admixing a carrier and the chemical compound or a structural and functional analog or homolog thereof, wherein the chemical compound is identified as binding to the human MCH1 receptor by a process involving competitive binding which comprises contacting a membrane preparation from cells expressing on their cell surface the human MCH1 receptor, with both the chemical compound and a second chemical compound known to bind to the receptor, and separately with only the second chemical compound, under conditions suitable for binding of both compounds, and detecting the extent of specific binding of the chemical compound to the human MCH1 receptor, a decrease in the binding of the second chemical compound to the human MCH1 receptor in the presence of the chemical compound indicating that the chemical compound binds to the human MCH1 receptor, wherein the cells do not normally express the human MCH1 receptor, the human MCH1 receptor is encoded by nucleic acid comprising the sequence shown in FIG. 1 (Seq. ID No. 1) or contained in plasmid pEXJ.HR-TL231 (ATCC Accession No. 203197), and the second chemical compound is MCH or a homolog or analog of MCH.

This invention also provides a process for preparing a composition which comprises identifying a chemical compound which is a human MCH1 receptor antagonist, and then admixing a carrier and the chemical compound or a structural and functional analog or homolog thereof, wherein the chemical compound is identified as a human MCH1 receptor antagonist by a process which comprises contacting cells transfected with and expressing DNA encoding the human MCH1 receptor with the compound in the presence of a known human MCH1 receptor agonist, under conditions permitting the activation of the human MCH1 receptor, and detecting a decrease in human MCH1 receptor activity, so as to thereby determine whether the compound is a human MCH1 receptor antagonist, wherein the cells do not normally express the human MCH1 receptor, the human MCH1 receptor is encoded by nucleic acid comprising the sequence shown in FIG. 1 (Seq. ID No. 1) or contained in plasmid pEXJ.HR-TL231 (ATCC Accession No. 203197), and the known human MCH1 receptor agonist is MCH or a homolog or analog of MCH.

This invention still further provides a process for preparing a composition which comprises identifying a chemical compound which specifically binds to and inhibits the activation of a human MCH1 receptor, and then admixing a carrier and the chemical compound or a structural and functional analog or homolog thereof, wherein the chemical compound is identified as binding to and inhibiting activation of the human MCH1 receptor by a process which comprises separately contacting cells expressing on their cell surface the human MCH1 receptor and producing a second messenger response upon activation of the human MCH1 receptor, wherein such cells do not normally express the human MCH1 receptor and the human MCH1 receptor is encoded by nucleic acid comprising the sequence shown in FIG. 1 (Seq. ID No. 1) or contained in plasmid PEXJ.HR-TL231 (ATCC Accession No. 203197), with both the chemical compound and a second chemical compound known to activate the human MCH1 receptor, and with only the second chemical compound, under conditions suitable for activation of the human MCH1 receptor, and measuring the second messenger response in the presence of only the second chemical compound and in the presence of both the second chemical compound and the chemical compound, a smaller change in the second messenger response in the presence of both the chemical compound and the second chemical compound than in the presence of only the second chemical compound indicating that the chemical compound inhibits activation of the human MCH1 receptor, wherein the second chemical compound is MCH or a homolog or analog of MCH. In one embodiment, the second messenger response comprises chloride channel activation and the change in second messenger response is a smaller increase in the level of inward chloride current in the presence of both the chemical compound and the second chemical compound than in the presence of only the second chemical compound.

In one embodiment of any of the above methods, the cell is an insect cell. In another embodiment, the cell is a mammalian cell. In another embodiment, the mammalian cell is nonneuronal in origin. In further embodiments, the nonneuronal cell is a COS-7 cell, a 293 human embryonic kidney cell, a CHO cell, a NIH-3T3 cell, a mouse Y1 cell, or a LM(tk−) cell.

For the purposes of this invention, “antagonist potency” is measured as K_{B} which is defined as the equilibrium dissociation constant for the antagonist-receptor complex.

For the purposes of this invention, “agonist potency” is measured as EC50 which is defined as the concentration that is required to elicit 50% of the maximum response in a functional assay.

Throughout the invention, the term “binding affinity” describes the concentration of a compound required to occupy one-half of the binding sites in a receptor population, as detectable by radioligand binding. Binding affinity concentration can be represented as Kᵢ, inhibition constant, or K_{D}, dissociation constant.

The term “selectivity of binding affinity” refers to the ability of a chemical compound to discriminate one receptor from another. For example, a compound showing selectivity for receptor A versus receptor B will bind receptor A at lower concentrations than those required to bind receptor B.

Therefore, the statements of the form “binds to the MCH1 receptor with a binding affinity at least ten-fold higher than” a named receptor, indicates that the binding affinity at the MCH1 receptor is at least ten-fold greater than that for a named receptor, and binding affinity measurements (i.e. Kᵢ or K_{D}) for the compound are at least ten-fold lower in numerical value.

This invention provides a method of treating an eating disorder or obesity in a subject which comprises administering to the subject a therapeutically effective amount of an MCH1 antagonist which inhibits the activation of the MCH1 receptor. In an embodiment, the MCH1 antagonist additionally inhibits the activation of the MCH1 receptor with an antagonist potency which is at least 30-fold greater than the antagonist potency with which the MCH1 antagonist inhibits the activation of each of the 5-HT2C and MC-4 receptors.

In a further embodiment, the MCH1 antagonist additionally inhibits the activation of the MCH1 receptor with an antagonist potency which is at least 10-fold greater than the antagonist potency with which the MCH1 antagonist inhibits the activation of each of the NPY1, NPY5, GALR1, GALR2, and GALR3 receptors. In another embodiment, the MCH1 antagonist additionally inhibits the activation of the MCH1 receptor with an antagonist potency which is at least 100-fold greater than the antagonist potency with which the MCH1 antagonist inhibits the activation of each of the 5-HT2C and MC-4 receptors.

In an additional embodiment, the MCH1 antagonist additionally inhibits the activation of the MCH1 receptor with an antagonist potency which is at least 100-fold greater than the antagonist potency with which the MCH1 antagonist inhibits the activation of each of the NPY1, NPY5, GALR1, GALR2, and GALR3 receptors. In an embodiment, the MCH1 antagonist additionally inhibits the activation of the MCH1 receptor with an antagonist potency which is at least 30-fold greater than the binding affinity with which the MCH1 antagonist binds to each of the 5-HT2C and MC-4 receptors.

In another embodiment, the MCH1 antagonist additionally inhibits the activation of the MCH1 receptor with an antagonist potency which is at least 10-fold greater than the binding affinity with which the MCH1 antagonist binds to each of the NPY1, NPY5, GALR1, GALR2, and GALR3 receptors. In a further embodiment, the MCH1 antagonist additionally inhibits the activation of the MCH1 receptor with an antagonist potency which is at least 100-fold greater than the binding affinity with which the MCH1 antagonist binds to each of the 5-HT2C and MC-4 receptors.

In an additional embodiment, the MCH1 antagonist additionally inhibits the activation of the MCH1 receptor with an antagonist potency which is at least 100-fold greater than the binding affinity with which the MCH1 antagonist binds to each of the NPY1, NPY5, GALR1, GALR2, and GALR3 receptors. In yet another embodiment, the MCH1 antagonist additionally binds to the MCH1 receptor with a binding affinity which is at least 30-fold greater than the binding affinity with which the MCH1 antagonist binds to each of the 5-HT2C and MC-4 receptors. In still another embodiment, the MCH1 antagonist additionally binds to the MCH1 receptor with a binding affinity which is at least 10-fold greater than the binding affinity with which the MCH1 antagonist binds to each of the NPY1, NPY5, GALR1, GALR2, and GALR3 receptors.

In a further embodiment, the MCH1 antagonist additionally binds to the MCH1 receptor with a binding affinity which is at least 100-fold greater than the binding affinity with which the MCH1 antagonist binds to each of the 5-HT2C and MC-4 receptors. In an additional embodiment, the MCH1 antagonist additionally binds to the MCH1 receptor with a binding affinity which is at least 100-fold greater than the binding affinity with which the MCH1 antagonist binds to each of the NPY1, NPY5, GALR1, GALR2, and GALR3 receptors.

In another embodiment, the MCH1 antagonist additionally binds to the MCH1 receptor with a binding affinity which is at least 30-fold greater than the binding affinity with which the MCH1 antagonist binds to the dopamine D2 receptor. In another embodiment, the MCH1 antagonist additionally binds to the MCH1 receptor with a binding affinity which is at least 30-fold greater than the binding affinity with which the MCH1 antagonist binds to the histamine Hi receptor.

In still other embodiments, the MCH1 antagonist additionally binds to the MCH1 receptor with a binding affinity which is at least 100-fold greater than the binding affinity with which the MCH1 antagonist binds the dopamine D2 receptor. In another embodiment, the MCH1 antagonist additionally binds to the MCH1 receptor with a binding affinity which is at least 100-fold greater than the binding affinity with which the MCH1 antagonist binds to the H1 histamine receptor.

In another embodiment, the MCH1 antagonist additionally binds to the MCH1 receptor with a binding affinity which is at least 200-fold greater than the binding affinity with which the MCH1 antagonist binds the dopamine D2 receptor. In still another embodiment, the MCH1 antagonist additionally binds to the MCH1 receptor with a binding affinity which is at least 200-fold greater than the binding affinity with which the MCH1 antagonist binds to the H1 histamine receptor.

In further embodiments, the MCH1 antagonist additionally binds to the MCH1 receptor with a binding affinity which is at least 10-fold greater than the binding affinity with which the MCH1 antagonist binds to the α_{1A} adrenoceptor. In another embodiment, the MCH1 antagonist additionally binds to the MCH1 receptor with a binding affinity which is at least 100-fold greater than the binding affinity with which the MCH1 antagonist binds to the α_{1A} adrenoceptor.

In other embodiments, the MCH1 antagonist additionally binds to the α_{1A} adrenoceptor with a binding affinity which is no more than 10-fold greater than the binding affinity with which the MCH1 antagonist binds to the MCH1 receptor.In still other embodiments, the MCH1 antagonist additionally binds to the α_{1A} adrenoceptor with a binding affinity which is no more than 100-fold greater than the binding affinity with which the MCH1 antagonist binds to the MCH1 receptor.

In any of the embodiments of the present invention, the eating or feeding disorder is bulimia, obesity or bulimia nervosa. In one embodiment, the subject is a vertebrate, a mammal, a human or a canine. In another embodiment, the MCH1 antagonist is administered in combination with food.

This invention also provides a method of treating an eating disorder in a subject which comprises administering to the subject a therapeutically effective amount of an MCH1 agonist which activates the MCH1 receptor. In one embodiment, the MCH1 agonist additionally activates the MCH1 receptor with an agonist potency which is at least 30-fold greater than the agonist potency with which the MCH1 agonist activates each of the 5-HT2C and MC-4 receptors.

In another embodiment, the MCH1 agonist additionally activates the MCH1 receptor with an agonist potency which is at least 10-fold greater than the agonist potency with which the MCH1 agonist activates each of the NPY1, NPY5, GALR1, GALR2, and GALR3 receptors. In a further embodiment, the MCH1 agonist additionally activates the MCH1 receptor with an agonist potency which is at least 100-fold greater than the agonist potency with which the MCH1 agonist activates each of the 5-HT2C and MC-4 receptors.

In yet another embodiment, the MCH1 agonist additionally activates the MCH1 receptor with an agonist potency which is at least 100-fold greater than the agonist potency with which the MCH1 agonist activates each of the NPY1, NPY5, GALR1, GALR2, and GALR3 receptors. In further embodiments, the eating disorder is anorexia nervosa. In another embodiment, the subject is a vertebrate, a mammal, a human or a canine. In a final embodiment, the MCH1 agonist is administered in combination with food.

In the subject invention a “therapeutically effective amount” is any amount of a compound which, when administered to a subject suffering from a disease against which the compounds are effective, causes reduction, remission, or regression of the disease. In the subject application, a “subject” is a vertebrate, a mammal, a human or a canine.

This invention further provides a method of modifying feeding behavior of a subject which comprises administering to the subject an amount of a compound of the present invention effective to decrease the consumption of food by the subject and/or decrease the body mass of the subject. In one embodiment, the subject is a vertebrate, a mammal, a human or a canine. In another embodiment, the MCH1 antagonist is administered in combination with food.

The present invention includes within its scope prodrugs of the compounds of the invention. In general, such prodrugs will be functional derivatives of the compounds of the invention which are readily convertible in vivo into the required compound. Thus, in the present invention, the term “administering” shall encompass the treatment of the various conditions described with the MCH1 antagonist specifically disclosed or with a compound which may not be specifically disclosed, but which converts to the specified MCH1 antagonist in vivo after administration to the patient. Conventional procedures for the selection and preparation of suitable prodrug derivatives are described, for example, in Design of Prodrugs, ed. H. Bundgaard, Elsevier, 1985.

The present invention provides a method of treating depression and/or anxiety in a subject which comprises administering to the subject a composition comprising a pharmaceutically acceptable carrier and a therapeutically effective amount of a MCH1 antagonist, wherein:

(a) (1) the MCH1 antagonist does not inhibit the activity of central monoamine oxidase A greater than 50 percent, at a concentration of 10 mM; and (2) the MCH1 antagonist does not inhibit the activity of central monoamine oxidase B greater than 50 percent, at a concentration of 10 mM; and

(b) the MCH1 antagonist binds to the MCH1 receptor with a binding affinity at least ten-fold higher than the binding affinity with which it binds to each of the following transporters: serotonin transporter, norepinephrine transporter, and dopamine transporter.

For the purposes of this invention the term “pharmaceutically acceptable carrier” has been defined herein.

In other embodiments, the MCH1 antagonist also binds to the MCH1 receptor with a binding affinity at least ten-fold higher than the binding binding affinity with which it binds to each of the human 5HT_{1A}, human 5HT_{1B}, human 5HT_{1D}, human 5HT_{1E}, human 5HT_{1F}, human 5HT_{2A}, rat 5HT_{2C}, human 5HT₄, human 5HT₆ and human 5HT₇ receptors.

In still another embodiment, the MCH1 antagonist also binds to the MCH1 receptor with a binding affinity at least ten-fold higher than the binding affinity with which it binds to the human histamine H₁ and H₂ receptors.

In still another embodiment, the MCH1 antagonist also binds to the MCH1 receptor with a binding affinity at least ten-fold higher than the binding affinity with which it binds to the human dopamine D₁, D₂, D₃, D₄ and D₅ receptors.

In a further embodiment, the MCH1 antagonist also binds to the MCH1 receptor with a binding affinity at least ten-fold higher than the binding affinity with which it binds to the human α_{1A} adrenoceptor, the human α_{1B} adrenoceptor and the human a_{1D} adrenoceptor.

In another embodiment, the MCH1 antagonist also binds to the MCH1 receptor with a binding affinity at least ten-fold higher than the binding affinity with which it binds to the human α_{2A} adrenoceptor, the human α_{2B} adrenoceptor and the human α_{2C} adrenoceptor.

In some embodiments the MCH1 antagonist does not inhibit the activity of central monoamine oxidase A greater than 60 percent. In further embodiments the MCH1 antagonist does not inhibit the activity of central monoamine oxidase B greater than 60 percent. In other embodiments the MCH1 antagonist does not inhibit the activity of central monoamine oxidase A greater than 70 percent. In still other embodiments the MCH1 antagonist does not inhibit the activity of central monoamine oxidase B greater than 70 percent.

The binding properties of compounds at different receptors were determined using cultured cell lines that selectively express the receptor of interest. Cell lines were prepared by transfecting the cloned cDNA or cloned genomic DNA or constructs containing both genomic DNA and cDNA encoding the receptors as further described in the Experimental Details herein below. Furthermore, the binding interactions of compounds at different transporters and enzymes can be determined using tissue preparations and specific assays well known in the art.

In connection with this invention, a number of cloned receptors discussed herein, as stably transfected cell lines, have been made pursuant to, and in satisfaction of, the Budapest Treaty on the International Recognition of the Deposit of Microorganisms for the Purpose of Patent Procedure, and are made with the American Type Culture Collection, 10801 University Blvd., Manassas, Va. 20110-2209. Specifically, these deposits have been accorded ATCC Accession Numbers as follows:

ATCC Deposits:

| | | ATCC | Date |
|---|---|---|---|
| | | Accession | of |
| Designation | Receptor | No. | Deposit |
| | human GAL1 | CRL-1650 | |
| (CHO)hGalR | human GAL2 | CRL 12379 | Jul. 22, 1997 |
| 2-264 | | | |
| L-hGalR3-228 | human GAL3 | CRL-12373 | Jul. 1, 1997 |
| 5HT1A-3 | human 5-HT_{1A} | CRL 11889 | May 11, 1995 |
| Ltk-11 | human 5-HT_{1B} | CRL 10422 | Apr. 17, 1990 |
| | (formerly | | |
| | human | | |
| | 5-HT1D2) | | |
| Ltk-8-30-84 | human 5-HT_{1D} | CRL 10421 | Apr. 17, 1990 |
| | (formerly | | |
| | human | | |
| | 5-HT1D1) | | |
| 5HT_{1E}-7 | human 5-HT_{1E} | CRL 10913 | Nov. 6, 1991 |
| L-5-HT_{1F} | human 5-HT_{1F} | CRL 10957 | Dec. 27, 1991 |
| L-NGC-5HT₂ | human 5- | CRL 10287 | Oct. 31, 1989 |
| | HT_{2A} (formerly | | |
| | human | | |
| | 5-HT2) | | |
| pSr-1c | rat 5-HT_{2C} | 67636 | |
| | (formerly | | |
| | rat | | |
| | 5HT1C) | | |
| pBluescript- | human 5-HT₄ | 75392 | Dec. 22, 1992 |
| hS10 | | | |
| L-5HT-4B | human 5-NT₇ | CRL 11166 | Oct. 20, 1992 |
| | (formerly | | |
| | human 5- | | |
| | HT4B) | | |
| L-α_{1C} | human α_{1A} | CRL11140 | Sep. 25, 1992 |
| | (formerly | | |
| | human α1C) | | |
| L-α_{1B} | human α_{1B} | CRL11139 | Sep. 25, 1992 |
| L-α_{1A} | human α_{1D} | CRL11138 | Sep. 25, 1992 |
| | (formerly | | |
| | hum α1A) | | |
| L-α_{2A} | human α_{2A} | CRL11180 | Nov. 6, 1992 |
| L-NGC-α_{2B} | human α_{2B} | CRL10275 | Oct. 25, 1989 |
| L-α_{2C} | human α_{2C} | CRL11181 | Nov. 6, 1992 |
| pDopD₁-GL-30 | human D₅ | 40839 | Jul. 10, 1990 |
| | (formerly | | |
| | hum D1β) | | |
| pCEXV-H₁ | human H₁ | 75346 | Nov. 6, 1992 |

The “5-HT_{1C}”, “5-HT_{1D1}”, “5-HT_{1D2”, “5-HT}_{4B}”, and “5HT₂” receptors were renamed the “5-HT_{2C}”, 5-HT_{1D}”“5-HT_{1B}”, “5-HT₇”, and “5-HT_{2A}” receptors, respectively, by the Serotonin Receptor Nomenclature Committee of the IUPHAR.
The “human α_{1C}”, “human α_{1A}”, and “human D_{1β}” were renamed the “human α_{1A}”, “human α_{1D}”, and “human D₅” respectively.

The following receptor sequences have been deposited with the GenBank DNA database, which is managed by the National Center for Biotechnology (Bethesda, Md.).

GENBANK DEPOSITS

| DESIGNATION | RECEPTOR | GENBANK No. |
|---|---|---|
| human mRNA for | human D₁ | X58987 |
| D-1 receptor | (formerly human D_{1α}) | |
| human dopamine | human D₂ | M29066 |
| D2 receptor | | |
| (DRD2) mRNA | | |
| complete cds | | |
| Rat mRNA for | rat D₃ | X53944 |
| dopamine D3 | | |
| receptor | | |
| Homo sapiens | human D₄ | L12397 |
| dopamine D4 | | |
| receptor | | |
| (DRD4) gene | | |
| (D4.4) | | |
| sequence | | |

* The “human D_{1α}” receptor was renamed the “human D₁” receptor.

Thus, once the gene for a targeted receptor subtype is cloned, it is placed into a recipient cell which then expresses the targeted receptor subtype on its surface. This cell, which expresses a single population of the targeted human receptor subtype, is then propagated resulting in the establishment of a cell line. This cell line, which constitutes a drug discovery system, is used in two different types of assays: binding assays and functional assays. In binding assays, the affinity of a compound for both the receptor subtype that is the target of a particular drug discovery program and other receptor subtypes that could be associated with side effects are measured. These measurements enable one to predict the potency of a compound, as well as the degree of selectivity that the compound has for the targeted receptor subtype over other receptor subtypes. The data obtained from binding assays also enable chemists to design compounds toward or away from one or more of the relevant subtypes, as appropriate, for optimal therapeutic efficacy. In functional assays, the nature of the response of the receptor subtype to the compound is determined. Data from the functional assays show whether the compound is acting to inhibit or enhance the activity of the receptor subtype, thus enabling pharmacologists to evaluate compounds rapidly at their ultimate human receptor subtypes targets permitting chemists to rationally design drugs that will be more effective and have fewer or substantially less severe side effects than existing drugs.

Approaches to designing and synthesizing receptor subtype-selective compounds are well known and include traditional medicinal chemistry and the newer technology of combinatorial chemistry, both of which are supported by computer-assisted molecular modeling. With such approaches, chemists and pharmacologists use their knowledge of the structures of the targeted receptor subtype and compounds determined to bind and/or activate or inhibit activation of the receptor subtype to design and synthesize structures that will have activity at these receptor subtypes.

Combinatorial chemistry involves automated synthesis of a variety of novel compounds by assembling them using different combinations of chemical building blocks. The use of combinatorial chemistry greatly accelerates the process of generating compounds. The resulting arrays of compounds are called libraries and are used to screen for compounds (“lead compounds”) that demonstrate a sufficient level of activity at receptors of interest. Using combinatorial chemistry it is possible to synthesize “focused” libraries of compounds anticipated to be highly biased toward the receptor target of interest.

Once lead compounds are identified, whether through the use of combinatorial chemistry or traditional medicinal chemistry or otherwise, a variety of homologs and analogs are prepared to facilitate an understanding of the relationship between chemical structure and biological or functional activity. These studies define structure activity relationships which are then used to design drugs with improved potency, selectivity and pharmacokinetic properties. Combinatorial chemistry is also used to rapidly generate a variety of structures for lead optimization. Traditional medicinal chemistry, which involves the synthesis of compounds one at a time, is also used for further refinement and to generate compounds not accessible by automated techniques. Once such drugs are defined the production is scaled up using standard chemical manufacturing methodologies utilized throughout the pharmaceutical and chemistry industry.

This invention will be better understood from the Experimental Details which follow. However, one skilled in the art will readily appreciate that the specific methods and results discussed are merely illustrative of the invention as described more fully in the claims which follow thereafter.

### EXPERIMENTAL DETAILS

Materials and Methods

Cloning of Human MCH1 Receptor

Discovery of an Expressed Sequence Tag (EST) F07228 in GENEMBL Homologous to FB41a

A BLAST search of GENEMBL was performed with the GCG sequence analysis package (Genetics Computer Group, Madison, Wis.) using a Synaptic Pharmaceutical Corporation proprietary sequence, FB41a, as a query. This resulted in the identification of an EST (accession number F07228) with a high degree of homology to FB41a and somatostatin, opiate and galanin receptors.

Construction and Screening of a Human Hippocampal cDNA Library

Poly A+ RNA was purified from human hippocampal RNA (Clontech) using a FastTrack kit (Invitrogen, Corp.). DS-cDNA was synthesized from poly A+ RNA according to Gubler and Hoffman (1983) with minor modifications. The resulting cDNA was ligated to BstXI adaptors (Invitrogen, Corp.) and the excess adaptors removed by exclusion column chromatography. High molecular weight fractions of size-selected ds-cDNA were ligated in pEXJ.BS, an Okayama and Berg expression vector modified from pcEXV (Miller and Germain, 1986) to contain BstXI and other additional restriction sites. A total of 2.2×10⁶independent clones with a mean insert size of 3.0 kb were generated. The library was plated on agar plates (ampicillin selection) and glycerol stocks for 450 pools of 5000 independent clones were prepared. Primary glycerol stocks were also grouped together in groups of approximately 10 to create superpools.

Cloning of the Full-Length Sequence of MCH1

Glycerol stocks of the superpools and primary pools from the human hippocampal cDNA library were screened by PCR with F07228 specific primers T579 and T580 using Taq DNA Polymerase (Boehringer-Mannheim, Indianapolis, Ind.) and the following PCR protocol: 94° C. hold for 5 minutes; 40 cycles of 94° C. for 2 minute, 68° C. for 4 minutes; 7 minute hold at 68° C.; 4° C. hold until the samples are run on a gel. One positive primary pool 490, was successively divided into subpools, amplified in LB medium overnight and screened by PCR using primers T579 and T580. One positive subpool, 490-4-10-23 was plated on agar plates (ampicillin selection), and colonies were transferred to nitrocellulose membranes (Schleicher and Schuell, Keene, N.H.). Filters were hybridized for two days under high stringency conditions with 10⁶ cpm/ml of a ³²P-labeled cDNA probe, T581, designed against the F07228 EST sequence. Filters were washed and apposed to Biomax MS film (Kodak). Seven positive colonies were picked, streaked on LB-AMP plates, and grown overnight. Two individual colonies from each of the original seven were picked and subjected to vector-anchored PCR using the following primer pairs: T95, T580 and T94, T579. One positive colony, G1, was amplified overnight in TB and processed for plasmid purification. This plasmid was designated TL230 and sequenced on both strands with a Sequenase kit (US Biochemical, Cleveland, Ohio). Nucleotide and peptide sequence analysis were performed with GCG programs (Genetics Computer Group, Madison, Wis.). A HindIII-KpnI fragment of TL230 was subcloned into the mammalian expression vector pEXJ, and named TL231.

| | |
|---|---|
| Primers and Probes: | |
| TL579: | |
| 5′-GGGAACTCCACGGTCATCTTCGCGGT-3′ | (SEQ ID NO:5) |
| TL580: | |
| 5′-TAGCGGTCAATGGCCATGGCGGTCAG-3′ | (SEQ ID NO:6) |
| TL5B1: | |
| 5′-CTCCTGGGCATGCCCTTCATGATCCACCAGCTCA | (SEQ ID NO:7) |
| TGGGCAATGGG-3′ | |
| TL94: | |
| 5′-CTTCTAGGCCTGTACGGAAGTGTTA-3′ | (SEQ ID NO:8) |
| TL95: | |
| 5′-GTTGTGGTTTGTCCAAACTCATCAATG-3′ | (SEQ ID NO:9) |

Isolation of a Fragment of a Species Homologue of TL231 (Human MCH1)

To obtain a fragment of a species homologue of TL231, the species genomic DNA (Clontech) may be amplified with a forward PCR primer corresponding to one of the TM regions of TL231 and a reverse primer corresponding to another TM region of TL231. PCR may be performed with the Expand Long Template PCR System (Boeringer Mannheim), for example, under the following conditions: 30 sec at 94° C., 1.5 min at 50° C., 1.5 min at 68° C. for 40 cycles, with a pre- and post-incubation of 5 min at 94° C. and 7 min at 68° C., respectively. A band is isolated, subcloned using the TA cloning kit (Invitrogen), and sequenced. The sequence is run and analyzed on an ABI PRISM 377 BigDye Terminator Cycle Sequencing Kit Sequencer. Forward and reverse PCR primers are designed against this sequence and used to amplify a band from genomic DNA using, for example, the following conditions: 30 sec at 94° C., 1.5 min at 68° C. for 35 cycles, with a pre- and post-incubation of 5 min at 94° C. and 5 min at 68° C., respectively. The PCR product is subcloned using the TA cloning kit (Invitrogen). Miniprep cultures of transformants are prepared and sequenced as above.

Isolation of a Full-Length Species Homolog of TL231 (Human MCH1)

A nucleic acid sequence encoding an MCH1 receptor may be isolated using standard molecular biology techniques and approaches such as those briefly described below:

Approach #1: To obtain a full-length MCH1 receptor, a cosmid library could be screened with a ³²P-labeled oligonucleotide probe.

The full-length sequence may be obtained by sequencing this cosmid clone with additional sequencing primers. Since one intron is present in this gene the full-length intronless gene may be obtained from cDNA using standard molecular biology techniques. For example, a forward PCR primer designed in the 5′UT and a reverse PCR primer designed in the 3′UT may be used to amplify a full-length, intronless gene from cDNA. Standard molecular biology techniques could be used to subclone this gene into a mammalian expression vector.

Approach #2: Standard molecular biology techniques could be used to screen commercial cDNA phage libraries by hybridization under high stringency with a ³²P-labeled oligonucleotide probe. One may isolate a full-length MCH1 receptor by obtaining a plaque purified clone from the lambda libraries and then subjecting the clone to direct DNA sequencing. Alternatively, standard molecular biology techniques could be used to screen in-house cDNA plasmid libraries by PCR amplification of library pools using primers to the MCH1 sequence. A full-length clone could be isolated by Southern hybridization of colony lifts of positive pools with a ³²P-labeled oligonucleotide probe.

Approach #3: As yet another alternative method, one could utilize 3′ and 5′ RACE to generate PCR products from cDNA expressing MCH1 which contain the additional sequences of MCH1. These RACE PCR products could then be sequenced to determine the missing sequence. This new sequence could then be used to design a forward PCR primer in the 5′UT and a reverse primer in the 3′UT. These primers could then be used to amplify a full-length MCH1 clone from cDNA.

Construction of Human MCH1 Mutants

The plasmid TL231 encodes three in frame methionine residues, any of which could potentially initiate translation of the MCH1 receptor. The ability of these residues to function in a heterologous expression system was examined by constructing mutants of TL231 in which one or more of the downstream methionine residues was mutated to alanine. Mutagenesis was performed using the QuickChange site-directed mutagenesis kit (Stratagene). Each 50 ul PCR reaction contained 10 mM KCl, 10 mM (NH₄)₂SO₄, 20 mM Tris-HCl (pH 8.8), 2 mM MgSO₄, 0.1% Triton X-100, 0.1 mg/ml nuclease-free BSA, 114 ng each of two mutagenesis primers (see below), 50 ng of plasmid DNA template (see below), 2.5 units of PfuTurbo DNA polymerase, and 1 ul of the proprietary dNTP mix provided in the kit. Thermocycling was performed with an Applied Biosystems 9700 machine using the following cycling parameters: one cycle of 95° for 30 seconds; eighteen cycles of 95° for 30 seconds, 55° for 1 minute, 680 for 2.5 minutes; a final hold at 4°. Next, 1 ul (10 units) of DpnI restriction enzyme was added to the mutagenesis reaction followed by incubation at 37° for 1 hour. A 2 ul aliquot of this digestion was used to transform 50 ul of E.coli XL1-Blue cells provided with the mutagenesis kit. Transformants were selected by their ability to grow at 37° on LB plates containing 100 ug/ml ampicillin. Single colonies which resulted from the overnight incubation of the plates were used to inoculate 2 ml cultures of LB-ampicillin and allowed to grow overnight at 37° with shaking. Miniprep DNA was prepared from these cultures using the Qiagen miniprep system and subjected to automated sequence analysis. This allowed both the confirmation of the desired mutation and the integrity of the remainder of the MCH1 coding sequence. After identification of a correctly mutated clone, a large scale DNA prep was prepared using a Qiagen megaprep column.

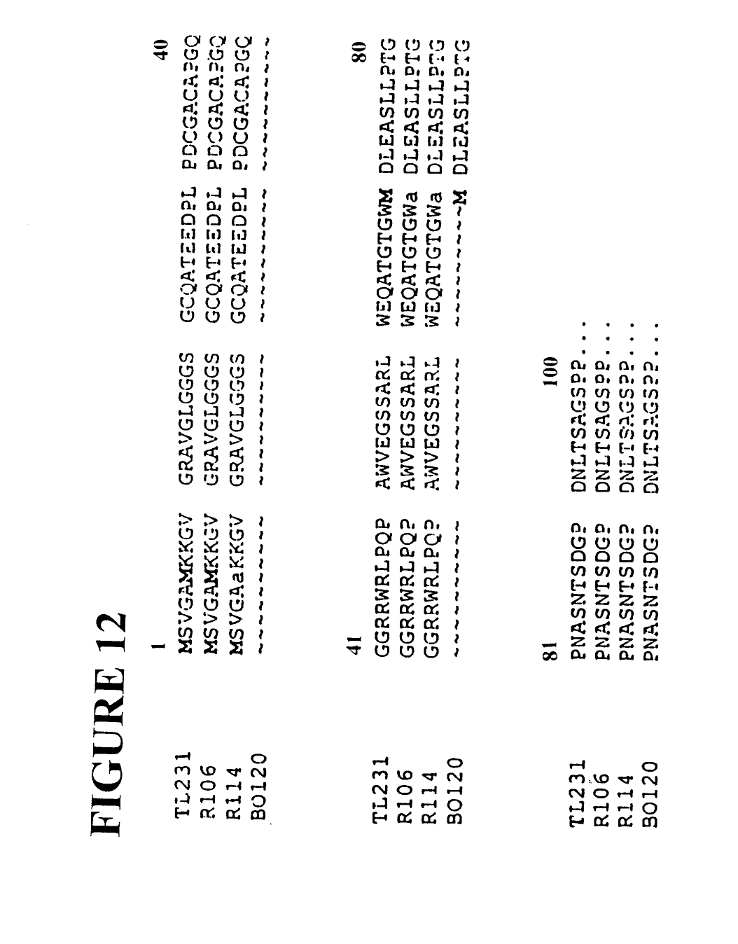

To create the clone encoding only the M70A mutation, the template DNA was TL231 and the mutagenesis primers were RP192 and RP193. This clone is designated R106 (SEQ ID NO: 16) and encodes only the first two potential start codons (See FIG. 12). To create the clone encoding both the M6A and the M70A mutations, the template DNA was R106 and the mutagenesis primers were RP190 and RP191. The resulting clone is designated R114 (SEQ ID NO: 17) and encodes only first start codon (See FIG. 12).

If desired, the same mutagenesis technology can be employed to construct additional MCH1 mutants that encode other combinations of the available methionine residues. The mutation M1A could be constructed using primers X1 and X2. Such a change would eliminate the first methionine but retain the two downstream residues. Likewise, the double mutation M1A, M70A could be constructed by sequentially using primer pairs X1/X2 and RP192/RP193. This would create a gene in which only the second methionine was left intact.

Primers used in the generation of hMCH1 mutant receptor constructs:

| Mutant | Primer | Primer Sequence | |
|---|---|---|---|
| R106 | RP192 | 5′ CGGCACTGGCTGGGCGGAC | (SEQ ID NO:18) |
| | | CTGGAAGCCTCG 3′ | |
| M70A) | RP193 | 5′ CGAGGCTTCCAGGTCCGCC | (SEQ ID NO:19) |
| | | CAGCCAGTGCCG 3′ | |
| R114 | RP190 | 5′ ATGTCAGTGGGAGCCGCGA | (SEQ ID NO:20) |
| | | AGAAGGGAGTGGG 3′ | |
| (M6A, | RP191 | 5′ CCCACTCCCTTCTTCGCGG | (SEQ ID NO:21) |
| M70A) | | CTCCCACTGACAT 3′ | |
| (M1A) | X1 | 5′ TAATGTGTCTAGGTGGCGT | (SEQ ID NO:22) |
| | | CAGTGGGAGCCATG 3′ | |
| | X2 | 5′ CATGGCTCCCACTGACGCC | (SEQ ID NO:23) |
| | | ACCTAGACACATTA 3′ | |

Construction of a Short form of the Human MCH1 Receptor

A short form of the human MCH1 receptor expressing only the most downstream of the three potential initiating methionines was generated as follows. TL231 was amplified with BB1122 (a forward primer beginning 10 nucleotides upstream of the third methionine in TL231, and also incorporating a HindIII site) and BB1123 (a reverse primer in the second transmembrane domain) and the resulting product digested with HindIII and BglIIA. PCR was performed with the Expand Long Template PCR System (Roche Molecular Biochemicals, Indianapolis, Ind.) under the following conditions: 20 seconds at 94° C., 1 minute at 68° C. for 40 cycles, with a pre- and post-incubation of 5 minutes at 94° C. and 7 minutes at 68° C. respectively. The 270 bp product was gel purified and ligated to a 4 kb HindIII/BglII restriction fragment from TL231. The resulting construct was named BO120.

Primers used in the construction of the truncated human MCH1 receptor:

| | |
|---|---|
| BB1122 | |
| 5′- TGACACTAAGCTTCACTGGCTGGATGGACCTG | (SEQ ID NO:24) |
| GAAGC -3′ | |
| BB1123 | |
| 5′- GCCCAGGAGAAAGAGGAGATCTAC -3′ | (SEQ ID NO:25) |

Host Cells

A broad variety of host cells can be used to study heterologously expressed proteins. These cells include but are not restricted to assorted mammalian lines such as; Cos-7, CHO, LM(tk−), HEK293, etc.; insect cell lines such as; Sf9, Sf21, etc.; amphibian cells such as xenopus oocytes; and others.

COS-7 cells are grown on 150 mm plates in DMEM with supplements (Dulbecco's Modified Eagle Medium with 10% bovine calf serum, 4 mM glutamine, 100 units/ml penicillin/100 μg/ml streptomycin) at 37° C., 5% CO₂. Stock plates of COS-7 cells are trypsinized and split 1:6 every 3-4 days.

Human embryonic kidney 293 cells are grown on 150 mm plates in DMEM with supplements (10% bovine calf serum, 4 mM glutamine, 100 units/ml penicillin/100 μg/ml streptomycin) at 37° C., 5% CO₂. Stock plates of 293 cells are trypsinized and split 1:6 every 3-4 days.

Mouse fibroblast LM(tk−) cells are grown on 150 mm plates in D-MEM with supplements (Dulbecco's Modified Eagle Medium with 10% bovine calf serum, 4 mM glutamine, 100 units/ml penicillin/10μg/ml streptomycin) at 37° C., 5% CO₂. Stock plates of LM(tk−) cells are trypsinized and split 1:10 every 3-4 days.

Chinese hamster ovary (CHO) cells were grown on 150 mm plates in HAM's F-12 medium with supplements (10% bovine calf serum, 4 mM L-glutamine and 100 units/ml penicillin/100 μg/ml streptomycin) at 37° C., 5% CO₂. Stock plates of CHO cells are trypsinized and split 1:8 every 3-4 days. Mouse embryonic fibroblast NIH-3T3 cells are grown on 150 mm plates in Dulbecco's Modified Eagle Medium (DMEM) with supplements (10% bovine calf serum, 4 mM glutamine, 100 units/ml penicillin/100 μg/ml streptomycin) at 37° C., 5% CO₂. Stock plates of NIH-3T3 cells are trypsinized and split 1:15 every 3-4 days.

Sf9 and Sf21 cells are grown in monolayers on 150 mm tissue culture dishes in TMN-FH media supplemented with 10% fetal calf serum, at 27° C., no CO₂. High Five insect cells are grown on 150 mm tissue culture dishes in Ex-Cell 400™ medium supplemented with L-Glutamine, also at 27° C., no CO₂.

In some cases, cell lines that grow as adherent monolayers can be converted to suspension culture to increase cell yield and provide large batches of uniform assay material for routine receptor screening projects.

Xenopus oocytes can also be used as a host system for transient expression of heterologous proteins. Their maintenance and usage is described in the electrophysiological methods section that follows.

Transient Expression

DNA encoding proteins to be studied can be transiently expressed in a variety of mammalian, insect, amphibian and other cell lines by several methods including but not restricted to; calcium phosphate-mediated, DEAE-dextran mediated, Liposomal-mediated, viral-mediated, electroporation-mediated and microinjection delivery. Each of these methods may require optimization of assorted experimental parameters depending on the DNA, cell line, and the type of assay to be subsequently employed.

A typical protocol for the calcium phosphate method as applied to LM(tk−) cells is described as follows; Adherent cells are harvested approximately twenty-four hours before transfection and replated at a density of 1-2×10⁵ cells/cm² in a 100 mm tissue culture dish and allowed to incubate over night at 37° C. at 5% CO₂. 250 μl of a mixture of CaCl₂ and DNA (20 μg DNA in 250 mM CaCl₂) is added to a 5 ml plastic tube and 250 ul of 2× HBS (250 mM NaCl, 10 mM KCl, 1.5 mM Na₂HPO₄, 12 mM dextrose, 50 mM HEPES) is slowly added with gentle mixing. The mixture is allowed to incubate for 20 minutes at room temperature to allow a DNA precipitate to form. The cells are then washed with complete medium, 10 ml of culture medium is added to each plate, followed by addition of the DNA precipitate. The cells are then incubated for 24 to 48 hours at 37° C. at 5% CO₂.

A typical protocol for the DEAE-dextran method as applied to Cos-7 cells is described as follows; Cells to be used for transfection are split 24 hours prior to the transfection to provide flasks which are 70-80% confluent at the time of transfection. Briefly, 8 μg of receptor DNA plus 8 μg of any additional DNA needed (e.g. G_{α} protein expression vector, reporter construct, antibiotic resistance marker, mock vector, etc.) are added to 9 ml of complete DMEM plus DEAE-dextran mixture (10 mg/ml in PBS). Cos-7 cells plated into a T225 flask (sub-confluent) are washed once with PBS and the DNA mixture is added to each flask. The cells are allowed to incubate for 30 minutes at 37° C., 5% CO₂. Following the incubation, 36 ml of complete DMEM with 80 μM chloroquine is added to each flask and allowed to incubate an additional 3 hours. The medium is then aspirated and 24 ml of complete medium containing 10% DMSO for exactly 2 minutes and then aspirated. The cells are then washed 2 times with PBS and 30 ml of complete DMEM added to each flask. The cells are then allowed to incubate over night. The next day the cells are harvested by trypsinization and reseeded as needed depending upon the type of assay to be performed.

A typical protocol for liposomal-mediated transfection as applied to CHO cells is described as follows; Cells to be used for transfection are split 24 hours prior to the transfection to provide flasks which are 70-80% confluent at the time of transfection. A total of 10 μg of DNA which may include varying ratios of receptor DNA plus any additional DNA needed (e.g. G_{α} protein expression vector, reporter construct, antibiotic resistance marker, mock vector, etc.) is used to transfect each 75 cm² flask of cells. Liposomal mediated transfection is carried out according to the manufacturer's recommendations (LipofectAMINE, GibcoBRL, Bethesda, Md.). Transfected cells are harvested 24 h post transfection and used or reseeded according the requirements of the assay to be employed.

A typical protocol for the electroporation method as applied to Cos-7 cells is described as follows; Cells to be used for transfection are split 24 hours prior to the transfection to provide flasks which are subconfluent at the time of transfection. The cells are harvested by trypsinization resuspended in their growth media and counted. 4×10⁶ cells are suspended in 300 μl of DMEM and placed into an electroporation cuvette. 8 μg of receptor DNA plus 8 μg of any additional DNA needed (e.g. G, protein expression vector, reporter construct, antibiotic resistance marker, mock vector, etc.) is added to the cell suspension, the cuvette is placed into a BioRad Gene Pulser and subjected to an electrical pulse (Gene Pulser settings: 0.25 kV voltage, 950 μF capacitance). Following the pulse, 800 μl of complete DMEM is added to each cuvette and the suspension transferred to a sterile tube. Complete medium is added to each tube to bring the final cell concentration to 1×10⁵ cells/100 μl. The cells are then plated as needed depending upon the type of assay to be performed.

A typical protocol for viral mediated expression of heterolgous proteins is described as follows for baculovirus infection of insect Sf9 cells. The coding region of DNA encoding the receptor disclosed herein may be subcloned into pBlueBacIII into existing restriction sites or sites engineered into sequences 5′ and 3′ to the coding region of the polypeptides. To generate baculovirus, 0.5 μg of viral DNA (BaculoGold) and 3 μg of DNA construct encoding a polypeptide may be co-transfected into 2×10⁶ Spodoptera frugiperda insect Sf9 cells by the calcium phosphate co-precipitation method, as outlined in by Pharmingen (in “Baculovirus Expression Vector System: Procedures and Methods Manual”). The cells then are incubated for 5 days at 27° C. The supernatant of the co-transfection plate may be collected by centrifugation and the recombinant virus plaque purified. The procedure to infect cells with virus, to prepare stocks of virus and to titer the virus stocks are as described in Pharmingen's manual. Similar principals would in general apply to mammalian cell expression via retro-viruses, Simliki forest virus and double stranded DNA viruses such as adeno-, herpes-, and vacinia-viruses, and the like.

Microinjection of cRNA encoding for proteins of interest is useful for the study of protein function in xenopus oocytes as well as cultured mammalian cells. A typical protocol for the preparation of cRNA and injection into xenopus oocytes can be found in the following electrophysiology section.

Stable Expression

Heterologous DNA can be stably incorporated into host cells, causing the cell to perpetually express a foreign protein. Methods for the delivery of the DNA into the cell are similar to those described above for transient expression but require the co-transfection of an ancillary gene to confer drug resistance on the targeted host cell. The ensuing drug resistance can be exploited to select and maintain cells that have taken up the heterologous DNA. An assortment of resistance genes are available including but not restricted to Neomycin, Kanamycin, and Hygromycin. For the purposes of receptor studies, stable expression of a heterologous receptor protein is carried out in, but not necessarily restricted to, mammalian cells including, CHO, HEK293, LM(tk−), etc.

Cell Membrane Preparation

For binding assays, pellets of transfected cells are suspended in ice-cold buffer (20 mM Tris.HCl, 5 mM EDTA, pH 7.4) and homogenized by sonication for 7 sec. The cell lysates are centrifuged at 200× g for 5 min at 4° C. The supernatants are then centrifuged at 40,000× g for 20 min at 4° C. The resulting pellets are washed once in the homogenization buffer and suspended in binding buffer (see methods for radioligand binding). Protein concentrations are determined by the method of Bradford (1976) using bovine serum albumin as the standard. Binding assays are usually performed immediately, however it is possible to prepare membranes in batch and store frozen in liquid nitrogen for future use.

Radioligand Binding Assays

Cells may be screened for the presence of endogenous human receptor by radioligand binding (described in detail below). Cells with either no or a low level of the endogenous human receptor disclosed herein may be transfected with the exogenous receptor.

MCH1 binding experiments with membranes (20-40 μg membrane protein) from transfected cells are performed with 0.1 nM [¹²⁵I]Phe¹³-Tyr¹⁹-MCH (Custom labeled by NEN) using incubation buffer consisting of 50 mM Tris pH 7.4, 10 mM MgCl₂, 2 μg/ml aprotonin, 0.5 mM PMSF and 50 μg/ml bacitracin. Binding is performed at 25° C. for 1 hr. Incubations are terminated by rapid vacuum filtration over GF/C glass fiber filters, presoaked in 5% PEI using 50 mM Tris pH 7.4 containing 0.01% triton X-100 as wash buffer. In all experiments nonspecific binding is defined using 10 μM unlabeled MCH.

Functional Assays

Cells may be screened for the presence of endogenous mammalian receptor using functional assays (described in detail below). Cells with no or a low level of endogenous receptor present may be transfected with the exogenous receptor for use in the following functional assays.

A wide spectrum of assays can be employed to screen for receptor activation. These range from traditional measurements of phosphatidyl inositol, cAMP, Ca⁺⁺, and K⁺, for example; to systems measuring these same second messengers but which have been modified or adapted to be higher throughput, more generic, and more sensitive; to cell based platforms reporting more general cellular events resulting from receptor activation such as metabolic changes, differentiation, and cell division/proliferation, for example; to high level organism assays which monitor complex physiological or behavioral changes thought to be involved with receptor activation including cardiovascular, analgesic, orexigenic, anxiolytic, and sedation effects, for example.

Cyclic AMP (cAMP) Assay

The receptor-mediated stimulation or inhibition of cyclic AMP (cAMP) formation may be assayed in cells expressing the mammalian receptors. Cells are plated in 96-well plates and incubated in Dulbecco's phosphate buffered saline (PBS) supplemented with 10 mM HEPES, lmM isobutylmethylxanthine for 20 min at 37° C., in 5% CO₂. Test compounds are added with or without 10 μM forskolin and incubated for an additional 10 min at 37° C. The medium is then aspirated and the reaction stopped by the addition of 100 mM HCl. The plates are stored at 4° C. for 15 min, and the cAMP content in the stopping solution measured by radioimmunoassay. Radioactivity may be quantified using a gamma counter equipped with data reduction software.

Arachidonic Acid Release Assay

Cells expressing the mammalian receptor are seeded into 96 well plates and grown for 3 days in HAM's F-12 with supplements. [³H]-arachidonic acid (specific activity=0.75 μCi/ml) is delivered as a 100 μL aliquot to each well and samples were incubated at 37° C., 5% CO₂ for 18 hours. The labeled cells are washed three times with 200 μL HAM's F-12. The wells are then filled with medium (200 μL) and the assay is initiated with the addition of peptides or buffer (22 μL). Cells are incubated for 30 min at 37° C., 5% CO₂. Supernatants are transferred to a microtiter plate and evaporated to dryness at 75° C. in a vacuum oven. Samples are then dissolved and resuspended in 25 μL distilled water. Scintillant (300 μL) is added to each well and samples are counted for ³H in a Trilux plate reader. Data are analyzed using nonlinear regression and statistical techniques available in the GraphPAD Prism package (San Diego, Calif.).

Intracellular Calcium Mobilization Assay

The intracellular free calcium concentration may be measured by microspectroflourometry using the fluorescent indicator dye Fura-2/AM (Bush et al, 1991). Cells are seeded onto a 35 mm culture dish containing a glass coverslip insert, washed with HBS and loaded with 100 μL of Fura-2/AM (10 μM) for 20 to 40 min. After washing with HBS to remove the Fura-2/AM solution, cells are equilibrated in HBS for 10 to 20 min. Cells are then visualized under the 40× objective of a Leitz Fluovert FS microscope and fluorescence emission is determined at 510 nM with excitation wavelengths alternating between 340 nM and 380 nM. Raw fluorescence data are converted to calcium concentrations using standard calcium concentration curves and software analysis techniques.

Inositol Phosphate Assay

Guidelines for cell preparation and assay of the second messenger inositol phosphate (IP) are described below for a typical protocol involving transiently transfected Cos-7 cells; For a 96 well microplate format assay, cells are plated at 70,000 cells per well and allowed to incubate for 24 hours after the transfection procedure. The cells are then labeled with 0.5 μCi [³H]myo-inositol per micro-well over night at 37° C., 5% CO₂. Immediately before the assay, the medium is removed and replaced with 90 μl PBS containing 10 mM LiCl. The plates are then incubated for 15 minutes at 37° C., 5% CO₂. Following the incubation, the transfectants are challenged with agonist (10 μl/well; 10× concentration) for 30 minutes at 37° C., 5% CO₂. The challenge is terminated and the cells lysed by the addition of 100 μl cold 5% v/v trichloroacetic acid (TCA), followed by an incubation at 4° C. for greater than 30 minutes. Total IPs are isolated from the lysate by ion exchange chromatography. Briefly, the lysed contents of the wells are transferred to a Multiscreen HV filter plate (Millipore) containing 100 μl Dowex AG1-X8 suspension (50% v/v, water:resin) (200-400 mesh, formate form). The filter plates are placed on a vacuum manifold to wash and elute the resin bed. Each well is first washed 2 times with 200 μl 5 mM myoinositol. Total [³H]IPs are eluted with 75 μl of 1.2 M ammonium formate/0.1 M formic acid into Wallac 96-well plates. 200 μl of SuperMix scintillation cocktail is added to each well, mixed well, allowed to equilibrate and counted on a Micro Beta Trilux scintillation counter. (Note: The assay may be scaled to a 24 well format by simple adjustment of reagent volumes and employing individual chromatographic columns.)

GTPγS Functional Assay

Membranes from cells transfected with the mammalian receptors are suspended in assay buffer (50 mM Tris, 100 mM NaCl, 5 MM MgCl₂, pH 7.4) supplemented with 0.2% BSA and 10 μM GDP. Membranes are incubated on ice for 20 minutes, transferred to a 96-well Millipore microtiter GF/C filter plate and mixed with GTPy³⁵S (e.g., 250,000 cpm/sample, specific activity -1000 Ci/mmol) plus or minus GTPγS (final concentration=100 μM). Final membrane protein concentration≈90 μg/ml. Samples are incubated in the presence or absence of MCH (final concentration=1 μM) for 30 min. at room temperature, then filtered on a Millipore vacuum manifold and washed three times with cold assay buffer. Samples collected in the filter plate are treated with scintillant and counted for ³⁵S in a Trilux (Wallac) liquid scintillation counter. It is expected that optimal results are obtained when the mammalian receptor membrane preparation is derived from an appropriately engineered heterologous expression system, i.e., an expression system resulting in high levels of expression of the mammalian receptor and/or expressing G-proteins having high turnover rates (for the exchange of GDP for GTP). GTPγS assays are well-known in the art, and it is expected that variations on the method described above, such as are described by e.g., Tian et al. (1994) or Lazareno and Birdsall (1993), may be used by one of ordinary skill in the art.

Transcription Assay

Guidelines for cell preparation and assay of receptor mediated transcription of Cos-7 cells transiently transfected by the DEAE-dextran method in a 96 microwell format is as follows; The c-fos-β-gal promoter/reporter construct used for these studies consists of the cfos promoter region (−384 to +19) (Schilling et al 1991, Yalkinoglu et al, 1995) inserted upstream of β-galactosidase cDNA containing expression vector pNASSβ (Clontech). Transcription activity is measured by assay of β-galactosidase enzyme activity as detected in a calorimetric assay. Forty-eight hours following transient transfection, the medium is removed and replaced with medium containing drug (e.g. MCH) typically at a concentration of 10 μM. The cells are allowed to incubate at 37° C., 5% CO₂ for at least 18 hours, after which the medium is aspirated and the cells washed with 200 μl PBS/well. The cells are then lysed with 100 μl AB buffer (100 mM Sodium Phosphate buffer, pH 8.0, 2 mM MgSO₄, 0.1 mM MnCl₂) for 10 minutes at room temperature. 100 μl of AB/Tx/β-mercaptoethanol (AB buffer with 0.5% Triton X-100, 40 mM β-mercaptoethanol) is then added to each well and the lysate allowed to incubate an additional 10 minutes at room temperature. The enzymatic color reaction is initiated by the addition of the substrate, ONPG/AB (4 mg/ml O-nitrophenyl-b-D-galactopyranoside in AB buffer) The reaction is allowed to proceed for 30 minutes or until yellow color becomes evident. Measurement of optical density is taken at 405 nm using a Dynatech microplate reader.

MAP Kinase Assay

MAP kinase (mitogen activated kinase) may be monitored to evaluate receptor activation. MAP kinase is activated by multiple pathways in the cell. A primary mode of activation involves the ras/raf/MEK/MAP kinase pathway. Growth factor (tyrosine kinase) receptors feed into this pathway via SHC/Grb-2/SOS/ras. Gi coupled receptors are also known to activate ras and subsequently produce an activation of MAP kinase. Receptors that activate phospholipase C (Gq and G11) produce diacylglycerol (DAG) as a consequence of phosphatidyl inositol hydrolysis. DAG activates protein kinase C which in turn phosphorylates MAP kinase.

MAP kinase activation can be detected by several approaches. One approach is based on an evaluation of the phosphorylation state, either unphosphorylated (inactive) or phosphorylated (active). The phosphorylated protein has a slower mobility in SDS-PAGE and can therefore be compared with the unstimulated protein using Western blotting. Alternatively, antibodies specific for the phosphorylated protein are available (New England Biolabs) which can be used to detect an increase in the phosphorylated kinase. In either method, cells are stimulated with the mitogen and then extracted with Laemmli buffer. The soluble fraction is applied to an SDS-PAGE gel and proteins are transferred electrophoretically to nitrocellulose or Immobilon. Immunoreactive bands are detected by standard Western blotting technique. Visible or chemiluminescent signals are recorded on film and may be quantified by densitometry.

Another approach is based on evaluation of the MAP kinase activity via a phosphorylation assay. Cells are stimulated with the mitogen and a soluble extract is prepared. The extract is incubated at 30° C. for 10 min with gamma-32-ATP, an ATP regenerating system, and a specific substrate for MAP kinase such as phosphorylated heat and acid stable protein regulated by insulin, or PHAS-I. The reaction is terminated by the addition of H₃PO₄ and samples are transferred to ice. An aliquot is spotted onto Whatman P81 chromatography paper, which retains the phosphorylated protein. The chromatography paper is washed and counted for ³²P in a liquid scintillation counter. Alternatively, the cell extract is incubated with gamma-32-ATP, an ATP regenerating system, and biotinylated myelin basic protein bound by streptavidin to a filter support. The myelin basic protein is a substrate for activated MAP kinase. The phosphorylation reaction is carried out for 10 min at 30° C. The extract can then be aspirated through the filter, which retains the phosphorylated myelin basic protein. The filter is washed and counted for ³²P by liquid scintillation counting.

Cell Proliferation Assay

Activation of a G protein coupled receptor may lead to a mitogenic or proliferative response which can be monitored via [³H]-thymidine uptake. When cultured cells are incubated with [³H]-thymidine, the thymidine translocates into the nuclei where it is phosphorylated to thymidine triphosphate. The nucleotide triphosphate is then incorporated into the cellular DNA at a rate that is proportional to the rate of cell growth. Typically, cells are grown in culture for 1-3 days. Cells are forced into quiescence by the removal of serum for 24 hrs. A mitogenic agent is then added to the media. 24 hrs later, the cells are incubated with [³H]-thymidine at specific activities ranging from 1 to 10 μCi/ml for 2-6 hrs. Harvesting procedures may involve trypsinization and trapping of cells by filtration over GF/C filters with or without a prior incubation in TCA to extract soluble thymidine. The filters are processed with scintillant and counted for ³H by liquid scintillation counting. Alternatively, adherent cells are fixed in MeOH or TCA, washed in water, and solubilized in 0.05% deoxycholate/0.1 N NaOH. The soluble extract is transferred to scintillation vials and counted for ³H by liquid scintillation counting.

Methods for Recording Currents in Xenopus oocytes

Female Xenopus laevis (Xenopus-1, Ann Arbor, Mich.) are anesthetized in 0.2% tricain (3-aminobenzoic acid ethyl ester, Sigma Chemical Corp.) and a portion of ovary is removed using aseptic technique (Quick and Lester, 1994). Oocytes are defolliculated using 2 mg/ml collagenase (Worthington Biochemical Corp., Freehold, N.J.) in a solution containing 87.5 mM NaCl, 2 mM KCl, 2 mM MgCl₂ and 5 mM HEPES, pH 7.5. Oocytes may be injected (Nanoject, Drummond Scientific, Broomall, Pa.) with mammalian mRNA. Other oocytes may be injected with a mixture of mammalian mRNA and mRNA encoding the genes for G-protein-activated inward rectifiers (GIRK1 and GIRK4, U.S. Pat. Nos. 5,734,021 and 5,728,535). Genes encoding G-protein inwardly rectifying K⁺ (GIRK) channels 1 and 4 (GIRK1 and GIRK4) were obtained by PCR using the published sequences (Kubo et al., 1993; Dascal et al., 1993; Krapivinsky et al., 1995 and 1995b) to derive appropriate 5′ and 3′ primers. Human heart cDNA was used as template together with the primers

| | |
|---|---|
| 5′-CGCGGATCCATTATGTCTGCACTCCGAAGGAAA | (SEQ ID NO:10) |
| TTTG-3′ and | |
| 5′-CGCGAATTCTTATGTGAAGCGATCAGAGTTCAT | (SEQ ID NO:11) |
| TTTTC-3′ for GIRK1 and | |
| 5′-GCGGGATCCGCTATGGCTGGTGATTCTAGGAAT | (SEQ ID NO:12) |
| G-3′ and | |
| 5′-CCGGAATTCCCCTCACACCGAGCCCCTGG-3′ | (SEQ ID NO:13) |
| for GIRK4. | |

In each primer pair, the upstream primer contained a BamHI site and the downstream primer contained an EcoRI site to facilitate cloning of the PCR product into pcDNA1-Amp (Invitrogen). The transcription template for the mammalian receptor may be similarly obtained. mRNAs are prepared from separate DNA plasmids containing the complete coding regions of the mammalian receptor, GIRK1, and GIRK4. Plasmids are linearized and transcribed using the T7 polymerase (“Message Machine”, Ambion). Alternatively, mRNA may be translated from a template generated by PCR, incorporating a T7 promoter and a poly A⁺ tail. Each oocyte receives 2 ng each of GIRK1 and GIRK4 mRNA in combination with 25 ng of mammalian receptor mRNA. After injection of mRNA, oocytes are incubated at 16° C. on a rotating platform for 3-8 days. Dual electrode voltage clamp (“GeneClamp”, Axon Instruments Inc., Foster City, Calif.) is performed using 3 M KCl-filled glass microelectrodes having resistances of 1-3 Mohms. Unless otherwise specified, oocytes are voltage clamped at a holding potential of −80 mV. During recordings, oocytes are bathed in continuously flowing (2-5 ml/min) medium containing 96 mM NaCl, 2 mM KCl, 2 mM CaCl₂, 2 mM MgCl₂, and 5 mM HEPES, pH 7.5 (“ND96”), or, in the case of oocytes expressing GIRK1 and GIRK4, elevated K⁺ containing 96 mM KCl, 2 mM NaCl, 2 mM CaCl₂, 2 MM MgCl₂, and 5 mM HEPES, pH 7.5 (“hK”). Drugs are applied by switching from a series of gravity fed perfusion lines.

Heterologous expression of GPCRs in Xenopus oocytes has been widely used to determine the identity of signaling pathways activated by agonist stimulation (Gundersen et al., 1983; Takahashi et al., 1987). Activation of the phospholipase C (PLC) pathway is assayed by applying test compound in ND96 solution to oocytes previously injected with mRNA for the mammalian receptor and observing inward currents at a holding potential of −80 mV. The appearance of currents that reverse at −25 mV and display other properties of the Ca⁺⁺-activated Cl⁻ (chloride) channel is indicative of mammalian receptor-activation of PLC and release of IP3 and intracellular Ca⁺⁺. Such activity is exhibited by GPCRs that couple to G_{q}.

Measurement of inwardly rectifying K⁺ (potassium) channel (GIRK) activity is monitored in oocytes that have been co-injected with mRNAs encoding the mammalian receptor, GIRK1, and GIRK4. The two GIRK gene products co-assemble to form a G-protein activated potassium channel known to be activated (i.e., stimulated) by a number of GPCRs that couple to Gᵢ or Gₒ (Kubo et al., 1993; Dascal et al., 1993). Oocytes expressing the mammalian receptor plus the two GIRK subunits are tested for test compound responsivity by measuring K⁺ currents in elevated K⁺ solution (hK). Activation of inwardly rectifying currents that are sensitive to 300 μM Ba⁺⁺ signifies the mammalian receptor coupling to a Gᵢ or Gₒ pathway in the oocytes.

Receptor/G Protein Co-Transfection Studies

A strategy for determining whether MCH1 can couple preferentially to selected G proteins involves co-transfection of MCH1 receptor cDNA into a host cell together with the cDNA for a G protein alpha sub-unit. Examples of G alpha sub-units include members of the Gαi/Gαo class (including Gαt2 and Gαz), the Gαq class, the Gαs class, and the Gα12/13 class. A typical procedure involves transient transfection into a host cell such as COS-7. Other host cells may be used. A key consideration is whether the cell has a downstream effector (a particular adenylate cyclase, phospholipase C, or channel isoform, for example) to support a functional response through the G protein under investigation. G protein beta gamma sub-units native to the cell are presumed to complete the G protein heterotrimer; otherwise specific beta and gamma sub-units may be co-transfected as well. Additionally, any individual or combination of alpha, beta, or gamma subunits may be co-transfected to optimize the functional signal mediated by the receptor.

The receptor/G alpha co-transfected cells are evaluated in a binding assay, in which case the radioligand binding may be enhanced by the presence of the optimal G protein coupling or in a functional assay designed to test the receptor/G protein hypothesis. In one example, the MCH1 receptor may be hypothesized to inhibit cAMP accumulation through coupling with G alpha sub-units of the Gαi/Gαo class. Host cells co-transfected with the MCH1 receptor and appropriate G alpha sub-unit cDNA are stimulated with forskolin +/−MCH1 agonist, as described above in cAMP methods. Intracellular cAMP is extracted for analysis by radioimmunoassay. Other assays may be substituted for cAMP inhibition, including GTPγ³⁵S binding assays and inositol phosphate hydrolysis assays. Host cells transfected with MCH1 minus G alpha or with G alpha minus MCH1 would be tested simultaneously as negative controls. MCH1 receptor expression in transfected cells may be confirmed in radioligand binding studies using membranes from transfected cells. G alpha expression in transfected cells may be confirmed by Western blot analysis of membranes from transfected cells, using antibodies specific for the G protein of interest.

The efficiency of the transient transfection procedure is a critical factor for signal to noise in an inhibitory assay, much more so than in a stimulatory assay. If a positive signal present in all cells (such as forskolin-stimulated cAMP accumulation) is inhibited only in the fraction of cells successfully transfected with receptor and G alpha, the signal to noise ratio will be poor. One method for improving the signal to noise ratio is to create a stably transfected cell line in which 100% of the cells express both the receptor and the G alpha subunit. Another method involves transient co-transfection with a third cDNA for a G protein-coupled receptor which positively regulates the signal which is to be inhibited. If the co-transfected cells simultaneously express the stimulatory receptor, the inhibitory receptor, and a requisite G protein for the inhibitory receptor, then a positive signal may be elevated selectively in transfected cells using a receptor-specific agonist. An example involves co-transfection of COS-7 cells with 5-HT4 receptor, MCH1 receptor, and a G alpha sub-unit. Transfected cells are stimulated with a 5-HT4 agonist +/−MCH1 agonist. Cyclic AMP is expected to be elevated only in the cells also expressing MCH1 and the G alpha subunit of interest, and a MCH1-dependent inhibition may be measured with an improved signal to noise ratio.

It is to be understood that the cell lines described herein are merely illustrative of the methods used to evaluate the binding and function of the mammalian receptors of the present invention, and that other suitable cells may be used in the assays described herein.

Promiscuous Second Messenger Assays

It is possible to coax receptors of different functional classes to signal through a pre-selected pathway through the use of promiscuous G_{α} subunits. For example, by providing a cell based receptor assay system with an exogenously supplied promiscuous G_{α} subunit such as G_{α16} or a chimeric G_{α} subunit such as G_{αzq}, a GPCR which normally might prefer to couple through a specific signaling pathway (e.g. Gₛ, Gₗ, G_{q}, Gₒ, etc.), can be made to couple through the pathway defined by the promiscuous G_{α} subunit and upon agonist activation produce the second messenger associated with that subunit's pathway. In the case of G_{α} and/or G_{αzq} this would involve activation of the G_{q} pathway and production of the second messenger inositol phosphate. Through similar strategies and tools, it is possible to bias receptor signaling through pathways producing other second messengers such as Ca⁺⁺, cAMP, K⁺ currents, etc.

Microphysiometric Assay

Because cellular metabolism is intricately involved in and effected by a broad range of cellular events (including receptor activation of various second messenger pathways), the use of microphysiometric measurements of cell metabolism can in principle provide a generic assay of cellular activity arising from the activation of any receptor regardless of the specifics of the receptor's proximal signaling pathway.

General guidelines for cell preparation and microphysiometric recording have been previously reported (Salon, J. A. and Owicki, J. A., 1996). A typical protocol employing transiently transfected CHO cells is as follows; 24 hours prior to recording, transfected cells are harvested and counted. 3×10⁵ cells are seeded into cell culture capsules (Costar), and allowed to attach to the capsule membrane. 10 hours later (14 hours prior to recording) the cell media is switched to serum free F-12 complete to minimize ill-defined metabolic stimulation caused by assorted serum factors.

On the day of the experiment the cell capsules are transferred to the microphysiometer (Cytosensor, Molecular Devices Corporation, Sunnyvale, Calif.) and allowed to equilibrate in recording media (low buffered RPMI 1640, no bicarbonate, no serum) with 0.1% BSA (essentially fatty acid free), during which a baseline measurement of basal metabolic activity is established. The recording paradigm consists of a 100 μl/min flow rate, with a 2 min pump cycle which includes a 30 sec flow interruption during which the rate measurement is taken. Challenges involve a 1 min 20 sec exposure to a drug just prior to the first post challenge rate measurement being taken, followed by two additional pump cycles for a total of 5 min 20 sec drug exposure. Drug is then washed out and rates allowed to return to basal. Reported extracellular acidification rates are expressed as a percentage increase of the peak response over the baseline rate observed just prior to challenge.

GPCR Ligand Library

Functional assays of new receptors such as MCH1 may include a preliminary test of a small library of compounds containing representative agonists for all known GPCRs as well as other compounds which may be agonists for prospective GPCRs or which may be effectors for targets peripherally involved with GPCRs. The collection used in this study comprises approximately 180 compounds (including small molecules, hormones, preprohormones, peptides, etc.) for more than 45 described classes of GPCRs (serotonin, dopamine, noradrenaline, opioids, etc.) and additionally includes ligands for known or suspected but not necessarily pharmacological characterized or cloned GPCR families (such as MCH).

The diversity of the library can be expanded to include agonist and antagonist compounds specific for GPCR subtypes, combinatorial peptide and/or small molecule libraries, natural product collections, and the like. To facilitate robotic handling, the substances are distributed as either separate or pooled compound concentrates in 96 well plates and stored frozen as ready to use reagent plates.

Localization of mRNA Coding for Human MCH1 Receptors

Development of Probes for MCH1:

To facilitate the production of radiolabeled, antisense RNA probes a fragment of the gene encoding rat MCH1 will be subcloned into a plasmid vector containing RNA polymerase promoter sites. The full length cDNA encoding the rat MCH1 will be digested with Pst 1, (nucleotides 905-1194) and this 289 nucleotide fragment will be cloned into the Pst I site of pGEM 3z, containing both sp6 and T7 RNA polymerase promoter sites. The construct will be sequenced to confirm sequence identity and orientation. To synthesize antisense strands of RNA, this construct will be linearized with Hind III or Eco RI (depending on orientation) and T7 or sp6 RNA polymerase will be used to incorporate radiolabeled nucleotide as described below.

A probe coding for the rat glyceraldehyde 3-phosphate dehydrogenase (GAPDH) gene, a constitutively expressed protein, was used concurrently. GAPDH is expressed at a relatively constant level in most tissue and its detection is used to compare expression levels of the rat MCH1 receptors gene in different regions.

Synthesis of Probes:

MCH1 and GAPDH cDNA sequences preceded by phage polymerase promoter sequences will be used to synthesize radiolabeled riboprobes. Conditions for the synthesis of riboprobes will be: 0.25-1.0 μg linearized DNA plasmid template, 1.5 μl of ATP, GTP, UTP (10 mM each), 3 μl dithiothreitol (0.1 M), 30 units RNAsin RNAse inhibitor, 0.5-1.0 μl (15-20 units/μl) RNA polymerase, 7.0 μl transcription buffer (Promega Corp.), and 12.5 μl α³²P-CTP (specific activity 3,000 Ci/mmol). 0.1 mM CTP (0.02-1.0 μl) will be added to the reactions, and the volume will be adjusted to 35 μl with DEPC-treated water. Labeling reactions will be incubated at 37° C. for 60 min, after which 3 units of RQ1 RNAse-free DNAse (Promega Corp.) will be added to digest the template. Riboprobes will be separated from unincorporated nucleotides using Microspin S-300 columns (Pharmacia Biotech). TCA precipitation and liquid scintillation spectrometry will be used to measure the amount of label incorporated into the probe. A fraction of all riboprobes synthesized will be size-fractionated on 0.25 mm thick 7M urea, 4.5% acrylamide sequencing gels. These gels will be apposed to storage phosphor screens and the resulting autoradiograph scanned using a phoshorimager (Molecular Dynamics, Sunnyvale, Calif.) to confirm that the probes synthesized were full-length and not degraded.

Solution Hybridization/Ribonuclease Protection Assay (RPA):

For solution hybridization 2.0 μg of mRNA isolated from tissues will be used. Negative controls consisted of 30 μg transfer RNA (tRNA) or no tissue blanks. All mRNA samples will be placed in 1.5-ml microfuge tubes and vacuum dried. Hybridization buffer (40 μl of 400 mM NaCl, 20 mM Tris, pH 6.4, 2 mM EDTA, in 80% formamide) containing 0.25-2.0 E⁶counts of each probe will be added to each tube. Samples will be heated at 95° C. for 15 min, after which the temperature will be lowered to 55° C. for hybridization.

After hybridization for 14-18 hr, the RNA/probe mixtures will be digested with RNAse A (Sigma) and RNAse T1 (Life Technologies). A mixture of 2.0 μg RNAse A and 1000 units of RNAse T1 in a buffer containing 330 mM NaCl, 10 mM Tris (pH 8.0) and 5 mM EDTA (400 μl) will be added to each sample and incubated for 90 min at room temperature. After digestion with RNAses, 20 μl of 10% SDS and 50 μg proteinase K will be added to each tube and incubated at 37° C. for 15 min. Samples will be extracted with phenol/chloroform:isoamyl alcohol and precipitated in 2 volumes of ethanol for 1 hr at −70° C. Pellet Paint (Novagen) will be added to each tube (2.0 μg) as a carrier to facilitate precipitation. Following precipitation, samples will be centrifuged, washed with cold 70% ethanol, and vacuum dried. Samples will be dissolved in formamide loading buffer and size-fractionated on a urea/acrylamide sequencing gel (7.0 M urea, 4.5% acrylamide in Tris-borate-EDTA). Gels will be dried and apposed to storage phosphor screens and scanned using a phosphorimager (Molecular Dynamics, Sunnyvale, Calif.).

RT-PCR:

For the detection of RNA encoding human MCH1, RT-PCR was carried out on mRNA extracted from human tissue. Reverse transcription and PCR reactions were carried out in 50 ml volumes using EZrTth DNA polymerase (Perkin Elmer). Primers with the following sequences were used:

| | |
|---|---|
| Forward primer (RA SLC1a /MCH F); | |
| TCA GCT CGG TTG TGG GAG CA | (SEQ ID NO:14) |
| Reverse primer (RA/SLC1a MCH B); | |
| CTT GGA CTT CTT CAC GAC | (SEQ ID NO:15) |

These primers will amplify a 248 base pair fragment from nucleotide 169 to 417.

Each reaction contained 0.1 μg mRNA and 0.3μM of each primer. Concentrations of reagents in each reaction were: 300 μM each of GTP; dATP; dCTP; dTTP; 2.5mM Mn(OAc)2; 50 mM Bicine; 115 mM potassium acetate, 8% glycerol and 5 units EZrTth DNA polymerase. All reagents for PCR (except mRNA and oligonucleotide primers) were obtained from Perkin Elmer. Reactions were carried out under the following conditions: 65° C. 60 min., 94° C. 2 min., (94° C., 1 min., 65° C. 1 min) 35 cycles, 72° C. 10 min. PCR reactions were size fractionated by gel electrophoresis using 10% polyacrylamide. DNA was stained with SYBR Green I (Molecular Probes, Eugene Oreg.) and scanned on a Molecular Dynamics (Sunnyvale Calif.) Storm 860 in blue fluorescence mode at 450 nM.

Positive controls for PCR reactions consisted of amplification of the target sequence from a plasmid construct, as well as reverse transcribing and amplifying a known sequence. Negative controls consisted of mRNA blanks, as well as primer and mRNA blanks. To confirm that the mRNA was not contaminated with genomic DNA, samples were digested with RNAses before reverse transcription. Integrity of RNA was assessed by amplification of mRNA coding for GAPDH.

Receptor Audioradiographic Experiments Localizing the MCH1 Receptor in the Rat CNS

Animals

Male Sprague-Dawley rats (Charles Rivers, Rochester, N.Y.) were euthanized using CO₂ and decapitated and their brains rapidly removed and frozen on crushed dry ice. Coronal sections were cut at 20 μm using a cryostat and thaw-mounted onto gelatin-coated slides then stored at −20° C. until use.

Radioligand Binding Studies

In radioligand binding assays [³H]Compound 10 (specific activity 56 Ci/mmol (NEN, Boston, Mass.) was used at 0.1 nM. Dopamine, prazosin, and phenanthroline were obtained from Sigma (St. Louis, Mo.). Phenylmethylsulfonyl Fluoride (PMSF) was from Calbiochem (La Jolla, Calif.).

In vitro Autoradiography

Tissue sections were allowed to equilibrate to room temperature for one hour. Sections were incubated at 25° C. for 1.5 hours in 50 mM Tris-HCl buffer, pH 7.4, containing 10 mM MgCl₂, 0.16 mM PMSF, 0.3 mM phenanthroline, 0.2% bovine serum albumin (Boehringer Mannheim, Indianapolis, Ind.), 100 μM dopamine, 1 μM prazosin, and 0.01 nM [³H]Compound 10. Nonspecific binding was determined by including 10 μM unlabeled Compound 10 in the incubation buffer. Following incubation the sections were washed twice for 5 minutes each in 4° C. 50 mM Tris-buffer, pH 7.4, then rapidly dipped in ice-cold distilled water to remove the salts. Tissues were dried under a stream of cold air and apposed together with ³H-plastic standard scales, to Hyperfilm-³H (Amersham, Piscataway, N.J.) for 6 weeks. Films were developed using a Kodak developer-D19 and Rapid fixer (Kodak, Rochester, N.Y.). Specific [³H]Compound 10 binding to the MCH1 receptor was interpreted by observation of the remaining optical density on the autoradiogram in the various regions of rat brain in the presence of the appropriate displacers.

Chemical Synthetic Methods

General Methods:

All reactions (except for those done by parallel synthesis reaction arrays) were performed under an Argon atmosphere and the reagents, neat or in appropriate solvents, were transferred to the reaction vessel via syringe and cannula techniques. The parallel synthesis reaction arrays were performed in vials (without an inert atmosphere) using J-KEM heating shakers (Saint Louis, Mo.). Anhydrous solvents were purchased from Aldrich Chemical Company and used as received. The examples described in the patent (1-37) were named using ACD/Name program (version 2.51, Advanced Chemistry Development Inc., Toronto, Ontario, M5H2L3, Canada). Unless otherwise noted, the ¹H and ¹³C NMR spectra were recorded at 300 and 75 MHz (QE Plus) with CDCl₃ as solvent and tetramethylsilane as internal standard. s=singlet; d=doublet; t=triplet; q=quartet; p=pentet; sextet; septet; br=broad; m=multiplet. Elemental analyses were performed by Robertson Microlit Laboratories, Inc. Unless otherwise noted, mass spectra were obtained using low-resolution electrospray (ESMS) and MH+ is reported. Thin-layer chromatography (TLC) was carried out on glass plates precoated with silica gel 60 F254 (0.25 mm, EM Separations Tech.). Preparative thin-layer chromatography was carried out on glass sheets precoated with silica gel GF (2 mm, Analtech). Flash column chromatography was performed on Merck silica gel 60 (230-400 mesh). Melting points (mp) were determined in open capillary tubes on a Mel-Temp apparatus and are uncorrected.

Procedures for the Synthesis of the Dihydropyrimidine Intermediates

5-Methoxycarbonyl-4-Methoxymethyl-1,2,3,6-Tetrahydro-2-oxo-6-(3,4-Difluorophenyl)-Pyrimidine:

To a stirring mixture of methyl 4-methoxyacetoacetate (50.0 g, 0.342 mol), 3,4-difluorobenz-aldehyde (51.4 g, 0.362 mol), and urea (31.6 g, 0.527 mole) in THF (300 mL) at room temperature were added copper(I) oxide (5.06 g, 0.035 mole) and acetic acid (2.05 mL), sequentially, followed by dropwise addition of boron trifluoride diethyl etherate (56.0 mL, 0.442 mole). The mixture was stirred and refluxed for 8 h, whereupon TLC (1/1 EtOAc/hexanes) analysis indicated completion of the reaction. The reaction mixture was cooled and poured into a mixture of ice and sodium bicarbonate (100 g) and the resulting mixture was filtered through Celite. The Celite pad was washed with dichloromethane (400 mL). The organic layer was separated from the filtrate and the aqueous layer was extracted with more dichloromethane (3×300 mL). The combined organic extracts were dried (sodium sulfate) and the solvent evaporated. The crude product was purified by flash column (ethyl acetate/hexanes, 1/1; then ethyl acetate) giving the product as pale yellow foam, which on trituration with hexane became white powder (103 g, 97%). ¹H NMR d 3.48 (s, 3H), 3.65 (s, 3H), 4.65 (s, 2H), 5.39 (s, 1H), 6.60 (br s, 1H, NH), 7.00-7.20 (m, 3H), 7.72 (br s, 1H, NH).

(+)-5-Methoxycarbonyl-4-Methoxymethyl-1,2,3,6-Tetrahydro -2-oxo-6-(3,4-Difluorophenyl)-Pyrimidine:

The racemic intermediate 5-methoxycarbonyl-4-methoxymethyl-1,2,3,6-tetrahydro-2-o xo-6-(3,4-difluorophenyl)pyrimidine was resolved by chiral HPLC [Chiralcel OD 20×250 mm #369-703-30604; lambda 254 nm; hexanes/ethanol 90/10; 85 mg per injection; retention time of the desired enantiomer: 16.94 min., the first enantiomer peak to elute], giving (+)-5-methoxycarbonyl-4-methoxymethyl-1,2,3,6-tetrahydro-2oxo-6-(3,4-difluorophenyl)-pyrimidine (40-42 wt % isolation of the desired enantiomer from the racemate); [α]D=+83.8 (c=0.5, chloroform). The (−)-isomer was also isolated as the later eluting fraction from the chiral chromatography column.

(+)-5-Methoxycarbonyl-4-Methoxymethyl-1,2,3,6-Tetrahydro -2-oxo-6-(3,4-Difluorophenyl)-1-[(4-Nitrophenyloxy) Carbonyl] Pyr imidine:

To a solution of (+)-5-methoxycarbonyl-4-methoxymethyl-1,2,3, 6-tetrahydro-2-oxo-6-(3,4-difluorophenyl)-pyrimidine (1.98 g, 6.34 mmol) in anhydrous THF (20 mL) at −78° C. under argon atmosphere, a solution of lithium hexamethyldisilazide in THF (1M, 18.0 mL, 18.0 mmol) was added over 2-3 min. and the mixture was stirred for 10 min. This solution was added over 6 min., via a cannula, to a stirred solution of 4-nitrophenyl chloroformate (4.47 g, 22.2 mmol) in THF (20 mL) at −78° C. Stirring was continued for 10 min. and the mixture was poured onto ice (50 g) and extracted with chloroform (2×50 mL). The combined extracts were dried (sodium sulfate) and the solvent was evaporated. The residue was purified by flash column chromatography (hexanes/ethyl acetate, 4/1 to 3.5/1) as the eluent. The product was obtained as yellow syrup which upon trituration with hexanes became a white powder (2.40 g, 79%): ¹H NMR d 3.52 (s, 3H), 3.74 (s, 3H), 4.65-4.80 (q, J=16.5 Hz, 2H), 6.32 (s, 1H), 7.10-7.30 (m, 4H), 7.36 (d, J=9 Hz, 2H), 8.27 (d, J=9 Hz, 2H).

Benzyl 3-[(3,4-Difluorophenyl)Methylene]-4-Oxopentanoate:

A solution of benzyl propionylacetate (36.3 g, 176 mmol), 3,4-difluorobenzaldehyde (25.0 g, 176 mmol), piperidine (0.86 mL, 9.0 mmol) and acetic acid (0.49 mL, 9.0 mmol) was refluxed with removal of water using a Dean-Stark apparatus for 5 h. The solvent was removed in vacuo and the residue was dissolved in EtOAc. The reaction mixture was washed with water (100 mL), followed by brine (100 mL) and dried over anhydrous Na₂SO₄. The solvent was evaporated, giving a pale yellow syrup (60.2 g). The product was used in the next step without further purification.

5-(Benzyloxycarbonyl)-1,6-Dihydro-2-Methoxy-4-Ethyl-6-(3,4-Di-Fluorophenyl)pyrimidine:

A suspension of benzyl 3-[(3,4-di-fluorophenyl)methylene]-4-oxopentanoate (16.0 g, 48.0 mmol), O-methylisourea hydrogen sulfate (16.7 g, 97.0 mmol) and NaHCO3 (16.3 g, 130 mmol) in DMF (190 mL) was stirred at 70° C. for 20 h. After cooling to room temperature, the mixture was filtered and the filtrate was diluted with EtOAc (300 mL) and then washed with water (4×100 mL), brine (200 mL) and dried over Na₂SO₄. After removal of solvent, the residue was purified by column chromatography (EtOAc/Hexane, 1/9 to 3/7), giving the title compound as a colorless oil (10.6 g, 58%). The NMR analysis showed it to be a mixture of amine/imine tautomers and was used as is in the next step.

5-(Benzyloxycarbonyl)-4-Ethyl-1,6-Dihydro-2-Methoxy-6-(3,4-Di-Fluorophenyl)-1-[(4-Nitrophenyloxy)Carbonyl]Pyrimidine:

To a stirring solution of 5-(benzyloxycarbonyl)-1,6-dihydro-2-methoxy-4-ethyl-6-(3,4-difluorophenyl)pyrimidine (17.0 g, 44.0 mmol) and 4-dimethylaminopyridine (7.00 g, 57.3 mmol) in CH₂Cl₂ (200 mL) was added 4-nitrophenyl chloroformate as a powder (11.5 g, 57.1 mmol) at room temperature. The reaction mixture was stirred for 12 h and then the solvent was removed in vacuo. The residue was purified by chromatography (EtOAc/Hexane, 1/9 to 3/7), giving 5-(benzyloxycarbonyl)-4-ethyl-1,6-dihydro-2-methoxy-6-(3,4-difluorophenyl)-1-[(4-nitrophenyloxy)carb onyl]pyr-imidine as a colorless viscous oil (12.6 g, 50%). ¹H NMR d 1.24 (t, J=7.2 Hz, 3H), 2.81-2.98 (m, 3H), 3.97 (s, 3H), 5.14 (ABq, A=5.08, B=5.20, J=12.3 Hz, 2H), 6.28 (s, 3H), 7.03-7.29 (m, 8H), 7.35 (d, J=9.2 Hz, 2H) 8.26 (d, J=9.2 Hz, 2H).

5-(Benzyloxycarbonyl)-4-Ethyl-1,6-Dihydro-1-{N-[1-Phenyl) Ethyl]}-Carboxamido-2-Methoxy-6-(3,4-Difluorophenyl) Pyrimidine:

To a stirred mixture of 5-(benzyloxycarbonyl)-4-ethyl-1,6-dihydro-2-methoxy-6-(3,4-difluorophenyl)-1-[(4-nitrophenyloxy) carb onyl]pyr-imidine (12.6 g, 22.9 mmol) in THF (150 mL) was added a solution of R-(+)-α-methyl benzylamine (3.53 mL, 27.1 mmol) at room temperature. The stirring was continued for 12 h and the solvent was removed in vacuo. The yellow residue was dissolved in chloroform (200 mL) and was washed with 10% K₂CO₃ solution (2×30 mL). The organic layer was dried over Na₂SO₄, filtered and solvent was removed in vacuo. The resulting mixture of diastereomers was separated by column chromatography (petroleum ether/ether, 9/1 to 4/1). The first major product to elute was (+)-5-(benzyloxycarbonyl)-4-ethyl-1,6-dihydro-1-{N-[1-phenyl)-ethyl]}carboxamido-2-methoxy-6-(3,4-difluorophenyl)pyrim idine. Colorless oil; Rf=0.31 (petroleum ether/ether, 4/1); yield: 3.8 g (31%); [α]_{D}+267.05 (c=0.76, CHCl₃);

¹H NMR d 1.22 (t, J=7.5 Hz, 3H), 1.52 (d, J=6.9 Hz, 3H), 2.88 (q, J=6.0 Hz, 2H), 3.99 (s, 3H), 4.99 (m, 1H), 5.09 (ABq, A=5.00, B=5.19, J=12.6 Hz, 2H), 6.66 (s, 1H), 6.99-7.36 (m, 13H). The second major product to elute was (−)-5-(benzyloxycarbonyl)-4-ethyl-1,6-dihydro-1-{N-[2-phenyl)ethyl]}carboxamido-2-methoxy-6-(3,4-difluoroph enyl)pyr-imidine. Colorless oil; Rf=0.22 (petroleum ether/ether, 4/1); yield: 3.20 g (26%); [α]_{D}=−146.89 (c=0.38, CHCl₃); ¹H NMR δ 1.22 (t, J=7.2 Hz, 3H), 1.49 (d, J=6.6 Hz, 3H),2.88 (q, J=6.0 Hz, 2H), 3.94 (s, 3H), 5.03 (m, 1H), 5.11 (ABq, A=5.02, B=5.19, J=12.6 Hz, 2H), 6.68 (s, 1H), 6.91-7.34 (m, 13H).

(+)-5-(Benzyloxycarbonyl)-1,6-Dihydro-2-Methoxy-4-Ethyl-6-(3,4-Di-Fluorophenyl)Pyrimidine:

To a stirred solution of (+)-5-(benz-yloxycarbonyl)-4-ethyl-1,6-dihydro-1-{N-[2-phenyl)ethyl] }carbox-amido-2-methoxy-6-(3,4-difluorophenyl)pyrimidine (1.00 g, 1.83 mmol) in toluene (10 mL) was added 1,8-diazabicyclo[5,4,0]-undec-7-ene (0.120 mL, 0.810 mmol) at room temperature and the resulting solution was heated at reflux temperature for 5 h and then stirred for 12 h at room temperature. The solvent was evaporated and the residue was purified by flash column (EtOAc/Hexanes, 1/3), giving (+)-5-(benzyloxycarbonyl)-1,6-dihydro -2-methoxy-4-ethyl-6-(3,4-difluorophenyl)pyrimidine (0.560 g, 77%).

(+)-5-(Benzyloxycarbonyl)-4-Ethyl-1,6-Dihydro-2-Methoxy-6-(3,4-Di-Fluorophenyl)-1-[(4-Nitrophenyloxy)Carbonyl]Pyrimidine:

To a stirring solution of (+)-5-(benzyloxycarbonyl)-1,6-dihydro-2-methoxy-4-ethyl-6-(3,4-difluorophen-yl)pyrimidine (17.0 g, 44.0 mmol) and 4-dimethylaminopyridine (6.99 g, 57.3 mmol) in CH₂Cl₂ (200 mL) was added 4-nitrophenyl chloroformate (11.6 g, 57.3 mmol) at room temperature. The reaction mixture was stirred for 12 h and then the solvent was removed in vacuo. The residue was purified by chromatography (EtOAc/Hexane, 1/9 to 3/7), giving (+)-5-(benzyloxycarbonyl)-4-ethyl-1,6-dihydro-2-methoxy-6-(3,4-difluorophenyl)-1-[(4-nitrophenyloxy) carbonyl]pyrimidine as a viscous colorless oil (19.3 g, 76%).

5-Methylbenzfuroxan:

4-Methyl-2-nitroaniline (100 g, 0.650 mol) was suspended in saturated methanolic sodium hydroxide solution (1.50 L). This suspension was cooled (5° C.) and aqueous sodium hypochlorite until the red color disappeared. The resulting fluffy yellow precipitate was filtered, washed with cold water and recrystallized from ethanol, giving 5-methylbenzfuroxan (88.2 g, 89% yield) as a pale yellow solid: ¹H NMR d 2.39 (s, 3H), 6.90-7.40 (br m. 3H).

5-Methylbenzofurazan:

To 5-Methylbenzfuroxan (88.2 g, 0.590 mol) in refluxing EtOH (75 mL) was added dropwise P(OEt)₃ (150 mL). Heating was continued at reflux temperature for 1 h. The solvent was removed in vacuo and the residue was shaken with water (200 mL) and allowed to stand overnight at (0-5° C.). The resulting brown solid was filtered, washed with water. The crude product was purified by flash chromatography, giving 5-methylbenzofurazan (70.0 g, 87%) as white needles; ¹H NMR δ 2.41 (s, 1H), 7.19 (dd, J=9.3, 1.1 Hz, 1H), 7.48 (d, J=1.1 Hz, 1H), 7.66 (d, J=9.3 Hz, 1H).

5-Dibromomethylbenzofurazan:

An anhydrous solution of 5-methylbenzofurazan (70.0 g, 0.520 mol), N-bromosuccinamide (325 g), and benzoyl peroxide (0.50 g) in carbon tetrachloride (1.5 L) was heated at reflux temperature with stirring for 30 h. The reaction mixture was washed with water (2×500 mL), dried (NaSO₄), and the solvent was removed in vacuo. The residue was chromatograghed (EtOAc/hexane, 1/150), giving 122 g (80%) of the title compound as a white solid: ¹H NMR d 6.69 (s, 1H), 7.69 (d, J=9.6 Hz, 1H), 7.77 (s, 1H), 7.89 (d, J=9.6 Hz, 1H).

5-Formylbenzofurazan:

AgNO₃ (163 g) in 2 L of water was added to a refluxing mixture of dibromomethylbenzofurazan (122 g, 418 mmol) in EtOH (1 L). Heating at reflux temperature was continued for 2 h. The mixture was cooled, the precipitated AgBr was removed by filtration through Celite, and the solvent was concentrated. The resulting solution was extracted with toluene (10×100 mL), dried over magnesium sulfate, and the solvent was removed in vacuo. The residue was chromatograghed (EtOAc/hexane, 1/125), giving the title aldehyde (48.2 g, 78%) as a white solid: ¹H NMR δ 7.92 (m, 2H), 8.39 (s, 1H), 10.10 (s, 1H).

Methyl 2-{(Benzofuran-5-yl)Methylene}-3-Oxobutyrate:

A mixture of 5-formylbenzofurazan (0.60 g, 4.1 mmol), methyl acetoacetate (0.52 g, 4.5 mmol), piperidine (0.019 g, 0.23 mmol), and acetic acid (0.014 g, 0.23 mmol) in benzene (30 mL) was heated at reflux temperature (equipped with a Dean-Stark trap) for 8 h. Benzene was evaporated in vacuo, the residue was dissolved in ethyl acetate (80 mL) and washed with brine (50 mL), saturated potassium bisulfate solution (50 mL), and saturated sodium bicarbonate solution. The ethyl acetate solution was dried over magnesium sulfate, the solvent removed under reduced pressure and the residue was purified by column chromatography (EtOAc/hexane, 1/9 to 3/20). The desired product was obtained as oil (0.98 g, 98%) and was used in the next step without any further characterization.

6-(Benzofurazan-5-yl)-1,6-Dihydro-2-Methoxy-5-Methoxycar Bonyl-4-Methylpyrimidine:

A mixture of methyl 2-{(benzofuran-5-yl)-methylene}-3-oxobutyrate (1.02 g, 4.10 mmol), O-methylisourea hydrogen sulfate (1.06 g, 6.20 mmol), and NaHCO₃ (1.30 g, 16.4 mmol) in DMF (15 mL) was stirred and heated at 70° C. for 16 h. The mixture was cooled, diluted with EtOAc (50 mL) and washed with water (5×50 mL), brine (50 mL) and dried over magnesium sulfate. The solvent was evaporated and the crude product was purified by flash chromatography (EtOAc/hexane, 1/9 to 1/5), giving the desired product as an oil (0.520 g, 43%): ¹HNMR δ 2.38 and 2.42 (2 s, 3H), 3.60 and 3.66 (2 s, 3H), 3.74 and 3.82 (2 s, 3H), 5.53 and 5.68 (2 s, 1H), 6.31 and 6.32 (br s, 1H), 7.0-7.8 (m, 3H).

6-(Benzofurazan-5-yl)-1,6-Dihydro-2-Methoxy-5-Methoxycar Bonyl-4-Methyl-1-[(4-Nitrophenyloxy)Carbonyl]Pyrimidine:

To a solution of 6-(benzofuran-5-yl)-1,6-dihydro-2-methoxy -5-methoxycarbonyl-4-methylpyrimidine (0.485 g, 1.6 mmol) and 4-dimethylaminopyridine (0.200 g, 1.64 mmol) in CH₂Cl₂ (20 mL) at 0-5° C. was added 4-nitrophenyl chloroformate (0.307 g, 1.52 mmol). The mixture was then allowed to warm to room temperature. After 12 h, the solvent was evaporated and the residue was purified by flash chromatography (EtOAc/hexane, 1/9 to 3/20), giving the desired product as white crystals (0.665 g, 89%); mp 180-183° C.; ¹H NMR δ 2.54 (s, 3H), 3.75 (s, 3H), 3.98 (s, 3H), 6.37 (s, 1H), 7.40 (d, J=9.3 Hz, 2H), 7.52 (d, J=9.0 Hz, 1H), 7.68 (s, 1H), 7.84 (d, J=9.0 Hz, 1H), 8.32 (d, J=9.3 Hz, 2H).

(+) and (−)-6-(Benzofurazan-5-yl)-1,6-Dihydro-2-Methoxy-5-Methoxycarbonyl-1-[N-(S)-1-(1-Phenylethyl)]-4-Methylpyri Midine:

A solution of 6-(benzofurazan-5-yl)-1,6-dihydro-2-methoxy-5-methoxycarbonyl-4-methyl -1-(4-nitrophenoxy) carbonylpyrimidine (800 mg, 1.71 mmol) and (S)-(−)-a-methylbenzylamine (269 mg, 2.22 mmol) in THF (50 mL) was stirred at room temperature for 12 h. The THF was removed in vacuo and the residue was dissolved in EtOAc (100 mL), washed by 10% aqueous K₂CO₃ solution (3×50 mL), brine (50 mL) and dried (Na₂SO₄). After removal of the solvent, the residue was purified by chromatography (EtOAc/hexane, 1/20 to 3/20), separating the two diastereomers. The isomers of 6-(benzofurazan-5-yl)-1,6-dihydro-2-methoxy-5-methoxycarbonyl-1-[N-(S)-1-(1-phenylethyl)]-4-methylpyrimidine were obtained as colorless oils. 1st Isomer (367 mg, 47.7%): [α]_{D}=+278 (c=0.50, CHCl₃); ¹H NMR δ 1.54 (d, J=6.9 Hz, 3H), 2.45 (s, 3H), 3.68 (s, 3H), 3.99 (s, 3H), 5.02 (quintet, J=6.9 Hz, 1H), 6.71 (s, 1H), 6.89 (d, J=6.6 Hz, 1H), 7.2-7.9 (m, 8H). 2nd Isomer (205 mg, 26.6%): [α]_{D}=−81 (c=0.43, CHCl₃); ¹H NMR δ 1.52 (d, J=6.6 Hz, 3H), 2.48 (s, 3H), 3.71 (s, 3H), 3.96 (s, 3H), 5.00 (quintet, J=6.6 Hz, 1H), 6.74 (s, 1H), 6.90 (d, J=6.5 Hz, 1H), 7.2-7.9 (m, 8H).

6-(Benzofurazan-5-yl)-1,6-Dihydro-2-Methoxy-5-Methoxycar Bonyl-4-Methylpyrimidne:

A solution of the 1st isomer of 6-(benzofura-zan-5-yl)-1,6-dihydro-2-methoxy-5-methoxycarbon-yl-1-[N-(S)-1-(1-phenylethyl)]-4-methylp yrimidine (960 mg, 2.14 mmol) and 1,8-diazabicyclo[5,4,0]undec-7-ene (107 mg, 0.705 mmol) in toluene (50 mL) was stirred at 100° C. for 5 h. After cooling to room temperature, toluene was removed in vacuo and the residue was purified by chromatography (EtOAc/hexane, 1/9 to 3/7). 6-(Benzofurazan-5-yl)-1,6-dihydro-2-methoxy-5-methoxycarbonyl-4-methylpyrimidine was obtained as a colorless oil (635 mg, 98.3%). ¹H NMR δ 2.38 (s, 3H), 3.66 (s, 3H), 3.74 (s, 3H), 5.68 (s, 1H), 6.32 (br s, 1H), 7.0-7.8 (m, 3H).

6-(Benzofurazan-5-yl)-1,6-Dihydro-2-Methoxy-5-Methoxycar bonyl-4-Methyl-1-(4-Nitrophenoxy)Carbonylpyrimidine:

To a solution of 6-(benzofuran-5-yl)-1,6-dihydro-2-methoxy-5-methoxycarbonyl-4-methylpyrimidine (0.485 g, 1.60 mmol) and 4-dimethylamino-pyridine (0.200 g, 1.60 mmol) in CH₂Cl₂ (20 mL), at 0-5° C., was added 4-nitrophenyl chloroformate (0.307 g, 1.52 mmol). After addition, the mixture was allowed to warm to room temperature. After 12 hours, the solvent was evaporated and the residue was purified by flash column chromatography (EtOAc/hexane, 1/9 to 3/20), giving the desired product as white crystals (0.665 g, 89%): mp 180-183° C.; ¹H NMR δ 2.54 (s, 3H), 3.75 (s, 3H), 3.98 (s, 3H), 6.37 (s, 1H), 7.40 (d, J=9.3 Hz, 2H), 7.52 (d, J=9.0 Hz, 1H), 7.68 (s, 1H), 7.84 (d, J=9.0 Hz, 1H), 8.32 (d, J=9.3 Hz, 2H); [α]_{D}=+266 (c=2.70, CH₂Cl₂).

Methyl 2-{(3,4-Difluorophenyl)Methylene}-3-Oxobutyrate:

A mixture of 3,4-difluorobenzaldehyde (14.2 g, 0.100 mol), methyl acetoacetate (12.2 g, 0.105 mol), piperidine (0.430 g, 5 mmol), and acetic acid (0.30 g , 5 mmol) in benzene (150 mL) was stirred and heated at reflux temperature (equipped with a Dean-Stark trap) for 8 h. The benzene was evaporated and the residue was dissolved in ethyl acetate (200 mL). The resulting solution was washed with brine (50 mL), saturated potassium bisulfate solution (50 mL), and saturated sodium bicarbonate solution. The ethyl acetate solution was dried over magnesium sulfate and the solvent was removed under reduced pressure. The residue was purified by column chromatography (EtOAc/hexane, 1/9 to 3/20), giving the desired product as a yellow oil (9.80 g, 41%) which was used in the subsequent step without any further characterization.

6-(3,4-Difluorophenyl)-1,6-Dihydro-2-Methoxy-5-Methoxyca rbonyl-4-Methylpyrimidine:

A mixture of methyl 2-{(3,4-difluorophenyl)-methylene}-3-oxobutyrate (8.80 g, 36.3 mmol), 0-methylisourea hydrogen sulfate (9.40 g, 546 mmol), and NaHCO3 (12.3 g, 146 mol) in DMF (30 mL) was heated at 70° C. with stirring for 16 h. The mixture was cooled, diluted with EtOAc (300 mL) and washed with water (5×300 mL), brine (300 mL), and dried over magnesium sulfate. The solvent was evaporated and the crude product was purified by flash chromatography (EtOAc/hexane, 1/9 to 3/7) as the gradient eluent, giving the desired product as an oil (3.82 g, 35%).

6-(3,4-Difluorophenyl)-1,6-Dihydro-2-Methoxy-5-Methoxyca rbonyl-4-Methyl-1-[(4-Nitrophenyloxy)Carbonyl]Pyrimidine:

4-Nitrophenyl chloroformate (1.82 g, 9.04 mmol) was added to a solution of 6-(3,4-difluorophenyl)-1,6-dihydro-2-methoxy-5-methoxycarbonyl-4-methylpyrimidine (2.82 g, 9.46 mmol) and 4-dimethylaminopyridine (1.16 g, 9.52 mmol) in CH₂Cl₂ (50 mL), at 0-5° C. and the mixture was then allowed to warm to room temperature. After 12 h, the solvent was evaporated and the residue was purified by flash chromatography (EtOAc/hexane, 1/9 to 3/20), giving the desired product as white crystals (3.72, 85%): mp 172-174° C.

6-(3,4-Difluorophenyl)-1,2,3,6-Tetrahydro-2-oxo-5-Methox ycarbon-yl-4-Methyl-1-(4-Nitrophenoxy) Carbonylpyrimidine: Aqueous 6 N hydrochloric acid (10 mL) was added to a stirring solution of 6-(3,4-difluorophenyl)-1,6-dihydro-2-methoxy-5-methoxycarbonyl-4-methyl-1 (4-nitrophenoxy) carbonylpyrimidine (10.0 g) in THF (200 mL) at room temperature. The stirring was continued for 3 h. The solvent was evaporated and the residue was dried under vacuum, giving the desired product as a white powder (9.70 g, 100%): mp 185-186° C.

(+)-1-(3-Bromo-Propylcarbamoyl)-6-(3,4-Difluorophenyl)-4 -Methyl-2-oxo-1,6-Dihydro-Pyrimidine-5-Carboxylic Acid Methyl Ester:

A solution of 10% aqueous HCl (5 mL) was added to a stirring solution of (+)-6-(3,4-difluorophenyl)-1,6-dihydro-2-methoxy-5-methoxycarbonyl-4-methyl-1-[(4-nitrophenyloxy)-carbonyl]pyrim-idine (4.10 g, 9.10 mmol) in THF (20 mL) at room temperature and the resulting solution was stirred overnight. The THF was removed in vacuo and the resulting residue was extracted with EtOAc (3×20 mL), washed with brine (10 mL) and then dried over Na₂SO₄. The solvent was removed in vacuo, giving (+)-6-(3,4-di-fluorophenyl)-1,6-dihydro-2-oxo -5-methoxycarbonyl -4-methyl -1-[(4-nitrophenyloxy)carbonyl]pyrimidine as a viscous oil (3.8 g, 8.5 mmol). The oil was dissolved in THF (20 mL) and 3-bromo-propylamine hydrobromide (2.33 g, 10.8 mmol) and NaHCO₃ (1.81 g, 21.5 mmol) were added. The resulting suspension was stirred at room temperature overnight. The THF was removed in vacuo and the resulting residue was dissolved in water (10 mL) and then extracted with EtOAc (3×20 mL). The EtOAc extracts were combined, dried over Na₂SO₄, filtered and the solvent was removed, giving (+)-1-(3-bromo-propylcarbamoyl)-6-(3,4-difluorophenyl)-4-methyl-2-oxo-1,6-dihydropyrimidine-5-carboxylic acid methyl ester (3.28 g, 83%): ¹H NMR δ 2.05-2.15 (m, 2H), 2.43 (s, 3H), 3.40-3.56 (m, 4H), 3.72 (s, 3H), 6.69 (s, 1H), 7.08-7.27 (m, 3H), 7.57 (br s, 1H), 8.84 (br t, 1H). Anal. Calcd for C₁₇H₁₈N₃O₄ F₂Br: C, 45.76; H, 4.07; N, 9.42. Found: C, 45.70; H, 3.99; N, 9.16.

3-{(3,4,5-Trifluorophenyl)Methylene}-2,4-Pentanedione:

A stirring mixture of 3,4,5-trifluorobenzaldehyde (4.20 g, 26.2 mmol), 2,4-pentanedione (2.62 g, 26.2 mmol), piperidine (0.430 g, 5.00 mmol) in benzene (150 mL) was heated at reflux temperature (equipped with a Dean-Stark trap) for 8 h. The benzene was evaporated and the yellow oily residue 2-{(3,4,5-trifluorophenyl)methylene}-2,4-pentanedione, was used in the next step without further purification.

6-(3,4,5-Trifluorophenyl)-1,6-Dihydro-2-Methoxy-5-Acetyl -4-Methylpyrimidine:

A mixture of 2-{(3,4,5-trifluorophenyl)-methylene}-2,4-pentanedione (26.2 mmol), O-methylisourea hydrogen sulfate (3.22 g, 39.3 mmol), and NaHCO₃ (6.6 g, 78.6 mmol) in EtOH (400 mL) was stirred and heated at 95-100° C. for 6 h. The mixture was filtered and the solid residue was washed with ethanol (100 mL). The solvent was evaporated from the combined filtrates and the crude product was purified by flash column chromatography (EtOAc/hexane, 1/9 to 1/4), giving the desired product as an oil (2.80 g, 36%).

6-(3,4,5-Trifluorophenyl)-1,6-Dihydro-2-Methoxy-5-Acetyl -4-Meth-yl-1-[(4-Nitrophenyloxy)Carbonyl]Pyrimidine:

4-Nitrophenyl chloroformate (1.89 g, 9.38 mmol) was added to a solution of 6-(3,4,5-trifluorophenyl)-1,6-dihydro-2-methoxy-5-acetyl-4-meth-ylpyrimidine (2.80 g, 9.38 mmol) and pyridine (10 mL) in CH₂Cl₂ (200 mL) at 0-5° C., and the resulting mixture was allowed to warm to room temperature. After 12 h, the solvent was evaporated and the residue was purified by flash chromatography (dichloro-methane/EtOAc, 1/9 to 3/20), giving the desired product as a white powder (4.00 g, 92%).

6-(3,4,5-Trifluorophenyl)-1,2,3,6-Tetrahydro-2-oxo-5-Ace tyl-4-Methyl-1-[(4-Nitrophenyloxy)Carbonyl]Pyrimidine:

A solution of 6 N aqueous HCl (4 mL) was added to a stirring solution of 6-(3,4,5-trifluorophenyl)-1,6-dihydro-2-methoxy -5-acetyl-4-methyl-1-[(4-nitrophenyloxy)carbonyl]pyrimidine (4.00 g, 8.63 mmol) in THF (100 mL) at 0-5° C., and the mixture was allowed to warm to room temperature. After 2 h, solvent was evaporated and the product dried under vacuum. The product was obtained as a pure single component and used in the next step without any further purification (3.88 g, 100%).

Procedures for the Synthesis of the Piperidine Intermediates

(reference for the general procedure for Pd coupling of vinyl triflate and boronic acids or tributyl tin reagents: See, Wuston, Wise Synthesis (1991), 993)

Piperidine Side Chain Intermediates

Tert-Butyl 4-{[(Trifluoromethyl)Sulfonyl]oxy}-1,2,3,6-Tetrahydro-1-Pyridinecarboxylate:

n-Butyl lithium (17.6 mL, 44.2 mmol, 2.5 M in hexanes) was added to a solution of diisopropyl amine (96.2 mL, 44.2 mmol) in 40 mL of dry THF at 0° C. and stirred for 20 minutes. The reaction mixture was cooled to −78° C. and tert-butyl 4-oxo-1-piperidinecarboxylate (Aldrich Chemical Company, 40.0 mmol) in THF (40 mL) was added dropwise to the reaction mixture and stirred for 30 minutes. Tf₂NPh (42.0 mmol, 15.0 g) in THF (40 mL) was added dropwise to the reaction mixture and stirred at 0° C. overnight. The reaction mixture was concentrated in vacuo, re-dissolved in hexanes:EtOAc (9:1), passed through a plug of alumina and the alumina plug was washed with hexanes:EtOAc (9:1). The combined extracts were concentrated to yield 16.5 g of the desired product that was contaminated with some starting Tf₂NPh.

¹H NMR (400 MHz, CDCl₃) δ 5.77 (s, 1H), 4.05 (dm, 2H, J=3.0 Hz), 3.63 (t, 2H, J=5.7 Hz), 2.45 (m, 2H), 1.47 (s, 9H).

Tert-Butyl 4-[3-(Amino)Phenyl]-1,2,3,6-Tetrahydro-1-Pyridinecarboxylate:

A mixture of 2 M aqueous Na₂CO₃ solution (4.2 mL), tert-butyl 4-{[(trifluoromethyl)sulfonyl]oxy}-1,2,3,6-tetrahydro-1-pyridine-carboxylate (0.500 g, 1.51 mmol), 3-aminophenylboronic acid hemisulfate (0.393 g, 2.11 mmol), lithium chloride (0.191 g, 4.50 mmol) and tetrakis-triphenylphosphine palladium (0) (0.080 g, 0.075 mmol) in dimethoxyethane (5 mL) was heated at reflux temperature for 3 hours, under an inert atmosphere (an initial degassing of the mixture is recommended to prevent the formation of triphenylphosphine oxide). The organic layer of the cooled reaction mixture was separated and the aqueous layer was washed with ethyl acetate (3×). The combined organic extracts were dried and concentrated in vacuo. The crude product was chromatograghed (silica, hexanes:EtOAc:dichloromethane (6:1:1) with 1% added isopropylamine to protect the BOC group from hydrolysis) to give 0.330 g of the desired product in 81% yield:

¹H NMR (400 MHz, CDCl₃) δ 7.12 (t, 1H, J=7.60 Hz), 6.78 (d, 1H, J=8.4 Hz), 6.69 (t, 1H, J=2.0 Hz), 6.59 (dd, 1H, J=2.2, 8.0 Hz), 6.01 (m, 1H), 4.10-4.01 (d, 2H, J=2.40 Hz), 3.61 (t, 2H, J=5.6 Hz), 2.52-2.46 (m, 2H), 1.49 (s, 9H); ESMS m/e: 275.2 (M+H)⁺.

Anal. Calc. for C₁₆H₂₄N₂O₂: C, 70.04; H, 8.08; N, 10.21.

Found: C, 69.78; H, 7.80; N, 9.92.

Tert-Butyl 4-[3-(Amino)Phenyl]-1-Piperidinecarboxylate

A mixture of 3.10 g of tert-butyl 4-(3-aminophenyl)-1,2,3,6-tetrahydropyridine-1-carboxylate (11.3 mmol) and 1.0 g of 10% Pd/C in 200 mL of ethanol was hydrogenated at room temperature using the balloon method for 2 days. The reaction mixture was filtered and washed with ethanol. The combined ethanol extracts were concentrated in vacuo and the residue was chromatographed on silica (dichloromethane: methanol 95:5 with 1% isopropylamine added to protect the BOC group from hydrolysis) to give 2.63 g of the desired product (84%).

Tert-Butyl 4-(3-Nitrophenyl)-3,6-Dihydro-1(2H)-Pyridinecarboxylate

¹H NMR (400 MHz, CHCl₃) δ 8.23 (s, 1H), 8.11 (d, 1H, J=8.0 Hz), 7.69 (d, 1H, J=8.0 Hz), 7.51 (t, 1H, J=8.0 Hz), 6.20 (m, 1H), 4.17-4.08 (m, 2H), 3.67 (t, 2H, J=5.6 Hz), 2.61-2.52 (m, 2H), 1.50 (s, 9H); ESMS m/e: 249.1 (M+H−C₄H₈)⁺.

1,2,3,6-Tetrahydro-4-(3-Nitrophenyl)Pyridine:

Into a stirred solution of 5.00 g (16.0 mmol) of tert-butyl 1,2,3,6-tetrahydro-4-(3-nitrophenyl)pyridine-1-carboxylate in 100 ml of 1,4-dioxane at 0° C. was bubbled HCl gas for 10 minutes. The reaction mixture was allowed to warm to room temperature and the bubbling of the HCl gas was continued for an additional 1 hour. The solvent was removed in vacuo, the residue was dissolved in 50 mL of water and was neutralized by the addition of KOH pellets. The aqueous solution was extracted with 3×80 mL of dichloromethane and the combined organic extracts were dried (MgSO₄), filtered and concentrated in vacuo. The residue was purified by column chromatography (silica, 9:1, dichloromethane methanol+1% isopropyl amine) to afford 2.85 g (87.5% yield) of the desired product: ¹H NMR (400 MHz, CDCl₃) δ 8.24 (s, 1H), 8.09 (d, 1H, J=8.4 Hz), 7.71 (d, 1H, J=8.0 Hz), 7.49 (t, 1H, J=8.0 Hz), 6.35-6.25 (m, 1H), 3.58 (apparent q, 2H, J=3.0 Hz), 3.14 (t, 2H, J=5.6 Hz), 2.54-2.46 (m, 2H).

Tert-Butyl 3-(4-(3-Nitrophenyl)-3,6-Dihydro-1(2H)-Pyridinyl)Propylcarbamate:

A mixture of 2.80 g (14.0 mmol) of 1,2,3,6-tetrahydro-4-(3-nitrophenyl)pyridine, 3.60 g (15.0 mmol) of tert-butyl N-(3-bromopropyl)carbamate, 11.6 g (84.0 mmol) of K₂CO3, 14.6 mL (84.0 mmol) of diisopropylethylamine and 0.78 g (2.00 mmol) of tetrabutylammonium iodide in 250 mL of 1,4-dioxane was heated at reflux temperature for 14 hours. The reaction mixture was filtered and the filtrate was dried (MgSO₄), concentrated in vacuo and the residue was purified by column chromatography (silica, 9:1, dichloromethane:methanol+1% isopropyl amine) to afford 4.35 g (85.7% yield) of the desired product: ¹H NMR (400 MHz, CDCl₃) δ 8.24 (t, 1H, J=1.9 Hz), 8.09 (dd, 1H, J=1.9, 8.0 Hz), 7.70 (apparent d, 1H, J=8.0 Hz), 7.49 (t, 1H, J=8.0 Hz), 6.23 (m, 1H), 3.29-3.18 (m, 4H), 2.75 (t, 2H, J=5.6 Hz), 2.64-2.54 (m, 4H), 1.82-1.70 (m, 2H), 1.44 (s, 9H); ESMS m/e: 362.2 (M+H)⁺.

3-(4-(3-Nitrophenyl)-3,6-Dihydro-1(2H)-Pyridinyl)-1-Propanamine:

Into a stirred solution of 4.35 (12.0 mmol) of tert-butyl 3-(4-(3-nitrophenyl)-3,6-dihydro-1(2H)-pyridinyl)propylcarbamate in 100 ml of 1,4-dioxane at 0° C. was bubbled HCl gas for 10 minutes. The reaction mixture was allowed to warm to room temperature and the bubbling was continued for an additional 1 hour. The solvent was removed in vacuo, the residue was dissolved in 50 mL of water and was neutralized by the addition of KOH pellets. The aqueous solution was extracted with 3×80 mL of dichloromethane, the combined organic extracts were dried (MgSO₄), filtered and concentrated in vacuo. The residue was purified by column chromatography (silica, 9:1, dichloromethane:methanol+1% isopropyl amine) to afford 3.05 g (97.0% yield) of the desired product: ¹H NMR (400 MHz, CDCl₃) δ 8.24 (t, 1H, J=1.8 Hz), 8.09 (dd, 1H, J=1.8, 8.2 Hz), 7.69 (dd, 1H, J=1.8, 8.2 Hz), 7.48 (t, 1H, J=8.2 Hz), 6.24 (m, 1H), 3.21 (d, 2H, J=3.6 Hz), 2.84 (t, 2H, J=6.6 Hz), 2.75 (t, 2H, J=5.8 Hz), 2.64-2.54 (m, 4H), 1.76 (m, 2H); ESMS m/e: 262.2 (M+H)⁺; Anal. Calc. for C₁₄H₁₉N₃O₂ (0.06 CHCl₃): C, 62.90; H, 7.16; N, 15.65. Found: C, 63.20; H, 7.16; N, 15.65.

Methyl (4S)-3-[({3-[4-(3-Aminophenyl)-1-Piperidinyl]Propyl}Amino)Carbonyl]-4-(3,4-Difluorophenyl)-6-(Methoxymethyl)-2-oxo-1,2,3,4-Tetrahydro-5-Pyrimidinecarboxylate:

A mixture of 3.02 g (6.33 mmol) 5-methyl 1-(4-nitrophenyl) (6S)-6-(3,4-difluorophenyl)-4-(methoxymethyl)-2-oxo-3,6-dihydro-1,5(2H)-pyrimidinedicarboxylate, 1.50 g (5.80 mmol) of 3-(4-(3-nitrophenyl)-3,6-dihydro-1(2H)-pyridinyl)-1-propanamine, 7.94 g (75.5 mmol) of K₂CO₃ and 1.00 mL of methanol in 200 mL dichloromethane (under argon) was stirred at room temperature for 1 hour. The reaction mixture was filtered and concentrated in vacuo. The residue was dissolved in 100 mL of ethyl acetate and washed 3×50 mL of 5% aqueous NaOH solution, the organic layer was dried (MgSO₄) and concentrated in vacuo. The residue was dissolved in 100 mL of anhydrous ethanol containing 0.50 g 10% Pd/C and the reaction mixture was stirred under a hydrogen balloon for 24 hours. The reaction mixture was passed through a column of Celite 545 filtering agent, washed with ethanol, the filtrate was dried (MgSO₄) and concentrated in vacuo. The residue was purified by column chromatography (silica, 9.5:0.5 ,dichloromethane:methanol+1% isopropyl amine) to afford 1.65 g (52.0% yield) of the desired product.

Tert-Butyl 4-[3-(Isobutyrylamino)Phenyl]-3,6-Dihydro-1(2H)-Pyridinecarboxylate:

Into a solution of 4.00 g (16.0 mmol) of tert-butyl 4-(3-aminophenyl)-3,6-dihydro-1(2H)-pyridinecarboxylate and 5.60 mL (32.0 mmol) of diisopropylethylamine in 100 mL dichloromethane was slowly added 1.90 mL (19.0 mmol) of isobutyryl chloride. The reaction mixture was stirred at room temperature for 2 hours, washed with water, dried (MgSO₄), and concentrated in vacuo. The residue was purified by column chromatography (silica, 50:46:3:1, hexanes:dichloromethane:methanol:isopropyl amine) to afford 2.90 g (52.0% yield) of the desired product: ¹H NMR (400 MHz, CDCl₃) δ 7.69 (s, 1H), 7.34 (d, 1H, J=7.8 Hz), 7.27 (t, 1H, J=7.8 Hz), 7.11 (d, 1H, J=7.8 Hz), 6.04 (s, 1H), 4.05 (s, 2H), 3.62 (apparent t, 2H, J=4.9 Hz), 2.51 (m, 3H), 1.49 (s, 9H), 1.25 (d, 6H, J=7.4 Hz); ESMS m/e: 345.5 (M+H)⁺. Anal. Calc. for C₂₀H₂₈N₂O₃+0.175 CHCl₃: C, 66.33; H, 7.77; N, 7.67. Found: C, 66.20; H, 7.41; N, 7.88

Tert-Butyl 4-[3-(Isobutyrylamino)Phenyl]-1-Piperidinecarboxylate:

A mixture of 2.90 g (8.40 mmol) of tert-butyl 4-[3-(isobutyrylamino)phenyl]-3,6-dihydro-1(2H)-pyridinecarboxylate and 0.80 g of 10% yield Pd/C in 100 mL of ethanol was stirred under a hydrogen balloon for 24 hours. The reaction mixture was passed through a column of Celite 545 filtering agent, the filtrate was dried (MgSO4) and concentrated in vacuo. The residue was purified by column chromatography (silica, 9.5:0.5, dichloromethane:methanol+1% isopropyl amine) to afford 2.40 g (84.0% yield) of the desired product: ¹H NMR (400 MHz, CDCl₃) δ 7.49-7.44 (m, 2H), 7.24 (t, 1H, J=7.6 Hz), 6.93 (d, 1H, J=7.6 Hz), 4.20-4.10 (m, 2H), 2.86-2.45 (m, 4H), 1.86-1.75 (m, 4H), 1.48 (s, 9H), 1.24 (d, 6H, J=6.8 Hz); ESMS m/e: 345.2 (M+H)⁺; Anal. Calc. for C₂₀H₃₀N₂O_{3+0.3}H₂O: C, 68.27; H, 8.77; N, 7.96. Found: C, 68.25; H, 8.54; N, 7.84.

2-Methyl-N-[3-(4-Piperidinyl)Phenyl]Propanamide:

Into a stirred solution of 2.20 (6.50 mmol) of tert-butyl 4-[3-(isobutyrylamino)phenyl]-1-piperidinecarboxylate in 100 ml of 1,4-dioxane at 0° C. was bubbled HCl gas for 10 minutes. The reaction mixture was allowed to warm to room temperature and the bubbling of the HCl gas was continued for 1 hour. The solvent was removed in vacuo, the residue was dissolved in 50 mL of water and was neutralized by the addition of KOH pellets. The aqueous solution was extracted with 3×80 mL of dichloromethane, the combined organic extracts were dried (MgSO₄), filtered and concentrated in vacuo. The residue was purified by column chromatography (silica, 9:1, dichloromethane:methanol+1% isopropyl amine) to afford 0.700 g (46.0% yield) of the desired product: ¹H NMR (400 MHz, CDCl₃) δ 7.47 (s, 1H), 7.40 (d, 1H, J=7.8 Hz), 7.24 (t, 1H, J=7.8 Hz), 7.00 (d, 1H, J=7.8 Hz), 3.23-3.14 (m, 5H), 2.82-2.57 (m, 4H), 1.20 (d, 6H, J=6.8 Hz); ESMS m/e: 247.2 (M+H)⁺;

The hydrochloride salt was used for the combustion analysis: Anal. Calc. for C₁₅H₂₂N₂O+HCl+0.15 CHCl₃: C, 60.51; H, 7.76; N, 9.32. Found: C, 60.57; H, 7.83; N, 8.88.

3-(4-Piperidinyl)Aniline:

¹H NMR (400 MHz, CDCl₃) δ 7.01 (t, 1H, J=7.6 Hz), 6.62-6.54 (m, 3H), 3.16 (br d, 2H, J=10.3 Hz), 2.75 (dt, 2H, J=2.7, 12.3 Hz), 2.56 (tt, 1H, J=3.6, 12.3 Hz), 1.81 (br d, 2H, J=12.3 Hz), 1.65 (dq, 2H, J=4.0, 12.3 Hz); ESMS m/e: 177.2 (M+H)⁺.

Tert-Butyl 4-(4-Nitrophenyl)-3,6-Dihydro-1(2H)-Pyridinecarboxylate:

To a 25-mL RB flask, equipped with a condensor, was added tert-butyl 4-{[(trifluoromethyl)sulfonyl]oxy}-3,6-dihydro-1(2H)-pyridinecarboxylate (1.0 g), 4-nitrophenylboronic acid (0.71 g), sodium carbonate (0.430 mL of 2M solution), lithium chloride (0.382 g), tetrakis(triphenylphosphine)-palladium (0) (0.173 g) and ethylene glycol dimethyl ether (10 mL). The reaction mixture was flushed with Argon three times, then the reaction mixture was heated to 100° C. for 3 hrs. After cooling to room temperature, the reaction mixture was diluted with methylene chloride (30 mL) and water (30 mL) and the organic layer was separated. The aqueous layer was extracted with methylene chloride (3×20 mL) and the combined organic extracts were washed with sat NH₄Cl (20 mL) and brine (20 mL), dried over MgSO₄ and concentrated under reduced pressure. The residue was purified by chromatography (6:1=hexane:ethyl acetate with 1% NH₃) to afford the product (0.55 g, 59.9%) as a yellow oil. The compound is not stable at room temperature and should be used as prompt as practical: ¹H NMR (400 MHz, CDCl₃) δ 8.20 (d, 2H, J=8.6 Hz), 7.51 (d, 2H, J=8.6 Hz), 6.24 (m, 1H), 4.13 (m, 2H), 3.67 (apparent t, 2H, J=5.5 Hz), 2.55 (m, 2H), 1.49 (s, 9H).

4-(4-Nitrophenyl)-1,2,3,6-Tetrahydropyridine:

4-(4-Nitrophenyl)-1,2,3,6-tetrahydropyridine was prepared by a similar procedure to that used for the preparation of 2-methyl-N-[3-(4-piperidinyl)phenyl]propanamide using HCl gas and tert-Butyl 4-(4-Nitrophenyl)-3,6-dihydro-1(2H)-pyridinecarboxylate (130 mg) in dioxane (5.0 mL) at room temperature. The reaction mixture was concentrated in vacuo to give the crude product (69.8 mg) that used in the next reaction without further purification.

Dihydropyrimidine Intermediates

3-(3,4,5-Trifluorobenzylidene)-2,4-Pentanedione:

A stirring mixture of 3,4,5-trifluorobenzaldehyde (4.20 g, 26.2 mmol), 2,4-pentanedione (2.62 g, 26.2 mmol), piperidine (0.430 g, 5.00 mmol) in benzene (150 mL) was heated at reflux temperature in a Dean-Stark apparatus for 8 h. The benzene was evaporated and the yellow oily residue was used in the next step without further purification.

1-[2-Methoxy-4-Methyl-6-(3,4,5-Trifluorophenyl)-1,6-Dihydro-5-Pyrimidinyl]Ethanone:

A mixture 3-(3,4,5-trifluorobenzylidene)-2,4-pentanedione (26.2 mmol), O-methylisourea hydrogen sulfate (3.22 g, 39.3 mmol), and NaHCO₃ (6.6 g, 78.6 mmol) in EtOH (400 mL) was stirred and heated at 95-100° C. for 6 h. The mixture was filtered and the solid filter cake was washed with ethanol (100 mL). The solvent was evaporated from the combined filtrates and the crude product was purified by flash column chromatography (EtOAc/hexane, 1/9 to 1/4), to afford the desired product as an oil (2.80 g, 36%).

4-Nitrophenyl 5-Acetyl-2-Methoxy-4-Methyl-6-(3,4,5-Trifluorophenyl)-1(6H)-Pyrimidinecarboxylate:

4-Nitrophenyl chloroformate (1.89 g, 9.38 mmol) was added to a solution of 1-[2-methoxy-4-methyl-6-(3,4,5-trifluorophenyl)-1,6-dihydro-5-pyrimidinyl]ethanone (2.80 g, 9.38 mmol) and pyridine (10 mL) in CH₂Cl₂ (200 mL) at 0-5° C., and the resulting mixture was allowed to warm to room temperature. After 12 h, the solvent was evaporated and the residue was purified by flash chromatography (dichloromethane/EtOAc, 1/9 to 3/20), to give the desired product as a white powder (4.00 g, 92%).

4-Nitrophenyl 5-Acetyl-4-Methyl-2-oxo-6-(3,4,5-Trifluorophenyl)-3,6-Dihydro-1(2H)-Pyrimidinecarboxylate:

A solution of 6 N aqueous HCl (4 mL) was added to a well-stirred solution of 4-nitrophenyl 5-acetyl-2-methoxy-4-methyl-6-(3,4,5-trifluorophenyl)-1(6H)-pyrimidinecarboxylate (4.00 g, 8.63 mmol) in THF (100 mL) at 0-5° C., and the mixture was allowed to warm to room temperature. After 2 h, solvent was evaporated and the product dried under vacuum. The product was obtained as a pure single component and used in the next step without further purification (3.88 g, 100%).

:¹H NMR (DMSO) δ 10.29 (s, 1H), 8.23 (d, 2H, J=9.1 Hz), 7.51 (d, 2H, J=9.1 Hz), 7.15-7.07 (m, 2H), 6.18 (s, 1H), 2.30 (s, 3H), 2.28 (s, 3H); ESMS m/e: 450.2 (M+H)⁺; Anal. Calc. for C₂₀H₁₄F₃N₃O₆: C, 53.46; H, 3.14; N, 9.35. Found: C, 53.26; H, 3.21; N, 9.35.

Benzyl 2-Propionyl-3-(3,4,5-Trifluorophenyl)-2-Propenoate.

A solution of benzyl propionylacetate (36.3 g, 176 mmol), 3,4-difluorobenzaldehyde (25.0 g, 176 mmol), piperidine (0.86 mL, 9.0 mmol) and acetic acid (0.49 mL, 9.0 mmol) were heated at reflux temperature with removal of water using a Dean-Stark apparatus for 5h. The solvent was removed in vacuo and the residue was dissolved in EtOAc. The organic layer was washed with water (100 mL) followed by brine (100 mL) and dried over anhydrous Na₂SO₄. The solvent was evaporated to afford a pale yellow syrup (60.2 g), which was used in the next step without further purification.

Benzyl 6-(3,4-Difluorophenyl)-4-Ethyl-2-Methoxy-1,6-Dihydro-5-Pyrimidinecarboxylate.

A suspension of benzyl 2-propionyl-3-(3,4,5-trifluorophenyl)-2-propenoate (16.0 g, 48.0 mmol), O-methylisourea hydrogen sulfate (16.65 g, 97.02 mmol), NaHCO₃ (16.3 g, 130.2 mmol) in DMF (190 mL) was stirred at 70° C. for 20h. After cooling to room temperature, the reaction mixture was filtered and the filtrate was diluted with EtOAc (300 mL) and then washed with water (4×100 mL), brine (200 mL) and dried over Na₂SO₄. After removal of solvent, the residue was purified by column chromatography (SiO₂, EtOAc/Hexane, 10%-30%) to afford benzyl 6-(3,4-difluorophenyl)-4-ethyl-2-methoxy-1,6-dihydro-5-pyrimidinecarboxylate as a colorless oil (10.6 g, 58% yield). The product was directly used in the next step after ¹H NMR spectroscopy which showed it to be a mixture of amine/imine tautomers.

5-Benzyl 1-(4-nitrophenyl) 6-(3,4-Difluorophenyl)-4-Ethyl-2-Methoxy-1,5(6H)-Pyrimidinedicarboxylate.

Into a well-stirred solution of benzyl 6-(3,4-difluorophenyl)-4-ethyl-2-methoxy-1,6-dihydro-5-pyrimidinecarboxylate (27.5 g, 68.75 mmol) and pyridine (9.2 mL) in CH₂Cl₂ (300 mL) was added 4-nitrophenyl chloroformate (14.49 g, 82.5 mmol) at room temperature. The reaction mixture was stirred for 4 h and then washed with 10% aqueous KOH solution (2×150 mL). The organic layer was separated and dried over Na₂SO₄. The solvent was removed in vacuo and the residue was used in the next step without further purification: ¹H NMR (CDCl₃) δ 1.24 (t, J=7.2 Hz, 3H), 2.81-2.98 (m, 3H), 3.97 (s, 3H), 5.14 (AB_{q}, 2H), 6.28 (s, 3H), 7.03-7.29 (m, 8H), 7.35 (d, J=9.2 Hz, 2H), 8.26 (d, J=9.2 Hz, 2H).

Benzyl 6-(3,4-Difluorophenyl)-4-Ethyl-2-Methoxy-1-({[(1R)-1-Phenylethyl]Amino}Carbonyl)-1,6-Dihydro-5-Pyrimidinecarboxylate.

Into a stirred mixture of 5-benzyl 1-(4-nitrophenyl) 6-(3,4-difluorophenyl)-4-ethyl-2-methoxy-1,5(6H)-pyrimidinedicarboxylate (12.6 g, 22.86 mmol) in THF (150 mL) was added a solution of R-(+)-α-methyl benzylamine (3.53 mL, 27.44 mmol) at room temperature. The stirring was continued for 12 h and the solvent was removed in vacuo. The yellow residue was dissolved in chloroform (200 mL) and was washed with 10% K₂CO₃ solution (2×30 mL). The organic layer was dried over Na₂SO₄, filtered and the solvent was removed in vacuo. The resulting mixture of diastereomers was separated by column chromatography over silica gel with 9:1 pet. ether:ether to 4:1 pet. ether:ether. First major product to elute was (+)-benzyl 6-(3,4-difluorophenyl)-4-ethyl-2-methoxy-1-({[(1R)-1-phenylethyl]amino}carbonyl)-1,6-dihydro-5-pyrimidinecarboxylate: Colorless oil, Rf=0.31(4:1 pet ether:ether); wt.=3.8 g (60% yield); [α]_{D}=+267.05 (c=0.76, CHCl₃); ¹H NMR (CDCl₃) δ 1.22 (t, J=7.5 Hz, 3H), 1.52 (d, J=6.9 Hz, 3H), 2.88 (q, J=6.0 Hz, 2H), 3.99 (s, 3H), 4.99 (m, 1H), 5.09 (AB_{q}, 2H), 6.66 (s, 1H), 6.99-7.36 (m, 13H); The second major product to elute was (−)-benzyl 6-(3,4-difluorophenyl)-4-ethyl-2-methoxy-1-({[(1R)-1-phenylethyl]amino}carbonyl)-1,6-dihydro-5-pyrimidinecarboxylate: Colorless oil; R_{f}=0.22 (4:1 pet ether:ether); wt.=3.2 g (51.2% yield); [α]_{D}=−146.89 (c=0.38, CHCl₃); ¹H NMR (CDCl₃) δ 1.22 (t, J=7.2 Hz, 3H), 1.49 (d, J=6.6 Hz, 3H), 2.88 (q, J=6.0 Hz, 2H), 3.94 (s, 3H), 5.03 (m, 1H), 5.11 (AB_{q}, 2H), 6.68 (s, 1H), 6.91-7.34 (m, 13H).

(+)-Benzyl 6-(3,4-Difluorophenyl)-4-ethyl - 94 -2-Methoxy-1,6-Dihydro-5-Pyrimidinecarboxylate.

Into a stirred solution of (+)-benzyl 6-(3,4-difluorophenyl)-4-ethyl-2-methoxy-1-({[(1R)-1-phenylethyl]amino}carbonyl)-1,6-dihydro-5-pyrimidinecarboxylate (17.1 mmol, 9.35 g) in CH₂Cl₂ was added 1,8-diazabicyclo[5,4,0]-undec-7-ene (17.1 mmol, 2.56 mL) and stirring was continued for 16 h at room temperature. The solvent was evaporated and the residue was purified by flash column chromatography on silica gel with 3:1 EtOAc/Hexanes as the eluting system. 5.27 g of the (+)-benzyl 6-(3,4-difluorophenyl)-4-ethyl-2-methoxy-1,6-dihydro-5-pyrimidinecarboxylate was obtained (77% yield).

(+)-5-Benzyl 1-(4-Nitrophenyl) 6-(3,4-Difluorophenyl)-4-Ethyl-2-Methoxy-1,5(6H)-Pyrimidinedicarboxylate.

Into a well-stirred solution of (+)-benzyl 6-(3,4-difluorophenyl)-4-ethyl-2-methoxy-1,6-dihydro-5-pyrimidinecarboxylate (6.4 g, 16.0 mmol) and pyridine (1.5 mL) in CH₂Cl₂ (150 mL) was added 4-nitrophenyl chloroformate (3.41 g, 19.2 mmol) at room temperature. The reaction mixture was stirred for 4 h and then it was washed with 10% aqueous KOH solution (2×100 mL). The organic layer was separated and dried over Na₂SO₄. The solvent was removed in vacuo. The residue of (+)-5-benzyl 1-(4-nitrophenyl) 6-(3,4-difluorophenyl)-4-ethyl-2-methoxy-1,5(6H)-pyrimidinedicarboxylate was used in the next step without further purification.

a. 2-(4-Methoxybenzyl)-2-Thiopseudourea Hydrochloride.

Into a well-stirred suspension of thiourea (7.6 g, 0.1 mol) in THF (50 mL) at 0° C., 4-methoxybenzyl chloride (16 g, 0.1 mol) was added in 10 min and the reaction mixture was allowed to warm to room temperature. After 2 hours the reaction mixture was heated to 65° C. and kept at that temperature for 5 hours. The reaction mixture was cooled to room temperature and diluted with diethyl ether (200 mL). The white precipitate that formed was filtered and dried (22.5 g, 96% yield); m. p. 161-163° C.

b. Methyl 2-{(4-Nitrophenyl)Methylene)-3-Oxobutyrate.

A mixture of 4-nitrobenzaldehyde (15.1 g, 0.1 mol), methyl acetoacetate (12.773 g, 0.11 mol), piperidine (0.41 g, 4.80 mmol), and acetic acid (0.288 g, 4.8 mmol) in 2-propanol (400 mL) was stirred at room temperature for 48 hours. The resulting white solid, methyl 2-{(4-nitrophenyl)methylene}-3-oxobutyrate was filtered, washed with 2-propanol (2×50 mL) and dried (21.8 g, 93% yield).

c. 1,6-Dihydro-5-Methoxycarbonyl-2-[{(4-Methoxyphenyl)Methy l}Thio]-4-Methyl-6-(4-Nitrophenyl)Pyrimidine.

A mixture of methyl 2-{(4-nitrophenyl)methylene}-3-oxobutyrate (8.96 g, 0.04 mol), 2-(4-methoxybenzyl)-2-thiopseudourea hydrochloride (9.28 g, 0.04 mol), and NaOAc (3.28 g, 0.04 mol) in DMF (100 mL) was stirred and heated at 70-75° C. for 4.5 hours. The reaction mixture was cooled to room temperature, poured into ice-water (300 mL) and extracted with EtOAc (2×400 mL). The combined EtOAc extracts were washed with 10% NaHCO₃ solution (2×60 mL), brine (100 mL), and then dried (MgSO₄). The solvent was evaporated and the crude product was purified by flash column chromatography on silica gel using 10% through 30% EtOAc in hexane as the gradient eluent. The desired product was obtained as an oil, which on trituration with EtOAc/hexane became a yellow solid (11.4 g, 66.7% yield) which was shown by ¹H NMR to be a mixture of tautomers: m.p. 138-139° C.; 1H NMR (CDCl₃) δ 2.15 (s, 3H), 3.62 (s, 3H), 3.72 (s, 3H), 4.05 and 5.78 (s and d, J=3 Hz, 1H), 4.08, 4.20 (AB q, J=12.5 Hz, 2H), 4.21 and 6.40 (s and d, J=3 Hz, 1H), 6.66 (2 d, J=8.5 Hz, 2H), 7.08 (2 d, J=8.5 Hz, 2H), 7.37 (2 d, J=8.8 Hz, 2H), 8.7 (2 d, J=8.8 Hz, 2H); Anal. Calcd. for C₂₁H₂₁N₃O₅S: C, 59.00; H, 4.95; N, 9.83. Found: C, 59.02; H, 4.93; N, 9.77.

d. 1,6-Dihydro-5-Methoxycarbonyl-2-[{(4-Methoxyphenyl) Methyl}Thio]-4-Methyl-6-(4-Nitrophenyl)-1-[(4-Nitropheny loxy)Carbonyl]Pyrimidine.

Into a well-stirred mixture of 1,6-dihydro-5-methoxy carbonyl-2-[{(4-methoxyphenyl)methyl}thio]-4-methyl-6-(4 -nitrophenyl)pyrimidine (4.50 g, 10.5 mmol), NaHCO₃ (3.69 g, 0.044 mol), CH₂Cl₂ (200 mL), and water (50 mL) at 0-5° C., 4-nitrophenyl chloroformate (2.40 g, 12.0 mmol) was added over a 5 min period and the reaction mixture was allowed to warm to room temperature. After 10 hours, the TLC analysis of the reaction mixture showed the presence of a small amount of starting pyrimidine, therefore, more 4-nitrophenyl chloroformate (0.65 g, 0.0032 mol) was added and the stirring was continued for an additional 4 hours. The two layers were separated, the CH₂Cl₂ layer was washed with saturated aqueous NaHCO₃ solution (3×50 mL), dried (MgSO₄), and the solvent evaporated. The residue was recrystallized from CH₂Cl₂ and hexane to give the product as white crystals (5.50 g, 88.4% yield): m.p. 156-157° C.; ¹H-NMR (CDCl₃) δ 2.53 (s, 3H), 3.70 (s, 3H), 3.81 (s, 3H), 4.06, 4.36 (ABq, J=13.5 Hz, 2H), 6.30 (s, 1H), 6.78 (d, J=8.6 Hz, 2H), 7.17 (d, J=8.6 Hz, 2H), 7.20 (d, J=8.8 Hz, 2H), 7.32 (d, J=8.8 Hz, 2H), 7.97 (d, J=8.8 Hz, 2H), 8.25 (d, J=8.8 Hz, 2H); Anal. Calcd. for C₂₈H₂₄N₄O₉S: C, 56.75; H, 4.08; N, 9.45. Found: C, 56.49; H, 4.28; N, 9.25.

a. 6-(Benzofurazan-5-yl)-1,6-Dihydro-2-oxo-5-Methoxycarbonyl-4-Bromomethyl-1-[(4-Nitrophenyl-oxy)Carbonyl]Pyrimidine.

Into a well-stirred solution of 6-(benzofurazan-5-yl)-1,6-dihydro-2-methoxy-5-methoxycarbonyl-4-methyl-1-[(4-nitrophenyl-oxy)carbonyl]pyrimidine (0.310 mmol, 0.140 g) in 1.5 mL of chloroform was added a solution of bromine (0.310 mmol, 0.020 mL) in 1.5 mL of chloroform at 0° C. and the solution was allowed to attain room temperature over 1.5 h. The solvent was removed in vacuo and the residue was again dissolved in CHCl₃ (10 mL) and washed with brine. The organic layer was separated, dried over Na₂SO₄, filtered and the solvent was removed in vacuo to obtain 0.15 g (88% yield) of 6-(benzofurazan-5-yl)-1,6-dihydro-2-oxo-5-methoxycarbonyl-4-bromomethyl-1-[(4-nitrophenyl-oxy)carbonyl]pyrimidine as a yellow foam. The crude product was used in the next step without purification. ¹H NMR (CDCl₃) δ 3.79 (s, 3H), 4.72 (ABq, 2H), 6.47 (s, 1H), 7.37 (d, J=9.1 Hz, 2H), 7.51 (d, J=7.8 Hz, 1H), 7.80 (s, 1H), 7.92 (d, J=9.1 Hz, 1H), 8.30 (d, J=9.1 Hz, 2H)

c. 4-Nitrophenyl 4-(2,1,3-Benzoxadiazol-5-yl)-2,5-Dioxo-1,2,5,7-Tetrahydrofuro[3,4-D]Pyrimidine-3(4H)-Carboxylate.

6-(3,4-Benzofurazan-5-yl)-1,6-dihydro-2-oxo-5-methoxy-carbonyl-4-bromomethyl-1-[(4-nitrophenyloxy)carbonyl]pyrimidine (0.27 mmol, 0.15 g) was heated in oil bath for 3 h (bath temperature 130° C. The brownish-yellow residue thus obtained was washed with CHCl₃ and 4-nitrophenyl 4-(2,1,3-benzoxadiazol-5-yl)-2,5-dioxo-1,2,5,7-tetrahydrofuro[3,4-d]pyrimidine-3(4H)-carboxylate was obtained as an off-white solid which was used in the next step without further purification (crude wt. 0.11 g, 93% yield): H NMR (DMSO-d₆) δ 8.38-7.56 (m, 7H), 6.33 (s, 1H), 5.02 (s, 2H) Anal. Calc. for C₁₉H₁₁N₅O₈+2.3H₂O: C, 47.85; H, 3.28; N, 14.63. Found: C, 47.73; H, 2.51; N, 14.77.

5-Methyl 1-(4-Nitrophenyl) 4-(Bromomethyl)-6-(3,4-Difluorophenyl)-2-oxo-3,6-Dihydro-1,5(2H)-Pyrimidinedicarboxylate:

Into a well-stirred solution of 6-(3,4-Difluorophenyl)-1,6-dihydro-2-methoxy-5-methoxycarbonyl-4-methyl-1-[(4-nitrophenyloxy)carbonyl]pyrimidine (1.5 mmol, 0.66 g) in 5 mL of chloroform was added a solution of bromine (1.5 mmol, 0.09 mL) in 3 mL of chloroform at 0° C. and the solution was allowed to attain room temperature over 1.5 h. The solvent was removed in vacuo and the residue was again dissolved in CHCl₃ (20 mL) and washed with brine. The organic layer was separated, dried over Na₂SO₄, filtered and the solvent was removed in vacuo to afford the desired product as a yellow foam, which was used in the next step without purification. ¹H NMR δ 3.75 (s, 3H), 4.67 (ABq, 2H), 6.35 (s, 1H), 7.09-7.19 (m, 4H), 7.37 (d, J=9.0 Hz, 2H), 8.27 (d, J=9.0 Hz, 2H).

4-Nitrophenyl 4-(3,4-Difluorophenyl)-2,5-Dioxo-1,2,5,7-Tetrahydrofuro[3,4-D]Pyrimidine-3(4H)-Carboxylate.

5-methyl 1-(4-nitrophenyl) 4-(bromomethyl)-6-(3,4-difluorophenyl)-2-oxo-3,6-dihydro-1,5(2H)-pyrimidinedicarboxylate (1.5 mmol, 0.81 g) was heated in an oil bath for 3 h (bath temperature 130° C.). The brown residue thus obtained was washed with CHCl₃ and the desired product was obtained as a pale brown solid which was used in the next step without further purification (crude wt. 0.51 g): ¹H NMR (DMSO-d₆) δ 4.94 (br s, 2H), 6.08 (s, 1H), 7.20-7.43 (m, 4H), 8.35 (d, J=10.2 Hz, 2H).

4-Nitrophenyl 4-(1,3-Benzodioxol-5-yl)-2,5-Dioxohexahydrofuro[3,4-D]Pyrimidine-3(4H)-Carboxylate:

¹H NMR (DMSO) δ 11.35 (s, 1H), 8.16 (d, 2H, J=9.5 Hz), 7.32 (d, 2H, J=8.9 Hz), 6.81-6.65 (m, 3H), 5.88 (s, 1H), 4.85 (ABq, 2H); ESMS m/e: 440.1 (M+H)⁺; Anal. Calc. for C₂₀H₁₅N₃O₉+1.5H₂O: C, 51.29; H, 3.87; N, 8.97. Found: C, 51.38; H, 2.85; N, 8.73.

5-Methyl 1-(4-Nitrophenyl) (6S)-6-(3,4-Difluorophenyl)-4-Methyl-2-oxo-3,6-DIHYDRO-1,5(2H)-Pyrimidinedicarboxylate:

¹H NMR (400 MHz, CDCl₃) δ 8.29 (d, 2H, J=9.1 Hz), 7.36 (d, 2H, J=8.9 Hz), 7.25-7.11 (m, 3H), 6.37 (s, 1H), 3.75 (s, 3H), 2.46 (s, 3H); ESMS m/e: 448.1 (M+H)⁺; Anal. Calc. for C₂₀H₁₅F₂N₃O₇: C, 53.70; H, 3.38; N, 9.39. Found: C, 53.35; H, 3.36; N, 9.27.

General Procedure for the Reaction of Pyrimidine-3-carboxylic Acid-4-nitrophenyl Esters with Amines:

A solution of substituted pyrimidine-3-carboxylic acid-4-nitrophenyl ester ((0.29 mmol) and a substituted 4-phenyl-1-(3-propylaminopiperidine (0.30 mmol) in 10 mL of anhydrous THF was stirred overnight at room temperature. The solvent was removed in vacuo and the residue was purified by column chromatography.

Tert-Butyl 4-([(Trifluoromethyl)Sulfonyl]OXY}-1,2,3,6-Tetra-Hydro-1Pyridinecarboxylate: n-Butyllithium (17.6 mL, 44.2 mmol, 2.5 M in hexanes) was added to a solution of diisopropyl amine (96.2 mL, 44.2 mmol) in 40 mL of dry THF at 0° C. and stirred for 20 minutes. The reaction mixture was cooled to −78° C. and tert-butyl 4-oxo-1-piperidinecarboxylate (40.0 mmol) in THF (40 mL) was added dropwise to the reaction mixture and stirred for 30 minutes. Tf₂NPh (15.0 g, 42.0 mmol) in THF (40 mL) was added dropwise to the reaction mixture and the mixture was stirred at 0° C. overnight. The reaction mixture was concentrated in vacuo, re-dissolved in hexanes/EtOAc (9/1), passed through a plug of alumina and washed with hexanes/EtOAc (9/1). The combined extracts were concentrated to yield 16.5 g of the desired product that was contaminated with a small amount of Tf₂ Nph. ¹H NMR δ 5.77 (s, 1H), 4.05 (dm, 2H, J=3.0 Hz), 3.63 (t, 2H, J=5.7 Hz), 2.45 (m, 2H), 1.47 (s, 9H).

Tert-Butyl 4-[3-(Acetylamino)Phenyl]-1,2,3,6-Tetrahydro-1-Pyridinecarboxylate:

A mixture of saturated of aqueous Na₂CO 3 solution (25 mL), tert-butyl 4-{[(trifluoromethyl)sulfonyl]oxy}-1,2,3,6-tetrahydro-1-pyridine-carboxylate (20 mmol), 3-acet-amidophenylboronic acid (30 mmol) and tetrakis-triphenylphosphine palladium (0) (1.15 g) and dimethoxyethane (40 mL) was heated at reflux temperature overnight. The organic layer of the cooled reaction mixture was separated and the aqueous layer was washed with ethyl acetate (3×). The combined organic extracts were dried and concentrated in vacuo. The crude product was chromatograghed, giving the desired product ¹H NMR δ 8.11 (br s, 1H), 7.57 (br s, 1H), 7.41 (brδ, 1H, J=7.8 Hz), 7.25 (apparent t, 1 H, J=7.8 Hz), 7.08 (br d, 1H, J=7.8 Hz), 5.99 (b s, 1H), 4.03 (br m, 2H, J=2.7 Hz), 3.59 (t, 2H, J=5.7 Hz), 2.46 (m, 2H,), 2.16 (s, 3H), 1.49 (s, 9H).

N1-[3-(1,2,3,6-Tetrahydro-4-Pyridinyl)Phenyl]Acetamide:

A solution of 4 M HCl in dioxane (10 mL) was added to tert-butyl 4-[3-(acetylamino)phenyl]-1,2,3,6-tetrahydro-1-pyridinecarboxyl-ate (8.25 mmol) in dichloromethane (30 mL). The reaction mixture was stirred at room temperature overnight, concentrated in vacuo, giving the desired product as the hydrochloride salt (2.1 g). ¹H NMR δ 7.41-7.00 (m, 4H), 6.10 (br, 1H), 3.55 (m, 2H), 3.16 (t, 2H, J=5.7 Hz), 2.44 (m, 2H), 2.19 (s, 3H).

Tert-Butyl N-(3-Bromopropyl)Carbamate:

Prepared from 3-bromopropylamine hydrobromide and BOC₂O in the presence of base in dichloromethane: ¹H NMR δ 5.07 (br, 1H), 3.31 (t, 2H, J=6.6 Hz), 3.12 (apparent br q, 2H, J=6.0 Hz), 1.92 (p, 2H, J=6.6 Hz), 1.30 (s, 9H).

Reaction of N1-[3-(1,2,3,6-Tetrahydro-4-Pyridinyl)Phenyl]Acetamide with Tert-Butyl N-(3-Bromopropyl)Carbamate Tert-Butyl N-(3-{4-[3-(Acetylamino)Phenyl]-1,2,3,6-Tetrahydro-1-Pyridinyl}Propyl)Carbamate:

A solution of N1-[3-(1,2,3,6-tetrahydro-4-pyridinyl)phenyl]acetamide hydrochloride (8.24 mmol), tert-butyl N-(3-bromopropyl)carbamate and potassium carbonate (33 mmol) in dry dioxane (30 mL) was heated at reflux temperature overnight. The solids were removed by filtration, the solution was concentrated in vacuo and the product was chromatographed, giving the desired product (110 mg). ¹H NMR δ 7.65 (s, 1H), 6.98 (s, 1H), 7.45 (d, 1H, J=7.8 Hz), 7.16 (apparent t, 1H, J=7.8 Hz), 7.10 (d, 1H, J=7.8 Hz), 6.02 (s, 1H), 5.23 (b, 1H), 3.40 (b, 2H), 3.30-1.80 (m, 10H), 2.18 (s, 3H), 1.45 (s, 9H)

Deprotection of BOC:

N1-{3-[1-(3-Aminopropyl)-1,2,3,6-Tetrahydro-4-Pyridinyl]Phenyl}Acetamide:

A 1:1 solution of TFA:CH₂Cl₂ (5 mL) was added to tert-butyl N-(3-{4-[3-(acetylamino) phenyl]-1,2,3,6-tetrahydro-1-pyridinyl}propel)carbamate in dichloromethane (5 mL). The resulting solution was stirred at room temperature for 1-3 days, saturated NaHCO3 was added until pH>6, the organic layer was separated, and dried in vacuo, giving the desired product (45 mg): ¹H NMR δ 7.68 (br, 1H), 7.35 (dm, 1H, J=7.8 Hz), 7.25 (apparent t, 1H, J=7.8 Hz), 7.15 (dm, 1H, J=7.8 Hz), 6.12 (m, 1H), 3.22 (m, 2H), 3.03 (t, 2H, J=7.3 Hz), 2.78 (t, 2H, J=5.5 Hz), 2.70-2.50 (m, 4H), 2.10 (s, 3H), 1.87 (p, 2H, J=7.3 Hz).

Tert-Butyl 4-[3-(Acetylamino)Phenyl]-1-Piperidinecarboxylate:

A mixture tert-butyl 4-[3-(acetylamino)phenyl]-1,2,3,6-tetra-hydro-1-pyridinecarboxylate (710 mg) and 5% Pd/C (100 mg) in EtOH (10 mL) was hydrogenated (balloon technique) at room temperature overnight. The reaction mixture was passed through a pad of Celite 545 and the pad of Celite was washed with ethanol. The combined ethanol extracts were concentrated and chromatograghed, giving the desired product (660 mg). ¹H NMR δ 7.80 (s, 1H), 7.41-7.20 (m, 3H), 6.94 (d, 1H, J=7.5 Hz), 4.21 (m, 2H), 2.75 (m, 2H), 2.62 (m, 1H), 2.16 (s, 3H), 1.78 (m, 2H), 1.56 (m, 2H), 1.48 (s, 9H)

N1-[3-(4-Piperidyl)Phenyl]Acetamide:

A solution of HCl in dioxane (4N, 5 ML) was added to tert-butyl 4-[3-(acetylamino)-phenyl]-1-piperidinecarboxylate (660 mg) in dry dichloromethane (15 mL). The reaction mixture was stirred at room temperature overnight and concentrated in vacuo, giving the desired product (550 mg): mp 102-104° C.; ¹H NMR δ 2.02 (d, J=13.2 Hz, 2H), 2.11-2.45 (m, 5H), 2.67-2.77 (m, 1H), 3.00-3.10 (m, 2H), 3.51 (d, J=10.5 Hz, 2H), 6.94 (d, J=7.5 Hz, 1H), 7.20-7.46 (m, 3H), 7.60 (s, 1H).

Tert-Butyl N-(3-{4-[3-(Acetylamino)Phenyl]Piperidino}Propyl)-Carbamate:

A solution of N1-[3-(4-piperidyl) phenyl]acetamide (550 mg, 0.210 mmol), tert-butyl N-(3-bromopropyl)-carbamate (550 mg, 0.230 mmol), K₂CO₃ (1.10 g, 0.890 mmol), diisopropylethyl amine (1.50 mL) and a few crystals of KI in dioxane (20 mL) was heated at reflux temperature for 2 days. The precipitated salts were removed by filtration, concentrated in vacuo and the crude product was chromatographed, giving the desired product (340 mg). ¹H NMR δ 8.15 (s, 1H), 7.47-7.44 (m, 2H), 7.22 (t, 1H, J=7.8 Hz), 6.94 (d, 1H, J=7.8 Hz), 5.53 (b, 1H), 3.23 (b, 6H), 2.80-1.60 (m, 9H), 2.20 (s, 3H), 1.45 (s, 9H).

N1-{3-[1-(3-Aminopropyl)-4-Piperidyl]Phenyl}Acetamide:

TFA (1.0 mL) was added to a solution of tert-butyl N -(3-{4-[3-(acetyl-amino)phenyl]piperidino}propyl)carbamate (340 mg) in dry dichloromethane (10 mL) and stirred at room temperature for 5 h. A 10% aqueous solution of KOH was added to the reaction mixture until pH>6 and then the dichloromethane was removed in vacuo. The aqueous layer was frozen and lyophilized, giving a solid which was then extracted with methanol. Removal of methanol gave the desired product (120 mg) as an oil. ¹H NMR δ 8.56-8.46 (s, 1H), 7.43-7.30 (m, 2H), 7.23-7.16 (apparent t, 1H, J=7.5 Hz), 6.95 -6.92 (m, 1H), 3.03-2.99 (m, 2H), 2.77-2.73 (t, 2H, J=6.6 Hz), 2.50-1.60 (m, 10H), 2.13 (s, 3H).

1-Benzyl-4-Hydroxy-4-(4-Fluoro-2-Methylphenyl)Piperidine:

¹H NMR δ 7.40-7.26 (M, 5H), 6.91-6.76 (m, 3H), 3.57 (s, 2H), 2.83-2.72 (m, 2H), 2.61 (s, 3H), 2.58-2.43 (m, 2H), 2.23-2.12 (m, 2H)

1-Benzyl-4-(4-Fluoro-2-Methylphenyl)-1,2,3,6-Tetrahydrop yridine:

¹H NMR δ 7.41-7.26 (m, 5H), 7.05 (dd, 1H, J=6.0, 8.1 Hz), 6.87-6.80 (m, 2H), 5.52-5.50 (m, 2H), 3.65 (s, 2H), 3.13 (q, 2H, J=3.3 Hz), 2.69-2.66 (t, 2H, J=5.1 Hz), 2.35-2.31 (m, 2H), 2.27 (s, 3H)

4-(4-Fluoro-2-Methylphenyl)Piperidine: ¹H NMR δ 7.17 (t, 1H, J=7.2 Hz), 6.83-6.80 (m, 2H), 3.22 (m, 2H), 2.81-2.73 (m, 2H), 2.66 (br s, 1H), 2.33 (s, 3H), 1.80-1.60 (m, 4H).

1-Benzyl-4-(3,4,5-Trifluorophenyl)-1,2,3,6-Tetrahydropyr idine:

¹H NMR δ 7.50-7.20 (m, 7H), 5.67 (m, 1H), 3.69 (s, 2H), 3.19 (apparent q, 2H, J=2.7 Hz), 2.75 (t, 2H, J=5.7 Hz), 2.34 (m, 2H).

4-(3,4,5-Trifluorophenyl)Piperidine:

mp 197-199° C.; 1H NMR δ 2.05 (d, J=13.2 Hz, 2H), ), 2.33 (dd, J=25.5 Hz, J=12.9 Hz, 2H), 3.06-3.23 (m, 3H), 3.73 (d, J=12.0 Hz, 2H), 6.94-7.04 (m, 2H).

4-(3,4,5-Trifluorophenyl)Piperidine:

¹H NMR δ 7.20-6.80 (m, 2H), 3.73 (m, 2H), 3.14 (m, 3H), 2.33 (m, 2H), 2.05 (m, 2H).

Tert-Butyl N-3-[4-(3,4,5-Trifluorophenyl)Piperidino]Propyl-Carbamate:

¹H NMR δ 6.91 (m, 2H), 5.62 (b, 1H), 4.31 (t, 2H, J=5.4 Hz), 3.63 (m, 2H), 3.39 (dt, 2H, J=2.1, 6.0 Hz), 3.40-2.70 (m, 7H), 2.46 (t, 2H, J=6.9 Hz), 2.10-1.60 (m, 4H), 1.45 (s, 9H).

3-[4-(3,4,5-Trifluorophenyl)Piperidino]-1-Propanamine:

¹H NMR δ 6.93 (m, 2H), 4.30 (b, 1H), 3.36 (b, 1H), 3.06 (m, 2H), 2.77 (m, 2H), 2.43 (m, 2H), 2.20-1.40 (m, 9H).

1-Benzyl-4-(5-Fluoro-2-Methoxyphenyl)-4-Piperidinol:

¹H NMR δ 7.40-6.80 (m, 8H), 3.94 and 3.85 (s, 3H), 3.61 and 3.58 (s, 2H), 2.80-1.90 (m, 8H).

1-Benzyl-4-(5-Fluoro-2-Methoxyphenyl)-1,2,3,6-Tetrahydro Pyridine:

¹H NMR δ 7.40-6.70 (m, 8H), 5.84 (m, 1H), 3.77 (s, 3H), 3.64 (s, 2H), 3.17 (m, 2H), 2.68 (t, 2H, J=5.7 Hz), 2.54 (m, 2H).

4-(5-Fluoro-2-Methoxy)Phenyl Piperidine:

mp 254-258° C.; ¹H NMR δ 61.53-1.68 (m, 2H), 1.79 (d, J=11.7 Hz, 2H), 2.12 (dt, J=2.1 Hz, J=11.7 Hz, 1H), 2.77 (dt, J=1.8 Hz, J=12.3 Hz, 1H), 2.90-3.05 (m, 1H), 3.10-3.22 (m, 2H), 3.68 (s, 1H), 3.79 (s, 3H), 6.72-6.93 (m, 3H). Anal. Calcd. For Cl₂H₁₇NOFCl+0.14 CH₂Cl₂: C, 56.60; H, 6.76; N, 5.44. Found: C, 56.60; H, 6.92; N, 5.28.

Tert-Butyl N-3-[4-(5-Fluoro-2-Methoxyphenyl)Piperidino]Propyl-Carbamate:

¹H NMR δ 6.90-6.70 (m, 3H), 5.76 (b, 1H), 3.80 (s, 3H), 3.68 (m, 1H), 3.40-2.90 (m, 4H), 2.45 (t, 2H, J=6.6 Hz), 2.20-1.60 (m, 9H), 1.45 (s, 9H).

3-[4-(5-Fluoro-2-Methoxyphenyl)Piperidino]-1-Propanamine:

¹H NMR δ 7.00-6.80 (m, 3H), 3.80 (s, 3H), 3.05 (d, 2H, J=11.4 Hz), 2.76 (t, 2H, J=6.9 Hz), 2.43 (dd, 2H, J=7.8 Hz), 2.05 (dt, 2H, J=2.4, 11.7 Hz), 1.90-1.20 (m, 10H).

Tert-Butyl 4-(1-Naphthyl)-1,2,3,6-Tetrahydro-1-Pyridinecarboxyl-ate:

¹H NMR δ 8.00-7.80 (m, 2H), 7.76 (d, 1H, J=8.1 Hz), 7.50-7.44 (m, 2H), 7.42 (d, 1H, J=8.1 Hz), 7.27 (d, 1H, J=8.1 Hz), 5.76 (br, 1H), 4.14 (m, 2H), 4 or 3.29 (t, 2H, J=5.7 Hz), 2.52 (br m, 2H), 1.53 (s, 9H).

4-(1-Naphthyl)Piperidine:

HCl salt; mp 330-332° C.; ¹H NMR δ 1.66-1.70 (m, 2H), 2.20-2.26 (m, 2H), 2.30-2.43 (m, 2H), 2.72-2.84 (m, 1H), 3.15-3.26 (m, 2H), 7.42-7.56 (m, 4H), 7.78 (d, J=8.1 Hz, 1H), 7.90 (d, J=8.1 Hz, 1H), 8.04 (d, J=8.1 Hz, 1H). Anal. Calcd. For C₁₅H₁₈NOCl+0.20 CH₂Cl₂: C, 68.96; H, 7.00; N, 5.29. Found: C, 68.64; H, 7.04; N, 5.24.

Tert-Butyl N-3-[4-(1-Naphthyl)Piperidino]Propylcarbamate:

¹H NMR δ 8.09 (d, 1H, J=8.4 Hz), 7.86 (dd, 1H, J=1.8, 7.5 Hz), 7.71 (dd, 1H, J=2.4, 6.9 Hz), 7.60-7.30 (m, 4H), 6.31 (br, 1H), 5.75 (br, 1H), 4.26 (t, 1H, J=5.4 Hz), 3.40-3.00 (m, 6H), 2.54 (t, 2H, J=6.9 Hz), 2.24 (dt, 2H, J=3.0, 11.4 Hz), 2.00-1.60 (m, 6H), 1.45 (s, 9H).

4-(3-Methyl-2-Pyridyl)-4-Piperidinol:

¹H NMR δ 8.21 (dd, 1H, J=1.2, 4.5 Hz), 7.36 (dd, 1H, J=6.6, 7.8 Hz), 7.02 (dd, 1H, J=4.8, 7.5 Hz), 3.07 (dt, 2H, J=2.7, 12.3 Hz), 2.89 (m, 2H), 2.46 (s, 3H), 2.22 (dt, 2H, J=4.8, 12.3 Hz), 1.39 (dm, 2H, J=12.3 Hz).

Tert-Butyl 4-(3-Methyl-2-Pyridyl)-1,2,3,6-Tetrahydro-1-Pyridine-Carboxylate:

¹H NMR δ 8.16 (dd, 1H, J=1.2, 3.3 Hz), 7.51 (dm, 1H, J=7.5 Hz), 7.15 (dd, 1H, J=4.8, 7.5 Hz), 5.73 (br, 1H), 4.01 (m, 2H), 3.59 (t, 2H, J=5.7 Hz), 2.40 (m, 2H), 1.44 (s, 9H)

Tert-Butyl N-3-[4-(3-Methyl-2-Pyridyl)Piperidino]Propylcarbamate: ¹H NMR δ 8.37 (dd, 1H, J=4.2, 4.8 Hz), 7.51 (dd, 1H, J=7.2, 7.5 Hz), 7.20 (dd, 1H, J=4.5, 7.5 Hz), 6.73 (br, 1H), 3.26 (m, 4H), 3.05 (d, 2H, J=12.0 Hz), 2.80-2.40 (m, 4H), 2.61 (s, 3H), 1.82 (p, 2H, J=6.3 Hz), 1.54 (d, 2H, J=12.0 Hz).

Tert-Butyl 4-(3-Methoxyphenyl)-1,2,3,6-Tetrahydro-1-Pyridinecarboxylate:

¹H NMR δ 7.23 (t, 1H, J=8.1 Hz), 6.96 (d, 1H, J=7.5 Hz), 6.89 (d, 1H, J=1.8 Hz), 6.80 (dd, 1H, J=2.4, 8.1 Hz), 6.02 (br, 1H), 4.20-4.00 (m, 3H), 3.80 (s, 3H), 3.62 (t, 2H, J=5.7 Hz), 2.51 (br, 2H), 1.49 (s, 9H).

1-Benzyl-4-Methyl-Piperidin-4-ol:

Methyllithium (1.4 M in Et₂O, 54.0 mL) was added to a solution of 1-benzyl-4-piperidone (5.00 mL, 27.0 mmol) in anhydrous ether at −78° C. under argon. Stirring was continued at −78° C. for 1.5 hours. Ether (200 mL) and water (40 mL) were added, and the two phases were separated. The aqueous solution was extracted with Et₂O (3×50 mL). The combined organic solutions were dried over magnesium sulfate and concentrated. The residue was chromatographed (EtOAc to EtOAc-MeOH 9/1), giving 4.81 g (87%) of the desired product as a colorless oil: ¹H NMR δ 1.21 (s, 3H), 1.56 (dt, J=13, 3 Hz, 2H), 1.65 (td, J=10, 4 Hz, 2H), 2.35 (td, J=10, 3 Hz, 2H), 2.53 (m, 2H), 7.24 (m, 1H), 7.29 (m, 4H); ¹³C NMR δ 30.44, 39.37, 50.39, 63.80, 68.50, 127.56, 128.80, 129.80, 139.17.

1-Benzyl-4-Methyl-4-Phenylpiperidine:

1-Benzyl-4-methyl-piperidin-4-ol (4.81 g, 23.4 mmol) was added to a suspension of AlCl₃ (15.62 g, 117 mmol) in benzene (100 mL) at room temperature under argon. The mixture was stirred at reflux for 24 hours, then cooled and poured cautiously into ice water (100 g of ice, 50 mL of water). The aqueous phase was adjusted to pH 11-12 by addition of 6 N aqueous NaOH at 0° C., and extracted with EtOAc (3×100 mL). The combined organic solutions were dried over magnesium sulfate and concentrated. The residue was chromatographed (hexane-Et₂O 19/1 to 9/1, followed by hexane-EtOAc 3/1), giving the desired product (3.23 g, 52%) as a brown oil: ¹H NMR δ 1.25 (s, 3H), 1.80 (m, 2H), 2.17 (m, 2H), 2.44 (m, 2H), 2.55 (m, 2H), 3.50 (s, 2H), 7.25 (m, 1H), 7.35 (m, 4H); ¹³C NMR δ 36.82, 37.65, 50.95, 54.93, 64.08, 126.19, 126.51, 127.59, 128.83, 128.95, 129.05, 129.89, 139.24.

4-Methyl-4-Phenylpiperidine:

Freshly prepared methanolic formic acid solution (4.4% by weight, 70 mL) was added to 1-benzyl-4-methyl-4-phenylpiperidine (3.23 g, 12.2 mmol). To the resulting solution was added 10% palladium on carbon (2.00 g). The mixture was stirred at room temperature for 24 hours. The solid was filtered out and washed with MeOH (30 mL), H₂O (15 mL), CH₂Cl₂ (30 mL) and MeOH (15 mL). The combined filtrate and washings were concentrated, and the residue was dissolved in CH₂Cl₂ (50 mL) and H₂O (10 mL). The aqueous phase was adjusted to pH 11 by addition of 1 N aqueous NaOH. The organic phase was separated, dried over magnesium sulfate and concentrated. The residual oil was purified by flash chromatography (CHCl₃/MeOH/2 N NH₃ in MeOH 100/4/0 to 100/20/10), giving 1-benzyl-4-methyl-4-phenylpiperidine (1.20 g) and 1.10 g (51%, 82% based on consumed starting material) of 4-methyl-4-phenylpiperidine: ¹H NMR δ 1.24 (s, 3H), 1.71 (m, 2H), 2.06 (m, 2H), 2.82 (m, 3H), 2.94 (m, 2H), 7.19 (m, 1H), 7.32 (m, 4H); ¹³C NMR δ 37.22, 38.54, 43.44, 47.74, 126.31, 127.43, 129.01, 149.73.

3-Aminopropyl-4-Methyl-4-Phenylpiperidine:

A solution of 4-methyl-4-phenylpiperidine (1.00 g, 5.70 mmol), 3-bromo-propylamine hydrobromide (1.87 g, 8.55 mmol) and potassium carbonate (1.97 g, 14.2 mmol) in refluxing dioxane (20 mL) was stirred for 36 hours. After removal of the solvent, water (50 mL) was added and the pH adjusted to 11-12 by the addition of 1 N aqueous NaOH. The mixture was extracted with CH₂Cl₂ (150 mL+3×100 mL). The combined organic solutions were dried over magnesium sulfate and concentrated. The residue was purified by flash chromatography (CHCl₃/MeOH/2 N NH₃ in MeOH 100/20/10), giving the desired product as a colorless oil (241 mg, 18%): ¹H NMR δ 1.18 (s, 3H), 1.61 (p, J=7 Hz, 2H), 1.75 (m, 2H), 2.10 (m, 2H), 2.33 (t, J=7 Hz, 2H), 2.40 (m, 2H), 2.45 (m, 2H), 2.72 (t, J=6 Hz, 2H), 3.02 (br s, 2H), 7.14 (m, 1H), 7.30 (m, 4H); ¹³C NMR δ 30.28, 36.78, 37.64, 41.51, 50.96, 57.51, 126.16, 126.40, 128.91, 149.20.

Preparation of 3-[4-(4-Fluorophenyl)piperidin-1-yl]propylamine

4-(4-Fluorophenyl)Piperidine Hydrochloride:

To a solution of 4-(4-fluorophenyl)-1,2,3,6-tetrahydropyridine hydrochloride (10 g) in methanol (200 mL) was added 10% palladium on charcoal (0.5 g) and the mixture was hydrogenated at 50 psi for 3 h. The catalyst was removed by filtration and solvent was evaporated, leaving the product (10.0 g) as a white powder, which was used in the next step without purification. The product appeared to be pure based on 1H NMR and TLC analysis. ¹H NMR δ 1.95-2.03 (br d, 2H), 2.14-2.29 (m, 2H), 2.70-2.80 (m, 1H), 2.91-3.07 (br q, 2H), 3.60-3.64 (br d, 2H), 6.96-7.03 (m, 2H), 7.19-7.22 (m, 2H), 9.60 (br s, 1H), 9.71 (br s, 1H).

4-(4-Fluorophenyl)Piperidine:

mp ° C.; 1H NMR δ 1.51-1.66 (m, 2H), 1.80 (d, J=7.2 Hz, 2H), 2.53-2.64 (m, 1H), 2.67-2.77 (m, 2H), 3.17 (d, J=12.0 Hz, 2H), 6.94-7.03 (m, 2H), 7.13-7.21 (m, 2H).

Anal. Calcd. For C₁₁H₁₄NF+C₄H₄O₄: C, 58.70; H, 5.83; N, 4.18.

Found: C, 58.72; H, 5.84; N, 3.98.

3-[4-(4-Fluorophenyl)Piperidin-1-yl]Propylphthalimide:

A mixture of 4-(4-fluorophenyl)piperidine hydrochloride (5.08 g, 23.2 mmol), 3-bromopropylphthalimide (6.22 g, 23.2 mmol), and potassium carbonate (15 g) in DMF (100 mL) was stirred at 95-100° C. for 12 h. About 80% of the solvent was evaporated under reduced pressure. The residue was diluted with ethyl acetate (200 mL) and washed with brine (3×100 mL) and dried (Na₂SO₄). The solvent was evaporated from the ethyl acetate solution and the residue was purified by column chromatography (1/1 hexane-ethyl acetate to 100% ethyl acetate), giving crude product (7.50 g, 88%). This crude product was crystallized from isopropanol, giving a white crystalline solid (4.50 g, 1st crop). This material was used in the next step. Concentration of the mother liquor and cooling gave the second crop of desired product (1.0 g). ¹H NMR δ 1.43-1.52 (m, 2H), 1.67-1.75 (m, 2H), 1.80-1.96 (m, 4H), 2.33-2.46 (m, 3H), 2.94-2.99 (br d, 2H), 3.78 (t, J=7 Hz, 2H), 6.90-7.04 (m, 4H), 7.70-7.74 (m, 2H), 7.84-7.87 (m, 2H).

3-[4-(4-Fluorophenyl)Piperidin-1-yl]Propylamine:

Hydrazine (4 mL) was added to a solution of 3-[4-(4-fluorophenyl)piperidin-1-yl]propylphthalimide (4.50 g, 12.3 mmol) in methanol (200 mL), and the mixture was stirred at reflux for 8 h. The solution was cooled to room temperature, and the resulting white solid which formed was filtered and washed with methanol (20 mL). The solvent was evaporated from the filtrate and residue was dried under vacuum for 4 h. The crude product was dissolved in 50 mL of chloroform, stirred for 1 h, and filtered. The white solid was washed with additional chloroform (20 mL), the solvent was evaporated from the combined filtrates to leave the crude product as an oil. The oil was purified by column chromatography (dichloromethane/methanol/2 M ammonia in methanol, 10/3/1), giving the desired product (2.70 g, 93%). ¹H NMR δ 1.60-1.83 (m, GH), 1.96-2.07 (m, 4H), 2.40-2.55 (m, 3H), 2.70-2.85 (br t, 2H), 3.03-3.07 (br d, 2H), 6.93-7.00 (m, 2H), 7.14-7.20 (m, 2H).

4-(4-Methyl-4-(3,5-Dimethylphenyl) Piperidine:

hygroscopic; ¹H NMR δ 1.20 (s, 3H), 1.74-1.80 (m, 2H), 2.08-2.16 (m, 2H), 2.30 (s, 6H), 2.50-2.56 (m, 2H), 2.64-2.68 (m, 2H), 2.97-3.04 (m, 1H), 6.87 (s, 1H), 6.94 (s, 2H).

Benzyl 4-{[(Tert-Butoxycarbonyl)Amino]Methyl) Cyclohexylcarbamate:

Oxalyl chloride (1.1 equivalents) was added dropwise to a mixture of 4-[[(tert-butoxycarbonyl)-amino]methyl] cyclohexanecarboxylic acid (1 equivalent, Maybridge) in toluene. The reaction mixture was stirred at room temperature for 2-6 h. The solvent was removed in vacuo, the residue was dissolved in acetone and the resulting mixture was added dropwise to an aqueous solution of sodium azide (1.2 equivalents) at a rate such as to maintain a temperature of 10-15° C. After the completion of the reaction, the reaction mixture was extracted with ethyl acetate, the combined extracts were dried and concentrated in vacuo. The residue was dissolved in acetone and added slowly to warm (60° C.) benzene. After the completion of the reaction, benzyl alcohol was added to the reaction mixture, stirred for 2 days and the desired product was isolated (For Typical References, See: G. Schroeter Ber. 1909, 42, 3356; and Allen, C.F.H.; Bell, A. Org. Syn. Coll. Vol. 3 (1955) 846.).

A solution of benzyl 4-{[(tert-butoxycarbonyl)amino]methyl}-cyclohexylcarbamate in MeOH containing 10% Pd/C was hydrogenated at 50 psi overnight. The reaction mixture was filtered through Celite 545 and the Celite 545 was washed with methanol. The combined methanol extracts were concentrated in vacuo, giving trans-tert-butyl 4-aminocyclohexylmethylcarbamate (95%).

9H -9-Fluorenylmethyl N-[4-(Aminomethyl)Cyclohexyl]Carbamate:

¹H NMR δ 8.02 (br, 1H), 7.33 (m, 5H), 5.07 (s, 2H), 3.71 (s, 1H), 3.40 (br m, 1H), 2.80 (br m, 2H), 1. 94 (ABq, 4H), 1.68 (br, 1H), 1.30-1.00 (m, 5H).

N1-[4-(Aminomethyl)Cyclohexyl]-1-Naphthamide:

HCl in dioxane (10 mL, 4 N) was added to a solution of tert-butyl[4-(1-naphthoyl-amino)cyclohexyl]methylcarbamate (0.350 g) in dichloromethane (20 mL), stirred overnight, concentrated in vacuo, giving the desired product: ¹H NMR δ 8.24 (dd, 1H, J=1.2, 8.7 Hz), 7.85 (dt, 2H, J=2.7, 9.7Hz), 7.60-7.30 (m, 4H), 5.98 (m, 1H), 4.02 (m, 1H), 3.80-3.40 (m, 4H), 2.53 (d, 2H, J=6.0 Hz), 2.02 (ABq, 4H), 1.41-1.90 (m, 4H).

Tert-Butyl N-(4-[(1-Naphthylcarbonyl)Amino]Cyclohexylmethyl)-Carbamate:

A mixture of 1-naphthoic acid (1.00 mmol, 0.172 9), DMAP (2.00 mmol, 0.250 g) and ECD (0.383 g, 2.00 mmol) in dry dichloromethane (20 mL) was stirred at room temperature for 0.5 h followed by the addition of tert-butyl(4-amino)cyclohexyl)methyl-carbamate amine (1.09 mmol, 0.250 g). The reaction mixture was stirred at room temperature overnight and purified by flash chromatography, giving the desired product as a white solid (0.160 g): ¹H NMR δ 8.29 (dd, 1H, J=1.8, 9.1 Hz), 7.89 (m, 2H), 7.60-7.40 (m, 4H), 5.85 (br d, 1H, J=6.3 Hz), 4.65 (m, 1H), 4.04 (m, 1H), 3.02 (t, 1H, J=6.3 Hz), 2.05 (ABq, 4H), 1.62 (m, 2H), 1.46 (s, 9H), 1.40-1.10 (m, 4H).

4-Acetyl-1-(3-Aminopropyl)-4-Phenylpiperidine:

A solution of 4-Acetyl-4-phenylpiperidine (7, 1.53 g, 7.50 mmol), 3-bromo-propylamine hydrobromide (1.64 g, 7.50 mmol) and potassium carbonate (1.24 g, 9.00 mmol) was stirred in refluxing 1,4-dioxane (50 mL) for 12 h. After removal of dioxane, water (50 mL) was added and the pH was adjusted to 11-12 by addition of 1 N aqueous NaOH. The mixture was extracted with CH₂Cl₂ (100 mL+3×50 mL). The combined organic solutions were dried over magnesium sulfate and concentrated. The residue was purified by flash chromatography (EtOAc-MeOH-Et3N 100/40/20), giving the desired product as a colorless oil (780 mg, 40%): ¹H NMR δ 1.56 (p, J 7 Hz, 2H), 1.84 (s, 3H), 1.98 (m, 2H), 2.15 (br t, J=12 Hz, 2H), 2.29 (t, J=7 Hz, 2H), 2.41 (br d, J=12 Hz, 2H), 2.66 (t, J=7 Hz, 4H), 7.18-7.30 (m, 5H); ¹³C NMR δ 26.28, 31.11, 33.43, 41.47, 51.62, 55.31, 57.19, 77.32, 77.74, 78.17, 126.95, 127.69, 129.44, 142.25, 210.15.

For the preparation of benzo-4′,5′[H]furanpiperidine refer to W. E.Parham et al, J. Org. Chem. (1976) 41, 2268.

Tert-Butoxy{[3-(Benzo-4′,5′[H]Furanpiperidin-1-yl)Propyl]Amino}Methanol:

To a stirred solution of the N-[4-(benzo-4′,5′[H]furanpiperidine (0.566 g, 3.27 mmol) in dioxane (20 mL) N-(tert-butoxycarbonyl)-3-bromopropylamine (0.772 g, 3.27 mmol) and potassium carbonate (0.904 g, 6.54 mmol) were added and the solution was refluxed for 24 h. The reaction mixture was cooled to room temperature, concentrated and partitioned between chloroform (40 mL) and water (5 mL). The organic layer was dried over sodium sulfate, filtered and concentrated. The crude product was purified by column chromatography (ethyl acetate/methanol, 4.5/0.5), giving the desired product as a colorless oil (0.856 g, 79%); ¹H NMR (1.45 (s, 9H), 1.63-2.04 (m, 6H), 2.33-2.52 (m, 4H), 2.87 (d, J=l1.0 Hz, 2H), 3.2 (br s, 2H), 5.07 (s, 2H), 5.6 (br s, 1H), 7.13-7.28 (m, 4H).

3-(4-Methyl-4-Phenyl-1-Piperdinyl)Propylamine:

Trifluoroacetic acid (1 mL) was added to tert-butoxy{[3-(4-methyl-4-phenyl-1-piperdinyl)propyl]-amino}methanol(0.500 g, 1.51 mmol) in dichloromethane (5 mL) and the solution was stirred at room temperature for 1 h. The solution was concentrated, neutralized with 10 % KOH solution and extracted with dichloromethane (25 mL). The organic layer was dried over sodium sulfate, filtered and concentrated, giving 0.340 g (98%) of 3-(4-methyl-4-phenyl-1-piperdinyl)propylamine which was used without further purification in the subsequent step.

Procedures for the Reaction of the Amine Side Chains with the p-Nitrophenylcarbamate Intermediates:

General Procedure:

An equimolar solution of an amine side chain such as 3-(4-methyl-4-phenyl-1-piperdinyl)propylamine and a p-nitrophenylcarbamate intermediate such as 5-methoxycarbonyl -4-methoxymethyl-1,2,3,6-tetrahydro-2-oxo-6-(3,4-difluorophenyl)-1-[(4-ni trophen-yloxy)carbonyl]pyrimidine and 1-2 equivalents of a base such as diisopropylethylamine in dichloromethane were stirred at room temperature overnight. The reaction mixture was concentrated and purified by flash chromatography, giving the desired product. In case of 2-methoxy intermediates, conversion to the oxo derivatives was accomplished by treatment of the 2-methoxy product with HCl in dioxane.

2-oxo-3-{SPIRO[1H-INDANE-1,4′-PIPERIDINE]PROPYLAMINE(0.0 319 g, 0.123 mmol) was added to (±)-6-(3,4-difluoro-phenyl)-1,6-dihydro-2-methoxy-5-methoxycarbonyl-4-ethyl-1-(4-nitrophenoxy)carbonyl-pyrimidine (0.052 g, 0.112 mmol) in dry dichloromethane (10 mL) and the solution was stirred at room temperature for 24 h. The reaction mixture was stirred for another 1 h after addition of 6 N HCl (2 mL). After neutralization with aqueous 10% KOH solution, the reaction mixture was extracted into dichloromethane (3×10 mL). The organic layer was dried over sodium sulfate, filtered and concentrated. The crude product was purified by flash chromatography (EtOAc/ MeOH, 4.5/0.5), giving of the desired product (0.040 g) as a syrup.

1 N HCl in ether (5 mL) was added to the free base (0.040 g, 0.072 mmol) in dichloromethane (4 mL) and the solution was concentrated under reduced pressure. The crude product was recrystallized from ether, giving the desired compound (0.042 g, 99%) as a pale yellow solid; mp 178-182° C.; Anal. Calcd. for C₂₉H₃₄F₂N₄O₅Cl₂+0.6 H₂O: C, 57.87; H,5.73, N 9.31. Found: C, 58.11; H 5.90; N 8.95.

General Procedure for the reaction of the piperidines and piperazines with 1-(3-bromo-propylcarbamoyl) -6-(3,4-difluoro-phenyl)-4-methyl-2-oxo-1,6-dihydro-pyrimidine-5-carboxylic acid methyl ester:

The amine (0.15 mmol) was added to a solution of 1-(3-bromo-propylcarbamoyl)-6-(3,4-difluorophenyl)-4-methyl-2-oxo-1 ,6-di-hydropyrimidine-5-carboxylic acid methyl ester (43.0 mg, 0.100 mmol) in anhydrous acetone (10 mL), followed by NaHCO₃ (41 mg, 0.3 mmol) and KI (16 mg, 0.1 mmol). The resulting suspension was heated to reflux for 10 h and then cooled to room temperature. The solvent was removed in vacuo and the residue was purified by flash column chromatography (EtOAc, followed by EtOAc/MeOH, 9/1). The product was then dissolved in 2 mL of chloroform, acetone or EtOAc and HCl in Et₂O (1 M, 0.5 mL) was added at room temperature. The solvent was removed in vacuo, giving the desired compound as an HCl salt.

### EXAMPLE 1

(−)-1,2,3,6-Tetrahydro-1-{N-[4-(3,-Acetamido)-Phenyl-piper idin-1-yl]Propyl}Carboxamido-4-Methoxymethyl-6-(3,4-Difluoro-Phenyl)-2-Oxopyrimidine-5-Carboxylic Acid Methyl Ester:

ESMS, 612.25 (M+1); ¹H NMR δ 1.76-1.87 (m, 6H), 2.03-2.13 (m, 2H), 2.18 (s, 3H), 2.49 (t, J=6.9 Hz, 3H), 3.10 (d, J=11.1 Hz, 2H), 3.30-3.42 (m, 2H), 3.45 (s, 3H), 3.71 (s, 3H), 4.68 (s, 2H), 6.68 (s, 1H), 6.96 (d, J=7.5 Hz, 1H), 7.04-7.11 (m, 2H), 7.16-7.26 (m, 2H), 7.34 (d, J=6.3 Hz, 1H), 7.45 (s, 1H), 7.94 (s, 1H), 8.98 (t, J=5.4 Hz, 1H).

### EXAMPLE 2

Methyl 3-[(3-4-[3-(Acetylamino)Phenyl]-1,2,3,6-Tetrahydro-1-Pyridinylpropyl)Amino]Carbonyl-4-(3,4-Difluorophenyl)-6-(Me thoxy-Methyl)-2-oxo-1,2,3,4-Tetrahydro-5-Pyrimidine-Carboxylate:

¹H NMR δ 8.90 (t, 1H, J=3.6 Hz), 7.75 (s, 1H), 7.50-7.00 (m, 8H), 6.68 (s, 1H), 6.03 (br s, 1H), 4.67 (s, 2H), 3.71 (s, 3H), 3.47 (s, 3H), 3.38 (ABm, 2H), 3.16 (m, 2H), 2.71 (t, 2H, J=5.4 Hz), 2.56 (m, 4H), 2.35-1.90 (br, 2H), 2.17 (s, 3H), 1.82 (p, 2H, J=7.2 Hz); ESMS, 612.25 (M+1).

### EXAMPLE 3

(1)-1,2,3,6-Tetrahydro-1-{N-[3-(4-O-Acetyl)-4-Phenylpipe Ridin-1-yl]Propyl)Carboxamido-5-Methoxycarbonyl-4-Methoxymethyl-6-(3,4-Difluorophenyl)-2-Oxopyrimidine: 4-Acetyl-1-(3-aminopropyl)-4-phenylpiperidine (190 mg, 0.687 mmol) was added to a stirring solution of 5-methoxycarbonyl-4-methoxymethyl-1,2,3,6-tetra-hydro-2-oxo-6-(3,4-difluorophenyl)-1-[(4-nitrophenyloxy) carbon-yl]pyrimidine (281 mg, 0.573 mmol) in dry dichloromethane (3 mL) and THF (4 mL). The reaction mixture was stirred at room temperature for 12 h. The reaction mixture was quenched with aqueous 6 N HCl. The reaction mixture was concentrated to a small volume, partitioned between dichloromethane and water (100 mL each), the mixture was adjusted to pH 8 by addition of Na₂CO₃, the layers were separated, and the aqueous layer was extracted with dichloromethane (3×30 mL). The combined organic extracts were dried (Na₂SO₄) and the product was chromatographed, giving the desired product. The HCl salt was prepared by the addition of 1 N HCl in ether to a solution of the product in CH₂Cl₂. The precipitated salt was filtered, washed with ether and dried in vacuo, giving (1)-1,2,3,6-tetrahydro-1-{N-[3-(4-0-acetyl)-4-phenylpiperidin-1-yl]propyl}carboxamido-5-methoxycarbony 1-4-methoxymethyl-6-(3,4-difluorophenyl)-2-oxopyrimidine (170 mg, 47%) as the hydrochloride salt: (C₃₁H₃₆N₄F₂O₇+HCl+0.6 CH₂Cl₂); mp 82-84° C.

### EXAMPLE 4

Benzyl ester precursor to the product of Example 4:

(+)-1,2,3,6-Tetrahydro-1-{N-[4-(Benzo-4′,5′(H)Furan)Pipe ridin-1-yl]Propyl}-Carboxamido-4-Ethyl-6-(3,4-Difluorophenyl)-2-oxo-Pyrimidine-5-Carboxylic Acid Phenylmethyl Ester:

¹H NMR δ 7.60-7.00 (m, 12H), 6.85 (br, 1H), 6.62 (s, 1H), 5.10 (ABq, 2H), 5.67 (s, 2H), 4.03 (br, 1H), 4.01 (s, 3H), 3.40 (apparent q, 2H, J=6.8 Hz), 3.20-1.60 (m, 12H), 2.86 (q, 2H, J=2.5 Hz), 1.19 (t, 3H, J=7.5 Hz).

(+)-1,2,3,6-Tetrahydro-1-{N-[4-(Benzo-4′,5′(H)Furan)Pipe ridin-1-yl]Propyl }-Carboxamido-4-Ethyl-6-(3,4-Difluorophenyl)-2-oxo-Pyrimidine-5 Carboxylic Acid Hydrochloride:

¹H NMR δ 8.95 (br s, 1H), 8.22 (br s, 1H), 7.40-6.95 (m, 7H), 6.95 (s, 1H), 6.63 (s, 1H), 5.10-4.95 (m, 2H), 3.40-3.20 (m, 4H), 3.10-2.80 (m, 4H), 2.55-2.20 (m, 1H), 2.15 (m, 1H), 1.85 (m, 2H), 1.55-1.30 (m, 4H), 1.20 (t, 3H, J=7.6 Hz); Anal. Calc. For C₂₉H₃₂N₄O₅F₂+HCl+1.5 H₂O: C, 56.36; H, 5.87; N, 8.06. Found: C, 56.72; H, 6.11; N, 7.61.

### EXAMPLE 5

1,2,3,4-Tetrahydro-1-oxo-2-Naphthacetic Acid Methyl Ester:

Under argon, α-tetralone (5.00 g, 34.2 mmol) in dry THF (300 mL) was treated with LDA in THF (2 M, 18.8 mL) at −78° C. The solution was stirred at −78° C. for 1 h. Methyl bromoacetate (15.7 g, 0.103 mole) was then added to the solution, the mixture was stirred overnight and allowed to warm to room temperature. The solvent was evaporated and the residue was dissolved into CHCl₃ (300 mL), washed with water and saturated brine, and then dried over Na₂SO₄. After filtration and removal of solvent, the residue was vacuum distilled. The product, a colorless oil (7.21 g, 96.5%) was collected at 180° C./1 mm Hg; ¹H NMR (400 Mhz) δ 1.98 (m, 1H), 2.25 (m, 1H), 2.44 (m, 1H), 2.90-3.20 (m, 4H), 3.73 (s, 3H), 7.10-8.10 (m, 4H); EI mass spectrum M+ at m/z 218.

1-Hydroxy-2-(2-Hydroxyethyl)-1,2,3,4-Tetrahydronaphthale ne:

A solution of 1,2,3,4-tetrahydro-1-oxo-naphthacetic acid methyl ester (6.15 g, 28.2 mmol) in THF (150 mL) was treated with LiAlH₄ (2.82 g, 70.5 mmol) and then the reaction mixture was heated at reflux temperature for 5 h. The suspension was cooled to 0° C. and quenched by addition of solid Na₂SO₄.10 H₂O. The mixture was stirred at room temperature for 4 hrs. The solid was removed by filtration and concentration of the filtrate in vacuo gave a yellow oil (5.33 g, 98.3%); ¹H NMR indicated the formation of an isomeric mixture. EI mass spectrum M+ at m/z 192. The mixture was directly used in next reaction without further purification.

2-(2-Hydroxyethyl)-1,2,3,4-Tetrahydro-1-oxo-Naphthalene:

A solution of isomeric mixture of 1-hydroxy 1-2-(2-hydroxyethyl)-1,2,3,4-tetrahydronaphthalene (3.00 g, 15.6 mmol) in CH₂Cl₂ (100 mL) was treated with MnO₂ (20.4 g, 0.234 mole). The suspension was stirred at room temperature for 16 h and the solids were removed by filtration. Concentration of the filtrate in vacuo gave a brown oil, which was further purified by flash chromatography (MeOH/ CHCl₃, 5/95), giving a yellow oil (2.00 g, 67.4%) ¹H NMR δ 1.76 (m, 1H), 1.98 (m, 1H), 2.21 (m, 2H), 2.57 (br, 1H), 2.70 (m, 2H), 3.20 (m, 2H), 3.81 (m, 2H) 7.00-8.20 (m, 4H); CI mass spectrum (M+1)+ at m/z 191.

2-(2-Bromoethyl)-1,2,3,4-Tetrahydro-1-Oxonaphthalene:

A solution of 2-(2-hydroxethyl)-1,2,3,4-tetrahydro-1-oxo-naphthalene (2.00 g, 10.5 mmol) in CH₂Cl₂ (100 mL) was treated with PBr₃ (948 mg, 3.50 mmol) at 0° C. The mixture was stirred at room temperature for 72 h and then poured onto 100 g of ice. The organic layer was separated, washed with aqueous 10% K₂CO₃ solution, H₂O, saturated NaCl and dried over Na₂SO₄. After filtration and removal of the solvent, the residue was purified by chromatography (EtOAc/hexane, 1/10), giving a yellow oil (1.18 g, 44.4%); ¹H NMR δ 1.49 (m, 2H), 2.24 (m, 1H), 2.60 (m, 1H), 2.75 (m, 1H), 3.03 (m, 2H), 3.64 (m, 2H), 7.10-8.10 (m, 4H); EIMS M+ m/z 223, M/M+2=1:1.

2-[2-(4-Benzamino-1-Piperidyl)Ethyl]-1,2,3,4-Tetrahydro-1-oxo-Naphthalene:

A mixture of 2-(2-bromoethyl)-1,2,3,4-tetrahydro-1-oxonaphthalene (1.18 g, 4.66 mmol), 4-benzamidopiperidine (952 mg, 4.66 mmol) and K₂CO₃ (1.29 g, 9.32 mmol) in acetone (200 mL) was stirred at room temperature for 48 h. The solids were removed by filtration. Concentration of filtrate in vacuo gave a yellow solid which was purified by chromatography (MeOH: CHCl₃, 5/95). The product was recrystallized from an EtOAc/hexane mixture, giving a white powder (268 mg, 15.3%); mp 158-159° C.; ¹H NMR δ 1.53 (m, 2H), 1.67 (m, 1H) 1.91 (m, 1H), 2.02 (m, 2H), 2.21 (m, 4H), 2.50 (m, 3H), 2.95 (m, 4H), 4.01 (m, 1H), 5.95 (d, J=8.0 Hz, 1H), 7.20-8.10 (m, 9H); CI MS (M+1) +m/z 377; Anal. Calcd for C₂₄H₂₈N₂O₂: C, 76.55; H. 7.51; N, 7.44. Found: C, 76.28; H, 7.46; N, 7.37.

### EXAMPLE 6

Methyl 4-(2,1,3-Benzoxadiazol-5-yl)-3-[(1-(4-(Dibutylamino)-Benzyl]-4-Piperidylmethyl)Amino]Carbonyl-6-Methyl-2-oxo-1,2,3,4-Tetrahydro-5-Pyrimidinecarboxylate:

¹H NMR δ 7.72 (dd, 1H, J=0.6, 9.6 Hz), 7.70-7.50 (m, 2H), 7.11 (d, 2H, J=8.7 Hz), 6.59 (d, 2H, J=8.7 Hz), 5.90 (s, 1H), 3.94 (s, 3H), 3.63 (s, 2h), 3.24 (t, 4H, J=7.8 Hz), 2.80 (m, 2H), 2.49 (d, 2H, J=6.3 Hz), 2.38 (s, 3H), 2.90-1.00 (m, 5H), 1.54 (p, 4H, J=7.8 Hz), 1.35 (sextet, 4H, J=7.8 Hz), 0.94 (t, 6H, J=7.8 Hz).

### EXAMPLE 7

(+)-1,2,3,6-Tetrahydro-1-{N-[4-(N′-Ethyl)-N-Benzimidazol yl Piperidin-1yl]Propyl}Carboxamido-4-Methyl-6-(3,4-Difluor ophenyl)-2-Oxopyrimidine Hydrochloride:

¹H NMR δ 8.95 (t, 1H, J=3.6 Hz), 7.61 (b, 1H), 7.60-6.95 (m, 7H), 6.69 (s, 1H), 4.36 (m, 1H), 3.94 (q, 2H, J=7.2 Hz), 3.72 (s, 3H), 3.42 (ABm, 4H), 3.30 (m , 2H, 4.76 (m, 4H), 2.43 (s, 3H), 2.13 (m, 2H), 1.77 (m, 4H), 1.33 (t, 3H, J=7.2 Hz)

### EXAMPLE 8

6-(Benzofurazan-5-yl)-1,2,3,6-Tetrahydro-5-Methoxycarbon yl-4-Methyl-2-oxo-1-{N-[3-(4-Phenylpiperidin-1-yl) Propyl]}Carboxamido-Pyrimidine:

A solution of 6-(benzo-furazan-5-yl)-1,6-dihydro-2-methoxy-5-methoxycarbonyl-4-methyl-1-{N-[3-(4-phenylpipe ridin-1-yl)propyl]}carboxamidopyrimidine in MeOH was treated with 6 N HCl at 0° C. The solution was stirred at room temperature for 2 h and the MeOH was removed in vacuo. 6-(Benzofurazan-5-yl)-1,2,3,6-tetrahydro-5-methoxycarbonyl -4-methyl-2-oxo-1-{N-[3-(4-phenylpiperidin-1-yl)propyl]}carboxamidopyrimidine hydrochloride was obtained as a white powder: mp 134-137° C.

### EXAMPLE 9

4-(3-Methoxy)-Phenyl Piperidine:

HCl salt; mp 150-154° C.; ¹H NMR δ 2.04 (s, br, 2H), 2.25 (s, br, 2H), 2.80 (s, br, 1H), 3.09 (s, br, 2H), 3.66 (s, 2H), 3.78 (s, 3H), 6.79 (s, br, 3H), 7.23 (s, 1H), 9.41 (s, br, 1H). Anal. Calcd. For C₁₂H₁₈NOCl+0.30 CH₂Cl₂: C, 58.34; H, 7.40; N, 5.53. Found: C, 58.30; H, 7.71; N, 5.35.

(+)-1,2,3,6-Tetrahydro-1-N-[4-(3-Methoxy)-Phenyl}-Piperi din-1-yl]-Propyl-Carboxamido-4-Methoxymethyl-6-(3,4-Difluorophenyl)-2-Oxopyrimidine-5-Carboxylic Acid Methyl Ester:

mp 80-84° C.; [α]_{D}=+94.7, (c=0.25, MeOH); ¹H NMR δ 1.74-1.84 (m, 6H), 1.99-2.09 (m, 2H), 2.38-2.51 (m, 3H), 3.03 (d, J=11.1 Hz, 2H), 3.24-3.43 (m, 2H), 3.48 (s, 3H), 3.71 (s, 3H), 3.80 (s, 3H), 4.72 (s, 2H), 6.68 (s, 1H), 6.72-6.84 (m, 3H), 7.05-7.11 (m, 2H), 7.15-7.27 (m, 2H), 7.72 (s, 1H), 8.84 (t, J=5.4 Hz, 1H). Anal. Calcd. For C₃₀H₃₇N₄O₆F₂Cl: C, 57.8; H, 6.0; N, 9.0. Found: C, 57.61; H, 6.57; N, 6.97.

### EXAMPLE 10

(+)-1,2,3,6-Tetrahydro-1-(N-[4-(3,-Acetamido)-Phenyl-Pip eridin-1-yl]Propyl}Carboxamido-4-Methoxymethyl-6-(3,4-Di fluoro-Phenyl)-2-Oxopyrimidine-5-Carboxylic Acid Methyl Ester:

mp 135-138° C.; [α]_{D}=+105.5, (c=0.11, MeOH); ESMS, 614.25 (M+1); ¹H NMR δ 1.76-1.87 (m, 6H), 2.03-2.13 (m, 2H), 2.18 (s, 3H), 2.49 (t, J=6.9 Hz, 3H), 3.10 (d, J=11.1 Hz, 2H), 3.30-3.42 (m, 2H), 3.46 (s, 3H), 3.71 (s, 3H), 4.68 (s, 2H), 6.68 (s, 1H), 6.96 (d, J=7.5 Hz, 1H), 7.04-7.11 (m, 2H), 7.16-7.26 (m, 2H), 7.34 (d, J=6.3 Hz, 1H), 7.45 (s, 1H), 7.94 (s, 1H), 8.97 (t, J=5.4 Hz, 1H); ESMS, M+1 614.25

The compound of Example 10 may also be prepared via hydrogenation of the compoun of example 2 (H₂ balloon method, methanol, Pd/C, overnight). A synthetic path analogous to the latter route (Scheme 11) was used in the preparation of the tritiated analog, which in turn, was used as a radioligand in the MCH pharmacological assays.

### EXAMPLE 11

3-(4-Phenylpiperidin-1-yl)Propionitrile:

Acrylonitrile (3.1 mL, 44 mmol, 2.5 eq) was added to a solution of 4-phenylpiperidine (3.00 g, 18.0 mmol) in EtOH (40 mL) and the mixture was stirred at room temperature for 1.5 h. The volatiles were removed, giving 3.80 g of the desired product (brown oil, 99%).

3-(4-Phenylpiperidin-1-yl) Propylamine:

A solution of BH₃ in THF (1.0 M, 83.0 mL, 83.0 mmol, 3.5 eq) was added to a stirring solution of 3-(4-phenylpiperidin-1-yl)-propionitrile (5.10 g, 24.0 mmol) in anhydrous THF (20 mL) under argon at room temperature. The mixture was heated at reflux temperature for 4.5 hours and then cooled to room temperature. Aqueous 6 N HCl (130 mL) was added and stirring was continued for 2 hours at 50-70° C. The mixture was basified to pH 9 by addition of aqueous 6 N NaOH and extracted with EtOAc (100 mL) and CH₂Cl₂ (3×100 mL). The combined organic extracts were dried over magnesium sulfate and concentrated. The residue was dissolved in CH₂Cl₂ (20 mL) and treated with HCl in ether (1.0 M, 50 mL). The solvents were removed, ether (250 mL) was added, the mixture was filtered, and the filter cake was washed with ether. Water (60 mL) was added to the resulting white solid, 1 N NaOH was added until pH 10-11 was reached, and then the aqueous phase was extracted with CH₂Cl₂ (3×50 mL). The combined extracts were dried over magnesium sulfate and the solvents were evaporated, giving the desired product (4.50 g, 87%).

6-(3,4-Diflourophenyl)-1,2,3,6-Tetrahydro-5-Methoxycarbo nyl-4-Methyl-2-oxo-1-{N-[3-(4-Phenylpiperidin-1-yl) propyl]}Carboxamido-Pyrimidine:

A solution of 6-(3,4-difluorophenyl)-1,6-dihydro-2-methoxy-5-methoxy carbonyl-4-methyl-1-{N-[3-(4-phenyl-piperidin-1-yl)propyl]}carboxamidopyrimidine (100 mg, 0.185 mmol, mp=43-45° C.) in MeOH (5 mL) was treated with aqueous 6 N HCl (1. 5 mL) at 0° C. The solution was stirred at room temperature for 2 hrs and MeOH was removed in vacuo. 6-(3,4-Diflourophenyl)-1,2,3,6-tetrahydro-5-methoxycarbonyl-4-methyl-2-oxo-1-{N-[3-(4-phenylpiperidin-1-yl)propyl]}carboxamidopyrimidine hydrochloride was obtained as a white powder (89 mg, 86%). mp 133-136° C.

### EXAMPLE 12

3-{(3,4,5-Trifluorophenyl)Methylene}-2,4-Pentanedione:

A stirring mixture of 3,4,5-trifluorobenzaldehyde (4.2 g, 26.2 mmol), 2,4-pentanedione (2.62 g, 26.2 mmol), piperidine (0.430 g, 5 mmol) in benzene (150 mL) was heated at reflux temperature (equipped with a Dean-Stark trap) for 8 h. The benzene was evaporated, the yellow oily residue, 2-{(3,4,5-trifluorophenyl)-methylene}-2,4-pentanedione, was used in the next step without further purification.

6-(3,4,5-Trifluorophenyl)-1,6-Dihydro-2-Methoxy-5-Acetyl -4-Methylpyrimidine:

A stirring mixture of 2-{(3,4,5-trifluoro-phenyl)methylene}-2,4-pentanedione (26.2 mmol), O-methylisourea hydrogen sulfate (3.22 g, 39.3 mmol), and NaHCO₃ (6.60 g, 78.6 mmol) in EtOH (400 mL) was heated at 95-100° C. for 6 h. The mixture was filtered, the solid residue was washed with ethanol (100 mL). The solvent was evaporated from the combined filtrates and the crude product was purified by flash column chromatography (EtOAc/hexane, 9/1 to 4/1), giving the desired product as an oil (2.80 g, 36%).

6-(3,4,5-Trifluorophenyl)-1,6-Dihydro-2-Methoxy-5-Acetyl -4-Methyl-1-[(4-Nitrophenyloxy)Carbonyl]Pyrimidine:

4-Nitrophenyl chloroformate (1.886 g, 9.38 mmol) was added to a solution of 6-(3,4,5-trifluorophenyl)-1,6-dihydro-2-methoxy-5-acetyl-4-methylpyrimidine (2.80 g, 9.38 mmol) and pyridine (10 mL) in CH₂Cl₂ (200 mL) at 0-5° C. and then the mixture was allowed to warm to room temperature. After 12 h, the solvent was evaporated and the residue was purified by flash chromatography (CH₂Cl₂/EtOAc, 9/1 to 20/3), giving the desired product as a white powder (4.0 g, 92%).

6-(3,4,5-Trifluorophenyl)-1,2,3,6-Tetrahydro-2-oxo-5-Ace tyl-4-Methyl-1-[(4-Nitrophenyloxy)Carbonyl]Pyrimidine:

Aqueous 6 N aqueous HCl (4 mL) was added to a stirring solution of 6-(3,4,5-trifluorophenyl)-1,6-dihydro-2-methoxy-5-acetyl-4-methyl-1-[(4-nitrophenyloxy)carbonyl]pyrimidine (4.0 g, 8.63 mmol) in THF (100 mL) at 0-5° C., and the mixture was allowed to warm to room temperature. After 2 h, the solvent was evaporated and the product was dried under vacuum, giving the desired product as a pure single component which was used in the next step without further purification (3.88 g, 100%).

(+)-1,2,3,6-Tetra Hydro-1-{N-[4-(4-Fluorophenyl)-Piperidine-1-yl]-Propyl}Carboxamido-5-Acetyl-2-oxo-6-(3,4 ,5-Tri Fluoro Phenyl)-4-Methyl Pyrimidine Hydrochloride:

¹H NMR δ 7.20-6.86 (m, 6H), 6.64 (s, 1H), 5.56 (s, 1H), 3.70-3.80 (m, 2H), 3.43-3.35 (m, 2H), 3.19-2.98 (m, 2H), 2.40 (s, 3H), 2.28 (s, 3H), 2.50-1.60 (m, 8H).

### EXAMPLE 13

N1-[4-([4-(Dibutylamino)Benzyl]Aminomethyl)Cyclohexyl]-1-Naphth-Amide:

¹H NMR δ 8.26 (dd, 1H, J=2.1, 7.2 Hz), 7.87 (m, 2H), 7.51 (m, 2H), 7.40 (apparent t, 1H, J=7.8 Hz), 7.17 (d, 1H, J=8.7 Hz), 6.61 (d, 2H, J=8.7 Hz), 5.94 (d, 1H, J=8,1 Hz), 4.04 (m, 1H), 3.76 (m, 1H), 3.63 (m, 2H), 3.21 (t, 4H, J=7.6 Hz average), 2.53 (d, 2H, J=6.7 Hz), 2.10, ABm, 4H), 1.55 (p, 4H, J=7.7 Hz average), 1.34 (sept, 4H, J=7.6 Hz average), 1.17 (m, 4H), 0.95 (t, 6H, J=7.6 Hz average).

### EXAMPLE 14

(+)-1,2,3,6-Tetrahydro-1-{N-[4-(1-Naphthyl)-Piperidin-1-yl]Prop-yl}Carboxamido-4-Methoxymethyl-6-(3,4-Difluorophenyl) -2-oxo-Pyrimidine-5-Carboxylic Acid Methyl Ester:

mp 168-172° C.; [α]_{D}=+94.7, (c=0.25, MeOH); ¹H NMR δ 1.75-1.84 (m, 2H), 1.87-2.01 (m, 4H), 2.14-2.28 (m, 2H), 2.47 (t, J=7.2 Hz, 2H), 3.10 (d, J=11.1 Hz, 2H), 3.28-3.45 (m, 3H), 3.48 (s, 3H), 3.71 (s, 3H), 4.68 (s, 2H), 6.70 (s, 1H), 7.05-7.12 (m, 2H), 7.16-7.24 (m, 1H), 7.42-7.54 (m, 4H), 7.69-7.75 (m, 2H), 7.85 (d, J=11.4 Hz, 1H), 8.09 (d, J=11.1 Hz, 1H), 8.91 (t, J=5.4 Hz, 1H).

### EXAMPLE 15

4-(5-Fluoro-2-Methoxy)Phenyl Piperidine:

mp 254-258° C.; ¹H NMR δ 1.53-1.68 (m, 2H), 1.79 (d, J=11.7 Hz, 2H), 2.12 (dt, J=2.1 Hz, J=11.7 Hz, 1H), 2.77 (dt, J=1.8 Hz, J=12.3 Hz, 1H), 2.90-3.05 (m, 1H), 3.10-3.22 (m, 2H), 3.68 (s, 1H), 3.79 (s, 3H), 6.72-6.93 (m, 3H). Anal. Calcd. For C₁₂H₁₇NOFCl+0.14 CH₂Cl₂: C, 56.60; H, 6.76; N, 5.44. Found: C, 56.60; H, 6.92; N, 5.28.

(+)-1,2,3,6-Tetrahydro-1-{N-[4-(5-Fluoro-2-Methoxy)Pheny lpiperi-din-1-yl]Propyl}Carboxamido-4-Methoxymethyl-6-(3,4-Difluoro-phenyl)-2-Oxopyrimidine-5-Carboxylic Acid Methyl Ester:

¹H NMR δ 8.93 (t, 1H, J=5.4 Hz), 7.76 (br, 1H), 7.30-6.69 (m, 7H), 4.69 (s, 2H), 3.79 (s, 3H), 3.71 (s, 3H), 3.48 (s, 3H), 3.38 (m, 2H), 3.10-2.80 (m, 3H), 2.42 (t, 2H, J=7.2 Hz), 2.07 (dt, 2H, J=3.0, 8.4 Hz), 2.00-1.60 (m, 6H).

### EXAMPLE 16

(+)-1,2,3,6-Tetrahydro-1-{N-[4-Hydroxy-4-(2-Pyridyl)-pip eridin-1-yl]Propyl}Carboxamido-4-Methoxymethyl-6-(3,4-Difluorophenyl)-2-Oxopyrimidine-5-Carboxylic Acid Methyl Ester:

mp 132-135° C.; [α]_{D}=+94.7, (c=0.25, MeOH); ¹H NMR δ 1.47 (d, J=11.7 Hz, 2H), 1.74-1.85 (m, 2H), 2.43-2.63 (m, 9H), 2.87 (d, J=10.2 Hz, 2H), 3.30-3.47 (m, 2H), 3.49 (s, 3H), 3.71 (s, 3H), 4.69 (s, 2H), 6.69 (s, 1H), 7.04-7.21 (m, 4H), 7.49 (dd, J=0.6 Hz, J=6.9 Hz, 1H), 7.72 (s, br, 1H), 8.36 (dd, J=1.2, 4.8 Hz, 1H), 8.89 (t, J=5.4 Hz, 1H)

### EXAMPLE 17

1-(3-Aminopropyl)-4-[2-Pyridyl]Pyridinium Bromide Hydrobromide:

A solution of 2,4¹-dipyridyl (25.0 g, 160 mmol) and 3-bromopropyl-amine hydrobromide (35.0 g, 160 mmol) in DMF (60 mL) was heated at 90-95° C. for 10 h. After cooling to room temperature, anhydrous ether (500 mL) was added to the mixture, the resulting white solid was filtered, washed with Et₂O and dried, giving 1-(3-aminopropyl)-4-[2-pyridyl]pyridinium bromide hydrobromide (60 g, 100%)). ¹H NMR (DMSO-d₆) 62.35-2.44 (m, 2H), 3.08-3.13 (m, 2H), 4.76-4.81 (m, 2H), 7.58 (dd, J=4.8 Hz, J=7.5 Hz, 1H), 8.03 (dt, J=1.8 Hz, J=7.8 Hz, 1H), 8.32 (d, J=7.8 Hz, 1H), 8.77-8.81 (m, 3H), 9.12 (d, J=6.3 Hz, 2H). Anal. Calcd. for C₁₃H₁₆N₃Br+HBr+0.5 H₂O: C, 40.65; H, 4.72; N, 10.94. Found: C, 40.83; H, 4.37; N, 11.05.

3-(3′,6′-DIHYDRO-2′-H-[2,4′] Bipyridinyl-1′-yl)-Propylami ne:

NaBH₄ (2 g, 53 mmol) in small portions was added to a solution of 1-(3-aminopropyl)-4-[2-pyridyl]pyridinium bromide hydrobromide (6 g, 16 mmol) in MeOH (150 mL) at 0-5° C. over a period of 2 h. The reaction mixture was stirred overnight at room temperature and then the solvent was evaporated. The residue was suspended in ether (200 mL) and treated with aqueous 50% NaOH solution (100 mL). The ether layer was separated and the aqueous layer was extracted with additional ether (2×50 mL). The combined ether extracts were dried over potassium carbonate and the solvent was removed, giving 3-(3′,6′-dihydro-2′-H-[2,4′]bipyridinyl-1′-yl)- propylamine (3.48 g) as an oil. The crude product was used in the next step immediately without further purification.

3-Aminopropyl-4-(2-Pyridyl)Piperidine:

A suspension of 3-(3′,6′-dihydro-2′-H-[2,4′]bipyridinyl-1′-yl)-propylamine (3.48 g crude, 15.9 mmol) and Pearlman's catalyst (1.0 g) in MeOH (40 mL) was hydrogenated under 120 psi for 10 h, after which the reaction mixture was filtered through a pad of Celite and the solvent was removed. The residue was purified by column chromatography over silica gel (30 g) [Note: If a large excess of silica gel is used the recovery of the product will be very low] (CH₂Cl₂/methanol/2M NH3 in MeOH, 90/8/4 to 90/40/40). The product was obtained as a pale yellow oil (3.21 g, 91%). ¹H NMR δ (CD₃OD) 1.50-1.99 (m, 10H), 2.02-2.06 (m, 2H), 2.37-2.75 (m, 3H), 3.02-3.06 (br m, 2H), 7.05-7.09 (m, 4H), 7.16 (dt, J=0.9 Hz, J=8.7 Hz, 1H), 8.48 (dd, J=0.9 Hz, J=4.2 Hz, 1H).

Part II

(+)-6-(3,4-Difluorophenyl)-1-{N-[4-(2-Pyridyl)Piperidin-1-yl]-Propyl]}Carboxamido-5-Methoxycarbonyl-4-Methoxymethyl-2-oxo-1,2,3,6-Tetrahydropyrimidine Dihydrochloride

5-Methoxycarbonyl-4-Methoxymethyl-1,2,3,6-Tetrahydro-2-oxo-6-(3,4-DIFLUOROPHENYL)-PYRIMIDINE:

Copper(I) oxide (5.06 g, 0.035 mole) and acetic acid (2.05 mL) were added sequentially to a stirring solution of methyl 4-methoxyacetoacetate (50.0 g, 0.351 mol), 3,4-difluorobenzaldehyde (51.4 g, 0.351 mmol), and urea (31.6 g, 0.527 mole) in THF (300 mL) at room temperature, followed by dropwise addition of boron trifluoride diethyl etherate (56.0 mL, 0.456 mole). The mixture was stirred at reflux temperature for 8 h, whereupon TLC (1/1 EtOAc/hexanes) indicated completion of the reaction. The reaction mixture was cooled and poured into a mixture of ice and sodium bicarbonate (100 g) and the resulting mixture was filtered through Celite. The Celite pad was washed with dichloromethane (400 mL). The organic layer was separated from the filtrate and the aqueous layer was extracted with more dichloromethane (3×300 mL). The combined organic extracts were dried (sodium sulfate) and the solvent was evaporated. The crude product was purified by flash chromatography (ethyl acetate/hexanes, 1/1;then ethyl acetate), giving the desired product as a pale yellow foam. The foam was triturated with hexanes, giving a white powder (103.3 g, 94%). ¹H NMR δ 3.476 (s, 3H), 3.651 (s, 3H), 4.653 (s, 2H), 5.39 (s, 1H), 6.60 (br s, 1H, NH), 7.00-7.20 (m, 3H), 7.72 (br s, 1H, NH).

(+)-5-Methoxycarbonyl-4-Methoxymethyl-1,2,3,6-Tetrahydro -2-oxo-6-(3,4-Difluorophenyl)-Pyrimidine:

The racemic intermediate 5-methoxycarbonyl-4-methoxymethyl-1,2,3,6-tetrahydro-2-oxo-6-(3,4-difluorophenyl)pyrimidine was resolved by chiral HPLC [Chiralcel OD 20×250 mm #369-703-30604; lambda 254 nm; hexanes/ethanol 90/10; 85 mg per injection; retention time of the desired enantiomer: 16.94 min., the first enantiomer peak to elute], giving (+)-5-methoxycarbonyl-4-methoxymethyl-1,2,3,6-tetrahydro-2-oxo-6-(3,4-difluorophenyl)-pyrimidine (40-42 wt % isolation of the desired enantiomer from the racemate); [α]_{D}=+83.8 (c=0.5, chloroform).

(+)-5-Methoxycarbonyl-4-Methoxymethyl-1,2,3,6-Tetrahydro -2-oxo-6-(3,4-Difluorophenyl)-1-[(4-Nitrophenyloxy)Carbo nyl]Pyrimidine:

A solution of lithium hexamethyldisilazide in THF (1M, 18.0 mL, 18.0 mmol) was added over 2-3 min. to a solution of (+)-5-methoxycarbonyl-4-methoxymethyl-1,2,3,6-tetrahydro-2-oxo-6-(3,4-difluorophenyl)-pyrimidine (1.98 g, 6.34 mmol) in anhydrous THF (20 mL) at −78° C. under argon atmosphere and the mixture was stirred for 10 min. The resulting solution was added over 6 min., via a cannula, to a stirred solution of 4-nitrophenyl chloroformate (4.47 g, 22.2 mmol) in THF (20 mL) at −78° C. The mixture was stirred for an additional 10 min. and the mixture was poured onto ice (50 g) and extracted with chloroform (2×50 mL). The combined extracts were dried (sodium sulfate) and the solvent evaporated. The residue was purified by flash chromatography (hexanes/ethyl acetate, 4/1 to 3.5/1), giving the product as a yellow syrup, which on trituration with hexanes became a white powder (2.40 g, 79%). ¹H NMRδ 3.52 (s, 3H), 3.74 (s, 3H), 4.65-4.80 (q, J=16.5 Hz, 2H), 6.32 (s, 1H), 7.10-7.30 (m, 4H), 7.36 (d, J=9 Hz, 2H), 8.27 (d, J=9 Hz, 2H).

(+)-6-(3,4-Difluorophenyl)-1-(N-[4-(2-Pyridyl)Piperidin-1-yl]-Propyl]}Carboxamido-5-Methoxycarbonyl-4-Methoxymethyl-2-oxo-1,2,3,6-Tetrahydropyrimidine Dihydrochloride:

A solution of (+)-5-methoxycarbonyl-4-methoxymethyl-1,2,3,6-tetrahydro-2-oxo-6-(3,4-difluorophenyl)-1-[(4-nitropheny loxy)carbonyl]pyrimidine (2.38 g, 5 mmol), 3-aminopropyl-4-(2-pyridyl)piperidine (1.21 g, 5.5 mmol) in THF (20 mL) was stirred at room temperature for 12 h.

The solvent was evaporated and the residue was re-dissolved in ethyl acetate (100 mL). The resulting solution was washed with ice-cold 1 N NaOH (4×50 mL), brine (2×50 mL) and dried over potassium carbonate. The solvent was evaporated in vacuo and the residue was purified by flash chromatography (dichloromethane/MeOH/2 M ammonia in MeOH, 980/10/10 to 940/30/30), giving a clean fraction of the desired product (2.45 g, 88%) as a foam and a slightly impure fraction (0.30 g, 10%). ¹H NMR δ 1.60-2.00 (m, 6H), 2.05-2.15 (m, 2H), 2.38-2.43 (br t, 2H), 2.65-2.80 (m, 1H), 3.05-3.06 (br d, 2H), 3.30-3.45 (m, 2H), 3.48 (s, 3H), 3.704 (s, 3H), 4.68 (s, 2H), 6.68 (s, 1H), 7.05-7.20 (m, 5H), 7.58-7.63 (dt, 1H), 7.70 (s, 1H, NH), 8.50-8.52 (dd, 1H), 8.88 (br t, 1H).

The HCl salt was prepared by treatment of a solution of the free base in ether with 1 N HCl in ether. The white powder was dried under reduced pressure: ¹H NMR δ 2.05-2.20 (m, 4H), 2.77-2.88 (m, 2H), 3.00-3.20 (m, 4H), 3.35-3.47 (m, 2H), 3.47 (s, 3H), 3.64-3.70 (m, 2H), 3.71 (s, 3H), 4.05 (br t, 1H), 4.67 (s, 2H), 6.59 (s, 1H) 7.05-7.20 (m, 3H), 7.79 (t, 1H), 8.00 (d, 1H), 8.43 (dt, 1H), 8.96 (br t, 1H, NH), 12.4 (br s, 1H). m.p. 188-191° C.; [α]_{D}=+141.13 (c=0.265, MeOH); Anal. Calcd. for C₂₈H₃₄N₅O₅F₂Cl+0.6 H₂O:C, 52.36; H, 5.84; N, 10.90. Found: C, 52.24; H, 5.96; N, 10.80. (Note: NMR analysis of this product did not show the presence of any water. However, it was noted by the lab that performed the elemental analysis that this sample gains weight during handling by absorbing water from the atmosphere).

### EXAMPLE 18

(1)-1,2,3,6-Tetrahydro-1-{N-[4-(Isobenzofuran)Piperidine -1-yl]-Propyl}Carboxamido-5-Methoxycarbonyl-2-oxo-6-(3,4-Benzofurazan)-4-Methylpyrimidine Hydrochloride

4-(3,4-Benzofurazan)-6-Methyl-2-oxo-3-{[3-(4-Spiro[Isobe nzo-Furan-1(3H),4′-Piperidine]Propyl}-1,2,3,4-Tetrahydropyri Midine-5-Carboxylic Acid Methyl Ester:

1-(3-Aminopropyl)-4-spiro[iso-benzofuran-1 (3H),4′-piperidine] (0.028 g, 0.110 mmol) was added to (±)-6-(benzofurazan)-1,6-dihydro-2-methoxy-5-methoxycarbonyl-4-methyl-1-(4-nitrophenoxy) carbonylpyrimidine (0.047 g, 0.100 mmol) in dry dichloromethane (10 mL) and the solution was stirred at room temperature for 24 h. Aquesous 6 N HCl (2 mL) was added to the reaction mixture which was stirred for another 1 h. The reaction mixture was basified with aqueous 10% KOH solution (pH=9) and extracted into dichloromethane (3×10 mL). The organic layer was dried over sodium sulfate, filtered and concentrated. The crude product was purified by flash chromatography (EtOAc/ MeOH, 4.5/0.5), giving the desired product (41.0 mg, 73%) as a syrup: ¹H NMR δ 1.76-1.81 (m, 7H), 1.94-2.04 (m, 6H), 2.32-2.48 (m, 1H), 2.83 (d, J=10.6 Hz, 2H), 3.36-3.43 (m, 2H), 3.75 (s, 3H), 5.05 (s, 2H), 6.83 (s, 1H), 7.07-7.27 (m, 4H), 7.54 (d, J=9.5 Hz, 1H), 7.69 (s, 1H), 7.78 (d, J=9.5 Hz, 1H), 8.85 (d, J=5.2 Hz, 1H).

HCl in ether (1 N, 5 mL) was added to the free base (0.041 g, 0.073 mmol) in dichloromethane (4 mL), and the solution was concentrated under reduced pressure. The product was recrystallized from ether, giving the hydrochloride salt as a pale yellow solid (42.0 mg, 96%); mp 180-182° C.; Anal. Calcd. for C₂₉H₃₄N₆O₆Cl+0.5 moles H₂O: C, 57.47; H, 5.65; N, 13.87. Found: C, 57.42; H, 5.71; N, 13.70.

### EXAMPLE 19

2-(3,4-Difluorophenyl)4,5-Dihydroimidazole-1-Carboxylic Acid {3-[4-Phenyl-4-(4-Bromo-5-Methylthiopnen-2-yl)]-Propyl)-Amide:

Anal. Calcd. for C₃₀H₃₀N₄O₅ClF₃+HCl+1.5 H₂O: C, 55.26; H, 6.03; N, 8.59. Found: C, 55.29; H, 5.95; N, 8.39.

### EXAMPLE 20

4-(3,4-Difluorphenyl)-6-Methyl-2-oxo-3-{[3-(4-Spiro[Isob enzo-Furan-1(3H),4′-Piperidine]Propyl}-1,2,3,4-Tetrahydropyrimidine-5-Carboxylic Acid Methyl Ester

For the preparation of the ether piperidine precursor of the compound of Example 20,refer to W. E.Parham et al, J. Org. Chem. (1976) 41, 2268.

1-Tert-Butoxycarbonyl-3-(4-Spiro[Isobenzofuran-1(3H),4′-Piperidine])Propylamine:

N-(tert-utoxycarbonyl)-3-bromo-propylamine (0.772 g, 3.27 mmol) and potassium carbonate (0.904 g, 6.54 mmol) were added to a stirring solution of the amine (0.566 g, 3.27 mmol) in dioxane (20 mL) and the reaction mixture was heated at reflux temperature for 24 h. The reaction mixture was cooled to room temperature, concentrated and partitioned between chloroform (40 mL) and water (5 mL). The organic layer was dried over sodium sulfate, filtered and concentrated. The crude product was purified by column chromatography (ethyl acetate/methanol, 4.5/0.5), giving the desired product (0.856 g, 79%) as a colorless oil; ¹H NMR δ 1.45 (s, 9H), 1.63-2.04 (m, 6H), 2.33-2.52 (m, 4H), 2.87 (d, J=11.0 Hz, 2H), 3.2 (br s, 2H), 5.07 (s, 2H), 5.6 (br s, 1H), 7.13-7.28 (m, 4H).

3-(4-Spir[Isobenzo-Furan-1(3H),4′-Piperidine])Propylamine:

Trifluoroacetic acid (1 mL) was added to 1-tert-butoxycarbonyl 3-(4-spiro[isobenzo-furan-1(3H),4′-piperidine])propylamine (0.500 g, 1.51 mmol) in dichloromethane (5 mL) and the solution was stirred at room temperature for 1 h. The reaction mixture was concentrated, neutralized with 10% KOH solution and extracted into dichloromethane (25 mL). The organic layer was dried over sodium sulfate, filtered and concentrated, giving the desired amine (0.340 g, 98%) which was used in the subsequent step without further purification.

4-(3,4-Difluorphenyl)-6-Methyl-2-oxo-3-{[3-(4-Spiro[Isob enzo-Furan-1(3H),4′-Piperidine]Propyl}-1,2,3,4-Tetrahydropyrimidine-5-Carboxylic Acid Methyl Ester:

3-(4-spiro[isobenzo-furan-1(3H),4′-piperidine])propylamine (0.0319 g, 0.123 mmol) was added to (±)-6-(3,4-Difluorophenyl)-1,6-dihydro-2-methoxy-5-methoxycarbonyl-4-methyl-1-(4-nitrophenoxy)c arbonylpyrimidine (0.052 g, 0.112 mmol) in dry dichloromethane (10 mL) and the solution was stirred at room temperature for 24 h. Aqueous 6 N HCl (2 mL) was added and the reaction mixture was stirred for an additional 1 h. After neutralization with 10% aqueous KOH solution, the reaction mixture was extracted with dichloromethane (3×10 mL). The organic layer was dried over sodium sulfate, filtered and concentrated. The crude product was purified by flash chromatography (EtOAc/ MeOH, 4.5/0.5), giving the desired product (0.040 g, 64%) as a syrup; 1H-NMR δ 1.73-1.78 (m, 7H), 1.93-2.04 (m, 2H), 2.33-2.48 (m, 6H) 2.83 (d, J=11.8 Hz, 2H), 3.35-3.41 (m, 2H), 3.71 (s, 3H), 5.06 (s, 2H), 6.75 (s, 1H) 7.04-7.26 (m, 7H), 8.82 (t, J=5.1 Hz, 1H).

A solution of 1 N HCl in ether (5 mL) was added to the free base (0.040 g, 0.072 mmol) in dichloromethane (4 mL) and the solution was concentrated in vacuo. The product was recrystallized from ether, giving the dihydrochloride as a pale yellow solid (0.042 g, 99%); mp 178-182° C.; Anal. Calcd. for C₂₉H₃₄F₂N₄O₅Cl₂+0.6 H₂O: C, 57.87; H, 5.73, N 9.31. Found: C, 58.11; H 5.90; N 8.95.

### EXAMPLE 21

1,2,3,6-Tetrahydro-1-{N-[4-(Dihydroindene)-1-yl}Propyl}C arboxamido-5-Methoxycarbonyl-2-oxo-6-(3,4-Benzofurazan)-4-Methylpyrimid-ine

For the preparation of the indane piperidine precursor of the compound of Example 21, refer to M. S.Chambers J. Med. Chem. (1992) 35,2033.

N-(tert-butoxycarbonyl)3-(4-spiro[isobenzo-furan-1(3H),4′-piperidine])propylamine(1.10 g, 4.64 mmol) and potassium carbonate (1.17 g, 8.44 mmol) were added to a stirring solution of the amine (0.790 g, 4.22 mmol) in dioxane (20 ml), and the resulting solution was heated at reflux temperature for 24 h. The reaction mixture was cooled to room temperature, concentrated and partitioned between chloroform (40 mL) and water (5 mL). The organic layer was dried over sodium sulfate, filtered and concentrated. The crude product was purified by column chromatography (ethyl acetate/ methanol, 4.5/0.5), giving the desired product (0.886 g, 61%) as a colorless oil; ¹H NMR δ 1.46 (s, 9H), 1.55 (d, J=11.3 Hz, 2H), 1.69 (t, J=6.3 Hz, 2H), 1.88-2.47 (m, 6H), 2.47 (t, J=6.3 Hz, 2H), 2.88 (t, J=3.3 Hz, 4H), 3.23 (d, J=5.6 Hz, 2H), 5.85 (br s, 1H), 7.18 (s, 4H).

Trifluoroacetic acid (1 ml) was added to 1-tert-butoxycarbonyl-3-(4-spiro [isobenzo-furan-1 (3H),4′ -piperidine] )propylamine(0.180 g, 0.52 mmol) in dichloromethane (5 ml) and the resulting solution was stirred at room temperature for 1 hour. The solution was concentrated, neutralized with 10% KOH solution and extracted into dichloromethane (25 ml). The organic layer was dried over sodium sulfate, filtered and concentrated, giving propylamine (0.156 g, 100%) which was used in the subsequent step without further purification.

(±)-4-(3,4-Benzofurazan)-6-Methyl-2-oxo-3-{Spiro[1H-inda ne-1,4′-Piperidine]Propyl}-1,2,3,4-Tetrahydropyrimidine-5-Carboxylic Acid Methyl Ester Hydrochloride:

To (±)-4-(3,4-benzofurazan)-1,6-dihydro-2-methoxy-5-methoxycarbonyl-4-methyl-1-(4-nitrophenoxy)-carbonylpyrimidine (0. 059 g, 0.126 mmol) in dry dichloromethane (10 m L) 1-(3-aminopropyl)spiro[1H-indane-1,4′- piperidine] (0.062 g, 0.252 mmol) was added and the solution was stirred at room temperature for 24 h. The reaction mixture was stirred for another 1 h after addition of 2 mL of 6N HCl. The reaction mixture was basified with 10% aqueous KOH solution (pH=9) and extracted with dichloromethane (3×10 mL). The combined organic extracts were dried over sodium sulfate, filtered and concentrated. The crude product was purified by flash chromatography (EtOAc/ MeOH, 4.5/0.5), giving 0.070 g (100%) of the desired product as a syrup: ¹H NMRδ 1.51 (d, J=12.5 Hz, 2H), 1.76-2.08 (m, 4H), 2.12 (t, J=10.3 Hz, 2H), 2.45 (s, 5H), 2.86-2.91 (m, 4H), 3.30-3.45 (m, 2H), 3.75 (s, 3H), 6.83 (s, 1H), 7.02 (br s, 1H), 7.0 (m, 4H), 7.54 (d, J=9.6 Hz, 1H), 7.69 (s, 1H), 7.78 (d, J=9.2 Hz, 1H), 8.84, (t, J=5.2 Hz, 1H).

To the free base (0.070 g, 0.125 mmol) in 4 mL of dichloromethane, 5 mL of 1 N HCl in ether was added, and the solution was concentrated under reduced pressure. Recrystallization from ether gave 0.088 g (100%) of (±)-4-(3,4-benzofurazan)-6-methyl-2-oxo-3-{spiro[1H-inda ne-1,4′-piperidine]propyl}-1,2,3,4-tetrahydropyrimidine-5-c arboxylic acid methyl ester hydrochloride as a white solid: m.p. 155-157° C.; Anal. Calcd. for C₃₀H₃₆N₆O₅Cl: C, 57.12; H, 5.76; N, 13.33. Found: C, 57.40; H, 5.96; N, 13.02.

### EXAMPLE 22

(+)-1,2,3,6-Tetrahydro-1-{N-[4-(Benzo-4′,5′(H)Furan)Pipe ridin-1-yl] Propyl}Carboxamido-4-Ethyl-6-(3,4-difluorophenyl)-2-oxo-Pyrimidine-5-Carboxamide Hydrochloride:

DMAP ECD (0.250 mmol, 0.050 g) was added to a stirred mixture of (+)-1,2,3,6-tetra-hydro-1-{N-[4-(benzo-4′,5′(h)furan)-piperidin-1-yl]propyl}carbox-amido-4-ethyl-6-(3,4-difluorophenyl)-2-oxo-pyrimidine-5-carboxyl-ic acid hydrochloride (0.100 mmol, 0.055 g) and N-methylmorpholine (0.330 mL) in dry dichloromethane (10 mL). The resulting mixture was stirred at room temperature for 1 h and quenched with NH₃. The reaction mixture was stirred at room temperature overnight, concentrated and chromatographed, giving the desired product. The HCl salt was prepared by the addition of HCl in ether to a solution of the product in dichloromethane, followed by evaporation of the solvents. Anal. Calc. For C₂₉H₃₃N₅O₄ F₂+HCl+0.7 CHCl₃: C, 52.96; H, 5.29; N, 9.40. Found: C, 52.81; H, 5.69; N, 8.97.

### EXAMPLE 23

(1)-1,2,3,6-Tetrahydro-1-{N-[4-(3,4-Dihydro-2-Oxospiro-Naphthalene-1(2H))-Piperidine-1-yl]Propyl}Carboxamido-5-Methoxycarbonyl-2-oxo-6-(3,4-Benzofurazan)-4-Methylpyrimidine Hydrochloride

1-(3-Tert-Butoxycarbonylaminopropyl)Spiro[Isochroman-3,4′Piperidin]-1-One:

To a stirred solution of spiro[piperidine-4,1′-tetralin] To a stirred solution of spiro[isochroman-3,4′-piperidin]-1-one (K.Hashigaki et al. Chem.Pharm.Bull. (1984) 32, 3568.) (0.587 g, 2.58 mmol) in dioxane (20 mL), N-(tert-butoxycarbonyl)-3-bromopropylamine (0.615 g, 2.84 mmol) and potassium carbonate (0.714 g, 5.17 mmol) were added and the solution was refluxed for 24 h. The reaction mixture was cooled to room temperature, concentrated and partitioned between 40 mL chloroform and 5 mL water. The organic layer was dried over sodium sulfate, filtered and concentrated. The crude product was purified by column chromatography (ethyl acetate/ methanol, 4.5/0.5) to yield 0.465 g (47%) of the desired product as a colorless oil; ¹H NMR δ 1.45 (s, 9H), 1.64-2.18 (m, 7H), 2.45-2.84 (m, 6H), 3.19-3.95 (m, 4H), 6.01 (br s, 1H), 7.13-7.26 (m, 3H), 7.42 (d, J=7.7H)

Step B. 1-(3-Aminopropyl)Spiro [Isochroman-3,4′Piperidin]-1-One:

To 1-(3-tert-Butoxycarbonylaminopropyl)-spiro(isochroman-3,4′-piperidin]-1-one (0.144 g, 0.375 mmol) in 5 mL of dichloromethane, 1 mL of trifluoroacetic acid was added and the solution stirred at room temperature for 1 h. The solution was concentrated, neutralized with 10% KOH solution and extracted into 25 mL of dichloromethane. The organic layer was dried over sodium sulfate, filtered and concentrated, giving 0.110 g (100%) of the product which was used as such for the subsequent step.

(±)-4-(3,4-Benzofurazan)-6-Methyl-2-oxo-3-{(Spiro[Isochr oman-3,4′-Piperidin]-1-One)Propyl}-1,2,3,4-Tetrahydropyrimidine-5-Carboxyl-ic Acid Methyl Ester:

To (±)-4-(3,4-Benzofurazan)-1,6-dihydro-2-methoxy-5-methoxy carbonyl-4-methyl-1-(4-nitrophenoxy)-carbonylpyrimidine (40.0 mg, 0.0865 mmol) in 10 mL of dry dichloromethane, spiro[isochroman-3,4′piperidin]-1-one (44.0 mg, 0.173 mmol) was added and the solution was stirred at room temperature for 24 h. The reaction mixture was stirred for another 1 h after addition of 2 mL of 6N HCl. The reaction mixture was basified with 10% aqueous KOH solution (pH=9) and extracted into dichloromethane (3×10 mL). The organic layer was dried over sodium sulfate, filtered and concentrated. The crude product was purified by flash chromatography (EtOAc/ MeOH, 4.5/0.5), giving 50.0 mg (100%) of the desired product as a syrup: ¹H NMR δ 1.67-2.13 (m, 8H), 2.45 (m, 5H), 2.70 (t, J=7.4 Hz, 2H), 2.72-2.75 (m, 2H), 3.19 (t, J=7.4 Hz, 2H), 3.34-3.45 (m, 2H), 3.75 (s, 3H), 6.82 (s, 1H), 6.87 (s, 1H), 7.13-7.44 (m, 3H), 7.54 (d, J=9.6 Hz, 1H), 7.43 (d, J=7.4 Hz, 1H), 7.69 (s, 1H), 7.79 (d, J=9.6 Hz, 1H), 8.87 (t, J=5.2 Hz, 1H).

To the free base (50.0 mg, 0.084 mmol) in 4 mL of dichloromethane, 5 mL of 1 N HCl in ether was added, and the solution concentrated under reduced pressure. Recrystallization from ether gave 30.0 mg (86%) of the product as a white solid: m.p. 165-167° C.; Anal. Calcd. for C₃₁H₃₆N₆O₆Cl+1.5 H₂O: C, 57.81; H, 5.95. Found: C, 57.75; H, 5.91.

### EXAMPLE 24

(1)-1,2,3,6-Tetrahydro-1-{N-[4-(3,4-Dihydro-2-Oxospiro-Naphthalene-1(2H))-Piperidine-1-yl]Propyl}Carboxamido-5-Methoxy-Carbonyl-2-oxo-6-(3,4-Difluorophenyl)-4-Methylpyrimidine

(±)-4-(3,4-Difluorophenyl)-6-Methyl-2-oxo-3-{ (Spiro[Isoc hroman-3,4′Piperidin]-1-One)Propyl}-1,2,3,4-Tetrahydropyrimidine-5-Carboxylic Acid Methyl Ester:

To (±)-4-(3,4-Difluorophenyl)-1,6-dihydro-2-methoxy-5-methoxycarbonyl-4-methyl-1-(4-nitrophen-oxy)carbonylpyrimidine (40.0 mg, 0.0865 mmol) in 10 mL of dry dichloromethane, spiro [isochroman-3,4′piperidin]-1-one (44.0 mg, 0.173 mmol) was added and the solution was stirred at room temperature for 24 h. The reaction mixture was stirred for another 1 h after addition of 2 mL of 6N HCl. The reaction mixture was basified with 10% aqueous KOH solution (pH 9) and extracted into dichloromethane (3×10 mL). The organic layer was dried over sodium sulfate, filtered and concentrated. The crude product was purified by flash chromatography (EtOAc/ MeOH, 4.5/0.5), giving 45.0 mg (90%) of (+)-4-(3,4-difluorophenyl)-6-methyl-2-oxo-3-{(spiro[isochroman-3,4′piperidin]-1-one)propyl}-1,2,3,4-tetrahydropyrimi-dine-5-carboxylic acid methyl ester as a syrup; ¹H NMR δ 1.75-1.94 (m, 9H), 2.05-2.13 (m, 4H), 2.36-2.41 (m, 5H), 2.70 (t, J=7.35 Hz, 2H), 2.77 (m, 2H), 3.19 (t, J=7.4 Hz, 2H), 3.39-3.43 (m, 2H), 6.69 (s, 1H), 7.04-7.45 (m, 8H), 8.82 (t, J=5.2 Hz, 1H).

To the free base (45.0 g, 0.077 mmol) in 4 mL of dichloromethane, 5 mL of 1 N HCl in ether was added, and the solution was concentrated in vacuo. Recrystallization from ether gave 0.050 g (100%) of (±)-4-(3,4-difluorophenyl)-6-methyl-2-oxo-3-{ (spiro-[isochroman-3,4′piperidin]-1-one)propyl}-1,2,3,4-tetrahydro-pyrimidine-5-carboxylic acid methyl ester hydrochloride as a white solid: m.p. 150-152° C.; Anal. Calcd. for C₃₁H₃₈F₂N₄OCl+2H₂O: C, 56.49; H,5.96. Found: C, 56.40; H, 5.95.

### EXAMPLE 25

5-[(Z)-1-(1-Ethyl-2,2,4-Trimethyl-1,2-Dihydro-6-quinolin yl)-Methylidene]-2-Thioxo-1,3-Thiazolan-4-One

### EXAMPLE 26

1-[Bis(4-Fluorophenyl)Methyl]-4-(3-Phenyl-2-Propenyl)Pip erazine

### EXAMPLE 27

4-[(4-Imidazo[1,2-A]Pyridin-2-Ylphenyl)Imino]Methyl-5-Me thyl-1,3-Benzenediol

### EXAMPLE 28

1-[3-(4-Chlorobenzoyl)]Propyl-4-Benzamidopiperidine Preparation of 1-[3-(4-chlorobenzoyl)propyl]-4-benzamidopiperidine 1-[3-(4-Chlorobenzoyl)Propyl]-4-Benzamidopiperidine:

A mixture of 3-(4-chlorobenzol)propyl bromide (640 mg, 2.45 mmol), 4-benzamidopiperidine (500 mg, 2.45 mmol) and K₂CO₃ (1.01 g, 7.34 mmol) in 50 ml of acetone was heated at reflux temperature for 48 h. The cooled reaction mixture was filtered to remove the solids, concentrated in vacuo, giving a yellow solid, which was purified by chromatography (MeOH/CHCl₃, 5/95). The product (320 mg , 33.9%) was isolated as a white powder: ¹H NMR δ 1.46 (dq, J1=1.0 Hz, J2=8.4 Hz, 2H), 1.90-2.10 (m, 4H), 2.16 (m, 2H), 2.43 (t, J=6.9 Hz, 2H), 2.80-2.90 (m, 2H), 2.97 (t, J=6.9 Hz, 2H), 3.97 (m, 1H), 5.92 (d, J=7.8 Hz, 1H, N-H), 7.40-8.00 (m, 9H). The product was converted to the HCl salt and recrystallized from MeOH/Et₂O, m.p. 243-244° C.; Anal. Calcd for C₂₂H₂₅ClN₂O₂+HCl+H₂O: C, 60.15; H, 6.37; N, 6.37; Found: C, 60.18; H, 6.34; N, 6.29.

### EXAMPLE 29

4-[4-(4-Chlorophenyl)-4-Hydroxy-1-Piperidinyl]-1-(4-Chlo rophen-yl)-1-Butanone

### EXAMPLE 30

N-Methyl-8-[4-(4-Fluorophenyl)-4-Oxobutyl]-1-PHENYL-1,3, 8-Tri-azaspiro-[4.5]Decan-4-one

### EXAMPLE 31

1H-1,2,3-Benzotriazol-1-yl (2-Nitrophenyl) Sulfone

### EXAMPLE 32

(1)-1,2,3,6-Tetrahydro-1-{N-[4-(Dihydroindene)-1-yl}Prop yl}-Carboxamido-5-Methoxycarbonyl-2-oxo-6-(3,4-Difluoro)-4-M ethyl-Pyrimidine

1-(3-Tert-Butoxycarbonylaminopropyl)Spiro[1H-Indane-1,4′-Piperidine]:

To a stirred solution of spiro[1H-indane-1,4¹-piperidine] (M. S.Chambers et al. J. Med. Chem. (1992) 35, 2033.) (0.790 g, 4.22 mmol) in dioxane (20 mL), N-(tert-butoxy-carbonyl)-3-bromopropylamine (1.1 g, 4.64 mmol) and potassium carbonate (1.17 g, 8.44 mmol) were added and the resulting solution was heated at reflux temperature for 24 h. The reaction mixture was cooled to room temperature, concentrated and partitioned between 40 mL of chloroform and 5 mL of water. The organic layer was dried over sodium sulfate, filtered and concentrated. The crude product was purified by column chromatography (ethyl acetate/ methanol, 4.5/0.5) to yield 0.886 g (61%) of the required product as a colorless oil: ¹H NMR δ 1.46 (s, 9H), 1.55 (d, J=11.3 Hz, 2H), 1.69 (t, J=6.3 Hz, 2H), 1.88-2.47 (m, 6H), 2.47 (t, J=6.3 Hz, 2H), 2.88 (t, J=3.3 Hz, 4H), 3.23 (d, J=5.6 Hz, 2H), 5.85 (br s, 1H), 7.18 (s, 4H).

1-(3-Aminopropyl)Spiro[1H-Indane-1,4′-Piperidine]:

To 1-(3-tert- Butoxycarbonylaminopropyl) spiro [1H-indane-1,4-piperidine] (0.180 g, 0.52 mmol) in 5 mL of dichloromethane, 1 mL of trifluoroacetic acid was added and the solution stirred at room temperature for 1 h. The solution was concentrated, neutralized with 10% KOH solution and extracted into 25 mL of dichloromethane. The organic layer was dried over sodium sulfate, filtered and concentrated, giving 0.156 g (100%) of the product which was used as such for the subsequent step.

(±)-4-(3,4-Difluoro)-6-Methyl-2-oxo-3-{Spiro[1H-Indane-1 ,4′-Piperidine]Propyl}-1,2,3,4-Tetrahydropyrimidine-5-Ca rboxylic Acid Methyl Ester:

To (±)-4-(3,4-difluoro)1,6-dihydro-2-methoxy-5-methoxycarbonyl-4-methyl-1-(4-nitrophenoxy)carbonylpyrimidine (50.0 g, 0.108 mmol) in 10 mL of dry dichloromethane, 1-(3-aminopropyl)spiro[1H-indane-1,4′-piperidine] (53.0 mg, 0.216 mmol) was added and the solution was stirred at room temperature for 24 h. The reaction mixture was stirred for another 1 h after addition of 2 mL of 6N HCl. The reaction mixture was basified with 10% aqueous KOH solution (pH=9) and extracted into dichloromethane (3×10 mL). The organic layer was dried over sodium sulfate, filtered and concentrated. The crude product was purified by flash chromatography (EtOAc/ MeOH, 4.5/0.5), giving 60.0 mg (100%) of the product as a syrup: ¹H NMR δ 1.52 (d, J=13.2 Hz, 2H), 1.70-2.07 (m, 8H), 2.12 (t, J=10.3 Hz, 2H), 2.42 (s, 4H), 2.86-2.91 (m, 3H), 3.32-3.43 (m, 2H), 3.72 (s, 3H), 6.71 (s, 1H), 6.81 (br s, 1H), 7.04-7.19 (m, 7H), 8.82 (t, J=5.2 Hz, 1H).

To the free base (0.060 g, 0.108 mmol) in 4 mL of dichloromethane, 5 mL of 1 N HCl in ether was added, and the solution was concentrated under reduced pressure. Recrystallization from ether gave 0.070 g (100%) of the product as a white solid; m.p. 150-153° C.; Anal. Calcd. for C₃₀H₃₆F₂N₄O₆Cl: C, 54.86; H,5.53; N, 8.54. Found: C, 54.96; H, 5.57; N, 8.27.

### EXAMPLE 33

(+)-1,2,3,6-Tetrahydro-1-{N-[4-(3,4,5-Trifluoro)-Phenyl-Piper-idin-1-yl]Propyl}Carboxamido-4-Methoxymethyl -6-(3,4-Difluorophenyl)-2-Oxopyrimidine-5-Carboxylic Acid Methyl Ester:

mp ° C.; [α]_{D}+123.0, (c=0.15, MeOH); ¹H NMR δ 1.70-1.82 (m, 6H), 1.97-2.08 (m, 2H), 2.40 (t, J=6.9 Hz, 2H), 2.74-2.87 (m, 1H), 3.01 (d, J=11.1 Hz, 2H), 3.29-3.40 (m, 2H), 3.49 (s, 3H), 3.71 (s, 3H), 4.69 (s, 2H), 6.68 (s, 1H), 6.88-6.95 (m, 2H), 7.05-7.11 (m, 2H), 7.15-7.22 (m, 1H), 7.71 (s, 1H), 8.90 (t, J=5.4 Hz, 1H)

### EXAMPLE 34

(+)-1,2,3,6-Tetrahydro-1-{N-[2-(S)-Methyl)-4-(2-Nitrophe ny)-Piperazin-1yl]Propyl}-Carboxamido-4-Methyl-6-(3,4-Difluo rophen-yl)-2-oxo-pyrimidine

(S)-(+)-3-Methyl-1-(2-Nitrophenyl)-Piperazine:

To a solution of 2-bromonitrobenzene (0.600 g, 3.00 mmol) in 1,4-dioxane (15 mL) was added (S)-(+)-2-methylpiperazine (0.500 g, 0.500 mmol) and powdered K₂CO₃ (15.0 mmol, 1.50 g) and the resulting suspension was heated at reflux for 10 h. After the suspension was cooled, it was filtered through a sintered glass funnel and the solvent was removed in vacuo. The resulting residue was purified by column chromatography (1/1 hexane/EtOAc followed by 4/1 EtOAc/MeOH ), giving (S)-(+)-3-methyl-1-(2-nitrophenyl)-piperazine as an orange oil (0.53 g, 80%).

(+)-1,2,3,6-Tetrahydro-1-{N-[2-(S)-Methyl)-4-(2-Nitrophe nyl) Piperazin-1yl] Propyl}-Carboxamido-4-Methyl-6-(3,4-Di fluorophenyl)-2-oxo-Pyrimidine:

To a solution of (+)-1-(3-bromo-propylcarbamoyl)-6-(3,4-difluorophenyl)-4-methyl-2-oxo-1,6-dihydro-pyrimi dine-5-carboxylic acid methyl ester (0.200 g, 0.500 mmol) and (S)-(+)-3-methyl-1-(2-nitrophenyl)-piperazine (0.170 g, 0.750 mmol) in 20 mL of anhydrous acetone was added powdered K2CO₃ (0.34 g, 3.5 mmol) and KI (0.07 g, 0.5 mmol) and the resulting suspension was heated at reflux temperature for 10 h. TLC indicated a new spot for the product (Rf=0.3, 3/0.5 EtOAc/MeOH) and mostly the starting material. The suspension was cooled, filtered and the solvent was evaporated and the residue was purified by column chromatography (EtOAc/MeOH, 5/1). (+)-1,2,3,6-Tetrahydro-1-{N-[2-(S)-methyl)-4-(2-nitrophenyl)piperazi n-1-yl]-propyl}-carboxamido-4-methyl-6-(3,4-difluorophenyl)-2-oxo-pyr-imidine was obtained as yellow oil (0.030 g, 10% yield). The HCl salt was prepared by the addition of HCl in ether to a solution of the product in dichloromethane, followed by evaporation of the solvents; mp 150-153° C.; [α]_{D}=58.3 (c=0.3, MeOH); ¹H NMR (CD₃OD)d 1.04 (d, J=6.0 Hz, 3H), 1.71-1.78 (m, 2H), 2.33-2.49 (m, 3H), 2.42 (s, 3H), 2.55-2.92 (m, 5H), 3.00-3.10 (m, 3H), 3.34-3.42 (m, 2H), 3.72 (s, 3H), 6.71 (s, 1H), 7.01-7.32 (m, 6H), 7.46 (dt, J=0.7 Hz, J=8.4 Hz, 1H), 7.74 (dd, J=1.5, 8.4 Hz, 1H), 8.82 (t, J=3.9 Hz, 1H). Anal calcd. for C₂₈H₃₃N₆F₂O₆+0.20 CH₂Cl₂: C, 52.92; H, 5.26; N, 13.13. Found: C, 52.84; H, 5.68; N, 12.94.

### EXAMPLE 35

1,2,3,6-Tetrahydro-1{N-[4-(2′ -Methyl-Phenyl) Piperazin-1-yl]-Propyl}-Carboxamido-4-Methyl-6-(3,4-Difluorophenyl)-2-oxo-Pyrimidine:

The amine used was 4-(2′-methyl-phenyl)-piperazine. ¹H NMR δ 1.75-1. 80 (m, 2H), 2.29 (s, 3H), 2.42 (s, 3H), 2.41-2.48 (m, 2H), 2.58-2.62 (m, 4H), 2.91-2.97 (m, 4H), 3.35-3.42 (m, 2H), 3.72 (s, 3H), 6.71 (s, 1H), 6.97-7.26 (m, 8H), 8.81 (t, J=3.9 Hz, 1H). The product was dissolved in ether and 1 N HCl in ether was added. The ether was evaporated, giving the dihydrochloride salt; mp 66-71° C. Anal calcd. for C₂₈H₃₅N₅F₂O₄ Cl₂+1.75 acetone: C, 55.73; H, 6.40; N, 9.78. Found: C, 56.16; H, 6.29; N, 10.06.

### EXAMPLE 36

(+)-1,2,3, 6-Tetrahydro-5-Methoxycarbonyl-4-Methoxymethyl -2-oxo -1-{N-[3-(4-Methyl-4-Phenyl Piperidine -1-yl]Propyl}-6-(3,4-Difluorophenyl) Pyrimidine:

Hygroscopic; [α]_{D}=+82.1(c=0.31, MeOH); ¹H NMR δ 1.14 (s, 3H), 1.61-1.72 (m, 4H), 2.03-2.08 (m, 2H), 2.25 (t, J=7.2 Hz, 2H), 2.30-2.42 (m, 4H), 3.19-3.31 (m, 2H), 3.40 (s, 3H), 3.63 (s, 3H), 4.60 (s, 2H), 6.60 (s, 1H), 6.97-7.29 (m, 8H), 7.63 (br s, 1H), 8.78 (t, J=5.7 Hz, 1H). Anal calcd. for C₃₀H₃₇N₄O₅F₂Cl+CH₂Cl₂: C, 53.80; H, 5.68; N, 8.10. Found: C, 53.79; H, 6.03; N, 7.83.

### EXAMPLE 37

5-(5-Butyl-2-Thienyl)Pyrido[2,3-d]Pyrimidine-2,4,7 (1H, 3H, 8H).

### EXAMPLE 38

Methyl (4S)-3-[({3-[4-(3-Aminophenyl)-1-Piperidinyl]Propyl}Amino)Carbonyl]-4-(3,4-Difluorophenyl)-6-(Methoxymethyl)-2-oxo-1,2,3,4-Tetrahydro-5-Pyrimidinecarboxylate:

¹H NMR (400 MHz, CDCl₃) δ 7.80 (s, 1H), 7.22-7.02 (m, 2H), 6.95 (t, 2H, J=8.7 Hz), 6.63-6.44 (m, 4H), 4.56 (ABq, 2H), 3.62 (s, 3H), 3.33 (s, 3H), 3.32 (m, 4H), 2.96 (br s, 2H), 2.34 (t, 2H, J=7.5 Hz), 2.11-1.94 (m, 3H), 1.81-1.64 (m, 4H); ESMS m/e: 572.3 (M+H)⁺.

### EXAMPLE 39

The product was obtained according to the method described for Example 40.

Methyl (4S)-4-(3,4-Difluorophenyl)-3-({[3-(4-{3-[(Methoxyacetyl)Amino]Phenyl}-1-Piperidinyl)Propyl]Amino}Carbonyl)-6-(Methoxymethyl)-2-oxo-1,2,3,4-Tetrahydro-5-Pyrimidinecarboxylate:

15.6 mg (69% yield); ¹H NMR (400 MHz, CDCl₃) δ 9.01 (s, 1H), 8.25 (s, 1H), 7.60 (s, 1H), 7.37 (d, 1H, J=7.2 Hz), 7.30-7.05 (m, 5H), 7.02 (d, 1H, J=8.0 Hz), 6.71 (s, 1H), 4.70 (s, 2H), 4.03 (s, 2H), 3.73 (s, 3H), 3.53 (s, 3H), 3.47 (s, 3H), 3.42-3.33 (m, 2H), 3.08 (br s, 2H), 2.49 (br s, 2H), 2.20 (s, 2H), 2.07 (br s, 1H), 1.97-1.75 (m, 4H); ESMS m/e: 644.3 (M+H)⁺.

### EXAMPLE 40

Methyl (4S)-4-(3,4-Difluorophenyl)-3-({[3-(4-{3-[(3,3-Dimethylbutanoyl)Amino]Phenyl}-1-Piperidinyl)Propyl]Amino}Carbonyl)-6-(Methoxymethyl)-2-oxo-1,2,3,4-Tetrahydro-5-Pyrimidinecarboxylate

To the 20 ml vial was added methyl (4S)-3-[({3-[4-(3-aminophenyl)-1-piperidinyl]propyl}amino)carbonyl]-4-(3,4-difluorophenyl)-6-(methoxymethyl)-2-oxo-1,2,3,4-tetrahydro-5-pyrimidinecarboxylate (0.035 mmol), an acid chloride or sulfonyl chloride (1.5 eq), N,N-diisopropylethylamine (5 eq) and dichloromethane (2 ml) at room temperature. The reaction mixture was stirred at room temperature for 24 h, at which time the TLC analysis indicated the reaction was completed. The reaction mixture was concentrated to a small volume and purified by preparative TLC (silica, 2000 microns, 95:5=dichloromethane:methanol with 1% of isopropylamine) to give 5.6 mg of methyl (4S)-4-(3,4-difluorophenyl)-3-({[3-(4-{3-[(3,3-dimethylbutanoyl)amino]phenyl}-1-piperidinyl)propyl]amino}carbonyl)-6-(methoxymethyl)-2-oxo-1,2,3,4-tetrahydro-5-pyrimidinecarboxylate: 24.6% yield; ¹H NMR (400 MHz, CDCl₃) δ 7.50 (s, 1H), 7.26 (d, 1H, J=8.3 Hz), 7.15-7.02 (m, 5H), 6.88 (d, 1H, J=8.3 Hz), 6.55 (s, 1H), 4.56 (ABq, 2H), 3.62 (s, 3H), 3.32 (s, 3H), 3.25 (t, 4H, J=9.0 Hz), 2.99 (d, 2H, J=10.8 Hz), 2.49-2.37 (m, 3H), 2.08 (t, 2H, J=11.7 Hz), 1.78-1.65 (m, 14H); ESMS m/e: 670.4 (M+H)⁺.

### EXAMPLE 41

The product was obtained according to the method described for methyl (4S)-4-(3,4-difluorophenyl)-3-({[3-(4-{3-[(3,3-dimethylbutanoyl)amino]phenyl}-1-piperidinyl)propyl]amino}carbonyl)-6-(methoxymethyl)-2-oxo-1,2,3,4-tetrahydro-5-pyrimidinecarboxylate.

Methyl (4S)-4-(3,4-Difluorophenyl)-6-(Methoxymethyl)-2-oxo-3-{[(3-{4-[3-(Propionylamino)Phenyl]-1-Piperidinyl}Propyl)Amino]Carbonyl)-1,2,3,4-Tetrahydro-5-Pyrimidinecarboxylate:

9.9 mg (45% yield) δ ¹H NMR (400 MHz, CDCl₃) δ 7.36 (s, 1H), 7.28 (d, 1H, J=8.0 Hz), 7.16-7.02 (m, 5H), 6.86 (d, 1H, J=7.6 Hz), 6.54 (s, 1H), 4.56 (ABq, 2H), 3.62 (s, 3H), 3.32 (s, 3H), 3.27-3.19 (m, 4H), 2.95 (d, 2H, J=10.3 Hz), 2.41 (m, 1H), 2.34 (t, 2H, J=7.7 Hz), 2.28 (q, 2H, J=7.6 Hz), 2.01 (t, 2H, J=11.1 Hz), 1.73-1.64 (m, 8H); ESMS m/e: 628.4 (M+H)⁺

### EXAMPLE 42

The product was obtained according to the method described for methyl (4S)-4-(3,4-difluorophenyl)-3-({[3-(4-{3-[(3,3-dimethylbutanoyl)amino]phenyl}-1-piperidinyl)propyl]amino}carbonyl)-6-(methoxymethyl)-2-oxo-1,2,3,4-tetrahydro-5-pyrimidinecarboxylate.

Methyl (4S)-4-(3,4-difluorophenyl)-6-(Methoxymethyl)-3-({[3-(4-{3-[(3-Methylbutanoyl)Amino]Phenyl}-1-Piperidinyl)Propyl]Amino}Carbonyl)-2-oxo-1,2,3,4-Tetrahydro-5-Pyrimidinecarboxylate:

10.4 mg (45% yield) δ ¹H NMR (400 MHz, CDCl₃) δ 7.36 (s, 1H), 7.28 (d, 1H, J=7.9 Hz), 7.16-7.03 (m, 5H), 6.88 (d, 1H, J=7.4 Hz), 6.56 (s, 1H), 4.56 (ABq, 2H), 3.62 (s, 3H), 3.32 (s, 3H), 3.25 (t, 4H, J=6.7 Hz), 2.98 (d, 2H, J=11.1 Hz), 2.43 (m, 1H), 2.38 (t, 2H, J=7.5 Hz), 1.13 (d, 2H, J=7.5 Hz), 2.10-2.01 (m, 2H), 1.75-1.64 (m, 6H), 0.91 (d, 6H, J=5.8 Hz); ESMS m/e: 656.4 (M+H)⁺

### EXAMPLE 43

The product was obtained according to the method 30 described for methyl (4S)-4-(3,4-difluorophenyl)-3-({[3-(4-{3-[(3,3-dimethylbutanoyl)amino]phenyl}-1-piperidinyl)propyl]amino}carbonyl)-6-(methoxymethyl)-2-oxo-1,2,3,4-tetrahydro-5-pyrimidinecarboxylate.

Methyl (4S)-4-(3,4-Difluorophenyl)-3-([(3-{4-[3-(Isobutyrylamino)Phenyl]-1-Piperidinyl}Propyl)Amino]Carbonyl}-6-(Methoxymethyl)-2-oxo-1,2,3,4-Tetrahydro-5-Pyrimidinecarboxylate:

16.4 mg (73% yield) δ ¹H NMR (400 MHz, CDCl₃) δ 7.37 (s, 1H), 7.28 (d, 1H, J=7.3 Hz), 7.16-7.01 (m, 5H), 6.88 (d, 2H, J=7.3 Hz), 6.54 (s, 1H), 4.56 (ABq, 2H), 3.62 (s, 3H), 3.32 (s, 3H), 3.25 (t, 2H, J=6.8 Hz), 3.23-3.18 (m, 2H), 3.03 (d, 2H, J=11.7 Hz), 2.57-2.48 (m, 1H), 2.43 (t, 2H, J=8.0 Hz), 2.14 (t, 2H, J=9.4 Hz), 1.8-1.65 (m, 5H), 1.09 (d, 6H, J=6.3 Hz); ESMS m/e: 642.4 (M+H)⁺

### EXAMPLE 44

The product was obtained according to the method described for methyl (4S)-4-(3,4-difluorophenyl)-3-({[3-(4-{3-[(3,3-dimethylbutanoyl)amino]phenyl}-1-piperidinyl)propyl]amino}carbonyl)-6-(methoxymethyl)-2-oxo-1,2,3,4-tetrahydro-5-pyrimidinecarboxylate.

Methyl (4S)-3-{[(3-{4-[3-(Butyrylamino)Phenyl]-1-Piperidinyl}Propyl)Amino]Carbonyl}-4-(3,4-Difluorophenyl)-6-(Methoxymethyl)-2-oxo-1,2,3,4-Tetrahydro-5-Pyrimidinecarboxylate:

14.7 mg (65.5% yield) δ ¹H NMR (400 MHz, CDCl₃) δ 7.38 (s, 1H), 7.26 (s, 1H), 7.17-6.99 (m, 5H), 6.87 (s, 1H), 6.55 (s, 1H), 4.56 (ABq, 2H), 3.63 (s, 3H), 3.33 (s, 3H), 3.28-3.17 (m, 6H), 3.0 (br s, 2H), 2.51-2.36 (m, 3H), 2.25 (t, 2H, J=5.0 Hz), 2.10 (br s, 2H), 1.8-1.56 (m, 6H), 0.90 (t, 3H, J=5.0 Hz); ESMS m/e: 642.4 (M+H)⁺.

### EXAMPLE 45

(4R)-N-(3-{4-[3-(Butyrylamino)Phenyl]-1-Piperidinyl}Propyl)-4-(3,4-Difluorophenyl)-6-(Methoxymethyl)-2-oxo-1,2,3,4-Tetrahydro-5-Pyrimidinecarboxamide

Method:

(4R)-4-(3,4-difluorophenyl)-6-(methoxymethyl)-2-oxo-1,2,3,4-tetrahydro-5-pyrimidinecarboxylic acid:

A stirred mixture of one mole equivalent of methyl (4R)-4-(3,4-difluorophenyl)-6-(methoxymethyl)-2-oxo-1,2,3,4-tetrahydro-5-pyrimidinecarboxylate (10.0 g, 32.0 mmol) and lithium hydroxide (2 equivalents, 1.53 g, 64.0 mol) in H₂O-THF (2:1, 300 mL) was heated at reflux temperature for 1 h. The reaction mixture was concentrated, dissolved in water, washed with ethyl acetate and acidified (1 N HCl) to pH 3-4 (pH paper). The precipitated product was collected, washed with water and dried under reduced pressure to give the desired product in 90% yield.

(4R)-4-(3,4-Difluorophenyl)-6-(Methoxymethyl)-N-[3-(4-(3-Nitrophenyl)-3,6-Dihydro-1(2H)-Pyridinyl)Propyl]-2-oxo-1,2,3,4-Tetrahydro-5-Pyrimidinecarboxamide:

A solution of (4R)-4-(3,4-difluorophenyl)-6-(methoxymethyl)-2-oxo-1,2,3,4-tetrahydro-5-pyrimidinecarboxylic acid (1.2 eq), EDC (1.5 Eq.), N-methylmorpholine (2.0 Eq.) in dichloromethane was stirred at room temperature for 15 minutes, followed by addition of 3-(4-(3-nitrophenyl)-3,6-dihydro-1(2H)-pyridinyl)-1-propanamine (1.0 eq.) to the reaction mixture. The resulting solution was stirred for 18 hours, concentrated and chromatographed on silica to give (4R)-4-(3,4-difluorophenyl)-6-(methoxymethyl)-N-[3-(4-(3-nitrophenyl)-3,6-dihydro-1(2H)-pyridinyl)propyl]-2-oxo-1,2,3,4-tetrahydro-5-pyrimidinecarboxamide.

(4R)-N-{3-[4-(3-Aminophenyl)-1-Piperidinyl]Propyl}-4-(3,4-Difluorophenyl)-6-(Methoxymethyl)-2-oxo-1,2,3,4-Tetrahydro-5-Pyrimidinecarboxamide:

A mixture of (4R)-4-(3,4-difluorophenyl)-6-(methoxymethyl)-N-[3-(4-(3-nitrophenyl)-3,6-dihydro-1(2H)-pyridinyl)propyl]-2-oxo-1,2,3,4-tetrahydro-5-pyrimidinecarboxamide, 10% Pd/C in ethanol was hydrogenated (balloon method) for 2 days. The reaction mixture was filtered through Celite 545, washed with ethanol and concentrated to give the desired product.

(4R)-N-(3-{4-[3-(Butyrylamino)Phenyl]-1-Piperidinyl}Propyl)-4-(3,4-Difluorophenyl)-6-(Methoxymethyl)-2-oxo-1,2,3,4-Tetrahydro-5-Pyrimidinecarboxamide:

Into a 20 mL vial was added(4R)-N-{3-[4-(3-aminophenyl)-1-piperidinyl]propyl}-4-(3,4-difluorophenyl)-6-(methoxymethyl)-2-oxo-1,2,3,4-tetrahydro-5-pyrimidinecarboxamide (0.040 mmol), acid chloride (1.5 eq) and N,N-diisopropylethylamine (5.0 eq) in 2.0 mL of dichloromethane at room temperature. After 24 hrs, the reaction mixture was concentrated in vacuo and purified by preparative TLC (silica, 2000 microns, 95:5=dichloromethane:methanol with 1% of isopropylamine) to give 9.2 mg (45% yield) of the desired product: ¹H NMR (400 MHz, CD₃OD) δ 7.49 (s, 1H), 7.25 (d, 1H, J=7.6 Hz), 7.20-7.02 (m, 5H), 6.91 (d, 1H, J=8 Hz), 5.29 (s, 1H), 4.24 (ABq, 2H), 3.30 and 3.24 (two s, 3H), 3.46-3.12 (m, partially hidden by three s, 4H), 2.74 (br s, 4H), 2.25 (t, 2H, J=8.2 Hz), 2.04-1.69 (m, 7H), 1.63 (sextet, 2H, J=7.4 Hz), 0.91 (t, 3H, 7.4 Hz); ESMS m/e: 584.4 (M+H)⁺.

### EXAMPLE 46

The product was obtained according to the method described for (4R)-N-(3-{4-[3-(butyrylamino)phenyl]-1-piperidinyl}propyl)-4-(3,4-difluorophenyl)-6-(methoxymethyl)-2-oxo-1,2,3,4-tetrahydro-5-pyrimidinecarboxamide.

(4R)-4-(3,4-Difluorophenyl)-6-(Methoxymethyl)-2-oxo-N-(3-{4-[3-(Propionylamino)Phenyl]-1-Piperidinyl}Propyl)-1,2,3,4-Tetrahydro-5-Pyrimidinecarboxamide:

5.6 mg (24.6% yield); ¹H NMR (400 MHz, CD₃OD) δ 7.56 (s, 1H), 7.35 (d, 1H, J=6.9 Hz), 7.3-7.03 (m, 4H), 7.17 (br s, 1H), 6.99 (d, 1H, J=7.0 Hz), 5.45 (s, 1H), 4.33 (ABq, 2H), 3.41 (s, 3H), 3.37-3.23 (m, partially hidden, 4H), 2.8 (br s, 4H), 2.39 (d, 2H, J=9.3 Hz), 2.14-1.78 (m, 7H), 1.21 (t, 3H, J=7.6 Hz); ESMS m/e: 570.4 (M+H)⁺.

### EXAMPLE 47

The product was obtained according to the method described for (4R)-N-(3-{4-[3-(butyrylamino)phenyl]-1-piperidinyl}propyl)-4-(3,4-difluorophenyl)-6-(methoxymethyl)-2-oxo-1,2,3,4-tetrahydro-5-pyrimidinecarboxamide.

(4R)-4-(3,4-Difluorophenyl)-6-(Methoxymethyl)-N-[3-(4-{3-[(3-Methylbutanoyl)Amino]Phenyl}-1-Piperidinyl)Propyl]-2-oxo-1,2,3,4-Tetrahydro-5-Pyrimidinecarboxamide: 11.1 mg (46% yield); ¹H NMR (400 MHz, CD₃OD) δ 7.81 (d, 1H, J=8.5 Hz), 7.6 (s, 1H), 7.55 (s, 1H), 7.36 (br s, 1H), 7.31-7.17 (m, 3H), 7.01 (t, 1H, J=6.7 Hz) 6.64-6.61 (m, 1H), 5.45 (br s, 1H), 4.32 (ABq, 2H), 3.94 and 3.87 (two s, 3H), 3.42-3.12 (m, partially hidden, 2H), 3.1 (br s, 2H), 3.0 (t, 2H, J=11.1 Hz), 2.79-2.57 (m, 4H), 2.27-1.73 (m, 8H), 1.19 and 1.01 (two d, 6H, J=6.6 Hz); ESMS m/e: 598.4 (M+H)⁺.

### EXAMPLE 48

The product was obtained according to the method described for (4R)-N-(3-{4-[3-(butyrylamino)phenyl]-1-piperidinyl}propyl)-4-(3,4-difluorophenyl)-6-(methoxymethyl)-2-oxo-1,2,3,4-tetrahydro-5-pyrimidinecarboxamide.

(4R)-4-(3,4-Difluorophenyl)-6-(Methoxymethyl)-N-[3-(4-{3-[(2-Methylbutanoyl)Amino]Phenyl)-1-Piperidinyl)Propyl]-2-oxo-1,2,3,4-Tetrahydro-5-Pyrimidinecarboxamide:

6.7 mg (28% yield); ¹H NMR (400 MHz, CD₃OD) δ 7.59 (s, 1H), 7.35 (br s, 1H), 7.3-7.2 (m, 3H), 7.17 (br s, 1H), 7.01 (d, 1H, J=6.8 Hz), 5.45 (s, 1H), 4.33 (ABq, 2H), 3.39 (s, 3H), 3.29 (m, 2H), 2.84 (br s, 4H), 2.42 (m, 1H), 2.14-1.78 (m, 9H), 1.7 (m, 1H), 1.49 (m, 1H), 1.20 (d, 3H, J=6.7 Hz), 0.95 (t, 3H, J=6.6 Hz); ESMS m/e: 598.4 (M+H)⁺.

### EXAMPLE 49

The product was obtained according to the method described for (4R)-N-(3-{4-[3-(butyrylamino)phenyl]-1-piperidinyl}propyl)-4-(3,4-difluorophenyl)-6-(methoxymethyl)-2-oxo-1,2,3,4-tetrahydro-5-pyrimidinecarboxamide.

(4R)-4-(3,4-Difluorophenyl)-N-[3-(4-{3-[(3,3-Dimethylbutanoyl)Amino]Phenyl}-1-Piperidinyl)Propyl]-6-(Methoxymethyl)-2-oxo-1,2,3,4-Tetrahydro-5-Pyrimidinecarboxamide: 1.1 mg (4.4% yield); ¹H NMR (400 MHz, CD₃OD) δ 7.6-6.91 (m, 7H), 5.43 (s, 1H), 4.31 (ABq, 2H), 3.40 (s, 3H), 3.27-1.26 (m, 17H), 1.09 (s, 9H); ESMS m/e: 612.4 (M+H)⁺.

### EXAMPLE 50

The product was obtained according to the method described for (4R)-N-(3-{4-[3-(butyrylamino)phenyl]-1-piperidinyl}propyl)-4-(3,4-difluorophenyl)-6-(methoxymethyl)-2-oxo-1,2,3,4-tetrahydro-5-pyrimidinecarboxamide.

(4R)-4-(3,4-Difluorophenyl)-N-(3-{4-[3-(Isobutyrylamino)Phenyl]-1-Piperidinyl}Propyl)-6-(Methoxymethyl)-2-oxo-1,2,3,4-Tetrahydro-5-Pyrimidinecarboxamide:

12.7 mg (54% yield); ¹H NMR (400 MHz, CD₃OD) δ 7.59(s, 1H), 7.36 (d, 1H, J=8.6 Hz), 7.31-7.07 (m, 4H), 7.01 (d, 1H, J=6.5 Hz), 5.39 (s, 1H), 4.34 (ABq, 2H), 3.35 (s, 3H), 3.33-3.19 (m, partially hidden, 2H), 3.08-2.72 (m, 4H), 2.63 (t, 2H, J=7.2 Hz), 2.14-1.82 (m, 8H), 1.19 (d, 6H, J=6.9 Hz); ESMS m/e: 584.4 (M+H)⁺.

### EXAMPLE 51

The synthetic method is the same as described for the synthesis of (4S)-N-(3-{4-[3-(acetylamino)phenyl]-1-piperidinyl}propyl)-4-(3,5-difluorophenyl)-2-oxo-1,3-oxazolidine-3-carboxamide.

5-Acetyl-N-(3-{4-[3-(Acetylamino)Phenyl]-1-Piperidinyl}Propyl)-4-Methyl-2-oxo-6-(3,4,5-Trifluorophenyl)-3,6-Dihydro-1(2H)-Pyrimidinecarboxamide:

14.5 mg (46% yield); ¹H NMR (400 MHz, CDCl₃) δ 9.56 (s, 1H), 9.20 (s, 1H), 8.21 (s, 1H), 7.52 (s, 1H), 7.18 (t, 1H, J=7.8 Hz), 7.07-6.75 (m, 5H), 3.59-3.37 (m, 1H), 3.48-3.38 (m, 1H), 3.08 (br s, 2H), 2.57-2.39 (m, 5H), 2.25 (s, 3H), 2.21 (s, 3H), 2.19-1.59 (m, 9H); ESMS m/e: 586.3 (M+H)⁺; Anal. Calc. for C₃₀H₃₄F₃N₅O₄+0.1CHCl₃: C, 60.50; H, 5.75; N, 11.72. Found: C, 60.59; H, 5.40; N, 11.73.

### EXAMPLE 52

The synthetic method is the same as described for the synthesis of (4S)-N-(3-{4-[3-(acetylamino)phenyl]-1-piperidinyl}propyl)-4-(3,5-difluorophenyl)-2-oxo-1,3-oxazolidine-3-carboxamide.

Benzyl 3-{[(3-{4-[3-(Acetylamino)Phenyl]-1-Piperidinyl}Propyl)Amino]Carbonyl}-4-(2,4-Difluorophenyl)-6-Ethyl-2-oxo-1,2,3,4-Tetrahydro-5-Pyrimidinecarboxylate:

14.8 mg (41% yield); ¹H NMR (400 MHz, CDCl₃) δ 9.05 (br s, 1H), 8.14 (s, 1H), 7.47 (s, 1H), 7.37-7.21 (m, 8H), 7.18 (t, 1H, J=7.7 Hz), 6.94 (d, 1H, J=6.9 Hz), 6.87 (d, 1H, J=7.4 Hz), 6.7-6.62 (m, 3H), 5.09 (q, 2H, J=17.8 Hz), 3.48-3.24 (m, 2H), 3.04 (ABq, 2H), 2.88-2.71 (m, 2H), 2.52-2.39 (m, 2H), 2.19 (s, 3H), 2.17-1.88 (m, 3H), 1.77-1.58 (m, 3H), 1.19 (t, 3H, J=7.5 Hz); ESMS m/e: 674.4 (M+H)⁺.

### EXAMPLE 53

The synthetic method is the same as described for the synthesis of (4S)-N-(3-{4-[3-(acetylamino)phenyl]-1-piperidinyl}propyl)-4-(3,5-difluorophenyl)-2-oxo-1,3-oxazolidine-3-carboxamide.

N-(3-{4-[3-(Acetylamino)Phenyl]-1-Piperidinyl}Propyl)-4-(1,3-Benzodioxol-5-yl)-2,5-Dioxo-1,2,5,7-Tetrahydrofuro[3,4-D]Pyrimidine-3(4H)-Carboxamide:

8.75 mg (28% yield); ¹H NMR (400 MHz, CDCl₃) δ 9.81 (s, 1H), 8.14 (s, 1H), 7.53 (s, 1H). 7.21 (t, 1H, J=7.7 Hz), 6.99 (d, 1H, J=7.7 Hz), 6.91-6.7 (m, 4H), 6.42 (s, 1H), 5.9 (s, 2H), 4.75 (s, 2H), 3.61-3.5 (m, 1H), 3.37-3.27 (m, 1H), 3.08 (br s, 2H), 2.56-2.40 (m, 3H), 2.18 (s, 3H), 2.16-1.85 (m, 4H), 1.78-1.6 (m, 5H); ESMS m/e: 576.3 (M +H)⁺.

### EXAMPLE 54

The synthetic method is the same as described for the synthesis of (4S)-N-(3-{4-[3-(acetylamino)phenyl]-1-piperidinyl}propyl)-4-(3,5-difluorophenyl)-2-oxo-1,3-oxazolidine-3-carboxamide.

Methyl 1-{[(3-{4-[3-(Acetylamino)Phenyl]-1-Piperidinyl}Propyl)Amino]Carbonyl}-2-[(4-Methoxybenzyl)Sulfanyl]-4-Methyl-6-(4-Nitrophenyl)-1,6-Dihydro-5-Pyrimidinecarboxylate: 10.1 mg (26% yield); ¹H NMR (400 MHz, CDCl₃) δ 8.02 (d, 2H, J=7.5 Hz), 7.53 (br s, 1H), 7.44-7.27 (m, 6H), 7.14 (d, 2H, J=8.5 Hz), 6.99 (d, 1H, J=7.6 Hz), 6.75 (d, 2H, J=8.5 Hz), 6.2 (s, 1H), 4.23 (ABq, 2H), 3.78 (s, 3H), 3.7 (s, 3H), 3.58-3.48 (m, 1H) 3.37-3.26 (m, 2H), 3.04 (m, 2H), 2.61-2.43 (m, 3H), 2.41 (s, 3H), 2.16 (s, 3H), 2.15-1.64 (m, 8H); ESMS m/e: 729.3 (M+H)⁺.

### EXAMPLE 55

The synthetic method is the same as described for the synthesis of (4S)-N-(3-{4-[3-(acetylamino)phenyl]-1-piperidinyl}propyl)-4-(3,5-difluorophenyl)-2-oxo-1,3-oxazolidine-3-carboxamide .

N-(3-(4-[3-(Acetylamino) Phenyl]-1-Piperidinyl}Propyl)-4-(2,1,3-Benzoxadiazol-5-yl)-2,5-Dioxo-1,2,5,7-Tetrahydrofuro[3,4-D]Pyrimidine-3 (4H)-Carboxamide:

7.7 mg (12% yield); 1H NMR (400 MHz, CDCl₃) δ 7.97-6.83 (m, 7H), 6.49 (s, 1H), 5.51(s, 1H), 3.43-2.02 (m, 17H), 1.82 (s, 3H); ESMS m/e: 574.3 (M+H)⁺.

### EXAMPLE 56

The synthetic method is the same as described for the synthesis of (4S)-N-(3-{4-[3-(acetylamino)phenyl]-1-piperidinyl}propyl)-4-(3,5-difluorophenyl)-2-oxo-1,3-oxazolidine-3-carboxamide.

Methyl (4S)-3-{[(3-{4-[3-(Acetylamino)Phenyl]-1-Piperidinyl}Propyl)Amino]Carbonyl}-4-(3,4-Difluorophenyl)-6-Methyl-2-oxo-1,2,3,4-Tetrahydro-5-Pyrimidinecarboxylate:

16.6 mg (52% yield); ¹H NMR (400 MHz, CDCl₃) δ 9.55 (br s, 1H), 9.07 (s, 1H), 8.19 (s, 1H), 7.54 (s, 1H), 7.25-6.98 (m, 4H), 6.95 (d, 1H, J=8.0 Hz), 6.81 (d, 1H, J=7.5 Hz), 6.69 (s, 1H), 3.70 (s, 3H), 3.57-3.34 (m, 2H), 3.06 (t, 2H, J=11.6 Hz), 2.47 (t, 2H, J=8.1 Hz), 2.42 (s, 3H), 2.20 (s, 3H), 2.18-1.61 (m, 9H); ESMS m/e: 584.3 (M+H)⁺; Anal. Calc. for C₃₀H₃₅F₂N₅O+0.25CHCl₃: C, 59.23; H, 5.79; N, 11.42. Found: C, 59.61; H, 5.31; N, 11.48.

Peptide Synthesis:

Abbreviations: Fmoc: 9-Fluorenyloxycarbonyl-; Trityl: triphenylmethyl-; tBu-: tertiary butyl ester; OtBu-: tertiary butyl ether; Ng: N-guanidinyl; Nin: N-Indole; MBHA : methylbenzhydlamine; DMF: N,N-dimethylformamide; NMP: N-Methylpyrrolidinone; DIEA: diisopripylethyl amine; TFA: trifluoroacetic acid.

Small scale peptide syntheses were performed either manually, by using a sintered glass column with argon pressure to remove solvents and reagents, or by using an Advanced ChemTech 396-9000 automated peptide synthesizer (Advanced ChemTech, Louisville, Ky.). Large scale peptide syntheses were performed on a CS Bio 536 (CS Bio Inc., San Carlos, Calif.). Fmoc-Alanine-OH, Fmoc-Cysteine(Trityl)-OH, Fmoc-Aspartic acid(tBu)-OH, Fmoc-Glutamic acid(tBu)-OH, Fmoc-Phenylalanine-OH, Fmoc-Glycine-OH, Fmoc-Histidine(Trityl)-OH, Fmoc-Isoleucine-OH, Fmoc-Lysine(Boc)-OH, Fmoc-Leucine-OH, Fmoc-Methionine-OH, Fmoc-Asparagine(Trityl)-OH, Fmoc-Proline-OH, Fmoc-Glutamine(Trityl)-OH, Fmoc-Arginine(Ng-2,2,4,6,7 -Pentamethyldihydrobenzofuran-5-sulfonyl)-OH, Fmoc-Serine(OtBu-OH, Fmoc-Threonine(OtBu)-OH, Fmoc-Valine-OH, Fmoc-Tryptophan(NinBoc)-OH, Fmoc-Tyrosine(OtBu)-OH, Fmoc-Cyclohexylalanine-OH, and Fmoc-Norleucine , Fmoc-O-benzyl-phosphotyrosine were used as protected amino acids. Any corresponding D-amino acids had the same side-chain protecting groups, with the exception of Fmoc-D-Arginine, which had a Ng-2,2,5,7,8-pentamethylchroman-6-sulfonyl protecting group.

Peptides with C-terminal amides were synthesized on solid phase using Rink amide-MBHA resin. The Fmoc group of the Rink Amide MBHA resin was removed by treatment with 30% piperidine in DMF for 5 and 30 minutes respectively. After washing with DMF (3 times), methanol (2 times) and DMF/NMP (3 times), the appropriate Fmoc-protected amino acid (4 eq.) was coupled for 2 hours with HBTU or HATU (4 eq.) as the activating agent and DIEA (8eq.) as the base. In manual syntheses, the ninhydrin test was used to test for complete coupling of the amino acids. The Fmoc groups were removed by treatment with 30% piperidine in DMF for 5 and 30 minutes respectively. After washing with DMF (3 times), methanol (2 times) and DMF/NMP (3 times), the next Fmoc-protected amino acid (4 eq.) was coupled for 2 hours with HBTU or HATU (4eq.) as the activating agent and DIEA (8eq.) as the base. This process of coupling and deprotection of the Fmoc group was continued until the desired peptide was assembled on the resin. The N-terminal Fmoc group was removed by treatment with 30% piperidine in DMF for 5 and 30 minutes respectively. After washing with DMF (3 times), methanol (2 times), the resin(s) was vacuum dried for 2 hours. Cleavage of the peptide-on-resin and removal of the side chain protecting groups was achieved by treating with TFA:ethanedithiol:thioanisole: m-cresol:water:triisopropylsilane:phenol, 78/5/3/3/3/5/3 (5 mL per 100 mg resin) for 2.5-3 hours. The cleavage cocktail containing the peptide was filtered into a round bottom flask and the volatile liquids were removed by rotary evaporation at 30-40° C. The peptides were precipitated with anhydrous ether, collected on a medium-pore sintered glass funnel by vacuum filtration, washed with ether and vacuum dried.

Peptides with C-terminal acids were synthesized using 2-chlorotrityl chloride resin. The first amino acid was attached to the resin by dissolving 0.6-1.2eq. of the appropriate Fmoc-protected amino acid described above in dichloromethane (a minimal amount of DMF was added to facilitate the dissolution, if necessary). To this was added DIEA (4 eq. Relative to the Fmoc-amino acid) and the solution was added to the resin and shaken for 30-120 minutes. The solvents and the excess reagents were drained and the resin was washed with dichloromethane/methanol/DIEA (17/2/1) (3 times), dichloromethane (3 times), DMF (2 times), dichloromethane (2 times), and vacuum dried. The process of deprotection of the Fmoc group and coupling the appropriate Fmoc-protected amino acid was continued as described above, until the desired, fully protected peptide was assembled on the resin. The process for removal of the final Fmoc group and the cleavage and deprotection of the peptides was the same as described above for the peptides with C-terminal amides.

Purification of the peptides was achieved by preparative high performance column chromatography (HPLC), using a reverse-phase C-18 column (25×250 mm) (Primesphere or Vydac) with a gradient of acetonitrile (0.1% TFA) in water (0.1% TFA). The general gradient was from 10%-90% acetonitrile in water over 40 minutes. The fractions corresponding to each peak on the HPLC trace was collected, freeze dried and analyzed by electrospray mass spectrometery. The fraction having the correct mass spectral data corresponding to the desired peptide was then further analyzed by amino acid analysis, if necessary. All purified peptides were tested for homogeneity by analytical HPLC using conditions similar to that described above, but by using a 2.5×250 mm analytical column, and generally were found to have >95% purity.

### REFERENCES

See our published dihydropyrimidinone and oxazolidinone patents as references for the synthesis of the templates and the piperidines.

Also, for the synthesis of the aminopropyl piperidines and the templates, see:

Lagu, Bharat, et al., Design and synthesis of novel α₁ₐ adrenoceptor-selective antagonists. 3. Approaches to eliminate opioid agonist metabolites by using substituted phenylpiperazine side chains. J. Med. Chem. (1999), 42(23), 4794-4803. CODEN: JMCMAR ISSN:0022-2623. CAN 132:78527 AN 1999:680975 CAPLUS

Dhar, T. G. Murali, et al., Design and Synthesis of Novel α₁ₐ Adrenoceptor-Selective Antagonists. 2. Approaches To Eliminate Opioid Agonist Metabolites via Modification of Linker and 4-Methoxycarbonyl-4-phenylpiperidine Moiety. J. Med. Chem. (1999), 42(23), 4778-4793. CODEN: JMCMAR ISSN:0022-2623. CAN 132:18483 AN 1999:680971 CAPLUS

Nagarathnam, Dhanapalan, et al., Design and Synthesis of Novel α₁ₐ Adrenoceptor-Selective Antagonists. 1. Structure-Activity Relationship in Dihydropyrimidinones. J. Med. Chem. (1999), 42(23), 4764-4777. CODEN: JMCMAR ISSN:0022-2623. CAN 132:18482 AN 1999:680967 CAPLUS

Wong, Wai C., et al., Design and Synthesis of Novel α₁ₐ Adrenoceptor-Selective Antagonists. 4. Structure-Activity Relationship in the Dihydropyrimidine Series. J. Med. Chem. (1999), 42(23), 4804-4813. CODEN: JMCMAR ISSN:0022-2623. CAN 132:30317 AN 1999:680947 CAPLUS

Marzabadi, Mohammad R., et al., Design and synthesis of novel dihydropyridine alpha-lA antagonists. Bioorg. Med. Chem. Lett. (1999), 9 (19), 2843-2848. CODEN: BMCLE8 ISSN:0960-894X. CAN 132:44482 AN 1999:662323 CAPLUS

Wong, Wai C., et al., Alpha-1a adrenoceptor selective antagonists as novel agents for treating benign prostatic hyperplasia. Book of Abstracts, 217th ACS National Meeting, Anaheim, Calif., March 21-25 (1999), MEDI-156. CODEN: 67GHA6 AN 1999:92669 CAPLUS

Nagarathnam, D., et al., Design, synthesis and evaluation of dihydropyrimidinones as alpha-1a selective antagonists: 7. Modification of the piperidine moiety into 4-aminocyclohexane; identification and structure-activity relationship of SNAP 6991 analogs. Book of Abstracts, 217th ACS National Meeting, Anaheim, Calif., March 21-25 (1999), MEDI-110. CODEN: 67GHA6 AN 1999:92624 CAPLUS

Lagu, Bharat, et al., Heterocyclic substituted oxazolidinones for use as selective antagonists for human a 1A receptors. PCT Int. Appl. (1998), 258 pp. CODEN: PIXXD2 WO 9857940 A1 19981223 CAN 130:81508 AN 1999:9823 CAPLUS

Wong, Wai C., et al., Preparation of piperidinylpropylaminocarbonyldihydropyrimidones and related compounds as selective adrenergic a 1A receptor antagonists. PCT Int. Appl. (1998), 314 pp. CODEN: PIXXD2 WO 9851311 A2 19981119 CAN 130:25077 AN 1998:764290 CAPLUS

Nagarathnam, Dhanapalan, et al., Design and synthesis of novel α₁ₐ adrenoceptor-selective dihydropyridine antagonists for the treatment of benign prostatic hyperplasia. J. Med. Chem. (1998), 41(26), 5320-5333. CODEN: JMCMAR ISSN:0022-2623. CAN 130:110137 AN 1998:742998 CAPLUS

For the general procedure for Pd coupling of vinyl triflate and bononic acids or tributyl tin reagents: See, Wuston, Wise Synthesis 1991, 993)

(For Typical References, See:Schroeter, G. Ber. (1909) 42, 3356; and Allen, C. F. H.; Bell, A. Org. Syn. Coll. Vol. 3, (1955) 846).

For the preparation of the ether N-[4-(benzo-4′,5 [H]-furanpiperidine refer to W. E.Parham et al, J. Org. Chem. (1976) 41, 2268.

For the preparation of the ether piperidine precursor of Example 20, refer to W. E.Parham et al, J. Org. Chem. (1976) 41, 2268.

For the preparation of the indane piperidine precursor of Example 21, refer to M. S.Chambers J. Med. Chem. (1992) 35, 2033.

For the preparation of the piperidine precursor of Example 23, (K.Hashigaki et al. Chem.Pharm.Bull. (1984) 32, 3568.)

For the preparation of the piperidine precursor of Example 32, spiro[1H-indane-1,4′-piperidine], refer to M.S.Chambers et al. J. Med. Chem. (1992) 35, 2033.)

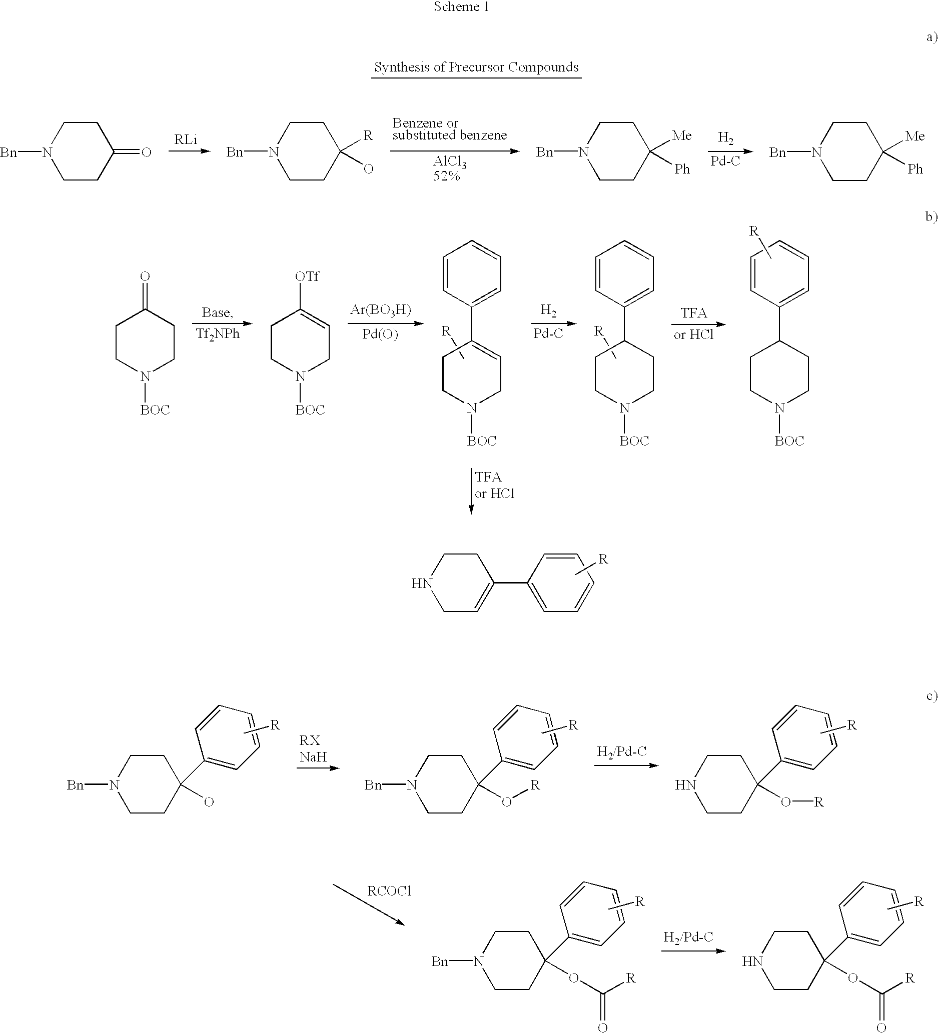

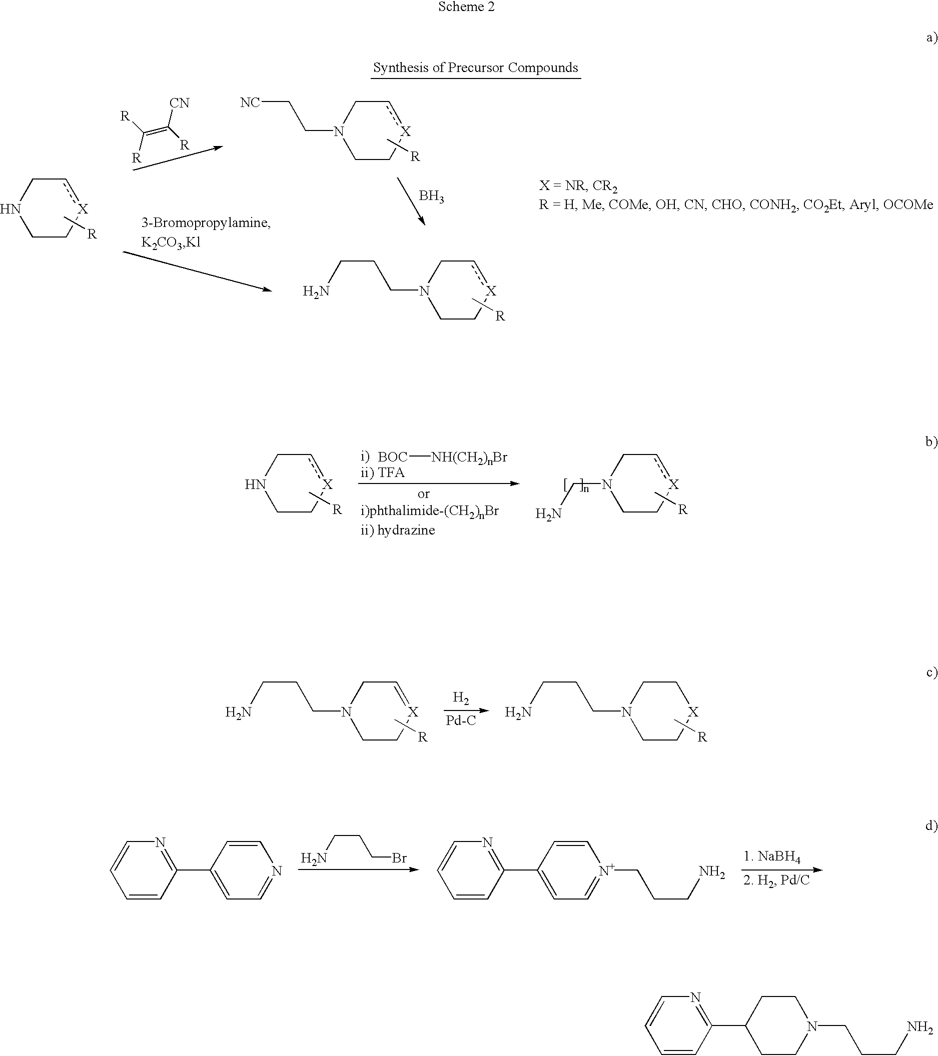

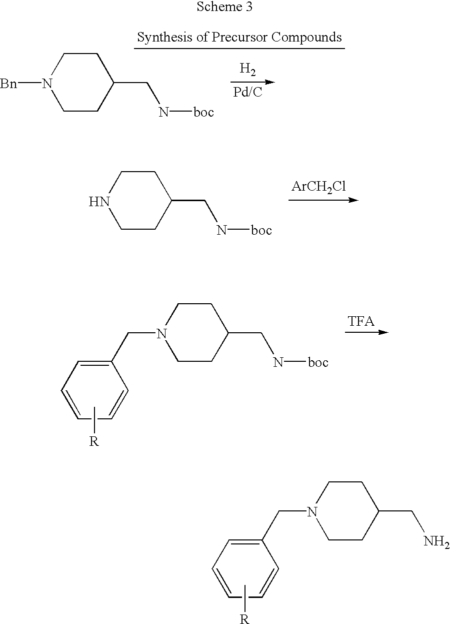

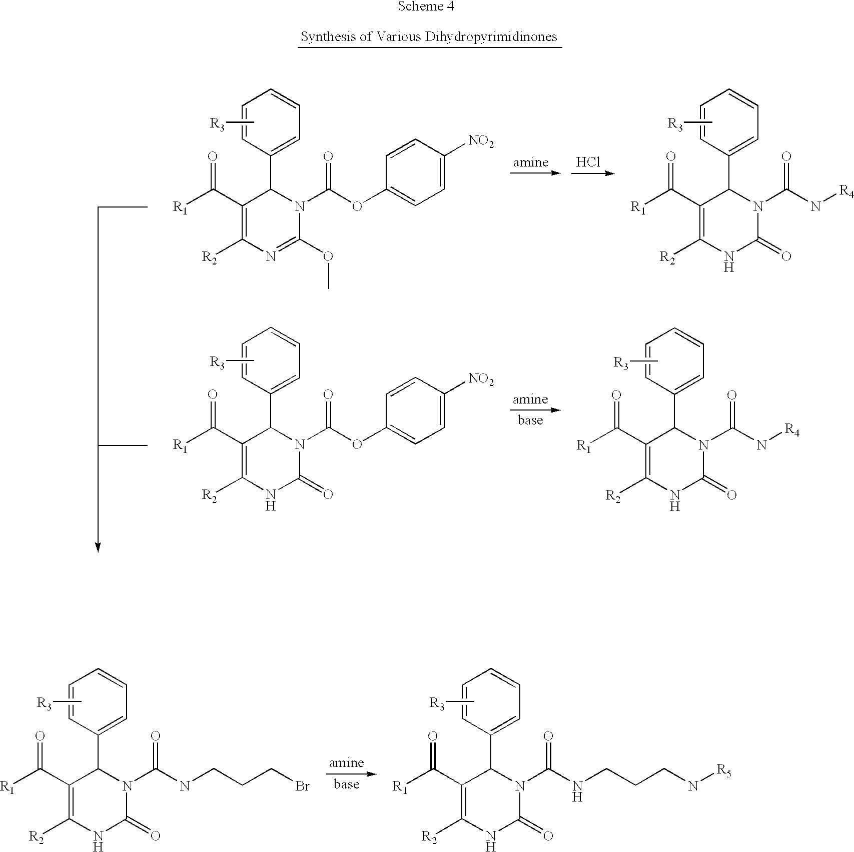

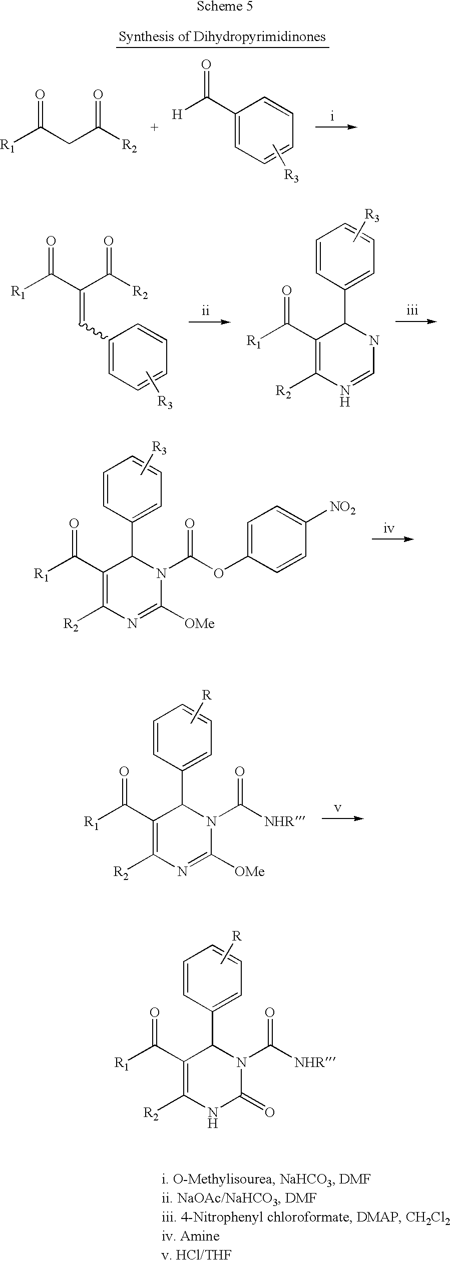

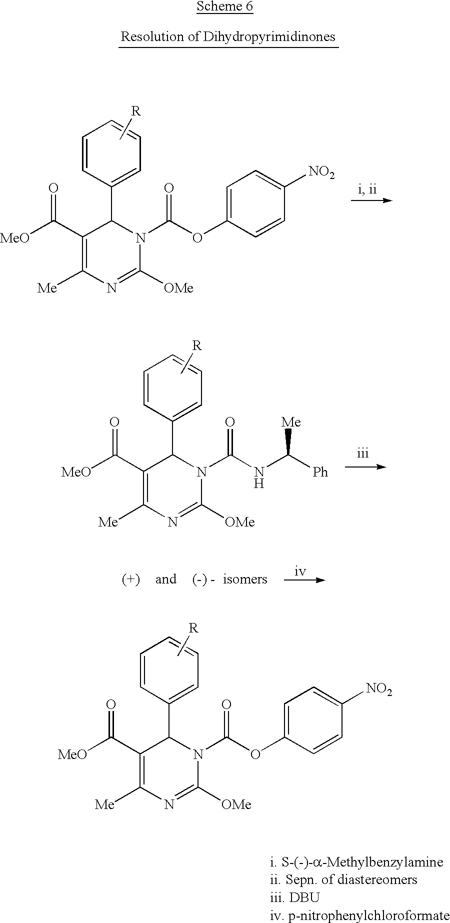

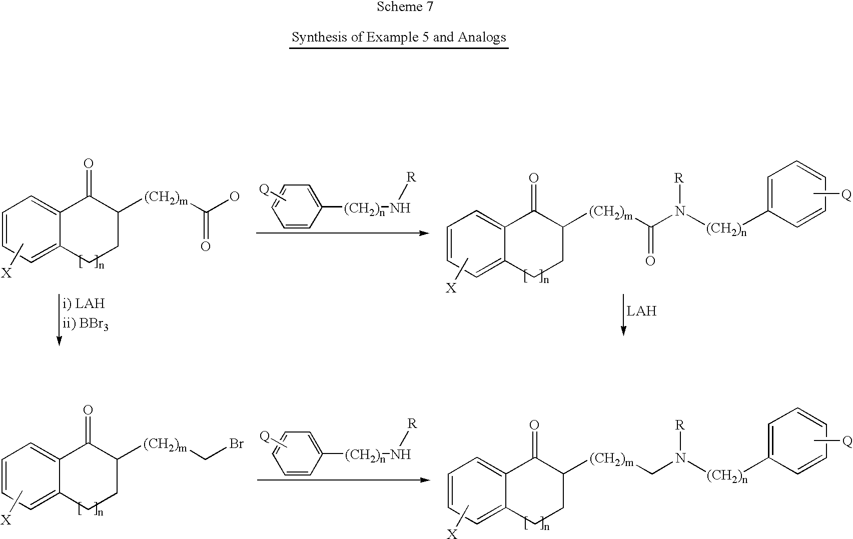

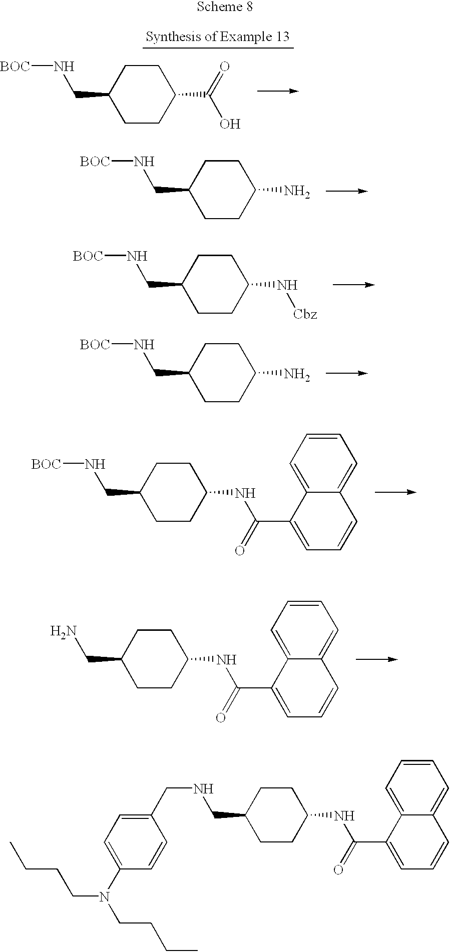

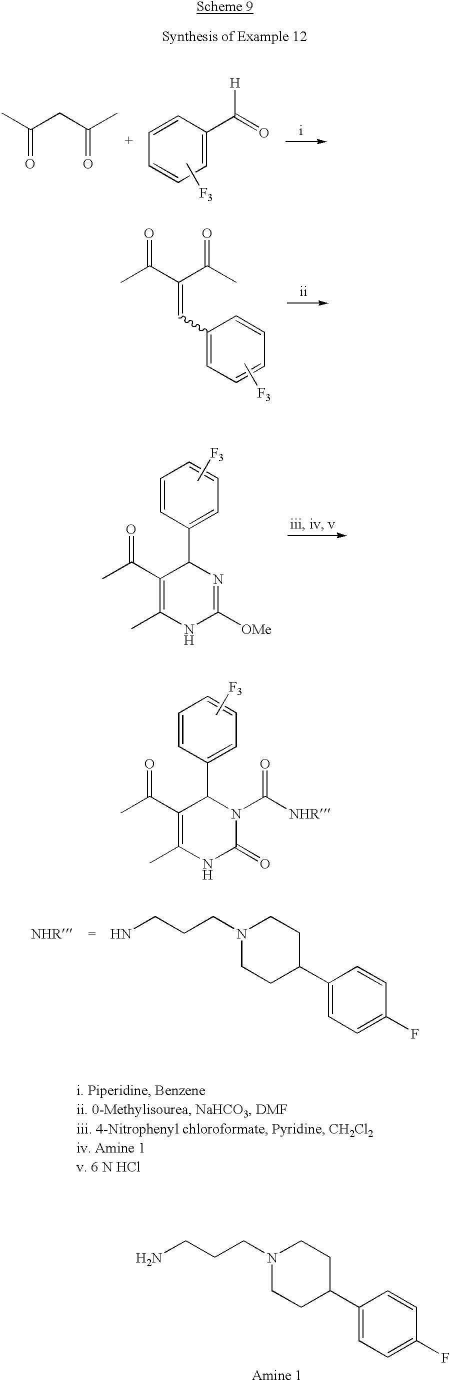

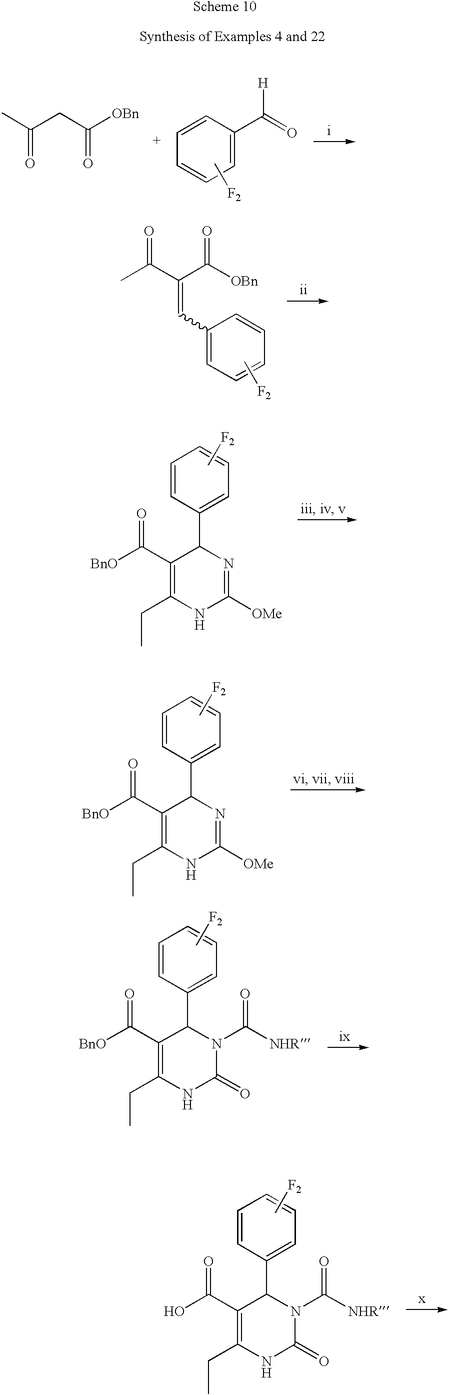

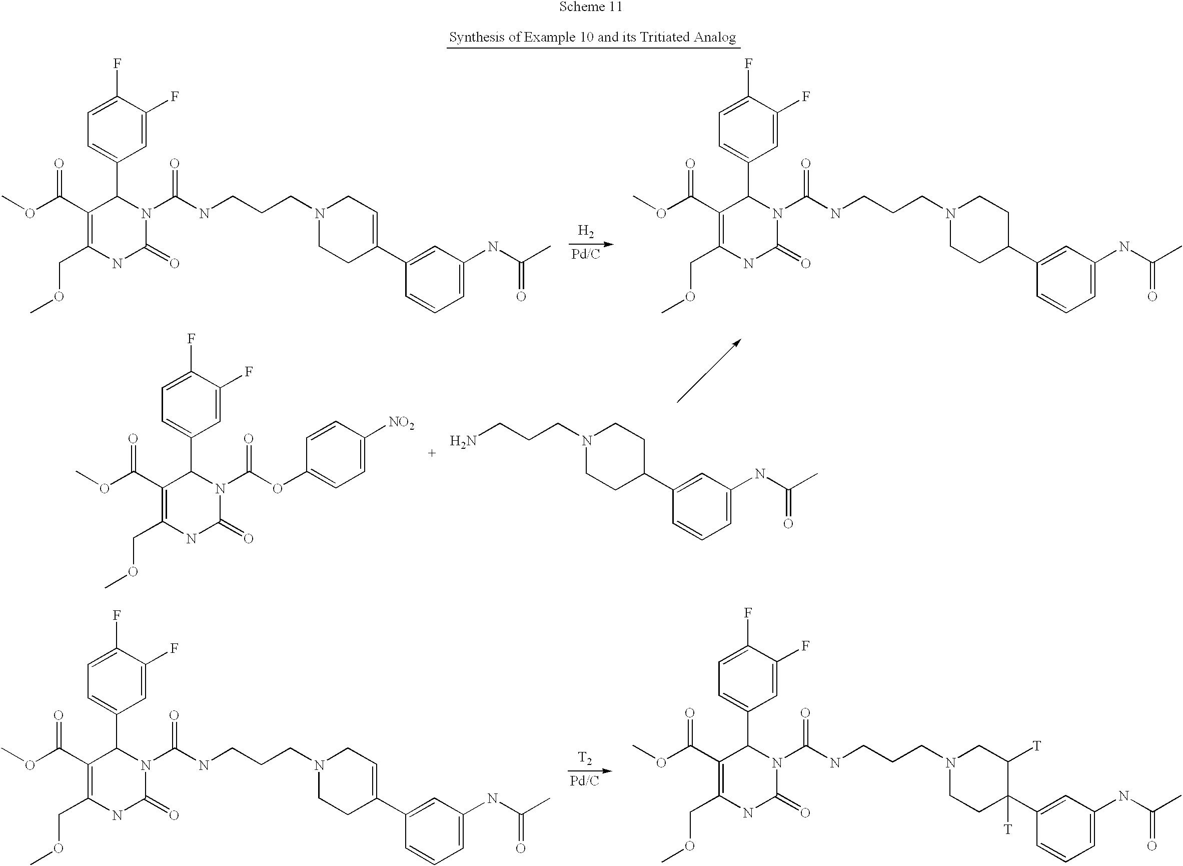

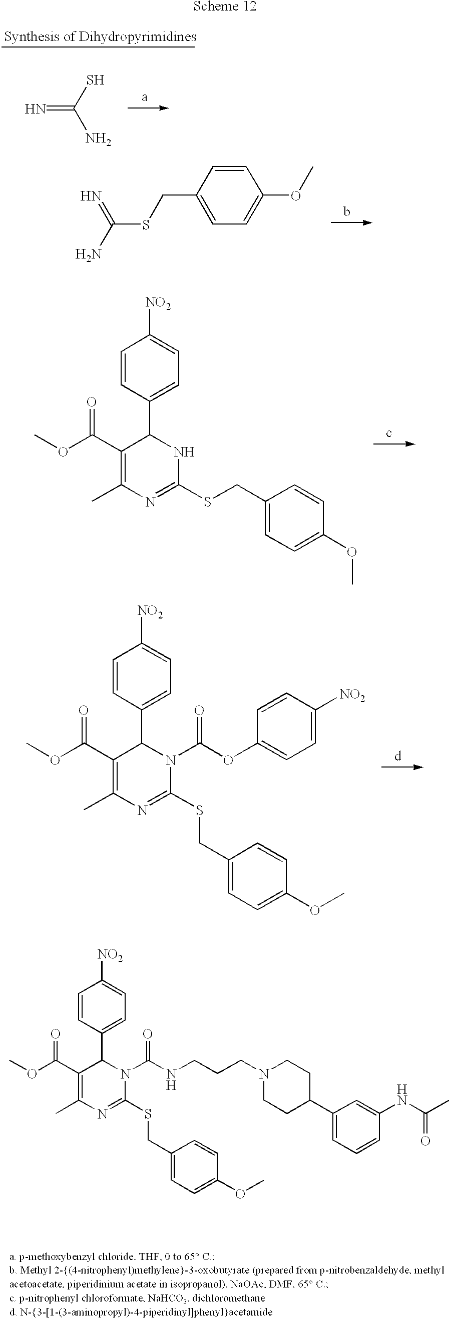

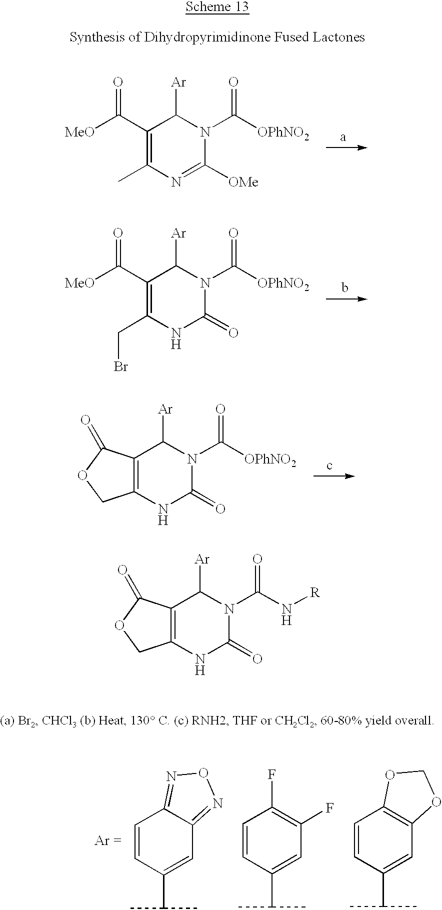

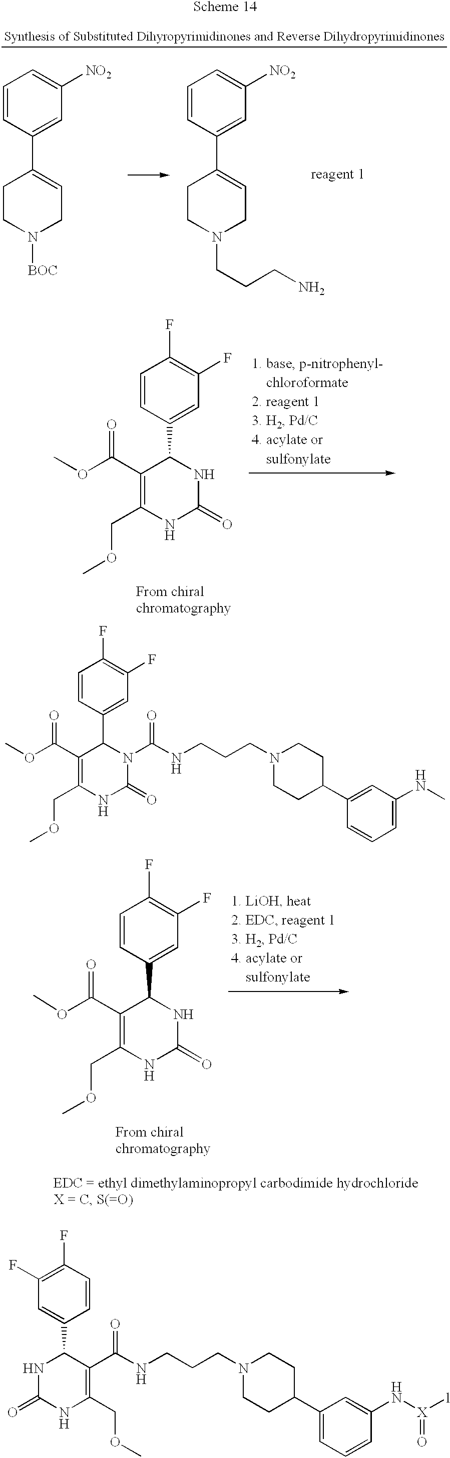

TABLE 1

| | | Kb (nM) |
|---|---|---|
| EXAMPLE No. | STRUCTURE | hMCH1 |
| 1 | 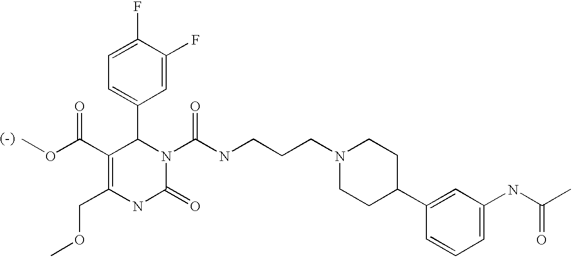 | 42 |
| 2 | 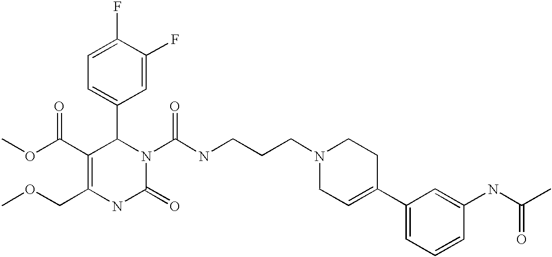 | 18 |
| 3 | 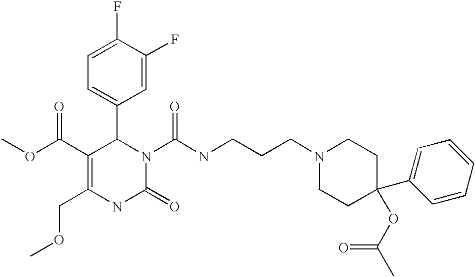 | 201 |
| 4 | 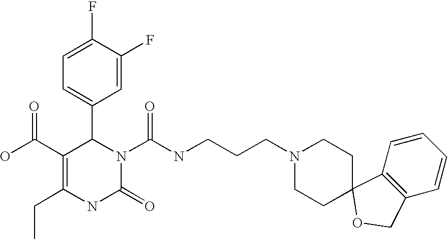 | 187 |
| 5 | 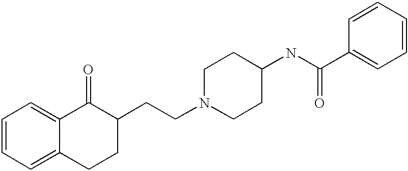 | 258 |
| 6 | 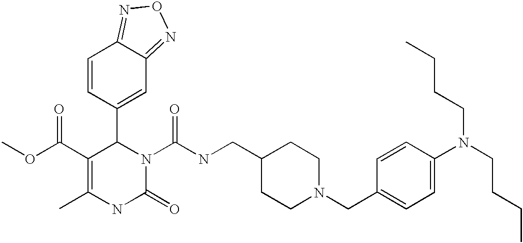 | 42 |
| 7 | 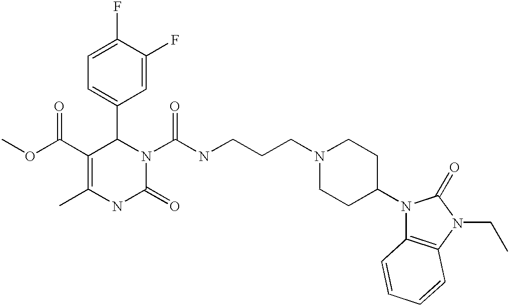 | 41 |
| 8 | 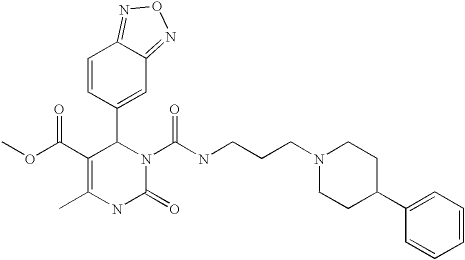 | 88 |
| 9 | 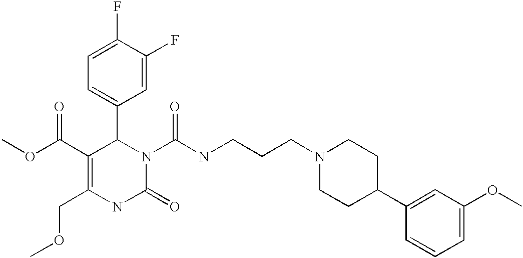 | 35 |
| 10 | 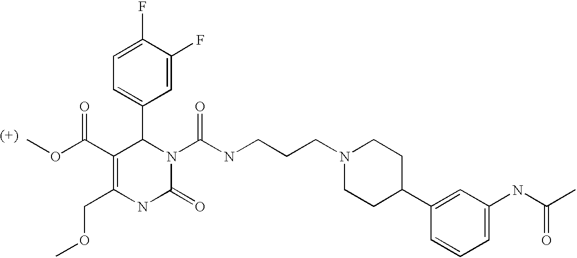 | 0.3 |
| 11 | 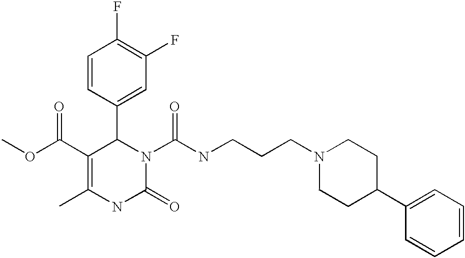 | 331 |
| 12 | 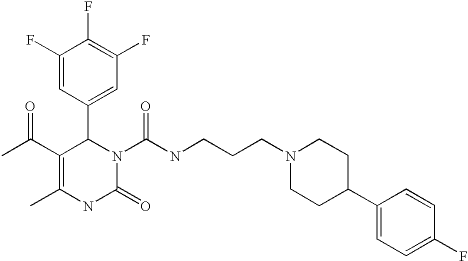 | 29 |
| 13 | 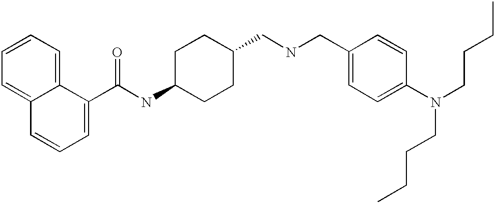 | 284 |
| 14 | 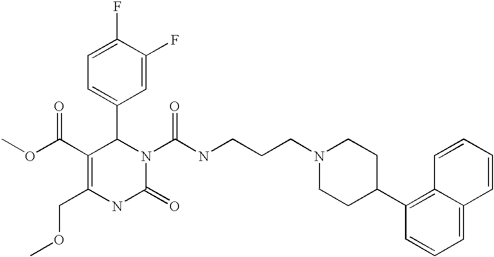 | 2 |
| 15 | 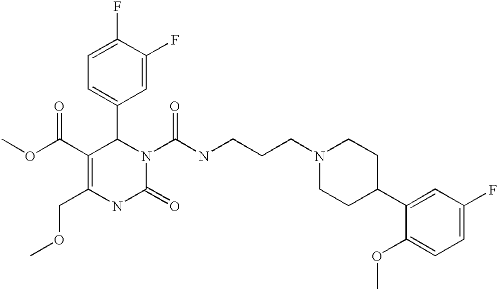 | 289 |
| 16 | 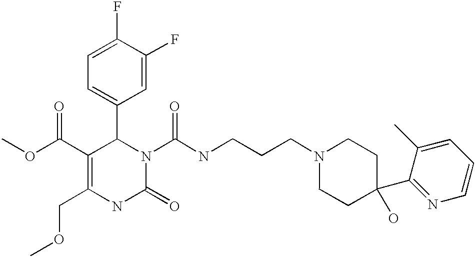 | 329 |
| 17 | 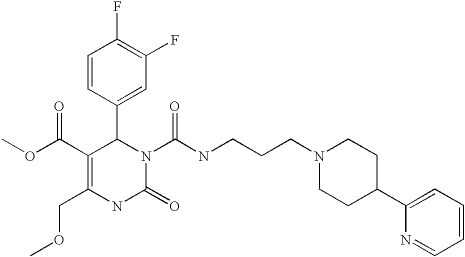 | 373 |
| 18 | 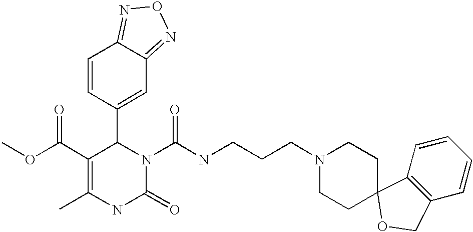 | 1 |
| 19 | 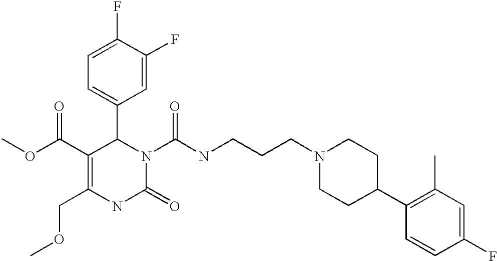 | 7 |
| 20 | 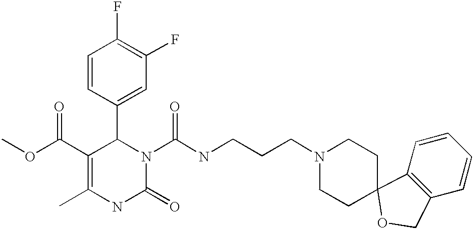 | 5 |
| 21 | 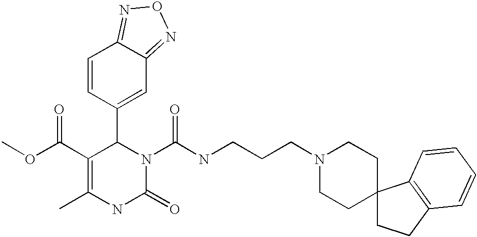 | 28 |
| 22 | 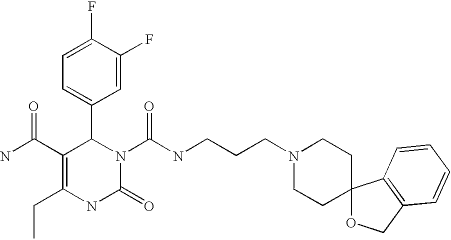 | 40 |
| 23 | 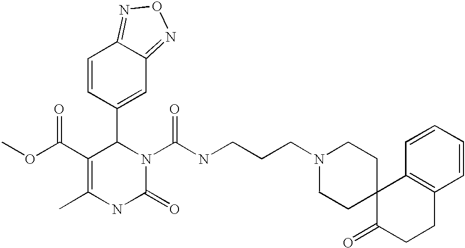 | 68 |
| 24 | 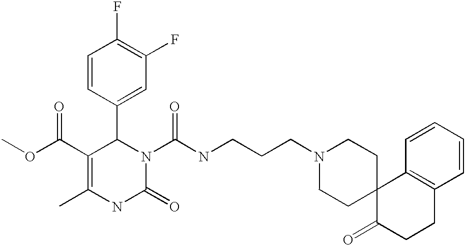 | 102 |
| 25 | 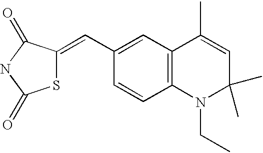 | 126 |
| 26 | 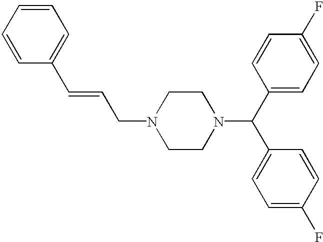 | 260 |
| 27 | 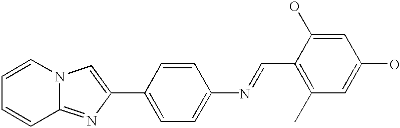 | 279 |
| 28 | 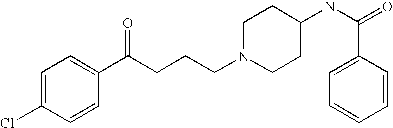 | 60 |
| 29 | 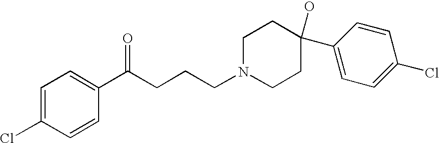 | 9 |
| 30 | 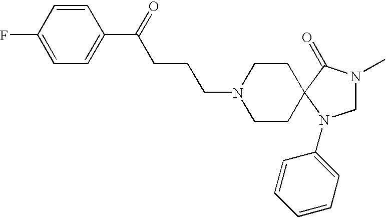 | 479 |
| 31 | 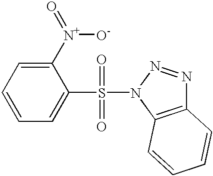 | 7 |
| 32 | 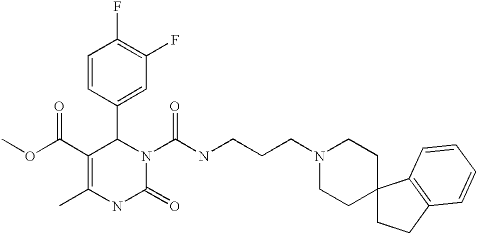 | 67 |
| 33 | 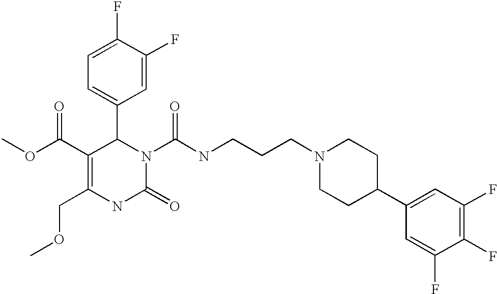 | 12 |
| 34 | 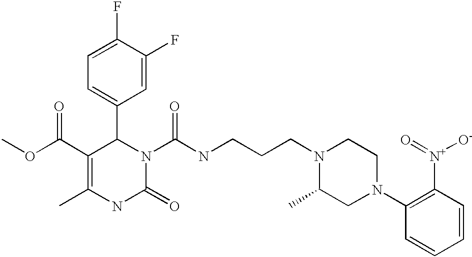 | 182 |
| 35 | 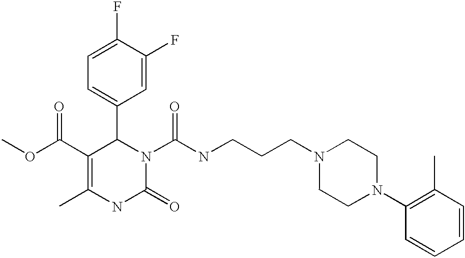 | 276 |
| 36 | 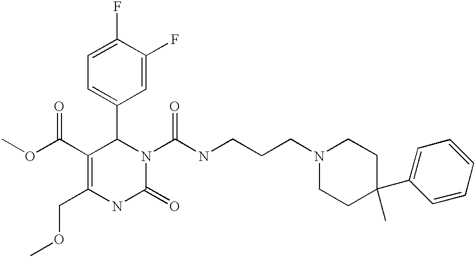 | 406 |
| 37 | 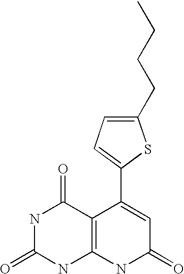 | 162 |

General Methods:

All reactions (except for those done by parallel synthesis reaction arrays) were performed under an Argon atmosphere and the reagents, neat or in appropriate solvents, were transferred to the reaction vessel via syringe and cannula techniques. The parallel synthesis reaction arrays were performed in vials (without an inert atmosphere) using J-KEM heating shakers (Saint Louis, Mo.). Anhydrous solvents were purchased from Aldrich Chemical Company and used as received. The examples described in the patent were named using ACD/Name program (version 2.51, Advanced Chemistry Development Inc., Toronto, Ontario, M5H2L3, Canada). Unless otherwise noted, the ¹H spectra were recorded at 300 and 400 MHz (QE Plus and Brüker respectively) with tetramethylsilane as internal standard. s=singlet; d=doublet; t=triplet; q=quartet; p=pentet; sext; sept; br=broad; m=multiplet. Elemental analyses were performed by Robertson Microlit Laboratories, Inc. Unless otherwise noted, mass spectra were obtained using low-resolution electrospray (ESMS) and MH⁺ is reported. Thin-layer chromatography (TLC) was carried out on glass plates precoated with silica gel 60 F₂₅₄ (0.25 mm, EM Separations Tech.). Preparative thin-layer chromatography was carried out on glass sheets precoated with silica gel GF (2 mm, Analtech). Flash column chromatography was performed on Merck silica gel 60 (230-400 mesh). Melting points (mp) were determined in open capillary tubes on a Mel-Temp apparatus and are uncorrected.

Piperidine Side Chain Intermediates

Tert-Butyl 4-{[(Trifluoromethyl)Sulfonyl]oxy}-1,2,3,6-Tetrahydro-1-Pyridinecarboxylate:

n-Butyl lithium (17.6 mL, 44.2 mmol, 2.5 M in hexanes) was added to a solution of diisopropyl amine (96.2 mL, 44.2 mmol) in 40 mL of dry THF at 0° C. and stirred for 20 minutes. The reaction mixture was cooled to −78° C. and tert-butyl 4-oxo-1-piperidinecarboxylate (Aldrich Chemical Company, 40.0 mmol) in THF (40 mL) was added dropwise to the reaction mixture and stirred for 30 minutes. Tf₂NPh (42.0 mmol, 15.0 g) in THF (40 mL) was added dropwise to the reaction mixture and stirred at ° C. overnight. The reaction mixture was concentrated in vacuo, re-dissolved in hexanes:EtOAc (9:1), passed through a plug of alumina and the alumina plug was washed with hexanes:EtOAc (9:1). The combined extracts were concentrated to yield 16.5 g of the desired product that was contaminated with some starting Tf₂NPh. ¹H NMR (400 MHz, 400 MHz, CDCl₃) δ 5.77 (s, 1H), 4.05 (dm, 2H, J=3.0 Hz), 3.63 (t, 2H, J=5.7 Hz), 2.45 (m, 2H), 1.47 (s, 9H).

Tert-Butyl 4-[3-(Amino)Phenyl]-1,2,3,6-Tetrahydro-1-Pyridinecarboxylate:

A mixture of 2 M aqueous Na₂CO₃ solution (4.2 mL), tert-butyl 4-{[(trifluoromethyl)sulfonyl]oxy}-1,2,3,6-tetrahydro-1-pyridine-carboxylate (0.500 g, 1.51 mmol), 3-aminophenylboronic acid hemisulfate (0.393 g, 2.11 mmol), lithium chloride (0.191 g, 4.50 mmol) and tetrakis-triphenylphosphine palladium (0) (0.080 g, 0.075 mmol) in dimethoxyethane (5 mL) was heated at reflux temperature for 3 hours, under an inert atmosphere (an initial degassing of the mixture is recommended to prevent the formation of triphenylphosphine oxide). The organic layer of the cooled reaction mixture was separated and the aqueous layer was washed with ethyl acetate (3×). The combined organic extracts were dried and concentrated in vacuo. The crude product was chromatograghed (silica, hexanes:EtOAc:dichloromethane (6:1:1) with 1% added isopropylamine to protect the BOC group from hydrolysis) to give 0.330 g of the desired product in 81% yield: 1H NMR (400 MHz, CDCl₃) δ 7.12 (t, 1H, J=7.60 Hz), 6.78 (d, 1H, J=8.4 Hz), 6.69 (t, 1H, J=2.0 Hz), 6.59 (dd, 1H, J=2.2, 8.0 Hz), 6.01 (m, 1H), 4.10-4.01 (d, 2H, J=2.4 Hz), 3.61 (t, 2H, J=5.6 Hz), 2.52-2.46 (m, 2H), 1.49 (s, 9H); ESMS m/e: 275.2 (M+H)⁺.

Anal. Calc. for C₁₆H₂₄N₂O₂: C, 70.04; H, 8.08; N, 10.21. Found: C, 69.78; H, 7.80; N, 9.92.

Tert-Butyl 4-[3-(Amino)Phenyl]-1-Piperidinecarboxylate

A mixture of 3.10 g of tert-butyl 4-(3-aminophenyl)-1,2,3,6-tetrahydropyridine-1-carboxylate (11.3 mmol) and 1.0 g of 10% Pd/C in 200 mL of ethanol was hydrogenated at room temperature using the balloon method for 2 days. The reaction mixture was filtered and washed with ethanol. The combined ethanol extracts were concentrated in vacuo and the residue was chromatographed on silica (dichloromethane: methanol 95:5 with 1% isopropylamine added to protect the BOC group from hydrolysis) to give 2.63 g of the desired product (84%).

Tert-Butyl 4-[3-(Acetylamino)Phenyl]-1,2,3,6-Tetrahydro-1-Pyridinecarboxylate: A mixture of saturated of aqueous Na₂CO₃ solution (25 mL), tert-butyl 4-{[(trifluoromethyl)sulfonyl]oxy}-1,2,3,6-tetrahydro-1-pyridine-carboxylate (20 mmol), 3-acetamidophenylboronic acid (30 mmol) and tetrakis-triphenylphosphine palladium (0) (1.15 g) and dimethoxyethane (40 mL) was heated at reflux temperature overnight. The organic layer of the cooled reaction mixture was separated and the aqueous layer was washed with ethyl acetate (3×). The combined organic extracts were dried and concentrated in vacuo. The crude product was chromatograghed, giving the desired product: ¹H NMR (CDCl₃) δ 8.11 (br s, 1H), 7.57 (br s, 1H), 7.41 (br d , 1H, J=7.8 Hz), 7.25 (apparent t, 1H, J=7.8 Hz), 7.08 (br d, 1H, J=7.8 Hz), 5.99 (br s, 1H), 4.03 (br m, 2H, J=2.7 Hz), 3.59 (t, 2H, J=5.7 Hz), 2.46 (m, 2H,), 2.16 (s, 3H), 1.49 (s, 9H).

N1-13-(1,2,3,6-Tetrahydro-4-Pyridinyl)Phenyl]Acetamide:

A solution of 4 M HCl in dioxane (10 mL) was added to tert-butyl 4-[3-(acetylamino)phenyl]-1,2,3,6-tetrahydro-1-pyridinecarboxylate (8.25 mmol) in dichloromethane (30 mL). The reaction mixture was stirred at room temperature overnight, concentrated in vacuo, giving the desired product as the hydrochloride salt (2.1 g): ¹H NMR (CDCl₃) δ 7.41-7.00 (m, 4H), 6.10 (br, 1H), 3.55 (m, 2H), 3.16 (t, 2H, J=5.7 Hz), 2.44 (m, 2H), 2.19 (s, 3H).

Tert-Butyl N-(3-Bromopropyl)Carbamate:

Prepared from 3-bromopropylamine hydrobromide and BOC₂O in the presence of base in dichloromethane, 9.89 mmol: ¹H NMR (CDCl₃) δ 5.07 (br, 1H), 3.31 (t, 2H, J=6.6 Hz), 3.12 (apparent br q, 2H, J=6.0 Hz), 1.92 (p, 2H, J=6.6 Hz), 1.30 (s, 9H).

Tert-Butyl N-(3-{4-[3-(Acetylamino)Phenyl]-1,2,3,6-Tetrahydro-1-Pyridinyl}Propyl)Carbamate:

A solution of N1-[3-(1,2,3,6-tetrahydro-4-pyridinyl)phenyl]acetamide.HCl (8.24 mmol), tert-butyl N-(3-bromopropyl)carbamate and potassium carbonate (33 mmol) in dry dioxane (30 mL) was heated at reflux temperature overnight. The solids were removed by filtration, the solution was concentrated in vacuo and the product was chromatograghed, giving the desired product (110 mg).

Tert-Butyl N-(3-4-[3-(Acetylamino)Phenyl]-1,2,3,6-Tetrahydro-1-Pyridinylpropyl)Carbamate:

¹H NMR (CDCl₃) δ 7.65 (s, 1H), 6.98 (s, 1H), 7.45 (d, 1H, J=7.8 Hz), 7.16 (apparent t, 1H, J=7.8 Hz), 7.10 (d, 1H, J=7.8 Hz), 6.02 (s, 1H), 5.23 (b, 1H), 3.40 (b, 2H), 3.30-1.80 (m, 10H), 2.18 (s, 3H), 1.45 (s, 9H).

N1-{3-[1-(3-Aminopropyl)-1,2,3,6-Tetrahydro-4-Pyridinyl]Phenyl}Acetamide:

A 1:1 solution of TFA:CH₂Cl₂ (5 mL) was added to tert-butyl N-(3-{4-[3-(acetylamino)phenyl]-1,2,3,6-tetrahydro-1-pyridinyl}propel)carbamate in dichloromethane (5 mL). The resulting solution was stirred at room temperature for 1-3 days, saturated NaHCO₃ was added until pH>6, the organic layer was separated, and dried in vacuo, giving the desired product (45 mg):

N1-{3-[1-(3-Aminopropyl)-1,2,3,6-Tetrahydro-4-Pyridinyl]Phenyl}Acetamide:

From N1-{3-[1-(3-aminopropyl)-1,2,3,6-tetrahydro-4-pyridinyl]phenyl}acetamide and acid (TFA or HCl), followed by basification of the resulting salt: ¹H NMR (CDCl₃) δ 7.68 (br, 1H), 7.35 (dm, 1H, J=7.8 Hz), 7.25 (apparent t, 1H, J=7.8 Hz), 7.15 (dm, 1H, J=7.8 Hz), 6.12 (m, 1H), 3.22 (m, 2H), 3.03 (t, 2 H, J=7.3 Hz), 2.78 (t, 2H, J=5.5 Hz), 2.70-2.50 (m, 4H), 2.10 (s, 3H), 1.87 (p, 2H, J=7.3 Hz).

Tert-Butyl 4-[3-(Acetylamino)Phenyl]-1-Piperidinecarboxylate:

A mixture tert-butyl 4-[3-(acetylamino)phenyl]-1,2,3,6-tetrahydro-1-pyridinecarboxylate (710 mg) and 5% Pd/C (100 mg) in EtOH (10 mL) was hydrogenated (balloon technique) at room temperature overnight. The reaction mixture was passed through a pad of Celite 545 and the pad of Celite was washed with ethanol. The combined ethanol extracts were concentrated and chromatograghed, giving the desired product (660 mg) 1H NMR (CDCl₃) δ 7.80 (s, 1H), 7.41-7.20 (m, 3H), 6.94 (d, 1H, J=7.5 Hz), 4.21 (m, 2H), 2.75 (m, 2H), 2.62 (m, 1H), 2.16 (s, 3H), 1.78 (m, 2H), 1.56 (m, 2H), 1.48 (s, 9H)

N1-[3-(4-Piperidyl)Phenyl]Acetamide:

A solution of HCl in dioxane (4N, 5 mL) was added to tert-butyl 4-[3-(acetylamino)phenyl]-1-piperidinecarboxylate (660 mg) in dry dichloromethane (15 mL). The reaction mixture was stirred at room temperature overnight and concentrated in vacuo, giving the desired product (550 mg): mp 102-104° C.; ¹H NMR (CDCl₃) δ 2.02 (d, J=13.2 Hz, 2H), 2.11-2.45 (m, 5H), 2.67-2.77 (m, 1H), 3.00-3.10 (m, 2H), 3.51 (d, J=10.5 Hz, 2H), 6.94 (d, J=7.5 Hz, 1H), 7.20-7.46 (m, 3H), 7.60 (s, 1H); Anal. Calcd. For C₁₃H₁₉N₂OCl+0.86 CH₂Cl₂: C, 50.78; H, 6.37; N, 8.55. Found: C, 50.80; H, 7.55; N, 7.01.

Tert-Butyl N-(3-{4-[3-(Acetylamino)Phenyl]Piperidino}Propyl)Carbamate:

A solution of N1-[3-(4-piperidyl)phenyl]acetamide (550 mg, 0.210 mmol), tert-butyl N-(3-bromopropyl)carbamate (550 mg, 0.230 mmol), K₂CO₃ (1.10 g, 0.890 mmol), diisopropylethyl amine (1.50 mL) and a few crystals of KI in dioxane (20 mL) was heated at reflux temperature for 2 days. The precipitated salts were removed by filtration, concentrated in vacuo and the crude product was chromatographed, giving the desired product (340 mg): ¹H NMR (CDCl₃) δ 8.15 (s, 1H), 7.47-7.44 (m, 2H), 7.22 (t, 1 H, J=7.8 Hz), 6.94 (d, 1H, J=7.8 Hz), 5.53 (b, 1H), 3.23 (b, 6H), 2.80-1.60 (m, 9H), 2.20 (s, 3H), 1.45 (s, 9H).

N1-{3-[1-(3-Aminopropyl)-4-Piperidyl]Phenyl}Acetamide:

TFA (1.0 mL) was added to a solution of tert-butyl N-(3-{4-[3-(acetylamino)phenyl]piperidino}propyl)carbamate (340 mg) in dry dichloromethane (10 mL) and stirred at room temperature for 5 h. A 10% aqueous solution of KOH was added to the reaction mixture until pH>6 and then the dichloromethane was removed in vacuo. The aqueous layer was frozen and lyophilized to give a solid, which was extracted with methanol. Removal of the solvent gave the desired product (120 mg) as an oil: ¹H NMR (CDCl₃) δ 7.23-7.16 (apparent t, 1H, J=7.5 Hz), 6.95-6.92 (m, 1H), 3.03-2.99 (m, 2H), 2.77-2.73 (t, 2H, J=6.6 Hz), 2.50-1.60 (m, 10H), 2.13 (s, 3H).

Tert-Butyl 4-(3-Nitrophenyl)-3,6-Dihydro-1(2H)-Pyridinecarboxylate

¹H NMR (400 MHz, 400 MHz, CDCl₃) δ 8.23 (s, 1H), 8.11 (d, 1H, J=8.0 Hz), 7.69 (d, 1H, J=8.0 Hz), 7.51 (t, 1H, J=8.0 Hz), 6.20 (m, 1H), 4.17-4.08 (m, 2H), 3.67 (t, 2H, J=5.6 Hz), 2.61-2.52 (m, 2H), 1.50 (s, 9H); ESMS m/e: 249.1 (M+H−C₄H)⁺.

1,2,3,6-Tetrahydro-4-(3-Nitrophenyl)Pyridine:

Into a stirred solution of 5.00 g (16.0 mmol) of tert-butyl 1,2,3,6-tetrahydro-4-(3-nitrophenyl)pyridine-1-carboxylate in 100 ml of 1,4-dioxane at 0° C. was bubbled HCl gas for 10 minutes. The reaction mixture was allowed to warm to room temperature and the bubbling of the HCl gas was continued for an additional 1 hour. The solvent was removed in vacuo, the residue was dissolved in 50 mL of water and was neutralized by the addition of KOH pellets. The aqueous solution was extracted with 3×80 mL of dichloromethane and the combined organic extracts were dried (MgSO₄), filtered and concentrated in vacuo. The residue was purified by column chromatography (silica, 9:1, dichloromethane:methanol+1% isopropyl amine) to afford 2.85 g (87.5% yield) of the desired product: ¹H NMR (400 MHz, 400 MHz, CDCl₃) δ 8.24 (s, 1H), 8.09 (d, 1H, J=8.4 Hz), 7.71 (d, 1H, J=8.0 Hz), 7.49 (t, 1H, J=8.0 Hz), 6.35-6.25 (m, 1H), 3.58 (apparent q, 2H, J=3.0 Hz), 3.14 (t, 2H, J=5.6 Hz), 2.54-2.46 (m, 2H).

Tert-Butyl 3-(4-(3-Nitrophenyl)-3,6-Dihydro-1(2H)-Pyridinyl)Propylcarbamate:

A mixture of 2.80 g (14.0 mmol) of 1,2,3,6-tetrahydro-4-(3-nitrophenyl)pyridine, 3.60 g (15.0 mmol) of tert-butyl N-(3-bromopropyl)carbamate, 11.6 g (84.0 mmol) of K₂CO₃, 14.6 mL (84.0 mmol) of diisopropylethylamine and 0.78 g (2.00 mmol) of tetrabutylammonium iodide in 250 mL of 1,4-dioxane was heated at reflux temperature for 14 hours.

The reaction mixture was filtered and the filtrate was dried (MgSO₄), concentrated in vacuo and the residue was purified by column chromatography (silica, 9:1, dichloromethane: methanol+1% isopropyl amine) to afford 4.35 g (85.7% yield) of the desired product: ¹H NMR (400 MHz, 400 MHz, CDCl₃) δ 8.24 (t, 1H, J=1.9 Hz), 8.09 (dd, 1H, J=1.9, 8.0 Hz), 7.70 (apparent d, 1H, J=8.0 Hz), 7.49 (t, 1H, J=8.0 Hz), 6.23 (m, 1H), 3.29-3.18 (m, 4H), 2.75 (t, 2H, J=5.6 Hz), 2.64-2.54 (m, 4H), 1.82-1.70 (m, 2H), 1.44 (s, 9H); ESMS m/e: 362.2 (M+H)⁺.

3-(4-(3-Nitrophenyl)-3,6-Dihydro-1(2H)-Pyridinyl)-1-Propanamine:

Into a stirred solution of 4.35 (12.0 mmol) of tert-butyl 3-(4-(3-nitrophenyl)-3,6-dihydro-1(2H)-pyridinyl)propylcarbamate in 100 ml of 1,4-dioxane at 0° C. was bubbled HCl gas for 10 minutes. The reaction mixture was allowed to warm to room temperature and the bubbling was continued for an additional 1 hour. The solvent was removed in vacuo, the residue was dissolved in 50 mL of water and was neutralized by the addition of KOH pellets. The aqueous solution was extracted with 3×80 mL of dichloromethane, the combined organic extracts were dried (MgSO₄), filtered and concentrated in vacuo. The residue was purified by column chromatography (silica, 9:1, dichloromethane:methanol+1% isopropyl amine) to afford 3.05 g (97.0% yield) of the desired product: ¹H NMR (400 MHz, 400 MHz, CDCl₃) δ 8.24 (t, 1H, J=1.8 Hz), 8.09 (dd, 1H, J=1.8, 8.2 Hz), 7.69 (dd, 1H, J=1.8, 8.2 Hz), 7.48 (t, 1H, J=8.2 Hz), 6.24 (m, 1H), 3.21 (d, 2H, J=3.6 Hz), 2.84 (t, 2H, J=6.6 Hz), 2.75 (t, 2H, J=5.8 Hz), 2.64-2.54 (m, 4H), 1.76 (m, 2H); ESMS m/e: 262.2 (M+H)⁺; Anal. Calc. for C₁₄H₁₉N₃O₂ (0.06 CHCl₃): C, 62.90; H, 7.16; N, 15.65. Found: C, 63.20; H, 7.16; N, 15.65.

Methyl (4S)-3-[({3-[4-(3-Aminophenyl)-1-Piperidinyl]Propyl}Amino)Carbonyl]-4-(3,4-Difluorophenyl)-6-(Methoxymethyl)-2-oxo-1,2,3,4-Tetrahydro-5-Pyrimidinecarboxylate:

A mixture of 3.02 g (6.33 mmol) 5-methyl 1-(4-nitrophenyl) (6S)-6-(3,4-difluorophenyl)-4-(methoxymethyl)-2-oxo-3,6-dihydro-1,5(2H)-pyrimidinedicarboxylate, 1.50 g (5.80 mmol) of 3-(4-(3-nitrophenyl)-3,6-dihydro-1(2H)-pyridinyl)-1-propanamine, 7.94 g (75.5 mmol) of K₂CO₃ and 1.00 mL of methanol in 200 mL dichloromethane (under argon) was stirred at room temperature for 1 hour. The reaction mixture was filtered and concentrated in vacuo. The residue was dissolved in 100 mL of ethyl acetate and washed 3×50 mL of 5% aqueous NaOH solution, the organic layer was dried (MgSO₄) and concentrated in vacuo. The residue was dissolved in 100 mL of anhydrous ethanol containing 0.50 g 10% Pd/C and the reaction mixture was stirred under a hydrogen balloon for 24 hours. The reaction mixture was passed through a column of Celite 545 filtering agent, washed with ethanol, the filtrate was dried (MgSO₄) and concentrated in vacuo. The residue was purified by column chromatography (silica, 9.5:0.5 ,dichloromethane:methanol+1% isopropyl amine) to afford 1.65 g (52.0% yield) of the desired product.

Tert-Butyl 4-[3-(Isobutyrylamino)Phenyl]-3,6-Dihydro-1(2H)-Pyridinecarboxylate:

Into a solution of 4.00 g (16.0 mmol) of tert-butyl 4-(3-aminophenyl)-3,6-dihydro-1(2H)-pyridinecarboxylate and 5.60 mL (32.0 mmol) of diisopropylethylamine in 100 mL dichloromethane was slowly added 1.90 mL (19.0 mmol) of isobutyryl chloride. The reaction mixture was stirred at room temperature for 2 hours, washed with water, dried (MgSO₄), and concentrated in vacuo. The residue was purified by column chromatography (silica, 50:46:3:1, hexanes:dichloromethane:methanol:isopropyl amine) to afford 2.90 g (52.0% yield) of the desired product: ¹H NMR (400 MHz, CDCl₃) δ 7.69 (s, 1H), 7.34 (d, 1H, J=7.8 Hz), 7.27 (t, 1H, J=7.8 Hz), 7.11 (d, 1H, J=7.8 Hz), 6.04 (s, 1H), 4.05 (s, 2H), 3.62 (apparent t, 2H, J=4.9 Hz), 2.51 (m, 3H), 1.49 (s, 9H), 1.25 (d, 6H, J=7.4 Hz); ESMS m/e: 345.5 (M+H)⁺. Anal. Calc. for C₂₀H₂₈N₂O₃+0.175 CHCl₃: C, 66.33; H, 7.77; N, 7.67. Found: C, 66.20; H, 7.41; N, 7.88

Tert-Butyl 4-[3-(Isobutyrylamino)Phenyl]-1-Piperidinecarboxylate:

A mixture of 2.90 g (8.40 mmol) of tert-butyl 4-[3-(isobutyrylamino)phenyl]-3,6-dihydro-1(2H)-pyridinecarboxylate and 0.80 g of 10% yield Pd/C in 100 mL of ethanol was stirred under a hydrogen balloon for 24 hours. The reaction mixture was passed through a column of Celite 545 filtering agent, the filtrate was dried (MgSO₄) and concentrated in vacuo. The residue was purified by column chromatography (silica, 9.5:0.5, dichloromethane:methanol+1% isopropyl amine) to afford 2.40 g (84.0% yield) of the desired product: ¹H NMR (400 MHz, 400 MHz, CDCl₃) δ 7.49-7.44 (m, 2H), 7.24 (t, 1H, J=7.6 Hz), 6.93 (d, 1H, J=7.6 Hz), 4.20-4.10 (m, 2H), 2.86-2.45 (m, 4H), 1.86-1.75 (m, 4H), 1.48 (s, 9H), 1.24 (d, 6H, J=6.8 Hz); ESMS m/e: 345.2 (M+H)⁺; Anal. Calc. for C₂₀H₃₀N203+0.3H₂O: C, 68.27; H, 8.77; N, 7.96. Found: C, 68.25; H, 8.54; N, 7.84.

2-Methyl-N-[3-(4-Piperidinyl)Phenylipropanamide:

Into a stirred solution of 2.20 (6.50 mmol) of tert-butyl 4-[3-(isobutyrylamino)phenyl]-1-piperidinecarboxylate in 100 ml of 1,4-dioxane at 0° C. was bubbled HCl gas for 10 minutes. The reaction mixture was allowed to warm to room temperature and the bubbling of the HCl gas was continued for 1 hour. The solvent was removed in vacuo, the residue was dissolved in 50 mL of water and was neutralized by the addition of KOH pellets. The aqueous solution was extracted with 3×80 mL of dichloromethane, the combined organic extracts were dried (MgSO₄), filtered and concentrated in vacuo. The residue was purified by column chromatography (silica, 9:1, dichloromethane:methanol+1% isopropyl amine) to afford 0.700 g (46.0% yield) of the desired product: ¹H NMR (400 MHz, 400 MHz, CDCl₃) δ 7.47 (s, 1H), 7.40 (d, 1H, J=7.8 Hz), 7.24 (t, 1H, J=7.8 Hz), 7.00 (d, 1H, J=7.8 Hz), 3.23-3.14 (m, 5H), 2.82-2.57 (m, 4H), 1.20 (d, 6H, J=6.8 Hz); ESMS m/e: 247.2 (M+H)⁺; The hydrochloride salt was used for the combustion analysis: Anal. Calc. for C₁₅H₂₂N₂O+HCl+0.15 CHCl₃: C, 60.51; H, 7.76; N, 9.32. Found: C, 60.57; H, 7.83; N, 8.88.

3-(4-Piperidinyl)Aniline:

¹H NMR (400 MHz, 400 MHz, CDCl₃) δ 7.01 (t, 1H, J=7.6 Hz), 6.62-6.54 (m, 3H), 3.16 (br d, 2H, J=10.3 Hz), 2.75 (dt, 2H, J=2.7, 12.3 Hz), 2.56 (tt, 1H, J=3.6, 12.3 Hz), 1.81 (br d, 2H, J=12.3 Hz), 1.65 (dq, 2H, J=4.0, 12.3 Hz); ESMS m/e: 177.2 (M+H)⁺.

Tert-Butyl 4-(4-Nitrophenyl)-3,6-Dihydro-1(2H)-Pyridinecarboxylate:

To a 25-mL RB flask, equipped with a condenser, was added tert-butyl 4-{[(trifluoromethyl)sulfonyl]oxy}-3,6-dihydro-1(2H)-pyridinecarboxylate (1.0 g), 4-nitrophenylboronic acid (0.71 g), sodium carbonate (0.430 mL of 2M solution), lithium chloride (0.382 g), tetrakis(triphenylphosphine)- palladium (0) (0.173 g) and ethylene glycol dimethyl ether (10 mL). The reaction mixture was flushed with Argon three times, then the reaction mixture was heated to 100° C. for 3 hrs. After cooling to room temperature, the reaction mixture was diluted with methylene chloride (30 mL) and water (30 mL) and the organic layer was separated. The aqueous layer was extracted with methylene chloride (3×20 mL) and the combined organic extracts were washed with sat NH₄Cl (20 mL) and brine (20 mL), dried over MgSO₄ and concentrated under reduced pressure. The residue was purified by chromatography (6:1=hexane:ethyl acetate with 1% NH₃) to afford the product (0.55 g, 59.9%) as a yellow oil. The compound is not stable at room temperature and should be used as prompt as practical: ¹H NMR (400 MHz, 400 MHz, CDCl₃) δ 8.20 (d, 2H, J=8.6 Hz), 7.51 (d, 2H, J=8.6 Hz), 6.24 (m, 1H), 4.13 (m, 2H), 3.67 (apparent t, 2H, J=5.5 Hz), 2.55 (m, 2H), 1.49 (s, 9H).

4-(4-Nitrophenyl)-1,2,3,6-Tetrahydropyridine:

4-(4-Nitrophenyl)-1,2,3,6-tetrahydropyridine was prepared by a similar procedure to that used for the preparation of 2-methyl-N-[3-(4-piperidinyl)phenyl]propanamide using HCl gas and tert-Butyl 4-(4-Nitrophenyl)-3,6-dihydro-1(2H)-pyridinecarboxylate (130 mg) in dioxane (5.0 mL) at room temperature. The reaction mixture was concentrated in vacuo to give the crude product (69.8 mg) that used in the next reaction without further purification.

Oxazolidinone Intermediates:

Amino-(3,5-Difluorophenyl)-Acetonitrile.

Through a solution of 3,5-difluorobenzaldehyde (25.0 g, 0.176 mol) in MeOH (500 mL) in a round bottom flask, was bubbled ammonia gas for two hours at room temperature. The flask was then cooled to 0° C. and trimethylsilyl cyanide was then added slowly. The reaction mixture was stirred for 2 h, at which time TLC analysis indicated that the reaction was complete (R_{f}=0.35, 3:2 hexane/EtOAc). The solvent was removed in vacuo and the residue was subjected to flash column chromatography on silica gel to obtain the desired product.

Amino-(3,5-Difluorophenyl)-Acetic Acid Methyl Ester.

Into a well-stirred solution of amino-(3,5-difluorophenyl)-acetonitrile (22.0 g, 0.130 mol), a solution of HCl in MeOH (200 mL) was added at room temperature. The resulting yellow solution was stirred at room temperature for 10 h and was heated at reflux temperature for 1.5 h. After cooling, the solvent was removed in vacuo and the resulting yellow solid was dissolved in water (200 mL). The aqueous solution was then carefully basified with 20% NaOH solution to pH 9. The aqueous layer was extracted with CH₂Cl₂ (3×100 mL). The organic layer was separated and dried over Na₂SO₄, filtered and the solvent was removed in vacuo to obtain the desired product which was used in the next step without purification.

2-Amino-2-(3,5-Difluorophenyl)-Ethanol.

Into a well-stirred suspension of LiAlH₄ (4.7 g, 0.125 mol) in THF (120 mL) in a 3-necked round bottom flask fitted with a condenser and a dropping funnel, was added a solution of amino-(3,5-difluorophenyl)-acetic acid methyl ester (10.0 g, 0.05 mol) in THF (100 mL) dropwise at 0° C. The resulting greenish brown suspension was heated at reflux temperature for 2 h. The reaction mixture was cooled to 0° C. and then carefully quenched sequentially with 5 mL of water, 5 mL of 3N NaOH followed by 15 mL of water. The resulting suspension was filtered through a fritted glass funnel. To the filter cake was added 100 mL Et₂O and the suspension was heated at reflux temperature for 20 min. The suspension was filtered and the combined filtrates were dried over MgSO₄, filtered and the solvent was removed in vacuo. 2-Amino-2-(3,5-difluorophenyl)-ethanol was obtained as a yellow glassy syrup which was used in the next step without further purification.

[1-(3,4-Difluorophenyl)-2-Hydroxy-Ethyl]-Carbamic Acid-Tert-Butyl Ester.

Into a solution of 2-amino-2-(3,4-difluorophenyl)-ethanol (8.6 g, 49.7 mmol) in CHCl₃ (150 mL) at 0° C. was added a solution of di-tert-butyl dicarbonate (11.4 g, 52.0 mmol) in CHCl₃ (50 mL) in one portion and the resulting solution was stirred overnight at room temperature. The solvent was removed in vacuo and the residue was subjected to column chromatography on silica gel (2:1 hexane-EtOAc followed by EtOAc) to obtain (1-(3,4-difluorophenyl)-2-hydroxy-ethyl]-carbamic acid-tert-butyl ester as a white solid (10.0 g, 74% yield).

(+)-4-(3,4-Difluorophenyl)-Oxazolidin-2-One.

Into a well-stirred suspension of NaH (1.1 g, 45.8 mmol) in THF (40 mL) at R.T. was added a solution of [1-(3,4-difluorophenyl)-2-hydroxy-ethyl]-carbamic acid-tert-butyl ester (5.0 g, 18.3 mmol) in THF (20 mL) via a dropping funnel at room temperature. The resulting suspension was stirred for 3 h and then quenched carefully with 10 mL of water. The biphasic mixture was extracted with 100 mL of Et₂O, washed with brine, filtered and the solvent was removed in vacuo. The gummy residue thus obtained was purified by column chromatography over silica gel (R_{f}=0.15, 3:2 hexane-EtOAc) to obtain 4-(3,5-difluorophenyl)-oxazolidin-2-one as a white flaky solid (2.8 g, 77% yield). M.P. 81-83° C.; ¹H NMR (300 MHz, CDCl₃) δ 4.13 (dd, J=6.6 Hz, J=8.7 Hz, 1H), 4.73 (t, J=8.7 Hz, 1H), 4.94 (dd, J=6.6 Hz, J=8.7 Hz, 1H), 6.08 (br s, 1H), 7.03-7.23 (m, 3H). The enantiomers were separated on a Chiralcel OD (20×250 mm) using 80% hexane/20% isopropyl alcohol as the eluting system at 12.0 mL/min (U.V. 254 nm). The retention times for the two isomers were 16.19 min and 20.08 min respectively.

4-Nitrophenyl (4S)-4-(3,4-Difluorophenyl)-2-oxo-1,3-Oxazolidine-3-Carboxylate:

Into a suspension of NaH (0.14 g, 5.30 mmol) in 20 mL of anhydrous THF under argon, a solution of (+)-4-(3,5-difluorophenyl)-oxazolidin-2-one (0.88 g, 4.42 mmol) in THF was added dropwise (dropping funnel). The resulting suspension was stirred at room temperature for 30 min. This suspension was then added dropwise via cannula into another round bottom flask containing a solution of 4-nitrophenylchloroformate (1.11 g, 5.30 mmol) in 25 mL of THF and cooled at −78° C. over a period of 15 min. The stirring was continued for 2 h after which the solvent was removed and the residue was purified by column chromatography on silica gel with 1:1 hexane/CH₂Cl₂ followed by CH₂Cl₂ (R_{f}=0.4, CH₂Cl₂) to obtain the desired product as a white solid (1.55 g, 86% yield).

Similarly, following the above procedure, 4-(3,5-trifluorophenyl)-2-oxo-oxazolidine-3-carboxylic acid-4-nitro-phenyl ester and 4-(3,4,5-trifluorophenyl)-2-oxo-oxazolidine-3-carboxylic acid-4-nitro-phenyl ester were obtained. The oxazolidinone enantiomers were resolved on a chiracel OD column (as in the previous example) and the 4-nitro-phenyl esters were prepared using 4-nitrophenyl chloroformate.

4-Nitrophenyl (4S)-4-(3,5-Difluorophenyl)-2-oxo-1,3-Oxazolidine-3-Carboxylate:

¹H NMR (400 MHz, CDCl₃) δ 8.26 (d, 2H, J=9.3 Hz), 7.33-6.81 (m, 5H), 5.41 (dd, 1H, J=4.1, 8.7 Hz), 4.81 (t, 1H, J=9.3 Hz), 4.33 (dd, 1H, J=4.1, 9.3 Hz); Anal. Calc. for C₁₆H₁₀F₂N₂O₆+0.2EtOAc: C, 52.84; H, 3.06; N, 7.34. Found: C, 53.26; H, 2.83; N, 7.73

4-Nitrophenyl (4S)-2-oxo-4-(3,4,5-Trifluorophenyl)-1,3-Oxazolidine-3-Carboxylate:

¹H NMR (400 MHz, CDCl₃) δ 8.27 (d, 2H, J=9.0 Hz), 7.31 (d, 2H, J=9.0 Hz), 7.11-7.02 (m, 2H), 5.37 (dd, 1H, J=4.1, 9.0 Hz), 4.81 (apparent t, 1H, J=9.0 Hz), 4.33 (dd, 1H, J=4.1, 9.0 Hz); Anal. Calc. for C₁₆H₉F₃N₂O₆: C, 50.27; H, 2.37; N, 7.33. Found: C, 50.56; H, 2.50; N, 7.49.

1-(3,4-Difluorophenyl)-2-Methyl-2-Hydroxypropylamine.

Into a well-stirred solution of methyl 2-amino-2-(3,4-difluorophenyl)acetate (10.5 g, 52.19 mmol) in anhydrous ether (200 mL) at 0° C. a solution of methylmagnesium bromide (3 M, 87 mL, 261 mmol) in ether was added over 10 minutes. The reaction mixture was stirred at 0° C. for 2.5 h and allowed to warm to room temperature. After 12 h, the reaction mixture was carefully poured onto a mixture of ice (300 g) and saturated aqueous ammonium chloride (50 g). The ether layer was separated and the aqueous layer was extracted with more ether (4×200 mL). The combined extracts were dried with magnesium sulfate and the solvent evaporated. The crude product was purified by column chromatography on silica gel using chloroform/methanol/2M ammonia in methanol (1000:20:10, 1000:40:20, 1000:80:40) as the eluent to give the product as an oil (6.5 g, 62% yield). The ¹H-NMR and MS confirmed this to be the desired product.

4-(3,4-Difluorophenyl)-5,5-Dimethyl-2-oxo-Oxazolidine.

A mixture of 1-(3,4-difluorophenyl)-2-methyl-2-hydroxypropylamine (3.00 g, 14.9 mmol) and carbonyldiimidazole (2.418 g, 14.9 mmol) in dichloromethane (150 mL) was heated at reflux temperature for 36 h and the solvent evaporated. The residue was purified by column chromatography on silica gel using chloroform/ethyl acetate (9:1) to give the product as a viscous oil which solidified on standing (1.80 g, 50% yield).

4-(3,4-Difluorophenyl)-5,5-Dimethyl-2-oxo-3-(4-Nitrophenyloxycarbonyl)Oxazolidine.

Into a stirred suspension of sodium hydride (60% suspension in paraffin 203 mg, 1.4 eq.) in THF (20 mL) at 0° C., a solution of 4-(3,4-difluorophenyl)-5,5-dimethyl-2-oxo-oxazolidine (870 mg, 3.622 mmol) in THF (5 mL) was added followed by stirring for 30 minutes. This suspension was added to a solution of 4-nitrophenyl chloroformate (950 mg, 4.71 mmol) in THF (20 mL) at −78° C. under argon and the stirring was continued for 2 h. It was slowly warmed to room temperature and after 4 h the solvent was evaporated. The residue was mixed with dichloromethane (150 mL), washed with 0.05 N sodium hydroxide (3×10 mL), and dried (sodium sulfate). The solvent was evaporated and the residue was purified by column chromatography on silica gel using chloroform/ethyl acetate (9:1) as the eluent to give the product as a white powder (860 mg, 59% yield).

4-Nitrophenyl 4-(3,4-Difluorophenyl)-5,5-Dimethyl-2-oxo-1,3-Oxazolidine-3-Carboxylate:

¹H NMR (400 MHz, CDCl₃) δ 8.24 (d, 2H, J=9 Hz), 7.29-6.97 (m, 5H), 5.04 (s, 1H), 1.09 (s, 6H); Anal. Calc. for C₁₈H₁₄F₂N₂O₆+0.2% H₂O: C, 54.61; H, 3.67; N, 7.08. Found: C, 54.89; H, 3.59; N, 7.41.

a. Benzhydrylindene-(3,4-Difluoro-Benzyl)-Amine

Into a solution of 3,4-difluorobenzylamine (9.8 g, 69 mmol) and benzophenone (13.0 g, 71.0 mmol) in toluene (200 mL) was added a catalytic amount of BF₃.OEt₂ and the resulting solution was heated at reflux temperature for 12 h. The reaction mixture was concentrated in vacuo, yielding an oil (21 g, >95%), which was characterized by NMR analysis and subjected to the following reaction without any further purification. ¹H NMR (CDCl₃) δ 4.57 (s, 2H), 7.80-6.80 (m, 13H).

b. 1-(Benzhydryliden-Amino)-1-(3,4-Difluoro-Phenyl)-Propan-2-ol.

Into a solution of the benzhydrylindene-(3,4-difluoro-benzyl)-amine (21 g, 69 mmol) in 250 ml of dry THF was added tert-butyllithium (1.7 M, 60 ml) dropwise and the resulting solution was stirred at −78° C. for 0.5 h. To the solution was added acetaldehyde (10 ml, 180 mmol) in 100 ml of THF and the solution was stirred at −78° C. for 2 h and 25° C. for 1 h. The reaction mixture was quenched by addition of brine. The reaction mixture was diluted with 500 ml of Et₂O and washed with brine. The organic layer was dried over Na₂SO₄ and concentrated in vacuo to give an oil, which was taken to the next step without any further purification. ¹H NMR (CDCl3) δ 1.04 (d, 3H), 2.77 (broad s. 1H), 4.08(m, 1H), 4.15 (d, 1H), 7.80-6.80 (m, 13H).

c. 1-Amino-1-(3,4-Difluoro-Phenyl)-Propan-2-ol

A solution of crude product from the previous procedure and MeONH₂.HCl (10 g, 120 mmol) was diluted in 200 ml of MeOH and stirred for 12 h. The reaction mixture was concentrated in vacuo, yielding an oily residue, which was re-dissolved in 200 ml of EtOAc and washed with brine. The organic layer was concentrated in vacuo to produce an oily mixture, which was subjected to column chromatography (5% NH₃ saturated MeOH/CHCl₃) to yield the desired product (8.8 g, 68% yield from 3,4-difluorobenzylamine) as a mixture of diastereomers. ¹H NMR (CDCl₃) (˜4:1 mixture of the diastereomers) δ 1.02 (d, J=6.0 Hz, 3H), 1.04 (d, J=6.3 Hz, 3H), 2.10 (br, 6H), 3.56-3.69 (m, 2H), 3.88-3.92 (m, 2H), 7.02-7.17 (m, 6H).

d. [1-(3,4-Difluorophenyl)-2-Hydroxy-Propyl]-Carbamic Acid-Tert-Butyl Ester

Into a solution of 1-amino-1-(3,4-difluorophenyl)-propan-2-ol (13.1 g, 70.1 mmol) in CHCl₃ (150 mL) at 0° C. was added a solution of di-tert-butyl dicarbonate (19.3 g, 87.6 mmol) in CHCl₃ (50 mL) in one portion and the resulting solution was stirred overnight at room temperature. The solvent was removed in vacuo and the residue was subjected to column chromatography on silica gel (2:1 hexane-EtOAc followed by EtOAc) to obtain [1-(3,4-difluorophenyl)-2-hydroxy-propyl]-carbamic acid-tert-butyl ester as a viscous oil (18.4 g, 91% yield). ¹H NMR (CDCl₃) (4:1 mixture of the diastereomers) δ 1.05 (d, J=6.6 Hz, 3H), 1.25 (d, J=6.0 Hz, 3H), 1.41 (br, 20H), 3.92-4.19 (br, 2H), 4.45-4.60 (m, 2H), 5.41-5.49 (br, 2H), 7.02-7.17 (m, 6H).

e. 4-(3,4-Difluorophenyl)-5-Methyl-Oxazolidin-2-One

Into a well-stirred solution of [1-(3,4-difluorophenyl)-2-hydroxy-propyl]-carbamic acid-tert-butyl ester (0.43 g, 1.5 mmol) THF (20 mL) was added 95% NaH (0.09 g, 3.8 mmol) at room temperature. When the reaction was carried out on a larger (>5 g) scale, 1.0 equivalent of KH and 1.5 eq. of NaH was used as the base. The resulting suspension was stirred for 3 h at about 35° C. (warm water bath) and then quenched carefully with ice. The biphasic mixture was extracted with 100 mL of EtOAc, washed with brine, dried over Na₂SO₄, filtered and the solvent was removed in vacuo. The two diastereomers were separated by column chromatography over silica gel (First isomer: 0.16 g, R_{f}=0.6, 3:1 hexane-EtOAc; second isomer: 0.18 g, R_{f}=0.5, 3:1 hexane-EtOAc). NOE experiment suggested that the first diastereomer had the methyl and the aryl group in trans configuration while the second diastereomer had cis relationship between the two groups.

The ¹H NMR spectrum for the trans diastereomers is as follows. ¹H NMR (CDCl₃) δ 1.49 (d, J=6.0 Hz, 3H), 4.37 (dq, J=6.0 Hz, J=7.2 Hz, 1H), 4.45 (d, J=7.2 Hz, 1H), 6.63 (br s, 1H), 7.08-7.28 (m, 3H).

The ¹H NMR spectrum for the cis diastereomers is as follows. ¹H NMR (CDCl₃) δ 0.96 (d, J=6.6 Hz, 3H), 4.91 (d, J=8.1 Hz, 1H), 4.99 (dq, J=6.6 Hz, J=8.1 Hz, 1H), 6.63 (br s, 1H), 7.08-7.28 (m, 3H).

Enantiomers of the diastereomers were separated by HPLC by using a Chiralcel OD column (20×250 mm) with 80% hexane/20% isopropyl alcohol/ 0.1% diethylamine as the eluting system (12 mL/min) under isocratic conditions (U.V. 254 nM).

f. 4-(3,4-Difluorophenyl)-5-Methyl-2-oxo-Oxazolidine-3-Carboxylic Acid-4-Nitro-Phenyl Ester

Into a solution of 4-(3,4-difluorophenyl)-5-methyl-oxazolidin-2-one (0.97 g, 4.55 mmol) in 60 mL THF was added a solution of n-butyllithium in hexane (3.06 mmol, 4.9 mmol) dropwise via a syringe under argon atmosphere at −78° C. The resulting yellow solution was stirred at −78° C. for 40 min. This solution was then added dropwise via a cannula into another round bottom flask containing a solution of 4-nitrophenylchloroformate (1.03 g, 5.1 mmol) in 60 mL of THF, cooled at −78° C., over a period of 15 min. After five minutes, the flask was removed from the cooling bath and stirring was continued for 1 h. The reaction mixture was quenched by adding ice and it was extracted with EtOAc. The organic extracts were washed with brine and the organic layer was dried over Na₂SO₄. The solvent was removed after filtration and the residue was purified by column chromatography on silica gel with 1:1 hexane/CH₂Cl₂ followed by CH₂Cl₂ (Rf=0.4, CH₂Cl₂) to give the desired product.

The relative configurations of the cis and trans isomers were assigned on the basis of ¹H NMR analysis of the respective p-nitrophenyloxycarbonyl derivatives. For the trans isomer, an NOE was observed between the protons of the C-5 methyl group and the proton at C-4. No NOE was observed between the protons at the C-4 and C-5 positions of this isomer, which was thus assigned trans stereochemistry. For the cis isomer, no NOE was observed between the protons of the C-5 methyl group and the proton at C-4. However, a NOE was observed between the protons at the C-4 and C-5 positions, leading us to assign this isomer cis stereochemistry. The vicinal coupling constants of the C-4 protons of cis (J=7.8 Hz) and trans (J=5.1 Hz) are also consistent with the values reported for similar oxazolidinones, and were thus helpful in making the stereochemical assignments (Dondoni, A.; Perrone, D.; Semola, T. Synthesis 1995, 181).

In order to assign the absolute configurations at the stereogenic centers of the oxazolidinone rings, a new synthetic route was designed which employed an enantiomerically pure substrate derived from the chiral pool. Commercially available (S)-(+)-methyl lactate was converted into its pyrrolidine amide according to the method of Martin et al (Martin, R.; Pascual, O.; Romea, P.; Rovira, R.; Urpi, F.; Vilarrasa, J. Tetrahedron Lett. 1997, 38, 1633). Following the protection of the hydroxy group of (2S)-1-oxo-1-(1-pyrrolidinyl)-2-propanol to a TBDMS group, treatment of tert-butyl(dimethyl)silyl (1S)-1-methyl-2-oxo-2-(1-pyrrolidinyl)ethyl ether with 3,4-difluorophenyllithium yielded (2S)-2-{[tert-butyl(dimethyl)silyl]oxy}-1-(3,4-difluorophenyl)-1-propanone as the sole product, which was then converted to (2S)-2-{[tert-butyl(dimethyl)silyl]oxy}-1-(3,4-difluorophenyl)-1-propanone oxime. Reduction of the (2S)-2-{[tert-butyl(dimethyl)silyl]oxy}-1-(3,4-difluorophenyl)-1-propanone oxime with LiAlH₄, N-acylation, and base induced cyclization provided oxazolidinone diastereomers, which were separated by flash column chromatography. The enantiomeric purity of these isomers was confirmed by chiral HPLC analysis and their relative configurations were assigned by comparison of their ¹H NMR spectra with those of the racemic isomers. As the absolute configuration at C-5 of the lactic acid derived oxazolidinone described above is (S), the C-4 center in trans compounds also has the (S) configuration. Accordingly, the absolute configurations for the stereogenic centers in the cis compounds are assigned accordingly (4R,5S).

4-Nitrophenyl (4S,5R)-4-(3,4-Difluorophenyl)-5-Methyl-2-oxo-1,3-Oxazolidine-3-Carboxylate:

¹H NMR (400 MHz, CDCl₃) δ 8.25 (d, 2H, J=8.8 Hz), 7.30-6.99 (m, 5H), 5.35 (d, 1H, J=7.7 Hz), 5.07 (apparent quintet, 1H), 1.17 (d, 3H, J=6.5 Hz); Anal. Calc. for C₁₇H₁₂F₂N₂O₆+0.5H₂O: C, 52.72; H, 3.38; N, 7.23. Found: C, 53.09; H, 3.19; N, 7.50.

(+)-2-Amino-3-(3,4-Difluoro)-Phenyl-Propan-1-ol:

(+)-3,4-difluorophenyl alanine (1.0 g, 5.0 mmol) was added in small portions to a stirring suspension of LiAlH₄ (0.480 g, 12.5 mmol) in THF (30 mL) at 0° C. The resulting gray suspension was then heated at reflux for 2 h. The reaction mixture was cooled to 0° C. and then carefully quenched sequentially with water (0.5 mL), 3 N NaOH (0.5 mL), and water (1.50 mL). The resulting suspension was filtered through a fritted glass funnel. Ether (50 mL) was added to the filter cake and the suspension was heated at reflux temperature for 20 min. The suspension was filtered and was combined with the previous filtrate. The combined organics were dried over MgSO₄, filtered and the solvent was removed in vacuo. 2-Amino-3-(3,4-difluoro)-phenyl-propan-1-ol was obtained as a white solid (0.500 g, 100%) which was used in the next step without further purification.

(+)-[1-(3,4-Difluorobenzyl)-2-Hydroxy-Ethyl]-Carbamic Acid-Tert-Butyl Ester:

A solution of di-tert-butyl dicarbonate (0.640 g, 2.90 mmol) in CHCl₃ (10 mL) was added in one portion to a solution of (+)-2-amino-3-(3,4-difluoro)-phenyl-propan-1-ol (0.500 g, 2.62 mmol) in CHCl₃ (20 mL) at 0° C. and the resulting solution was stirred overnight at room temperature. The solvent was removed in vacuo and the residue was chromatographed (2:1 hexane-EtOAc, followed by EtOAc), giving (+)-[1-(3,4-difluorobenzyl)-2-hydroxy-ethyl]-carbamic acid-tert-butyl ester as a white solid (0.640 g, 99%).

(+)-4-(3,4-Difluoro-Benzyl)-Oxazolidin-2-ONE:

A solution of (+)-[1-(3,4-difluorobenzyl)-2-hydroxy-ethyl]-carbamic acid-tert-butyl ester (1.00 g, 4.00 mmol) in THF (10 mL) was added via a dropping funnel to a stirring suspension of 95% NaH (0.12 g, 5.0 mmol) in THF (20 mL) at room temperature. The resulting suspension was stirred for 3 h and then quenched carefully with water (10 mL). The biphasic mixture was extracted with Et₂O (50 mL), washed with brine, filtered and the solvent was removed in vacuo. The resulting gummy residue was purified by column chromatography (R_{f}=0.25, 3:2 hexane-EtOAc), to give the desired product as a white solid (0.320 g, 76%).

(+)-4-(3,4-Difluoro-Benzyl)-Oxazolidin-2-one-3-Carboxylic Acid-4-Nitro-Phenyl Ester:

A solution of (+)-4-(3,4-difluoro-benzyl)-oxazolidin-2-one (0.210 g, 1.0 mmol) in THF (10 mL) was added dropwise via a dropping funnel to a stirring suspension of NaH (30.0 mg, 1.30 mmol) in anhydrous THF (10 mL) under argon. The resulting suspension was stirred at room temperature for 30 min. This suspension was then added dropwise via cannula to a solution of 4-nitrophenylchloroformate (0.300 g, 1.50 mmol) in THF (20 mL) at −78° C. over 15 min. Stirring was continued for 2 h after which the solvent was removed and the residue was purified by column chromatography (1:1 hexane/CH₂Cl2, followed by CH₂Cl₂; Rf=0.4, CH₂Cl₂), to give the desired product as a yellow solid (0.350 g, 82%).

Similarly, 4-nitrophenyl 4-(4-fluorobenzyl)-2-oxo-1,3-oxazolidine-3-carboxylate was obtained from the corresponding p-fluorophenyl alanine:

4-Nitrophenyl 4-(4-Fluorobenzyl)-2-oxo-1,3-Oxazolidine-3-Carboxylate:

¹H NMR (400 MHz, CDCl₃) δ 8.32 (d, 2H, J=9.3 Hz), 7.42 (d, 2H, J=8.9 Hz), 7.24-6.99 (m, 4H), 4.69-4.59 (m, 1H), 4.35 (t, 1H, J=8.6 Hz), 4.23 (dd, 1H, J=2.7, 9.3 Hz), 3.37 (dd, 1H, J=3.8, 13.6 Hz), 2.94 (dd, 1H, J=9.3, 13.6 Hz); Anal. Calc. for C₁₇H₁₃FN₂O₆: C, 56.67; H, 3.64; N, 7.77. Found: C, 56.94; H, 3.76; N, 7.71.

2-[6-(4-Phenyl-1-Piperidinyl)Hexyl]-1H-Isoindole-1,3(2H)-Dione:

To the 500 ml RB-flask was added 4-phenylpiperidine hydrochloride (5 g, 25 mmol), N-(6-bromohexyl)phthalimide (15.5 g, 50 mmol), N,N-diisopropylethylamine (21.8 ml, 125 mmol), tetrabutylammonium iodide (0.2 g), and dioxane (250 ml) at room temperature. The reaction mixture was stirred at 100° C. for 72 h. The solvent was removed in vacuo and the crude product was purified by flash chromatography (98:2=Chloroform:2N ammonia in methanol) to afford 7.67 g of the desired product (77% yield): ¹H NMR (400 MHz, CDCl₃) δ 7.78-7.79 (m, 2H), 7.74-7.65 (m, 2H), 7.32-7.14 (m, 5H), 3.69 (t, 2H, J=7.35 Hz), 3.06 (d, 2H, J=11.0 Hz), 2.49 (quintet, 1H, J=7.6 Hz), 2.36 (t, 2H, J=7.6 Hz), 2.02 (t, 2H, J=12.5 Hz), 1.82 (br s, 4H), 1.69 (t, 2H, J=6.3 Hz), 1.54 (br s, 2H), 1.37 (br s, 4H); ESMS m/e: 391.3 (M+H)⁺; Anal. Calc. for C₂₅H₃₀N₂O₂+0.2H₂O: C, 76.19; H, 7.77; N, 7.11. Found: C, 76.14; H, 7.38; N, 7.13.

General Procedure for the Preparation of the Substituted 4-[4-(3-aminophenyl)-1-piperidinyl]-1-(phenyl)-1-butanones:

A mixture of 4-(3-aminophenyl)piperidine (2.0 mmol), 2.4 mmol of the appropriate substituted phenyl butyryl chloride, 3.0 mmol of K₂CO₃, and 10 mg of 18-crown-6 in 5 mL of toluene were heated at 110° C. for 2.5 days. The reaction mixture was concentrated and chromatographed on silica (5% methanol in dichloromethane) to give the desired compound:

4-[4-(3-Aminophenyl)-1-Piperidinyl]-1-(4-Phenoxyphenyl)-1-Butanone:

305 mg; ESMS m/e: 415.4 (M+H)⁺.

4-[4-(3-Aminophenyl)-1-Piperidinyl]-1-(4-Chlorophenyl)-1-Butanone:

500 mg; Anal. Calc for C₂₁H₂₅ClN₂O+0.3H₂O: C, 69.62; H, 7.12; N, 7.73. Found: C, 69.63; H, 7.34; N, 7.60; ESMS m/e: 357.3 (M+H)⁺.

4-[4-(3-Aminophenyl)-1-Piperidinyl]-1-Phenyl-1-Butanone:

250 mg; Anal. Calc for C₂₁H₂₆N₂O+0.2H₂O: C, 77.36; H, 8.16; N, 8.59. Found: C, 77.55; H, 8.12; N, 8.75; ESMS m/e: 323.3 (M+H)⁺

4-[4-(3-Aminophenyl)-1-Piperidinyl]-1-(2,4-Dimethoxyphenyl)-1-Butanone:

330 mg; Anal. Calc for C₂₃H₃₀N₂O₃+0.5H₂O: C, 70.56; H, 7.98; N, 7.16. Found: C, 70.69; H, 7.87; N, 6.99; ESMS m/e: 383.3 (M+H)⁺

General Procedure for the Acylation or Sulfonylation of the Substituted 4-[4-(3-Aminophenyl)-1-piperidinyl]-1-(4-phenyl)-1-butanones:

A mixture of 1 equivalent of a substituted 4-[4-(3-aminophenyl)-1-piperidinyl]-1-(4-phenyl)-1-butanone, 1.5 equivalent of an acid chloride or a sulfonyl chloride, and 5 equivalents of diisopropylethylamine, in dichloromethane was stirred at room temperature for two days. The reaction mixture was applied to a preparative TLC plate and eluted with dichloromethane: methanol (15:1, containing 1% isopropyl amine) to give the desired product.

General Procedure for the Preparation of the Substituted 4-N-(3-{1-[4-(phenyl)-4-oxobutyl]-4-piperidinyl}phenyl)acetamides:

A mixture of N-[3-(4-piperidinyl)phenyl]acetamide (1.0 eq) and an aryl substituted chlorobutyrophenone (2.0 eq), K₂CO₃ (5.0 eq), diisopropylethylamine (3.0 eq) and tetrabutylammonium iodide (cat. 5-10%) in dioxane (0.5 to 1.0 M) were heated at reflux temperature for 16 h. The reaction mixture was filtered and concentrated in vacuo. The crude product was chromatographed using silica preparative TLC (chloroform:methanol containing 0.5% isopropyl amine) to give the desired product.

### EXAMPLE 57

N-(3-{1-[4-(3,4-Dimethylphenyl)-4-Oxobutyl]-4-Piperidinyl)Phenyl)Acetamide:

¹H NMR (CDCl₃) δ 7.75 (s, 1H), 7.71 (d, 1H, J=7.6 Hz), 7.45 (d, 2H, J=7.2 Hz), 7.35 (s, 1H), 7.26-7.22 (m, 2H), 6.93 (d, 1H, J=7.6 Hz), 3.24-3.21 (m, 2H), 3.04 (t, 2H, J=7.0 Hz), 2.67-2.63 (m, 2H), 2.59-2.48 (m, 1H), 2.32 (s, 6H), 2.30-2.27 (m, 2H), 2.18 (s, 3H), 2.14-2.06 (m, 2H), 2.00-1.80 (m, 4H); ESMS m/e: 393.3 (M+H)⁺.

### EXAMPLE 58

N-(3-{1-[4-(3,4-Dimethylphenyl)-4-Oxobutyl]-4-Piperidinyl}Phenyl)-2-Methylpropanamide:

A mixture of 0.0500 g (0.200 mmol) of 2-methyl-N-[3-(4-piperidinyl)phenyl]propanamide, 0.100 g (0.480 mmol) of 4-chloro-3′,4′-dimethylbutyrophenone, 0.080 g (0.600 mmol) of K₂CO₃ and 0.090 g (0.600 mmol) of NaI in 5 mL of DMF was heated at reflux temperature for 18 hours. The reaction mixture was filtered, the filtrate was poured into 5 mL of water and washed with 3×5 mL of ethyl acetate. The combined organic extracts were dried (MgSO₄), concentrated in vacuo and purified by preparative TLC (silica; 9.5:0.5, dichloromethane:methanol+1% isopropyl amine) to afford 0.067 g (80.0% yield) of the desired product:¹H NMR (400 MHz, CDCl₃) δ 7.72 (d, 1H, J=8.0 Hz), 7.44 (s, 1H), 7.38 (d, 1H, J=8.0 Hz), 7.23-7.20 (m, 2H), 7.16 (s, 1H), 6.95 (d, 1H, J=6.8 Hz), 3.13-3.11 (m, 2H), 3.02 (t, 2H, J=7.0 Hz), 2.56-2.40 (m, 4H), 2.32 (s, 6H), 2.17-2.15 (m, 2H), 2.04-1.78 (m, 6H), 1.25 (d, 6H, J=6.8 Hz); ESMS m/e: 421.3 (M+H)⁺.

### EXAMPLE 59

N-(3-{1-[4-(3,4-Dimethylphenyl)-4-Oxobutyl]-4-Piperidinyl}Phenyl)Cyclohexanecarboxamide:

¹H NMR (400 MHz, CDCl₃) δ 7.80-6.81 (m, 7H), 3.41-3.00 (m, 4H), 2.95-2.41 (m, 4H), 2.32 (s, 6H), 2.22-1.05 (m, 18H); ESMS m/e 461.4 (M+H)⁺.

### EXAMPLE 60

N-(3-{1-[4-(3,4-Dimethylphenyl)-4-Oxobutyl]-4-Piperidinyl}Phenyl)-2-Phenylacetamide:

¹H NMR (400 MHz, CDCl₃) δ 7.85-7.65 (m, 2H), 7.45-6.92 (m, 10H), 3.76 (s, 2H), 3.10-2.90 (m, 4H), 2.50-2.35 (m, 3H), 2.32 (s, 6H), 2.10-1.85 (m, 4H), 1.80-1.60 (m, 4H); ESMS m/e: 469.4 (M+H)⁺.

### EXAMPLE 61

N-(3-(1-[4-(3,4-Dimethylphenyl)-4-Oxobutyl]-4-Piperidinyl}Phenyl)-2-(3-Methoxyphenyl)Acetamide:

¹H NMR (400 MHz, CDCl₃) δ 7.76-7.65 (m, 2H), 7.38-7.12 (m, 6H), 6.95-6.80 (m, 3H), 3.82 (s, 3H), 3.70 (s, 2H), 3.10-2.90 (m, 4H), 2.50-2.38 (m, 3H), 2.32 (s, 6H), 2.10-1.85 (m, 4H), 1.80-1.60 (m, 4H); ESMS m/e: 499.4 (M+H)⁺.

### EXAMPLE 62

N-(3-{1-[4-(3,4-Dimethylphenyl)-4-Oxobutyl]-4-Piperidinyl}Phenyl)-2-Methoxyacetamide:

¹H NMR (400 MHz, CDCl₃) δ 7.80-7.75 (m, 2H), 7.50-7.38 (m, 2H), 7.34-6.90 (m, 3H), 4.00 (s, 2H), 3.51 (s, 3H), 3.30-2.95 (m, 4H), 2.70-2.50 (m, 3H), 2.32 (s, 6H), 2.15-1.80 (m, 8H); ESMS m/e: 423.3 (M+H)⁺.

### EXAMPLE 63

N-(3-{1-[4-(3,4-Dimethylphenyl)-4-Oxobutyl]-4-Piperidinyl}Phenyl)Methanesulfonamide:

¹H NMR (400 MHz, CDCl₃) δ 7.82-7.10 (m, 7H), 3.41 (s, 3H), 3.40-2.85 (m, 4H), 2.82-2.35 (m, 5H), 2.32 (s, 6H), 2.22-1.80 (m, 6H); ESMS m/e: 429.3 (M+H)⁺.

### EXAMPLE 64

N-(3-{1-[4-(3,4-Dimethylphenyl)-4-Oxobutyl]-4-Piperidinyl}Phenyl)Ethanesulfonamide:

¹H NMR (400 MHz, CDCl₃) δ 7.75 (s, 1H), 7.71 (d, 1H, J=7.6 Hz), 7.30-7.09 (m, 4H), 7.02 (d, 1H, J=7.2 Hz), 3.36-3.05 (m, 6H), 2.77-2.52 (m, 3H), 2.32 (s, 6H), 2.15-1.82 (m, 8H), 1.37 (t, 3H, J=7.4 Hz); ESMS m/e: 443.3 (M+H)⁺

### EXAMPLE 65

N-(3-{1-[4-(4-Chlorophenyl)-4-Oxobutyl]-4-Piperidinyl}Phenyl)Acetamide:

¹H NMR (400 MHz, CDCl₃) δ 7.92 (d, 2H, J=8.8 Hz), 7.55-7.40 (m, 3H), 7.35 (s, 1H), 7.22 (t, 1H, J=8.0 Hz), 6.92 (d, 1H, J=8.0 Hz), 3.30-3.27 (m, 2H), 3.09 (t, 2H, J=7.0 Hz), 2.76-2.39 (m, 5H), 2.20 (s, 3H), 2.17-1.85 (m, 6H); ESMS m/e 399.3 (M+H)⁺.

### EXAMPLE 66

N-(3-{1-[4-(4-Chlorophenyl)-4-Oxobutyl]-4-Piperidinyl}Phenyl)-2-Methylpropanamide:

¹H NMR (400 MHz, CDCl₃) δ 7.93 (d, 2H, J=8.6 Hz), 7.45 (d, 2H, J=8.6 Hz), 7.39 (d, 1H, J=7.2 Hz), 7.32 (s, 1H), 7.24 (t, 1H, J=7.8 Hz), 6.94 (d, 1H, J=8.4 Hz), 3.21-3.18 (m, 2H), 3.05 (t, 2H, J=7.0 Hz), 2.64-2.51 (m, 4H), 2.28-1.86 (m, 8H), 1.26 (d, 6H, J=6.8 Hz); ESMS m/e: 427.3 (M+H)⁺.

### EXAMPLE 67

N-(3-{1-[4-(4-Chlorophenyl)-4-Oxobutyl]-4-Piperidinyl}Phenyl)Cyclohexanecarboxamide:

¹H NMR (400 MHz, CDCl₃) δ 7.93 (d, 2H, J=8.4 Hz), 7.55-7.19 (m, 5H), 6.93 (d, 1H, J=7.6 Hz), 3.25-3.00 (m, 4H), 2.65-2.45 (m, 4H), 2.30-1.50 (m, 18H); ESMS m/e: 467.3 (M+H)⁺.

### EXAMPLE 68

N-(3-{1-[4-(4-Chlorophenyl)-4-Oxobutyl]-4-Piperidinyl}Phenyl)-2-Phenylacetamide:

¹H NMR (400 MHz, CDCl₃) δ 7.92 (d, 2H, J=8.4 Hz), 7.46-7.26 (m, 9H), 7.20 (t, 1H, J=7.6 Hz), 6.92 (d, 1H, J=7.6 Hz), 3.75 (s, 2H), 3.15-3.13 (m, 2H), 3.03 (t, 2H, J=7.0 Hz), 2.64-2.46 (m, 3H), 2.22-1.60 (m, 8H); ESMS m/e: 475.3 (M+H)⁺.

### EXAMPLE 69

N-(3-{1-[4-(4-Chlorophenyl)-4-Oxobutyl]-4-Piperidinyl}Phenyl)-2-(3-Methoxyphenyl)Acetamide:

¹H NMR (400 MHz, CDCl₃) δ 7.92 (d, 2H, J=8.4 Hz), 7.44 (d, 2H, J=8.4 Hz) 7.38 (s, 1H), 7.35-7.25 (m, 3H), 7.19 (t, 1H, J=7.8 Hz), 6.94-6.86 (m, 3H), 3.81 (s, 3H), 3.72 (s, 2H), 3.12-3.09 (m, 2H), 3.02 (t, 2H, J=6.8 Hz), 2.57-2.44 (m, 3H), 2.20-1.60 (m, 8H); ESMS m/e: 505.3 (M+H)⁺.

### EXAMPLE 70

N-(3-{1-[4-(4-Chlorophenyl)-4-Oxobutyl]-4-Piperidinyl}Phenyl)-2-Methoxyacetamide:

¹H NMR (400 MHz, CDCl₃) δ 7.93 (d, 2H, J=8.4 Hz), 7.50-7.25 (m, 5H), 6.98 (d, 1H, J=7.8 Hz), 4.01 (s, 2H), 3.57 (s, 3H), 3.30-3.15 (m, 2H), 3.06 (t, 2H, J=6.8 Hz), 2.70-2.50 (m, 3H), 2.35-1.80 (m, 8H); ESMS m/e: 429.3 (M+H)⁺.

### EXAMPLE 71

N-(3-{1-[4-(4-Chlorophenyl)-4-Oxobutyl]-4-Piperidinyl}Phenyl)Methanesulfonamide:

¹H NMR (400 MHz, CDCl₃) δ 7.95-6.96 (m, 8H), 3.48 (s, 3H), 3.28-2.90 (m, 6H), 2.80-2.57 (m, 3H), 2.38-1.86 (m, 6H); ESMS m/e: 435.2 (M+H)⁺.

### EXAMPLE 72

N-(3-{1-[4-(4-Chlorophenyl)-4-Oxobutyl]-4-Piperidinyl}Phenyl)Ethanesulfonamide:

¹H NMR (400 MHz, CDCl₃) δ 7.93 (d, 2H, J=8.2 Hz), 7.45 (d, 2H, J=8.2 Hz), 7.30-7.08 (m, 3H), 6.99 (d, 1H, J=7.6 Hz), 3.26-3.02 (m, 6H), 2.69-2.45 (m, 3H), 2.32-1.75 (m, 8H), 1.36 (t, 3H, J=7.4 Hz); ESMS m/e: 449.3 (M+H)⁺.

### EXAMPLE 73

N-{3-[1-(4-oxo-4-Phenylbutyl)-4-Piperidinyl]Phenyl}Acetamide:

¹H NMR (400 MHz, CDCl₃) δ 8.10-6.80 (m, 9H), 3.40-2.95 (m, 4H), 2.85-2.20 (m, 3H), 2.19 (s, 3H), 2.15-1.70 (m, 8H); ESMS m/e: 365.3 (M+H)⁺.

### EXAMPLE 74

2-Methyl-N-{3-[1-(4-oxo-4-Phenylbutyl)-4-Piperidinyl]Phenyl}Propanamide:

¹H NMR (400 MHz, CDCl₃) δ 7.99 (d, 2H, J=7.4 Hz), 7.57 (t, 1H, J=7.4 Hz), 7.48 (t, 2H, J=7.4 Hz), 7.45-7.20 (m, 2H), 7.24 (t, 1H, J=8.0 Hz), 6.94 (d, 1H, 8.0 Hz), 3.24-3.21 (m, 2H), 3.09 (t, 2H, J=7.0 Hz), 2.57-2.25 (m, 4H), 2.31-1.84 (m, 8H), 1.26 (d, 6H, J=7.2 Hz); ESMS m/e 393.3 (M+H)⁺.

### EXAMPLE 75

N-{3-[1-(4-oxo-4-Phenylbutyl)-4-Piperidinyl]Phenyl}-2-Phenylacetamide:

1H NMR (400 MHz, CDCl₃) δ 7.98 (d, 2H, J=7.6 Hz), 7.65-7.15 (m, 11H), 6.92 (d, 2H, J=7.2 Hz), 3.74 (s, 2H), 3.20-2.95 (m, 4H), 2.65-2.40 (m, 3H), 2.25-1.70 (m, 8H); ESMS m/e: 441.3 (M+H)⁺.

### EXAMPLE 76

2-(3-Methoxyphenyl)-N-{3-[1-(4-oxo-4-Phenylbutyl)-4-Piperidinyl]Phenyl}Acetamide:

¹H NMR (400 MHz, CDCl₃) δ 7.98 (d, 2H, J=7.6 Hz), 7.56 (t, 1H, J=7.62 Hz), 7.46 (t, 2H, J=7.6 Hz), 7.40 (s, 1H), 7.37-7.26 (m, 2H), 7.19 (t, 1H, J=7.8 Hz), 6.94-6.86 (m, 3H), 3.81 (s, 3H), 3.71 (s, 3H), 3.12-3.03 (m, 4H), 2.57-2.44 (m, 3H), 2.16-1.77 (m, 8H); ESMS m/e: 471.3 (M+H)⁺.

### EXAMPLE 77

N-(3-{1-[4-(2,4-Dimethoxyphenyl)-4-Oxobutyl]-4-Piperidinyl}Phenyl)Acetamide:

¹H NMR (400 MHz, CDCl₃) δ 7.82 (d, 1H, J=8.8 Hz), 7.54 (d, 1H, J=7.6 Hz), 7.33 (s, 1H), 7.22 (t, 1H, J=7.6 Hz), 6.93 (d, 1H, J=7.6 Hz), 6.53 (d, 1H, J=8.8 Hz), 6.46 (s, 1H), 3.90 (s, 3H), 3.86 (s, 3H), 3.48-3.27 (m, 2H), 3.05 (t, 2H, J=6.8 Hz), 2.90-2.68 (m, 2H), 2.65-2.38 (m, 3H), 2.25 (s, 3H), 2.18-1.80 (m, 6H); ESMS m/e: 425.3 (M+H)⁺.

### EXAMPLE 78

N-(3-{1-[4-(2,4-Dimethoxyphenyl)-4-Oxobutyl]-4-Piperidinyl}Phenyl)-2-Methylpropanamide:

¹H NMR (400 MHz, CDCl₃) δ 7.98 (d, 1H, J=8.6 Hz), 7.41-7.37 (m, 2H), 7.24 (t, 1H, J=7.8 Hz), 6.96 (d, 1H, J=7.8 Hz), 6.54 (d, 1H, J=8.6 Hz), 6.46 (s, 1H), 3.89 (s, 3H), 3.86 (s, 3H), 3.11-3.08 (m, 2H), 2.98 (t, 2H, J=7.2 Hz), 2.53-2.46 (m, 4H), 2.13-1.79 (m, 8H), 1.25 (d, 6H, J=6.8 Hz); ESMS m/e: 453.3 (M+H)⁺.

### EXAMPLE 79

N-(3-{¹-[4-(2,4-Dimethoxyphenyl)-4-Oxobutyl]-4-Piperidinyl}Phenyl)-2-Phenylacetamide:

¹H NMR (400 MHz, CDCl₃) δ 7.85 (m, 12H), 3.89 (s, 3H), 3.86 (s, 3H), 3.74 (s, 2H), 3.22-2.90 (m, 4H), 2.64-2.40 (m, 3H), 2.25-1.70 (m, 8H); ESMS m/e: 501.3 (M+H)⁺.

### EXAMPLE 80

N-(3-{1-[4-(2,4-Dimethoxyphenyl)-4-Oxobutyl]-4-Piperidinyl}Phenyl)-2-(3-Methoxyphenyl)Acetamide:

¹H NMR (400 MHz, CDCl₃) δ 7.82 (d, 1H, J=8.8 Hz), 7.48-7.15 (m, 5H), 6.95-6.80 (m, 3H), 6.58-6.45 (m, 2H), 3.89 (s, 3H), 3.86 (s, 3H), 3.81 (s, 3H), 3.72 (s, 2H), 3.25-2.95 (m, 4H), 2.65-2.40 (m, 3H), 2.30-1.95 (m, 4H), 1.93-1.72 (m, 4H); ESMS m/e: 531.3 (M+H)⁺.

### EXAMPLE 81

N-(3-{1-[4-oxo-4-(4-Phenoxyphenyl)Butyl]-4-Piperidinyl}Phenyl)Acetamide:

¹H NMR (400 MHz, CDCl₃) 6 8.15-6.75 (m, 13H), 3.30-2.80 (m, 4H), 2.75-2.10 (m, 5H), 2.03 (s, 3H), 2.00-1.60 (m, 6H); ESMS m/e: 457.3 (M+H)⁺.

### EXAMPLE 82

2-Methyl-N-(3-{1-[4-oxo-4-(4-Phenoxyphenyl)Butyl]-4-Piperidinyl}Phenyl)Propanamide:

¹H NMR (400 MHz, CDCl₃) δ 7.96 (d, 2H, J=8.8 Hz), 7.43-7.15 (m, 6H), 7.10-6.93 (m, 5H), 3.42-2.95 (m, 4H), 2.80-2.45 (m, 4H), 2.20-1.80 (m, 8H), 1.14 (d, 6H, J=6.8 Hz); ESMS m/e: 485.4 (M+H)⁺.

### EXAMPLE 83

2-(3-Methoxyphenyl)-N-(3-{1-[4-oxo-4-(4-Phenoxyphenyl)Butyl]-4-Piperidinyl}Phenyl)Acetamide:

¹H NMR (400 MHz, CDCl₃) δ 7.97 (d, 2H, J=8.8 Hz), 7.41-7.18 (m, 7H), 7.08-6.99 (m, 5H), 6.94-6.87 (m, 3H), 3.82 (s, 3H), 3.70 (s, 2H), 3.10-2.95 (m, 4H), 2.55-2.40 (m, 3H), 2.15-1.95 (m, 4H), 1.81-1.70 (m, 4H); ESMS m/e: 563.4 (M+H)⁺.

### EXAMPLE 84

N′-(3-{1-[4-(4-Chlorophenyl)-4-Oxobutyl]-4-Piperidinyl}Phenyl)-N,N-Dimethylsulfamide:

¹H NMR (400 MHz, CDCl₃) δ 7.93 (d, 2H, J=8.8 Hz), 7.44 (d, 2H, J=8.8 Hz), 7.27 (s, 1H), 7.25-7.10 (m, 2H), 6.94 (d, 1H, J=7.6 Hz), 3.30-3.10 (m, 2H), 3.04 (t, 2H, J=6.8 Hz), 2.83 (s, 6H), 2.68-2.45 (m, 3H), 2.30-1.75 (m, 8H); ESMS m/e: 464.3 (M+H)⁺.

### EXAMPLE 85

N-(3-{1-[4-oxo-4-(2-Thienyl)Butyl]-4-Piperidinyl}Phenyl)Acetamide:

¹H NMR (400 MHz, CDCl₃) δ 7.90-6.78 (m, 7H), 3.22-2.88 (m, 4H), 2.69-2.25 (m, 5H), 2.02 (s, 3H), 2.00-1.64 (m, 6H); ESMS m/e: 371.2 (M+H)⁺.

### EXAMPLE 86

N-(3-{1-[4-(4-Isopropylphenyl)-4-Oxobutyl]-4-Piperidinyl}Phenyl)Acetamide:

¹H NMR (400 MHz, CDCl₃) δ 8.00-6.78 (m, 8H), 3.15-2.98 (m, 4H), 2.77-2.15 (m, 4H), 2.03 (s, 3H), 2.00-1.62 (m, 8H), 0.927 (d, 6H, J=6.0 Hz); ESMS m/e: 407.3 (M+H)⁺.

### EXAMPLE 87

N-(3-{1-[4-(4-Methylphenyl)-4-Oxobutyl]-4-Piperidinyl}Phenyl)Acetamide: ¹H NMR (400 MHz, CDCl₃) δ 7.90-6.80 (m, 8H), 3.10-2.45 (m, 7H), 2.32 (S, 3H), 2.02 (s, 3H), 2.01-1.68 (m, 8H); ESMS m/e: 379.3 (M+H)⁺.

### EXAMPLE 88

N-(3-{1-[4-(4-Bromophenyl)-4-Oxobutyl]-4-Piperidinyl}Phenyl)Acetamide:

¹H NMR (400 MHz, CDCl₃) δ 7.90-6.80 (m, 8H), 3.30-3.05 (m, 4H), 2.70-2.45 (m, 3H), 2.05 (s, 3H), 1.98-1.65 (m, 8H); ESMS m/e: 444.0 (M+H)⁺.

### EXAMPLE 89

N-(3-{1-[4-(3,4-Dimethylphenyl)-4-Oxobutyl]-4-Piperidinyl}Phenyl)-2-Propanesulfonamide:

¹H NMR (400 MHz, CDCl₃) δ 7.75 (s, 1H), 7.71 (d, 1H, J=7.6 Hz), 7.27-7.00 (m, 5H), 3.32-3.24 (m, 3H), 3.10-3.02 (m, 2H), 2.78-2.50 (m, 3H), 2.32 (s, 6H), 2.19-1.84 (m, 8H), 1.39 (d, 6H, J=6.8 Hz); ESMS m/e: 457.4 (M+H)⁺.

### EXAMPLE 90

N-(3-{1-[4-oxo-4-(4-Phenoxyphenyl)Butyl]-4-Piperidinyl}Phenyl)-2-Propanesulfonamide:

¹H NMR (400 MHz, CDCl₃) δ 7.97 (d, 2H, J=7.6 Hz), 7.44 (t, 2H, J=7.6 Hz), 7.27-7.00 (m, 9H), 3.35-2.96 (m, 5H), 2.69-2.45 (m, 3H), 2.14-1.79 (m, 8H), 1.39 (d, 6H, J=6.8 Hz); ESMS m/e: 521.4 (M+H)⁺.

### EXAMPLE 91

N-(3-{1-[3-(4-Chlorophenyl)-3-Methoxypropyl]-4-Piperidinyl}Phenyl)-2-Methylpropanamide

A mixture of 3-methoxy-3-(p-chlorophenyl)-1-chloropropane (27.4 mg, 0.125 mmol), 2-methyl-N-[3-(4-piperidinyl)phenyl]propanamide (28.3 mg, 0.125 mmol), diisopropylethylamine (0.50 mL) and catalytic amount of tetrabutylammonium iodide in dioxane (2.0 mL) was stirred at 90° C. for 72 hrs. The reaction mixture was concentrated to a small volume and chromatographed using preparative TLC plates [2.5% of NH₃ (2.0 M in methanol) in CHCl₃] gave N-(3-{1-[3-(4-chlorophenyl)-3-methoxypropyl]-4-piperidinyl}phenyl)-2-methylpropanamide (39.5 mg, 73.8% yield) as a thick oil: ¹H NMR δ 7.48 (S, 1H), 7.34-7.3 (m, 2H), 7.25 (m, 4H), 6.96 (d, 1H, J=7.4 Hz), 4.20 (apparent dd, 1H, J=5.9, 7.6 Hz), 3.2 (s, 3H), 3.04 (d, 1H, J=10.1 Hz), 2.99 (d, 1H, J=10.1 Hz), 2.49 (h, 4H, J=6.6 Hz), 2.20-2.10 (m, 4H), 1.82 (m, 4H), 1.25 (d, 6H, J=7.1 Hz); ESMS m/e: 429.4 (M+H)⁺.

### EXAMPLE 92

N-(3-{1-[6-(1,3-Dioxo-1,3-Dihydro-2H-Isoindol-2-yl)Hexyl]-4-Piperidinyl}Phenyl)-2-Methylpropanamide:

The synthetic method is the same as described for 2-[6-(4-phenyl-1-piperidinyl)hexyl]-1H-isoindole-1,3(2H)-dione. N-(3-{1-[6-(1,3-dioxo-1,3-dihydro-2H-isoindol-2-yl)hexyl]-4-piperidinyl}phenyl)-2-methylpropanamide: 506 mg (56% yield); ¹H NMR (400 MHz, CDCl₃) δ 7.86-7.80 (m, 2H), 7.73-7.68 (m, 2H), 7.44 (s, 1H), 7.37 (d, 1H, J=8.3 Hz), 7.22 (t, 1H, J=7.7 Hz), 6.96 (d, 1H, J=7.7 Hz), 3.69 (t, 2H, J=7.2 Hz), 3.01 (apparent d, 2H, J=11.3 Hz), 2.58-2.40 (m, 2H), 2.33 (m, 2H) 1.98 (dt, 2H, J=3.2, 11.3 Hz), 1.84-1.64 (m, 4H), 1.51 (q, 2H, J=7.1 Hz), 1.43-1.30 (m, 6H), 1.24 (d, 6H, J=6.8 Hz); ESMS m/e: 476.4 (M+H)⁺.

### EXAMPLE 93

N-{3-[1-(3-Methoxy-3-Phenylpropyl)-4-Piperidinyliphenyl}-2-Methylpropanamide

A mixture of 3-methoxy-3-phenyl-1-chloropropane (23.1 mg, 0.126 mmol), 2-methyl-N-[3-(4-piperidinyl)phenyl]propanamide (28.3 mg, 0.126 mmol), diisopropylethylamine (0.50 mL) and catalytic amount of tetrabutylammonium iodide in dioxane (2.0 mL) was stirred at 90° C. for 72 hrs. Chromatography using silica preparative TLC plates [2.5% of NH₃ (2.0 M in methanol) in CHCl₃] gave N-{3-[1-(3-methoxy-3-phenylpropyl)-4-piperidinyl]phenyl}-2-methylpropanamide (45.4 mg, 91.2% yield) as a thick oil: 1H NMR (400 MHz, CDCl₃) δ 7.45 (S, 1H), 7.34-7.25 (m, 5H), 7.25 (m, 2H), 6.96 (d, 1H, J=7.4 Hz), 4.20 (apparent dd, 1H, J=5.9, 7.6 Hz), 3.2 (s, 3H), 3.04 (d, 1H, J=10.1 Hz), 2.99 (d, 1H, J=10.1 Hz), 2.49 (apparent sept, partially hidden, 4H, J=6.6 Hz), 2.3-2.1(m, 4H), 1.82 (m, 4H), 1.25 (d, 6H, J=7.1 Hz); ESMS m/e: 395.4 (M+H)⁺.

### EXAMPLE 94

N-(3-{1-[4-(1,3-Dioxo-1,3-Dihydro-2H-Isoindol-2-yl)Butyl]-4-Piperidinyl}Phenyl)-2-Methylpropanamide:

The synthetic method is the same as described for 2-[6-(4-phenyl-1-piperidinyl)hexyl]-1H-isoindole-1,3(2H)-dione. N-(3-{1-[4-(1,3-dioxo-1,3-dihydro-2H-isoindol-2-yl)butyl]-4-piperidinyl}phenyl)-2-methylpropanamide: 664 mg (74% yield); ¹H NMR (400 MHz, CDCl₃) δ 7.87-7.78 (m, 2H), 7.76-7.64 (m, 2H), 7.47 (s, 1H), 7.39 (d, 1H, J=7.6 Hz), 7.21 (t, 1H, J=8.1 Hz), 6.94 (d, 1H, J=7.6 Hz), 3.72 (t, 2H, J=6.8 Hz), 3.37-3.22 (m, 2H), 3.0 (apparent d, 2H, J=10.7 Hz), 2.75 (q, 2H, J=7.0 Hz), 2.64-2.33 (m, 4H), 1.99 (dt, 2H, J=2.6, 11.7 Hz), 1.86-1.65 (m, 2H), 1.63-1.50 (m, 2H), 1.23 and 1,21 (two d, 6H, J=5.5 Hz); ESMS m/e: 448.4 (M+H)⁺; Anal. Calc. for C₂₇H₃₄N₃ClO₃+0.4H₂O: C, 66.02; H, 7.14; N, 8.55. Found: C, 66.07; H, 6.78; N, 8.65.

### EXAMPLE 95

N-(3-{1-[4-(1,3-Dioxo-1,3-Dihydro-2H-Isoindol-2-yl)Butyl]-4-Piperidinyl}Phenyl)-2-Methylpropanamide:

The synthetic method is the same as described for 2-[6-(4-phenyl-1-piperidinyl)hexyl]-1H-isoindole-1,3(2H)-dione. N-(3-{1-[5-(1,3-dioxo-1,3-dihydro-2H-isoindol-2-yl)pentyl]-4-piperidinyl}phenyl)-2-methylpropanamide: 614 mg (64% yield); ¹H NMR (400 MHz, CDCl₃) δ 7.87-7.8 (m, 2H), 7.76-7.68 (m, 2H), 7.48 (s, 1H), 7.41 (d, 1H, J=7.6 Hz), 7.21 (t, 1H, J=7.6 Hz), 6.95 (d, 1H, J=7.6 Hz), 3.69 (t, 2H, J=7.2 Hz), 3.39-3.28 (m, 2H), 3.02 (apparent d, 2H, J=11.6 Hz), 2.78 (q, 2H, J=7.2 Hz), 2.64-2.52 (m, 1H), 2.52-2.40 (m, 1H), 2.40-2.31 (m, 2H), 2.01 (dt, 2H, J=3.7, 11.1 Hz), 1.85-1.64 (m, 2H), 1.58 (q, 2H, J=7.6 Hz), 1.45-1.32 (m, 2H), 1.23 (d, 6H, J=6.9 Hz); ESMS m/e: 462.4 (M+H)⁺; Anal. Calc. for C₂₈H₃₆N₃ClO₃: C, 67.52; H, 7.29; N, 8.44. Found: C, 67.04; H, 7.06; N, 8.38.

### EXAMPLE 96

2-Methyl-N-{3-[1-(4-Phenylbutyl)-4-Piperidinyl]Phenyl}Propanamide

A mixture of 2-methyl-N-[3-(4-piperidinyl)phenyl]propanamide (28.3 mg, 0.100 mmol), 4-phenyl-1-chlorobutane (21.1 mg, 0.125 mmol), diisopropylethylamine (0.50 mL), catalytic amount of tetrabutylammonium iodide and dioxane (2.0 mL) was heated at reflux temperature for 3 days. The reaction mixture was concentrated and chromatographed using preparative TLC plates [2.5% of NH₃ (2.0 M in methanol) in CHCl₃] afforded the product, 2-methyl-N-{3-[1-(4-phenylbutyl)-4-piperidinyl]phenyl}propanamide (9.50 mg, 25.1% yield) as a thick oil: ¹H NMR δ 7.37 (s, 1H), 7.29 (apparent d, 1H, J=7.9 Hz), 7.18 (m, 3H), 7.11 (m, 3H), 6.90 (apparent d, 1H, J=7.9 Hz), 3.02 (d, 2H, J=6.8 Hz), 2.41 (m, 4H, partially hidden), 2.01 (m, 2H), 1.78 (m, 4H), 1.57 (m, 4H), 1.18 (d, 6H, J=7.7 Hz); ESMS m/e: 379.4 (M+H)⁺.

### EXAMPLE 97

N-(3-{1-[3-(1,3-Dioxo-1,3-Dihydro-2H-Isoindol-2-yl)Propyl]-4-Piperidinyl}Phenyl)-2-Methylpropanamide:

The synthetic method is the same as described for 2-[6-(4-phenyl-1-piperidinyl)hexyl]-1H-isoindole-1,3(2H)-dione. N-(3-{1-[3-(1,3-dioxo-1,3-dihydro-2H-isoindol-2-yl)propyl]-4-piperidinyl}phenyl)-2-methylpropanamide: 810 mg (93% yield); ¹H NMR (400 MHz, CDCl₃) δ 7.87-7.82 (m, 2H), 7.73-7.68 (m, 2H), 7.57 (s, 1H), 7.36 (d, 1H, J=8.5 Hz), 7.18 (t, 1H, J=7.7 Hz), 6.79 (d, 1H, J=7.1 Hz), 3.78 (t, 2H, J=6.8 Hz), 3.06 (quintet, 2H, J=6 Hz), 2.95 (apparent d, 2H, J=12.2 Hz), 2.58-2.31 (m, 4H), 1.96-1.83 (m, 2H), 1.70 (apparent d, 2H, J=12.1 Hz), 1.52 (dt, 2H, J=3.5, 12.5 Hz), 1.03 (d, 6H, J=6.5 Hz); ESMS m/e: 434.4 (M+H)⁺.

### EXAMPLE 98

N-(3-{1-[(3S)-3-Hydroxy-3-Phenylpropyl]-4-Piperidinyl}Phenyl)-2-Methylpropanamide

A mixture of (S)-(−)-3-chloro-1-phenyl-1-propanol (0.426 g, 2.50 mmol, 99%ee), 2-methyl-N-[3-(4-piperidinyl)phenyl]propanamide (0.565 g, 2.00 mmol), diisopropylethylamine (1.29 g, 10.0 mmol), dioxane (5.0 mL) and catalytic amount of tetrabutylammonium iodide was stirred at 90° C. for 72 hrs. Chromatography using silica preparative TLC plates [2.5% of NH₃ (2.0 M in methanol) in CHCl₃] gave the desired product (306 mg, 39.3% yield) as a thick oil: ¹H NMR (400 MHz, CDCl₃) 6 7.46 (S, 1H), 7.42 (d, 4H, J=8.1 Hz), 7.35 (m, 1H), 7.30 (d, 1H, J=8.0 Hz), 7.23 (t, 1H, J=8.1 Hz), 7.12 (s, 1H), 6.96 (apparent dd, 1H, J=8.0 Hz), 5.0 (apparent dd, 1H, J=4.4, 8.3 Hz), 3.18 (apparent dd, 2H, J=2.5, 12.5 Hz), 2.74 (m, 2H), 2.50 (m, 2H), 2.3-2.1 (m, 6H), 1.8 (m, 2H), 1.25 (d, 6H, J=7.1 Hz); ESMS m/e: 389.2 (M+H)⁺.

### EXAMPLE 99

N-(3-{1-[3-Methoxy-3-(4-Methylphenyl)Propyl]-4-Piperidinyl}Phenyl)-2-Methylpropanamide

A mixture of 3-methoxy-3-(p-tolyl)-1-chloropropane (24.9 mg, 0.126 mmol), 2-methyl-N-[3-(4-piperidinyl)phenyl]propanamide (28.3 mg, 0.126 mmol), diisopropylethylamine (0.50 mL) and catalytic amount of tetrabutylammonium iodide in dioxane (2.0 mL) was stirred at 90° C. for 72 hrs. Chromatography using silica preparative TLC plates [2.5% of NH₃ (2.0 M in methanol) in CHCl₃] gave the desired product (10.9 mg, 21.2% yield) as a thick oil: 1H NMR (400 MHz, CDCl₃) δ 7.44 (s, 1H), 7.38 (m, 1H), 7.3-7.1 (m, 5H), 6.96 (d, 1H, J=7.4 Hz), 4.18 (apparent dd, 1H, J=5.6, 7.9 Hz), 3.24 (d, 1H, J=8.2 Hz), 3.2 (s, 3H), 3.11 (m, 2H, J=10.1 Hz), 2.49 (m, 4H), 2.35 (s, 3H), 2.3-2.1(m, 3H), 1.92 (d, 4H), 1.25 (d, 6H, J=7.1 Hz); ESMS m/e: 409.4 (M+H)⁺.

### EXAMPLE 100

N-{3-[1-(3-Isopropoxy-3-Phenylpropyl)-4-Piperidinyl]Phenyl)-2-Methylpropanamide

A mixture of 3-isopropyl-3′-phenyl-1-chloropropane (26.6 mg, 0.126 mmol), 2-methyl-N-[3-(4-piperidinyl)phenyl]propanamide (28.3 mg, 0.126 mmol), diisopropylethylamine (0.50 mL) and catalytic amount of tetrabutylammonium iodide in dioxane (2.0 mL) was stirred at 90° C. for 72 hrs. Chromatography using silica preparative TLC plates [2.5% of NH₃ (2.0 M in methanol) in CHCl₃] gave the desired product (14.1 mg, 26.5% yield) as a thick oil: ¹H NMR (400 MHz, CDCl₃) δ 7.46 (s, 1H), 7.43-7.37 (m, 2H), 7.33 (m, 3H), 7.23 (m, 2H), 6.95 (d, 1H, J=8.4 Hz), 4.46 (apparent dd, 1H, J=5.0, 8.3 Hz), 3.49 (apparent sept, 1H, J=7.1 Hz), 3.10 (s, 2H), 2.70 (m, 2H), 2.52 (apparent sept, partially hidden, 4H, J=6.6 Hz), 2.30-2.10 (m, 2H), 1.90-1.80 (d, 4H), 1.25 (d, 6H, J=7.1 Hz), 1.15 (d, 3H, J=6.4 Hz), 1.08 (d, 3H, J=6.4 Hz); ESMS m/e: 423.4 (M+H)⁺.

### EXAMPLE 101

N-(3-{1-[4,4-Bis(4-Fluorophenyl)Butyl]-4-Piperidinyl}Phenyl)-2-Methylpropanamide

A mixture of 4,4-bis(4-fluoro-phenyl)-1-chloro-butane (39.0 mg, 0.126 mmol), 2-methyl-N-[3-(4-piperidinyl)phenyl]propanamide (28.3 mg, 0.126 mmol), diisopropylethylamine (0.50 mL) and catalytic amount of tetrabutylammonium iodide in dioxane (2.0 mL) was stirred at 90° C. for 72 hrs. Chromatography using silica preparative TLC plates [2.5% of NH₃ (2.0 M in methanol) in CHCl₃] gave the desired product (15.9 mg, 25.2% yield) as a thick oil: ¹H NMR (400 MHz, CDCl₃) δ 8.02 (s, 1H), 7.41 (s, 1H), 7.3-7.15 (m, 4H), 7.10 (m, 3H), 6.89 (apparent t, 5H), 3.81 (t, 1H, J=7.8 Hz), 3.30 (s, 1H), 2.91 (d, 1H, J=12,5 Hz), 2.80 (m, 1H), 2.40 (m, 2H), 2.31 (t, 1H, J=8.0 Hz), 1.93 (apparent q, 3H, J=8.0 Hz), 1.72 (m, 3H), 1.40 (m, 2H), 1.20 (m, 2H), 1.15 (d, 6H, J=8.1 Hz); ESMS m/e: 491.4 (M+H)⁺

### EXAMPLE 102

N-{3-[1-(3-Methoxybenzyl)-4-Piperidinyl]Phenyl}-2-Methylpropanamide

A mixture of 2-methyl-N-[3-(4-piperidinyl)phenyl]propanamide (28.3 mg, 0.100 mmol), 3-methoxybenzyl chloride (19.6 mg, 0.125 mmol), diisopropylethylamine (0.50 mL), catalytic amount of tetrabutylammonium iodide and dioxane (2.0 mL). Chromatography using silica preparative TLC plates [2.5% of NH3 (2.0 M in methanol) in CHCl₃] afforded the desired product (10.2 mg, 27.9% yield) as a yellow solid: ¹H NMR (400 MHz, CDCl₃) δ 7.46 (s, 1H), 7.35 (apparent d, ¹H, J=8.3 Hz), 7.27-7.21 (m, 2H), 6.95 (apparent t, 3H, J=6.9 Hz), 6.82 (apparent dd, 1H, J=2.4, 8.3 Hz), 3.84 (m, 3H), 3.56 (s, 2H), 3.05 (d, 2H, J=10.5 Hz), 2.51 (apparent sept, partially hidden, 4H, J=7.2 Hz), 2.13 (apparent t, 2H, J=9.7 Hz), 1.88 (m, 2H), 1.25 (d, 6H, J=6.7 Hz); ESMS m/e: 367.3 (M+H)⁺.

### EXAMPLE 103

N-(3-{1-[3,5-Bis(Trifluoromethyl)Benzyl]-4-Piperidinyl}Phenyl)-2-Methylpropanamide

A mixture of 2-methyl-N-[3-(4-piperidinyl)phenyl]propanamide (28.3 mg, 0.100 mmol), 3,5-bis(trifluoromethyl)benzyl bromide (38.4 mg, 0.125 mmol), diisopropylethylamine (0.50 mL), catalytic amount of tetrabutylammonium iodide and dioxane (2.0 mL). Chromatography using silica preparative TLC plates [2.5% of NH₃ (2.0 M in methanol) in CHCl₃] gave the desired product (12.2 mg, 25.8% yield) as a thick oil: ¹H NMR (400 MHz, CDCl₃) δ 7.83 (s, 2H), 7.77 (s, 1H), 7.53 (s, 1H), 7.30-7.21 (m, 2H), 7.16 (s, 1H), 6.98 (apparent d, 1H, J=7.6 Hz), 3.62 (s, 2H), 2.94 (d, 2H, J=9.4 Hz), 2.51 (apparent sept, partially hidden, 2H, J=6.6 Hz), 2.14 (m, 2H), 1.82 (m, 4H), 1.25 (d, 6H, J=6.6 Hz); ESMS m/e: 473.2 (M+H)⁺.

### EXAMPLE 104

N-(3-{1-[(3R)-3-(3,4-Dimethoxyphenoxy)-3-Phenylpropyl]-4-Piperidinyl}Phenyl)-2-Methylpropanamide

Method A

4-{[(1R)-3-chloro-1-phenylpropyl]oxy}-1,2-dimethoxybenzene:

A mixture of 3,4-dimethoxyphenol (4.07 g, 26.4 mmol), (S)-(−)-3-chloro-phenyl-1-propanol (4.50 g, 26.4 mmol, 99%ee, Aldrich Chemical Co.), triphenylphosphine (6.92 g, 26.4 mmol) and diethyl azodicarboxylate (4.59 g, 26.4 mmol) in THF (110 mL) was stirred at room temperature for 24 h. The reaction mixture was concentrated in vacuo.

At this point, the residue can either be washed with pentane (x3) and the combined pentane extracts were concentrated and chromatographed (silica with hexanes-EtOAc 8:1 as the eluent) to give the desired product (as described as a general procedure by: Srebnik, M.; Ramachandran, P. V.; Brown, H. C. J. Org. Chem. 1988, 53, 2916-2920). This procedure was performed on a smaller scale reaction and only a 40% yield of the product was realized.

Alternatively, on a larger scale (26.4 mmol), the crude product was triturated with a small amount of dichloromethane and the precipitated triphenylphosphine oxide was filtered. The filtrate was concentrated and the crude product was chromatographed to give the desired product as a thick yellow oil (7.30 g, 88.9% yield): ¹H NMR (400 MHz, CDCl₃) δ 7.39-7.32 (m, 4H), 7.20 (m, 1H), 6.64 (d, 1H, J=8.7 Hz), 6.51 (d, 1H, J=2.7 Hz), 6.30 (dd, 1H, J=2.7, 8.7 Hz), 5.27 (apparent dd, 1H, J=4.5, 8.7 Hz), 3.79 (s, 3H), 3.77 (s, 3H), 3.61 (m, 1H), 2.45 (m, 1H), 2.20 (m, 1H), 1.80 (s, 1H); ESMS m/e: 307.11 (M+H)⁺.

N-(3-{1-[(3R)-3-(3,4-Dimethoxyphenoxy)-3-Phenylpropyl]-4-Piperidinyl}Phenyl)-2-Methylpropanamide:

A mixture of potassium carbonate (321 mg, 2.32 mmol), sodium iodide (522 mg, 3.48 mmol), 2-methyl-N-[3-(4-piperidinyl)phenyl]propanamide (570 mg, 2.32 mmol) and 4-{[(1R)-3-chloro-1-phenylpropyl]oxy}-1,2-dimethoxybenzene (712 mg, 2.32 mmol) in DMF (5.0 mL) was stirred at 100° C. for 3 hrs, at which time TLC indicated that the reaction was complete. The reaction mixture was poured into water (50 mL) and the aqueous layer was extracted with methylene chloride (3×30 mL). The combined organic extracts were washed with brine (30 mL), dried over MgSO₄ and concentrated under reduced pressure. The crude product was purified by Prep-TLC plates [2.5% of NH₃ (2.0 M in methanol) in CHCl₃] to afford the product (970 mg, 90.1%) as a thick oil.

Method B

Into a 25-mL RB-flask was added triphenylphosphine (9.80 mg, 0.0375 mmol), diethyl azodicarboxylate (5.22 mg, 0.0300 mmol), N-(3-[1-[(3S)-3-hydroxy-3-phenylpropyl]-4-piperidinyl}phenyl)-2-methylpropanamide (9.53 mg, 0.0250 mmol), 3,4-dimethoxyphenol (7.70 mg, 0.050 mmol) and THF (1.0 mL) at room temperature. The reaction mixture was stirred at room temperature overnight (16 hrs). The solvent was removed under reduced pressure and the residue was purified by preparative TLC plates [2.5% of NH₃ (2.0 M in methanol) in CHCl₃] to afford the desired product (4.4 mg, 34.1% yield) as a thick oil: ¹H NMR (400 MHz, CDCl₃) δ 7.46 (s, 1H), 7.40-7.30 (m, 4H), 7.25 (m, 3H), 6.97 (d, 1H, J=7.8 Hz), 6.64 (d, 1H, J=9.1 Hz), 6.51 (d, 1H, J=2.6 Hz), 6.29 (d, 1H, J=2.6, 9.1 Hz), 5.20 (apparent dd, 1H, J=4.4, 8.5 Hz), 3.80 (s, 3H), 3.77 (s, 3H), 3.23 (m, 2H), 2.77 (m, 2H), 2.5 (m, 2H), 2.3-2.1(m, 6H), 1.80 (m, 2H), 1.25 (d, 6H, J=7.9 Hz); ESMS m/e: 517.4 (M+H)⁺.

### EXAMPLE 105

2-Methyl-N-(3-{1-[(3S)-3-Phenoxy-3-Phenylpropyl]-4-Piperidinyl}Phenyl)Propanamide

A mixture of N-(3-{1-[(3R)-3-hydroxy-3-phenylpropyl]-4-piperidinyl}phenyl)-2-methylpropanamide (9.53 mg, 0.0250 mmol), phenol (4.70 mg, 0.050 mmol), triphenylphosphine (9.80 mg, 0.0375 mmol) and diethyl azodicarboxylate (5.22 mg, 0.0300 mmol) in THF (1.0 mL) was stirred at room temperature for 3 days. Chromatography using silica preparative TLC plates [2.5% of NH₃ (2.0 M in methanol) in CHCl₃] gave the desired product (2.7 mg, 23.6% yield) as a thick oil: ¹H NMR δ 7.46 (s, 2H), 7.40-7.30 (m, 4H), 7.25 (m, 3H), 7.20 (m, 2H), 6.97 (apparent d, 1H, J=7.4 Hz), 6.89 (apparent tt, 1H, J=0.8, 7.6 Hz), 6.84 (apparent dt, 1H, J=0.8, 8.0 Hz), 5.20 (apparent dd, 1H, J=4.4, 8.5 Hz), 3.35 (m, 2H), 2.91 (m, 2H), 2.60 (m, 2H), 2.30-2.10 (m, 6H), 1.90 (m, 2H), 1.25 (d, 6H, J=7.9 Hz); ESMS m/e: 457.4 (M+H)⁺;

### EXAMPLE 106

N-(3-{1-[(3S)-3-(4-Methoxyphenoxy)-3-Phenylpropyl]-4-Piperidinyl}Phenyl)-2-Methylpropanamide

A mixture of N-(3-{1-[(3R)-3-hydroxy-3-phenylpropyl]-4-piperidinyl}phenyl)-2-methylpropanamide (9.53 mg, 0.0250 mmol), 4-methoxyphenol (6.20 mg, 0.050 mmol), triphenylphosphine (9.80 mg, 0.0375 mmol) and diethyl azodicarboxylate (5.2 mg, 0.0300 mmol) in THF (1.0 mL) was stirred at room temperature for 3 days. Chromatography using silica preparative TLC plates [2.5% of NH₃ (2.0 M in methanol) in CHCl₃] gave the desired product (4.6 mg, 37.9% yield) as a thick oil. ¹H NMR (400 MHz, CDCl₃) δ 7.38-7.14 (m, 8H), 6.90 (apparent d, 1H, J=7.7 Hz), 6.72-6.46 (m, 4H), 5.09 (apparent dd, 1H, J=4.8, 8.1 Hz), 3.64 (s, 3H), 3.18 (m, 2H), 2.73 (m, 2H), 2.50 (m, 2H), 2.37-1.72 (m, 8H), 1.25 (d, 6H, J=7.4 Hz); ESMS m/e: 487.4 (M+H)⁺.

### EXAMPLE 107

N-(3-{1-[(3S)-3-(3-Chlorophenoxy)-3-Phenylpropyl3-4-Piperidinyl}Phenyl)-2-Methylpropanamide

A mixture of N-(3-{1-[(3R)-3-hydroxy-3-phenylpropyl]-4-piperidinyl}phenyl)-2-methylpropanamide (9.53 mg, 0.0250 mmol), 3-chlorophenol (6.40 mg, 0.050 mmol), triphenylphosphine (9.80 mg, 0.0375 mmol) and diethyl azodicarboxylate (5.22 mg, 0.0300 mmol) in THF (1.0 mL) was stirred at room temperature for 3 days. Chromatography using silica preparative TLC plates [2.5% of NH₃ (2.0 M in methanol) in CHCl₃] gave the desired product (4.9 mg, 40.0% yield) as a thick oil: ¹H NMR (400 MHz, CDCl₃) δ 7.39 (s, 1H), 7.35-7.10 (m, 7H), 7.02 (t, 1H, J=8.0 Hz), 6.90 (d, 1H, J=7.6 Hz), 6.84-6.75 (m, 2H), 6.65 (m, 1H), 5.09 (apparent dd, 1H, J=4.99, 8.1 Hz), 3.10 (m, 2H), 2.60 (m, 2H), 2.50 (m, 2H), 2.30-1.70 (m, 8H), 1.18 (d, 6H, J=6.8 Hz); ESMS m/e: 491.4 (M+H)⁺.

### EXAMPLE 108

N-(3-{1-[(3S)-3-(4-Chlorophenoxy)-3-Phenylpropyl]-4-Piperidinyl}Phenyl)-2-Methylpropanamide

A mixture of N-(3-{1-[(3R)-3-hydroxy-3-phenylpropyl]-4-piperidinyl}phenyl)-2-methylpropanamide (9.53 mg, 0.0250 mmol), 4-chlorophenol (6.40 mg, 0.050 mmol), triphenylphosphine (9.80 mg, 0.0375 mmol) and diethyl azodicarboxylate (5.22 mg, 0.0300 mmol) in THF (1.0 mL) was stirred at room temperature for 3 days. Chromatography using silica preparative TLC plates [2.5% of NH₃ (2.0 M in methanol) in CHCl₃] gave the desired product (3.3 mg, 26.9% yield) as a thick oil: ¹H NMR δ 7.36 (s, 1H), 7.35-7.22 (m, 7H), 7.12 (m, 2H), 6.97 (apparent d, 1H, J=7.2 Hz), 6.77 (m, 2H), 5.23 (m, 1H), 3.18 (m, 2H), 2.70 (m, 2H), 2.50 (m, 2H), 2.40-1.80 (m, 8H), 1.25 (d, 6H, J=6.8 Hz); ESMS m/e: 491.4 (M+H)⁺.

### EXAMPLE 109

2-Methyl-N-[3-(1-((3S)-3-Phenyl-3-[4-(Trifluoromethyl)Phenoxy]Propyl)-4-Piperidinyl)Phenyl]Propanamide

A mixture of N-(3-{1-[(3R)-3-hydroxy-3-phenylpropyl]-4-piperidinyl}phenyl)-2-methylpropanamide (9.53 mg, 0.0250 mmol), 4-trifluoromethylphenol (8.100 mg, 0.050 mmol), triphenylphosphine (9.8 mg, 0.0375 mmol) and diethyl azodicarboxylate (5.22 mg, 0.0300 mmol) in THF (1.0 mL) was stirred at room temperature for 3 days. Chromatography using silica preparative TLC plates [2.5% of NH₃ (2.0 M in methanol) in CHCl₃] gave the desired product (5.10 mg, 38.9% yield) as a thick oil: ¹H NMR δ 8.06 (s, 1H), 7.49 (s, 1H), 7.44 (apparent d, 2H, J=0.6 Hz), 7.38-7.30 (m, 4H), 7.30-7.20 (m, 3H), 6.96 (apparent d, 1H, J=7.6 Hz), 6.91 (apparent d, 2H, J=8.6 Hz), 5.34 (m, 1H), 3.19 (m, 2H), 2.72 (m, 2H), 2.53 (m, 2H), 2.40-1.80 (m, 8H), 1.25 (d, 6H, J=6.8 Hz); ESMS m/e: 525.4 (M+H)⁺.

### EXAMPLE 110

N-(3-{1-[(3R)-3-(2,5-Difluorophenoxy)-3-Phenylpropyl]-4-Piperidinyl}Phenyl)-2-Methylpropanamide

A mixture of N-(3-{1-[(3R)-3-hydroxy-3-phenylpropyl]-4-piperidinyl}phenyl)-2-methylpropanamide (9.53 mg, 0.0250 mmol), 2,5-difluorophenol (6.50 mg, 0.050 mmol), triphenylphosphine (9.80 mg, 0.0375 mmol) and diethyl azodicarboxylate (5.22 mg, 0.0300 mmol) in THF (1.0 mL) was stirred at room temperature for 3 days. Chromatography using silica preparative TLC plates [2.5% of NH₃ (2.0 M in methanol) in CHCl₃] gave the desired product (3.60 mg, 29.3% yield) as a thick oil: ¹H NMR δ 7.46 (s, 1H), 7.40-7.32 (m, 4H), 7.31-7.20 (m, 2H), 7.17 (s, 1H), 7.01-6.92 (m, 2H), 6.65-6.42 (m, 2H), 5.27 (m, 1H), 3.13 (m, 2H), 2.64 (m, 2H), 2.51 (m, 2H), 2.28-1.80 (m, 8H), 1.25 (d, 6H, J=7.1 Hz); ESMS m/e: 493.4 (M+H)⁺.

### EXAMPLE 111

N-(3-{1-[(3R)-3-(3,4-Dichlorophenoxy)-3-Phenylpropyl]-4-Piperidinyl}Phenyl)-2-Methylpropanamide

A mixture of N-(3-{1-[(3S)-3-hydroxy-3-phenylpropyl]-4-piperidinyl}phenyl)-2-methylpropanamide (9.53 mg, 0.0250 mmol), 3,4-dichlorophenol (8.20 mg, 0.050 mmol), triphenylphosphine (9.80 mg, 0.0375 mmol) and diethyl azodicarboxylate (5.22 mg, 0.0300 mmol) in THF (1.0 mL) was stirred at room temperature for 3 days. Chromatography using silica preparative TLC plates [2.5% of NH3 (2.0 M in methanol) in CHCl₃] gave the desired product (5.20 mg, 39.7% yield) as a thick oil: ¹H NMR δ 7.70-7.63 (m, 2H), 7.55 (m, 1H), 7.47-7.43 (m, 3H), 7.40-7.19 (m, 3H), 7.00-6.50 (m, 2H), 6.69 (dd, 1H, J=2.2, 8.8 Hz), 5.25 (m, 1H), 3.20 (m, 2H), 2.70 (m, 2H), 2.53 (m, 2H), 2.40-2.20 (m, 4H), 2.10-1.80 (m, 4H), 1.25 (d, 6H, J=7.1 Hz); ESMS m/e: 525.4 (M+H)⁺.

### EXAMPLE 112

2-Methyl-N-(3-{1-[(3R)-3-Phenoxy-3-Phenylpropyl]-4-Piperidinyl}Phenyl)Propanamide

A mixture of N-(3-{1-[(3S)-3-hydroxy-3-phenylpropyl]-4-piperidinyl}phenyl)-2-methylpropanamide (9.53 mg, 0.0250 mmol), phenol (4.70 mg, 0.050 mmol), triphenylphosphine (9.80 mg, 0.0375 mmol) and diethyl azodicarboxylate (5.22 mg, 0.0300 mmol) in THF (1.0 mL) was stirred at room temperature for 3 days. Chromatography using silica preparative TLC plates [2.5% of NH₃ (2.0 M in methanol) in CHCl₃] gave the desired product (4.1 mg, 36.0% yield) as a thick oil: ¹H NMR (400 MHz, CDCl₃)δ 7.45 (s, 1H), 7.40-7.15 (m, 10H), 6.97 (d, 1H, J=7.6 Hz), 6.88-6.82 (m, 2H), 5.26 (m, 1H), 3.18 (m, 2H), 2.75 (m, 2H), 2.53 (m, 2H), 2.40-2.10 (m, 4H), 2.10-1.80 (m, 4H), 1.25 (d, 6H, J=6.9 Hz); ESMS m/e: 457.4 (M+H)⁺.

### EXAMPLE 113

N-(3-{1-[(3R)-3-Hydroxy-3-Phenylpropyl]-4-Piperidinyl}Phenyl)-2-Methylpropanamide

Method A

Into a 25-mL RB-flask was added (R)-(+)-3-chloro-1-phenyl-1-propanol (0.545 g, 3.19 mmol, 99%ee, Aldrich Chemical Co.), 2-methyl-N-[3-(4-piperidinyl)phenyl]propanamide (0.748 g, 3.04 mmol), potassium carbonate (0.420 g, 3.04 mmol) and sodium iodide (0.684 g, 4.56 mmol) and DMF (6.0 mL) at room temperature. After stirring at 100° C. for 3 hrs, the TLC showed the reaction was complete. The reaction mixture was poured into water (50 mL) and the aqueous layer was extracted with methylene chloride (3×20 mL). The combined organic extracts were washed with brine (20 mL), dried over Na₂SO₄ and concentrated under reduced pressure. The residue was purified by flash chromatography (1:1=hexane: ethyl acetate with 1% isopropylamine) to afford the desired product (1.09 g, 94.3% yield) as light-yellow solid: ¹H NMR (400 MHz, CDCl₃) δ 8.10 (s, 1H), 7.46-7.35 (m, 6H), 7.27 (m, 2H), 6.98 (apparent d, 1H, J=7.6 Hz), 5.02 (apparent dd, 1H, J=4.4, 8.1 Hz), 3.18 (apparent dd, 2H, J=2.5, 12.5 Hz), 2.74 (m, 2H), 2.50 (m, 2H), 2.30-2.10 (m, 6H), 1.80 (m, 2H), 1.25 (d, 6H, J=7.1 Hz); ESMS m/e: 381.2 (M+H)⁺. The hydrochloric salt was prepared by addition of a slight excess of 1 N HCl in ether (1.2 eq.) to a solution of the free base in dichloromethane. The solvent was removed under reduced pressure, the residue was washed with ether and dried under reduced pressure: Anal. Calc. for C₂₄H₃₂N₂O₂+HCl+0.8H₂O: C, 66.82; H, 8.08; N, 6.49; Cl, 8.22. Found: C, 66.90; H, 7.78; N, 6.63; Cl, 8.52.

Method B

Into a 25-mL RB-flask was added (R)-(+)-3-chloro-1-phenyl-1-propanol (0.426 g, 2.50 mmol), 2-methyl-N-[3-(4-piperidinyl)phenyl]propanamide (0.565 g, 2.00 mmol), diisopropylethylamine (1.29 g, 10.0 mmol), dioxane (5.0 mL) and catalytic amount of tetrabutylammonium iodide at room temperature. After stirring at 90° C. for 72 hrs, the reaction mixture was poured into water (50 mL) and the aqueous layer was extracted with methylene chloride (3×20 mL). The combined organic extracts were washed with brine (20 mL), dried over Na₂SO₄ and concentrated under reduced pressure. The residue was purified by preparative TLC plates (1:5:100=isopropylamine:methanol:ethyl acetate) to afford the desired product (0.260 g, 34.2% yield) as light-yellow solid.

### EXAMPLE 114

N-(3-{1-[(3S)-3-(4-cyanophenoxy)-3-phenylpropyl]-4-piperidinyl}phenyl)-2-methylpropanamide

A mixture of N-(3-{1-[(3R)-3-hydroxy-3-phenylpropyl]-4-piperidinyl}phenyl)-2-methylpropanamide (5.20 mg, 0.0137 mmol), 4-cyanophenol (100 mg), triphenylphosphine (30.0 mg, 0.115 mmol) and diethyl azodicarboxylate (7.42 mg, 0.0426 mmol) in THF (0.50 mL) was stirred at room temperature for 3 days. Chromatography using silica preparative TLC plates [2.5% of NH₃ (2.0 M in methanol) in CHCl₃] gave the desired product (4.70 mg, 71.3% yield) as a thick oil: ¹H NMR (400 MHz, CDCl₃) δ 7.54 (m, 2H), 7.48 (d, 2H, J=8.4 Hz), 7.30-7.20 (m, 3H), 7.20 (m, 3H), 6.97 (apparent d, 1H, J=8.4 Hz), 6.92 (apparent d, 2H, J=8.4 Hz), 5.36 (apparent dd, 1H, J=3.9, 7.6 Hz), 3.12 (m, 2H), 2.61 (m, 2H), 2.53 (apparent sept, partially hidden, 2H, J=7.6 Hz), 2.30-2.10 (m, 6H), 1.82 (m, 2H), 1.25 (d, 6H, J=6.8 Hz); ESMS m/e: 482.2 (M+H)⁺.

### EXAMPLE 115

N-(3-{1-[(3S)-3-(4-Fluorophenoxy)-3-Phenylpropyl]-4-Piperidinyl}Phenyl)-2-Methylpropanamide

A mixture of N-(3-{1-[(3R)-3-hydroxy-3-phenylpropyl]-4-piperidinyl}phenyl)-2-methylpropanamide (5.20 mg, 0.0137 mmol), 4-fluorophenol (100 mg), triphenylphosphine (30.0 mg, 0.115 mmol) and diethyl azodicarboxylate (7.42 mg, 0.0426 mmol) in THF (0.50 mL) was stirred at room temperature for 3 days. Chromatography using silica preparative TLC plates [2.5% of NH₃ (2.0 M in methanol) in CHCl₃] gave the desired product (4.20 mg, 64.7% yield) as a thick oil: ¹H NMR (400 MHz, CDCl₃) δ 7.40 (m, 2H), 7.30-7.20 (m, 5H), 7.20 (m, 3H), 6.97 (apparent d, 1H, J=7.7 Hz), 6.87 (m, 1H), 6.76 (m, 1H), 5.26 (apparent dd, 1H, J=4.0, 8.1 Hz), 3.09 (m, 2H), 2.66 (m, 2H), 2.51 (m, 2H), 2.3-2.1 (m, 6H), 1.82 (m, 2H), 1.25 (d, 6H, overlapped); ESMS m/e: 475.2 (M+H)⁺.

### EXAMPLE 116

N-(3-{1-[(3S)-3-(4-Bromophenoxy)-3-Phenylpropyl]-4-Piperidinyl}Phenyl)-2-Methylpropanamide

A mixture of N-(3-{1-[(3R)-3-hydroxy-3-phenylpropyl]-4-piperidinyl}phenyl)-2-methylpropanamide (5.20 mg, 0.0137 mmol), 4-bromophenol (100 mg), triphenylphosphine (30.0 mg, 0.115 mmol) and diethyl azodicarboxylate (7.42 mg, 0.0426 mmol) in THF (0.50 mL) was stirred at room temperature for 3 days. Chromatography using silica preparative TLC plates [2.5% of NH₃ (2.0 M in methanol) in CHCl₃] the desired product (0.70 mg, 9.6% yield) as a thick oil: ¹H NMR (400 MHz, CDCl₃) δ 8.06 (s, 1H), 7.48 (m, 2H), 7.30-7.20 (m, 5H), 7.20 (m, 3H), 6.97 (apparent d, 1H, J=8.5 Hz), 6.73 (apparent d, 2H, J=8.5 Hz), 5.22 (apparent dd, 1H, J=4.9, 7.8 Hz), 3.15 (m, 2H), 2.65 (m, 2H), 2.51 (apparent sept, partially hidden, 2H, J=7.6 Hz), 2.30-2.10 (m, 6H), 1.82 (m, 2H), 1.25 (d, 6H, J=6.8 Hz); ESMS m/e: 535.1 (M+H)⁺.

### EXAMPLE 117

N-(3-{1-[(3S)-3-(3-Methoxyphenoxy)-3-Phenylpropyl]-4-Piperidinyl}Phenyl)-2-Methylpropanamide

A mixture of N-(3-{1-[(3R)-3-hydroxy-3-phenylpropyl]-4-piperidinyl}phenyl)-2-methylpropanamide (5.20 mg, 0.0137 mmol), 3-methoxyphenol (100 mg), triphenylphosphine (30.0 mg, 0.115 mmol) and diethyl azodicarboxylate (7.42 mg, 0.0426 mmol) in THF (0.50 mL) was stirred at room temperature for 3 days. Chromatography using silica preparative TLC plates [2.5% of NH₃ (2.0 M in methanol) in CHCl₃] gave the desired product (3.1 mg, 46.6% yield) as a thick oil: 1H NMR (400 MHz, CDCl₃) δ 7.47 (d, 1H, J=6.7 Hz), 7.42 (s, 1H), 7.3-7.20 (m, 3H), 7.20 (m, 3H), 7.07 (t, 1H, J=8.4 Hz), 6.97 (apparent d, 1H, J=6.7 Hz), 6.40 (m, 3H), 5.27 (apparent dd, 1H, J=5.3, 8.0 Hz), 3.74 (s, 3H), 3.38 (m, 2H), 2.93 (m, 2H), 2.61 (s, 1H), 2.53 (apparent sept, partially hidden, 1H, J=6.5 Hz), 2.30-2.10 (m, 6H), 1.82 (m, 2H), 1.25 (d, 6H, J=6.9 Hz); ESMS m/e: 487.3 (M+H)⁺.

### EXAMPLE 118

N-(3-{1-[(3S)-3-(4-Cyano-2-Methoxyphenoxy)-3-Phenylpropyl]-4-Piperidinyl}Phenyl)-2-Methylpropanamide

A mixture of N-(3-{1-[(3R)-3-hydroxy-3-phenylpropyl]-4-piperidinyl}phenyl)-2-methylpropanamide (5.20 mg, 0.0137 mmol), 2-methoxy-4-cyanophenol (100 mg), triphenylphosphine (30.0 mg, 0.115 mmol) and diethyl azodicarboxylate (7.42 mg, 0.0426 mmol) in THF (0.50 mL) was stirred at room temperature for 3 days. Chromatography using silica preparative TLC plates [2.5% of NH₃ (2.0 M in methanol) in CHCl₃] gave the desired product (5.50 mg, 76.5% yield) as a thick oil: ¹H NMR (400 MHz, CDCl₃) δ 7.51 (s, 1H), 7.38 (s, 1H), 7.37 (d, 2H, J=2.4 Hz), 7.20 (m, 4H), 7.10 (d, 1H, J=2.4 Hz), 7.08 (s, 1H), 6.99 (apparent d, 1H, J=8.3 Hz), 6.76 (apparent d, 1H, J=8.3 Hz), 5.43 (apparent dd, 1H, J=5.1, 8.0 Hz), 3.91 (s, 3H), 3.34 (m, 2H), 2.63 (m, 2H), 2.63 (s, 1H), 2.53 (apparent sept, partially hidden, 1H, J=7.7 Hz), 2.30-2.10 (m, 6H), 1.82 (m, 2H), 1.28 (d, 6H, J=6.8 Hz); ESMS m/e: 512.2 (M+H)⁺.

### EXAMPLE 119

N-(3-{1-[(3S)-3-(5-Acetyl-2-Methoxyphenoxy)-3-Phenylpropyl]-4-Piperidinyl}Phenyl)-2-Methylpropanamide

A mixture of N-(3-{1-[(3R)-3-hydroxy-3-phenylpropyl]-4-piperidinyl}phenyl)-2-methylpropanamide (5.20 mg, 0.0137 mmol), 2-methoxy-5-acetylphenol (100 mg), triphenylphosphine (30.0 mg, 0.115 mmol) and diethyl azodicarboxylate (7.42 mg, 0.0426 mmol) in THF (0.50 mL) was stirred at room temperature for 3 days. Chromatography using silica preparative TLC plates [2.5% of NH₃ (2.0 M in methanol) in CHCl₃] gave the desired product (1.60 mg, 22.2% yield) as a thick oil: ¹H NMR (400 MHz, CDCl₃) δ 7.52 (d, 2H, J=2.4 Hz), 7.3-7.2 (m, 5H), 7.20 (m, 3H), 6.97 (apparent d, 1H, J=6.7 Hz), 6.69 (apparent d, 1H, J=8.0 Hz), 5.47 (apparent dd, 1H, J=4.3, 7.8 Hz), 3.95 (s, 3H), 3.38 (m, 2H), 2.93 (m, 2H), 2.61 (s, 1H), 2.53 (apparent sept, partially hidden, 1H, J=7.6 Hz), 2.50 (s, 3H), 2.30-2.10 (m, 6H), 1.82 (m, 2H), 1.25 (d, 6H, J=6.8 Hz); ESMS m/e: 529.6 (M+H)⁺.

### EXAMPLE 120

N-(3-{1-[(3R)-3-(2-Acetylphenoxy)-3-Phenylpropyl]-4-Piperidinyl}Phenyl)-2-Methylpropanamide

A mixture of N-(3-{1-[(3S)-3-hydroxy-3-phenylpropyl]-4-piperidinyl}phenyl)-2-methylpropanamide (5.2 mg, 0.0137 mmol), 2-acetylphenol (100 mg), triphenylphosphine (30.0 mg, 0.115 mmol) and diethyl azodicarboxylate (7.42 mg, 0.0426 mmol) in THF (0.50 mL) was stirred at room temperature for 3 days. Chromatography using silica preparative TLC plates [2.5% of NH₃ (2.0 M in methanol) in CHCl₃] gave the desired product (1.70 mg, 24.9% yield) as a thick oil: ¹H NMR (400 MHz, CDCl₃) δ 7.65 (m, 1H), 7.55 (s, 1H), 7.30-7.20 (m, 5H), 7.20 (m, 3H), 6.97 (m, 2H), 6.76 (apparent d, 1H), 5.49 (apparent dd, 1H, J=4.3, 8.0 Hz), 3.38 (m, 2H), 2.93 (m, 2H), 2.71 (s, 3H), 2.60 (s, 1H), 2.53 (apparent sept, partially hidden, 1H, J=7.6 Hz), 2.30-2.10 (m, 6H), 1.82 (m, 2H), 1.25 (d, 6H, J=6.9 Hz); ESMS m/e: 498.8 (M+).

### EXAMPLE 121

N-[3-(1-{(3R)-3-[2-Fluoro-5-(Trifluoromethyl)Phenoxy]-3-Phenylpropyl}-4-Piperidinyl)Phenyl]-2-Methylpropanamide

A mixture of N-(3-{1-[(3S)-3-hydroxy-3-phenylpropyl]-4-piperidinyl}phenyl)-2-methylpropanamide (5.20 mg, 0.0137 mmol), 2-fluoro-5-trifluoromethylphenol (100 mg), triphenylphosphine (30.0 mg, 0.115 mmol) and diethyl azodicarboxylate (7.42 mg, 0.0426 mmol) in THF (0.50 mL) was stirred at room temperature for 3 days. Chromatography using silica preparative TLC plates [2.5% of NH₃ (2.0 M in methanol) in CHCl₃] gave the desired product (2.50 mg, 33.7% yield) as a thick oil: ¹H NMR (400 MHz, CDCl₃) δ 8.07 (s, 1H), 7.67 (m, 1H), 7.54 (m, 1H), 7.45 (m, 2H), 7.30-7.10 (m, 6H), 7.14 (d, 1H, J=7.4 Hz), 6.97 (apparent d, 1H, J=7.7 Hz), 5.37 (apparent dd, 1H, J=5.0, 8.5 Hz), 3.4 (m, 2H), 2.8 (m, 2H), 2.6 (s, 1H), 2.53 (apparent sept, partially hidden, 1H, J=7.4 Hz), 2.30-2.10 (m, 6H), 1.80 (m, 2H), 1.25 (d, 6H, J=7.1 Hz, overlapped); ESMS m/e: 542.6 (M⁺), 543.54 (M+H)⁺.

### EXAMPLE 122

N-[3-(1-{(3S)-3-[2-Fluoro-5-(Trifluoromethyl)Phenoxy]-3-Phenylpropyl}-4-Piperidinyl)Phenyl]-2-Methylpropanamide

A mixture of N-(3-{1-[(3R)-3-hydroxy-3-phenylpropyl]-4-piperidinyl}phenyl)-2-methylpropanamide (5.20 mg, 0.0137 mmol), 2-fluoro-5-trifluoromethylphenol (100 mg), triphenylphosphine (30.0 mg, 0.115 mmol) and diethyl azodicarboxylate (7.42 mg, 0.0426 mmol) in THF (0.50 mL) was stirred at room temperature for 3 days. Chromatography using silica preparative TLC plates [2.5% of NH₃ (2.0 M in methanol) in CHCl₃] gave the desired product (3.00 mg, 40.4% yield) as a thick oil: ¹H NMR (400 MHz, CDCl₃) δ 8.06 (s, 1H), 7.67 (m, 2H), 7.55 (m, 2H), 7.50-7.40 (m, 3H), 7.30-7.10 (m, 3H), 7.17 (d, 1H, J=8.9 Hz), 7.07 (apparent d, 1H, J=6.7 Hz), 6.97 (apparent d, 1H, J=7.8 Hz), 5.37 (apparent dd, 1H, J=4.2, 8.1 Hz), 3.37 (m, 2H), 2.93 (m, 2H), 2.63 (s, 1H), 2.50 (apparent sept, partially hidden, 1H, J=7.9 Hz), 2.30-2.10 (m, 6H), 1.85 (m, 2H), 1.25 (d, 6H, J=6.9 Hz); ESMS m/e: 542.7 (M+H)⁺.

### EXAMPLE 123

N-(3-{1-[(3S)-3-(2,5-Difluorophenoxy)-3-Phenylpropyl]-4-Piperidinyl}Phenyl)-2-Methylpropanamide

A mixture of N-(3-{1-[(3R)-3-hydroxy-3-phenylpropyl]-4-piperidinyl}phenyl)-2-methylpropanamide (5.20 mg, 0.0137 mmol), 2,5-difluorophenol (100 mg), triphenylphosphine (30.0 mg, 0.115 mmol) and diethyl azodicarboxylate (7.42 mg, 0.0426 mmol) in THF (0.50 mL) was stirred at room temperature for 3 days. Chromatography using silica preparative TLC plates [2.5% of NH₃ (2.0 M in methanol) in CHCl₃] gave the desired product (2.70 mg, 40.1% yield) as a thick oil: ¹H NMR (400 MHz, CDCl₃) δ 7.46 (s, 1H), 7.40-7.30 (m, 4H), 7.20 (m, 2H), 7.17 (s, 1H), 6.97 (m, 2H), 6.58 (m, 1H), 6.51 (m, 1H), 5.27 (apparent dd, 1H, J=5.1, 8.2 Hz), 3.13 (apparent d, J=9.7 Hz, 2H), 2.64 (m, 2H), 2.51 (m, 2H), 2.34 (apparent sept, partially hidden, J=7.1 Hz, 1H), 2.17 (m, 3H), 1.90-1.80 (m, 4H), 1.25 (d, 6H, J=7.1 Hz); ESMS m/e: 493.1 (M+H)⁺.

### EXAMPLE 124

N-(3-{1-[(3R)-3-(3-Chlorophenoxy)-3-Phenylpropyl]-4-Piperidinyl}Phenyl)-2-Metrylpropanamide

A mixture of N-(3-{1-[(3S)-3-hydroxy-3-phenylpropyl]-4-piperidinyl}phenyl)-2-methylpropanamide (5.20 mg, 0.0137 mmol), 3-chlorophenol (100 mg), triphenylphosphine (30.0 mg, 0.115 mmol) and diethyl azodicarboxylate (7.42 mg, 0.0426 mmol) in THF (0.50 mL) was stirred at room temperature for 3 days. Chromatography using silica preparative TLC plates [2.5% of NH₃ (2.0 M in methanol) in CHCl₃] gave the desired product (2.4 mg, 35.8% yield) as a thick oil: ¹H NMR (400 MHz, CDCl₃) δ 7.30 (m, 2H), 7.30-7.20 (m, 3H), 7.20 (m, 3H), 6.90 (apparent d, 1H, J=7.7 Hz), 6.71 (apparent d, 1H, J=2.9 Hz), 6.69 (apparent t, 1H, J=2.9 Hz), 6.67 (apparent t, 1H, J=2.9 Hz), 6.65 (apparent d, 1H, J=2.9 Hz), 5.09 (apparent dd, 1H, J=4.8, 8.1 Hz), 3.18 (m, 2H), 2.73 (m, 2H), 2.50 (apparent sept, partially hidden, 2H, J=7.1 Hz), 2.30-2.10 (m, 6H), 1.89 (m, 2H), 1.25 (d, 6H, overlapped); ESMS m/e: 491.1 (M+H)⁺.

### EXAMPLE 125

(1S)-3-{4-[3-(Isobutyrylamino)Phenyl]-1-Piperidinyl}-1

Into a 25-mL RB-flask was added N-(3-{1-[(3S)-3-hydroxy-3-phenylpropyl]-4-piperidinyl}phenyl)-2-methylpropanamide (5.20 mg, 0.0137 mmol), 1-naphthalenecarbonyl chloride (100 mg), diisopropylethylamine (0.30 mL) in THF (0.50 mL) at room temperature. After stirring for 16 hrs at room temperature, the reaction mixture was concentrated under reduced pressure. The residue was purified using preparative TLC plates [2.5% of NH₃ (2.0 M in methanol) in CHCl₃] gave the desired product (4.70 mg, 71.3% yield) as a thick oil: ¹H NMR (400 MHz, CDCl₃) δ 8.90 (d, 1H, J=8.9 Hz), 8.28 (apparent dd, 1H, J=1.5, 7.2 Hz), 8.03 (d, 1H, J=8.7 Hz), 7.88 (dm, 2H, J=8.7 Hz), 7.60-7.48 (m, 7H), 7.40-7.32 (m, 3H), 7.25 (m, 1H), 6.90 (apparent d, 1H, J=7.4 Hz), 6.18 (apparent dd, 1H, J=5.7, 7.8 Hz), 3.42 (m, 2H), 2.84 (m, 2H), 2.53 (m, 2H), 2.44 (apparent sept, partially hidden, 4H, J=7.5 Hz), 2.30-2.10 (m, 2H), 1.82 (m, 2H), 1.25 (d, 6H, J=6.8 Hz); ESMS m/e: 535.6 (M+H)⁺.

### EXAMPLE 126

N-(3-{1-[(3S)-3-(3-Acetylphenoxy)-3-Phenylpropyl]-4-Piperidinyl}Phenyl)-2-Methylpropanamide

A mixture of N-(3-{1-[(3R)-3-hydroxy-3-phenylpropyl]-4-piperidinyl}phenyl)-2-methylpropanamide (5.20 mg, 0.0137 mmol), 2-acetylphenol (100 mg), triphenylphosphine (30.0 mg, 0.115 mmol) and diethyl azodicarboxylate (7.42 mg, 0.0426 mmol) in THF (0.50 mL) was stirred at room temperature for 3 days. Chromatography using silica preparative TLC plates [2.5% of NH₃ (2.0 M in methanol) in CHCl₃] gave the desired product (1.50 mg, 22.0% yield) as a thick oil: ¹H NMR (400 MHz, CDCl₃) δ 7.65 (m, 1H), 7.55 (s, 1H), 7.30-7.20 (m, 5H), 7.20 (m, 3H), 6.97 (m, 2H), 6.76 (apparent d, 1H), 5.49 (apparent dd, 1H, J=4.3, 8.0 Hz), 3.38 (m, 2H), 2.93 (m, 2H), 2.75 (s, 3H), 2.53 (apparent sept, partially hidden, 2H, J=7.6 Hz), 2.30-2.10 (m, 6H), 1.92 (m, 2H), 1.25 (d, 6H, J=6.9 Hz); ESMS m/e: 498.81 (M+), 499.6 (M+H)⁺.

### EXAMPLE 127

N-(3-{1-[(3S)-3-(2-Fluorophenoxy)-3-Phenylpropyl]-4-Piperidinyl}Phenyl)-2-Methylpropanamide

A mixture of N-(3-{1-[(3R)-3-hydroxy-3-phenylpropyl]-4-piperidinyl}phenyl)-2-methylpropanamide (5.20 mg, 0.0137 mmol), 2-fluorophenol (100 mg), triphenylphosphine (30.0 mg, 0.115 mmol) and diethyl azodicarboxylate (7.42 mg, 0.0426 mmol) in THF (0.50 mL) was stirred at room temperature for 3 days. Chromatography using silica preparative TLC plates [2.5% of NH₃ (2.0 M in methanol) in CHCl₃] gave the desired product (3.5 mg, 53.9% yield) as a thick oil: ¹H NMR (400 MHz, CDCl₃) δ 8.07 (s, 1H), 7.65 (m, 1H), 7.41 (s, 1H), 7.40-7.10 (m, 5H), 7.05 (m, 2H), 6.97 (apparent d, 1H, J=8.7 Hz), 6.86 (m, 2H), 6.79 (apparent dt, 1H, J=2.4, 7.9 Hz), 5.31 (apparent dd, 1H, J=4.5, 8.0 Hz), 3.39 (m, 2H), 2.97 (m, 2H), 2.53 (apparent sept, partially hidden, 2H, J=7.5 Hz), 2.3-2.1 (m, 6H), 1.92 (m, 2H), 1.25 (d, 6H, J=6.7 Hz); ESMS m/e: 475.7 (M+H)⁺.

### EXAMPLE 128

(4S)-N-(3-{4-[3-(Acetylamino)Phenyl]-1-Piperidinyl}Propyl)-4-(3,5-Difluorophenyl)-2-oxo-1,3-Oxazolidine-3-Carboxamide

Method:

Into a 20 ml vial was added N1-{3-[1-(aminopropyl)-1,2,3,6-tetrahydro-4-pyridinyl]phenyl}acetamide (15 mg, 0.054 mmol), 4-(3,5-Difluorophenyl)-2-oxo-oxazolidine-3-carboxylic acid-4-nitro-phenyl ester (39.3 mg, 1.08 mmol, 2 eq) and dichloromethane with 0.6% of Methanol (3 ml) at room temperature. After stirring at room temperature for 3 hrs, the reaction mixture was filtered, and purified by preparative silica TLC (19:1=chloroform:methanol) to afford the desired product (18.3 mg, 68% yield); ¹H NMR (400 MHz, CDCl₃) δ 8.09 (br s, 1H), 7.40 (d, 1H, J=8.0 Hz), 7.36-7.28 (m, 2H), 7.24 (t, 1H, J=8.0 Hz), 6.99 (d, 1H, J=8.0 Hz), 6.86-6.82 (m, 2H), 5.41 (dd, 1H, J=4.1, 9.0 Hz), 4.72 (t, 1H, J=9.0 Hz), 4.22 (dd, 1H, J=3.9, 9.1 Hz), 3.42-3.29 (m, 2H), 3.02 (d, 2H J=11.1 Hz), 2.52-2.38 (m, 3H), 2.16 (s, 3H), 2.08-1.98 (m, 2H), 1.86-1.70 (m, 6H); ESMS m/e: 501.2 (M+H)⁺; Anal. Calc. for C₂₆H₃₀F₂N₄O_{4+0.5}H₂O: C, 60.64; H, 6.18; N, 10.88. Found: C, 60.67; H, 5.79; N, 10.86.

### EXAMPLE 129

The synthetic method is the same as described for the synthesis of (4S)-N-(3-{4-[3-(acetylamino)phenyl]-1-piperidinyl}propyl)-4-(3,5-difluorophenyl)-2-oxo-1,3-oxazolidine-3-carboxamide.

(4S)-N-(3-{4-[3-(Acetylamino)Phenyl]-1-Piperidinyl}Propyl)-2-oxo-4-(3,4,5-Trifluorophenyl)-1,3-Oxazolidine-3-Carboxamide:

18.8 mg (67% yield); ¹H NMR (400 MHz, CDCl₃) δ 8.09 (br s, 1H), 7.41-7.20 (m, 3H), 7.02-6.91 (m, 3H), 5.37 (dd, 1H, J=3.8, 8.9 Hz), 4.71 (t, 1H, J=9 Hz), 4.21 (dd, 1H, J=4, 9.3 Hz), 3.43-3.27 (m, 2H), 3.02 (d, 2H, J=11.0 Hz), 2.53-2.37 (m, 3H), 2.16 (s, 3H), 2.08-1.97 (m, 2H), 1.85-1.69 (m, 6H); ESMS m/e: 519.2 (M+H)⁺; Anal. Calc. for C₂₆H₂₉F₃N₄O₄+0.5H₂O: C, 59.20; H, 5.73; N, 10.62. Found: C, 59.40; H, 5.35; N, 10.65.

### EXAMPLE 130

The synthetic method is the same as described for the synthesis of (4S)-N-(3-{4-[3-(acetylamino)phenyl]-1-piperidinyl}propyl)-4-(3,5-difluorophenyl)-2-oxo-1,3-oxazolidine-3-carboxamide.

N-(3-{4-[3-(Acetylamino)Phenyl]-1-Piperidinyl}Propyl)-4-(3,4-Difluorophenyl)-5,5-Dimethyl-2-oxo-1,3-Oxazolidine-3-Carboxamide: 19.6 mg (68% yield); ¹H NMR (400 MHz, CDCl₃) δ 8.18 (t, 1H, J=5.9 Hz), 7.41 (d, 1H, J=8.8 Hz), 7.33 (s, 1H), 7.27-7.14 (m, 2H), 7.02-6.88 (m, 3H), 5.04 (s, 1H), 3.34 (qm, 2H, J=6.3 Hz), 3.02 (dm, 2H, J=10.9 Hz), 2.53-2.38 (m, 3H), 2.16 (s, 3H), 2.07-1.96 (m, 2H), 1.87-1.69 (m, 6H), 1.62 (s, 3H), 1.02 (s, 3H); ESMS m/e: 529.3 (M+H)⁺; Anal. Calc. for C₂₈H₃₄F₂N₄O₄: C, 63.62; H, 6.48; N, 10.60. Found: C, 63.15; H, 6.27; N, 10.48.

### EXAMPLE 131

The synthetic method is the same as described for the synthesis of (4S)-N-(3-{4-[3-(acetylamino)phenyl]-1-piperidinyl}propyl)-4-(3,5-difluorophenyl)-2-oxo-1,3-oxazolidine-3-carboxamide.

(4S,SR)-N-(3-{4-[3-(Acetylamino)Phenyl]-1-Piperidinyl}Propyl)-4-(3,4-Difluorophenyl)-5-Methyl-2-oxo-1,3-Oxazolidine-3-Carboxamide: 20.5 mg (74% yield); ¹H NMR (400 MHz, CDCl₃) δ 8.14 (t, 1H, J=5.5 Hz), 7.40 (d, 1H, J=7.8 Hz), 7.37-6.89 (m, 6H), 5.35 (d, 1H, J=7.5 Hz), 5.02-4.93 (m, 1H), 3.41-3.25 (m, 2H), 3.02 (d, 2H, J=10.8 Hz), 2.53-2.37 (m, 3H), 2.16 (s, 3H), 2.07 (m, 2H), 1.89-1.68 (m, 6H), 1.04 (d, 3H, J=6.4 Hz); ESMS m/e: 515.3 (M+H)⁺; Anal. Calc. for C₂₇H₃₂F₂N₄O₄+0.5H₂O: C, 61.94; H, 6.35; N, 10.70. Found: C, 61.90; H, 6.13; N, 10.64.

### EXAMPLE 132

The synthetic method is the same as described for the synthesis of (4S)-N-(3-{4-[3-(acetylamino)phenyl]-1-piperidinyl}propyl)-4-(3,5-difluorophenyl)-2-oxo-1,3-oxazolidine-3-carboxamide.

N-(3-{4-[3-(Acetylamino)Phenyl]-1-Piperidinyl}Propyl)-4-(4-Fluorobenzyl)-2-oxo-1,3-Oxazolidine-3-Carboxamide:

17.4 mg (65% yield); ¹H NMR (400 MHz, CDCl₃) δ 8.08 (t, 1H, J=5.6 Hz), 7.4 (d, 1H, J=7.2 Hz), 7.34 (s, 1H), 7.28-7.14 (m, 3H), 7.05-6.95 (m, 3H), 4.69-4.60 (m, 1H), 4.26 (t, 1H, J=8.8 Hz), 4.15 (dd, 1H, J=3.2, 9 Hz), 3.43 (q, 2H, J=6.2 Hz), 3.3 (dm 1H, J=13.6 Hz), 3.04 (dm, 2H, J=11 Hz), 2.87 (dd, 1H, J=9.3, 14.4 Hz), 2.53-2.42 (m, 3H), 2.16 (s, 3H), 2.09-1.99 (m, 2H), 1.87-1.65 (m, 6H); ESMS m/e: 497.3 (M+H)⁺; Anal. Calc. for C₂₇H₃₃FN₄O₄+0.5H₂O: C, 64.14; H, 6.78; N, 11.08. Found: C, 64.26; H, 6.39; N, 11.12.

### EXAMPLE 133

2-Methyl-N-(3-{1-[(3R)-3-(2-Nitrophenoxy)-3-Phenylpropyl]-4-Piperidinyl}Phenyl)Propanamide

A mixture of N-(3-{1-[(3S)-3-hydroxy-3-phenylpropyl]-4-piperidinyl}phenyl)-2-methylpropanamide (5.20 mg, 0.0137 mmol), 2-nitrophenol (100 mg), triphenylphosphine (30.0 mg, 0.115 mmol) and diethyl azodicarboxylate (7.42 mg, 0.0426 mmol) in THF (0.50 mL) was stirred at room temperature for 3 days. Chromatography using silica preparative TLC plates [2.5% of NH₃ (2.0 M in methanol) in CHCl₃] gave the desired product (2.37 mg, 34.5% yield) as a thick oil: ¹H NMR (400 MHz, CDCl₃) δ 7.84 (d, 1H), 7.90 (m, 1H), 7.45 (m 1H), 7.30-7.20 (m, 5H), 7.20 (m, 2H), 6.98 (m, 2H), 6.89 (apparent d, 1H, J=7.7 Hz), 5.62 (apparent dd, 1H, J=4.1, 8.9 Hz), 3.10 (m, 2H), 2.60 (m, 2H), 2.53 (m, 2H), 2.30-2.10 (m, 6H), 1.90 (m, 2H), 1.25 (d, 6H, overlapped); ESMS m/e: 502.3 (M+H)⁺.

### EXAMPLE 134

N-(3-{1-[(3S)-3-([1,1′-Biphenyl]-4-yloxy)-3-Phenylpropyl]-4-Piperidinyl)Phenyl)-2-Methylpropanamide

A mixture of N-(3-{1-[(3R)-3-hydroxy-3-phenylpropyl]-4-piperidinyl}phenyl)-2-methylpropanamide (5.20 mg, 0.0137 mmol), 4-phenylphenol (100 mg), triphenylphosphine (30.0 mg, 0.115 mmol) and diethyl azodicarboxylate (7.42 mg, 0.0426 mmol) in THF (0.50 mL) was stirred at room temperature for 3 days. Chromatography using silica preparative TLC plates [2.5% of NH₃ (2.0 M in methanol) in CHCl₃] gave the desired product (3.00 mg, 41.2% yield) as a thick oil: ¹H NMR (400 MHz, CDCl₃) δ 8.06 (s, 1H), 7.48 (m, 2H), 7.40-7.30 (m, 8H), 7.30-7.25 (m, 4H), 6.97 (apparent d, 1H, J=7.6 Hz), 6.91 (apparent d, 2H, J=8.7 Hz), 5.34 (apparent dd, 1H, J=4.4, 8.0 Hz), 3.40 (m, 2H), 2.98 (m, 2H), 2.53 (apparent sept, partially hidden, 1H, J=8.1 Hz), 2.44 (m, 1H), 2.30-2.10 (m, 6H), 1.93 (d, 2H), 1.26 (d, 6H, J=6.9 Hz); ESMS m/e: 533.4 (M+H)⁺.

### EXAMPLE 135

2-Methyl-N-(3-{1-[(3R)-3-(3-Nitrophenoxy)-3-Phenylpropyl]-4-Piperidinyl}Phenyl)Propanamide

A mixture of N-(3-{1-[(3S)-3-hydroxy-3-phenylpropyl]-4-piperidinyl}phenyl)-2-methylpropanamide (5.20 mg, 0.0137 mmol), 3-nitrophenol (100 mg), triphenylphosphine (30.0 mg, 0.115 mmol) and diethyl azodicarboxylate (7.42 mg, 0.0426 mmol) in THF (0.50 mL) was stirred at room temperature for 3 days. Chromatography using silica preparative TLC plates [2.5% of NH₃ (2.0 M in methanol) in CHCl₃] gave the desired product (2.80 mg, 40.8% yield) as a thick oil: ¹H NMR (400 MHz, CDCl₃) δ 7.76 (dm, 1H), 7.71 (t, 1H, J=1.8 Hz), 7.50-7.40 (m, 2H), 7.40-7.25 (m, 7H), 7.17 (apparent dd, 1H, J=2.4, 8.2), 6.97 (apparent d, 1H, J=7.7 Hz), 5.45 (apparent dd, 1H, J=5.0, 8.1 Hz), 3.45 (m, 2H), 2.89 (m, 2H), 2.53 (apparent sept, partially hidden, 2H, J=8.3 Hz), 2.30-2.10 (m, 6H), 1.92 (m, 2H), 1.25 (d, 6H, J=6.8 Hz); ESMS m/e: 502.3 (M+H)⁺.

### EXAMPLE 136

N-(3-{1-[(3S)-3-(2-Ethoxyphenoxy)-3-Phenylpropyl]-4-Piperidinyl}Phenyl)-2-Methylpropanamide

A mixture of N-(3-{1-[(3R)-3-hydroxy-3-phenylpropyl]-4-piperidinyl}phenyl)-2-methylpropanamide (5.20 mg, 0.0137 mmol), 2-ethoxyphenol (100 mg), triphenylphosphine (30.0 mg, 0.115 mmol) and diethyl azodicarboxylate (7.42 mg, 0.0426 mmol) in THF (0.50 mL) was stirred at room temperature for 3 days. Chromatography using silica preparative TLC plates [2.5% of NH₃ (2.0 M in methanol) in CHCl₃] gave the desired product (1.16 mg, 15.5% yield) as a thick oil: ¹H NMR (400 MHz, CDCl₃) δ 8.06 (s, 1H), 7.52 (s, 1H), 7.40-7.33 (m, 4H), 7.30-7.20 (m, 3H), 6.97 (apparent d, 1H, J=7.7 Hz), 6.88 (m, 2H), 6.68 (m, 2H), 5.21 (m, 1H), 4.11 (q, 2H, J=7.3 Hz), 3.37 (m, 2H), 2.71 (m, 2H), 2.53 (apparent sept, partially hidden, 2H, J=7.6 Hz), 2.30-2.10 (m, 6H), 1.89 (m, 2H), 1.49 (t, 3H, J=7.3 Hz), 1.25 (d, 6H, J=6.8 Hz); ESMS m/e: 501.4 (M+H)⁺.

### EXAMPLE 137

2-Methyl-N-(3-{1-[(3S)-3-(1-Naphthyloxy)-3-Phenylpropyl]-4-Piperidinyl}Phenyl)Propanamide

A mixture of N-(3-{1-[(3R)-3-hydroxy-3-phenylpropyl]-4-piperidinyl}phenyl)-2-methylpropanamide (5.20 mg, 0.0137 mmol), 1-naphthol (100 mg), triphenylphosphine (30.0 mg, 0.115 mmol) and diethyl azodicarboxylate (7.42 mg, 0.0426 mmol) in THF (0.50 mL) was stirred at room temperature for 3 days. Chromatography using silica preparative TLC plates [2.5% of NH₃ (2.0 M in methanol) in CHCl₃] gave the desired product (4.30 mg, 66.2% yield) as a thick oil: ¹H NMR (400 MHz, CDCl₃) δ 8.06 (s, 1H), 7.72 (d, 1H, J=8.5 Hz), 7.59 (d, 1H, J=8.5 Hz), 7.5 (m, 2H), 7.45-7.30 (m, 6H), 7.25 (m, 3H), 7.17 (apparent dd, 1H, J=2.6, 9.0 Hz), 7.01 (apparent d, 1H, J=2.6 Hz), 6.97 (apparent d, 1H, J=7.9 Hz), 5.46 (apparent dd, 1H, J=4.5, 8.1 Hz), 3.12 (m, 2H), 2.61 (m, 2H), 2.53 (apparent sept, partially hidden, 2H, J=7.9 Hz), 2.30-2.10 (m, 6H), 1.90 (m, 2H), 1.25 (d, 6H, J=7.3 Hz, overlapped); ESMS m/e: 507.2 (M+H)⁺.

### EXAMPLE 138

N-(3-{1-[(3S)-3-(1,3-Dioxo-1,3-Dihydro-2H-Isoindol-2-yl)-3-Phenylpropyl]-4-Piperidinyl}Phenyl)-2-Methylpropanamide

Step 1:

2-[(1S)-3-Chloro-1-Phenylpropyl]-1H-Isoindole-1,3(2H)-Dione:

A mixture of phthalimide (0.147 g, 1.0 mmol), (R)-(+)-3-chloro-phenyl-1-propanol (0.171 g, 1.0 mmol), triphenylphosphine (0.262 g, 1.0 mmol), diethyl azodicarboxylate (0.174 g, 1.0 mmol) in 5.0 mL of THF was stirred at room temperature for 24 h. The reaction mixture was concentrated in vacuo. The residue was washed with pentane (×3) and the combined pentane extracts were concentrated and chromatographed (silica with hexanes-EtOAc 8:1 as the eluent) to give the desired product (as described as a general procedure by: Srebnik, M.; Ramachandran, P. V.; Brown, H. C. J. Org. Chem. 1988, 53, 2916-2920) afforded the desired product (0.121 g, 50.2%) as a yellow solid: ¹H NMR (400 MHz, CDCl₃) δ 7.82 (apparent dd, 2H, J=2.9 Hz), 7.70 (apparent dd, 2H, J=2.9 Hz), 7.56 (m, 2H), 7.39-7.27 (m, 3H), 5.64 (apparent dd, 1H, J=7.0, 9.2 Hz), 3.57 (m, 2H), 3.05 (m, 1H), 2.82 (apparent sept, 1H, J=7.0 Hz); ESMS m/e: 300.13 (M+H)⁺.

Step 2:

N-(3-{1-[(3S)-3-(1,3-Dioxo-1,3-Dihydro-2H-Isoindol-2-yl)-3-Phenylpropyl]-4-Piperidinyl}Phenyl)-2-Methylpropanamide:

A mixture of potassium carbonate (29.2 mg, 0.211 mmol), sodium iodide (47.5 mg, 0.317 mmol), 2-methyl-N-[3-(4-piperidinyl)phenyl]propanamide (51.8 mg, 0.211 mmol) 2-[(1S)-3-chloro-1-phenylpropyl]-1H-isoindole-1,3(2H)-dione

(63.1 mg, 0.211 mmol) in DMF (5.0 mL) was stirred at 100° C. for 3 hrs, at which time TLC indicated that the reaction was complete. The reaction mixture was poured into water (50 mL) and the aqueous layer was extracted with methylene chloride (3×30 mL). The combined organic extracts were washed with brine (30 mL), dried over MgSO₄ and concentrated under reduced pressure. The crude product was purified by Prep-TLC plates [2.5% of NH₃ (2.0 M in methanol) in CHCl₃]

to give the desired product (74.1 mg, 77.1%) as a thick oil: ¹H NMR (400 MHz, CDCl₃) δ 7.83 (apparent dd, 2H, J=2.9 Hz), 7.69 (apparent dd, 2H, J=2.9 Hz), 7.56 (apparent dt, 3H, J=2.9, 7.3 Hz), 7.33 (m, 4H), 7.21 (t, 1H, J=7.8 Hz), 7.09 (s, 1H), 6.81 (apparent d, 1H, J=7.8 Hz), 5.49 (apparent dd, 1H, J=5.5, 9.5 Hz), 2.98 (d, 1H, J=9.5 Hz), 2.87 (m, 2H), 2.50 (apparent sept, 1H, J=6.7 Hz), 2.40-2.35 (m, 4H), 1.94 (m, 2H), 1.70-1.50 (m, 4H), 1.25 (d, 6H, J=7.9 Hz); ESMS m/e: 510.37 (M+H)⁺.

### EXAMPLE 139

2-Methyl-N-(3-{1-[(3S)-3-(4-Phenoxyphenoxy)-3-Phenylpropyl]-4-Piperidinyl}Phenyl)Propanamide

Step 1:

4-{[(1S)-3-Chloro-1-Phenylpropyl]oxy}-(4-Phenoxy)Benzene:

A mixture of 4-phenoxyphenol (1.86 g, 10.0 mmol), (S)-(−)-3-chloro-phenyl-1-propanol (1.70 g, 10.0 mmol), triphenylphosphine (2.62 g, 10.0 mmol), diethyl azodicarboxylate (1.57 mL, 10.0 mmol) in 5.0 mL of THF was stirred at room temperature for 24 h. The reaction mixture was concentrated in vacuo. The residue was washed with pentane (×3) and the combined pentane extracts were concentrated and chromatographed (silica with hexanes-EtOAc 97:3 as the eluent) to give the desired product (as described as a general procedure by: Srebnik, M.; Ramachandran, P. V.; Brown, H. C. J. Org. Chem. 1988, 53, 2916-2920) afforded the desired product as a thick oil which solidified on standing (2.51 g, 75.7%): ¹H NMR (400 MHz, CDCl₃) δ 7.4-7.23 (m, 7H), 7.03 (apparent t, 1H, J=7.3 Hz), 6.91 (apparent dm, 2H, J=7.8 Hz), 6.93 (apparent q, 4H, J=7.8 Hz), 5.31 (apparent dd, 1H, J=4.5, 8.6 Hz), 3.82 (m, 1H), 3.62 (apparent quintet, 1H, J=5.6 Hz), 2.47 (m, 1H), 2.20 (m, 1H).

Step 2:

2-Methyl-N-(3-{1-[(3S)-3-(4-Phenoxyphenoxy)-3-Phenylpropyl]-4-Piperidinyl}Phenyl)Propanamide:

A mixture of 2-methyl-N-[3-(4-piperidinyl)phenyl] propanamide (65.5 mg, 0.266 mmol), 4-{[(1S)-3-chloro-1-phenylpropyl]oxy}-(4-phenoxy)benzene (0.100 mg, 0.296 mmol), potassium carbonate (40.9 mg, 0.296 mmol) and sodium iodide (67.0 mg, 0.444 mmol) in DMF (1.0 mL) at 100° C. for 3 hours. The reaction mixture was poured into water (50 mL) and the aqueous layer was extracted with methylene chloride (3×30 mL). The combined organic extracts were washed with brine (30 mL), dried over MgSO₄ and concentrated under reduced pressure. The crude product was purified by Prep-TLC plates [2.5% of NH₃ (2.0 M in methanol) in CHCl₃] to give the desired product (0.109 g, 74.6%) as a thick oil: ¹H NMR (400 MHz, CDCl₃) δ 7.48 (s, 1H), 7.40-7.30 (m, 4H), 7.20-7.10 (m, 6H), 7.09 (s, 1H), 6.99 (apparent d, 1H, J=7.8 Hz), 6.98 (apparent t, 1H, J=7.8 Hz), 6.93 (apparent d, 2H, J=8.4 Hz), 6.84 (m, 2H), 5.20 (apparent dd, 1H, J=4.4, 8.5 Hz), 3.03 (m, 2H), 2.51 (m, 4H), 2.24 (apparent sept, 1H, J=7.8 Hz), 2.20-2.10 (m, 3H), 1.90 (m, 4H), 1.25 (d, 6H, J=7.9 Hz); ESMS m/e: 549.41 (M+H)⁺; Anal. Calc. for C₃₆H₄₀N₂O₃: C, 78.80; H, 7.35; N, 5.11. Found: C, 78.58; H, 7.48; N, 5.09.

### EXAMPLE 140

N-(4-{1-[(3R)-3-(3,4-Dimethoxyphenoxy)-3-Phenylpropyl]-4-Piperidinyl}Phenyl)-2-Methylpropanamide

Step 1:

1-[(3R)-3-(3,4-DIMETHOXYPHENOXY)-3-PHENYLPROPYL]-4-(4-NITROPHENYL)-1,2,3,6-TETRAHYDROPYRIDINE:

A mixture of potassium carbonate (24.0 mg, 0.174 mmol), sodium iodide (39.0 mg, 0.260 mmol), 4-(4-nitrophenyl)-1,2,3,6-tetrahydropyridine (35.4 mg, 0.174 mmol) and 4-{[(1R)-3-chloro-1-phenylpropyl]oxy}-1,2-dimethoxybenzene (53.4 mg, 0.174 mmol) in DMF (0.5 mL) was stirred at 100° C. for 3 hrs, at which time TLC indicated that the reaction was complete. The reaction mixture was poured into water (5.0 mL) and the aqueous layer was extracted with methylene chloride (3×30 mL). The combined organic extracts were washed with brine (30 mL), dried over MgSO₄ and concentrated under reduced pressure. The crude product was purified by Prep-TLC plates [1:1=hexane:ethyl acetate with 1% NH₃] afforded the product (63.1 mg, 76.6%) as a yellow oil. The product was used in next reaction without further purification.

Step 2:

4-{1-[(3R)-3-(3,4-Dimethoxyphenoxy)-3-Phenylpropyl]-4-Piperidinyl}Aniline:

A 25-mL RB flask, equipped with a hydrogen-filled balloon, was charged with 1-[(3R)-3-(3,4-dimethoxyphenoxy)-3-phenylpropyl]-4-(4-nitrophenyl)-1,2,3,6-tetrahydropyridine (63.0 mg, 0.133 mmol), Palladium on Carbon (5.0 mol-eq%, 0.00665 mmol, 7.04 mg) and ethanol (2.0 mL) at room temperature. After 1 hr the reaction mixture was filtered through a plug of Celite 545 and concentrated under reduced pressure. The crude product (54.1 mg, 89.4%) was used in next reaction without further purification.

Step 3:

N-(4-{1-[(3R)-3-(3,4-Dimethoxyphenoxy)-3-Phenylpropyl]-4-Piperidinyl}Phenyl)-2-Methylpropanamide:

A mixture of 4-{1-[(3R)-3-(3,4-dimethoxyphenoxy)-3-phenylpropyl]-4-piperidinyl}aniline (5.31 mg, 0.0119 mmol), isobutyryl chloride (2.08 mg, 0.019 mmol), N,N-diisopropylethylamine (8.40 mg, 0.0650 mmol) in methylene chloride (1.0 mL) was stirred at room temperature for 24 hours. The reaction mixture was concentrated and chromatographed using a preparative TLC plate [2.5% of NH₃ (2.0 M in methanol) in CHCl₃] to give the product (3.5 mg, 56.5%) as a thick oil: ¹H NMR (400 MHz, CDCl₃) δ 7.38 (d, 1H, J=8.6 Hz), 7.30-7.20 (m, 4H), 7.20(m, 1H), 7.11 (d, 2H, J=8.6 Hz), 7.04 (s, 1H), 6.57 (d, 1H, J=8.3 Hz), 6.44 (d, 1H, J=2.6 Hz), 6.22 (dd, 1H, J=2.6, 8.3 Hz), 5.09 (apparent dd, 1H, J=4.4, 8.1 Hz), 3.72 (s, 3H), 3.70 (s, 3H), 3.08 (m, 2H), 2.57 (m, 2H), 2.43 (apparent sept, partially hidden, 2H, J=6.8 Hz), 2.30-2.10 (m, 6H), 1.80 (m, 2H), 1.25 (d, 6H, J=7.9 Hz); ESMS m/e: 517.3 (M+H)⁺.

### EXAMPLE 141

N-(3-{1-[(3S)-3-(3-Acetylphenoxy)-3-Phenylpropyl]-4-Piperidinyl}Phenyl)-2-Methylpropanamide

Into a 25-mL RB-flask was added triphenylphosphine (9.80 mg, 0.0375 mmol), diethyl azodicarboxylate (5.22 mg, 0.0300 mmol), N-(3-{1-[(3R)-3-hydroxy-3-phenylpropyl]-4-piperidinyl}phenyl)-2-methylpropanamide (9.53 mg, 0.0250 mmol), 3-hydroxyacetophenone (100 mg) and THF (1.0 mL) at room temperature. The reaction mixture was stirred at room temperature overnight (16 hrs). The solvent was removed under reduced pressure and the residue was purified by preparative TLC plates [2.5% of NH₃ (2.0 M in methanol) in CHCl₃] to afford the desired product (2.73 mg, 39.9%) as a thick oil: ¹H NMR δ 7.70-7.64 (m, 2H), 7.54 (m, 2H), 7.49-7.44 (m, 6H), 7.25 (m, 1H), 7.05 (d, 1H, J=8.3 Hz), 6.96 (apparent d, 1H, J=7.7 Hz), 5.34 (apparent dd, 1H, J=4.8, 8.2 Hz), 3.15 (m, 2H), 2.67 (m, 2H), 2.52 (s, 3H), 2.53 (apparent sept, partially hidden, 2H, J=7.6 Hz), 2.30-2.10 (m, 6H), 1.89 (m, 2H), 1.25 (d, 6H, J=6.9 Hz); ESMS m/e: 499.4 (M+H)⁺.

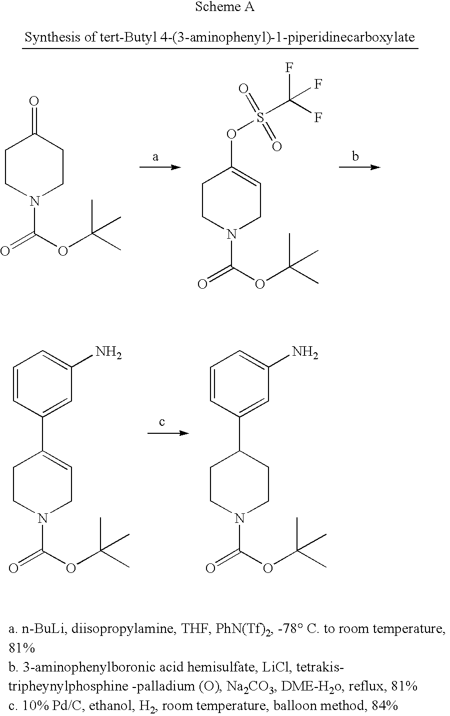

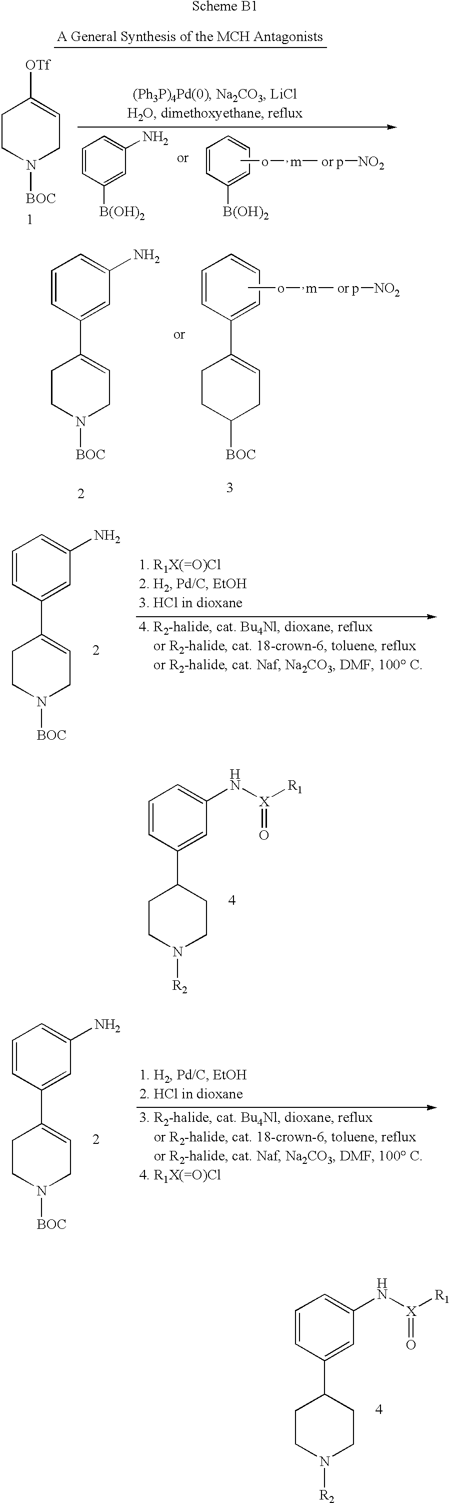

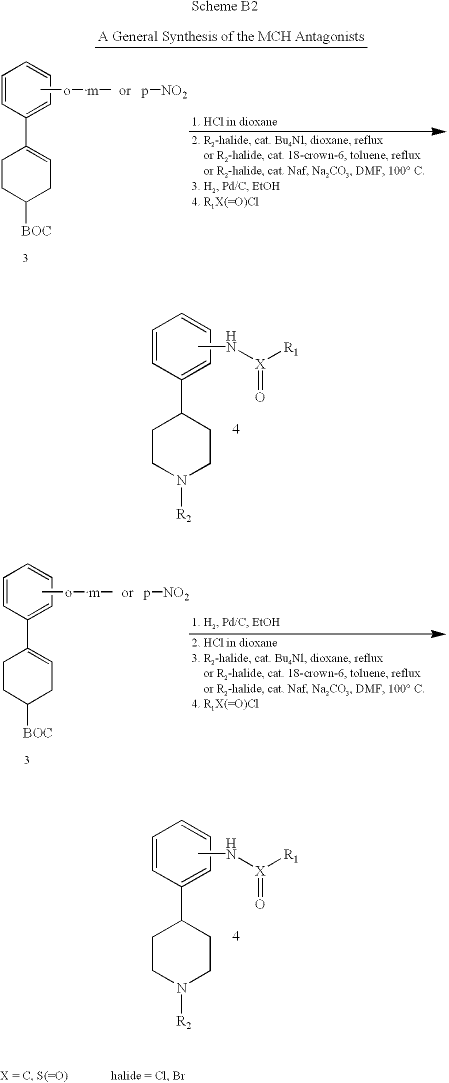

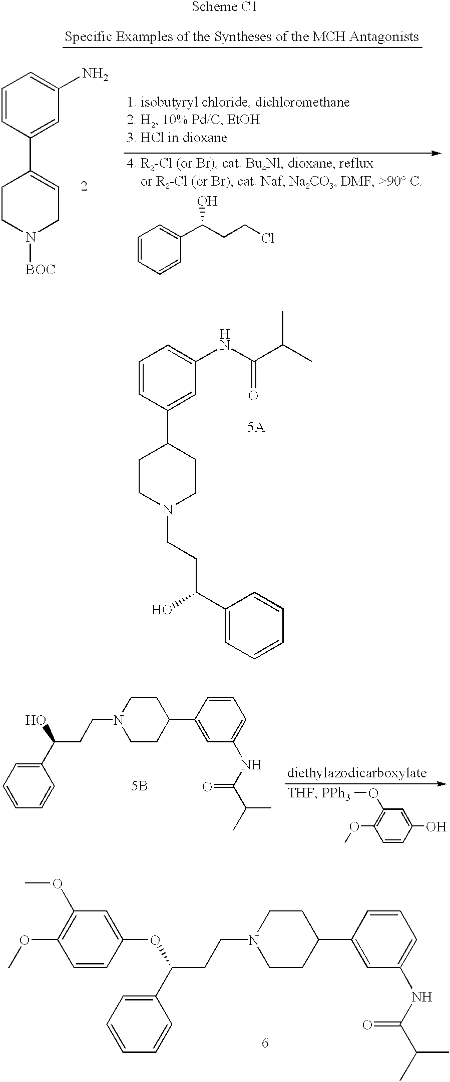

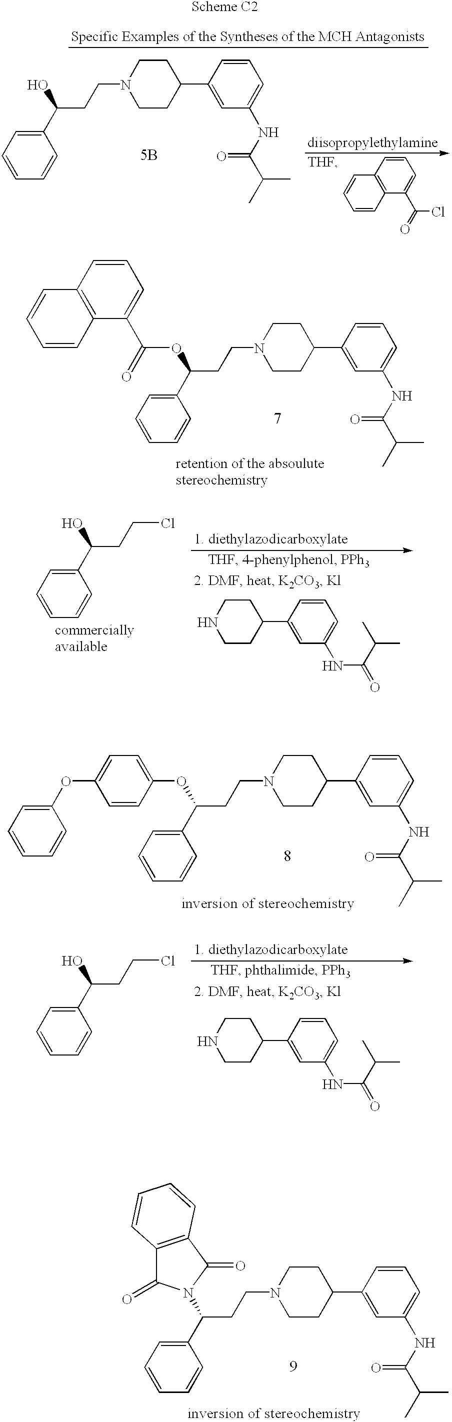

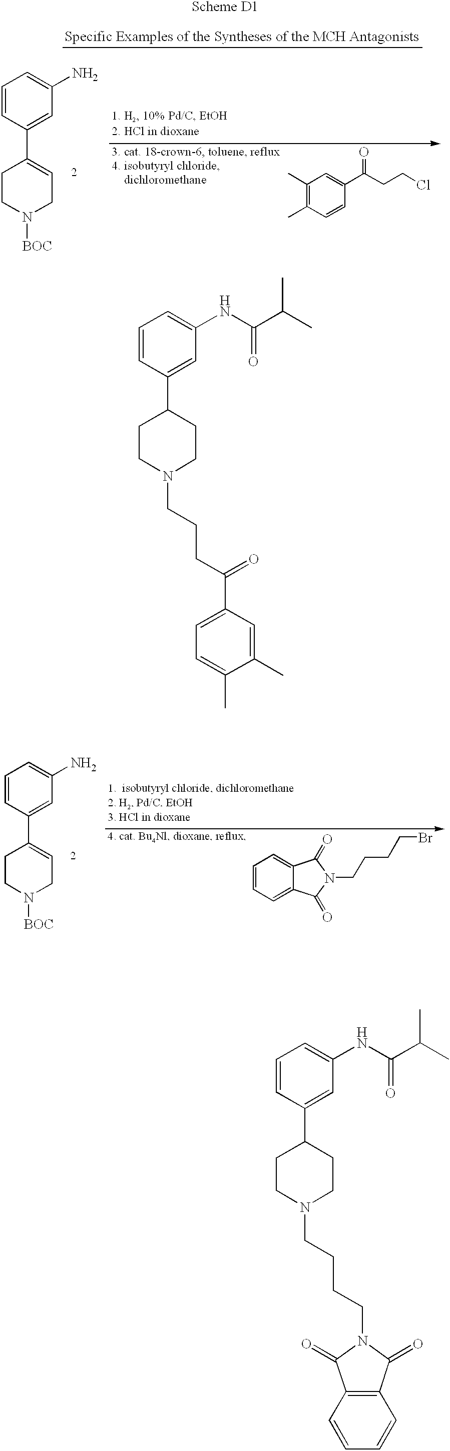

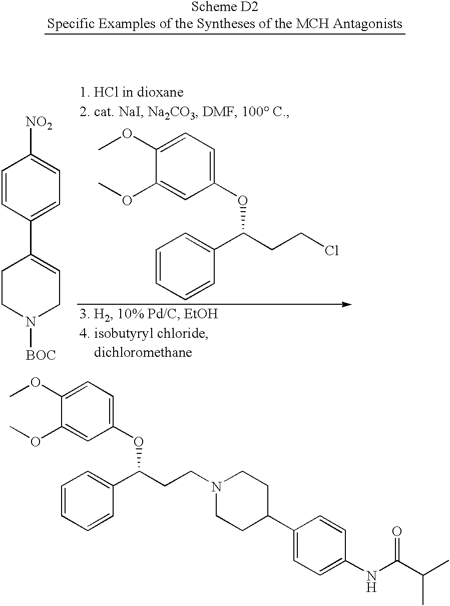

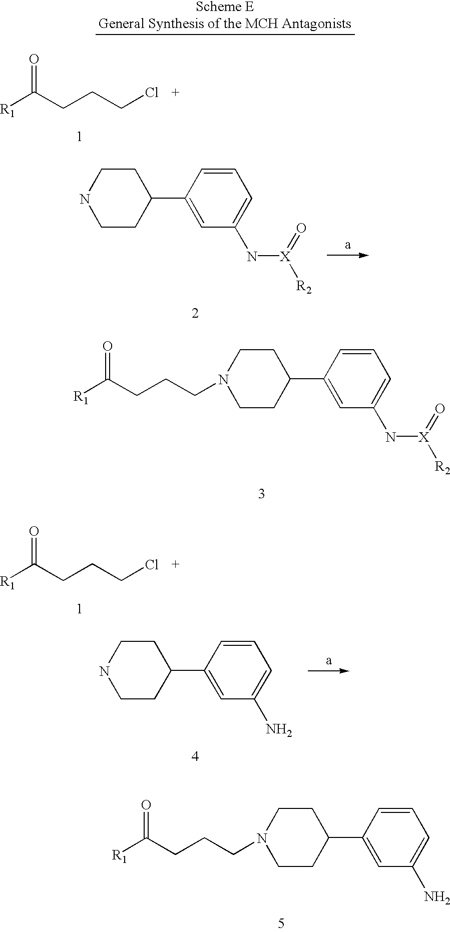

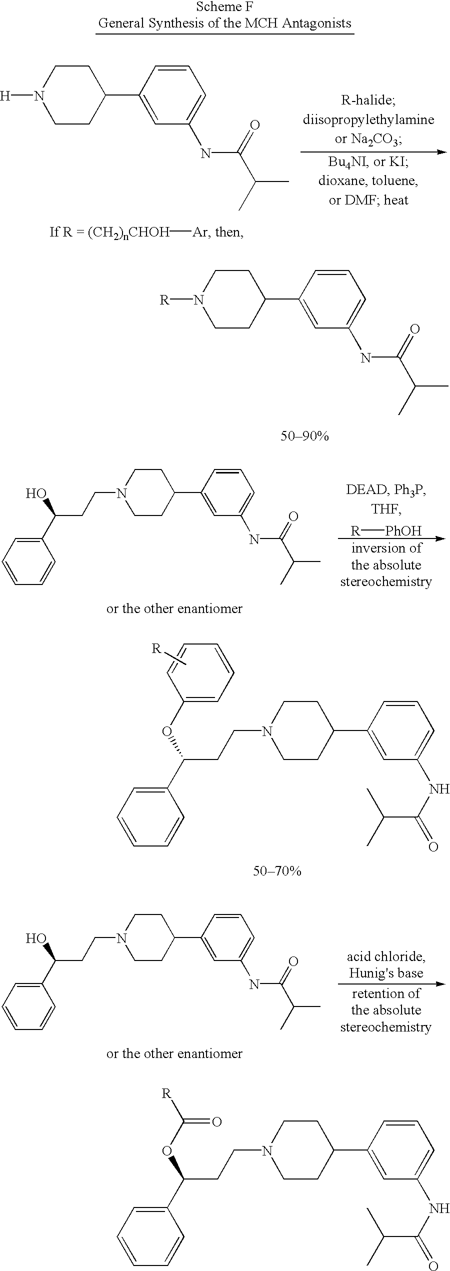

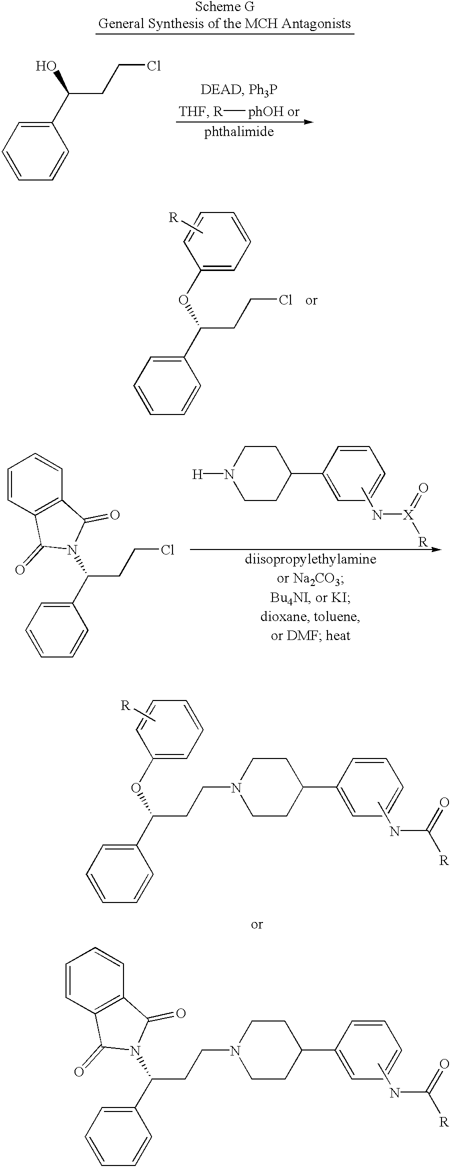

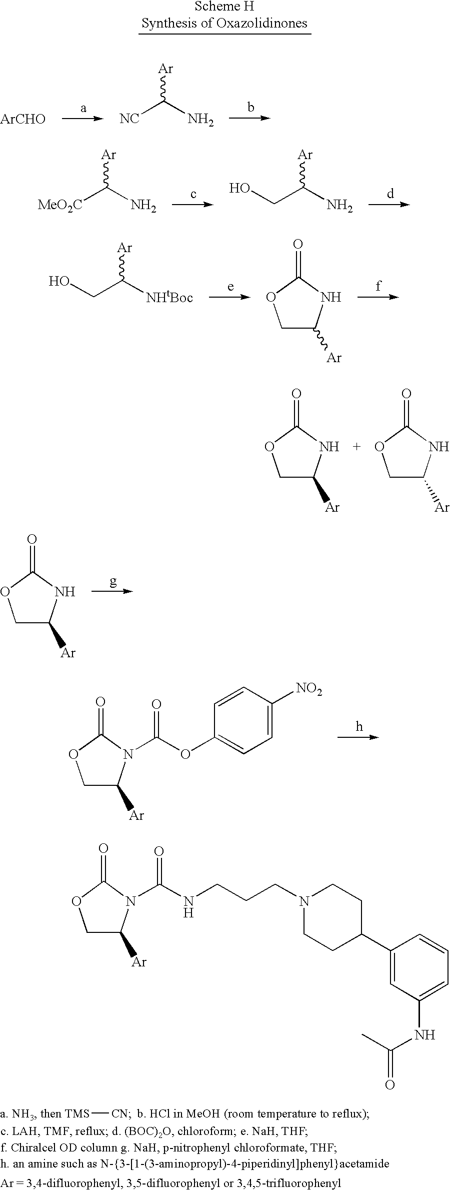

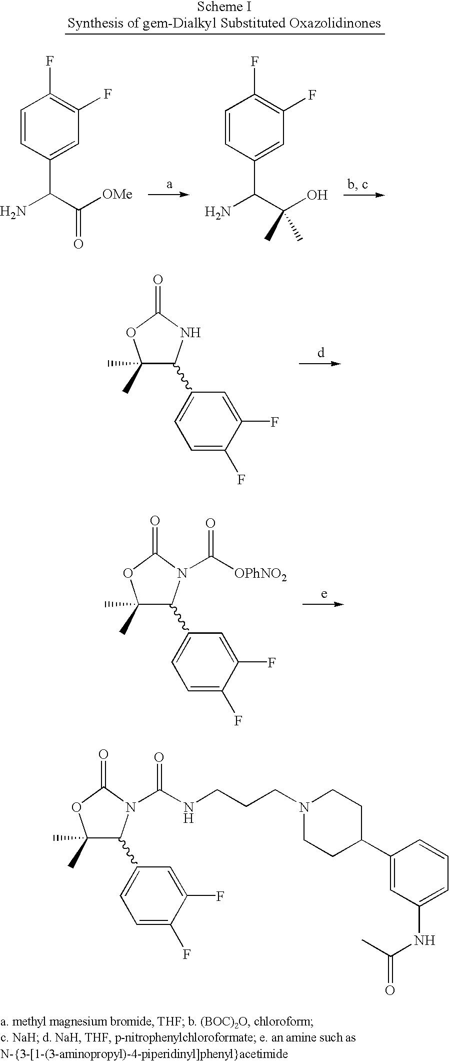

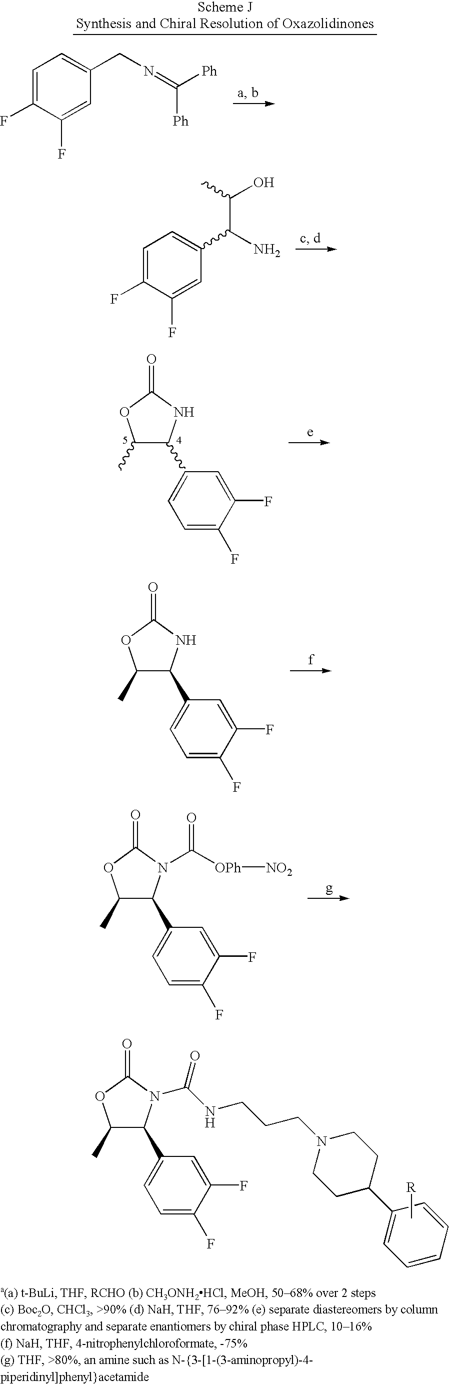

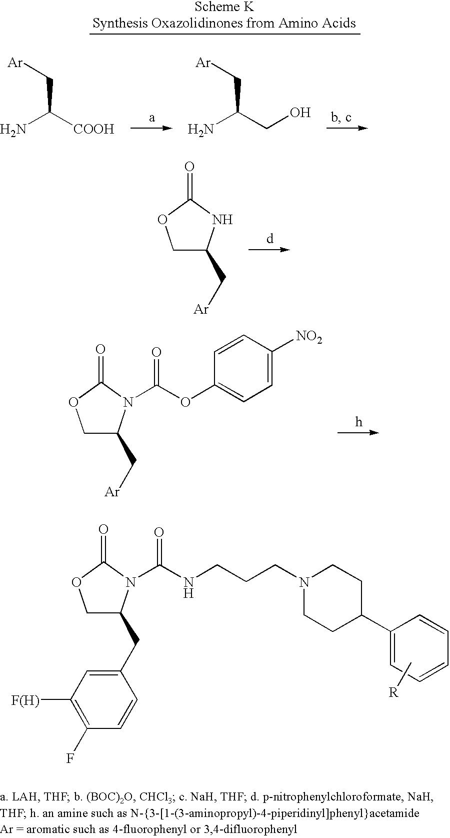

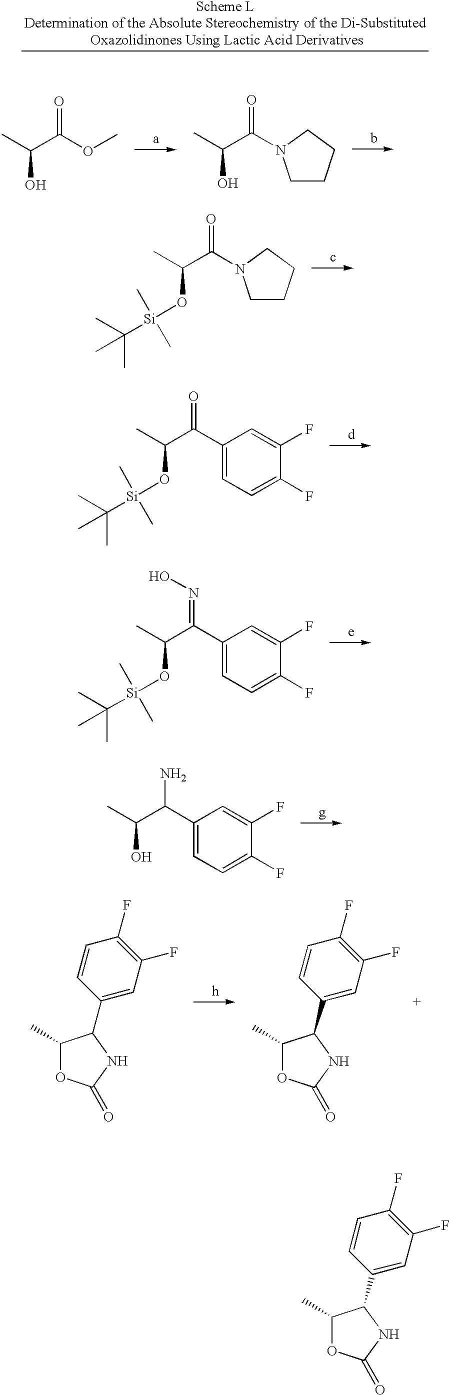

TABLE 1

| | | Ki (nM) |
|---|---|---|
| EXAMPLE No. | STRUCTURE | rMCH1 |
| 38 | 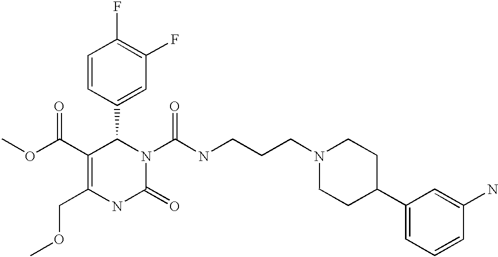 | 1.34 |
| 39 | 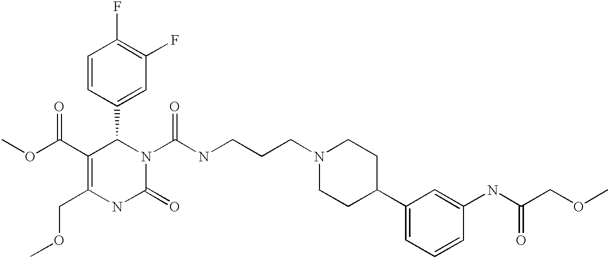 | 3.33 |
| 40 | 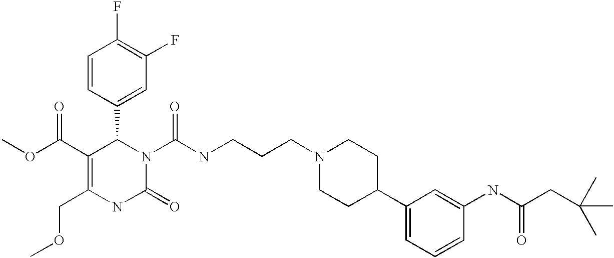 | 2.72 |
| 41 | 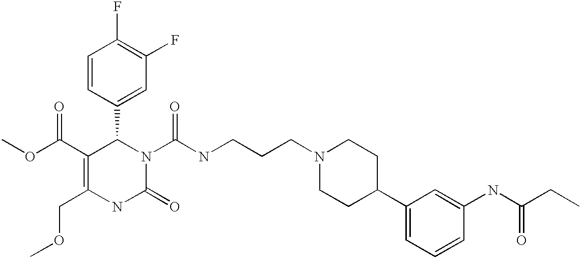 | 0.04 |
| 42 | 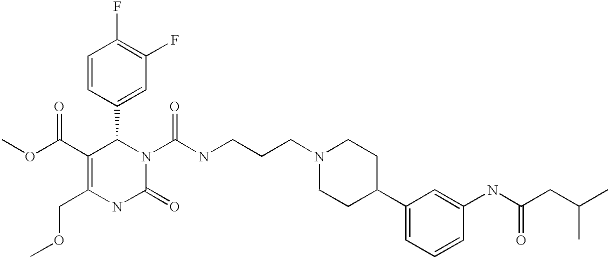 | 0.6 |
| 43 | 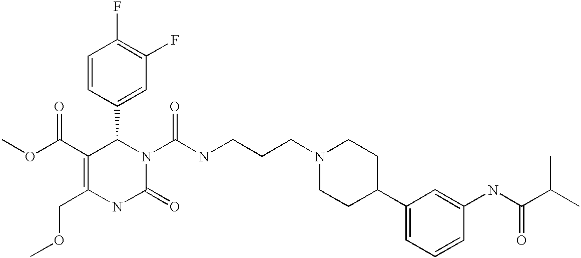 | 0.23 |
| 44 | 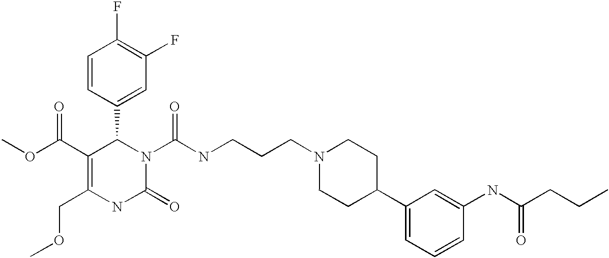 | 0.09 |
| 45 | 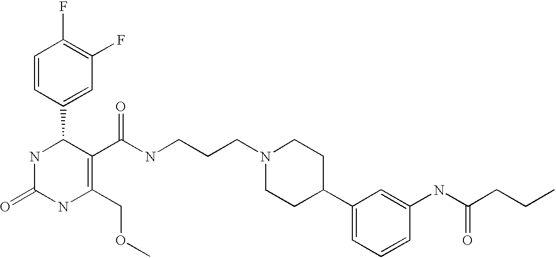 | 14.69 |
| 46 | 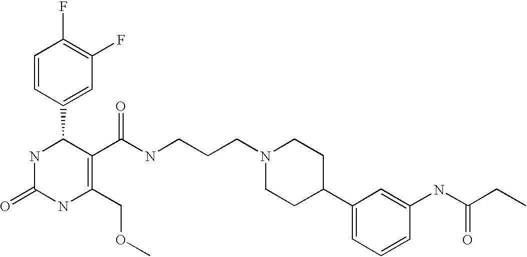 | 8.16 |
| 47 | 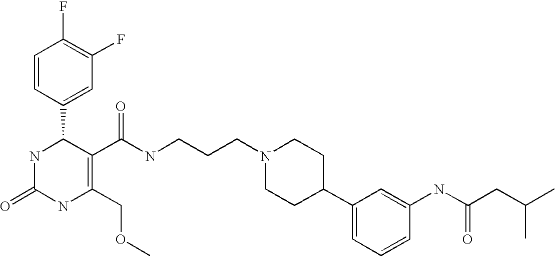 | 34.28 |
| 48 | 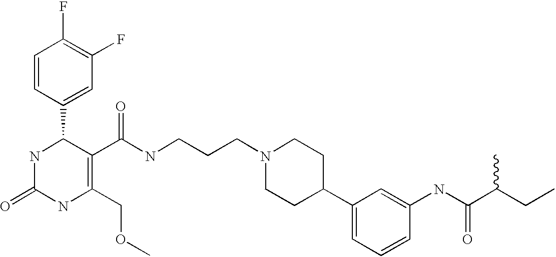 | 22.15 |
| 49 | 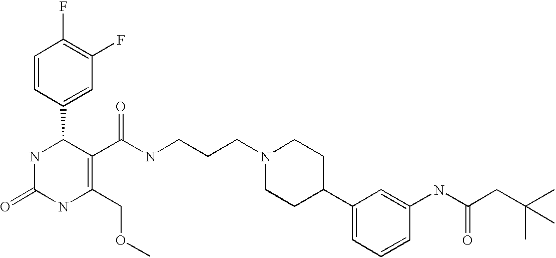 | 225.47 |
| 50 | 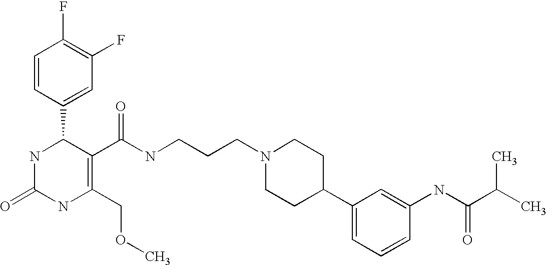 | 13.74 |
| 51 | 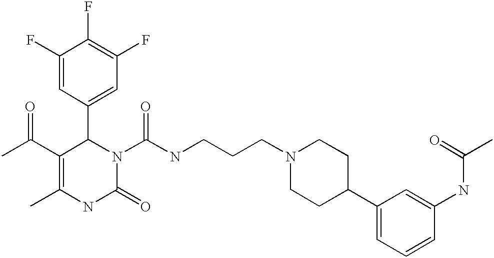 | 0.79 |
| 52 | 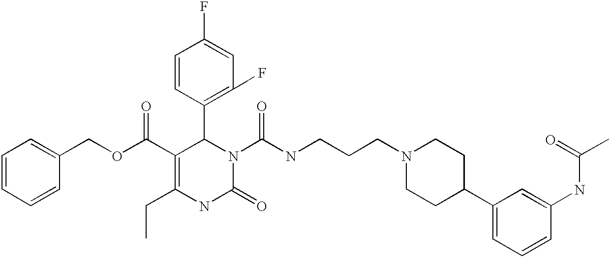 | 0.81 |
| 53 | 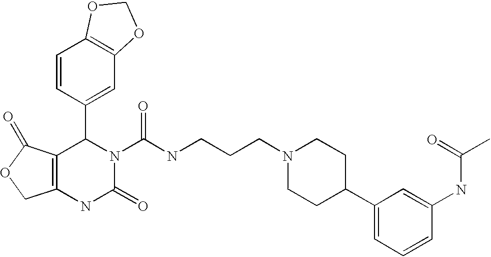 | 50.76 |
| 54 | 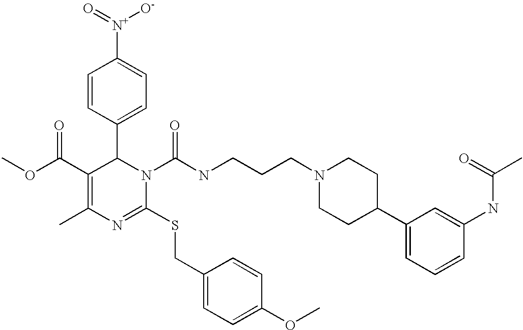 | 29.87 |
| 55 | 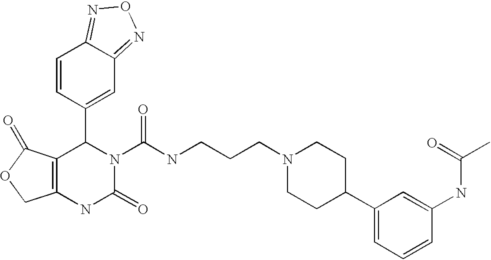 | 203.74 |
| 56 | 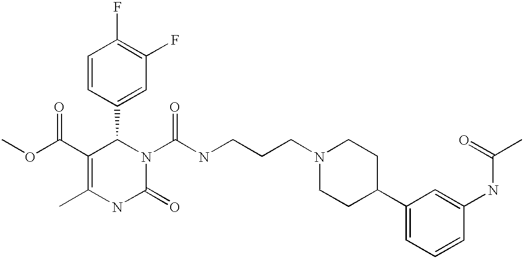 | 0.26 |
| 57 | 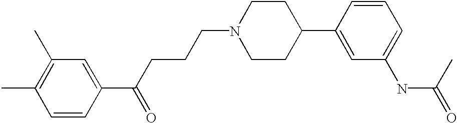 | 90 |
| 58 | 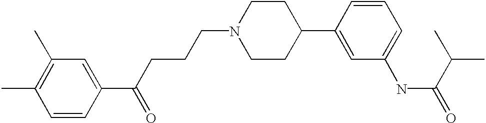 | 3.9 |
| 59 | 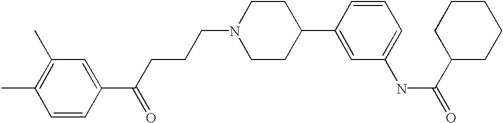 | 768 |
| 60 | 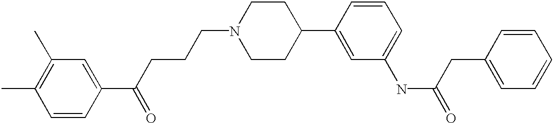 | 357 |
| 61 | 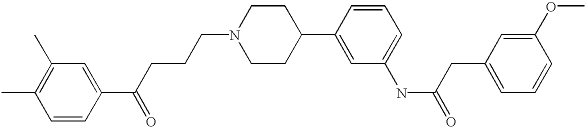 | 14.2 |
| 62 | 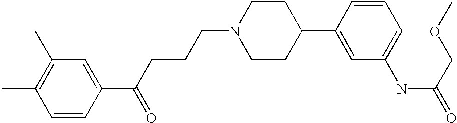 | 274 |
| 63 | 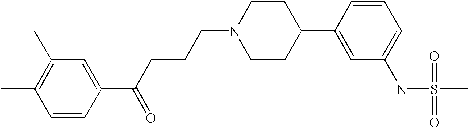 | 1000 |
| 64 | 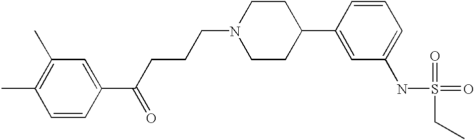 | 627 |
| 65 | 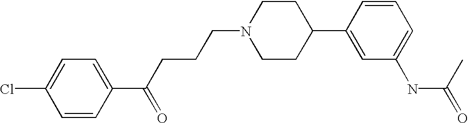 | 69 |
| 66 | 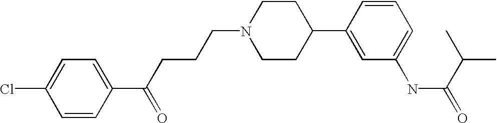 | 2.8 |
| 67 | 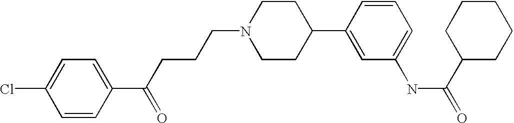 | 197 |
| 68 | 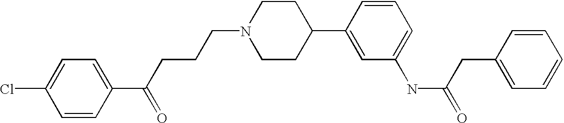 | 84 |
| 69 | 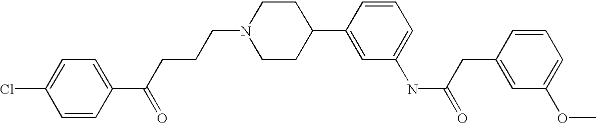 | 11.9 |
| 70 | 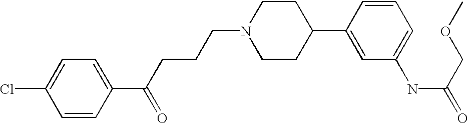 | 167 |
| 71 | 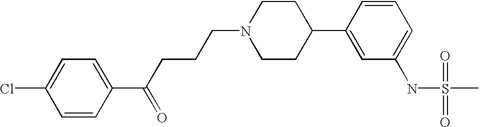 | 720 |
| 72 | 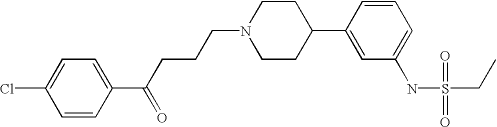 | 272 |
| 73 | 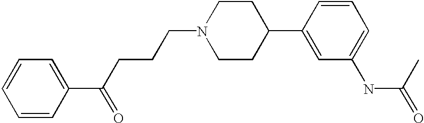 | 342 |
| 74 | 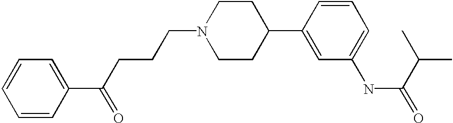 | 29.5 |
| 75 | 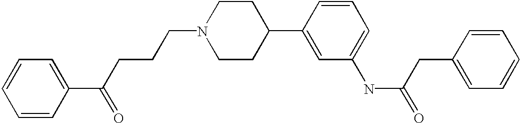 | 506 |
| 76 | 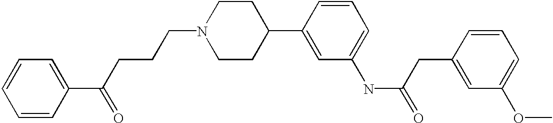 | 21 |
| 77 | 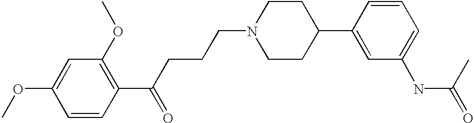 | 630 |
| 78 | 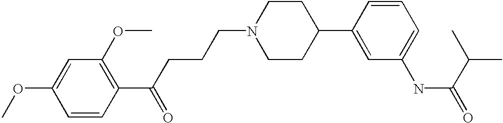 | 52 |
| 79 | 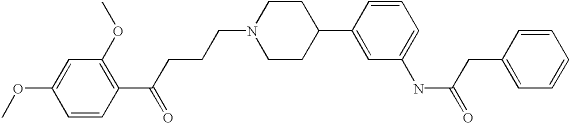 | 1036 |
| 80 | 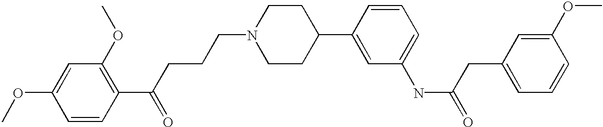 | 67 |
| 81 | 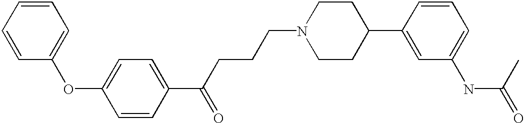 | 463 |
| 82 | 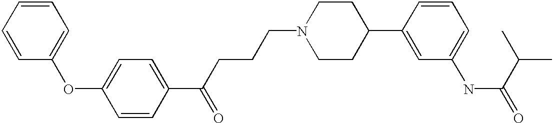 | 192 |
| 83 | 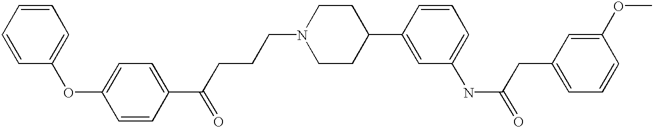 | 91 |
| 84 | 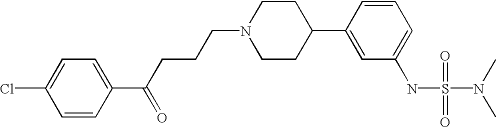 | 511 |
| 85 | 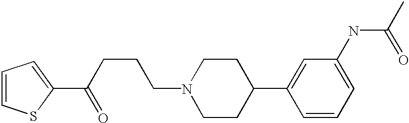 | 654 |
| 86 | 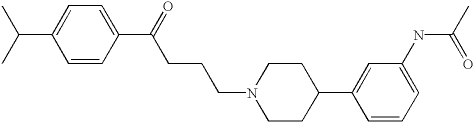 | 382 |
| 87 | 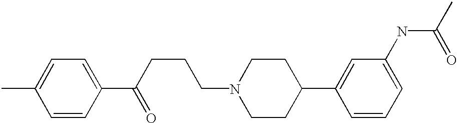 | 362 |
| 88 | 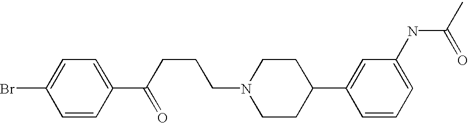 | 160 |
| 89 | 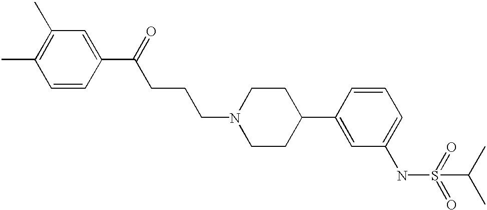 | 615 |
| 90 | 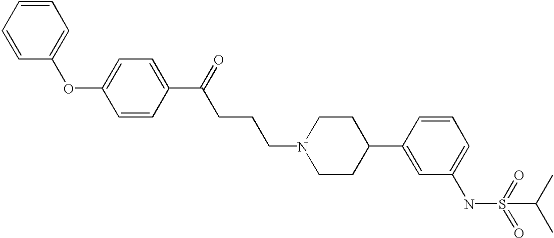 | 651 |
| 91 | 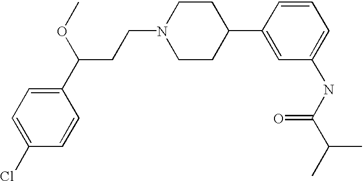 | 11.5 |
| 92 | 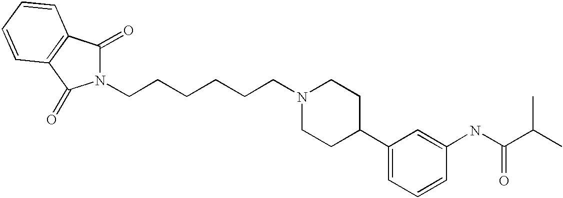 | 62 |
| 93 | 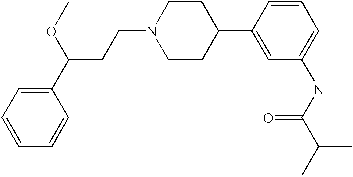 | 29.1 |
| 94 | 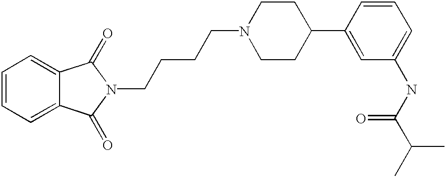 | 18.2 |
| 95 | 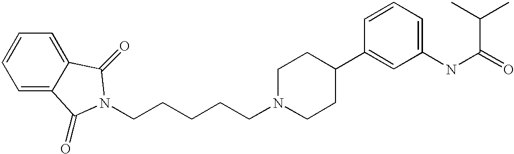 | 11.8 |
| 96 | 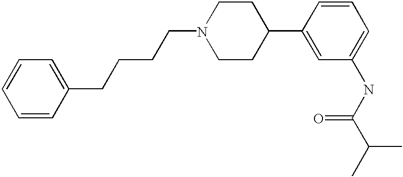 | 50 |
| 97 | 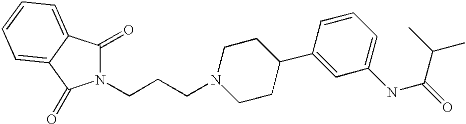 | 946 |
| 98 | 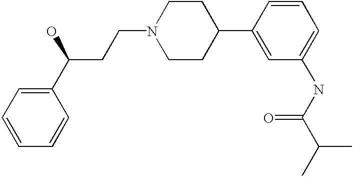 | 118 |
| 99 | 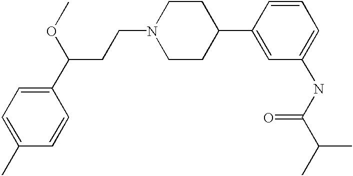 | 12 |
| 100 | 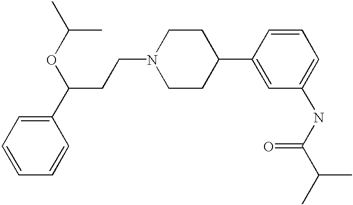 | 11.5 |
| 101 | 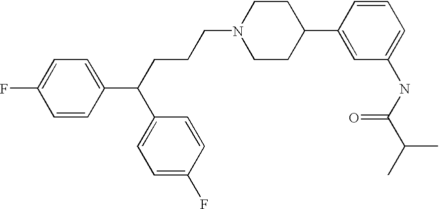 | 1.6 |
| 102 | 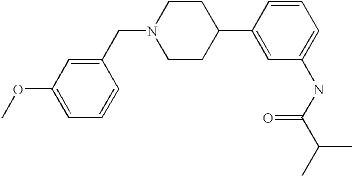 | 187 |
| 103 | 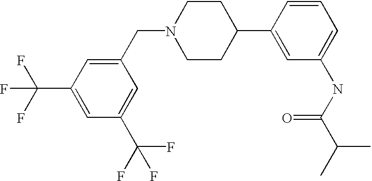 | 52 |
| 104 | 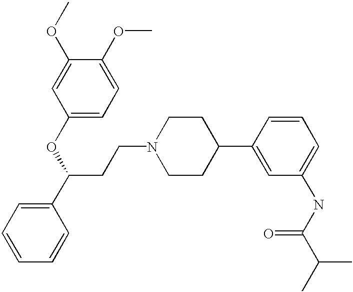 | 6.7 |
| 105 | 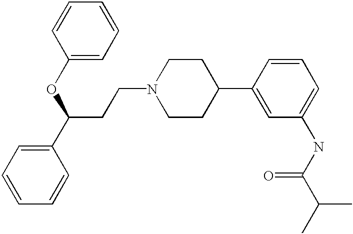 | 7.1 |
| 106 | 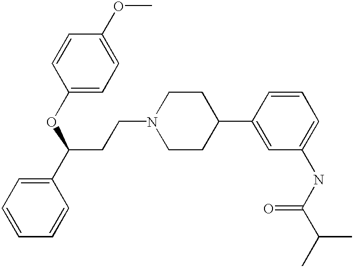 | 3.9 |
| 107 | 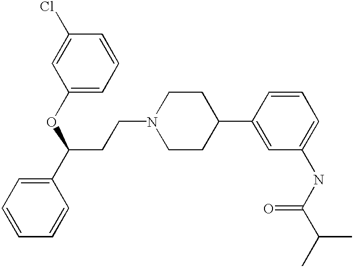 | 3.1 |
| 108 | 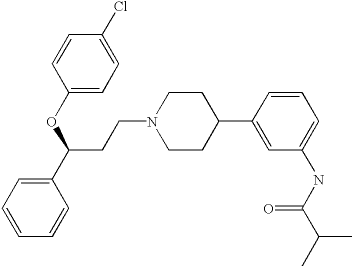 | 3.8 |
| 109 | 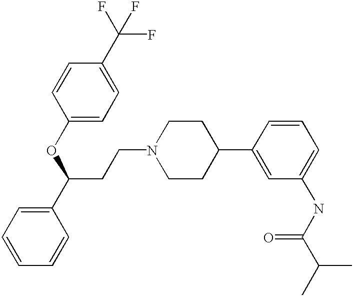 | 7.1 |
| 110 | 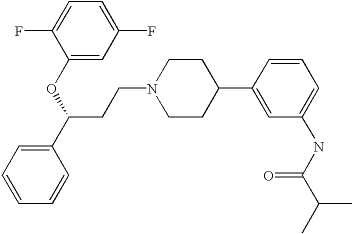 | 4.9 |
| 111 | 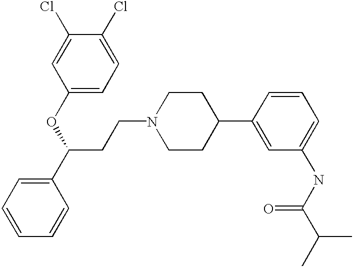 | 5 |
| 112 | 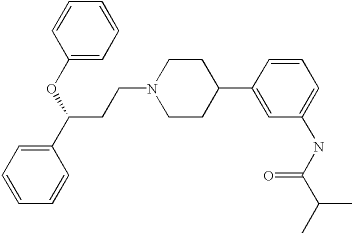 | 22.3 |
| 113 | 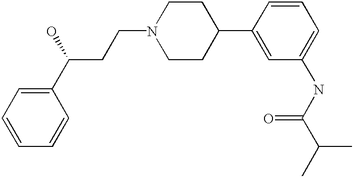 | 16.6 |
| 114 | 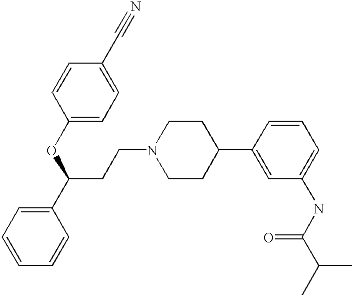 | 2.01 |
| 115 | 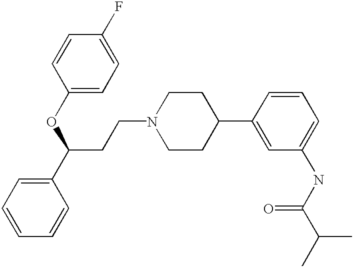 | 12.9 |
| 116 | 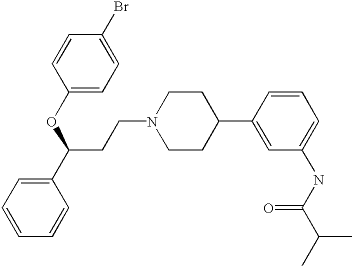 | 0.923 |
| 117 | 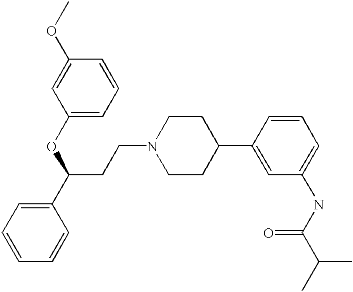 | 13.6 |
| 118 | 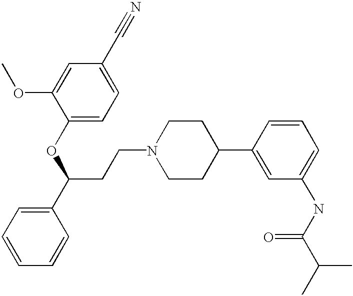 | 12.8 |
| 119 | 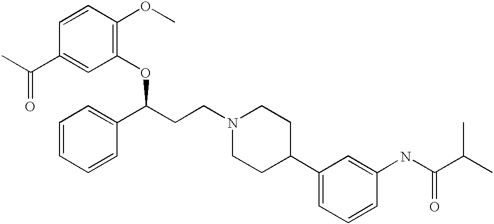 | 22.4 |
| 120 | 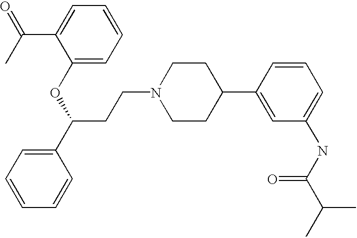 | 14.8 |
| 121 | 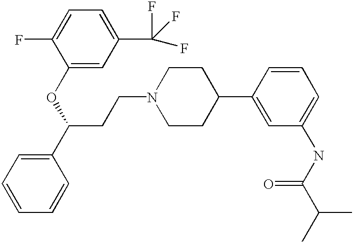 | 17 |
| 122 | 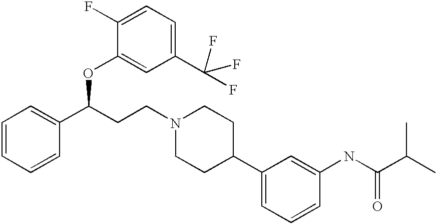 | 3.3 |
| 123 | 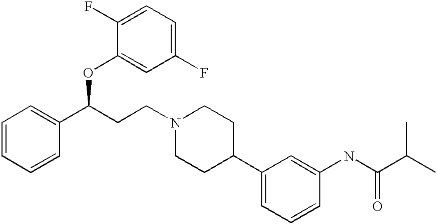 | 5.9 |
| 124 | 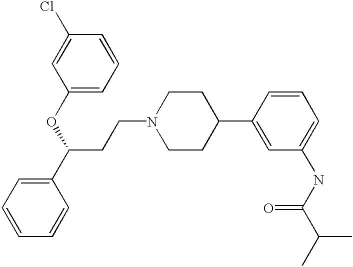 | 9.3 |
| 125 | 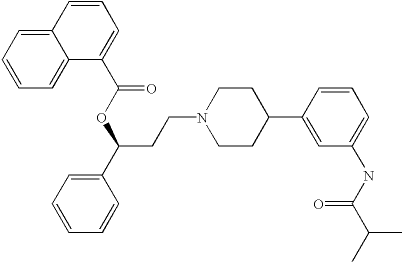 | 32.5 |
| 126 | 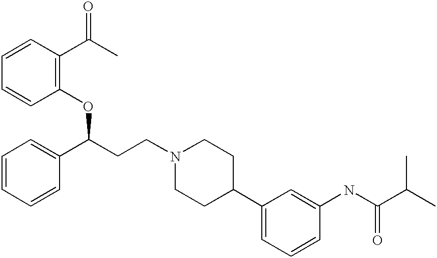 | 50 |
| 127 | 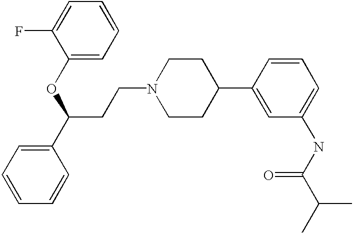 | 6.6 |
| 128 | 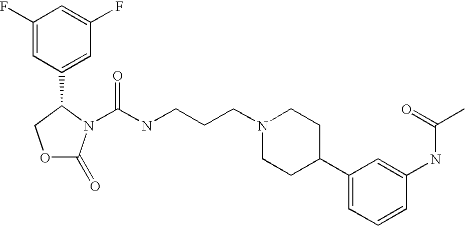 | 31.4 |
| 129 | 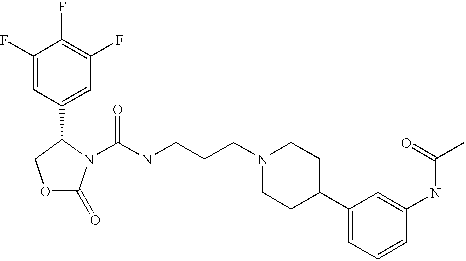 | 22.3 |
| 130 | 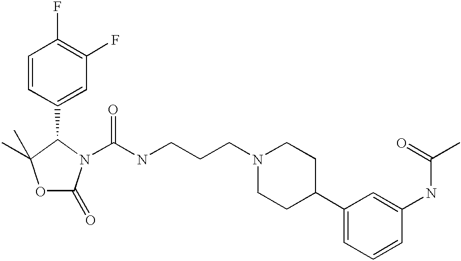 | 48.6 |
| 131 | 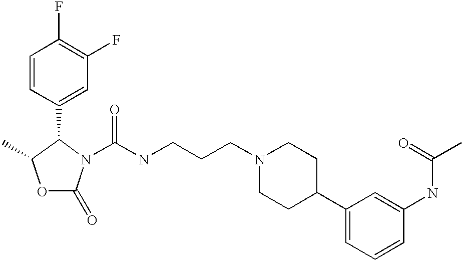 | 11.8 |
| 132 | 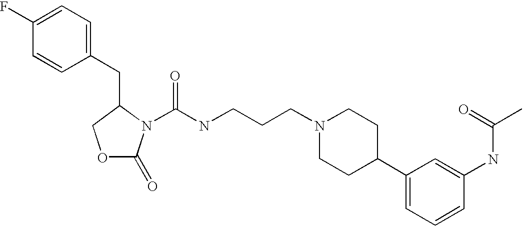 | 44.6 |
| 133 | 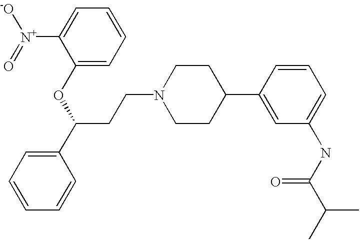 | 25.7 |
| 134 | 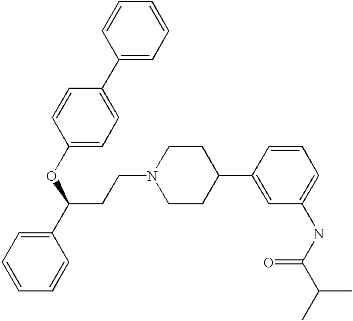 | 22.2 |
| 135 | 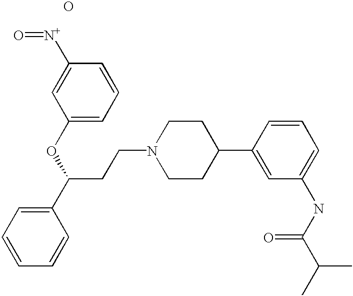 | 19.4 |
| 136 | 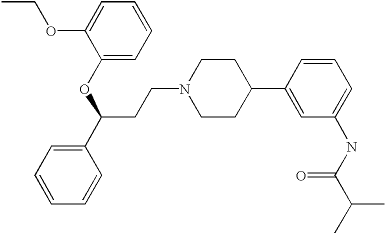 | 14.3 |
| 137 | 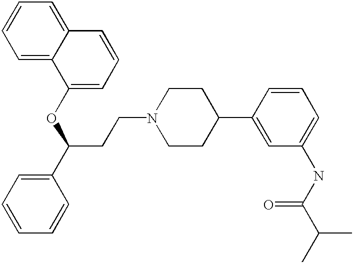 | 377 |
| 138 | 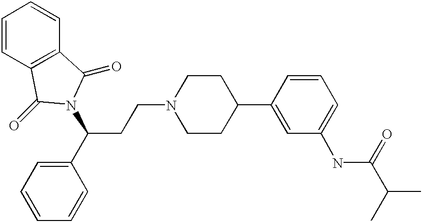 | 11.2 |
| 139 | 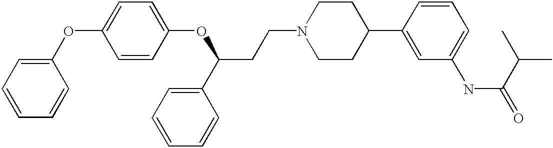 | 48.1 |
| 140 | 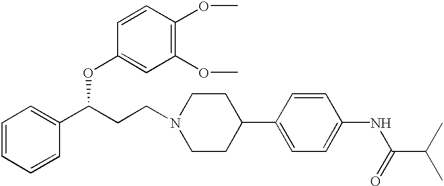 | 121 |
| 141 | 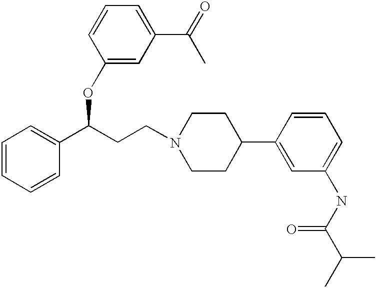 | 3.2 |

In Vivo Models

Materials and Methods

1. Effects on MCH-Stimulated Food Intake

To determine if an MCH1 antagonist could attenuate MCH-stimulated food intake, the effect of an i.p. dose of Compound 10 on food intake induced by intracerebral ventricularly administered MCH was measured.

Animals

Adult male albino Wistar rats (Charles River Laboratories, NY) were housed individually and maintained on a 12 h light dark cycle and given free access to Purina rat chow and water. Rats were pretreated with chlorpromazine (3 mg/kg, i.p.) and anesthesized with Ketamine HCl (120 mg/kg, i.m.). A stainless steel cannula (22 gauge, Plastics One, Roanoke, Va.) was implanted stereotaxically (Kopf Instrumetns, Tujunda, Calif.) aimed at the third ventricle using the following coordinates: incisor bar (+5 mm), 3.0 mm posterior to Bregma, 1.5 mm lateral and angled 10° towards the sagittal suture, and 9 mm from the top of the skull. The cannula was secured to the skull by 4 anchor screws with dental acrylic. Animals were allowed 10 days to recover before testing began.

Testing Paradigm

Rats were habituated to the testing paradigm over several days in which the food bin was removed from the home cage, and preweighed food pellets were placed on the floor of the animal's cage at 3-6 hours into the light cycle. Animals were considered to have met a baseline criterion of minimal food intake (<1 g over 2 hours) after 2 consecutive days. Rats were then administered vehicle (artificial CSF, 5 ul, 1 ul/15 sec) into the third ventricle via a stainless steel internal cannula (28-gauge, Plastics One) connected to a Hamilton microsyringe by polyethylene tubing. Food was introduced on the floor of the cage immediately after injection and intake was assessed 30, 60 and 120 min after. After verifying low levels of intake following vehicle administration, MCH (3 nmol, 5 ul) was microinjected into the third ventricle and food intake assessed as above. Subgroups of these rats were then tested with the following pairs of injections in counterbalanced order with a minimum of 4 days elapsing between injection conditions: a) DMSO (1%, i.p.) 10 min prior to MCH (third ventricle, 3 nmol, 5 ul, n=11), b) Compound 10 (1 mg/kg, i.p.) 10 min before MCH (third ventricle, 3 nmol, 5 ul, n=8), and c) Compound 10 (10 mg/kg, i.p.) 10 min before MCH (third ventricle, 3 nmol, 5 ul, n=6). Food was introduced immediately after the second injection and intake assessed as above.

2. Effects of MCH1 Antagonists on Body Weight

Male Long Evans rats (Charles River) weighing 180-200 grams at the start of experiments were housed in pairs (osmotic minipump experiment) or groups of four (i.p. injections) on a 12 hour light/dark cycle with free access to food and water.

For studies involving osmotic minipumps, rats were anesthesized with isoflurane (Aerrane, Baxter Pharmaceutical) and an osmotic mimpump (model 2ML2, Alzet, Palo Alto, Calif.) filled with either vehicle (20% DMSO), Compound 10 (19.2 mg/ml in 20% DMSO) or d-fenfluramine (Sigma, St. Louis Mo.; 11.5 mg/ml in 20% DMSO) was implanted subcutaneously into the mid scalpular region. At these concentrations, rats received continuous infusions of 10 mg/kg/day of Compound 10 or 6 mg/kg/day of d-fenfluramine.

For studies involving i.p. injections, drugs were administered twice daily, once 1 hour before the dark cycle and once 2 hours after lights on. All rats were weighed daily after the morning injection. Overall results were analyzed by two-way ANOVA, data for each time point were analyzed by one-way ANOVA followed by post hoc Student-Newman-Keuls test.

3. Effects of MCH1 Antagonists on Consumption of Sweetened Condensed Milk

Male Sprague Dawley rats (Charles River) weighing 180-200 grams at the start of experiments were housed in groups of four on a 12 hour light/dark cycle with free access to food and water. For 7 days, rats were weighed, placed in individual cages and allowed to drink sweetened condensed milk (Nestle, diluted 1:3 with water) for 20 min 2-5 hours into the light cycle. The amount of milk consumed was determined by weighing the milk bottle before and after each drinking bout. On the test day, rats received i.p. injections of Compound 10 (3, 10 or 30 mg/kg in 0.01% lactic acid), vehicle (0.01% lactic acid) of d-fenfluramine (3 mg/kg in 0.01% lactic acid) 30 min prior to exposure to milk. The amount of milk consumed on the test day (in mls milk/ kg body weight) was compared to the baseline consumption for each rat determined on the previous 3 days. Data was analyzed using a two-tailed unpaired t-test.

4. Forced Swim Test (FST)

The procedure used in this study was similar to that previously described (Porsolt, et al., 1978), except the water depth (30 cm in this procedure). The greater depth in this test prevented the rats from supporting themselves by touching the bottom of the cylinder with their feet. Swim sessions were conducted by placing rats in individual plexiglass cylinders (46 cm tall×20 cm in diameter) containing 23-25° C. water 30 cm deep (Porsolt, et al. used a depth of only 15 cm; also, see Detke, et al., 1995). Two swim tests were conducted always between 1200 and 1800 hours: an initial 15-min pretest followed 24 h later by a 5-minute test. Drug treatments were administered 30 minutes before the 5-minute test period. All other test sessions were conducted between 1300 to 1700 hours. Following all swim sessions, rats were removed from the cylinders, dried with paper towels and placed in a heated cage for 15 minutes and returned to their home cages. All test sessions were videotaped using a Panasonic color video camera and recorder for scoring later.

Animals

Male Sprague-Dawley rats (Taconic Farms, N.Y.) were used in all experiments. Rats were housed in pairs and maintained on a 12:12-h light-dark cycle. Rats were handled for 5 minutes each day for 5 days prior to behavioral testing.

Behavioral Scoring

The rat's behavior was rated at 5 second intervals during the 5 minute test as one of the following:

1. Immobility—rat remained floating in the water without struggling and was only making those movements necessary to keep its head above water;

2. Climbing—rat was making active movements with its forepaws in and out of the water, usually directed against the walls;

3. Swimming—rat was making active swimming motions, more than necessary to merely maintain its head above water, e.g. moving around in the cylinder; and

4. Diving—entire body of the rat was submerged.

All of the behavior scoring was done by a single rater, who was blind to the treatment condition.

Drug Administration

Animals were randomly assigned to receive a single i.p. administration of Compound 10 (3, 10 or 30 mg/kg, dissolved in 5% lactic acid), fluoxetine (10 mg/kg, dissolved in distilled water) or vehicle (equal mixture of 5% lactic acid and distilled water) 30 minutes before the start of the 5 minute test period. All injections were given using 1 cc tuberculin syringe with 26 3/8 gauge needles (Becton-Dickinson, VWR Scientific, Bridgeport, N.J.). The volume of injection was 1 ml/kg.

The effect of 10 mg/kg of fluoxetine was utilized in the FST as a positive control.

Data Analysis

The forced swim test data (immobility, swimming, climbing, diving) were subjected to a randomized, one-way ANOVA and post hoc tests conducted using the Student-Newman-Keuls test. The data were analyzed using the GBSTAT program, version 6.5 (Dynamics Microsystems, Inc., Silver Spring, Md., 1997). All data are presented as means ±S.E.M.

5. Social Interaction Test (SIT)

Rats were allowed to acclimate to the animal care facility for 5 days and were housed singly for 5 days prior to testing. Animals were handled for 5 minutes per day. The design and procedure for the Social Interaction Test was carried out as previously described by Kennett, et al. (1997). On the test day, weight matched pairs of rats (±5%), unfamiliar to each other, were given identical treatments and returned to their home cages. Animals were randomly divided into 5 treatment groups, with 5 pairs per group, and were given one of the following i.p. treatments: Compound 10 (3, 10 or 30 mg/kg), vehicle (1 ml/kg) or chlordiazepoxide (5 mg/kg). Dosing was 1 hour prior to testing. Rats were subsequently placed in a white perspex test box or arena (54×37×26 cm), whose floor was divided up into 24 equal squares, for 15 minutes. An air conditioner was used to generate background noise and to keep the room at approximately 74° F. All sessions were videotaped using a JVC camcorder (model GR-SZ1, Elmwood Park, NJ) with either TDK (HG ultimate brand) or Sony 30 minute videocassettes. All sessions were conducted between 1:00-4:30 P.M. Active social interaction, defined as grooming, sniffing, biting, boxing, wrestling, following and crawling over or under, was scored using a stopwatch (Sportsline model no. 226, 1/100 sec. discriminability). The number of episodes of rearing (animal completely raises up its body on its hind limbs), grooming (licking, biting, scratching of body), and face washing (i.e. hands are moved repeatedly over face), and number of squares crossed were scored. Passive social interaction (animals are lying beside or on top of each other) was not scored. All behaviors were assessed later by an observer who was blind as to the treatment of each pair. At the end of each test, the box was thoroughly wiped with moistened paper towels.

Animals

Male albino Sprague-Dawley rats (Taconic Farms, N.Y.) were housed in pairs under a 12 hr light dark cycle (lights on at 0700 hrs.) with free access to food and water.

Drug Administration

Compound 10 was dissolved in 5% lactic acid. Chlordiazepoxide (purchased from Sigma Chemical Co., St. Louis, Mo.) was dissolved in distilled water. The vehicle was an equal mixture of 5% lactic acid and distilled water. All drug solutions were made up 10 minutes prior to injection and the solutions were discarded.

Data Analysis

The social interaction data (time interacting, rearing and squares crossed) were subjected to a randomized, one-way ANOVA and post hoc tests conducted using the Student-Newman-Keuls test. The data were subjected to a test of normality (Shapiro-Wilk test). The data were analyzed using the GBSTAT program, version 6.5 (Dynamics Microsystems, Inc., Silver Spring, Md., 1997). All data are presented as means ±S.E.M.

Results and Discussion

Cloning and Sequencing

Discovery of an Expressed Sequence Tag (EST) F07228 in GENEML Homologous to FB41a

A BLAST search of GENEMBL with a Synaptic Pharmaceutical Corporation proprietary sequence, FB41a, resulted in the identification of an EST (accession number F07228) with a high degree of homology to FB41a and somatostatin, opiate and galanin receptors.

Construction and Screening of a Human Hippocampal cDNA Library

A human hippocampal cDNA library containing a total of 2.2×10⁶independent clones with a mean insert size of 3.0 kb was prepared in the expression vector pEXJ.BS. The library was plated on agar plates (ampicillin selection) and glycerol stocks for 450 pools of 5000 independent clones were prepared. Primary glycerol stocks were also grouped together in groups of approximately 10 to create superpools.

Cloning of the Full-Length Sequence of MCH1

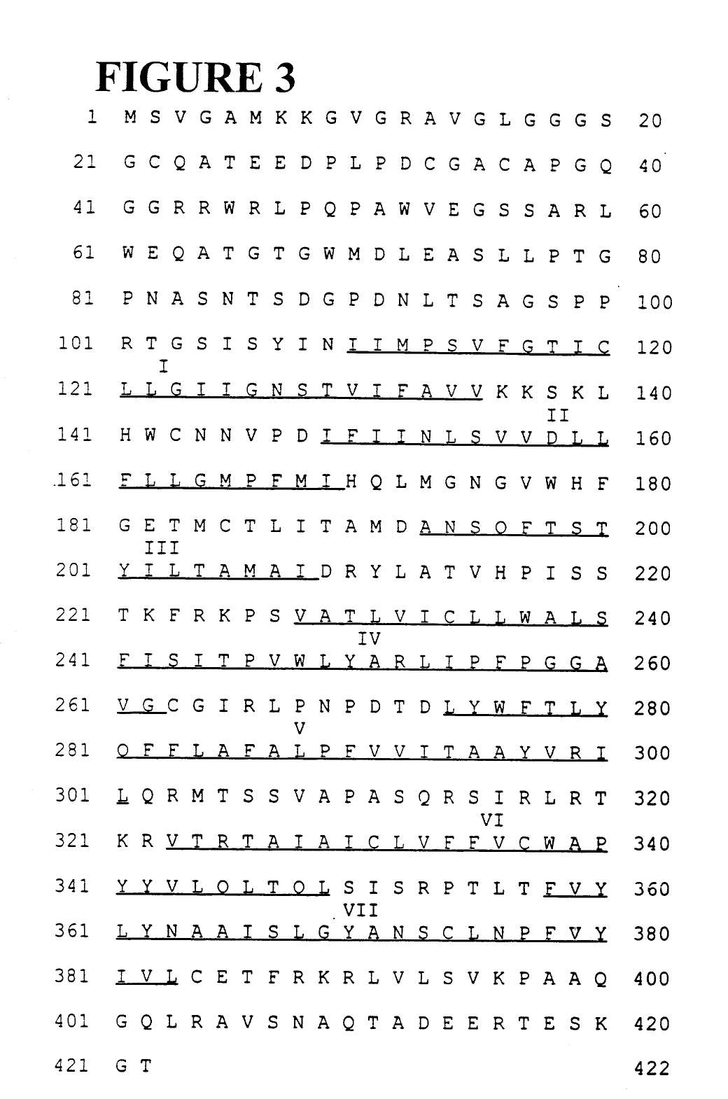

Glycerol stocks of the superpools and primary pools from the human hippocampal cDNA library were screened by PCR with F07228 specific primers T579 and T580. One positive primary pool 490, was successively divided into subpools, amplified in LB medium overnight and screened by PCR using primers T579 and T580. One positive subpool, 490-4-10-23 was plated on agar plates (ampicillin selection), and colonies were transferred to nitrocellulose membranes (Schleicher and Schuell, Keene, N.H.). Filters were hybridized for two days under high stringency conditions with 106 cpm/ml of a ³²P-labeled cDNA probe, T581, designed against the F07228 EST sequence. Filters were washed and apposed to Biomax MS film (Kodak). Seven positive colonies were picked, streaked on LB-AMP plates, and grown overnight. Two individual colonies from each of the original seven were picked and subjected to vector-anchored PCR using the following primer pairs: T95, T580 and T94, T579. One positive colony, G1, was amplified overnight in TB and processed for plasmid purification. This plasmid was designated TL230 and sequenced on both strands. Nucleotide and peptide sequence analysis were performed with GCG programs (Genetics Computer Group, Madison, Wis.). A HindIII-KpnI fragment of TL230 was subcloned into the mammalian expression vector pEXJ, and named TL231. The largest open reading frame in this construct contains 1266 nucleotides (FIG. 1), which is predicted to encode a protein of 422 amino acids (FIG. 2). There are three in-frame methionines in the amino terminus which could result in a protein of 422, 417 or 353 amino acids. Hydropathy analysis of the protein is consistent with a putative topography of seven transmembrane domains, indicative of the G protein-coupled receptor family (FIG. 3). TL231 has been named MCH1.

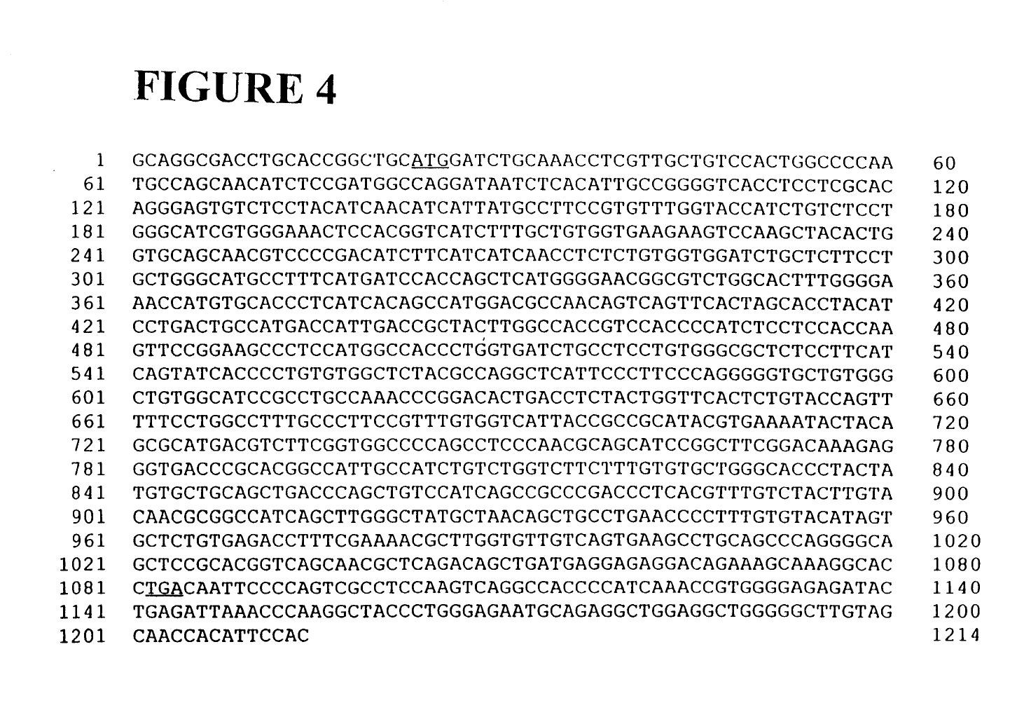

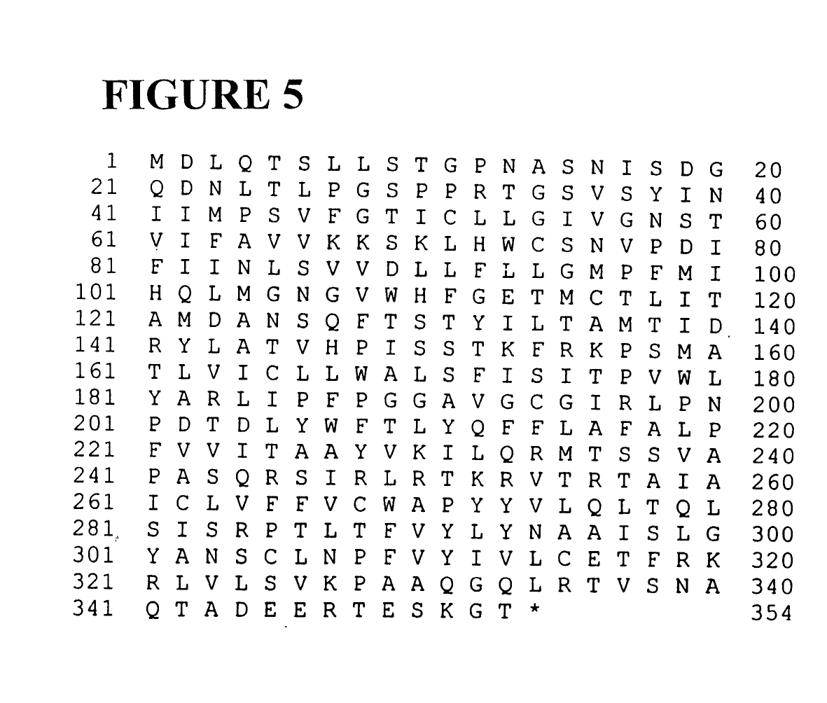

Database analysis of the sequence of MCH1 revealed that it was most similar to somatostatin receptors. Further database analysis revealed a Genbank submission (accession number AF008650, deposited on Oct. 1, 1997) which appears to be the rat homologue of TL231. AF008650 is 69 nucleotides shorter than MCH1 at the 5′end, and predicts a different initiating methionine. FIGS. 4 and 5 illustrate the nucleotide and amino acid sequence for the rat MCH1 receptor, respectively.

Inositol Phosphate Response of MCH1-Transfected Cells

The expression vector (pEXJ) containing the MCH1 cDNA was transfected by electroporation into Cos-7 cells in combination with an expression vector (PEXJ) containing the G_{α16} subunit. After plating and labeling with [³H]-myo-inositol, the transfectants were challenged with a ligand library that included, among other things, melanin concentrating hormone (MCH) (10 μM final concentration) and then assayed for inositol phosphate (IP) formation. In five out of the seven screens, cells transfected with MCH1 (with G_{α16}) gave an approximately 1.4-fold increase in IP production as compared to cells transfected with G_{α16} alone when challenged with MCH.

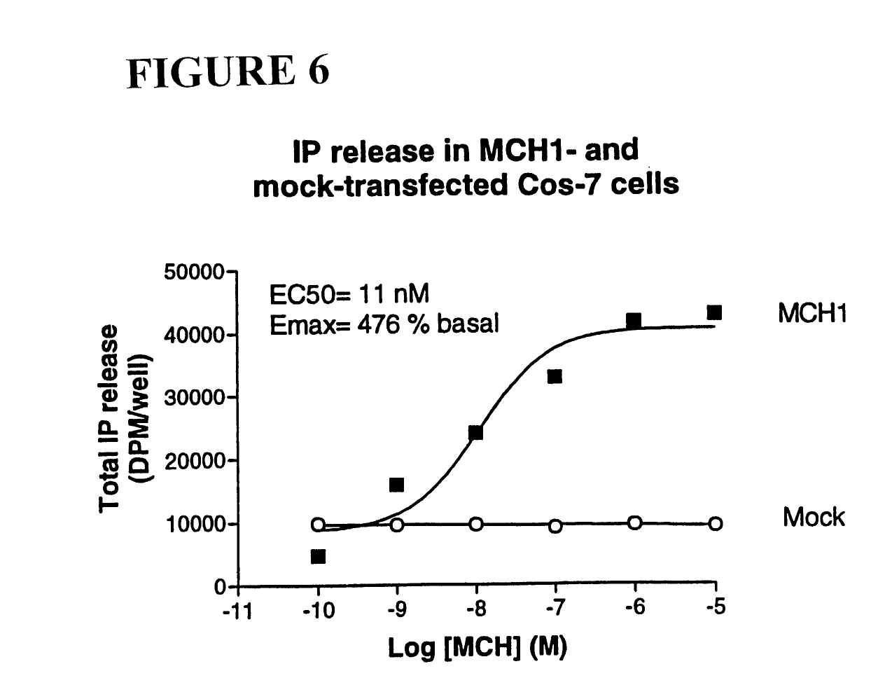

Subsequent experiments demonstrated that 10 μM MCH was able to stimulate IP release 3.4-fold over basal levels in Cos-7 cells transfected with MCH1 alone, suggesting that this receptor couples through the G_{q} signaling pathway. The IP response was shown to be dose-dependent to MCH with an EC₅₀ value of 9.3±1.7 nM (n=2) and an Eₘₐₓ of approximately 400% basal (404±72) (FIG. 6).

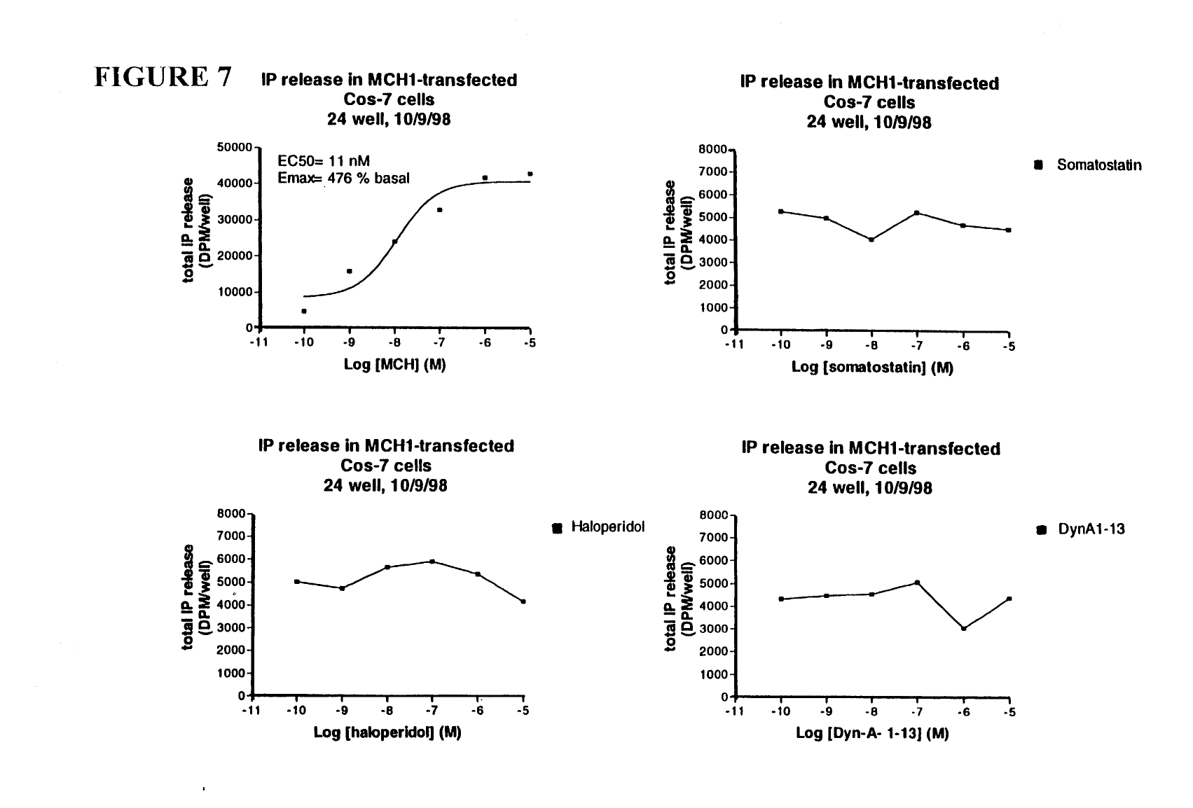

Several additional compounds were tested for their ability to activate MCH1. No dose-responsiveness of inositol phosphate formation could be detected in Cos-7 cells transfected with MCH1 when challenged with somatostatin, haloperidol, or dynorphin A1-13, discounting the possibility that MCH1 encodes a somatostatin-like or opioid-like or sigma-like GPCR subtype (FIG. 7)

Microphysiometric Response of MCH1-Transfected Cells to MCH

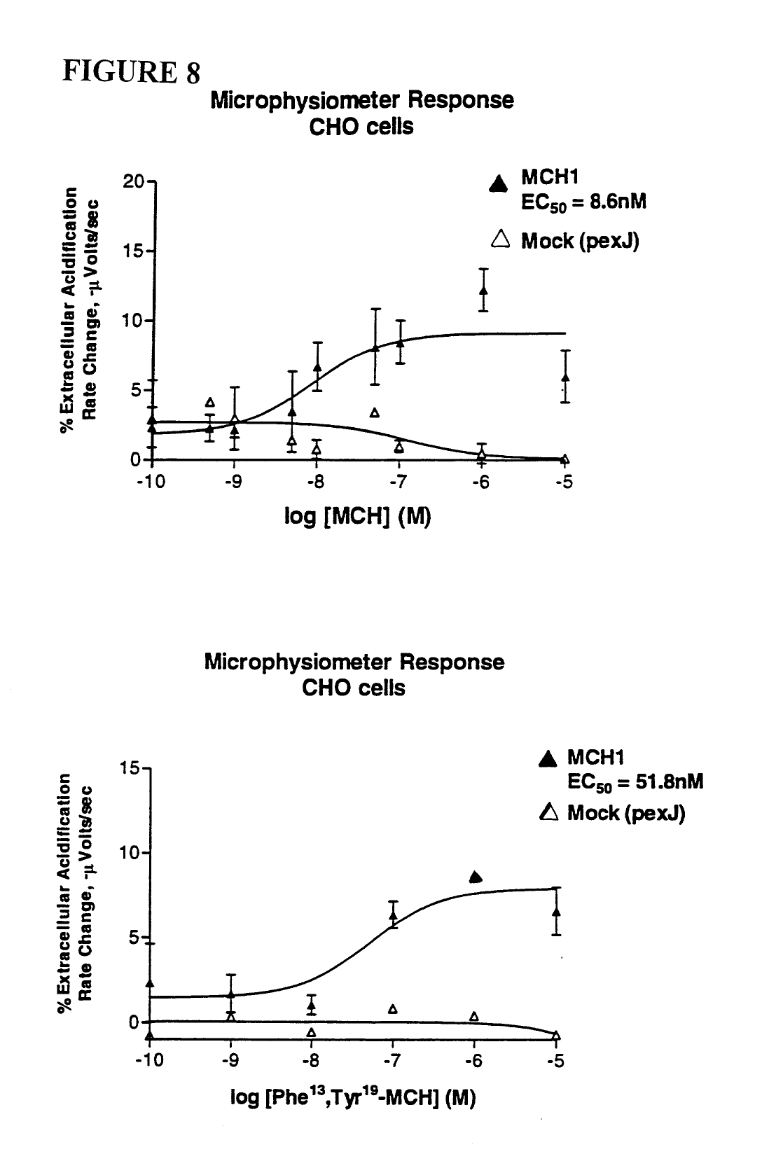

CHO cells were transiently transfected with MCH1 using lipofectant, challenged with increasing concentrations of MCH or Phe¹³, Tyr¹⁹-MCH, and subsequently monitored for changes in extracellular acidification rates. Both ligands produced a dose-dependent increase in acidification rate with an EC_{═}value of 8.6 nM for MCH and 51.8 nM for Phe¹³,Tyr¹⁹-MCH. Neither native CHO cells or mock (pEXJ) transfected CHO cells exhibited a change in acidification rate when exposed to MCH or Phe¹³,Tyr¹⁹-MCH (FIG. 8).

Transcriptional Response of MCH1-Transfected Cells

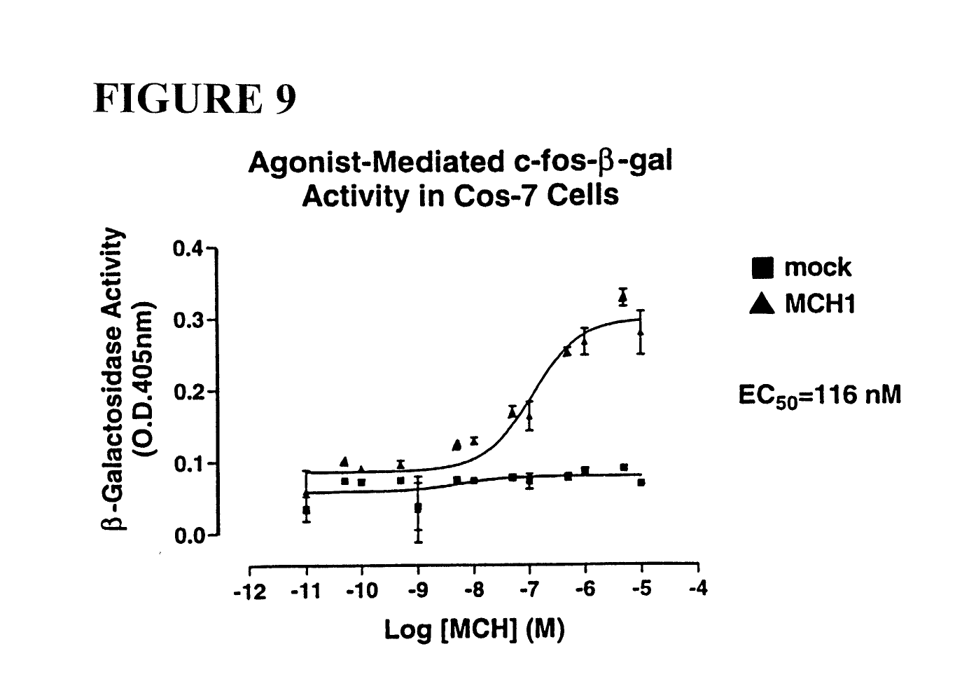

Cos-7 cells were transiently transfected with MCH1 and a c-fos-β-gal reporter construct by the DEAE-dextran method. The cells were challenged with assorted drugs, including MCH, and transcriptional activity measured by calorimetric assay of β-galactosidase protein expression. Initial single dose challenges with MCH at a concentration of 10 μM stimulated c-fos-regulated transcriptional activity approximately 3.9-fold over cells challenged with medium only. Cells transfected with only the c-fos-β-gal construct showed no response to MCH. Subsequent experimentation showed the transcription activation response to be dose-dependent to MCH with an EC₅₀ value of 116 nM (FIG. 9).

Binding of [¹²⁵I]Phe ¹³,Tyr¹⁹-MCH in MCH1-Transfected Cells

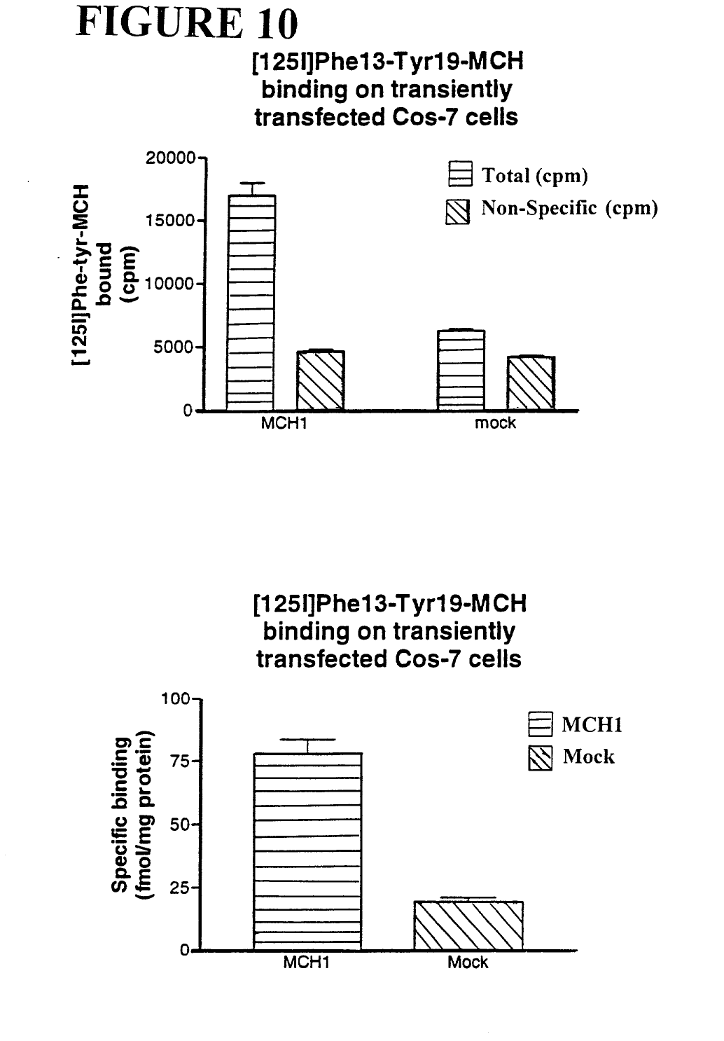

Membranes harvested from Cos-7 cells transfected with MCH1 by the DEAE-dextran method exhibited specific binding for [¹²⁵I]Phe¹³-Tyr¹⁹-MCH (about 80 fmol/mg membrane protein) over mock-transfected cells (about 20 fmol/mg membrane protein) at 0.1 nM radioligand concentration. Specific [¹²⁵I]Phe¹³-Tyr¹⁹-MCH binding was about 70% of total binding at a radioligand concentration of 0.1 nM (FIG. 10).

Localization of mRNA Encoding Human MCH1 Receptors

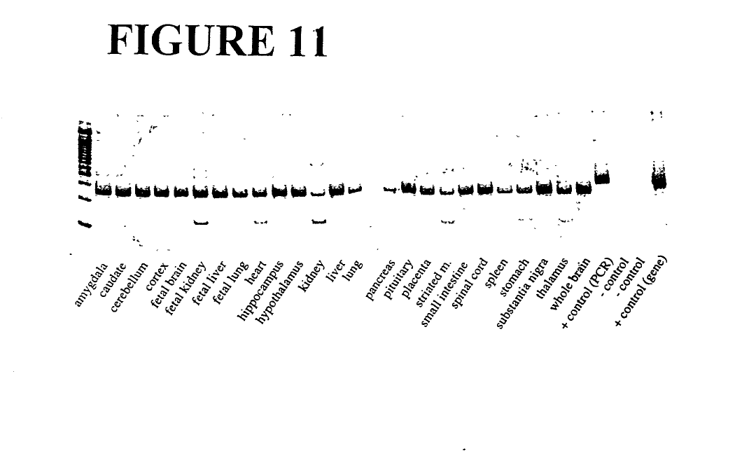

RT-PCR was used to assess the presence of MCH1 receptor encoding message in mRNA samples isolated from a variety of human tissues (Table 1, FIG. 11). After amplification, PCR reactions were size fractionated on 10% polyacrylamide gels, and stained with SYBR Green I. Images were analyzed using a Molecular Dynamics Storm 860 workstation. The amplified band corresponding to MCH1 receptor (490 base pairs) is indicated (arrow). RT-PCR analysis indicates the distribution of mRNA encoding human MCH1 receptor is widespread throughout all tissues assayed, including both central nervous system tissue and peripheral organs. This widespread distribution implies broad regulatory functions that involve nervous system as well as endocrine mechanisms.

TABLE 1
Distribution of mRNA coding for human MCH1
receptors.

| | human | |
|---|---|---|
| Region | MCH 1 | Potential applications |
| liver | +++ | Diabetes |
| kidney | +++ | Hypertension, Electrolyte |
| | | balance |
| lung | +++ | Respiratory disorders, asthma |
| heart | +++ | Cardiovascular indications |
| small intestine | +++ | Gastrointestinal disorders |
| striated muscle | +++ | Musculoskeletal disorders |
| pituitary | +++ | Endocrine/neuroendocrine |
| | | regulation |
| whole brain | +++ | |
| amygdala | +++ | Depression, phobias, anxiety, |
| | | mood disorders |
| cerebral cortex | +++ | Sensory and motor |
| | | integration, cognition |
| hippocampus | +++ | Cognition/memory |
| hypothalamus | +++ | appetite/obesity, |
| | | neuroendocrine regulation |
| spinal cord | +++ | Analgesia, sensory modulation |
| | | and transmission |
| cerebellum | +++ | Motor coordination |
| thalamus | +++ | sensory integration |
| substantia | +++ | Modulation of dopaminergic |
| nigra | | function. Modulation of motor |
| | | coordination. |
| caudate-putamen | +++ | Modulation of dopaminergic |
| | | function |
| fetal brain | +++ | Developmental disorders |
| fetal lung | +++ | Developmental disorders |
| fetal kidney | +++ | Developmental disorders |
| fetal liver | +++ | Developmental disorders |

The cloning of the gene encoding the human MCH1 receptor has provided the means to explore its physiological role by pharmacological characterization, and by Northern and in situ mapping of its mRNA distribution. Further, the availability of the DNA encoding the human MCH1 receptor will facilitate the development of antibodies and antisense technologies useful in defining the functions of the gene products in vivo. Antisense oligonucleotides which target mRNA molecules to selectively block translation of the gene products in vivo have been used successfully to relate the expression of a single gene with its functional sequelae. Thus, the cloning of this receptor gene provides the means to explore its physiological role in the nervous system and elsewhere, and may thereby help to elucidate structure/function relationships within the GPCR superfamily.

The presence of three different potential starting codons in the cDNA sequence of TL231 opens the question of which of the possible transcripts yields an active MCH receptor. In order to establish whether a transcript of the first and second starting codons of TL231 encode a functional human MCH receptor, methionines 6 and 70 of TL231 were mutated to alanine (construct R114; See FIG. 12). The third methionine at position 70 was also mutated to an alanine (construct R106; See FIG. 12). Transfections of TL231, R106 or R114 into COS-7 cells all resulted in MCH-mediated increases of intracellular calcium, as measured by a fluorescent intensity plate reader in cells loaded with the calcium dye fluo-3 (FLIPR, Molecular Devices). As shown in Table 2, COS-7 cells transfected with TL231, R106, R114 and B0120 showed dose-related mobilization of intracellular calcium when exposed to increasing concentrations of MCH with similar maximal responses and EC50 values. These data demonstrate that transcripts starting at the first and/or second and third methionine of TL231 encode a functional human MCH receptor.

TABLE 2

| | |
|---|---|
| | Response to Melanin Concentrating |
| Transfected | Hormone* |

| Construct | EC50 (nM) | Max. Response (RFU**) |
|---|---|---|
| TL231 | 60, 12 | 3,535, 14,000 |
| R114 | 98, 9 | 2,267, 1,550 |
| R106 | 85, 55 | 4642, 2000 |
| BO120 | 12, 3.5 | 30,000, 25,000 |

*Results from two independent experiments
**RFU = relative fluorescence units

Discovery of MCH1 Receptor Antagonists

The intracellular calcium response to MCH in COS-7 cells transfected with MCH1 was used as an assay to identify MCH1 receptor antagonists. Compounds of known chemical structure were added at a concentration of 1 mM to COS-7 cells expressing MCH1 loaded with the calcium indicator fluo-3, and the fluorescence intensity was measured in the absence and presence of 500 nM MCH. MCH1 antagonist compounds were identified by their ability to inhibit the MCH-elicited response. The identified compounds were then tested at 12 different concentrations (between 1e-4 to 3e-10 M) to determine the dose that inhibited the response of 500 nM MCH by 50% (IC50). From the IC50 values, the antagonist potency (Kb)was derived using the Cheng-Prussof correction (Lazareno and Birdsall, 1993). Table 3 exemplifies compounds that were found to have a Kb lower than 500 nM.

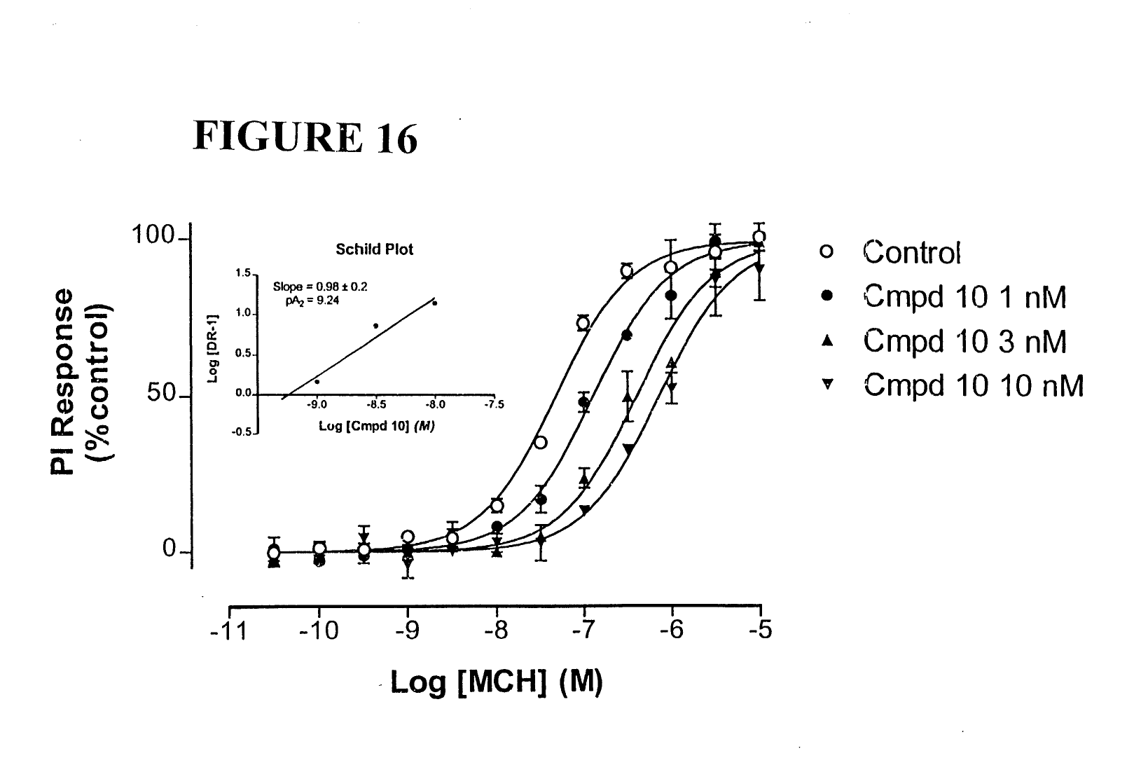

Among the compounds tested, Compound 10 was identified as the most potent antagonist of the human MCH1 receptor. The antagonism of Compound 10 was further characterized with inositol phosphate response in Cos-7 cells transfected with the human MCH1 receptor. As shown in FIG. 16, in the presence of 1, 3, and 10 nM of Compound 10 parallel displacement of the dose-response curves for MCH were observed, suggesting the presence of a competitive antagonist. The Schild analysis of the dose-response yielded a pA2=9.24 with a slope close to unity. This value correlates closely with the Kb=0.3 nM determined using the intracellular calcium mobilization assay.

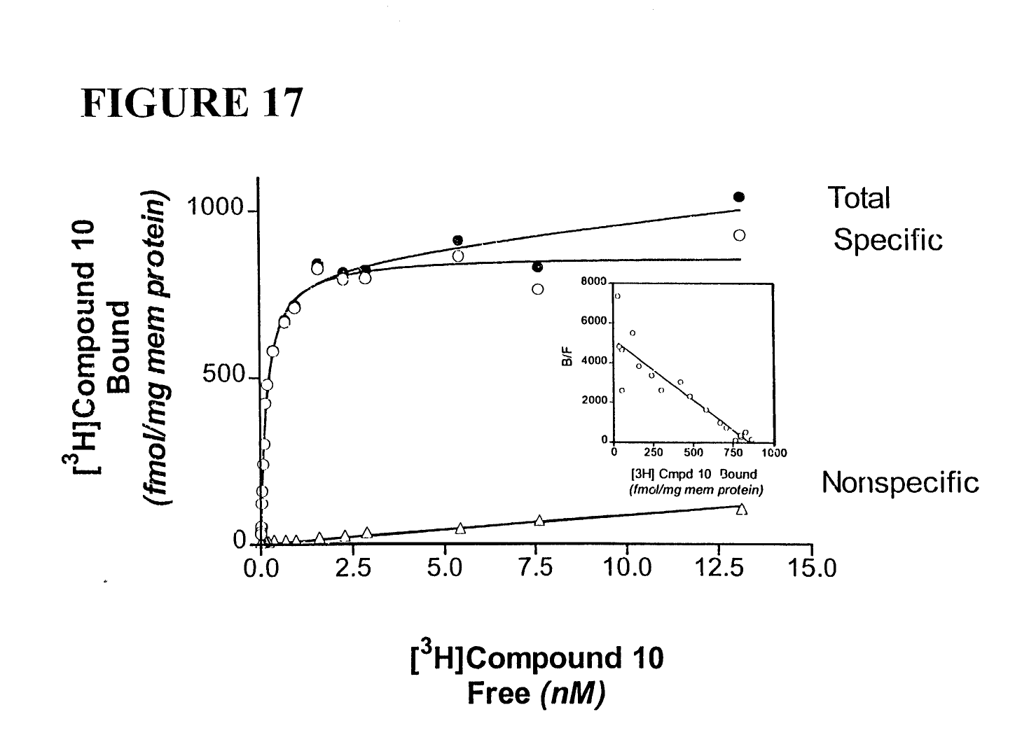

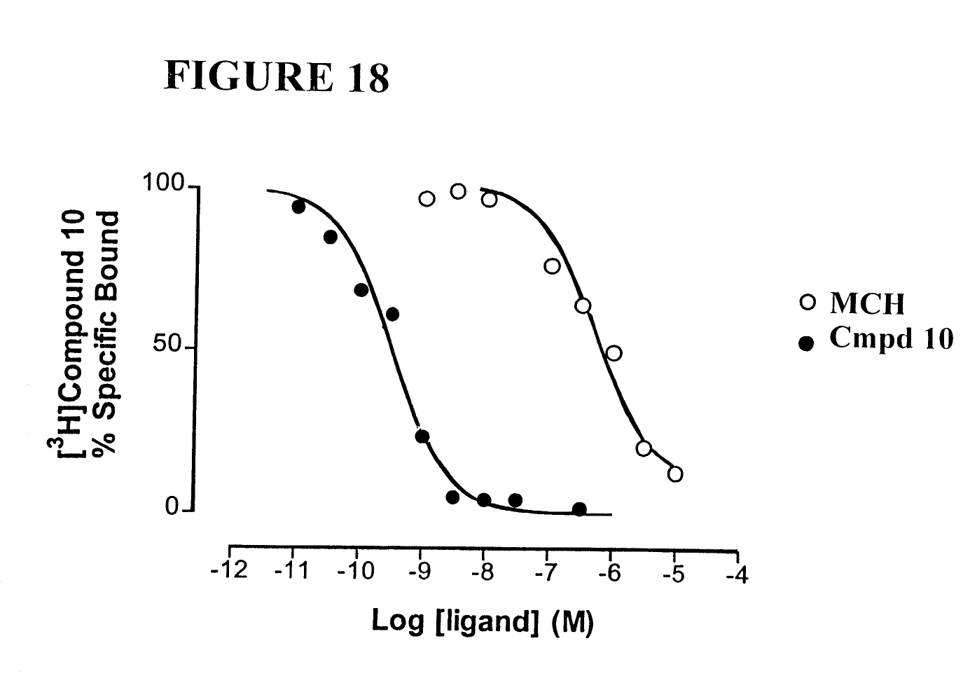

Given the high affinity of Compound 10 for the MCH1 receptor, a tritiated analog of this compound was synthesized. [3H]Compound 10 was tested for its ability to bind to membrane preparations of cells expressing the human MCH1 receptor. As shown in FIG. 17, addition of increasing concentrations of [3H]Compound 10 in the absence (Total) and presence of 10 mM Compound 10 (Nonspecific) resulted in saturable specific binding to membrane preparations of Cos-7 cells transfected with MCH1. The Scatchard analysis of the binding data estimated a Kd=0.18 nM for [3H]Compound 10 and maximum number of binding sites (Bmax)=870 fmol/mg protein (see inset of FIG. 17). In competition binding assays using membrane preparations of Cos-7 cells transfected with MCH1, Compound 10 and MCH completely displaced the specific binding of [3H]Compound 10 with IC50's of 0.33 and 511 nM respectively (FIG. 18). In non-transfected Cos-7 cells the binding of [3H]Compound 10 was not displaced by MCH or unlabeled Compound 10 up to 10 mM. These data together demonstrate that [3H]Compound 10 is a specific and high affinity radioligand for the MCH1 receptor.

As described in the Background of the Invention, compounds that block the effects of MCH on its receptor can potentially be used for the treatment of eating disorders and obesity. The study of the regulation of body weight and food intake point towards an important role for hypothalamic circuits and their neurotransmitters together with circulating metabolic signals, such as leptin, in energy homeostasis (Elmquist et al., 1999). Hypothalamic neurons that mediate appetite-driving (orexigenic) effects include those that use neuropeptide Y, MCH, galanin, and orexin as transmitters. Conversely, neural elements that mediate orexigenic signals include among others those that use serotonin (5HT), alpha-MSH, and CART (cocaine and amphetamine regulated transcript) as transmitters. Recent advances in the molecular cloning of the receptors for some of these transmitter molecules, together with the characterization of their pharmacological properties, has enabled the identification of numerous potential targets for therapeutic intervention. In the case of NPY, the evidence suggests that both NYP1 and NPY5 receptors are involved in mediating the orexigenic effects of NPY (Inui, 1999). The antiorexigenic effects of alpha-MSH are mediated by the MC4 receptor (Fan et al, 1997). In addition, an important role for the antiorexigenic effects of serotoninergic agonists was suggested by the obese phenotype of mice with targeted deletion of the 5HT2C receptor gene (Nonogaki, 1998). However, the interaction between orexigenic and anorexigenic pathways in the hypothalamus displays considerable redundancy typical of other biological control mechanisms (Kalra et al., 1999). This complexity suggests that the design of development of drugs that target multiple orexigenic receptors might result in an effective therapeutic modality that could restore the imbalance of energy homeostasis that results in obesity. One such approach could involve the administration of a combination of antagonists of the MCH1 receptor such as those described above together with NPY1, NPY5, or galanin receptor antagonists. An alternative approach is to design a single molecule that antagonizes the orexigenic effects of one or more of MCH, NPY, and galanin by binding to one or more of MCH1, NPY1, NPY5, or galanin receptors. However, in either case the compound(s) should be free of antagonist activity at the MC-4 or 5HT2C receptor, since antagonizing these receptors could result in increased food intake and obesity (Fan et al., 1997). The design of such compounds can be optimized by determining their binding affinity at the recombinant MCH₁, NPY1, NPY2, Gal1, Gal2, Gal3, 5HT2C, and MC-4 receptors. The methods to obtain the cDNA of the receptor, express said receptors in heterologous systems, and carry out assays to determine binding affinity are described in the following publications: human NPY1 (Larhammar et al., 1992), human NPY5 (U.S. Pat. No. 5,602,024, the disclosure of which is hereby incorporated by reference in its entirety into this application), human Gall (Habert-Ortoli et al., 1994), human Gal2 (Smith et al., 1997), human Gal3 (Smith et al., 1998), rat 5HT2C (Julius et al., 1988), and human MC-4 (Gantz et al., 1993). Additionally, the compounds would optimally not bind at the following receptors due to possible side effects: human H1 histamine, human H2 histamine, human alpha-1A adrenergic, human alpha-lD adrenergic, human alpha-2A, human alpha-2B adrenergic, human alpha-2C adrenergic, human dopamine D1, D2, D3, D5 receptors and the β-adrenoceptor. Binding studies for the β-adrenoceptor may be performed according to the method of Riva and Creese, 1989. Binding assays for the remainder of the receptors may be carried out according to the procedures described in U.S. Pat. No. 5,780,485, the disclosure of which is hereby incorporated by reference in its entirety into this application.

As further described in the Background of the Invention, compounds that block the effects of MCH on the MCH1 receptor can potentially be used for the treatment of depression and anxiety. Biogenic amine transmitter molecules that mediate neuronal signals are currently known in the art and include among others serotonin (5HT), norepinephrine (NE), and dopamine (DA). Recent advances in the molecular studies of the mechanisms for these transmitter molecules, together with the characterization of their pharmacological properties, has enabled the identification of numerous potential targets for therapeutic intervention. Inhibitors of the 5HT, NE and DA transporter systems, and inhibitors of the enzyme, monoamine oxidase, have been widely studied and are known to enhance the action of biogenic amine neurotransmitters. The resultant clinically effective antidepressant drugs are known today as TCAs, SSRIs and MAOIs. (Tatsumi et al., 1997; Iversen, 2000).

In the case of MCH, the evidence presented in this invention suggests that GPCR-targeted molecules that bind to and antagonize the MCH1 receptor may be used for the treatment of depression and/or anxiety disorders. However, the MCH1 antagonist(s) should be free of activity at 5HT, NE and DA transporters. Furthermore, the MCH1antagonist(s) should not inhibit the enzymatic activity of monoamine oxidase A (MAO_{A}) or monoamine oxidase B (MAO_{B}) present in the brain (i.e. central MAO). The design of such compounds can be optimized by determining their binding affinity at the 5HT, NE and DA transporters in tissue assays. The design of such compounds can be further optimized by determining their interaction with central MAO_{A} and central MAO_{B}.

Additionally, the MCH1 antagonist(s) would optimally not bind at the following receptors due to possible side effects: human H₁ histamine; human H₂ histamine; human α_{1A} adrenergic, human α_{1B} adrenergic, human α_{1D} adrenergic, human α_{2A} adrenergic, human α_{2B} adrenergic, and human α_{2C} adrenergic; human dopamine D₁, D₂, D₃, D₄, and D₅; and the human 5HT_{1A}, human 5HT_{2A}, hum an 5HT_{1D}, human 5HT_{1E}, human 5HT_{1F}, human ⁵HT_{2A}, rat 5HT_{2C}, human 5HT₄, human 5HT₆, and human 5HT₇ receptors.

Radioligand Binding a ssays and Enzymatic Assays

The methods to obtain the cDNA of the receptors, express said receptors in heterologous systems, and carry out ass ays to determine binding affinity are described as follows.

H uman 5HT_{1B}, 5HT_{1D}, 5HT_{1E}, 5HT_{1F}, and 5HT₇ Receptors:

The cell lysates of LM(tk) clonal cell line stably transfected with the genes encoding each of these 5HT receptorsubtypes were prepared as described above. Cell membranes were suspended in 5OmM TrisHCl buffer (pH 7.4 at 37° C.) containing 10 mM MgCl2, 0.2 mM EDTA, 10 M pargyline, and 0.1% ascorbate. The affinities of compounds were determined in equilibrium competition binding assays by incubation for 30 minutes at 37° C. in the presence of 5 nM [³H]serotonin. Nonspecific binding was determined in the presence of 10 μM serotonin. The bound radioligand was separated by filtration through GF/B filters using a cell harvester.

Human 5HT_{2A} Receptor:

The coding sequence of the human 5HT_{2A} receptor was obtained from a human brain cortex cDNA library, and cloned into the cloning site of pCEXV3 eukaryotic expression vector. This construct was transfected into COS7 cells by the DEAE dextran method (Cullen, 1987). Cells were harvested after 72 hours and lysed by sonication in 5 mM TrisHCl, 5 mM EDTA, pH 7.5. The cell lysates were subjected to centrifugation at 1000 rpm for 5 minutes at 4° C., and the supernatant was subjected to centrifugation at 30,000× g for 20 minutes at 4° C. The pellet was suspended in 50 mM TrisHCl buffer (pH 7.7 at room temperature) containing 10 mM MgSO4, 0.5 mM EDTA, and 0.1% ascorbate. The affinity of compounds at 5HT_{2A} receptors were determined in equilibrium competition binding assays using [³H]ketanserin (1 nM). Nonspecific binding was defined by the addition of 10 μM mianserin. The bound radioligand was separated by filtration through GF/B filters using a cell harvester.

5HT_{1A} Receptor:

The cDNA corresponding to the 5HTlA receptor open reading frames and variable noncoding 5′ and 3′ regions, was cloned into the eukaryotic expression vector pCEXV3. These constructs were transfected transiently into COS7 cells by the DEAEdextran method (Cullen, 1987), and harvested after 72 hours. Radioligand binding assays were performed as described above for the ⁵HT₂A receptor, except that ³H8OHDPAT was used as the radioligand and nonspecific binding was determined by the addition of 10 μM mianserin.

Other 5HT Receptors:

Other serotonin receptor binding assays were performed according to published methods: rat 5HT_{2C} receptor (Julius et al., 1988); and 5HT₆ (Monsma, et al., 1993). The binding assays using the 5HT₄ receptor were performed according to the procedures described in U.S. Pat. No. 5,766,879, the disclosure of which is hereby incorporated by reference in its entirety into this application.

Other receptors: Cell membranes expressing human dopamine D₁, D₂, D₄ and rat D₃ receptors were purchased through BioSignal, Inc. (Montreal, Canada). Binding assays using the histamine H₁ receptor; dopamine receptors; and α_{1A}, α_{1B}, and α₂adrenergic receptors may be carried out according to the procedures described in U.S. Pat. No. 5,780,485, the disclosure of which is hereby incorporated by reference in its entirety into this application. Binding assays using the dopamine D₅ receptor may be carried out according to the procedures described in U.S. Pat. No. 5,882,855, the disclosure of which is hereby incorporated by reference in its entirety into this application. Binding assays for the human α_{1D} adrenergic receptor may be carried out according to the procedures described in U.S. Pat. No. 6,156,518, the disclosure of which is hereby incorporated by reference in its entirety into this application.

The methods to determine binding affinity at native transporters are described in the following publications: 5HT transporter and NE transporter (Owens et al., 1997), and DA transporter (Javitch et al, 1984).

The methods to determine activity at monoamine oxidase enzymes (for example, central MAO_{A} and MAO_{B}) are described by Otsuka and Kobayashi, 1964.

TABLE 3a
Antagonist potency (Kb) at the human MCH1 receptor, and binding affiity (Ki) at
NPY, galanin and 5HT2C receptors.

| | hMCH1 | hNPY1 | hNPY5 | hGALR1 | hGALR2 | hGALR3 | r5HT2C |
|---|---|---|---|---|---|---|---|
| Compound | Kb (nM) | Ki (nM) | Ki (nM) | Ki (nM) | Ki (nM) | Ki (nM) | Ki (nM) |
| 10 | 0.3 | >50000 | >50000 | >50000 | >50000 | >50000 | 29,585 |
| 18 | 1 | >50000 | >50000 | >50000 | >50000 | >50000 | 32,617 |
| 14 | 2 | ND | ND | >50000 | 42,603 | >50000 | 663 |
| 20 | 5 | 27,076 | >50000 | >50000 | >50000 | >50000 | 15,058 |
| 19 | 7 | >50000 | >50000 | >50000 | >50000 | >50000 | 11,720 |
| 29 | 9 | >50000 | 46,075 | >50000 | >50000 | >50000 | >50000 |
| 2 | 18 | ND | ND | >50000 | >50000 | >50000 | 39,837 |
| 6 | 42 | 6,667 | 4,735 | 11,057 | 14,921 | 21,095 | 25,549 |
| 1 | 42 | >50000 | >50000 | >50000 | >50000 | >50000 | >50000 |
| 28 | 60 | >50000 | >50000 | >50000 | >50000 | >50000 | 34,087 |
| 25 | 126 | >50000 | >50000 | >50000 | >50000 | >50000 | 41,009 |
| 37 | 162 | >50000 | >50000 | >50000 | >50000 | >50000 | >50000 |
| 4 | 187 | >50000 | >50000 | >50000 | >50000 | >50000 | 34,798 |
| 26 | 260 | >50000 | >50000 | >50000 | >50000 | >50000 | 2,900 |
| 27 | 279 | >50000 | >50000 | >50000 | >50000 | >50000 | >50000 |
| 13 | 284 | 9,601 | >50000 | 11,262 | 4,727 | 5,985 | 25,030 |
| 30 | 479 | >50000 | >50000 | >50000 | >50000 | >50000 | 8,859 |

TABLE 3b
Antagonist potency (Kb) at the human MCH1 receptor, and binding affiity (Ki) at human
MCH1, NPY1, NPY5, GALR1, GALR2, GALR3, and rat 5HT2C receptors.

| | hMCH1 | hMCH1 * | hNPY1 | hNPY5 | hGALR1 | hGALR2 | hGALR3 | r5HT2C |
|---|---|---|---|---|---|---|---|---|
| Compound | Kb (nM) | Ki (nM) | Ki (nM) | Ki (nM) | Ki (nM) | Ki (nM) | Ki (nM) | Ki (nM) |
| 10 | 0.3 | 0.08 | >50000 | >50000 | >50000 | >50000 | >50000 | 29,585 |
| 19 | 7 | 3 | >50000 | >50000 | >50000 | >50000 | >50000 | 11,720 |
| 18 | 1 | 4 | >50000 | >50000 | >50000 | >50000 | >50000 | 32,617 |
| 20 | 5 | 6 | 27,076 | >50000 | >50000 | >50000 | >50000 | 15,058 |
| 1 | 42 | 40 | >50000 | >50000 | >50000 | >50000 | >50000 | >50000 |
| 2 | 18 | 49 | ND | ND | >50000 | >50000 | >50000 | 39,837 |
| 14 | 2 | 50 | ND | ND | >50000 | 42,603 | >50000 | 663 |
| 4 | 187 | 131 | >50000 | >50000 | >50000 | >50000 | >50000 | 34,798 |
| 13 | 284 | 171 | 9,601 | >50000 | 11,262 | 4,727 | 5,985 | 25,030 |
| 29 | 9 | 350 | >50000 | 46,075 | >50000 | >50000 | >50000 | >50000 |
| 6 | 42 | 463 | 6,667 | 4,735 | 11,057 | 14,921 | 21,095 | 25,549 |

* Binding affinity (Ki) was determined in competition binding assays using membrane preparations of A293 cells expressing the human MCH1 receptor and [3H] Compound 10 as the radioligand.

TABLE 3C
Binding affinities (Ki) at the rat MCH1, human Dopamine D2,
human Histamine H1 and human Alpha-1a Adrenergic receptors.

| | rMCH1 | hD2 | hH1 | hAlpha-1a |
|---|---|---|---|---|
| Compound | Ki (nM) | Ki (nM) | Ki (nM) | Ki (nM) |
| 38 | 1.34 | 2370.49 | 378.91 | 23.82 |
| 39 | 3.33 | 25,142.39 | 5664.28 | 25.48 |
| 40 | 2.72 | 2651.67 | 7123.16 | 8.72 |
| 41 | 0.04 | 9605.79 | 4541.69 | 14.31 |
| 42 | 0.6 | 3274.88 | 7795.17 | 10.91 |
| 43 | 0.23 | 3570.6 | 10,774.03 | 21.86 |
| 44 | 0.09 | 3607.95 | 2594.67 | 11.39 |
| 45 | 14.69 | >50000 | 4432.68 | 6027.11 |
| 46 | 8.16 | >50000 | 2867.82 | 2424.16 |
| 47 | 34.28 | 13,540.64 | 3251.17 | 553.45 |
| 48 | 22.15 | >50000 | 7769.8 | 16,563.03 |
| 49 | 225.47 | ND | ND | ND |
| 50 | 13.74 | 19,796.44 | 7468.06 | 10,385.44 |
| 51 | 0.79 | 6638.62 | 1230.84 | 10.05 |
| 52 | 0.81 | 165.91 | 1428.58 | 200.24 |
| 53 | 50.76 | 3447.38 | 10,387.86 | 2307.97 |
| 54 | 29.87 | 22,966.69 | 11,408.54 | 11,120.65 |
| 55 | 203.74 | ND | ND | ND |
| 56 | 0.26 | 10,399.66 | 7228.37 | 305.44 |
| 57 | 90 | 6092 | 823 | 49 |
| 58 | 3.9 | 2839 | 700 | 32.1 |
| 59 | 768 | ND | ND | ND |
| 60 | 357 | ND | ND | ND |
| 61 | 14.2 | 1139 | 1618 | 9.1 |
| 62 | 274 | ND | ND | ND |
| 63 | 1000 | ND | ND | ND |
| 64 | 627 | ND | ND | ND |
| 65 | 69 | 1430 | 1733 | 26.4 |
| 66 | 2.8 | 862 | 461 | 19.4 |
| 67 | 197 | ND | ND | ND |
| 68 | 84 | 771 | 571 | 57 |
| 69 | 11.9 | 551 | ND | 61 |
| 70 | 167 | ND | ND | ND |
| 71 | 720 | ND | ND | ND |
| 72 | 272 | ND | ND | ND |
| 73 | 342 | ND | ND | ND |
| 74 | 29.5 | 782 | ND | 115 |
| 75 | 506 | ND | ND | ND |
| 76 | 21 | 470 | ND | 41.3 |
| 77 | 630 | ND | ND | ND |
| 78 | 52 | 5181 | 2277 | 284 |
| 79 | 1036 | ND | ND | ND |
| 80 | 67 | 1252 | ND | 127 |
| 81 | 463 | ND | ND | ND |
| 82 | 192 | 1977 | ND | 516 |
| 83 | 91 | 503 | ND | 130 |
| 84 | 511 | ND | ND | ND |
| 85 | 654 | ND | ND | ND |
| 86 | 382 | ND | ND | ND |
| 87 | 362 | ND | ND | ND |
| 88 | 160 | ND | ND | ND |
| 89 | 615 | ND | ND | ND |
| 90 | 651 | ND | ND | ND |
| 91 | 11.5 | 9654 | 2000 | 533 |
| 92 | 62 | 12,026 | 2454 | 1489 |
| 93 | 29.1 | 34,993 | 16,734 | 1087 |
| 94 | 18.2 | >50000 | 6595 | 1592 |
| 95 | 11.8 | >50000 | 6401 | 2937 |
| 96 | 50 | 7451 | 273 | 12.3 |
| 97 | 946 | ND | ND | ND |
| 98 | 118 | ND | ND | ND |
| 99 | 12 | 10,428 | 2560 | 434 |
| 100 | 11.5 | 8673 | 11,092 | 704 |
| 101 | 1.6 | 42.2 | 3.4 | 18 |
| 102 | 187 | ND | ND | ND |
| 103 | 52 | >50000 | 36,907 | >50000 |
| 104 | 6.7 | 735 | 6390 | 452 |
| 105 | 7.1 | 471 | 39.1 | 140 |
| 106 | 3.9 | 1077 | 304 | 161 |
| 107 | 3.1 | 152 | 130 | 33.5 |
| 108 | 3.8 | 244 | 264 | 13.2 |
| 109 | 7.1 | 191 | 1320 | 221 |
| 110 | 4.9 | 83 | 283 | 187 |
| 111 | 5 | 162 | 1100 | 125 |
| 112 | 22.3 | 435 | 32.5 | 55 |
| 113 | 16.6 | 41,994 | 48,658 | 3206 |
| 114 | 20.1 | 390 | 590 | 233 |
| 115 | 12.9 | 262 | 46.9 | 49.1 |
| 116 | 0.923 | 52 | 546 | 22.3 |
| 117 | 13.6 | 281 | 969 | 310 |
| 118 | 12.8 | 319 | 25,320 | 719 |
| 119 | 22.4 | 766 | 25,307 | 1058 |
| 120 | 14.8 | 313 | 6994 | 1142 |
| 121 | 17 | 331 | 9390 | 1720 |
| 122 | 3.3 | 132 | 3473 | 944 |
| 123 | 5.9 | 133 | 2146 | 511 |
| 124 | 9.3 | 66 | 329 | 204 |
| 125 | 32.5 | 46.6 | >50000 | 232 |
| 126 | 50 | 1050 | 7998 | 1521 |
| 127 | 6.6 | 119 | 1710 | 226 |
| 128 | 31.4 | 41,454 | 33,096 | 645 |
| 129 | 22.3 | 41,454 | 6522 | 381 |
| 130 | 48.6 | 39,511 | 1862 | 333 |
| 131 | 11.8 | 19,041 | 2844 | 2469 |
| 132 | 44.6 | 41,454 | 39,710 | 10,965 |
| 133 | 25.7 | 447 | 4178 | 167 |
| 134 | 22.2 | 37.6 | >50000 | 1313 |
| 135 | 19.4 | 244 | 507 | 722 |
| 136 | 14.3 | 833 | 9789 | 620 |
| 137 | 377 | ND | ND | ND |
| 138 | 11.2 | ND | ND | ND |
| 139 | 48.1 | ND | ND | ND |
| 140 | 121 | ND | ND | ND |
| 141 | 3.2 | 2449 | 3816 | 3021 |

TABLE 3d
Antagonist binding affinity (Ki) at the human MCH1 receptor vs. alpha-adrenergic
and dopamine receptors.

| | hMCH1 | hα_{1A} | hα_{1B} | hα_{1D} | hα_{2A} | hα_{2B} | hα_{2C} | hD₁ | hD₂ | rD ₃ | hD ₄ | hD₅ |
|---|---|---|---|---|---|---|---|---|---|---|---|---|
| | Ki | Ki | Ki | Ki | Ki | Ki | Ki | Ki | Ki | Ki | Ki | Ki |
| Compound | (nM) | (nM) | (nM) | (nM) | (nM) | (nM) | (nM) | (nM) | (nM) | (nM) | (nM) | (nM) |
| 10 | 0.08 | 45.8 | 11343 | 12334 | ND | ND | ND | 1928 | * | 5890 | * | 773 |
| 19 | 3 | 2.5 | 104 | 517 | 285 | 349 | 174 | ND | ND | ND | ND | ND |
| 18 | 4 | 1.7 | 418 | 612 | 57 | 74 | 96 | 596 | 624 | 1216 | ND | 2541 |
| 20 | 6 | 0.3 | 407 | 207 | 99 | 97 | 138 | ND | ND | ND | ND | ND |
| 1 | 40 | 2214 | 9033 | 1307 | ND | ND | ND | ND | ND | ND | ND | ND |

TABLE 3e
Antagonist binding affinity (Ki) at the human MCH1 receptor vs.
serotonin and histamine receptors.

| | hMCH1 | h5HT_{1A} | h5HT_{2A} | hH₁ | hH₂ |
|---|---|---|---|---|---|
| | Ki | Ki | Ki | Ki | Ki |
| Compound | (nM) | (nM) | (nM) | (nM) | (nM) |
| 10 | 0.08 | ND | ND | 4875 | * |
| 19 | 3 | 7943 | ND | 11 | ND |
| 18 | 4 | 1245 | 1567 | 31 | 944 |
| 20 | 6 | 2188 | 2818 | 39 | 1905 |

* = >50000
ND = Not determined

Autoradiographic Distribution of MCH1 Receptor Binding Sites In The RAT CNS

Telencephalon

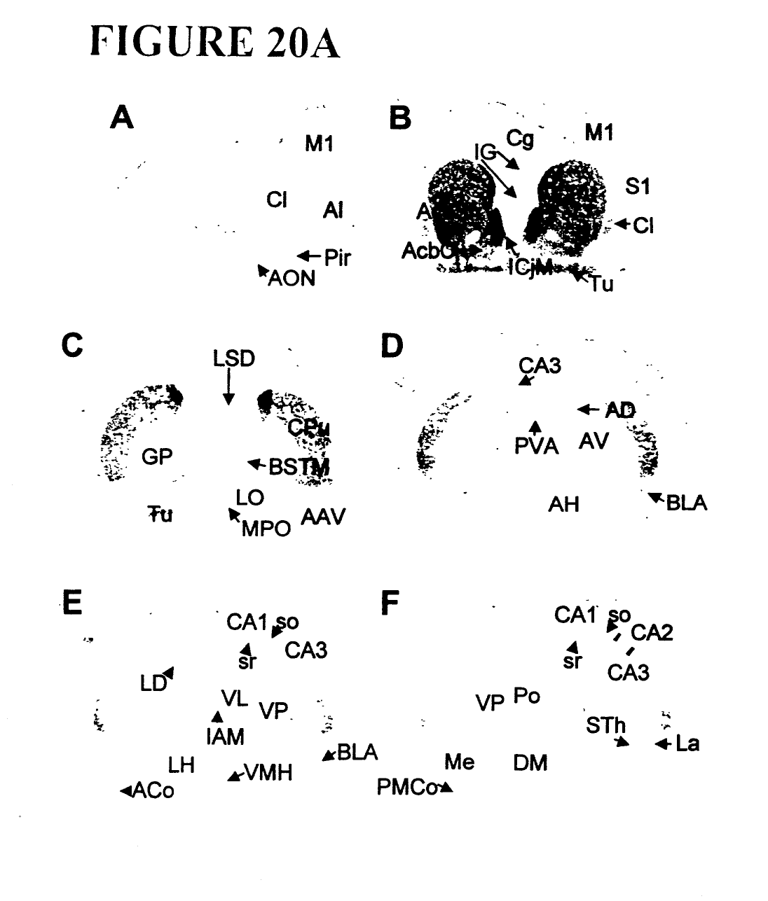

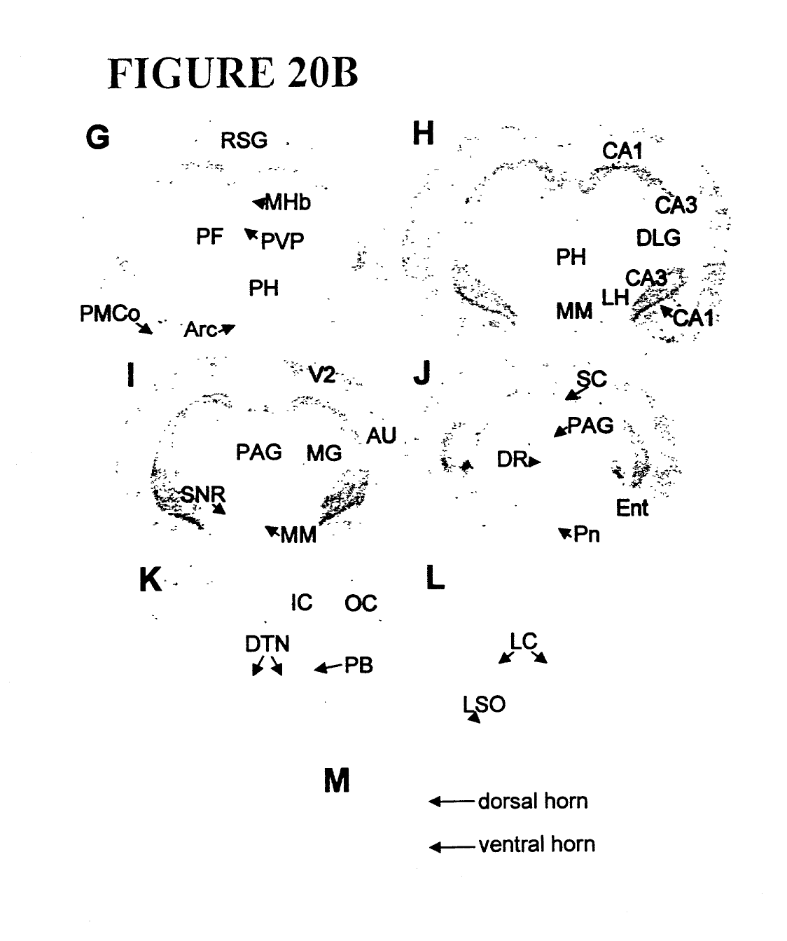

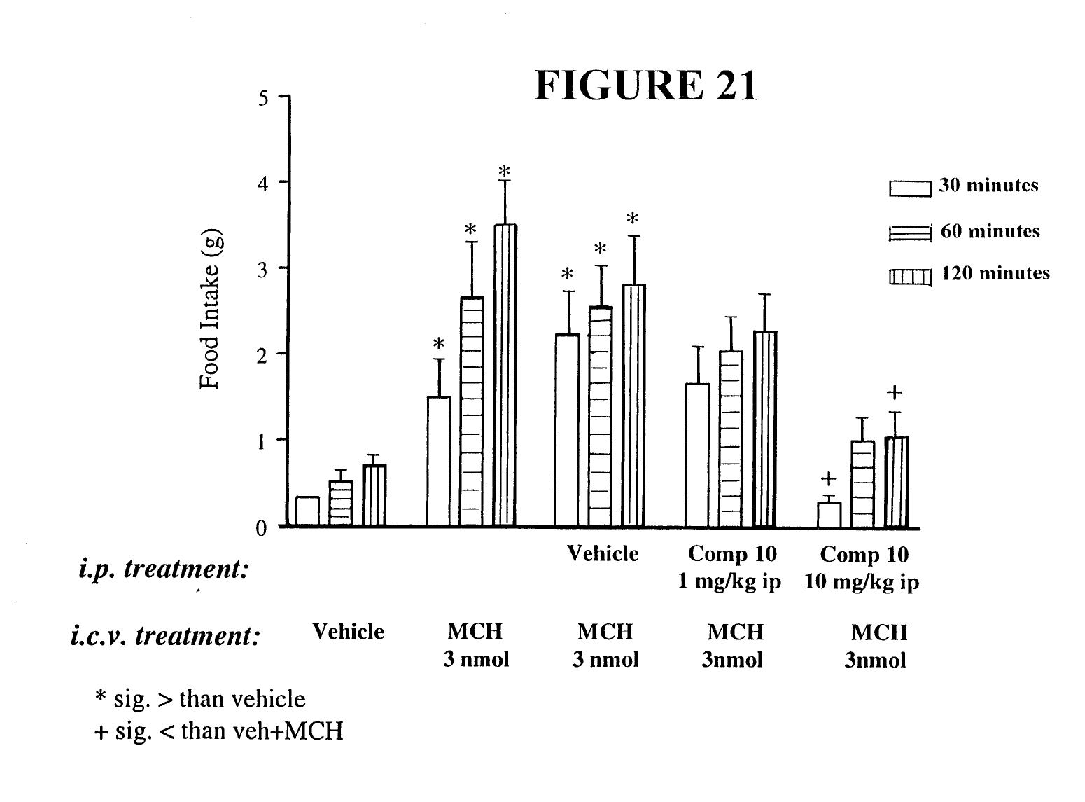

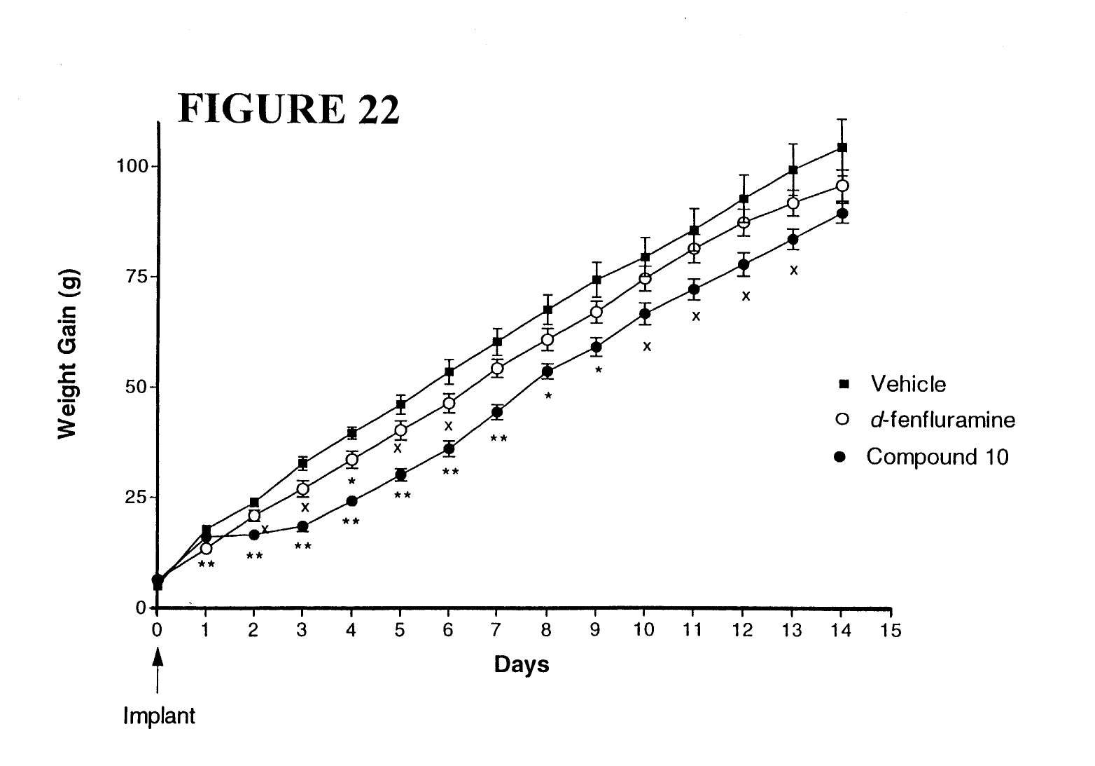

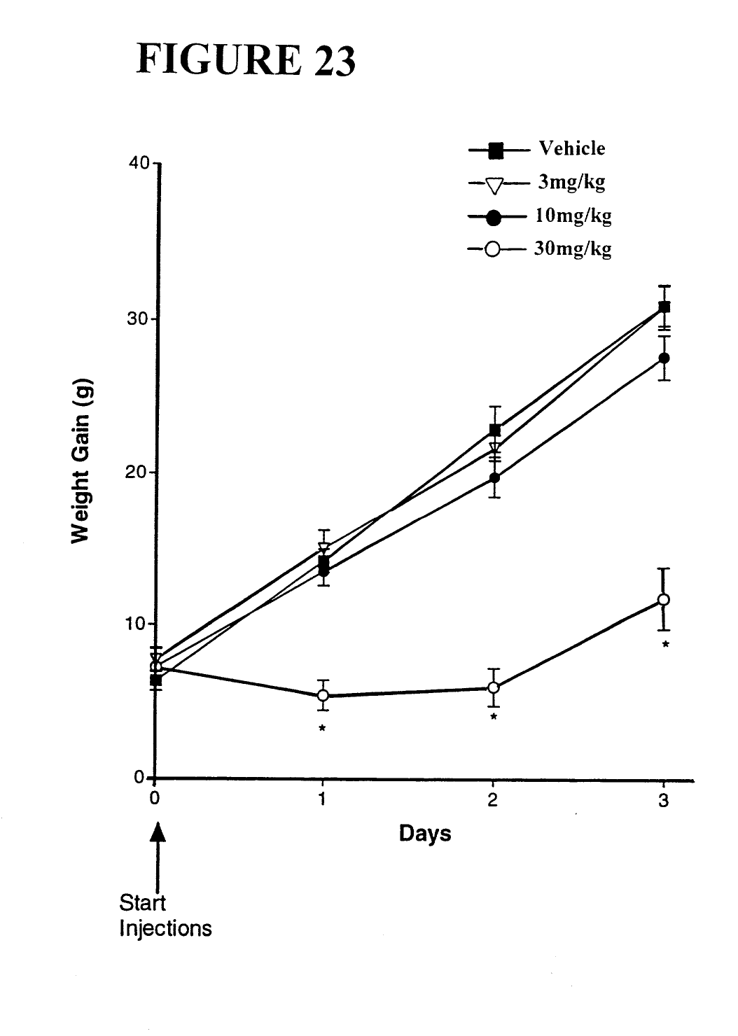

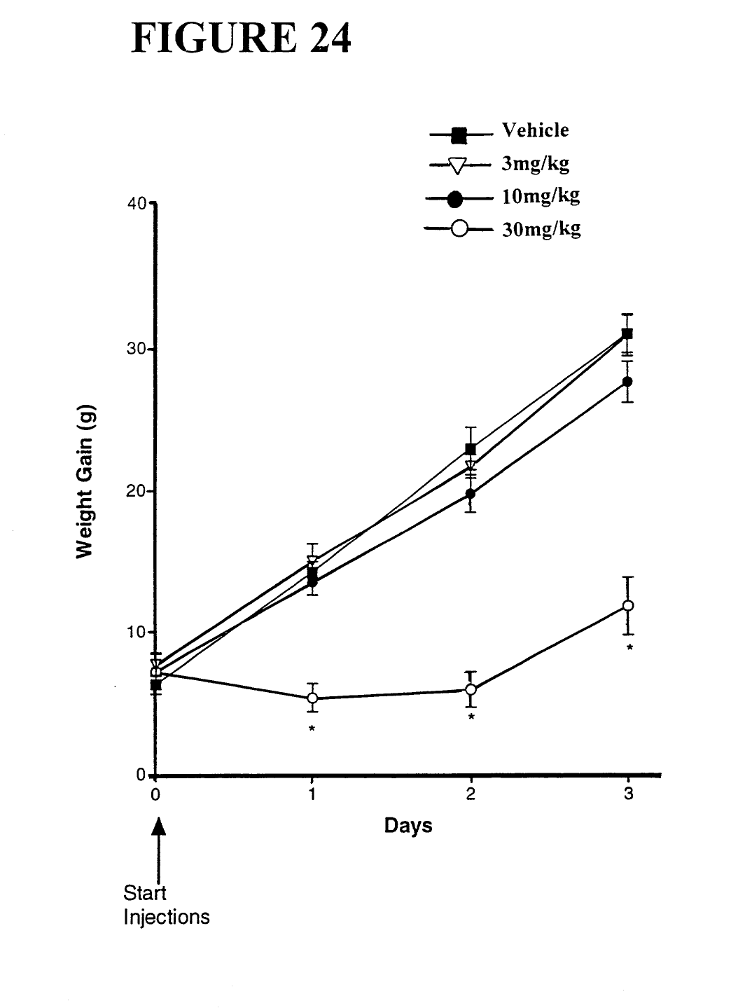

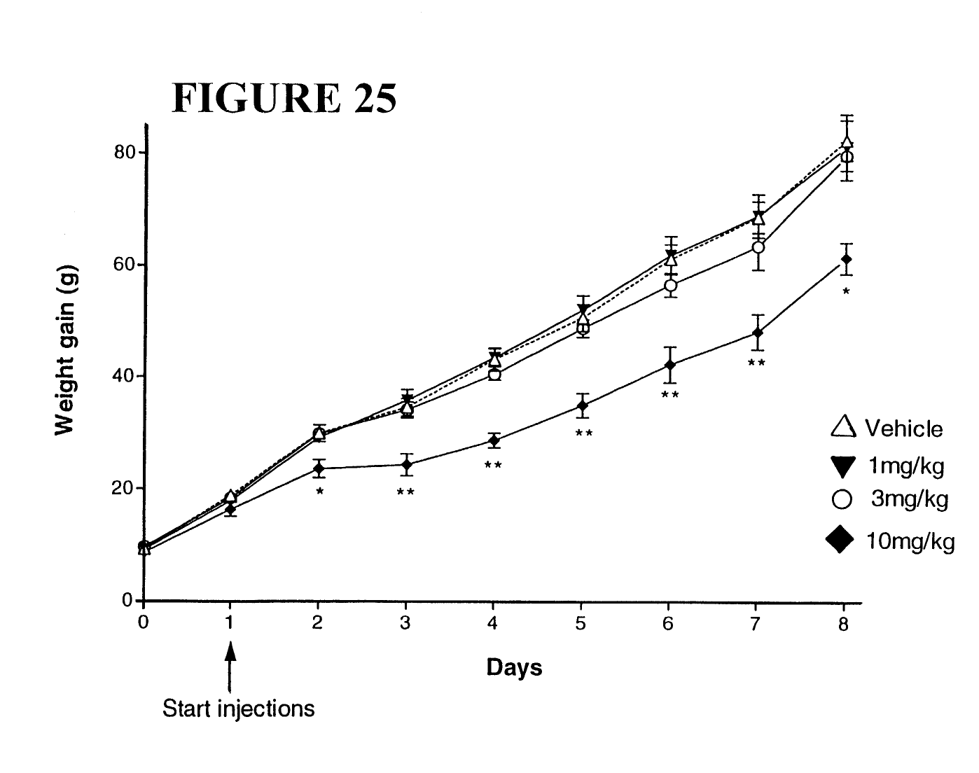

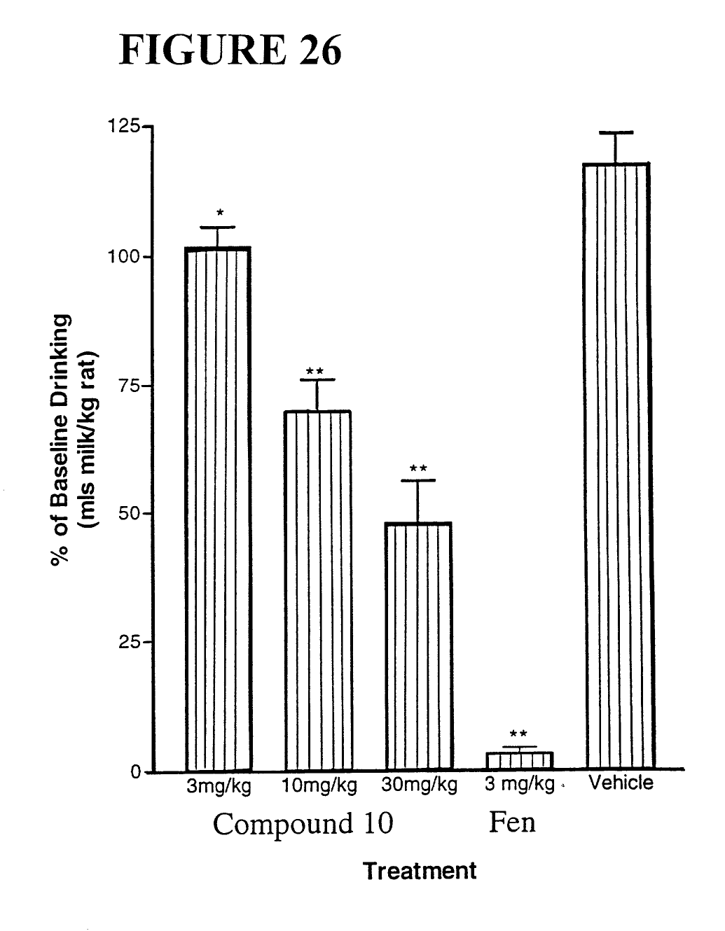

A low density of MCH1 receptor binding sites was detected in the cerebral cortex with slightly increased binding in the superficial layers. The septal nuclei (FIGS. 20A, C), claustrum (Cl) (FIGS. 20A, B), ventral and horizontal limbs of the diagonal band, and piriform cortex (Pir) likewise contained a low density of MCH1 receptor binding sites (FIGS. 20A, A-F; 20B, G and H).

Some of the highest MCH1 receptor binding in the rat CNS was observed in the basal ganglia and the olfactory tubercle (Tu) (FIGS. 20A, B). The caudate-putamen (CPu) and core of the accumbens nucleus (AcbC) displayed dense labeling of MCH1 receptors while a very intense labeling was present in the shell of the accumbens nucleus (Acbsh) (FIGS. 20A, B). The globus pallidus (GP) was unlabeled. The subthalamic nuclei (STh), part of the basal ganglia circuit, was moderately labeled (FIGS. 20A, F).

The amygdala and extended amygdala displayed a moderately low labeling with slightly higher radioligand binding observed in the bed nucleus of the stria terminalis (BSTM) (FIGS. 20A, C), the basolateral (BLA) and lateral amygdaloid nuclei (LA) (FIGS. 20A, D and F).

Diencephalon

In general, MCH1 receptor binding was weak throughout the diencephalon. In the thalamus there was a slight increase in binding intensity in the paraventricular (PVA), centromedial, and anterodorsal thalamic nuclei (AD) (FIGS. 20A, D). In the epithalamus the medial habenular nucleus (MHb) contained MCH1 receptor binding sites (FIGS. 20B, G). Throughout the hypothalamus there was a uniformly weak binding signal (FIGS. 20A, C-H; 20B, G and H). There was a slight increase in MCH1 binding intensity in the ventromedial hypothalmus (VMH) and in the medial mammillary nucleus (MM) (FIGS. 20A, E; 20B H).

MCH1 receptor binding was moderate in the induseum griseum (IG) (FIGS. 20A, B) and in Ammon′ s horn of the hippocampal formation (CA1, CA2, CA3) (FIGS. 20A, E and F). MCH1 binding sites were present in the stratum oriens (so) and stratum radiatum (sr) of field CA1, and in the stratum oriens of field CA3. Moderate binding was observed in the molecular layer of the dentate gyrus and in the pre/parasubiculum (FIGS. 20A, B and H).

Mesencephalon

Overall, MCH1 receptor binding in the mesencephalon was very weak. A slight increase in binding intensity was evident in the periaqueductal gray (PAG) and in the pontine nuclei (Pn) (FIGS. 20B, I and J). Moderate binding was observed in the superior colliculus (SC) and the dorsal raphe nucleus (DR) (FIGS. 20B, I and J).

Rhombencephalon (Pons/Medulla)

The highest density of MCH1 receptor binding sites in the rhombencephalon was seen in the locus coeruleus (LC) (FIGS. 20B, L). There was consistently low MCH1 receptor binding throughout the pons and medulla (FIGS. 20B, K and L). Slightly higher binding was detected in the inferior colliculus (IC), the dorsal tegmental nuclei (DTN) and parabrachial nuclei (PB) (FIGS. 20B, K) and the lateral superior olive (LSO) (FIGS. 20B, L).

Spinal Cord

MCH1 receptor binding sites appeared to be uniformly distributed throughout the dorsal and ventral horns of the spinal cord (FIGS. 20B, M). Binding density was slightly increased in the superficial dorsal horn.

Table 4

The distribution of MCH1 receptor binding sites in the rat CNS using Receptor Autoradiography with 0.1 nM [³H]Compound 10 in the presence of 1 μM prazosin and 100 μM dopamine. The strength of [³H]Compound 10 (MCH1) labeling intensity for the various rat brain regions was graded as absent (−), weak (+) moderate (++), heavy (+++), or intense (++++).

| | Density of | |
|---|---|---|
| | MCH1 receptor | Potential |
| Region | binding sites | Application |
| Olfactory System | | Modulation of |
| | | olfactory |
| | | sensation |
| Anterior olfactory n. | + | |
| Olfactory tubercle | +++ | |
| Islands of Calleja, | +++ | |
| major | | |
| Telencephalon | | Cognition |
| Claustrum | ++ | Visual |
| | | attention |
| Dorsal endopiriform n. | + | Olfactory |
| | | information |
| | | processing |
| Basal Ganglia | | |
| Globus pallidus | − | |
| Caudate-putamen | +++ | Sensory/motor |
| | | integration |
| Accumbens n., shell | ++++ | Treatment of |
| | | drug addiction. |
| | | This region is |
| | | particularly |
| | | sensitive to |
| | | psychoactive |
| | | drugs. |
| Accumbens n., core | +++ | Treatment of |
| | | schizophrenia, |
| | | anxiety and/or |
| | | depression. |
| Medial septal n. | + | Cognitive |
| | | enhancement via |
| | | cholinergic |
| | | system |
| Septohippocampal n. | + | |
| Amygdala | | Modulation of |
| | | endocrine |
| | | functions and |
| | | integrated |
| | | behaviors such |
| | | as defense, |
| | | ingestion, |
| | | reproduction, |
| | | and learning. |
| | | Treatment of |
| | | anxiety and/or |
| | | depression. |
| Central amygdaloid n. | + | Fear and |
| | | anxiety |
| Basolateral amygdaloid | + | Olfaction |
| n. | | |
| Bed n. Of the stria | ++ | Modulation of |
| terminalis | | the limbic |
| | | system. |
| | | Treatment of |
| | | anxiety and/or |
| | | depression. |
| Anterior cortical n. | + | Olfaction |
| Diencephalon | | |
| Thalamus | | Analgesia/ |
| | | Modulation of |
| | | sensory |
| | | information |
| Paraventricular n. | + | Modulation of |
| | | motor and |
| | | behavioral |
| | | responses to |
| | | pain |
| Centromedial n. | + | Modulation of |
| | | motor and |
| | | behavioral |
| | | responses to |
| | | pain |
| Anterodorsal n. | + | Modulation of |
| | | motor |
| | | information to |
| | | the cerebral |
| | | cortex/eye |
| | | movement |
| Reticular n. | + | Alertness/Seda- |
| | | tion |
| Mediodorsal n. | − | |
| Hypothalamus | + | Regulation of |
| | | endocrine |
| | | function, |
| | | reproductive |
| | | behaviors, and |
| | | appetite/obesity. |
| | | Treatment of |
| | | anxiety and/or |
| | | depression. |
| Hippocampal formation | | Cognition/memory |
| | | consolidation |
| | | and retention |
| CA1 | ++ | |
| CA2 | ++ | |
| CA3 | ++ | |
| pre/parasubiculum | ++ | Modulation of |
| | | memory |
| | | aquisition |
| Mesencephalon | | |
| Superior colliculus | ++ | Modulation of |
| | | visual |
| | | information/ |
| | | spatial |
| | | localization |
| Pontine n. | + | |
| Periaqueductal gray | + | Analgesia |
| Substantia nigra | + | |
| interpeduncular n. | ++ | Analgesia |
| Caudal linear raphe n. | + | |
| Locus coeruleus | ++ | Modulation of |
| | | NA transmission |
| Cerebellum | − | |
| Spinal cord | | |
| Dorsal horn | + | Nociception/ |
| | | Analgesia |
| Ventral horn | + | Spinal reflex |

Discussion

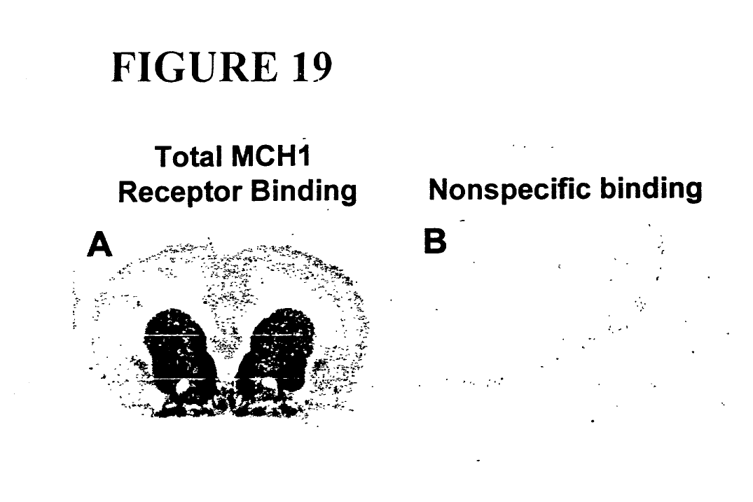

The anatomical distribution of the MCH1 receptor in the rat CNS was determined by receptor autoradiography using [³H]Compound 10 at 0.1 nM in the presence of 100 μM dopamine and 1 μM prazosin to directly visualize the receptor (FIG. 19 A). Nonspecific binding was determined by including 10 μM unlabeled Compound 10 in the incubation buffer. The specific binding of [³H]Compound 10 was approximately 95% (FIG. 19 B).

The results suggest that the MCH1 receptor is widely distributed in the rat CNS. MCH1 receptors are abundantly expressed in the basal ganglia and moderately expressed in the hippocampus and locus coeruleus. Weak MCH1 expression was observed throughout the diencephalon, mesencephalon and rhombencephalon. The spinal cord exhibited low expression of the MCH1 receptor in the dorsal and ventral horns.

MCH-like immunoreactivity (MCH-LI) has been described in the rat CNS (Skofitsch, G. et al. 1985; Zamir, et al., 1986; Bittencourt et al. 1992). MCH-LI was detected throughout the entire brain, including the neocortex, striatum, amygdala, hippocampus, diencephalon, mesencephalon, and mylencephalon. Only the cerebellar cortex did not contain MCH-LI. MCH cell bodies were located in the hypothalamus, in the olfactory bulb spreading caudally to the anterior amygdaloid area, and in the region of the paramedian pontine reticular formation. The diencephalon contained the highest concentration of MCH-positive cell bodies and an extensive fiber network. Telencephalic areas received a dense MCH immunoreactive fiber network. Sparse MCH positive fibers were seen in the neocortex, hippocampus, olfactory tubercle, caudate-putamen, nucleus accumbens, thalamus,and the medulla and the spinal cord.

Recently with the cloning of the MCH1 receptor (SLC-1) (Saito, et al., 1999; Chambers, et al., 1999) the tissue localization for MCH1 mRNA has been revealed. MCH1 mRNA was localized to a variety of brain regions involved including olfactory regions, the hippocampus, basal ganglia, hypothalamus, amygdala, and locus coeruleus. There was a particularly robust expression of mRNA in the accumbens nucleus which is involved in behavioral reinforcement. Subsequently, using receptor selective antibodies the MCH1 (SLC-1) receptor protein distribution was found to concordant with the distribution of the MCH1 mRNA in the rat CNS (Hervieu, et al., 2000). The distribution of MCH1 binding sites using [³H] Compound 10 herein reported parallels the distribution of both receptor mRNA and protein expression. The extensive distribution of MCH1 receptor binding sites throughout the rat CNS is not surprising because MCH cells in the lateral hypothalamus and zona incerta project widely throughout the brain.

Potential Application

MCH has been associated with regulation of food intake and feeding behavior (Qu, et al., 1996; Rossi, et al., 1997; Shimada, et al., 1998), the control of goal oriented behaviors, general arousal or stress responses (Jezova, et al.,1992) and the regulation of fluid homeostasis (for review, Bernardis, et al. 1993).

The anatomical distribution of MCH1 receptor binding sites is consistent with a role for the MCH receptor in the regulation of food intake, thirst, and the reinforcement of feeding behaviors. MCH1 receptor binding sites were evident in the ventromedial, dorsomedial, and arcuate nuclei which are areas that are recognized to be involved in food intake, suggesting that the MCH1 receptor mediates the orexigenic effects of MCH. MCH1 binding sites were present in regions involving the regulation of fluid homeostasis, the lateral hypothalamus and the zona incerta.

As already stated, the MCH1 binding sites are widely distributed throughout the brain. The extensive localization of MCH1 receptors in the neocortex and the lateral hypothalamus supports a functional role for the MCH1 receptor in general arousal.

MCH has been shown to increase ACTH release in vivo and to have a stimulatory effect on the hypothalamic-pituitary-adrenal gland axis (HPA). The site of action of MCH is currently unknown, however one possible target are CRF neurones located throughout the hypothalamus and the bed nucleus of the stria terminalis. MCH1 receptors have been localized to these regions thus supporting a potential role for the MCH1 receptor in the stress response.

MCH1 receptors are present in several limbic system-related structures, namely the hippocampus, septum, accumbens nucleus, nucleus of the diagonal band, bed nucleus of the stria terminalis, and the amygdala. On the basis of this localization, the MCH1 receptor may be involved in the regulatation of learning and memory as well as emotional states. It has been established that the drugs that are effective in the treatment of depression and anxiety primarily act on the serotonergic and noradrenergic systems in the brain. MCH1 receptors have been localized in several forebrain areas that receive projections from midbrain raphe nuclei, the origen of the serotonergic pathway, as well as the locus coeruleus where the noradrenergic system originates. MCH1 receptors in the amygdala, hippocampus, hypothalamus, accumbens nucleus and the neocortex may be targets for the treatment of mood disorders.

The most impressive radioligand binding in the rat CNS was in the basal ganglia, specifically the caudate-putamen, and accumbens nucleus, with moderate binding in the subthalamic nucleus. Taken together with MCH1 receptor localization throughout the motor cortex and the reticular formation, regions associated with locomotion activity, MCH1 receptors may potentially mediate MCH's role in controlling motor behavior and thus may be potentail therapeutic target in the treatment of Parkinson's disease and Huntington's Chorea. It is however, noteworthy that there were no locomotion deficits found in the open-field locomotion test on MCH^{-/-} mice.

The localization of MCH1 receptor in the ventral striatum is rather interesting and suggests that the MCH1 receptor is a possible therapeutic target in the treatment of drug addiction and psychosis via the regulation of dopaminergic neurotransmission. The accumbens nucleus is involved in the mediation of positive reinforcement of feeding behavior and plays a role in reward mechanisms. There is a dense dopaminergic projection from the ventral tegmental area to the accumbens nucleus which is the site of action of antipsychotic drugs.

A role for the MCH1 receptor in regulating sensory information might be indicated by their presence in the relay nuclei of several sensory pathways. It appears that the MCH1 receptor may participate in the modulation of the visual system. MCH1 receptor binding sites are localized to the superior colliculus which receives afferents from the retina. In the auditory system the MCH1 receptor is present in the medial geniculate, inferior colliculus, and the cochlear and medial vestibular nuclei.

The localization of MCH1 receptors in the locus coeruleus implies a potential modulatory action in noradrenergic neurotransmission, influencing sleep, attention and vigilance.

A potential role for the MCH1 receptor in the modulation of the perception of pain is supported by the localization of MCH1 receptor binding sites in the periaqueductal gray, dorsal raphe nucleus and in the gray matter of the spinal cord. MCH1 receptors are in a position to modulate incoming as well as descending sensory information and also spinal motor reflexes.

In-Vivo Models

Results

1. Effects on MCH-Stimulated Food Intake

The administration of MCH into the third ventricle significantly increased food intake at all time points measured compared to vehicle treated controls (FIG. 21). Significant differences in food intake were observed among conditions after 30 [F(4,44)=7.07, p<0.0002, one-way repeated measure ANOVA], 60 [F(4,44)=6.31, p<0.0004] and 120 [F(4,44)=9.84, p<0.0001] min. Pretreatment with systemic DMSO prior to MCH did not significantly alter the MCH-induced food intake (FIG. 21). Pretreatment with 10 mg/kg of Compound 10 significantly reduced the magnitude of MCH-induced feeding after 30 and 120 minutes and prevented the occurrence of significant MCH-induced feeding at 60 minutes (FIG. 21). Pretreatment with 1 mg/kg of Compound 10 prevented the occurrence of significant MCH-induced feeding over the time period (FIG. 21). These results demonstrate that the peripheral administration of Compound 10 significantly attenuates the stimulation of food intake induced by the central administration of MCH.

2. Effects of MCH1 Antagonists on Body Weight

To test the effect of Compound 10 on body weight regulation, we monitored body weight in rats implanted with osmotic minipumps which released either 10 mg/kg/day of Compound 10, 6 mg/kg/day of d-fenfluramine or vehicle. Rats treated with either Compound 10 or d-fenfluramine gained significantly less weight than vehicle-treated rats [by 2-way ANOVA, F(2,360)=81.69, p<0.0001] . Rats treated with Compound 10 gained significantly less weight than vehicle-treated controls on days 1-13 (FIG. 22). Rats treated with d-fenfluramine gained significantly less weight than vehicle-treated controls on days 1-6 (FIG. 22). Over the duration of the study, rats treated with Compound 10 or d-fenfluramine gained 16% and 10% less, respectively, from controls.

The effect of Compound 10 on body weight was next tested by administering it to rats twice a day for 7 days by i.p. injection. As shown in FIG. 23, while all rats gained weight over the course of the study, rats treated with 10 mg/kg of Compound 10 gained significantly less weight than vehicle-treated controls [effect of treatment by two-way ANOVA: F(3,214)=60.59, P<0.0001]. Rats treated with 10 mg/kg of Compound 10 gained 26% less weight compared to vehicle-treated rats. There was no significant effect on body weight from the administration of 1 or 3 mg/kg of Compound 10.

The effects of Compound 94 and Compound 95 on body weight were tested by twice daily i.p. administration for 3 days. By 2-way ANOVA, there was a significant effect of treatment on body weight gain [F(6,160)=31.21, p<0.0001, FIGS. 24, 5). As shown in FIG. 24, rats treated with 30 mg/kg of Compound 94 gained significantly less weight than vehicle-treated controls on days 1, 2 and 3, while rats treated with 3 or 10 mg/kg of Compound 94 gained weight similarly to controls. Similarly, as shown in FIG. 25, rats treated with 30 mg/kg of Compound 95 gained significantly less weight than vehicle-treated controls on days 1, 2 and 3, while rats treated with 3 or 10 mg/kg of Compound 95 gained weight similarly to controls. Over the duration of the study, rats treated with Compound 94 or Compound 95 gained 40% and 82% less, respectively, from controls. Taken together, these findings demonstrate that three compounds which are antagonists at the MCH1 receptor produce a decrease in body weight gain in young, growing rats.

3. Effects of MCH1 Antagonists on Consumption of Sweetened Condensed Milk

Rats were trained to drink sweetened condensed milk during 7 daily 20 min sessions in the early part of the light cycle. Baseline drinking for each animal was determined as the average of the last three days of training. Rats were then exposed to sweetened condensed milk 30 minutes following i.p. administration of either vehicle, 3, 10 or 30 mg/kg of Compound 10 or 3 mg/kg of d-fenfluramine. FIG. 26 shows the amount of milk consumed, expressed as a percentage of each animals' baseline drinking. Rats treated with 3, 10 or 30 mg/kg of Compound 10 or 3 mg/kg of d-fenfluramine drank significantly less than vehicle-treated rats (87, 59, 41 and 0.03% less, respectively). These results suggest that Compound 10 acts as an anorectic agent and is capable of decreasing consumption of a palatable food.

4. Forced Swim Test

The Effect of Vehicle, Fluoxetine and Compound 10 on Immobility, Climbing and Swimming in the Forced Swim Test

Immobility

Statistical analysis indicated that there was a significant drug effect [F(4,24)=6.36, p=0.0018] on immobility. Subsequent post hoc analysis revealed that a single injection of 10 mg/kg i.p. of fluoxetine significantly decreased immobility to 23.0+1.0 (Student-Newman-Keuls value was 20.52, p<0.01) compared to vehicle-treated controls (Table 5). In addition, a single injection of either 3, 10 or 30 mg/kg i.p. of Compound 10 significantly decreased immobility (25±2.7, 25±1.9 & 26±1.9 counts at each dose, respectively) compared to vehicle-treated controls 35+1.9 (Student-Newman-Keuls values of 13.45, 15.08 and 11.91, respectively; Table 5).

Climbing

The statistical analysis of the climbing counts indicated that there was a significant drug effect [F(4,24)=5.18, p=0.005] . Post hoc analysis indicated that a single injection of 10 mg/kg of fluoxetine did not significantly alter climbing counts compared to vehicle-treated animals (Table 5). In contrast, a single injection of 3 mg/kg of Compound 10 produced a significant increase (19±1.7) in climbing counts (Student-Newman-Keuls value=9.42, p<0.05) compared to vehicle-treated animals (12±1.2). Compound 10 dosed at 10 & 30 mg/kg did not significantly alter climbing.

Swimming

The statistical analysis of the swimming data indicated that there was a significant drug effect [F(4,24)=16.4, p<0.0001] (Table 5). The post hoc test showed that a single injection of 10 mg/kg i.p. of fluoxetine produced a significant increase (26+1.6) in swimming counts over the vehicle treated animals (12 i 1; Student-Newman-Keuls value of 50.48, p<0.01). Similarly, a single injection of 3, 10 or 30 mg/kg i.p. of Compound 10 significantly increased swimming counts (16±1.1, 20±1.7 & 23±1.0, respectively; Student-Newman-Keuls values of 4.37, p<0.05, 19.26, p<0.01; 34.2, p<0.01; Table 5).

Diving

This behavior was not observed following a single injection of vehicle and rarely observed following fluoxetine (1 animal out of 5 dove twice), 3 mg/kg of Compound 10 (2 animals out of 5 dove once each), 10 mg/kg of Compound 10 (4 animals out of 5 had counts of 1, 4, 1 and 3) or 30 mg/kg of Compound 10 (2 rats out of 5 had counts of 1 and 4).

TABLE 5
The effect of vehicle, Compound 10 and
fluoxetine on immobility, climbing and swimming in the
rat forced swim test.

| Treatment | Dose (mg/kg) | Immobility | Climbing | Swimming |
|---|---|---|---|---|
| Vehicle | — | 35 ± 1.9^{c} | 12 ± 1.2 | 12 ± 1.0^{a} |
| Compound 10 | 3 | 25 ± 2.7 | 19 ± 1.7^{b} | 16 ± 1.1 |
| Compound 10 | 10 | 25 ± 1.9 | 13 ± 1.7 | 20 ± 1.7 |
| Compound 10 | 30 | 26 ± 1.9 | 10 ± 1.8 | 23 ± 1.2 |
| Fluoxetine | 10 | 23 ± 1.0 | 12 ± 0.8 | 26 ± 1.6 |

Each value represents the mean ± S.E.M.
A total of 5 animals were examined for each treatment group.
^{a}significantly less than 3 mg/kg Compound 10 (p < 0.05) and 10 and 30 mg/kg Compound 10 and fluoxetine, ANOVA and Student-Newman-Keuls test.
^{b}Significantly greater than Vehicle (p < 0.05) and 10 and 30 mg/kg Compound 10 (p < 0.01), ANOVA and Student-Newman-Keuls test.
^{c}Significantly greater than 3, 10 and 30 mg/kg Compound 10 and fluoxetine, p < 0.01, ANOVA and Student-Newman-Keuls test

The results of the Forced Swim Test indicate that using a modified version of the Porsolt forced swim test, a single injection of 10 mg/kg i.p. of fluoxetine produced a significant decrease in immobility and an increase in swimming in male Sprague-Dawley rats. This is consistent with findings from previous studies using the Lucki version (Detke, et al., 1995; Kirby and Lucki, 1997; Lucki, 1997; Page, et al., 1999; Reneric and Lucki, 1998). In addition, the results obtained using fluoxetine are consistent with those using other SSRIs (Detke, et al., 1995). Thus, a modified version of the Porsolt forced swim test can consistently detect the antidepressant action of SSRIs such as fluoxetine.

Compared to vehicle-treated animals, Compound 10 produced a significant decrease in immobility and a significant increase in swimming at doses of 3, 10 and 30 mg/kg, and a significant increase in climbing at 3 mg/kg. Thus, based on past interpretations of the Forced Swim Test, our results suggest that Compound 10 has antidepressant-like properties.

5. Social Interaction Test

The Effect of Compound 10 and Chlordiazepoxide on Behavior in the Rat Social Interaction Test

A single i.p. administration of either 3, 10 or 30 mg/kg of Compound 10 significantly increased social interaction (Student-Newman-Keuls values of 48.7, 43.7 & 17.1, respectively), as did the benzodiazepine anxiolytic, chlordiazepoxide (Student-Newman-Keuls value of 58.8) compared to vehicle-treated animals [ANOVA, F(4,40)=20.6, p<0.0001; Table 6). The degree of social interaction produced by the 30 mg/kg i.p. dose of Compound 10 was significantly less than that of 3 and 10 mg/kg of Compound 10 and 5 mg/kg of chlordiazepoxide (Student-Newman-Keuls values of 8.06, 8.12 and 14.16, respectively). There was no significant difference in the duration of social interaction between 3 and 10 mg/kg i.p. of Compound 10 and chlordiazepoxide.

TABLE 6
The Effect Of A Single Injection Of Vehicle,
Chlordiazepoxide And Compound 10 On The Social Interaction
And Rearing Of Unfamiliar Cage Mates In A Familiar Arena

| Drug Treatment | Dose | Social Interaction (sec) |
|---|---|---|
| Vehicle | — | 102 ± 6.1^{A} |
| Chlordiazepoxide | 5 mg/kg | 228 ± 11^{*} |
| Compound 10 | 3 mg/kg | 210 ± 14^{*} |
| Compound 10 | 10 mg/kg | 215 ± 15^{*} |
| Compound 10 | 30 mg/kg | 166 ± 11*^{&} |

^{A}Each value represents the mean seconds of social interaction ± S.E.M.
A total of 7-10 animals were examined for each treatment group.
*Significantly greater than Vehicle, p < 0.01, ANOVA and Student-Newman-Keuls test.
^{&}Significantly less than chlordiazepoxide, 3 mg/kg of Compound 10 (p < 0.01) and 10 mg/kg of Compound 10 (p < 0.05), ANOVA and Student-Newman-Keuls test.

The Effect of Compound 10 and Chlordiazepoxide on Rearing Behavior, Locomotor Activity and Grooming in the Rat Social Interaction Test

Statistical analysis indicated a significant effect of treatment on rearing behavior (ANOVA, F(4,40)=3.03, p=0.028, Table 7). A single i.p. administration of 3 mg/kg of Compound 10 significantly lowered the number of rearings displayed compared to animals treated with vehicle and 10 or 30 mg/kg of Compound 10 (Student-Newman-Keuls values of 4.55, 5.34 and 8.09, respectively). In addition, the number of rearings produced by 5 mg/kg i.p. of chlordiazepoxide was significantly lower than that for the 30 mg/kg dose of Compound 10 (Table 7).

Statistical analysis indicated a significant effect of treatment on grooming behavior (F(4,40)=12.00, p<0.0001; Table 7). A single i.p. administration of 30 mg/kg of Compound 10 produced a significantly greater number of grooming bouts compared to animals treated with vehicle, 3 or 10mg/kg of Compound 10 and 5 mg/kg of chlordiazepoxide (Student-Newman-Keuls values of 16.9, 25,1, 27.9 and 36.6, respectively). There was no significant difference in the number of grooming bouts between the 3 and 10 mg/kg doses of SNEC-3 and vehicle-treated animals.

Statistical analysis indicated a significant effect of treatment on locomotor activity (F(4,40)=3.93, p=0.0088). Post hoc analyses indicated that the number of squares crossed following the 3 mg/kg dose of Compound 10 was significantly lower than animals treated with either vehicle, 10 or 30 mg/kg of Compound 10 (Student-Newman-Keuls values of 8.4, 6.5 and 8.96, respectively, Table 7). There was no significant difference in the number of squares crossed between animals treated with a single injection of 5 mg/kg of chlordiazepoxide and the other treatment groups.

TABLE 7
The effect of a single injection of vehicle,
chlordiazepoxide and Compound 10 on the number of
rearings, grooming episodes and squares crossed in the
social interaction test.

| Drug | | | Squares | Grooming |
|---|---|---|---|---|
| Treatment | Dose | Rearing | Crossed | Bouts |
| Vehicle | — | 56 ± 6 | 484 ± 52 | 6.2 ± 0.7 |
| Chlordiazepoxide | 5 mg/kg | 46 ± 5 | 355 ± 49 | 3.6 ± 0.7 |
| Compound 10 | 3 mg/kg | 43 ± 4^{a} | 327 ± 24^{c} | 5.2 ± 0.5 |
| Compound 10 | 10 | 59 ± 3 | 480 ± 46 | 4.3 ± 0.4 |
| | mg/kg | | | |
| Compound 10 | 30 | 61 ± 4^{b} | 490 ± 29 | 10.8 ± 1.3^{d} |
| | mg/kg | | | |

All values represent the mean ± S.E.M. A total of 7-10 animals were examined for each treatment group.
^{a}Significantly less than vehicle, 10 and 30 mg/kg Compound 10, p < 0.05, ANOVA and Student-Newman-Keuls test.
^{b}Significantly greater than chlordiazepoxide (p < 0.05) and 3 mg/kg of Compound 10 (p < 0.01), ANOVA and Student-Newman-Keuls test
^{c}Significantly less than vehicle, 3 and 30 mg/kg Compound 10 (p < 0.05), ANOVA and Student-Newman-Keuls test.
^{d}Significantly greater than all other treatment groups, p < 0.01, ANOVA and Student-Newman-Keuls

At doses of 3, 10 and 30 mg/kg i.p., Compound 10 produced a significant increase in social interaction time in male rats compared to vehicle-treated animals. Also, the anxiolytic agent (5 mg/kg i.p. chlordiazepoxide) produced a significant increase in social interaction time compared to vehicle-treated animals. The response produced by either the 3 or 10 mg/kg doses of Compound 10 was comparable to that of the positive control, chlordiazepoxide. The degree of social interaction produced by the 30 mg/kg i.p. dose of Compound 10 was significantly lower than that for the 3 and 10 mg/kg doses. Since this higher dose of Compound 10 resulted in a significant increase in grooming behavior compared to other treatment groups, it is possible that it was producing behaviors that were competing with the social interaction time.

A single administration of 3 mg/kg of Compound 10 produced a significant decrease in the number of squares crossed and rearing episodes compared to animals treated with vehicle and 10 or 30 mg/kg of Compound 10. The behavioral significance of this finding is unknown. However, the animals treated with the 3 mg/kg dose of Compound 10 did not show any obvious signs of catalepsy or sedation. In addition, the level of social interaction at this dose was not significantly different from that of 10 mg/kg of Compound 10 or 5 mg/kg of chlordiazepoxide.

Previously, it has been shown that in the social interaction test, analeptics such as amphetamine and caffeine, increase social interaction and locomotor activity, whereas anxiolytics increase social interaction time but actually decrease locomotor activity (File, 1985; File and Hyde, 1979; Guy and Gardner, 1985). However, none of the doses of Compound 10 significantly increased either rearing behavior or squares crossed compared to vehicle-treated animals. In fact, as stated above, the number of squares crossed and rearing behavior was significantly reduced at the 3 mg/kg i.p. dose of Compound 10 compared to vehicle-treated animals. Thus, it is unlikely that Compound 10 is producing a non-specific effect.

In conclusion, the results of this study indicate that Compound 10, at doses of 3, 10 and 30 mg/kg i.p. , significantly increased social interaction time without producing a significant increase in squares crossed or rearing behavior. Overall, Compound 10 appears to have the profile of an anxiolytic drug in the social interaction test.

### REFERENCES

Abrao, M. S., Castrucci, A. M., Hadley, M. E. and Hruby, V. J. (1991) Protein-kinase-C mediates MCH signal transduction in teleost, Synbranchus marmoratus, melanocytes. Pigment. Cell. Res. 4:66-67.

Auburger, G., Gispert, S., Scheufler, K., Nothers, C., Lunkes, A., Hernandez, A., Magarino, C., Enczmann, J., Freund, H. J., Heredero, L., and Orozco, G. (1992) Assignment of the second (cuban) locus of autosomal dominant cerebellar ataxia to chromosome 12q23-24.1, between flanking markers D12S58 and PLA2. Cytogenet. Cell. Genet. 61:252-256.

Bahjaoui-Bouhaddi, M., Fellmann, D., Griffond, B. and Bugnon, C. (1994) Insulin treatment stimulates the rat melanin-concentrating hormone-producing neurons. Neuropeptides 24:251-258.

Bakker, R. A., et al., (2000) Constitutive activity of the histamine Hi receptor reveals inverse agonism of histamine Hi receptor antagonists. Eur. J. Pharmacol., 387: R5-R7.

Baker, B. I. (1994) Melanin-concentrating hormone update: functional consideration. TEM 5:120-126.

Baker, B. I. (1991) Melanin-concentrating hormone: a general vertebrate neuropeptide. Int. Rev. Cytol. 126:1-47.

Bassett, A. S., Jones, B. D., McGillivray, B. C. and Pantzer, J. T. (1988) Partial trisomy chromosome 5 cosegregating with schizophrenia. Lancet 1:799-801.

Bernardis, L. I. and Berlinger, L. L. (1993) The lateral hypothalamic area revisited: neuroanatomy, body weight regulation, neuroendocrinology and metabolism. Neurosci. Biobehav. Rev. 17:141-193.

Bittencourt, J. C., et al., (1992) The melanin-concentrating hormone system of the rat brain: An immuno- and hybridization histochemical characterization. J. Comp. Neurol. 319:218-245.

Bradford, M. M. (1976) A rapid and sensitive method for the quantitation of microgram quantities of protein utilizing the principle of protein-dye binding. Anal Biochem 1976 May 7; 72:248-54.

Breton, C., Schorpp, M., and Nahon, J. L. (1993) Isolation and characterization of the human melanin-concentrating hormone gene and a variant gene. Mol. Brain Res. 18:297-310.

Burgaud, J. L., Poosti, R., Fehrentz, J. A., Martinez, J., and Nahon, J. L. (1997) Melanin-concentrating hormone binding sites in human SVK14 keratinocytes. Biochem.Biophys.Res.Commun. 241(3):622-629.

Burns, C. C., Moser, M., Banks, J., Alderete, J. P., and Overbaugh, J. (1996) Identification and deletion of sequences required for feline leukemia virus RNA packaging and construction of a high-titer feline leukemia virus packaging cell line. Virology (Aug. 1, 1996) 222(1):14-20.

Chambers, J., et. al. (1999) Melanin-concentrating hormone is the cognate ligand for the orphan G-protein-coupled receptor SLC-1. Nature 444:216-265.

Chu, Y. Y., Tu, K. H., Lee, Y. C., Kuo, Z. J., Lai, H. L., and Chern, Y. (1996) Characterization of the rat A2a adenosine receptor gene. DNA Cell Biol (1996 April) 15(4):329-37.

Coleman, A. (1984) Transcription and Translation: A Practical Approach (B. D. Hanes, S. J. Higgins, eds., pp 271-302, IRL Press, Oxford, 1984).

Craddock, N., Dawson, E., Burge, S., Parfitt, L., Mant, B., Roberts, Q., Daniels, J., Gill, M., McGuffin, P., Powell, J. and Owen, M. (1993) The gene for Darier's disease maps to chromosome 12q23-q24.1. Hum. Mol. Genet. 2:1941-1943.

Cullen, B. (1987) Use of eukaryotic expression technology in the functional analysis of cloned genes. Methods Enzymol., 152: 685-704.

Dascal, N., Schreibmayer, W., Lim, N. F., Wang, W., Chavkin, C., DiMagno, L., Labarca, C., Kieffer, B. L., Gaveriaux-Ruff, C., Trollinger, D., Lester, H. A., Davidson, N. (1993) Atrial G protein-activated K+ channel: expression cloning and molecular properties. Proc. Natl. Acad. Sci. USA 90:10235-10239.

deLigt, R. A., et al., (2000) Inverse agonism at G protein-coupled receptors: (patho)physiological relevance and implications for drug discovery. Br. J. Pharmacol., 130(1): 1-12.

Detke, M. J., et al., (1995) Active behaviors in the rat forced swim test differentially produced by serotonergic and noradrenergic antidepressants. Psychopharmacology, 121: 66-72.

Dondoni, A., et al. T. Synthesis (1995), 181.

Drozdz, R. and Eberle, A. N. (1995) Binding sites for melanin-concentrating hormone (MCH) in brain synaptosomes and membranes from peripheral tissues identified with highly tritiated MCH. J.Recept.Signal.Transduct.Res. 15(1-4):487-502.

Drozdz, R., Siegrist, W., Baker, B. I., Chluba-de Tapia, J. and Eberle, A. N. (1995) Melanin-concentrating hormone binding to mouse melanoma cells in vitro. FEBS 359:199-202.

Drozdz, R., Hintermann, E., and Eberle, A. N. (1998) Characterization of the receptor for melanin-concentrating hormone on melanoma cells by photocrosslinking. Ann.NY Acad.Sci. 839(1):210-213.

Elmquist, J. K., et al.,(1999) From lesions to leptin: hypothalamic control of food intake and body weight. Neuron 22:221-232.

Fan, W., et al.,(1997)Role of melanocortinergic neurons in feeding and the agouti obesity syndrome. Nature 385:165-168.

File, S. E. (1985) Animal models for predicting clinical efficacy of anxiolytic drugs: social behaviour. Neuropsychobiology, 13: 55-62.

File, S. E. and Pellow, S. (1984) The anxiogenic action of FG 7142 in the social interaction test is reversed by chlordiazepoxide and Ro-15-1788 but not by CGS 8216. Archs. Int. Pharmacodyn. Ther., 271: 198-205.

File, S. E. and Pellow, S. (1983) The anxiogenic action of a convulsant benzodiazepine: reversal by chlordiazepoxide. Brain Res., 278: 370-372.

File, S. E., et al., (1982) The anxiogenic action of benzodiazepine-like antagonists. Neuropharmacology, 21: 1033-1037.

File, S. E. (1980) The use of social interaction as a method for detecting anxiolytic activity of chlordiazepoxide-like drugs. J. Neurosci. Methods, 2: 219-238.

File, S. E. and Hyde, J. R. G. (1979) A test of anxiety that distinguishes between the actions of benzodiazepines and those of other minor tranquilisers and of stimulants. Pharmacol. Behav. Biochem., 11: 65-69.

File, S. E. and Hyde, J. R. G. (1978) Can social interaction be used to measure anxiety? Br. J. Pharmacol., 62: 19-24.

Fong, T. M.; Huang, R. C.; Yu, H.; Swain, C. J.; Underwood, D.; Cascieri, M. A.; Strader, C. D. (1995) Mutational analysis of neurokinin receptor function. Can. J. Physiol. Pharmacol. 73(7):860-865 (July 1995).

Gantz, I, et al.,(1993) Molecular cloning, expression, and gene localization of a fourth melanocortin receptor. J Biol Chem. 268:15174-15179.

Gilliam, T. C., Freimer, N. B., Kaufmann, C. A., Powchik, P. P., Bassett, A. S., Bengtsson, U. and Wasmuth, J. J. (1989) Deletion mapping of DNA markers to a region of chromosome 5 that cosegregates with schizophrenia. Genomics 5:940-944.

Gonzalez, M. I., Baker, B. I., and Wilson, C. A. (1997) Stimulatory effect of melanin-concentrating hormone on luteinizing hormone release. Neuroendocrinology. 66(4):254-262.

Gonzalez, M. I., Kalia, V., Hole, D. R. and Wilson, C. A. (1997) α-melanocyte-stimulating hormone (α-MSH) and melanin-concentrating hormone (MCH) modify monoaminergic levels in the preoptic area of the rat. Peptides 18:387-392.

Gonzalez, M. I., Vazira, S., and Wilson, C. A. (1996) Behavioral effects of a-melanocyte-stimulating hormone (α-MSH) and melanin-concentrating hormone (MCH) after central administration in female rats. Peptides 17:171-177.

Graziano, M. P.; Hey, P. J.; Strader, C. D. (1996)The amino terminal domain of the glucagon-like peptide-1 receptor is a critical determinant of subtype specificity. Receptors Channels 4(1):9-17.

Grillon, S., Herve, C., Griffond, B., and Fellmann, D. (1997) Exploring the expression of the melanin-concentrating hormone messenger RNA in the rat lateral hypothalamus after goldthioglucose injection. Neuropeptides 31(2):131-136.

Guan, X. M.; Amend, A.; Strader, C. D. (1995) Determination of structural domains for G protein coupling and ligand binding in beta 3-adrenergic receptor. Mol. Pharmacol. 48(3):492-498 (September 1995).

Gundersen, C. B., Miledi, R., and Parker, I. (1983) Serotonin receptors induced by exogenous messenger RNA in Xenopus oocytes. Proc R Soc Lond B Biol Sci (Aug. 22, 1983) 219:1214 103-9.

Guy, A. P. and Gardner, C. R. (1985) Pharmacological characterisation of a modified social interaction model of anxiety. Neuropsychobiology, 13: 194-200.

Habert-Ortoli, E., et al.,(1994) Molecular cloning of a functional human galanin receptor. Proc Natl Acad Sci USA 91:9780-9783.

Herve, C. and Fellmann, D. (1997) Changes in rat melanin-concentrating hormone and dynorphin messenger ribonucleic acids induced by food deprivation. Neuropeptides 31(3):237-242.

Herrick-Davis, K., et al., (2000) Inverse agonist activity of atypical antipsychotic drugs at human 5-Hydroxytryptamine2C receptors. J. Pharmacol. Exp. Ther., 295(1): 226-32.

Hervieu, G. and Nahon, J. L. (1995) Pro-melanin concentrating hormone messenger ribonucleic acid and peptides expression in peripheral tissues of the rat. Neuroendocrinology. 61(4):348-364.

Hervieu, G., Segretain, D. and Nahon, J-L. (1996) Development and stage-dependent expression of melanin-concentrating hormone in mammalian germ cells. Biology of Reproduction 54:1161-1172.

Hervieu, G. J., et.al. (2000) The distribution of the mRNA and protein products of the melanin-concentrating hormone (MCH) receptor gene, slc-1, in the central nervous system of the rat. Eur. J. Neurosci. 12: 1194-1216.

Inui, A. (1999) Neuropeptide Y feeding receptors: are multiple subtypes involved? Trends Pharmacol Sci. 20:43-6.

Iversen, L. (2000) Neurotransmetter transporters: fruitful targets for CNS drug discovery. Mol. Psychiatry, 5(4): 357-62.

Javitch, J. A., et al, (1984) ³H-Mazindol binding associated with neuronal dopamine and norepinephrine uptake sites. Molecular Pharmacology 26: 35-44.

Jezova, D., et. al. (1992) Rat melanin-concentrating hormone stimulates adrenocorticotropin secretion: evidence for a site of action in brain regions protected by the blood brain barrier. Endocrinology 130:1021-1029.

Julius, D., et al.,(1988) Molecular characterization of a functional cDNA encoding the serotonin 1c receptor. Science 241:558-564.

Kalra, S. P., et al., (1999) Interacting appetite-regulating pathways in the hypothalamic regulation of body weight. Endocr Rev. 20:68-100.

Kauwachi, H., Kawazoe, I., Tsubokawa, M., Kishida, M. and Baker, B. I. (1983) Characterization of melanin-concentrating hormone in chum salmon pituitaries. Nature 305:321-333.

Kenakin, T. (1996) The classification of seven transmembrane receptors in recombinant expression systems. Pharmacol. Rev., 48(3): 413-63.

Kennett, G. A., et al., (1997) Anxiolytic-like actions of the selective 5-HT4 receptor antagonist SB-20470-A and SB-20766-A in rats. Neuropharmacology, 36(4-5): 707-712.

Kirby, L. G. and Lucki, I. (1997) Interaction between the forced swimming test and fluoxetine treatment on extracellular 5-hydroxytryptamine and 5-hydroxyindoleacetic acid in the rat. Stress, 2(4): 251-63.

Knigge, K. M., Baxter-Grillo, D., Speciale, J. and Wagner, J. (1996) Melanotropic peptides in the mammalian brain: The melanin-concentrating hormone. Peptides 17:1063-1073.

Knigge, K. M. and Wagner, J. E. (1997) Melanin-concentrating hormone (MCH) involvement in pentylenetetrazole (PTZ)-induced seizure in rat and guinea pig. Peptides 18(7):1095-1097.

Krapivinsky, G., Gordon, E. A., Wickman B., Velimirovic, B., Krapivinsky, L., Clapham, D. E. (1995) The G-protein-gated atrial K+channel IKACh is a heteromultimer of two inwardly rectifying K(+)-channel proteins. Nature 374:135-141.

Krapivinsky, G., Krapivinsky, L., Velimirovic, B., Wickman, K., Navarro, B., Clapham, D. E., (1995b) The cardiac inward rectifier K+ channel subunit, CIR, does not comprise the ATP-sensitive K+ channel, IKATP. J. Biol. Chem. 270: 28777-28779.

Kubo, Y., Reuveny, E., Slesinger, P. A., Jan, Y. N., Jan, L. Y. (1993) Primary structure and functional expression of a rat G-protein-coupled muscarinic potassium channel. Nature 364: 802-806.

Larhammar, D., et al., (1992) Cloning and functional expression of a human neuropeptide Y/peptide YY receptor of the Y1 type. J Biol Chem. 267:10935-10938.

Lazareno S. and Birdsall N. J.(1993) Estimation of competitive antagonist affinity from functional inhibition curvesusing the Gaddum, Schild and Cheng-Prusoff equations. Br J Pharmacol. 109:1110-1119.

Lazareno, S. and Birdsall N. J. M. (1993) Pharmacological characterization of acetylcholine stimulated [³⁵S]-GTPγS binding mediated by human muscarinic m1-m4 receptors: antagonist studies. Br. J. Pharmacology, 109: 1120-1127.

Lightowler, S., et al., (1994) Anxiolytic-like effect of paroxetine in a rat social interaction test. Pharmacol. Behav. Biochem., 49: 281-285.

Lucki, I. (1997) The forced swimming test as a model for core and component behavioral effects of antidepressant drugs. Behav. Pharmacol., 8: 523-528.

Ludwig, D. S., Mountjoy, K. G., Tatro, J. B., Gillette, J. A., Frederich, R. C., Flier, J. S., and Maratos-Flier, E. (1998) Melanin-concentrating hormone: a functional melanocortin antagonist in the hypothalamus. Am.J.Physiol.Endocrinol.Metab. 274(4):E627-E633.

Lutz, M. and Kenakin, T. (1999) Quantitative Molecular Pharmacology and Informatics in Drug Discovery, John Wiley & Sons, LTD, West Sussix, England. P. 153.

MacKenzie, F. J., Hunter, A. J., Daly, C., Wilson, C. A. (1984) Evidence that the dopaminergic incerto-hypothalamic tract has a stimulatory effect on ovulation and gonadotropin release. Neuroendocrinology 39:289-295.

Martin, R., et al. J. Tetrahedron Letters (1997), 38, 1633.

Masu, Y. et al. (1994) Nature 329:21583-21586.

McBride, R. B., Beckwith, B. E., Swenson, R. R., Sawyer, T. K., Hadley, M. E., Matsunaga, T. O. and Hruby, V. J. (1994) The actions of melanin-concentrating hormone (MCH) on passive avoidance in rats: A preliminary study. Peptides 15:757-759.

Melki, J., Abdelhak, S., Sheth, P., Bachelot, M. F., Burlet, P., Marcadet, A., Aicardi, J., Barois, A., Carriere, J. P., Fardeau, M., Fontan, D., Ponsot, G., Billette, T., Angelini, C., Barbosa, C., Ferriere, G., Lanzi, G., Ottolini, A., Babron, M. C., Cohen, D., Hanauer, A., Clerget-Darpoux, G., Lathrop, M., Munnich, A. and Frezal, J. (1990) Gene for chronic proximal spinal muscular atrophies maps to chromosome Sq. Nature (London) 344:767-768.

Miller, C. L., Hruby, V., Matsubaga, T., Bickford, P. (1993) α-MSH and MCH are functional antagonists in a CNS auditory paradigm. Peptides 14:1-10.

Miller, J., Germain, R. N., Efficient cell surface expression of class II MHC molecules in the absence of associated invariant chain. J.Exp.Med. 164:1478-1489 (1986).

Monsma, F. J. Jr., et al., (1993) Cloning and expression of a novel serotonin receptor with high affinity for tricyclic psychotropic drugs. Mol. Pharmacol., 43: 320-27.

Morishita, F., Hashito, K., Fujimoto, M. and Yamada, K. (1993) Possible involvement of pertussis toxin-sensitive GTP-binding protein in the a2-adrenoceptor-mediated melanosome-aggregation response of goldfish melanophores. J. Exp. Zoology 266:173-180.

Nahon, J. L., Presse, F., Bittencourt, J. C., Sawchenko, P., and Vale, W. (1989) The rat melanin-concentrating hormone mRNA encodes multiple putative neuropeptides coexpressed in the dorsolateral hypothalamus. Endocrinology 125:2056-2065.

Nahon, J-L. (1994) The melanin-concentrating hormone: from the peptide to the gene. Critical Rev. in Neurobiol 221:221-262.

Nonogaki, K., et al.,(1998) Leptin-independent hyperphagia and type 2 diabetes in mice with a mutated serotonin 5-HT2C receptor gene. Nature Medicine 4:1152-1156.

Otsuka, S. and Kobayashi, Y. (1964) A radioisotopic assay for monoamine oxidase determinations in human plasma. Biochem. Pharmacol., 13: 995-1006.

Owens, M. J. (1997) Neurotransmitter receptor and transporter binding profile of antidepressants and their metabolites. J. Pharm. Exp. Ther., 283: 1305-1322.

Page, M. E., et al., (1999) Serotonergic mediation of the effects of fluoxetine, but not desipramine, in the rat forced swim test. Psychopharmacology, 147: 162-167.

Parkes, D. G. (1996) Diuretic and natriuretic actions of melanin concentrating hormone in conscious sheep. J. Neuroendocrinol. 8:57-63.

Parkes, D. and Vale, W. (1993) Secretion of melanin-concentrating hormone and neuropeptide-EI from cultured rat hypothalamic cells. Endocrinology 131:1826-1831.

Pedeutour, F., Szpirer, C. and Nahon, J. L. (1994) Assignment of the human pro-melanin-concentrating hormone gene (PMCH) to chromosome 12q23-24 and two variant genes (PMCHL1 and PMCHL2) to chromosome 5p14 and 5q12-ql3. Genomics 19:31-37.

Porsolt, R. D. (1981) Behavioral despair. In Enna, SJ (ed) Antidepressants: neurochemical, behavioral and clinical perspectives. Raven Press, New York, pp. 121-139.

Porsolt, R. D., et al., (1978) Behavioral despair in rats: a new model sensitive to antidepressant treatments. Eur. J. Pharmacol., 47: 379-391.

Porsolt, R. D., et al., (1977) Depression: a new animal model sensitive to antidepressant treatments. Nature, 266: 730-732.

Presse, F., Hervieu, G., Imaki, T., Sawchenko, P. E., Vale, W., and Nahon, J-L. (1992) Rat melanin-concentrating hormone messenger ribonucleic acid expression: marked changes during development and after stress and glucocorticoid stimuli. Endocrinology 131:1241-1250.

Qu, D., Ludwig, D. S., Gammeltoft, S., Piper, M., Pelleymounter, M. A., Cullen, M. J., Foulds Mathes, W., Przypek, J., Kanarek, R. and Maratos-Flier, E. (1996) A role for melanin-concentrating hormone in the central regulation of feeding behaviour. Nature 380:243-247.

Qu, D., Mastaitis, J. W., Tritos, N. A. and Maratos-Flier, E. (1998) 80^{th} Annual Meeting of the Endocrine Society in New Orleans. Abs. # P1-494.

Quick, M. W., Lester, H. A. Methods for expression of excitability proteins in Xenopus oocytes. Meth. Neurosci. 19:261-279 (1994).

Reneric, J. P. and Lucki, I. (1998) Antidepressant behavioral effects by dual inhibition of monoamine reuptake in the rat forced swim test. Psychopharmacology, 136: 190-197.

Riva, M. A. and Creese, I. (1989) Comparison of two putatively selective radioligands for labeling central nervous system beta-adrenergic receptors: inadequacy of [3H]dihydroalprenolol. Mol. Pharmacol. 36: 201-210.

Rodgers, R. J., et al., (1997) Animal models of anxiety: an ethological perspective. Braz. J. Med. Biol. Res., 30: 289-304.

Rossi, M., Choi, S. J., O'Shea, D., Miyoshi, T., Ghatei, A. and Bloom, S. R. (1997) Melanin-concentrating hormone acutely stimulates feeding, but chronic administration has no effect on body weight. Endocrinology 138:351-355.

Sahu, A. (1998) Evidence suggesting that galanin (GAL), melanin-concentrating hormone (MCH), neurotensin (NT), proopiomelanocortin (POMC) and neuropeptide Y (NPY) are targets of leptin signaling in the hypothalamus. Endocrinology 139(2):795-798.

Saito, Y., et. al. (1999) Molecular characterization of the melanin-concentrating-hormone receptor. Nature 400:265-269.

Sakurai, T., Amemiya, A., Ishii, M., Matsuzaki, I., Chemelli, R. M. et al., (1998) Orexins and orexin receptors: A family of hypothalamic neuropeptides and G protein-coupled receptors that regulate feeding behavior. Cell 92:573-585.

Salon, J. A. and Owicki, J. C. (1995) Real-time measurements of receptor activity: Applications of microphysiometric techniques to receptor biology. In: Methods in Neuroscience 25:201-223 (Academic Press, 1995).

Sambrook, J., Fritsch, E. F., and Maniatis, T., In: Molecular Cloning: A Laboratory Manual, 2nd Edition (Cold Spring Harbor Laboratory, Cold Spring Harbor, N.Y.), 1989.

Sanchez, M., Baker, B. I. and Celis, M. (1997) Melanin-concentrating hormone (MCH) antagonizes the effects of α-MSH and neuropeptide E-I on grooming and locomotor activities in the rat. Peptides 18:393-396.

Schilling K., Luk, D., Morgan J., and Curran, T (1991) Regulation of a fos-lacZ fusion gene: A paradigm for quantitative analysis of stimulus transcription coupling. Proc. Nat. Acad. Sci (USA) 88:5665-5669.

Sherrington, R., Brynjolfsson, J., Petursson, H., Potter, M., Dudleston, K., Barraclough, B., Wasmuth, J., Dobbs, M. and Gurling, H. (1988) Localization of a susceptibility locus for schizophrenia on chromosome 5. Nature (London) 336:164-167.

Shimada, M., et al., (1998) Mice lacking melanin-concentrating hormone are hypophagic and lean. Nature 396:670-674.

Skofitsch, G., et. al. (1985) Immunohistochemical localization of a melanin concentrating hormone-like peptide in the rat brain. Brain Res. Bull. 15:635-639.

Smith. K. E., et al.,(1998) Cloned human and rat galanin GALR3 receptors. Pharmacology and activation of G-protein inwardly rectifying K+ channels. J Biol Chem 273:23321-23326.

Smith, K. E., et al.(1997) Expression cloning of a rat hypothalamic galanin receptor coupled to phosphoinositide turnover. J Biol Chem 272:24612-24616.

Spurney, R. F.; Coffman, T. M. (1997) The C-terminus of the thromboxane receptor contributes to coupling and desensitization in a mouse mesangial cell line. J. Pharmacol. Exp. Ther. 283(1):207-215 (Oct. 1997).

Srebnik, M., et al. J. Org. Chem. (1988), 53, 2916-2920.

Stuart, R. O., Sun, A., Bush, K. T., and Nigam, S. K. (1996) Dependence of epithelial intercellular junction biogenesis on thapsigargin-sensitive intracellular calcium stores. J Biol Chem (Jun. 7, 1996) 271(23):13636-41.

Svenssson, S. P., Norberg, T., Andersson, R. G., Grundstrom, N. and Karlsson, J. O. G. (1991) MCH-induced pigment aggregation in teleost melanophores is associated with a cAMP reduction. Life Sci. 48:2043-2046.

Takahashi, T., Neher, E., and Sakmann, B. (1987) Rat brain serotonin receptors in Xenopus oocytes are coupled by intracellular calcium to endogenous channels. Proc Natl Acad Sci USA (1987 July) 84(14):5063-7.

Tatsumi, M., et al., (1997) Pharmacological profile of antidepressants and related compounds at human monoamine transporters. Eur. J. Pharmacol., 340(2-3): 249-58.

Tian, W., Duzic, E., Lanier, S., and Deth R. (1994) Determinants of α-Adrenergic Receptor Activation of G protein: Evidence for a Precoupled Receptor/G protein State. Molecular Pharmacology, 45:524-531.

Toumaniantz, G., Bittencourt, J. C., and Nahon, J. L. (1996) The rat melanin-concentrating hormone gene encodes an additional putative protein in a different reading frame. Endocrinology 137:4518-4521.

Treit, D. (1985) Animal models for the study of anti-anxiety agents: a review. Neurosci. Biobehav. Rev., 9: 203-222.

Tritos, N. A., et al., (2000) The obese phenotype of melanin concentrating hormone overexpressing mice. Abstract #1192, The Endocrine Society 82nd Annual Meeting, June 21-24.

Twells, R., Weber, J., Orozco, G., Farrell, M., Williamson, R. and Chamberlain, S. (1992) Chromosomal assignment of the locus causing olivo-ponto-cerebellar atrophy (SCA2) in a cuban founder population. Cytogent. Cell. Cenet. 61:262-265.

Underwood, D. J., Strader, C. D., Rivero, R., Patchett, A. A., Greenlee ,W., and Prendergast, K. (1994) Structural model of antagonist and agonist binding to the angiotensin II, AT1 subtype, G protein coupled receptor. Chem Biol (1994 December) 1(4):211-21.

Viale, A., Zhixing, Y., Breton, C., Pedeutour, F., Coquerel, A., Jordan, D., Nahon, J. L. (1997) The melanin-concentrating hormone gene in human: flanking region analysis, fine chromosome mapping, and tissue-specific expression. Mol. Brain Res. 46:243-255.

Westbrook, C. A., Neuman, W. L., McPherson, J., Camper, S., Wasmuth, J., Plaetke, R. and Williamson, R. (1992) Report of the second international workshop on human chromosome 5 mapping. Cytogenet. Cell. Genet. 61:225-231.

Yalkinoglu, A. O., Spreyer, P., Bechem, M., Apeler, N., and Wohlfeil, S. (1995) Induction of c-fos expression in rat vascular smooth muscle reporter cell by selective activation of the thrombin receptor. J. Receptor and Signal Transduction, 15(1-4):117-130.

Zamir, N., et. al. (1986) Distribution of immunoreactive melatonin-concentrating hormone in the central nervous system of the rat. Brain Res. 373 (1-2):240-245.

What is claimed is:

1. An isolated nucleic acid encoding a human MCH1 receptor or a mutant of such human MCH1 receptor which is activated by MCH or an analog or homolog thereof.

2. The nucleic acid of claim 1, wherein the nucleic acid is DNA.

3. The DNA of claim 2, wherein the DNA is cDNA.

4. The DNA of claim 2, wherein the DNA is genomic DNA.

5. The nucleic acid of claim 1, wherein the nucleic acid is RNA.

6. The nucleic acid of claim 1, wherein the human MCH1 receptor has an amino acid sequence identical to that encoded by the plasmid pEXJ.HR-TL231 (ATCC Accession No. 203197).

7. The nucleic acid of claim 1, wherein the human MCH1 receptor comprises an amino acid sequence as shown in FIG. 2 (SEQ ID NO: 2).

8. The nucleic acid of claim 1, wherein the mutant human MCH1 receptor comprises an amino acid sequence as shown in FIG. 13 (SEQ ID NO: 26).

9. The nucleic acid of claim 1, wherein the mutant human MCH1 receptor comprises an amino acid sequence as shown in FIG. 14 (SEQ ID NO: 27).

10. The nucleic acid of claim 1, wherein the mutant human MCH1 receptor comprises an amino acid sequence as shown in FIG. 15 (SEQ ID NO: 28).

11. A purified human MCH1 receptor protein.

12. A vector comprising the nucleic acid of claim 1.

13. The vector of claim 12 adapted for expression in a cell which comprises the regulatory elements necessary for expression of the nucleic acid in the cell operatively linked to the nucleic acid encoding the receptor so as to permit expression thereof, wherein the cell is a bacterial, amphibian, yeast, insect or mammalian cell.

14. The vector of claim 13, wherein the vector is a baculovirus.

15. The vector of claim 12, wherein the vector is a plasmid.

16. The plasmid of claim 15 designated pEXJ.HR-TL231 (ATCC Accession No. 203197).

17. A cell comprising the vector of claim 13.

18. A cell of claim 17, wherein the cell is a non-mammalian cell.

19. A cell of claim 18, wherein the non-mammalian cell is a Xenopus oocyte cell or a Xenopus melanophore cell.

20. A cell of claim 17, wherein the cell is a mammalian cell.

21. A mammalian cell of claim 20, wherein the cell is a COS-7 cell, a 293 human embryonic kidney cell, a NIH-3T3 cell, a LM(tk−) cell, a mouse Y1 cell, or a CHO cell.

22. An insect cell comprising the vector of claim 13.

23. An insect cell of claim 22, wherein the insect cell is an Sf9 cell, an Sf21 cell or a Trichoplusia ni 5B-4 cell.

24. A membrane preparation isolated from the cell of claim 17.

25. A nucleic acid probe comprising at least 15 nucleotides which specifically hybridizes with a nucleic acid encoding a human MCH1 receptor, wherein the probe has a unique sequence corresponding to a sequence present within one of the two strands of the nucleic acid encoding a human MCH1 receptor present in plasmid pEXJ.HR-T231 (ATCC Accession No. 203197).

26. A nucleic acid probe comprising at least 15 nucleotides which specifically hybridizes with a nucleic acid encoding a human MCH1 receptor, wherein the probe has a unique sequence corresponding to a sequence present within (a) the nucleic acid sequence shown in FIG. 1 (SEQ ID NO: 1) or (b) the reverse complement thereof.

27. The nucleic acid probe of claim 25 or 26, wherein the nucleic acid is DNA.

28. The nucleic acid probe of claim 25 or 26, wherein the nucleic acid is RNA.

29. An antisense oligonucleotide having a sequence capable of specifically hybridizing to the RNA of claim 5, so as to prevent translation of the RNA.

30. An antisense oligonucleotide having a sequence capable of specifically hybridizing to the genomic DNA of claim 4.

31. An antisense oligonucleotide of claim 29 or 30, wherein the oligonucleotide comprises chemically modified nucleotides or nucleotide analogues.

32. An antibody capable of binding to a human MCH1 receptor encoded by the nucleic acid of claim 1.

33. An agent capable of competitively inhibiting the binding of the antibody of claim 32 to a human MCH1 receptor.

34. An antibody of claim 32, wherein the antibody is a monoclonal antibody or antisera.

35. A pharmaceutical composition comprising (a) an amount of the oligonucleotide of claim 29 capable of passing through a cell membrane and effective to reduce expression of a human MCH1 receptor and (b) a pharmaceutically acceptable carrier capable of passing through the cell membrane.

36. A pharmaceutical composition of claim 35, wherein the oligonucleotide is coupled to a substance which inactivates mRNA.

37. A pharmaceutical composition of claim 36, wherein the substance which inactivates mRNA is a ribozyme.

38. A pharmaceutical composition of claim 35, wherein the pharmaceutically acceptable carrier comprises a structure which binds to a human MCH1 receptor on a cell capable of being taken up by the cells after binding to the structure.

39. A pharmaceutical composition of claim 35, wherein the pharmaceutically acceptable carrier is capable of binding to a human MCH1 receptor which is specific for a selected cell type.

40. A pharmaceutical composition which comprises an amount of the antibody of claim 32 effective to block binding of a ligand to a human MCH1 receptor and a pharmaceutically acceptable carrier.

41. A transgenic, nonhuman mammal expressing DNA encoding a human MCH1 receptor of claim 1.

42. A transgenic, nonhuman mammal comprising a homologous recombination knockout of the native human MCH1 receptor.

43. A transgenic, nonhuman mammal whose genome comprises antisense DNA complementary to the DNA encoding a human MCH1 receptor of claim 1 so placed within the genome as to be transcribed into antisense mRNA which is complementary to mRNA encoding the human MCH1 receptor and which hybridizes to mRNA encoding the human MCH1 receptor, thereby reducing its translation.

44. The transgenic, nonhuman mammal of claim 41 or 42, wherein the DNA encoding the human MCH1 receptor additionally comprises an inducible promoter.

45. The transgenic, nonhuman mammal of claim 41 or 42, wherein the DNA encoding the human MCH1 receptor additionally comprises tissue specific regulatory elements.

46. A transgenic, nonhuman mammal of claim 41, 42, or 43, wherein the transgenic, nonhuman mammal is a mouse.

47. A process for identifying a chemical compound which specifically binds to a mammalian MCH1 receptor which comprises contacting cells comprising DNA encoding, and expressing on their cell surface, the mammalian MCH1 receptor, with the compound under conditions suitable for binding, and detecting specific binding of the chemical compound to the mammalian MCH1 receptor, wherein the cells do not normally express the mammalian MCH1 receptor and the DNA encoding the mammalian MCH1 receptor (a) hybridizes to a nucleic acid having the defined sequence shown in FIG. 1 (SEQ ID NO: 1) under low stringency conditions or a sequence complementary thereto and (b) is further characterized by its ability to cause a change in the pH of a culture of CHO cells when a MCH1 ligand is added to the culture and the CHO cells contain the nucleic acid which hybridized to the nucleic acid having the defined sequence or its complement.

48. A process for identifying a chemical compound which specifically binds to a mammalian MCH1 receptor which comprises contacting a membrane preparation from cells comprising DNA encoding, and expressing on their cell surface, the mammalian MCH1 receptor, with the compound under conditions suitable for binding, and detecting specific binding of the chemical compound to the mammalian MCH1 receptor, wherein the cells do not normally express the mammalian MCH1 receptor and the DNA encoding the mammalian MCH1 receptor (a) hybridizes to a nucleic acid having the defined sequence shown in FIG. 1 (SEQ ID NO: 1) under low stringency conditions or a sequence complementary thereto and (b) is further characterized by its ability to cause a change in the pH of a culture of CHO cells when a MCH1 ligand is added to the culture and the CHO cells contain the nucleic acid which hybridized to the nucleic acid having the defined sequence or its complement.

49. The process of claim 47 or 48, wherein the mammalian MCH1 receptor is a human MCH1 receptor.

50. The process of claim 47 or 48, wherein the mammalian MCH1 receptor is a rat MCH1 receptor.

51. The process of claim 47 or 48, wherein the mammalian MCH1 receptor has substantially the same amino acid sequence as the sequence of the human MCH1 receptor encoded by plasmid pEXJ.HR-TL231 (ATCC Accession No. 203197).

52. The process of claim 47 or 48, wherein the mammalian MCH1 receptor comprises substantially the same amino acid sequence as that shown in FIG. 2 (SEQ ID NO: 2).

53. The process of claim 47 or 48, wherein the mammalian MCH1 receptor comprises the amino acid sequence shown in FIG. 2 (SEQ ID NO: 2).

54. The process of claim 47 or 48, wherein the mammalian MCH1 receptor comprises the amino acid sequence shown in FIG. 13 (SEQ ID NO: 26).

55. The process of claim 47 or 48, wherein the mammalian MCH1 receptor comprises the amino acid sequence shown in FIG. 14 (SEQ ID NO: 27).

56. The process of claim 47 or 48, wherein the mammalian MCH1 receptor comprises the amino acid sequence shown in FIG. 15 (SEQ ID NO: 28).

57. The process of claim 47 or 48, wherein the compound is not previously known to bind to a mammalian MCH1 receptor.

58. A compound identified by the process of claim 57.

59. A process of claim 47 or 48, wherein the cell is an insect cell.

60. The process of claim 47 or 48, wherein the cell is a mammalian cell.

61. The process of claim 60, wherein the cell is nonneuronal in origin.

62. The process of claim 61, wherein the nonneuronal cell is a COS-7 cell, 293 human embryonic kidney cell, a CHO cell, a NIH-3T3 cell, a mouse Y1 cell, or a LM(tk−cell.

63. A process of claim 60, wherein the compound is a compound not previously known to bind to a mammalian MCH1 receptor.

64. A compound identified by the process of claim 63.

65. A process involving competitive binding for identifying a chemical compound which specifically binds to a mammalian MCH1 receptor which comprises contacting cells expressing on their cell surface the mammalian MCH1 receptor, with both the chemical compound and a second chemical compound known to bind to the receptor, and separately with only the second chemical compound, under conditions suitable for binding of both compounds, and detecting specific binding of the chemical compound to the mammalian MCH1 receptor, a decrease in the binding of the second chemical compound to the mammalian MCH1 receptor in the presence of the chemical compound indicating that the chemical compound binds to the mammalian MCH1 receptor, wherein the cells do not normally express the mammalian MCH1 receptor and the DNA encoding the mammalian MCH1 receptor (a) hybridizes to a nucleic acid having the defined sequence shown in FIG. 1 (SEQ ID NO: 1) under low stringency conditions or a sequence complementary thereto and (b) is further characterized by its ability to cause a change in the pH of a culture of CHO cells when a MCH1 ligand is added to the culture and the CHO cells contain the nucleic acid which hybridized to the nucleic acid having the defined sequence or its complement.

66. A process involving competitive binding for identifying a chemical compound which specifically binds to a mammalian MCH1 receptor which comprises contacting a membrane preparation from cells expressing on their cell surface the mammalian MCH1 receptor, with both the chemical compound and a second chemical compound known to bind to the receptor, and separately with only the second chemical compound, under conditions suitable for binding of both compounds, and detecting specific binding of the chemical compound to the mammalian MCH1 receptor, a decrease in the binding of the second chemical compound to the mammalian MCH1 receptor in the presence of the chemical compound indicating that the chemical compound binds to the mammalian MCH1 receptor, wherein the cells do not normally express the mammalian MCH1 receptor and the DNA encoding the mammalian MCH1 receptor (a) hybridizes to a nucleic acid having the defined sequence shown in FIG. 1 (SEQ ID NO: 1) under low stringency conditions or a sequence complementary thereto and (b) is further characterized by its ability to cause a change in the pH of a culture of CHO cells when a MCH1 ligand is added to the culture and the CHO cells contain the nucleic acid which hybridized to the nucleic acid having the defined sequence or its complement.

67. A process of claim 65 or 66, wherein the mammalian MCH1 receptor is a human MCH1 receptor or a mutant of such human MCH1 receptor which is activated by MCH or an analog or homolog thereof.

68. A process of claim 65 or 66, wherein the mammalian MCH1 receptor is a rat MCH1 receptor.

69. The process of claim 65 or 66, wherein the cell is an insect cell.

70. The process of claim 65 or 66, wherein the cell is a mammalian cell.

71. The process of claim 70, wherein the cell is nonneuronal in origin.

72. The process of claim 71, wherein the nonneuronal cell is a COS-7 cell, 293 human embryonic kidney cell, a CHO cell, a NIH-3T3 cell, a mouse Y1 cell, or a LM(tk−cell.

73. The process of claim 70, wherein the compound is not previously known to bind to a mammalian MCH1 receptor.

74. A compound identified by the process of claim 73.

75. A method of screening a plurality of chemical compounds not known to bind to a mammalian MCH1 receptor to identify a compound which specifically binds to the mammalian MCH1 receptor, which comprises
(a) contacting cells transfected with and expressing DNA encoding the mammalian MCH1 receptor with the plurality of compounds not known to bind specifically to the mammalian MCH1 receptor, under conditions permitting binding of compounds known to bind the mammalian MCH1 receptor;
(b) determining whether the binding of a compound known to bind to the mammalian MCH1 receptor is reduced in the presence of the compounds within the plurality of compounds, relative to the binding of the compound in the absence of the plurality of compounds; and if so
(c) separately determining the binding to the mammalian MCH1 receptor of compounds included in the plurality of compounds, so as to thereby identify the compound which specifically binds to the mammalian MCH1 receptor.

76. A method of screening a plurality of chemical compounds not known to bind to a mammalian MCH1 receptor to identify a compound which specifically binds to the mammalian MCH1 receptor, which comprises
(a) contacting a membrane preparation from cells transfected with and expressing DNA encoding the mammalian MCH1 receptor with the plurality of compounds not known to bind specifically to the mammalian MCH1 receptor under conditions permitting binding of compounds known to bind the mammalian MCH1 receptor;
(b) determining whether the binding of a compound known to bind to the mammalian MCH1 receptor is reduced in the presence of the compounds within the plurality of compounds, relative to the binding of the compound in the absence of the plurality of compounds; and if so
(c) separately determining the binding to the mammalian MCH1 receptor of compounds included in the plurality of compounds, so as to thereby identify the compound which specifically binds to the mammalian MCH1 receptor.

77. A method of claim 75 or 76, wherein the mammalian MCH1 receptor is a human MCH1 receptor or a mutant of such human MCH1 receptor which is activated by MCH or an analog or homolog thereof.

78. A method of claim 75 or 76, wherein the mammalian MCH1 receptor is a rat MCH1 receptor.

79. A method of claim 75 or 76, wherein the cell is a mammalian cell.

80. A method of claim 79, wherein the mammalian cell is non-neuronal in origin.

81. The method of claim 80, wherein the non-neuronal cell is a COS-7 cell, a 293 human embryonic kidney cell, a LM(tk−) cell, a CHO cell, a mouse Y1 cell, or an NIH-3T3 cell.

82. A method of detecting expression of a mammalian MCH1 receptor by detecting the presence of mRNA coding for the mammalian MCH1 receptor which comprises obtaining total mRNA from the cell and contacting the mRNA so obtained with the nucleic acid probe of any of claims 25, 26, 27, or 28 under hybridizing conditions, detecting the presence of mRNA hybridizing to the probe, and thereby detecting the expression of the mammalian MCH1 receptor by the cell.

83. A method of detecting the presence of a mammalian MCH1 receptor on the surface of a cell which comprises contacting the cell with the antibody of claim 32 under conditions permitting binding of the antibody to the receptor, detecting the presence of the antibody bound to the cell, and thereby detecting the presence of the mammalian MCH1 receptor on the surface of the cell.

84. A method of determining the physiological effects of varying levels of activity of human MCH1 receptors which comprises producing a transgenic, nonhuman mammal of claim 44 whose levels of human MCH1 receptor activity are varied by use of an inducible promoter which regulates human MCH1 receptor expression.

85. A method of determining the physiological effects of varying levels of activity of human MCH1 receptors which comprises producing a panel of transgenic, nonhuman mammals of claim 44, each expressing a different amount of human MCH1 receptor.

86. A method for identifying an antagonist capable of alleviating an abnormality, wherein the abnormality is alleviated by decreasing the activity of a human MCH1 receptor comprising administering a compound to the transgenic, nonhuman mammal of claim 41, 44, 45, or 46, and determining whether the compound alleviates the physical and behavioral abnormalities displayed by the transgenic, nonhuman mammal as a result of overactivity of a human MCH1 receptor, the alleviation of the abnormality identifying the compound as an antagonist.

87. An antagonist identified by the method of claim 86.

88. A pharmaceutical composition comprising an antagonist of claim 87 and a pharmaceutically acceptable carrier.

89. A method of treating an abnormality in a subject wherein the abnormality is alleviated by decreasing the activity of a human MCH1 receptor which comprises administering to the subject an effective amount of the pharmaceutical composition of claim 88, thereby treating the abnormality.

90. A method for identifying an agonist capable of alleviating an abnormality in a subject wherein the abnormality is alleviated by increasing the activity of a human MCH1 receptor comprising administering a compound to the transgenic, nonhuman mammal of claim 41, 44, 45, or 46, and determining whether the compound alleviates the physical and behavioral abnormalities displayed by the transgenic, nonhuman mammal, the alleviation of the abnormality identifying the compound as an agonist.

91. An agonist identified by the method of claim 90.

92. A pharmaceutical composition comprising an agonist of claim 91 and a pharmaceutically acceptable carrier.

93. A method of treating an abnormality in a subject wherein the abnormality is alleviated by increasing the activity of a human MCH1 receptor which comprises administering to the subject an effective amount of the pharmaceutical composition of claim 92, thereby treating the abnormality.

94. A method for diagnosing a predisposition to a disorder associated with the activity of a specific mammalian allele which comprises:
(a) obtaining DNA of subjects suffering from the disorder;
(b) performing a restriction digest of the DNA with a panel of restriction enzymes;
(c) electrophoretically separating the resulting DNA fragments on a sizing gel;
(d) contacting the resulting gel with a nucleic acid probe capable of specifically hybridizing with a unique sequence included within the sequence of a nucleic acid molecule encoding a human MCH1 receptor and labeled with a detectable marker;
(e) detecting labeled bands which have hybridized to the DNA encoding a human MCH1 receptor of claim 1 labeled with a detectable marker to create a unique band pattern specific to the DNA of subjects suffering from the disorder;
(f) preparing DNA obtained for diagnosis by steps (a)-(e); and
(g) comparing the unique band pattern specific to the DNA of subjects suffering from the disorder from step (e) and the DNA obtained for diagnosis from step (f) to determine whether the patterns are the same or different and to diagnose thereby predisposition to the disorder if the patterns are the same.

95. The method of claim 94, wherein a disorder associated with the activity of a specific mammalian allele is diagnosed.

96. A method of preparing the purified human MCH1 receptor of claim 11 which comprises:
(a) inducing cells to express the human MCH1 receptor;
(b) recovering the human MCH1 receptor from the induced cells; and
(c) purifying the human MCH1 receptor so recovered.

97. A method of preparing the purified human MCH1 receptor of claim 11 which comprises:
(a) inserting nucleic acid encoding the human MCH1 receptor in a suitable vector;
(b) introducing the resulting vector in a suitable host cell;
(c) placing the resulting cell in suitable condition permitting the production of the isolated human MCH1 receptor;
(d) recovering the human MCH1 receptor produced by the resulting cell; and
(e) purifying the human MCH1 receptor so recovered.

98. A process for determining whether a chemical compound is a mammalian MCH1 receptor agonist which comprises contacting cells transfected with and expressing DNA encoding the mammalian MCH1 receptor with the compound under conditions permitting the activation of the mammalian MCH1 receptor, and detecting an increase in mammalian MCH1 receptor activity, so as to thereby determine whether the compound is a mammalian MCH1 receptor agonist.

99. A process for determining whether a chemical compound is a mammalian MCH1 receptor antagonist which comprises contacting cells transfected with and expressing DNA encoding the mammalian MCH1 receptor with the compound in the presence of a known mammalian MCH1 receptor agonist, under conditions permitting the activation of the mammalian MCH1 receptor, and detecting a decrease in mammalian MCH1 receptor activity, so as to thereby determine whether the compound is a mammalian MCH1 receptor antagonist.

100. A process of claim 98 or 99, wherein the mammalian MCH1 receptor is a human MCH1 receptor or a mutant of such human MCH1 receptor which is activated by MCH or an analog or homolog thereof.

101. A process of claim 98 or 99, wherein the mammalian MCH1 receptor is a rat MCH1 receptor.

102. A pharmaceutical composition which comprises an amount of a mammalian MCH1 receptor agonist determined by the process of claim 98 effective to increase activity of a mammalian MCH1 receptor and a pharmaceutically acceptable carrier.

103. A pharmaceutical composition of claim 102, wherein the mammalian MCH1 receptor agonist is not previously known.

104. A pharmaceutical composition which comprises an amount of a mammalian MCH1 receptor antagonist determined by the process of claim 99 effective to reduce activity of a mammalian MCH1 receptor and a pharmaceutically acceptable carrier.

105. A pharmaceutical composition of claim 104, wherein the mammalian MCH1 receptor antagonist is not previously known.

106. A process for determining whether a chemical compound specifically binds to and activates a mammalian MCH1 receptor, which comprises contacting cells producing a second messenger response and expressing on their cell surface the mammalian MCH1 receptor, wherein such cells do not normally express the mammalian MCH1 receptor, with the chemical compound under conditions suitable for activation of the mammalian MCH1 receptor, and measuring the second messenger response in the presence and in the absence of the chemical compound, a change in the second messenger response in the presence of the chemical compound indicating that the compound activates the mammalian MCH1 receptor.

107. The process of claim 106, wherein the second messenger response comprises chloride channel activation and the change in second messenger is an increase in the level of inward chloride current.

108. A process for determining whether a chemical compound specifically binds to and inhibits activation of a mammalian MCH1 receptor, which comprises separately contacting cells producing a second messenger response and expressing on their cell surface the mammalian MCH1 receptor, wherein such cells do not normally express the mammalian MCH1 receptor, with both the chemical compound and a second chemical compound known to activate the mammalian MCH1 receptor, and with only the second chemical compound, under conditions suitable for activation of the mammalian MCH1 receptor, and measuring the second messenger response in the presence of only the second chemical compound and in the presence of both the second chemical compound and the chemical compound, a smaller change in the second messenger response in the presence of both the chemical compound and the second chemical compound than in the presence of only the second chemical compound indicating that the chemical compound inhibits activation of the mammalian MCH1 receptor.

109. The process of claim 108, wherein the second messenger response comprises chloride channel activation and the change in second messenger response is a smaller increase in the level of inward chloride current in the presence of both the chemical compound and the second chemical compound than in the presence of only the second chemical compound.

110. A process of any of claims 106, 107, 108, or 109, wherein the mammalian MCH1 receptor is a human MCH1 receptor or a mutant of such human MCH1 receptor which is activated by MCH or an analog or homolog thereof.

111. A process of any of claims 106, 107, 108, or 109, wherein the mammalian MCH1 receptor is a rat MCH1 receptor.

112. The process of any of claims 106, 107, 108, 109, or 110, wherein the cell is an insect cell.

113. The process of any of claims 106, 107, 108, 109, or 110, wherein the cell is a mammalian cell.

114. The process of claim 113, wherein the mammalian cell is nonneuronal in origin.

115. The process of claim 114, wherein the nonneuronal cell is a COS-7 cell, CHO cell, 293 human embryonic kidney cell, NIH-3T3 cell or LM(tk−) cell.

116. The process of claim 106, 107, 108, or 109, wherein the compound is not previously known to bind to a mammalian MCH1 receptor.

117. A compound determined by the process of claim 116.

118. A pharmaceutical composition which comprises an amount of a mammalian MCH1 receptor agonist determined by the process of claim 106 or 107 effective to increase activity of a mammalian MCH1 receptor and a pharmaceutically acceptable carrier.

119. A pharmaceutical composition of claim 118, wherein the mammalian MCH1 receptor agonist is not previously known.

120. A pharmaceutical composition which comprises an amount of a mammalian MCH1 receptor antagonist determined by the process of claim 108 or 109 effective to reduce activity of a mammalian MCH1 receptor and a pharmaceutically acceptable carrier.

121. A pharmaceutical composition of claim 120, wherein the mammalian MCH1 receptor antagonist is not previously known.

122. A method of screening a plurality of chemical compounds not known to activate a mammalian MCH1 receptor to identify a compound which activates the mammalian MCH1 receptor which comprises:
(a) contacting cells transfected with and expressing the mammalian MCH1 receptor with the plurality of compounds not known to activate the mammalian MCH1 receptor, under conditions permitting activation of the mammalian MCH1 receptor;
(b) determining whether the activity of the mammalian MCH1 receptor is increased in the presence of the compounds; and if so
(c) separately determining whether the activation of the mammalian MCH1 receptor is increased by each compound included in the plurality of compounds, so as to thereby identify the compound which activates the mammalian MCH1 receptor.

123. A method of claim 122, wherein the mammalian MCH1 receptor is a human MCH1 receptor or a mutant of such human MCH1 receptor which is activated by MCH or an analog or homolog thereof.

124. A method of claim 122, wherein the mammalian MCH1 receptor is a rat MCH1 receptor.

125. A method of screening a plurality of chemical compounds not known to inhibit the activation of a mammalian MCH1 receptor to identify a compound which inhibits the activation of the mammalian MCH1 receptor, which comprises:
(a) contacting cells transfected with and expressing the mammalian MCH1 receptor with the plurality of compounds in the presence of a known mammalian MCH1 receptor agonist, under conditions permitting activation of the mammalian MCH1 receptor;
(b) determining whether the activation of the mammalian MCH1 receptor is reduced in the presence of the plurality of compounds, relative to the activation of the mammalian MCH1 receptor in the absence of the plurality of compounds; and if so
(c) separately determining the inhibition of activation of the mammalian MCH1 receptor for each compound included in the plurality of compounds, so as to thereby identify the compound which inhibits the activation of the mammalian MCH1 receptor.

126. A method of claim 125, wherein the mammalian MCH1 receptor is a human MCH1 receptor or a mutant of such human MCH1 receptor which is activated by MCH or an analog or homolog thereof.

127. A method of claim 125, wherein the mammalian MCH1 receptor is a rat MCH1 receptor.

128. A method of any of claims 123, 124, 125, 126, or 127, wherein the cell is a mammalian cell.

129. A method of claim 128, wherein the mammalian cell is non-neuronal in origin.

130. The method of claim 129, wherein the non-neuronal cell is a COS-7 cell, a 293 human embryonic kidney cell, a LM(tk−) cell or an NIH-3T3 cell.

131. A pharmaceutical composition comprising a compound identified by the method of claim 123 or 124 effective to increase mammalian MCH1 receptor activity and a pharmaceutically acceptable carrier.

132. A pharmaceutical composition comprising a compound identified by the method of claim 125 or 126 effective to decrease mammalian MCH1 receptor activity and a pharmaceutically acceptable carrier.

133. A method of treating an abnormality in a subject wherein the abnormality is alleviated by increasing the activity of a mammalian MCH1 receptor which comprises administering to the subject an amount of a compound which is a mammalian MCH1 receptor agonist effective to treat the abnormality.

134. A method of claim 133, wherein the abnormality is a regulation of a steroid or pituitary hormone disorder, an epinephrine release disorder, a gastrointestinal disorder, a cardiovascular disorder, an electrolyte balance disorder, hypertension, diabetes, a respiratory disorder, asthma, a reproductive function disorder, an immune disorder, an endocrine disorder, a musculoskeletal disorder, a neuroendocrine disorder, a cognitive disorder, a memory disorder, a sensory modulation and transmission disorder, a motor coordination disorder, a sensory integration disorder, a motor integration disorder, a dopaminergic function disorder, a sensory transmission disorder, an olfaction disorder, a sympathetic innervation disorder, pain, psychotic behavior, morphine tolerance, opiate addiction, an affective disorder, a stress-related disorder, a fluid-balance disorder, a seizure disorder, or migraine.

135. A method of treating an abnormality in a subject wherein the abnormality is alleviated by decreasing the activity of a mammalian MCH1 receptor which comprises administering to the subject an amount of a compound which is a mammalian MCH1 receptor antagonist effective to treat the abnormality.

136. A method of claim 135, wherein the abnormality is a regulation of a steroid or pituitary hormone disorder, an epinephrine release disorder, a gastrointestinal disorder, a cardiovascular disorder, an electrolyte balance disorder, hypertension, diabetes, a respiratory disorder, asthma, a reproductive function disorder, an immune disorder, an endocrine disorder, a musculoskeletal disorder, a neuroendocrine disorder, a cognitive disorder, a memory disorder, a sensory modulation and transmission disorder, a motor coordination disorder, a sensory integration disorder, a motor integration disorder, a dopaminergic function disorder, a sensory transmission disorder, an olfaction disorder, a sympathetic innervation disorder, pain, psychotic behavior, morphine tolerance, opiate addiction, an affective disorder, a stress-related disorder, a fluid-balance disorder, a seizure disorder, or migraine.

137. A process for making a composition of matter which specifically binds to a mammalian MCH1 receptor which comprises identifying a chemical compound using the process of any of claims 47, 48, 65, 66, 75, or 76 and then synthesizing the chemical compound or a novel structural and functional analog or homolog thereof.

138. A process for making a composition of matter which specifically binds to a mammalian MCH1 receptor which comprises identifying a chemical compound using the process of any of claims 98, 106, or 122 and then synthesizing the chemical compound or a novel structural and functional analog or homolog thereof.

139. A process for making a composition of matter which specifically binds to a mammalian MCH1 receptor which comprises identifying a chemical compound using the process of any of claims 99, 108, or 125 and then synthesizing the chemical compound or a novel structural and functional analog or homolog thereof.

140. The process of any of claims 137, 138, or 139, wherein the mammalian MCH1 receptor is a human MCH1 receptor or a mutant of such human MCH1 receptor which is activated by MCH or an analog or homolog thereof.

141. The process of any of claims 137, 138, or 139, wherein the mammalian MCH1 receptor is a human MCH1 receptor.

142. A process for preparing a composition which comprises admixing a pharmaceutically acceptable carrier and a therapeutically effective amount of a chemical compound identified by the process of any of claims 47, 48, 65, 66, 75, or 76 or a novel structural and functional analog or homolog thereof.

143. A process for preparing a composition which comprises admixing a pharmaceutically acceptable carrier and a therapeutically effective amount of a chemical compound identified by the process of any of claims 98, 106, or 122 or a novel structural and functional analog or homolog thereof.

144. A process for preparing a composition which comprises admixing a pharmaceutically acceptable carrier and a therapeutically effective amount of a chemical compound identified by the process of any of claims 99, 108, or 125 or a novel structural and functional analog or homolog thereof.

145. The process of any of claims 142, 143, or 144, wherein the mammalian MCH1 receptor is a human MCH1 receptor or a mutant of such human MCH1 receptor which is activated by MCH or an analog or homolog thereof.

146. The process of any of claims 142, 143, or 144, wherein the mammalian MCH1 receptor is a rat MCH1 receptor.

147. A process for determining whether a chemical compound is a human MCH1 receptor antagonist which comprises contacting cells transfected with and expressing DNA encoding the human MCH1 receptor with the compound in the presence of a known human MCH1 receptor agonist, under conditions permitting the activation of the human MCH1 receptor, and detecting a decrease in human MCH1 receptor activity, so as to thereby determine whether the compound is a human MCH1 receptor antagonist, wherein the DNA encoding the human MCH1 receptor comprises the sequence shown in FIG. 1 (Seq. ID No. 1) or contained in plasmid pEXJ.HR-TL231 (ATCC Accession No. 203197), the known human MCH1 receptor agonist is MCH or a homolog or analog of MCH, and the cells do not express the MCH1 receptor prior to transfecting them.

148. A process for determining whether a chemical compound specifically binds to and inhibits activation of a human MCH1 receptor, which comprises separately contacting cells expressing on their cell surface the human MCH1 receptor and producing a second messenger response upon activation of the human MCH1 receptor, wherein such cells do not normally express the human MCH1 receptor and the DNA encoding the human MCH1 receptor comprises the sequence shown in FIG. 1 (Seq. ID No. 1) or contained in plasmid PEXJ.HR-TL231 (ATCC Accession No. 203197), with both the chemical compound and a second chemical compound known to activate the human MCH1 receptor, and with only the second chemical compound, under conditions suitable for activation of the human MCH1 receptor, and measuring the second messenger response in the presence of only the second chemical compound and in the presence of both the second chemical compound and the chemical compound, a smaller change in the second messenger response in the presence of both the chemical compound and the second chemical compound than in the presence of only the second chemical compound indicating that the chemical compound inhibits activation of the human MCH1 receptor, wherein the second chemical compound is MCH or a homolog or analog of MCH.

149. The process of claim 148, wherein the second messenger response comprises chloride channel activation and the change in second messenger response is a smaller increase in the level of inward chloride current in the presence of both the chemical compound and the second chemical compound than in the presence of only the second chemical compound.

150. A method of screening a plurality of chemical compounds not known to inhibit the activation of a human MCH1 receptor to identify a compound which inhibits the activation of the human MCH1 receptor, which comprises:
(a) contacting cells transfected with and expressing the human MCH1 receptor, wherein such cells do not normally express the human MCH1 receptor and the DNA encoding the human MCH1 receptor comprises the sequence shown in FIG. 1 (Seq. ID No. 1) or contained in plasmid pEXJ.HR-TL231 (ATCC Accession No. 203197), with the plurality of compounds in the presence of a known human MCH1 receptor agonist, under conditions permitting activation of the human MCH1 receptor, wherein the known MCH1 receptor agonist is MCH or a homolog or analog of MCH;
(b) determining whether the activation of the human MCH1 receptor is reduced in the presence of the plurality of compounds, relative to the activation of the human MCH1 receptor in the absence of the plurality of compounds; and if so
(c) separately determining the extent of inhibition of activation of the human MCH1 receptor for each compound included in the plurality of compounds, so as to thereby identify the compound which inhibits the activation of the human MCH1 receptor.

151. The process of any of claims 147, 148 or 150, wherein the cell is an insect cell.

152. The process of any of claims 147, 148 or 150, wherein the cell is a mammalian cell.

153. The process of any of claims 147, 148 or 150, wherein the cell is a mammalian cell which is nonneuronal in origin.

154. The process of any of claims 147, 148 or 150, wherein the cell is a COS-7 cell, a CHO cell, a 293 human embryonic kidney cell, a NIH-3T3 cell, a mouse Y1 cell, or a LM(tk−) cell.

155. A process for making a composition of matter which specifically binds to a human MCH1 receptor which comprises identifying a chemical compound which specifically binds to the human MCH1 receptor and then synthesizing the chemical compound or a structural and functional analog or homolog thereof, wherein the chemical compound is identified as binding to the human MCH1 receptor by a process involving competitive binding which comprises contacting cells expressing on their cell surface the human MCH1 receptor, with both the chemical compound and a second chemical compound known to bind to the receptor, and separately with only the second chemical compound, under conditions suitable for binding of both compounds, and detecting the extent of specific binding of the chemical compound to the human MCH1 receptor, a decrease in the binding of the second chemical compound to the human MCH1 receptor in the presence of the chemical compound indicating that the chemical compound binds to the human MCH1 receptor, wherein the cells do not normally express the human MCH1 receptor, the human MCH1 receptor is encoded by nucleic acid comprising the sequence shown in FIG. 1 (Seq. ID No. 1) or contained in plasmid pEXJ.HR-TL231 (ATCC Accession No. 203197), and the second chemical compound is MCH or a homolog or analog of MCH.

156. A process for making a composition of matter which specifically binds to a human MCH1 receptor which comprises identifying a chemical compound which specifically binds to the human MCH1 receptor and then synthesizing the chemical compound or a structural and functional analog or homolog thereof, wherein the chemical compound is identified as binding to the human MCH1 receptor by a process involving competitive binding which comprises contacting a membrane preparation from cells expressing on their cell surface the human MCH1 receptor, with both the chemical compound and a second chemical compound known to bind to the receptor, and separately with only the second chemical compound, under conditions suitable for binding of both compounds, and detecting the extent of specific binding of the chemical compound to the human MCH1 receptor, a decrease in the binding of the second chemical compound to the human MCH1 receptor in the presence of the chemical compound indicating that the chemical compound binds to the human MCH1 receptor, wherein the cells do not normally express the human MCH1 receptor, the human MCH1 receptor is encoded by nucleic acid comprising the sequence shown in FIG. 1 (Seq. ID No. 1) or contained in plasmid pEXJ.HR-TL231 (ATCC Accession No. 203197), and the second chemical compound is MCH or a homolog or analog of MCH.

157. A process for making a composition of matter which is a human MCH1 receptor antagonist which comprises identifying a chemical compound which is a human MCH1 receptor antagonist and then synthesizing the chemical compound or a structural and functional analog or homolog thereof, wherein the chemical compound is identified as a human MCH1 receptor antagonist by a process which comprises contacting cells transfected with and expressing DNA encoding the human MCH1 receptor with the compound in the presence of a known human MCH1 receptor agonist, under conditions permitting the activation of the human MCH1 receptor, and detecting a decrease in human MCH1 receptor activity, so as to thereby determine whether the compound is a human MCH1 receptor antagonist, wherein the cells do not normally express the human MCH1 receptor, the human MCH1 receptor is encoded by nucleic acid comprising the sequence shown in FIG. 1 (Seq. ID No. 1) or contained in plasmid pEXJ.HR-TL231 (ATCC Accession No. 203197), and the known human MCH1 receptor agonist is MCH or a homolog or analog of MCH.

158. A process for making a composition of matter which specifically binds to and inhibits the activation of a human MCH1 receptor which comprises identifying a chemical compound which specifically binds to and inhibits the activation of the human MCH1 receptor and then synthesizing the chemical compound or a structural and functional analog or homolog thereof, wherein the chemical compound is identified as binding to and inhibiting the activation of the human MCH1 receptor by a process which comprises separately contacting cells expressing on their cell surface the human MCH1 receptor and producing a second messenger response upon activation of the human MCH1 receptor, wherein such cells do not normally express the human MCH1 receptor and the human MCH1 receptor is encoded by nucleic acid comprising the sequence shown in FIG. 1 (Seq. ID No. 1) or contained in plasmid pEXJ.HR-TL231 (ATCC Accession No. 203197), with both the chemical compound and a second chemical compound known to activate the human MCH1 receptor, and with only the second chemical compound, under conditions suitable for activation of the human MCH1 receptor, and measuring the second messenger response in the presence of only the second chemical compound and in the presence of both the second chemical compound and the chemical compound, a smaller change in the second messenger response in the presence of both the chemical compound and the second chemical compound than in the presence of only the second chemical compound indicating that the chemical compound inhibits activation of the human MCH1 receptor, wherein the second chemical compound is MCH or a homolog or analog of MCH.

159. The process of claim 158, wherein the second messenger response comprises chloride channel activation and the change in second messenger response is a smaller increase in the level of inward chloride current in the presence of both the chemical compound and the second chemical compound than in the presence of only the second chemical compound.

160. A process for preparing a composition which comprises identifying a chemical compound which specifically binds to a human MCH1 receptor, and then admixing a carrier and the chemical compound or a structural and functional analog or homolog thereof, wherein the chemical compound is identified as binding to the human MCH1 receptor by a process involving competitive binding which comprises contacting cells expressing on their cell surface the human MCH1 receptor, with both the chemical compound and a second chemical compound known to bind to the receptor, and separately with only the second chemical compound, under conditions suitable for binding of both compounds, and detecting the extent of specific binding of the chemical compound to the human MCH1 receptor, a decrease in the binding of the second chemical compound to the human MCH1 receptor in the presence of the chemical compound indicating that the chemical compound binds to the human MCH1 receptor, wherein the cells do not normally express the human MCH1 receptor, the human MCH1 receptor is encoded by nucleic acid comprising the sequence shown in FIG. 1 (Seq. ID No. 1) or contained in plasmid pEXJ.HR-TL231 (ATCC Accession No. 203197), and the second chemical compound is MCH or a homolog or analog of MCH.

161. A process for preparing a composition which comprises identifying a chemical compound which specifically binds to a human MCH1 receptor, and then admixing a carrier and the chemical compound or a structural and functional analog or homolog thereof, wherein the chemical compound is identified as binding to the human MCH1 receptor by a process involving competitive binding which comprises contacting a membrane preparation from cells expressing on their cell surface the human MCH1 receptor, with both the chemical compound and a second chemical compound known to bind to the receptor, and separately with only the second chemical compound, under conditions suitable for binding of both compounds, and detecting the extent of specific binding of the chemical compound to the human MCH1 receptor, a decrease in the binding of the second chemical compound to the human MCH1 receptor in the presence of the chemical compound indicating that the chemical compound binds to the human MCH1 receptor, wherein the cells do not normally express the human MCH1 receptor, the human MCH1 receptor is encoded by nucleic acid comprising the sequence shown in FIG. 1 (Seq. ID No. 1) or contained in plasmid pEXJ.HR-TL231 (ATCC Accession No. 203197), and the second chemical compound is MCH or a homolog or analog of MCH.

162. A process for preparing a composition which comprises identifying a chemical compound which is a human MCH1 receptor antagonist, and then admixing a carrier and the chemical compound or a structural and functional analog or homolog thereof, wherein the chemical compound is identified as a human MCH1 receptor antagonist by a process which comprises contacting cells transfected with and expressing DNA encoding the human MCH1 receptor with the compound in the presence of a known human MCH1 receptor agonist, under conditions permitting the activation of the human MCH1 receptor, and detecting a decrease in human MCH1 receptor activity, so as to thereby determine whether the compound is a human MCH1 receptor antagonist, wherein the cells do not normally express the human MCH1 receptor, the human MCH1 receptor is encoded by nucleic acid comprising the sequence shown in FIG. 1 (Seq. ID No. 1) or contained in plasmid pEXJ.HR-TL231 (ATCC Accession No. 203197), and the known human MCH1 receptor agonist is MCH or a homolog or analog of MCH.

163. A process for preparing a composition which comprises identifying a chemical compound which specifically binds to and inhibits the activation of a human MCH1 receptor, and then admixing a carrier and the chemical compound or a structural and functional analog or homolog thereof, wherein the chemical compound is identified as binding to and inhibiting activation of the human MCH1 receptor by a process which comprises separately contacting cells expressing on their cell surface the human MCH1 receptor and producing a second messenger response upon activation of the human MCH1 receptor, wherein such cells do not normally express the human MCH1 receptor and the human MCH1 receptor is encoded by nucleic acid comprising the sequence shown in FIG. 1 (Seq. ID No. 1) or contained in plasmid pEXJ.HR-TL231 (ATCC Accession No. 203197), with both the chemical compound and a second chemical compound known to activate the human MCH1 receptor, and with only the second chemical compound, under conditions suitable for activation of the human MCH1 receptor, and measuring the second messenger response in the presence of only the second chemical compound and in the presence of both the second chemical compound and the chemical compound, a smaller change in the second messenger response in the presence of both the chemical compound and the second chemical compound than in the presence of only the second chemical compound indicating that the chemical compound inhibits activation of the human MCH1 receptor, wherein the second chemical compound is MCH or a homolog or analog of MCH.

164. The process of claim 163, wherein the second messenger response comprises chloride channel activation and the change in second messenger response is a smaller increase in the level of inward chloride current in the presence of both the chemical compound and the second chemical compound than in the presence of only the second chemical compound.

165. The process of any of claims 155, 156, 157, 158, 160, 161, 162, or 163, wherein the cell is an insect cell.

166. The process of any of claims 155, 156, 157, 158, 160, 161, 162, or 163, wherein the cell is a mammalian cell.

167. The process of claim 166, wherein the mammalian cell is nonneuronal in origin.

168. The process of claim 167, wherein the nonneuronal cell is a COS-7 cell, a 293 human embryonic kidney cell, a CHO cell, a NIH-3T3 cell, a mouse Y1 cell, or a LM(tk−) cell.

169. A method of treating an eating disorder or obesity in a subject which comprises administering to the subject a therapeutically effective amount of an MCH1 antagonist which inhibits the activation of the MCH1 receptor.

170. A method of claim 169, wherein the MCH1 antagonist additionally inhibits the activation of the MCH1 receptor with an antagonist potency which is at least 30-fold greater than the antagonist potency with which the MCH1 antagonist inhibits the activation of each of the 5-HT2C and MC-4 receptors.

171. A method of claim 170, wherein the MCH1 antagonist additionally inhibits the activation of the MCH1 receptor with an antagonist potency which is at least 10-fold greater than the antagonist potency with which the MCH1 antagonist inhibits the activation of each of the NPY1, NPY5, GALR1, GALR2, and GALR3 receptors.

172. A method of claim 170, wherein the MCH1 antagonist additionally inhibits the activation of the MCH1 receptor with an antagonist potency which is at least 100-fold greater than the antagonist potency with which the MCH1 antagonist inhibits the activation of each of the 5-HT2C and MC-4 receptors.

173. A method of claim 172, wherein the MCH1 antagonist additionally inhibits the activation of the MCH1 receptor with an antagonist potency which is at least 100-fold greater than the antagonist potency with which the MCH1 antagonist inhibits the activation of each of the NPY1, NPY5, GALR1, GALR2, and GALR3 receptors.

174. A method of claim 169, wherein the MCH1 antagonist additionally inhibits the activation of the MCH1 receptor with an antagonist potency which is at least 30-fold greater than the binding affinity with which the MCH1 antagonist binds to each of the 5-HT2C and MC-4 receptors.

175. A method of claim 174, wherein the MCH1 antagonist additionally inhibits the activation of the MCH1 receptor with an antagonist potency which is at least 10-fold greater than the binding affinity with which the MCH1 antagonist binds to each of the NPY1, NPY5, GALR1, GALR2, and GALR3 receptors.

176. A method of claim 174, wherein the MCH1 antagonist additionally inhibits the activation of the MCH1 receptor with an antagonist potency which is at least 100-fold greater than the binding affinity with which the MCH1 antagonist binds to each of the 5-HT2C and MC-4 receptors.

177. A method of claim 176, wherein the MCH1 antagonist additionally inhibits the activation of the MCH1 receptor with an antagonist potency which is at least 100-fold greater than the binding affinity with which the MCH1 antagonist binds to each of the NPY1, NPY5, GALR1, GALR2, and GALR3 receptors.

178. A method of claim 169, wherein the MCH1 antagonist additionally binds to the MCH1 receptor with a binding affinity which is at least 30-fold greater than the binding affinity with which the MCH1 antagonist binds to each of the 5-HT2C and MC-4 receptors.

179. A method of claim 178, wherein the MCH1 antagonist additionally binds to the MCH1 receptor with a binding affinity which is at least 10-fold greater than the binding affinity with which the MCH1 antagonist binds to each of the NPY1, NPY5, GALR1, GALR2, and GALR3 receptors.

180. A method of claim 178, wherein the MCH1 antagonist additionally binds to the MCH1 receptor with a binding affinity which is at least 100-fold greater than the binding affinity with which the MCH1 antagonist binds to each of the 5-HT2C and MC-4 receptors.

181. A method of claim 180, wherein the MCH1 antagonist additionally binds to the MCH1 receptor with a binding affinity which is at least 100-fold greater than the binding affinity with which the MCH1 antagonist binds to each of the NPY1, NPY5, GALR1, GALR2, and GALR3 receptors.

182. A method of claim 169, wherein the MCH1 antagonist additionally binds to the MCH1 receptor with a binding affinity which is at least 30-fold greater than the binding affinity with which the MCH1 antagonist binds to the dopamine D2 receptor.

183. A method of claim 169, wherein the MCH1 antagonist additionally binds to the MCH1 receptor with a binding affinity which is at least 30-fold greater than the binding affinity with which the MCH1 antagonist binds to the histamine H1 receptor.

184. A method of claim 169, wherein the MCH1 antagonist additionally binds to the MCH1 receptor with a binding affinity which is at least 100-fold greater than the binding affinity with which the MCH1 antagonist binds to the dopamine D2 receptor.

185. A method of claim 169, wherein the MCH1 antagonist additionally binds to the MCH1 receptor with a binding affinity which is at least 100-fold greater than the binding affinity with which the MCH1 antagonist binds to the histamine H1 receptor.

186. A method of claim 169, wherein the MCH1 antagonist additionally binds to the MCH1 receptor with a binding affinity which is at least 200-fold greater than the binding affinity with which the MCH1 antagonist binds to the dopamine D2 receptor.

187. A method of claim 169, wherein the MCH1 antagonist additionally binds to the MCH1 receptor with a binding affinity which is at least 200-fold greater than the binding affinity with which the MCH1 antagonist binds to the histamine Hi receptor.

188. A method of claim 169, wherein the MCH1 antagonist additionally binds to the MCH1 receptor with a binding affinity which is at least 10-fold greater than the binding affinity with which the MCH1 antagonist binds to the α_{1A} adrenoceptor.

189. A method of claim 169, wherein the MCH1 antagonist additionally binds to the MCH1 receptor with a binding affinity which is at least 100-fold greater than the binding affinity with which the MCH1 antagonist binds to the α_{1A} adrenoceptor.

190. A method of claim 169, wherein the MCH1 antagonist additionally binds to the α_{1A} adrenoceptor with a binding affinity which is no more than 10-fold greater than the binding affinity with which the MCH1 antagonist binds to the MCH1 receptor.

191. A method of claim 169, wherein the MCH1 antagonist additionally binds to the α_{1A} adrenoceptor with a binding affinity which is no more than 100-fold greater than the binding affinity with which the MCH1 antagonist binds to the MCH1 receptor.

192. A method of treating an eating disorder in a subject which comprises administering to the subject a therapeutically effective amount of an MCH1 agonist which activates the MCH1 receptor.

193. A method of claim 192, wherein the MCH1 agonist additionally activates the MCH1 receptor with an agonist potency which is at least 30-fold greater than the agonist potency with which the MCH1 agonist activates each of the 5-HT2C and MC-4 receptors.

194. A method of claim 193, wherein the MCH1 agonist additionally activates the MCH1 receptor with an agonist potency which is at least 10-fold greater than the agonist potency with which the MCH1 agonist activates each of the NPY1, NPY5, GALR1, GALR2, and GALR3 receptors.

195. A method of claim 193, wherein the MCH1 agonist additionally activates the MCH1 receptor with an agonist potency which is at least 100-fold greater than the agonist potency with which the MCH1 agonist activates each of the 5-HT2C and MC-4 receptors.

196. A method of claim 195, wherein the MCH1 agonist additionally activates the MCH1 receptor with an agonist potency which is at least 100-fold greater than the agonist potency with which the MCH1 agonist activates each of the NPY1, NPY5, GALR1, GALR2, and GALR3 receptors.

197. A method of any one of claims 192, 193, 194, 195, or 196, wherein the eating disorder is anorexia nervosa.

198. A method of treating depression and/or anxiety in a subject which comprises administering to the subject a composition comprising a pharmaceutically acceptable carrier and a therapeutically effective amount of a MCH1 antagonist, wherein:
(a) (1) the MCH1 antagonist does not inhibit the activity of central monoamine oxidase A greater than 50 percent, at a concentration of 10 mM; and (2) the MCH1 antagonist does not inhibit the activity of central monoamine oxidase B greater than 50 percent, at a concentration of 10 mM; and
(b) the MCH1 antagonist binds to the MCH1 receptor with a binding affinity at least ten-fold higher than the binding affinity with which it binds to each of the following transporters: serotonin transporter, norepinephrine transporter, and dopamine transporter.

199. The method of claim 198, wherein the MCH1 antagonist also binds to the MCH1 receptor with a binding affinity at least ten-fold higher than the binding affinity with which it binds to each of the human 5HT_{1A}, human 5HT_{1B}, human 5HT_{1D}, human 5HT_{1E}, human 5HT_{1F}, human 5HT_{2A}, rat 5HT_{2C}, human 5HT₄, human 5HT₆ and human 5HT₇ receptors.

200. The method of claim 198, wherein the MCH1 antagonist also binds to the MCH1 receptor with a binding affinity at least ten-fold higher than the binding affinity with which it binds to the human histamine H₁ and H₂ receptors.

201. The method of claim 198, wherein the MCH1 antagonist also binds to the MCH1 receptor with a binding affinity at least ten-fold higher than the binding affinity with which it binds to the human dopamine D₁, D₂, D₃, D₄ and D₅ receptors.

202. The method of claim 198, wherein the MCH1 antagonist also binds to the MCH1 receptor with a binding affinity at least ten-fold higher than the binding affinity with which it binds to the human α_{1A} adrenoceptor, the human α_{1B} adrenoceptor and the human α_{1D} adrenoceptor.

203. The method of claim 198, wherein the MCH1 antagonist also binds to the MCH1 receptor with a binding affinity at least ten-fold higher than the binding affinity with which it binds to the human α_{2A} adrenoceptor, the human α_{2B} adrenoceptor and the human α_{2C} adrenoceptor.

204. The method of claim 198, wherein the MCH1 antagonist does not inhibit the activity of central monoamine oxidase A greater than 60 percent.

205. The method of claim 198, wherein the MCH1 antagonist does not inhibit the activity of central monoamine oxidase B greater than 60 percent.

206. The method of claim 198, wherein the MCH1 antagonist does not inhibit the activity of central monoamine oxidase A greater than 70 percent.

207. The method of claim 198, wherein the MCH1 antagonist does not inhibit the activity of central monoamine oxidase B greater than 70 percent.


SEQUENCE LISTING (28 sequences)

SEQ ID NO: 1 - DNA, 1269 bp, Homo sapiens
atgtcagtgggagccatgaagaagggagtggggagggcagttgggcttggaggcggcagcggctgccaggctacggaggaagacccccttcccgactgcggggcttgcgctccgggacaaggtggcaggcgctggaggctgccgcagcctgcgtgggtggaggggagctcagctcggttgtgggagcaggcgaccggcactggctggatggacctggaagcctcgctgctgcccactggtcccaatgccagcaacacctctgatggccccgataacctcacttcagcaggatcacctcctcgcacggggagcatctcctacatcaacatcatcatgccttcggtgttcggcaccatctgcctcctgggcatcatcgggaactccacggtcatcttcgcggtcgtgaagaagtccaagctgcactggtgcaacaacgtccccgacatcttcatcatcaacctctcggtagtagatctcctctttctcctgggcatgcccttcatgatccaccagctcatgggcaatggggtgtggcactttggggagaccatgtgcaccctcatcacggccatggatgccaatagtcagttcaccagcacctacatcctgaccgccatggccattgaccgctacctggccactgtccaccccatctcttccacgaagttccggaagccctctgtggccaccctggtgatctgcctcctgtgggccctctccttcatcagcatcacccctgtgtggctgtatgccagactcatccccttcccaggaggtgcagtgggctgcggcatacgcctgcccaacccagacactgacctctactggttcaccctgtaccagtttttcctggcctttgccctgccttttgtggtcatcacagccgcatacgtgaggatcctgcagcgcatgacgtcctcagtggcccccgcctcccagcgcagcatccggctgcggacaaagagggtgacccgcacagccatcgccatctgtctggtcttctttgtgtgctgggcaccctactatgtgctacagctgacccagttgtccatcagccgcccgaccctcacctttgtctacttatacaatgcggccatcagcttgggctatgccaacagctgcctcaacccctttgtgtacatcgtgctctgtgagacgttccgcaaacgcttggtcctgtcggtgaagcctgcagcccaggggcagcttcgcgctgtcagcaacgctcagacggctgacgaggagaggacagaaagcaaaggcacctga

SEQ ID NO: 2 - protein, 422 aa, Homo sapiens
MSVGAMKKGVGRAVGLGGGSGCQATEEDPLPDCGACAPGQGGRRWRLPQPAWVEGSSARLWEQATGTGWMDLEASLLPTGPNASNTSDGPDNLTSAGSPPRTGSISYINIIMPSVFGTICLLGIIGNSTVIFAVVKKSKLHWCNNVPDIFIINLSVVDLLFLLGMPFMIHQLMGNGVWHFGETMCTLITAMDANSQFTSTYILTAMAIDRYLATVHPISSTKFRKPSVATLVICLLWALSFISITPVWLYARLIPFPGGAVGCGIRLPNPDTDLYWFTLYQFFLAFALPFVVITAAYVRILQRMTSSVAPASQRSIRLRTKRVTRTAIAICLVFFVCWAPYYVLQLTQLSISRPTLTFVYLYNAAISLGYANSCLNPFVYIVLCETFRKRLVLSVKPAAQGQLRAVSNAQTADEERTESKGT

SEQ ID NO: 3 - DNA, 1214 bp, Rattus norvegicus
gcaggcgacctgcaccggctgcatggatctgcaaacctcgttgctgtccactggccccaatgccagcaacatctccgatggccaggataatctcacattgccggggtcacctcctcgcacagggagtgtctcctacatcaacatcattatgccttccgtgtttggtaccatctgtctcctgggcatcgtgggaaactccacggtcatctttgctgtggtgaagaagtccaagctacactggtgcagcaacgtccccgacatcttcatcatcaacctctctgtggtggatctgctcttcctgctgggcatgcctttcatgatccaccagctcatggggaacggcgtctggcactttggggaaaccatgtgcaccctcatcacagccatggacgccaacagtcagttcactagcacctacatcctgactgccatgaccattgaccgctacttggccaccgtccaccccatctcctccaccaagttccggaagccctccatggccaccctggtgatctgcctcctgtgggcgctctccttcatcagtatcacccctgtgtggctctacgccaggctcattcccttcccagggggtgctgtgggctgtggcatccgcctgccaaacccggacactgacctctactggttcactctgtaccagtttttcctggcctttgcccttccgtttgtggtcattaccgccgcatacgtgaaaatactacagcgcatgacgtcttcggtggccccagcctcccaacgcagcatccggcttcggacaaagagggtgacccgcacggccattgccatctgtctggtcttctttgtgtgctgggcaccctactatgtgctgcagctgacccagctgtccatcagccgcccgaccctcacgtttgtctacttgtacaacgcggccatcagcttgggctatgctaacagctgcctgaacccctttgtgtacatagtgctctgtgagacctttcgaaaacgcttggtgttgtcagtgaagcctgcagcccaggggcagctccgcacggtcagcaacgctcagacagctgatgaggagaggacagaaagcaaaggcacctgacaattccccagtcgcctccaagtcaggccaccccatcaaaccgtggggagagatactgagattaaacccaaggctaccctgggagaatgcagaggctggaggctgggggcttgtagcaaccacattccac

SEQ ID NO: 4 - protein, 353 aa, Rattus norvegicus
MDLQTSLLSTGPNASNISDGQDNLTLPGSPPRTGSVSYINIIMPSVFGTICLLGIVGNSTVIFAVVKKSKLHWCSNVPDIFIINLSVVDLLFLLGMPFMIHQLMGNGVWHFGETMCTLITAMDANSQFTSTYILTAMTIDRYLATVHPISSTKFRKPSMATLVICLLWALSFISITPVWLYARLIPFPGGAVGCGIRLPNPDTDLYWFTLYQFFLAFALPFVVITAAYVKILQRMTSSVAPASQRSIRLRTKRVTRTAIAICLVFFVCWAPYYVLQLTQLSISRPTLTFVYLYNAAISLGYANSCLNPFVYIVLCETFRKRLVLSVKPAAQGQLRTVSNAQTADEERTESKGT

SEQ ID NO: 5 - DNA, 26 bp, Artificial Sequence
  Description of Artificial Sequence primer/ probe
gggaactccacggtcatcttcgcggt

SEQ ID NO: 6 - DNA, 26 bp, Artificial Sequence
  Description of Artificial Sequence primer/ probe
tagcggtcaatggccatggcggtcag

SEQ ID NO: 7 - DNA, 45 bp, Artificial Sequence
  Description of Artificial Sequence primer/ probe
ctcctgggcatgcccttcatgatccaccagctcatgggcaatggg

SEQ ID NO: 8 - DNA, 25 bp, Artificial Sequence
  Description of Artificial Sequence primer/ probe
cttctaggcctgtacggaagtgtta

SEQ ID NO: 9 - DNA, 27 bp, Artificial Sequence
  Description of Artificial Sequence primer/ probe
gttgtggtttgtccaaactcatcaatg

SEQ ID NO: 10 - DNA, 37 bp, Artificial Sequence
  Description of Artificial Sequence primer/ probe
cgcggatccattatgtctgcactccgaaggaaatttg

SEQ ID NO: 11 - DNA, 38 bp, Artificial Sequence
  Description of Artificial Sequence primer/ probe
cgcgaattcttatgtgaagcgatcagagttcatttttc

SEQ ID NO: 12 - DNA, 34 bp, Artificial Sequence
  Description of Artificial Sequence primer/ probe
gcgggatccgctatggctggtgattctaggaatg

SEQ ID NO: 13 - DNA, 29 bp, Artificial Sequence
  Description of Artificial Sequence primer/ probe
ccggaattcccctcacaccgagcccctgg

SEQ ID NO: 14 - DNA, 20 bp, Artificial Sequence
  Description of Artificial Sequence primer/ probe
tcagctcggttgtgggagca

SEQ ID NO: 15 - DNA, 18 bp, Artificial Sequence
  Description of Artificial Sequence primer/ probe
cttggacttcttcacgac

SEQ ID NO: 16 - protein, 100 aa, Artificial Sequence
  Description of Artificial Sequence mutated human MCH1
MSVGAMKKGVGTAVGLGGGSGCQATEEDPLPDCGACAPGQGGRRWRLPQPAWVEGSSARLWEQATGTGWADLEASLLPTGPNASNTSDGPDNLTSAGSPP

SEQ ID NO: 17 - protein, 100 aa, Artificial Sequence
  Description of Artificial Sequence mutated human MCH1
MSVGAAKKGVGRAVGLGGGSGCQATEEDPLPDCGACAPGQGGRRWRLPQPAWVEGSSARLWEQATGTGWADLEASLLPTGPNASNTSDGPDNLTSAGSPP

SEQ ID NO: 18 - DNA, 31 bp, Artificial Sequence
  Description of Artificial Sequence primer/ probe
cggcactggctgggcggacctggaagcctcg

SEQ ID NO: 19 - DNA, 31 bp, Artificial Sequence
  Description of Artificial Sequence primer/ probe
cgaggcttccaggtccgcccagccagtgccg

SEQ ID NO: 20 - DNA, 32 bp, Artificial Sequence
  Description of Artificial Sequence primer/ probe
atgtcagtgggagccgcgaagaagggagtggg

SEQ ID NO: 21 - DNA, 32 bp, Artificial Sequence
  Description of Artificial Sequence primer/ probe
cccactcccttcttcgcggctcccactgacat

SEQ ID NO: 22 - DNA, 33 bp, Artificial Sequence
  Description of Artificial Sequence primer/ probe
taatgtgtctaggtggcgtcagtgggagccatg

SEQ ID NO: 23 - DNA, 33 bp, Artificial Sequence
  Description of Artificial Sequence primer/ probe
catggctcccactgacgccacctagacacatta

SEQ ID NO: 24 - DNA, 37 bp, Artificial Sequence
  Description of Artificial Sequence primer/ probe
tgacactaagcttcactggctggatggacctggaagc

SEQ ID NO: 25 - DNA, 24 bp, Artificial Sequence
  Description of Artificial Sequence primer/ probe
gcccaggagaaagaggagatctac

SEQ ID NO: 26 - protein, 422 aa, Artificial Sequence
  Description of Artificial Sequence mutated human MCH1
MSVGAMKKGVGRAVGLGGGSGCQATEEDPLPDCGACAPGQGGRRWRLPQPAWVEGSSARLWEQATGTGWADLEASLLPTGPNASNTSDGPDNLTSAGSPPRTGSISYINIIMPSVFGTICLLGIIGNSTVIFAVVKKSKLHWCNNVPDIFIINLSVVDLLFLLGMPFMIHQLMGNGVWHFGETMCTLITAMDANSQFTSTYILTAMAIDRYLATVHPISSTKFRKPSVATLVICLLWALSFISITPVWLYARLIPFPGGAVGCGIRLPNPDTDLYWFTLYQFFLAFALPFVVITAAYVRILQRMTSSVAPASQRSIRLRTKRVTRTAIAICLVFFVCWAPYYVLQLTQLSISRPTLTFVYLYNAAISLGYANSCLNPFVYIVLCETFRKRLVLSVKPAAQGQLRAVSNAQTADEERTESKGT

SEQ ID NO: 27 - protein, 422 aa, Artificial Sequence
  Description of Artificial Sequence mutated human MCH1
MSVGAAKKGVGRAVGLGGGSGCQATEEDPLPDCGACAPGQGGRRWRLPQPAWVEGSSARLWEQATGTGWADLEASLLPTGPNASNTSDGPDNLTSAGSPPRTGSISYINIIMPSVFGTICLLGIIGNSTVIFAVVKKSKLHWCNNVPDIFIINLSVVDLLFLLGMPFMIHQLMGNGVWHFGETMCTLITAMDANSQFTSTYILTAMAIDRYLATVHPISSTKFRKPSVATLVICLLWALSFISITPVWLYARLIPFPGGAVGCGIRLPNPDTDLYWFTLYQFFLAFALPFVVITAAYVRILQRMTSSVAPASQRSIRLRTKRVTRTAIAICLVFFVCWAPYYVLQLTQLSISRPTLTFVYLYNAAISLGYANSCLNPFVYIVLCETFRKRLVLSVKPAAQGQLRAVSNAQTADEERTESKGT

SEQ ID NO: 28 - protein, 353 aa, Artificial Sequence
  Description of Artificial Sequence mutated human MCH1
MDLEASLLPTGPNASNTSDGPDNLTSAGSPPRTGSISYINIIMPSVFGTICLLGIIGNSTVIFAVVKKSKLHWCNNVPDIFIINLSVVDLLFLLGMPFMIHQLMGNGVWHFGETMCTLITAMDANSQFTSTYILTAMAIDRYLATVHPISSTKFRKPSVATLVICLLWALSFISITPVWLYARLIPFPGGAVGCGIRLPNPDTDLYWFTLYQFFLAFALPFVVITAAYVRILQRMTSSVAPASQRSIRLRTKRVTRTAIAICLVFFVCWAPYYVLQLTQLSISRPTLTFVYLYNAAISLGYANSCLNPFVYIVLCETFRKRLVLSVKPAAQGQLRAVSNAQTADEERTESKGT